(19) United States
(12) Patent Application Publication
(10) Pub. No.: US 2014/0193827 A1
(43) Pub. Date: Jul. 10, 2014

(54) CHARACTERIZING A GLATIRAMER ACETATE RELATED DRUG PRODUCT

(71) Applicants: Rivka Schwartz, Tel-Mond (IL); Shlomo Bakshi, Moshav Salit (IL); Kevin Daniel Fowler, Cambridge, MA (US); Fadi George Towfic, Somerville, MA (US); Jason Michael Funt, Malden, MA (US); Benjamin James Zeskind, Cambridge, MA (US); Maksym Artomov, Kirkwood, MO (US)

(72) Inventors: Rivka Schwartz, Tel-Mond (IL); Shlomo Bakshi, Moshav Salit (IL); Kevin Daniel Fowler, Cambridge, MA (US); Fadi George Towfic, Somerville, MA (US); Jason Michael Funt, Malden, MA (US); Benjamin James Zeskind, Cambridge, MA (US); Maksym Artomov, Kirkwood, MO (US)

(73) Assignee: TEVA PHARMACEUTICAL INDUSTRIES, LTD., Petach-Tikva (IL)

(21) Appl. No.: 14/147,167

(22) Filed: Jan. 3, 2014

Related U.S. Application Data
(60) Provisional application No. 61/819,481, filed on May 3, 2013.
(60) Provisional application No. 61/749,228, filed on Jan. 4, 2013.

(51) Int. Cl. G01N 33/50

(52) U.S. Cl. CPC G01N 33/5023
USPC 435/6.13

(57) ABSTRACT

The present invention provides a process for characterizing a glatiramer acetate related drug substance or drug product comprising the steps of:
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate drug related substance or drug product of step a); and
e) determining the level of expression of at least one gene disclosed herein or determining the level of biological activity of the cells of step c) as disclosed herein,
   thereby characterizing the glatiramer acetate related drug substance or drug product of step a).

This application claims priority of U.S. Provisional Application Nos. 61/819,481, filed May 3, 2013 and 61/749,228, filed Jan. 4, 2013, the contents of each of which are hereby incorporated by reference in their entireties.

Throughout this application various publications are referenced by numerical identifiers in parentheses. Full citations of these references can be found following the Examples. The disclosures of these publications in their entireties are hereby incorporated by reference into this application in order to more fully describe the state of the art to which this invention pertains.

Multiple sclerosis (MS) is a chronic, debilitating autoimmune disease of the central nervous system (CNS) with either relapsing-remitting (RR) or progressive course leading to neurologic deterioration and disability. At time of initial diagnosis, RRMS is the most common form of the disease (1) which is characterized by unpredictable acute episodes of neurological dysfunction (relapses), followed by variable recovery and periods of clinical stability. The vast majority of RRMS patients eventually develop secondary progressive (SP) disease with or without superimposed relapses. Around 15% of patients develop a sustained deterioration of their neurological function from the beginning; this form is called primary progressive (PP) MS. Patients who have experienced a single clinical event (Clinically Isolated Syndrome or “CIS”) and who show lesion dissemination on subsequent magnetic resonance imaging (MRI) scans according to McDonald's criteria, are also considered as having relapsing MS. (2)

With a prevalence that varies considerably around the world, MS is the most common cause of chronic neurological disability in young adults. (3, 4) Anderson et al. estimated that there were about 350,000 physician-diagnosed patients with MS in the United States in 1990 (approx. 140 per 100,000 population). (5) It is estimated that about 2.5 million individuals are affected worldwide. (6) In general, there has been a trend toward an increasing prevalence and incidence of MS worldwide, but the reasons for this trend are not fully understood. (5)

Current therapeutic approaches consist of i) symptomatic treatment ii) treatment of acute relapses with corticosteroids and iii) treatment aimed to modify the course of the disease. Currently approved therapies target the inflammatory processes of the disease. Most of them are considered to act as immunomodulators but their mechanisms of action have not been completely elucidated. Immunosuppressants or cytotoxic agents are also used in some patients after failure of conventional therapies. Several medications have been approved and clinically ascertained as efficacious for the treatment of RR-MS; including BETASERON®, AVONEX® and REBIF®, which are derivatives of the cytokine interferon beta (IFNB), whose mechanism of action in MS is generally attributed to its immunomodulatory effects, antagonizing pro-inflammatory reactions and inducing suppressor cells. (7) Other approved drugs for the treatment of MS include Mitoxantrone and Natalizumab.

Copaxone® (Teva Pharmaceutical Industries Ltd.) is a glatiramer acetate drug product approved for treatment of patients with relapsing-remitting multiple sclerosis (RRMS) and clinically isolated syndrome (CIS) (8). Glatiramer acetate drug substance (GA), the active substance of Copaxone®, is a complex mixture of polypeptides and is the first member of the glatiramoid class; i.e., a complex mixture of synthetic polypeptides of varying sizes assembled from four naturally occurring amino acids: L-glutamic acid, L-alanine, L-lysine, and L-tyrosine, in a defined molar ratio (9).

GA elicits anti-inflammatory as well as neuroprotective effects in various animal models of chronic inflammatory and neurodegenerative diseases (10-14) and has been shown to be safe and effective in reducing relapses and delaying neurologic disability in MS patients following long-term treatment (15).

The mechanisms underlying GA therapeutic activity are not fully elucidated, but GA activity on immune cells has been well demonstrated. GA appears to act as an altered peptide ligand (APL) of encephalitogenic epitopes within myelin basic protein (MBP) (16) and demonstrates cross-reactivity with MBP at the humoral and cellular levels (17-23). The unique antigenic sequences of the GA polypeptide mixture compete with myelin antigens for binding to MHC class II molecules on antigen presenting cells (APCs) and presentation to the T cell receptor (TCR), resulting in the induction of anergy or deletion of autoreactive MBP-reactive T cells and proliferation of GA-reactive T cells. At initiation of Copaxone treatment, GA-reactive CD4+ T-cell lines from MS patients secrete both pro-inflammatory T helper type 1 (Th1) and anti-inflammatory Th2 cytokines (21, 24), but continued exposure to Copaxone induces a shift in GA-reactive T cells toward the Th2 phenotype (21, 23, 25-28).

Copaxone also increases the number and suppressive capacity of CD4+CD25+FOXP3+ regulatory T cells, which are functionally impaired in MS patients (29-31). Furthermore, treatment leads to antigen-nonspecific modulation of APC function. Copaxone treatment promotes development of anti-inflammatory type II monocytes characterized by an increase in interleukin (IL)-10 and transforming growth factor-beta (TGF-β) and decreased production of IL-12 and tumor necrosis factor (TNF) (32).

### SUMMARY OF THE INVENTION

High-throughput gene expression analysis was used to further characterize the functional pathways that are modulated by GA within immune cells. This technique facilitates investigation of thousands of genes and allows identification of a wide range of biological functions. Microarray gene expression analyses were conducted using GA-primed murine splenocytes reactivated ex vivo with GA or with a variant referred to as GA-Natco (Glatimer®, Natco Pharma, Ltd., Hyderabad, India). The transcriptional alteration of genes induced by GA or GA-Natco, were evaluated with respect to functional pathways that may be related to known mechanisms of GA activity. This sensitive high-throughput gene expression analysis sheds some light on the mode of action of GA and on differences between various glatiramoids that are otherwise difficult to detect.

The present invention provides a process for characterizing a glatiramer acetate related drug substance or drug product comprising the steps of
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate drug related substance or drug product of step a); and
e) determining the level of expression of at least one gene selected from the group consisting of genes regulated by glatiramer acetate drug substance or drug product in Gene Expression Omnibus accession number GSE40566; determining the level of expression of at least one gene selected from the group consisting of Ecm1, Pres1, Pdlim4, Gpr83, Ifng, I124, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tpsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of at least one gene selected from the group consisting of CD40, CD86, GATA3, HLA-DMA, HLA-DMB, ICOS, IFNG, IFNGR2, IL2, IL13, IL4, IL18, IL12RB1, IL17A, IL17F, IL18R1, IL2RA, IL2RG, IL4R, IL6R, TBX21, TGFBR2, TNF, FOXP3, IL10RB, KLRD1, CD69, LTB, CD83, PRF1, CAMK2D, LTA, FSCN1, TLR7, CSF2, CCR7, FASLG, IL1A, CCL5, CD8B, CXCL10, TLR2, CCL4, TLR7, IGHA1, IL24, SOCS1, OAS1, JAK1, PTPN2, IFITM1, IFI35, STAT2, BCL2, MVD, FDPS, SQLE, NSDHL, DHCR24, Acat2/Acat3, MSMO1, LSS, CYP51A1, NFKBIE, PIK3R1, PPP3CC, CD3D, IL2RB, PTEN, CD3G, ICOS, CAMK2D, NFAT5, LAT, ITK, H2-M2, FASLG, LIF, IGHA1, PRKACB, SGK1, MAPK11, TSC22D3, JUN, FKBP5, ADRB2, MAP3K1, MAPK12, POU2F1, SMARCA2, CDKN1A, TGFB3, HSP90AA1, DHCR24, CCR5, and CXCL9; determining the level of expression of at least one gene selected from the group consisting of Foxp3, Il2, Il1a, Il1b, C3, S100a8, S100a9, Cxcl2, Cxcl3, Ccl4, Ccl3 and Cd14; determining the level of expression of at least one gene selected from the group consisting of the genes presented in Table 8; determining the level of expression of at least one gene selected from the group consisting of the genes presented in Table 10; determining the level of expression of at least one gene selected from the group consisting of FoxP3, GPR83, CD14, TLR2, IFNG, CD40 and IL1B; determining the level of expression of at least one gene selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ cells, macrophage cells, monocyte cells stromal cells, multi-lineage progenitor cells, dendritic cells, fibroblastic reticular cells, fibroblasts and granulocytes,
   thereby characterizing the glatiramer acetate related drug substance or drug product of step a).

The present invention also provides a process for characterizing a glatiramer acetate related drug substance or drug product comprising the steps of:
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate related drug substance or drug product of step a); and
e) determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever,
   thereby characterizing the glatiramer acetate related drug substance or drug product of step a).

The present invention also provides a process for discriminating between glatiramer acetate related drug substances or drug products comprising the steps of:
i) characterizing two or more glatiramer acetate related drug substances or drug products according to a process of the present invention to obtain characteristics of each of the glatiramer acetate related drug substances or drug products; and
ii) comparing the characteristics of the glatiramer acetate related drug substances or drug products obtained in step i),
   thereby discriminating between the glatiramer acetate related drug substances or drug products.

The present invention also provides a process for producing a drug product comprising a glatiramer acetate related drug substance, the improvement comprising the steps of
i) characterizing the glatiramer acetate related drug substance according to a process of the present invention, wherein step e) comprises determining the level of expression of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, A1s2c1, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tpsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and;
ii) discarding the batch of the glatiramer acetate related drug substance as unacceptable for inclusion in the drug product if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tpsab1, Ccl4, Il2, Inhba, Cxcl1, Serpinb2, Upp1, Gpr109a, Gp38, Il1b, Cxcl2, Il1a, and Ccl3, is increased relative to a reference standard; discarding the batch of the glatiramer acetate related drug substance as unacceptable for inclusion in the drug product if the level of expression of a gene selected from the group consisting of the genes presented in Table 8 is not substantially identical to the level of expression of a reference standard; discarding the batch of the glatiramer acetate related drug substance as unacceptable for inclusion in the drug product if the level of expression of a gene selected from the group consisting of the genes presented in Table 10 is not substantially identical to the level of expression of a reference standard; discarding the batch of the glatiramer acetate related drug substance as unacceptable for inclusion in the drug product if the level of expression of a gene selected from the group consisting of GPR83, IFNG and Foxp3 is decreased or if the level of expression of a gene selected from the group consisting of CD14, CD40, TLR2 and IL1B is increased; discarding the batch of the glatiramer acetate related drug substance as unacceptable for inclusion in the drug product if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or discarding the batch of the glatiramer acetate related drug substance as unacceptable for inclusion in the drug product if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes.

The present invention also provides a process for producing a drug product comprising a glatiramer acetate related drug substance, the improvement comprising the steps of:
i) characterizing the glatiramer acetate related drug substance according to a process of the present invention, wherein step e) comprises determining the level of expression of at least one gene selected from the group consisting of Foxp3, Il2, Il1a, Il1b, C3, S100a8, S100a9, Cxcl2, Cxcl3, Ccl4, Ccl3 and Cd14;
ii) discarding the batch of the glatiramer acetate related drug substance as unacceptable for inclusion in the drug product if the level of expression of FoxP3 is decreased relative to a reference standard or if the level of expression of at least one gene selected from the group consisting of Il2, Il1a, Il1b, C3, S100a8, S100a9, Cxcl2, Cxcl3, Ccl4, Ccl3 and Cd14 is increased relative to a reference standard.

The present invention also provides a process for producing a drug product comprising a glatiramer acetate related drug substance, the improvement comprising the steps of:
i) characterizing the glatiramer acetate related drug substance according to a process of the present invention, wherein step e) comprises determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever;
ii) discarding the batch of the glatiramer acetate related drug substance as unacceptable for inclusion in the drug product if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard.

The present invention also provides a process for releasing a drug product comprising a glatiramer acetate related drug substance, the improvement comprising the steps of:
i) characterizing the glatiramer acetate related drug product according to a process of the present invention, wherein step e) comprises determining the level of expression of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583Hl4Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and;
ii) discarding the batch of the glatiramer acetate related drug product as unacceptable for release if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; discarding the batch of the glatiramer acetate related drug product as unacceptable for release if the level of expression of a gene selected from the group consisting of the genes presented in Table 8 is not substantially identical to the level of expression of a reference standard; discarding the batch of the glatiramer acetate related drug product as unacceptable for release if the level of expression of a gene selected from the group consisting of the genes presented in Table 10 is not substantially identical to the level of expression of a reference standard; discarding the batch of the glatiramer acetate related drug product as unacceptable for release if the level of expression of a gene selected from the group consisting of GPR83, IFNG and Foxp3 is decreased or if the level of expression of a gene selected from the group consisting of CD14, CD40, TLR2 and IL1B is increased; discarding the batch of the glatiramer acetate related drug product as unacceptable for release if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or discarding the batch of the glatiramer acetate related drug product as unacceptable for release if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes.

The present invention also provides a process for releasing a drug product comprising a glatiramer acetate related drug substance, the improvement comprising the steps of:
i) characterizing the glatiramer acetate related drug product according to a process of the present invention, wherein step e) comprises determining the level of expression of at least one gene selected from the group consisting of Foxp3, Il2, Il1a, Il1b, C3, S100a8, S100a9, Cxcl2, Cxcl3, Ccl4, Ccl3 and Cd14;
   ii) discarding the batch of the glatiramer acetate related drug product as unacceptable for release if the level of expression of FoxP3 is decreased relative to a reference standard or if the level of expression of at least one gene selected from the group consisting of Il2, Il1a, Il1b, C3, S100a8, S100a9, Cxcl2, Cxcl3, Ccl4, Ccl3 and Cd14 is increased relative to a reference standard.

The present invention also provides a process for releasing a drug product comprising a glatiramer acetate related drug substance, the improvement comprising the steps of:
i) characterizing the glatiramer acetate related drug product according to a process of the present invention, wherein step e) comprises determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever;
ii) discarding the batch of the glatiramer acetate related drug product as unacceptable for release if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard.

The present invention also provides a method of identifying suboptimal activity of a glatiramer acetate related drug substance or drug product comprising the steps of:
a) administering a glatiramer acetate related drug substance or drug product to a rodent;
b) determining the level of biological activity of the rodent selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever; and
c) identifying the glatiramer acetate related drug substance or drug product as causing a suboptimal activity if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard,
   thereby identifying suboptimal activity of the glatiramer acetate related drug substance or drug product.

The present invention also provides a method of identifying suboptimal activity of a glatiramer acetate related drug substance or drug product comprising the steps of:
a) administering a glatiramer acetate related drug substance or drug product to a rodent
b) determining the level of expression in the rodent of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp,

S100a8, S100a9, Clecsf9, Saa3, 5033414K04Rik, Slc7a11, S1pi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+CD8+ T cells, macrophage cells, monocyte cells stromal cells, multi-lineage progenitor cells, dendritic cells, fibroblastic reticular cells, fibroblasts and granulocytes; and
c) identifying the glatiramer acetate related drug substance or drug product as causing a suboptimal activity if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; identifying the glatiramer acetate related drug substance or drug product as causing a suboptimal activity if the level of expression of a gene selected from the group consisting of the genes presented in Table 8 is not substantially identical to the level of expression of a reference standard; identifying the glatiramer acetate related drug substance or drug product as causing a suboptimal activity if the level of expression of a gene selected from the group consisting of the genes presented in Table 10 is not substantially identical to the level of expression of a reference standard; identifying the glatiramer acetate related drug substance or drug product as causing a suboptimal activity if the level of expression of a gene selected from the group consisting of GPR83, IFNG and Foxp3 is decreased or if the level of expression of a gene selected from the group consisting of CD14, CD40, TLR2 and IL1B is increased; identifying the glatiramer acetate related drug substance or drug product as causing a suboptimal activity if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or identifying the glatiramer acetate related drug substance or drug product as causing a suboptimal activity if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes,
   thereby identifying suboptimal activity of the glatiramer acetate related drug substance or drug product.

### BRIEF DESCRIPTION OF THE DRAWINGS

FIG. 1: PCA of quantile-normalized, batch-corrected signals, colored by activation groups.

FIG. 2: Gene-wise hierarchical clustering of 1474 genes with FDR-adjusted p value<0.05 and fold change≧1.3 between GA and Medium-reactivated samples. The genes and gene symbols are listed in columns, and the samples, ordered by sample type, are listed in rows.

FIG. 3: Gene-wise hierarchical clustering of 98 genes with FDR-adjusted p value <0.05 and fold change ≧1.3 between GA-RS and 8 GA-Natco samples. The genes and gene symbols are listed in rows, and the samples, ordered by sample type, are listed in columns. The expression levels of Medium and GA-DP samples for these 98 genes are presented as well. Genes shown, from top to bottom, are: 6720418B01Rik, 5830496L11Rik, Cd8b1, Fcgrt, LOC385615, Scml4, Fscn1, Ctsg, Mpo, Mpo, Prtn3, Mpo, Lyzs, Emr1, Chi3l1, Anxa3, Hp, Lyz2, Lyz, Fer1l3, Sirpa, Cd63, Clec4n, Clec4d, EG433016, Stfa1, Stfa1, Chi3l3, Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Upp1, Upp1, Gpr109a, Gp38, Il1b, Cxcl2, Il1a, Cxcl2, Ccl3, Ecm1, Pres1, Pdlim4, Gpr83, Ifng, Ifng, Il24, LOC100046608, Gm590, Gpr114, Tmie, Rasgpr1, Myo6, Pfkp, Usp18, Arl4c, Als2cl, 2810410P22Rik, Arl5a, Gbp2, Rasgrp1, Ankrd37, Tpi1, 4930583H14Rik, Ifit3, Ifit3, LOC667370, Klhdc1, Cd247, Igfbp4, Oas2, Bcl11b, and Bcl11b.

FIG. 4: The biological impact of GA is significantly more consistent than that of other glatiramoids. Among probes with variability induced by activation, more than 4-fold higher probes had significant variation by F-test in other glatiramoid-activated samples when compared to GA activated samples (A). Defining tolerance as the percentage of samples with expression levels falling within the range between the maximum and minimum expression levels induced by reference standard for that probe, for any given tolerance threshold the number of probes failing to meet this this threshold is displayed for both the other glatiramoid and GA (B), showing that in almost all cases more probes fail to meet tolerance following induction by the other glatiramoid. For each individual other glatiramoid batch, the percentage of probes with significant differences in variability when compared to either GA or GA reference standard are plotted in C, along with the percentage of probes with differences in variability between GA and reference standard (dashed green line) for comparison. In each case the other glatiramoid batches have greater differences in variability.

FIG. 5: Plot of the coefficient of variation (CV) as a function of intensity for each of the probes when activated by other glatiramoid (black) and GA (red), showing the smaller range of CVs in GA and the wider range in other glatiramoids at any given intensity.

FIG. 6: GA induces Tregs more effectively than other glatiramoids. (A) GA induces significantly higher expression of FoxP3 than other glatiramoid. FoxP3 is a key marker of Tregs, and (B) another key Treg marker Gpr83 shows a similar pattern of expression. (C) Both FoxP3 and Gpr83 are low in the same samples as indicated by scatter plot, further strengthening the case that the other glatiramoid fails to induce a strong Treg response in some patients.

FIG. 7: The GSEA enrichment for the FoxP3 and Treg genes. (A) As further evidence of the difference in FoxP3 induction, GSEA analysis found a significantly stronger upregulation of FoxP3 target genes in GA-activated samples than in other glatiramoid-activated samples. (B) GSEA analysis also found a significant enrichment of Treg-specific genes among the genes with higher expression in GA than in other glatiramoid. NS=not significant.

FIG. 8: Cell-type specific differences in the biological impact of GA and other glatiramoids. The heat map depicts relative expression of specific genes in GA-activated samples and other glatiramoid-activated samples. Each of the rows within the Treg section represents a gene with a high cell-type specificity scores for Tregs, while each of the rows in the macrophages and monocyte sections represents genes with high cell-type specificity scores for each of those cell types. The associated gene lists appear as supplementary information. Overall, GA induces higher expression of Treg-associated genes than other glatiramoid, while other glatiramoid induces higher expression of macrophage and monocyte-associated genes than GA.

FIG. 9: Box plots of CD14 and TLR2, depicting the lower expression levels in GA and Reference compared to other glatiramoid. This is an additional way of visualizing the differences depicted by kernel density plots in FIG. 10A.

FIG. 10: Other glatiramoid's impact on monocytes may differ from GA's impact. (A) Other glatiramoid induces significantly higher expression of CD14 and TLR2, as determined by a Wilcoxon rank sum test and depicted as kernel density plots, which can be likened to a smoothed histogram. (B) CD14 and TLR2 expression are both unusually high in the same (mostly other glatiramoid) samples. (C) FoxP3 expression is unusually low in the sample samples in which CD14 expression is unusually high, suggesting that the other glatiramoid's different impact on monocytes may be related to its different impact on Tregs and consistent with literature suggesting that monocytes play a role in GA-induced FoxP3 expression. (D) FoxP3 expression is unusually low in the sample samples in which IL1B expression is unusually high, suggesting that the other glatiramoid's different impact on monocytes may be related to the differences between LPS-activated monocytes and T-cell contact activated monocytes, which have been described in the literature as having opposite impacts on IL1B production. (E) GSEA analysis found a significant enrichment of monocyte and macrophage-specific genes among the genes with higher expression in other glatiramoid than GA. NS=not significant.

FIG. 11: Scatter plots showing that the same other glatiramoid samples with unusually low FoxP3 expression also had unusually low IFNG expression, by two different probes of IFNG. Scatter plots illustrating that for two different probes of IFNG, GA and Reference standard upregulated IFNG to a greater extent than other glatiramoid did.

FIG. 12: Kernel density plot of CD40, illustrating the fact that this gene had higher expression in other glatiramoid-activated samples than in GA activated samples, consistent with the determination by the Wilcoxon rank-sum test and consistent with literature.

FIG. 13: Scatterplot illustrating the high degree of correlation between CD14 and IL1B, lending support to the hypothesis that the IL1B is expressed primarily by monocytes.

FIG. 14: GSEA analysis showing that genes with higher expression in other glatiramoid than medium are enriched in genes specific to CD16dim monocytes, while genes with higher expression in GA than medium are enriched in genes specific to CD16+monocytes.

FIG. 15: Flow chart of process for comparing an innovative medicine to a other glatiramoid, and model of key differences between GA and other glatiramoid (A) Overview of the methods for analyzing gene expression data to compare the immunological impact of GA to that of other glatiramoid. After processing, direct differences are identified by multiple parametric methods, non-parametric methods, as well as ANOVA-based pattern analysis, and variability analysis. The genes identified by these methods are analyzed using a variety of enrichment-based methods, which result in hypotheses that are then verified through additional methods. (B) The key hypotheses emerging from our studies involve the greater heterogeneity in the other glatiramoid's biological impact compared to GA's, and the fact that GA appears to more effectively upregulate FoxP3 expression and promote tolerance-inducing Tregs, while other glatiramoid appears to upregulated myeloid lineage cells such as monocytes and macrophages which may impair tolerance. Given these findings, it is reasonable to hypothesize that GA may suppress harmful cytotoxic cells more effectively than other glatiramoid, and this hypothesis warrants further investigation.

FIG. 16: Illustration of the tolerance method for comparing variability. The expression of genes following activation by GA and other glatiramoid are assessed to determine the percentage of samples following within a tolerance defined by the maximum and minimum expression levels induced by the reference standard (top and bottom of the red box for Gpr83, left and right sides of the red box for FoxP3).

### DETAILED DESCRIPTION OF THE INVENTION

### Embodiments of the Invention

The present invention provides a process for characterizing a glatiramer acetate related drug substance or drug product comprising the steps of:
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate drug related substance or drug product of step a); and
e) determining the level of expression of at least one gene selected from the group consisting of genes regulated by glatiramer acetate drug substance or drug product in Gene Expression Omnibus accession number GSE40566; determining the level of expression of at least one gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of at least one gene selected from the group consisting of CD40, CD86, GATA3, HLA-DMA, HLA-DMB, ICOS, IFNG, IFNGR2, IL2, IL13, IL4, IL18, IL12RB1, IL17A, IL17F, IL18R1, IL2RA, IL2RG, IL4R, IL6R, TBX21, TGFBR2, TNF, FOXP3, IL10RB, KLRD1, CD69, LTB, CD83, PRF1, CAMK2D, LTA, FSCN1, TLR7, CSF2, CCR7, FASLG, ILIA, CCL5, CD8B, CXCL10, TLR2, CCL4, TLR7, IGHA1, IL24, SOCS1, OAS1, JAK1, PTPN2, IFITM1, IFI35, STAT2, BCL2, MVD, FDPS, SQLE, NSDHL, DHCR24, Acat2/Acat3, MSMO1, LSS, CYP51A1, NFKBIE, PIK3R1, PPP3CC, CD3D, IL2RB, PTEN, CD3G, ICOS, CAMK2D, NFAT5, LAT, ITK, H2-M2, FASLG, LIF, IGHA1, PRKACB, SGK1, MAPK11, TSC22D3, JUN, FKBP5, ADRB2, MAP3K1, MAPK12, POU2F1, SMARCA2, CDKN1A, TGFB3, HSP90AA1, DHCR24, CCR5, and CXCL9; determining the level of expression of at least one gene selected from the group consisting of Foxp3, Il2, Il1a, Il1b, C3, S100a8, S100a9, Cxcl2, Cxcl3, Ccl4, Ccl3 and Cd14; determining the level of expression of at least one gene selected from the group consisting of the genes presented in Table 8; determining the level of expression of at least one gene selected from the group consisting of the genes presented in Table 10; determining the level of expression of at least one gene selected from the group consisting of FoxP3, GPR83, CD14, TLR2, IFNG, CD40 and IL1B; determining the level of expression of at least one gene selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes,
   thereby characterizing the glatiramer acetate related drug substance or drug product of step a).

The present invention also provides a process for characterizing a glatiramer acetate related drug substance or drug product comprising the steps of:
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate related drug substance or drug product of step a); and
e) determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever,
   thereby characterizing the glatiramer acetate related drug substance or drug product of step a).

The present invention also provides a process for discriminating between glatiramer acetate related drug substances or drug products comprising the steps of:
i) characterizing two or more glatiramer acetate related drug substances or drug products according to a process of the present invention to obtain characteristics of each of the glatiramer acetate related drug substances or drug products; and

ii) comparing the characteristics of the glatiramer acetate related drug substances or drug products obtained in step i),

thereby discriminating between the glatiramer acetate related drug substances or drug products.

In one or more embodiments of the present invention, the mammal is a rodent.

In one or more embodiments of the present invention, the culture of step c) is a primary culture.

In one or more embodiments of the present invention, the glatiramer acetate related drug substance or drug product of step a) is glatiramer acetate drug substance or drug product.

In one or more embodiments of the present invention, the glatiramer acetate related drug substance or drug product of step a) is a glatiramer acetate related drug substance or drug product other than glatiramer acetate drug substance or drug product.

In one or more embodiments of the present invention, the glatiramer acetate related drug substance or drug product of step b) is glatiramer acetate drug substance or drug product.

In one or more embodiments of the present invention, the glatiramer acetate related drug substance or drug product of step b) is a glatiramer acetate related drug substance or drug product other than glatiramer acetate drug substance or drug product.

In one or more embodiments of the present invention, the glatiramer acetate related drug substance or drug product of step b) is the same glatiramer acetate related drug substance or drug product of step a).

In one or more embodiments of the present invention, the glatiramer acetate related drug substance or drug product of step b) is a different glatiramer acetate related drug substance or drug product than the glatiramer acetate related drug substance or drug product of step a).

The present invention also provides a process for producing a drug product comprising a glatiramer acetate related drug substance, the improvement comprising the steps of:
i) characterizing the glatiramer acetate related drug substance according to a process of the present invention, wherein step e) comprises determining the level of expression of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tpsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and;
ii) discarding the batch of the glatiramer acetate related drug substance as unacceptable for inclusion in the drug product if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; discarding the batch of the glatiramer acetate related drug substance as unacceptable for inclusion in the drug product if the level of expression of a gene selected from the group consisting of the genes presented in Table 8 is not substantially identical to the level of expression of a reference standard; discarding the batch of the glatiramer acetate related drug substance as unacceptable for inclusion in the drug product if the level of expression of a gene selected from the group consisting of the genes presented in Table 10 is not substantially identical to the level of expression of a reference standard; discarding the batch of the glatiramer acetate related drug substance as unacceptable for inclusion in the drug product if the level of expression of a gene selected from the group consisting of GPR83, IFNG and Foxp3 is decreased or if the level of expression of a gene selected from the group consisting of CD14, CD40, TLR2 and IL1B is increased; discarding the batch of the glatiramer acetate related drug substance as unacceptable for inclusion in the drug product if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or discarding the batch of the glatiramer acetate related drug substance as unacceptable for inclusion in the drug product if gene set enrichment analysis indicates downregulation or a lack of upregulation for genes associated with at least one cell type selected from the group consisting of FoxP3+CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes.

The present invention also provides a process for producing a drug product comprising a glatiramer acetate related drug substance, the improvement comprising the steps of:
i) characterizing the glatiramer acetate related drug substance according to a process of the present invention, wherein step e) comprises determining the level of expression of at least one gene selected from the group consisting of Foxp3, Il2, Il1a, Il1b, C3, S100a8, S100a9, Cxcl2, Cxcl3, Ccl4, Ccl3 and Cd14;
ii) discarding the batch of the glatiramer acetate related drug substance as unacceptable for inclusion in the drug product if the level of expression of FoxP3 is decreased relative to a reference standard or if the level of expression of at least one gene selected from the group consisting of Il2, Il1a, Il1b, C3, S100a8, S100a9, Cxcl2, Cxcl3, Ccl4, Ccl3 and Cd14 is increased relative to a reference standard.

The present invention also provides a process for producing a drug product comprising a glatiramer acetate related drug substance, the improvement comprising the steps of:
i) characterizing the glatiramer acetate related drug substance according to a process of the present invention, wherein step e) comprises determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever;
ii) discarding the batch of the glatiramer acetate related drug substance as unacceptable for inclusion in the drug product if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard.

The present invention also provides a process for releasing a drug product comprising a glatiramer acetate related drug substance, the improvement comprising the steps of
i) characterizing the glatiramer acetate related drug product according to a process of the present invention, wherein step e) comprises determining the level of expression of one or more genes selected from the group consisting of Ecm1, Pres1, Pdlim4, Gpr83, Ifng, Il4, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Ills, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and;
ii) discarding the batch of the glatiramer acetate related drug product as unacceptable for release if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; discarding the batch of the glatiramer acetate related drug product as unacceptable for release if the level of expression of a gene selected from the group consisting of the genes presented in Table 8 is not substantially identical to the level of expression of a reference standard; discarding the batch of the glatiramer acetate related drug product as unacceptable for release if the level of expression of a gene selected from the group consisting of the genes presented in Table 10 is not substantially identical to the level of expression of a reference standard; discarding the batch of the glatiramer acetate related drug product as unacceptable for release if the level of expression of a gene selected from the group consisting of GPR83, IFNG and Foxp3+ is decreased or if the level of expression of a gene selected from the group consisting of CD14, CD40, TLR2 and IL1B is increased; discarding the batch of the glatiramer acetate related drug product as unacceptable for release if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or discarding the batch of the glatiramer acetate related drug product as unacceptable for release if gene set enrichment analysis indicates downregulation or a lack of upregulation for genes associated with at least one cell type selected from the group consisting of FoxP3+ CD4+T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes.

The present invention also provides a process for releasing a drug product comprising a glatiramer acetate related drug substance, the improvement comprising the steps of:
i) characterizing the glatiramer acetate related drug product according to a process of the present invention, wherein step e) comprises determining the level of expression of at least one gene selected from the group consisting of Foxp3, Il2, Il1a, Il1b, C3, S100a8, S100a9, Cxcl2, Cxcl3, Ccl4, Ccl3 and Cd14;
ii) discarding the batch of the glatiramer acetate related drug product as unacceptable for release if the level of expression of FoxP3 is decreased relative to a reference standard or if the level of expression of at least one gene selected from the group consisting of Il2, Il1a, Il1b, C3, S100a8, S100a9, Cxcl2, Cxcl3, Ccl4, Ccl3 and Cd14 is increased relative to a reference standard.

The present invention also provides a process for releasing a drug product comprising a glatiramer acetate related drug substance, the improvement comprising the steps of:
i) characterizing the glatiramer acetate related drug product according to a process of the present invention, wherein step e) comprises determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever;
ii) discarding the batch of the glatiramer acetate related drug product as unacceptable for release if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard.

The present invention also provides a method of identifying suboptimal activity of a glatiramer acetate related drug substance or drug product comprising the steps of:
a) administering a glatiramer acetate related drug substance or drug product to a rodent;
b) determining the level of biological activity of the rodent selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever; and
c) identifying the glatiramer acetate related drug substance or drug product as causing a suboptimal activity if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard,
   thereby identifying suboptimal activity of the glatiramer acetate related drug substance or drug product.

The present invention also provides a method of identifying suboptimal activity of a glatiramer acetate related drug substance or drug product comprising the steps of:
a) administering a glatiramer acetate related drug substance or drug product to a rodent
b) determining the level of expression in the rodent of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and
c) identifying the glatiramer acetate related drug substance or drug product as causing a suboptimal activity if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2c1, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; identifying the glatiramer acetate related drug substance or drug product as causing a suboptimal activity if the level of expression of a gene selected from the group consisting of the genes presented in Table 8 is not substantially identical to the level of expression of a reference standard; identifying the glatiramer acetate related drug substance or drug product as causing a suboptimal activity if the level of expression of a gene selected from the group consisting of the genes presented in Table 10 is not substantially identical to the level of expression of a reference standard; identifying the glatiramer acetate related drug substance or drug product as causing a suboptimal activity if the level of expression of a gene selected from the group consisting of GPR83, IFNG and Foxp3 is decreased or if the level of expression of a gene selected from the group consisting of CD14, CD40, TLR2 and IL1B is increased; identifying the glatiramer acetate related drug substance or drug product as causing a suboptimal activity if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or identifying the glatiramer acetate related drug substance or drug product as causing a suboptimal activity if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes,
   thereby identifying suboptimal activity of the glatiramer acetate related drug substance or drug product.

In one or more embodiments of the present invention, the level of expression is determined in the blood.

In one or more embodiments of the present invention, the level of expression is determined in PBMCs.

In one or more embodiments of the present invention, the reference standard is the level of expression prior to administration of the glatiramer acetate related drug substance or drug product.

In one or more embodiments of the present invention, the reference standard is the level of expression after administration of glatiramer acetate drug substance or drug product.

In one or more embodiments of the present invention, the rodent is a mouse.

In one or more embodiments of the present invention, the mouse is a female (SJL×BALB/C) F1 mouse.

In one or more embodiments of the present invention, the mouse is about 8 to about 12 weeks old.

In one or more embodiments of the present invention, the primary culture is a culture of spleen cells.

In one or more embodiments of the present invention, the primary culture is a culture of lymph node cells.

In one or more embodiments of the present invention, the primary culture of spleen cells is prepared about 3 days after immunization.

In one or more embodiments of the present invention, the incubation of step d) is for about 24 hours.

In one or more embodiments of the present invention, the glatiramer acetate related drug substance is a glatiramoid or wherein the glatiramer acetate related drug product comprises a glatiramoid.

In one or more embodiments of the present invention, the glatiramer acetate related drug substance is a glatiramoid other than glatiramer acetate drug substance or wherein the glatiramer acetate related drug product comprises a glatiramoid other than glatiramer acetate drug substance.

In one or more embodiments of the present invention the process or method comprises the step of determining the level of expression of at least one gene selected from the group consisting of Ecm1, Pres1, Pdlim4, Gpr83, Ifng, Il24, LOC100046608, Gm590, Gpr114, Tmie, Rasgrp1, Myo6, Pfkp, Uspl8, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4.

In one or more embodiments of the present invention the process or method comprises the step of determining the level of expression of at least one gene selected from the group consisting of Foxp3, Il2, Il1a, Il1b, C3, S100a8, S100a9, Cxcl2, Cxcl3, Ccl4, Ccl3 and Cd14.

In one or more embodiments of the present invention the process or method comprises the step of determining the level of expression of at least one gene selected from the group consisting of genes regulated by glatiramer acetate drug substance or drug product in

Gene Expression Omnibus accession number GSE40566.

In one or more embodiments of the present invention the process or method comprises the step of determining the level of expression of at least one gene selected from the group consisting of CD40, CD86, GATA3, HLA-DMA, HLA-DMB, ICOS, IFNG, IFNGR2, IL2, IL13, IL4, IL18, IL12RB1, IL17A, IL17F, IL18R1, IL2RA, IL2RG, IL4R, IL6R, TBX21, TGFBR2, TNF, FOXP3, IL10RB, KLRD1, CD69, LTB, CD83, PRF1, CAMK2D, LTA, FSCN1, TLR7, CSF2, CCR7, FASLG, IL1A, CCL5, CD8B, CXCL10, TLR2, CCL4, TLR7, IGHA1, IL24, SOCS1, OAS1, JAK1, PTPN2, IFITM1, IFI35, STAT2, BCL2, MVD, FDPS, SQLE, NSDHL, DHCR24, Acat2/Acat3, MSMO1, LSS, CYP51A1, NFKBIE, PIK3R1, PPP3CC, CD3D, IL2RB, PTEN, CD3G, ICOS, CAMK2D, NFAT5, LAT, ITK, H2-M2, FASLG, LIF, IGHA1, PRKACB, SGK1, MAPK11, TSC22D3, JUN, FKBP5, ADRB2, MAP3K1, MAPK12, POU2F1, SMARCA2, CDKN1A, TGFB3, HSP90AA1, DHCR24, CCR5, and CXCL9.

In one or more embodiments of the present invention the process or method comprises the step of determining the level of expression of at least one gene selected from the group consisting of the genes presented in Table 8.

In one or more embodiments of the present invention the process or method comprises the step of determining the level of expression of at least one gene selected from the group consisting of the genes presented in Table 10.

In one or more embodiments of the present invention the process or method comprises the step of determining the level of expression of at least one gene selected from the group consisting of FoxP3, GPR83, CD14, TLR2, IFNG, CD40 and IL1B.

In one or more embodiments of the present invention the process or method comprises the step of determining the level of expression of at least one gene selected from the group consisting of the genes presented in Table 12.

In one or more embodiments of the present invention the process or method comprises the step of determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes.

In one or more embodiments of the present invention, determining gene set enrichment analysis comprises the step of evaluating the level of expression of at least one gene selected from the group consisting of genes present in one or more of the rank list files presented in Table 11.

In one or more embodiments of the present invention, the reference standard is medium.

Definitions

As used herein, a “naÏve subject” is a subject that has not been treated with any multiple sclerosis drug.

As used herein, a “glatiramoid naïve subject” is a subject that has not been treated with any glatiramoid drug. A glatiramoid naÏve subject could have been treated with another multiple sclerosis drug.

As used herein, “in the blood of the subject” is represented by PBMCs, lymphocytes, monocytes, macrophages, basophils, dendritic cells or other cells derived from the subject's blood.

As used herein, a “reference standard” is a sample or value which serves as a point of comparison for another sample or value which differs from the reference standard with respect to one or more variables. With specific regard to a subject, a “reference standard” is a value or range of values that characterizes a defined population in a defined state of health. For example, a reference standard can characterize a healthy subject or a subject afflicted with multiple sclerosis, and when the subject is afflicted with multiple sclerosis the subject can be naïve or having received glatiramer acetate drug substance.

As used herein, the term “glatiramer acetate related drug substance” (GARDS) is intended to include any polypeptide that is able to compete with myelin basic protein on MHC class II in antigen presentation. Glatiramer acetate related substances include polypeptides with a predetermined sequence as well as mixtures of polypeptides assembled from the four amino acids glutamic acid (E), alanine (A), lysine (K), and tyrosine (Y); from any three of the amino acids Y, E, A and K, i.e. YAK, YEK, YEA or EAK; or from three of the amino acids Y, E, A and K and a fourth amino acid. Examples of glatiramer acetate related substances are disclosed in U.S. Pat. Nos. 6,514,938 A1, 7,299,172 B2, 7,560,100 and 7,655,221 B2 and U.S. Patent Application Publication No. US 2009-0191173 A1, the disclosures of which are hereby incorporated by reference in their entireties. Glatiramer acetate related substances include glatiramoids and glatiramer acetate drug substance.

As used herein, a “glatiramer acetate related drug product” (GARDP) contains a glatiramer acetate related drug substance.

As used herein, a “glatiramer acetate related drug substance or drug product” is a glatiramer acetate related drug substance or a glatiramer acetate related drug product.

As used herein a “glatiramoid” is a complex mixture of synthetic proteins and polypeptides of varying sizes assembled from four naturally occurring amino acids: L-glutamic acid, L-alanine, L-lysine, and L-tyrosine, in a defined molar ratio. Examples of glatiramoids include glatiramer acetate drug substance (e.g. Copaxone®) as well as glatiramoids other than Copaxone, e.g. GA-Natco.

As used herein “glatiramer acetate drug substance” (GADS) is glatiramer acetate produced by Teva Pharmaceutical Industries, Ltd. and is the active ingredient in a glatiramer acetate drug product.

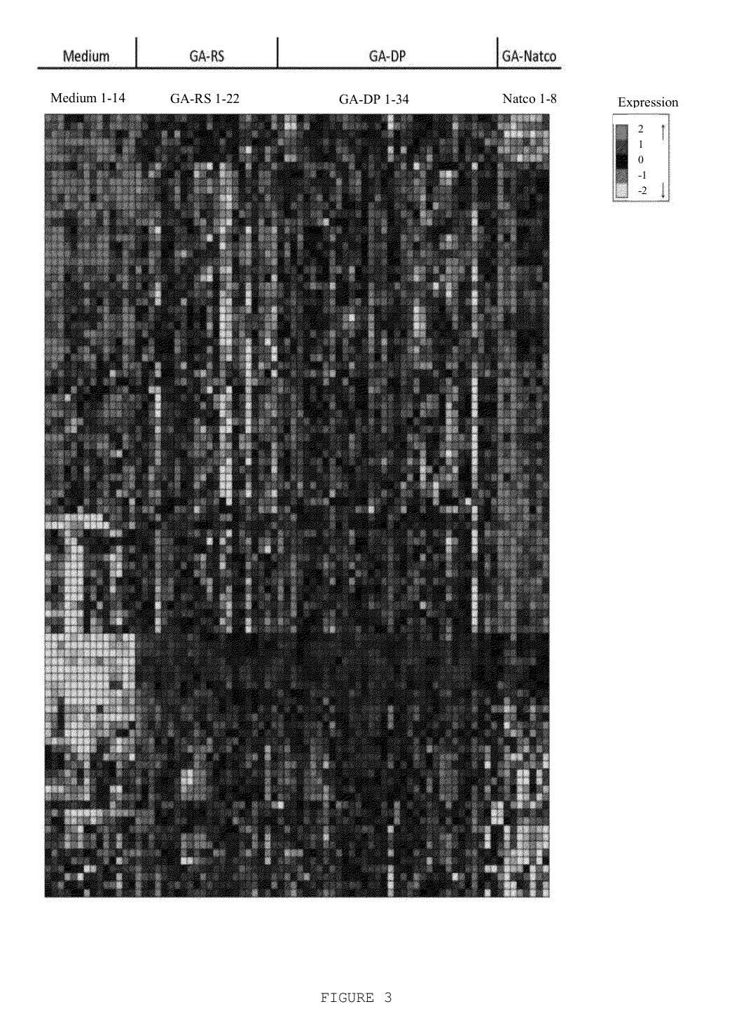

As used herein, a “glatiramer acetate drug product” (GAPD) contains a glatiramer acetate drug substance produced by Teva Pharmaceutical Industries, Ltd. which consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively, and has an average molecular weight of 5,000-9,000 daltons. (8) A glatiramer acetate drug product as well as a glatiramer acetate drug substance cause the response shown in FIG. 2 when tested according to Examples 1 and 2. Copaxone® is a glatiramer acetate drug product.

As used herein, a “glatiramer acetate drug substance or drug product” is a glatiramer acetate drug substance or a glatiramer acetate drug product.

As used herein a “glatiramer acetate reference standard” is or contains the drug substance found in a glatiramer acetate drug product. Examples of glatiramer acetate reference standards include the glatiramer acetate reference standards of Example 2.

As used herein “suboptimal activity” refers to a negative response or to a response which is less than the response to glatiramer acetate drug substance or glatiramer acetate drug product produced by Teva Pharmaceutical Industries, Ltd.

As used herein, “release” of a drug product refers to making the product available to consumers.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

Experimental Details

Methods

Mice

All experimental procedures conformed to accepted ethical standards for use of animals in research and were in accordance with Committee for the Care and Use of Experimental Animals guidelines and approved by the Teva Institutional Animal Care and Use Committee. For these experiments, we purchased 8- to 12-week old female (Balb/c×SJL)F1 mice (Harlan, Israel).

Preparation of Mouse Spleen Cell Cultures

All experimental procedures conformed to accepted ethical standards for use of animals in research and were in accordance with Committee for the Care and Use of Experimental Animals guidelines and approved by the Teva Institutional Animal Care and Use Committee. To stimulate induction of GA-reactive T cells, eight female mice 8- to 12-week-old F1 hybrid (SJL×BALB/C) (purchased from Harlan Biotech Israel, Rehovot, Israel) were injected with 100 μL of a 2.5 mg/mL solution of GA reference standard (GA-RS) in PBS (250 pg GA-RS per mouse). GA-RS (Teva) is a selected GA drug substance batch defined as the standard batch and used as a reference in Quality Control (QC) release tests of all Copaxone® batches. Mice were housed in individually ventilated cages for Three days after immunization, mice were sacrificed and their spleens were aseptically removed and placed on ice in RPMI 1640. Single cell suspension was prepared.

After red blood cells lysis, splenocytes were pulled and resuspended to a final concentration of 10×10⁶ cells/mL in defined cell culture media (DCCM1) (Biological Industries, Beit Haemek, Israel) (96.7% v/v) enriched with L-glutamine 2 mM (1% v/v), MEM 2 mM (1% v/v), sodium pyruvate 1 mM (1% v/v), antibiotic/antimycotic solution (0.2% v/v), and 2-mercaptoethanol (0.1% v/v).

In Vitro Cell Activation

Splenocytes were treated with either: A) GA, which included GA reference standard (GA-RS; 22 samples) and GA drug product (GA-DP, 34 samples from 30 batches) manufactured by Teva; and GA-Natco, which included 11 samples from 5 different batches of another glatiramoid manufactured by a company other than Teva.

Aqueous activator samples, mannitol (the non-active excipient in Copaxone® ), and Medium were added to 96-well tissue culture plates (3 wells per sample). Splenocytes (125 μL 10×10⁶ SPL cell/mL suspension) were added to the activator solutions and incubated for 24 hours at 37° C. Then, cells were washed, diluted with RNA stabilization solution (RNAlater® solution, Applied Biosystems), and stored at 4° C.

RNA Isolation and Microarray Expression Profiling

Extraction of total RNA from activated splenocytes was performed using PerfectPure RNA Cultures CEKK kit 50 (5Prime GmbH, Hamburg, Germany) and following the manufacturer's instructions. RNA quality was assessed by the absorbance ratio at 260/280 on and gel electrophoresis (Experion™, Bio-Rad, Hercules, Calif.). Total RNA extracted from samples was hybridized to Illumina Mouse WG-6_V2 microarray chips containing more than 45,200 transcripts.

Microarray hybridization, array scanning, and initial preprocessing were performed by BioRap (Technion, Haifa, Israel). Eight independent microarray experiments were conducted, each experiment containing one or more medium and GA samples, along with various GA-related Materials and Other Glatiramoids, samples

Data Analysis

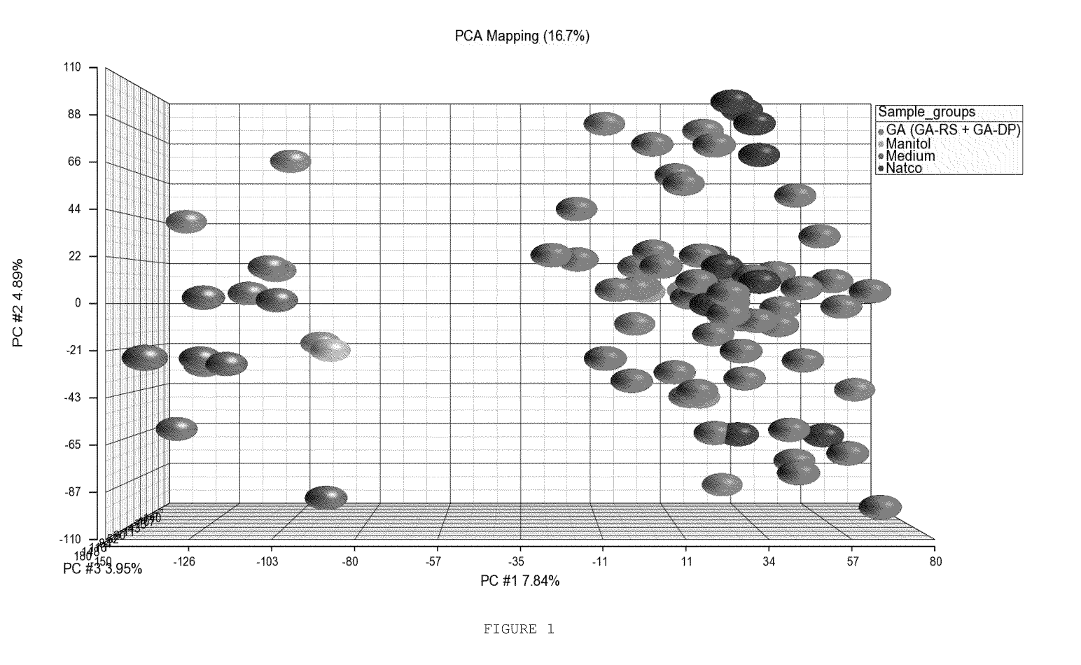

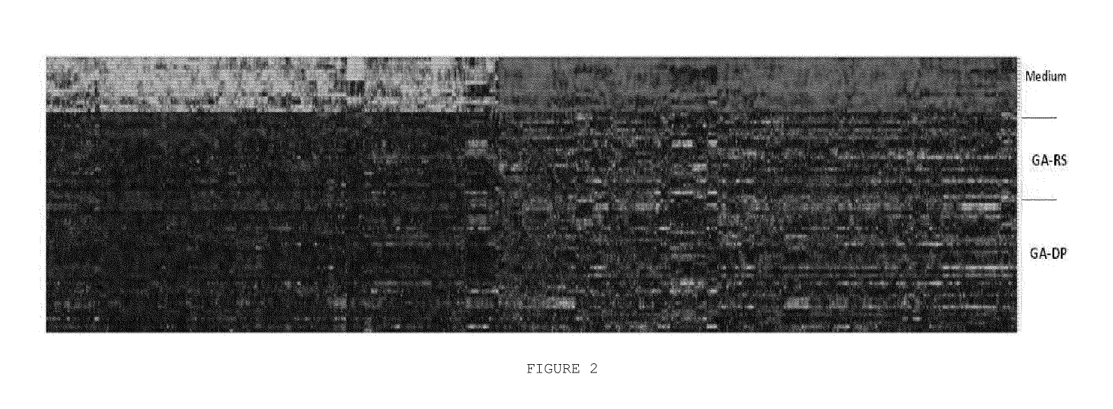

Initial bead-level data preprocessing was performed using Illumine BeadStudio software. The subsequent bioinformatics analyses were conducted in the Bioinformatics Core Facility of the National Institute for Biotechnology in the Negev, Ltd. (NIBN, Beer-Sheva, Israel). Summarization of bead-level data to gene-level signals was performed using the R package “beadarray” (45) after removal of outliers greater than 3 median absolute deviations (MADs). Between-sample quantile normalization was performed using Partek Genomics Suite (Partek, Inc., St. Louis, Missouri), followed by correction of experimental batch effects (i.e., related to differences in dates of RNA extraction and array processing) using the Partek Batch Remover™ tool. As a result, 34,666Expressed Genes with expression signals greater than 6 (in a log 2 scale) in at least one of the samples were retained for further analysis. Principal component analysis (PCA) of the achieved signals is shown in FIG. 1.

Statistical Tests for Identification of Differentially Expressed Genes

The 34,666 Expressed Genes were subjected to the following tests: (1) a t-test comparing the GA (i.e., GA-RS and GA-DP) and Medium; (2) a t-test comparing GA-Natco and Medium; and (3) a 1-Way ANOVA test comparing three sample groups: GA-RS, GA-DP, and GA-Natco. This test was run twice: once with all 11 GA-Natco samples, and once with only 8 of the GA-Natco samples. Each 1-Way ANOVA test included two pairwise comparisons (“contrasts”): GA-DP vs. GA-RS, and GA-Natco vs. GA-RS. Fold change values were presented in linear scale.

Hierarchical Clustering Analyses

Hierarchical clustering analyses were carried out in Expander (46) after standardizing the data so that the mean signal of each gene across the samples equaled zero and the standard deviation equaled one.

Microarray Data Sharing

Microarray data have been deposited in the Gene Expression Omnibus, (www.ncbi.nlm.nih.gov/geo), accession number GSE40566.

Functional Gene Expression Analysis

To identify the leading biological mechanisms associated with genes induced in splenocytes by glatiramoids, gene functional annotation, enrichment, and pathway analyses were performed by Ingenuity Pathways Analysis (IPA) Suite (www.ingenuity.com). The program integrates data from various experimental platforms and primary literature sources to provide insight into molecular interactions, cellular phenotypes, and disease processes, and then predicts downstream biological effects of gene transcription changes. IPA web software was also used to reconstruct regulatory genes and transcriptional factors functional networks. A significance score (using the right-tailed Fisher's exact test and expressed as a p value) for each process is calculated as the number of gene transcripts that participate in a network or pathway relative to the total number of these genes in all functional/pathway annotations stored in the IPA database. All p values were applied for multiple testing corrections using Benjamini-Hochberg FDR method with a cut off at p =0.05.

### EXAMPLE 1

Identification of Genes Regulated by GA and GA-Natco

Mice were immunized with GA reference standard (RS) and three days later, spleens were removed and cells extracted. Cultured splenocytes were reactivated ex-vivo with either medium, mannitol or glatiramoids (GA-RS, GA-DP or GA-Natco) for twenty four hours. RNA was extracted and full gene expression analysis was preformed.

Microarray Analysis

Principal component analysis (PCA) of the normalized gene expression signals showed two main clusters, with medium and mannitol samples in one cluster and glatiramoids (GA-RS, GA-DP and GA Natco) in the other cluster (FIG. 1).

A total of 1474 genes were up- or down-regulated by GA (i.e., GA reference standard, GA-RS and GA drug product, GA-DP) adjusted p value<0.05) with fold change of ≧1.3 compared to medium-treated samples (FIG. 2). Gene expression levels of cells activated by GA-RS and by GA-DP were statistically indistinguishable. The comparison between GA-Natco and medium indicated 1894 genes that were up- or down-regulated (FDR-adjusted p value <0.05, fold change of ≧1.3) with 1271 genes common to both the GA and GA-Natco signatures. When GA-Natco samples were directly compared with GA-RS samples, 75 genes had significantly different expression (FDR adjusted p-value <0.05). Gene expression patterns were inconsistent within the 11 GA-Natco samples (taken from 5 batches), separating into two separate clusters of 3 (2 batches) and 8 samples (of 3 batches). When the gene expression pattern of GA-RS and the 8 GA-Natco samples were compared, ninety eight genes had FDR adjusted p-value <0.05, with a fold change difference ≧1.3 (FIG. 3).

### EXAMPLE 2

Functional Analysis of Genes Differentially Expressed by GA vs. Medium

Functional analysis of the 1474 genes that were either up- or down-regulated following activation with GA samples indicated that among the most significantly influenced biological functions were those associated with increased proliferation and activation of immune cells, including T and B lymphocytes, stimulation and immune response of APCs, and differentiation of effector T-lymphocytes (Table 1). Simultaneously, functions related to quantity of cytotoxic T lymphocytes, and to development of hematopoietic progenitor cells, was down-regulated.

TABLE 1
Significantly enriched biological functions
in the GA gene expression signature

GA* vs. Medium

| Functional category | Response | p value^{†} |
|---|---|---|
| Proliferation of | Upregulated | 4.81E−38 |
| lymphocytes | | |
| Proliferation of immune | Upregulated | 2.86E−39 |
| cells | | |
| Proliferation of B | Upregulated | 9.66E−18 |
| lymphocytes | | |
| Proliferation of | Upregulated | 4.20E−11 |
| hematopoietic cell lines | | |
| Activation of | Upregulated | 1.88E−29 |
| lymphocytes | | |
| Activation of T | Upregulated | 7.03E−23 |
| lymphocytes | | |
| Activation of phagocytes | Upregulated | 4.96E−14 |
| Activation of | Upregulated | 4.24E−29 |
| mononuclear leukocytes | | |
| Activation of antigen | Upregulated | 1.05E−13 |
| presenting cells | | |
| Stimulation of antigen | Upregulated | 3.05E−07 |
| presenting cells | | |
| Immune response of | Upregulated | 1.57E−10 |
| antigen presenting cells | | |
| Stimulation of | Upregulated | 3.35E−08 |
| macrophages | | |
| Differentiation of | Upregulated | 1.99E−07 |
| effector T lymphocytes | | |
| Suppression of T | Upregulated | 5.93E−11 |
| lymphocytes | | |
| Development of | Down-regulated | 9.52E−08 |
| hematopoietic progenitor | | |
| cells | | |
| Quantity of cytotoxic T | Down-regulated | 5.42E−08 |
| cells | | |

*GA-RS and GA-DP
^{†}The significance value associated with a function is a measure of how likely it is that genes from the dataset participate in that function. The significance is expressed as a p value calculated using the right-tailed Fisher's exact test.

The significant canonical pathways associated with gene transcripts altered by GA, and their corresponding genes, can be found in Table 2. On this list, T helper cell differentiation was the most significant canonical pathway (p=4.97E-16), and transcriptional activation of this pathway (Table 3) is consistent with previously described mechanisms of GA immunomodulatory activity. GA induced Th1 cell activation by enhancing expression of genes encoding proinflammatory cytokines such as IFNγ, IL-2, and increasing expression of TBX21 transcription factor (Table 3). In addition, IL-17A and IL-17F, both related to the TH-17 pathway, were over-expressed in GA samples. Differentiation to the Th2 phenotype was evident by stimulation of genes encoding anti-inflammatory cytokines such as IL-4 and IL-13, as well as over-expression of IL-4R and the GATA family of transcription factors (GATA3), which stimulate IL-4 production (Table 3). IL18R expression was down-regulated by GA, which is also in line with Th2 phenotype (33). These findings are consistent with evidence from GA-treated MS patients showing that upon initiation of GA treatment, CD4+ T-cell lines secrete both pro-inflammatory Th1and anti-inflammatory Th2 cytokines (21, 24) and that continued exposure to GA induces a shift from a Th1-type cytokine profile to a Th2-type profile (21, 23, 27, 28). In that respect, our gene expression analysis captures the primary events taking place following initial activation of immune cells by GA, i.e. activation of both Th1 and Th2 pathways. FOXP3, an important transcription factor required for the maintenance of regulatory T cells, was over-expressed in GA samples, consistent with findings that GA increases the number and function of CD4+CD25+T regulatory cells in RRMS patients (30, 31, 34).

TABLE 2
Significantly Altered Genes and Most Significant Canonical
Pathways for GA Gene Expression Signature (GA reference standard
[GA-RS] and GA drug product [GA-DP] vs. medium).

| Canonical Pathways | p value | Genes |
|---|---|---|
| T Helper Cell | 4.97E−16 | IL12RB1, FOXP3, HLA-DMB, TBX21, |
| Differentiation | | IL18R1, TGFBR2, IL2, 1L17F, |
| | | IL4, HLA-DMA, IFNG, IL4R, IL2RG, |
| | | IL6R, IFNGR2, IL13, IL17A, ICOS, |
| | | IL18, CD40, IL10RB, CD86, IL2RA, |
| | | GATA3, TNF |
| Crosstalk between | 5.88E−11 | IFNG, IL2RG, KLRD1, CD69, LTB, |
| Dendritic Cells and | | CD83, IL2RB, IL18, PRF1, CAMK2D, |
| Natural Killer Cells | | CD40, IL2, LTA, FSCN1, TLR7, CD86, |
| | | CSF2, TNF, CCR7, FASLG, IL4 |
| Communication between | 4.41E−09 | IFNG, IL1A, CD83, CCL5, CD8B, |
| Innate and Adaptive Immune | | CXCL10, TLR2, IL18, CCL4, CD40, |
| Cells | | IL2, TLR7, CD86, IGHA1, CSF2, TNF, |
| | | CCR7, IL4 |
| Role of Cytokines in | 1.36E−06 | IFNG, IL1A, IL18, IL2, CSF2, IL13, |
| Mediating Communication | | IL17F, IL24, TNF, IL4, IL17A |
| between Immune Cells | | |
| Interferon Signaling | 5.58E−06 | SOCS1, IFNG, OAS1, JAK1, PTPN2, |
| | | IFNGR2, IFITM1, IFI35, STAT2, BCL2 |
| Super pathway of | 5.14E−06 | MVD, FDPS, SQLE, NSDHL, DHCR24, |
| Cholesterol Biosynthesis | | Acat2/Acat3, MSMO1, LSS, CYF51A1 |
| iCOS-iCOSL signaling in T | 5.16E−06 | HLA-DMA, IL2RG, NFKBIE, PIK3R1, |
| Helper Cells | | HLA-DMB, PPP3CC, CD3D, IL2RB, |
| | | PTEN, CD3G, ICOS, CAMK2D, NFAT5, |
| | | CD40, IL2, LAT, IL2RA, ITK |
| Differential Regulation of | 9.82E−06 | CCL4, CCL5, CSF2, IL13, IL17F, |
| Cytokine Production in | | TNF, IL17A |
| Macrophages and T Helper | | |
| Cells by IL-17A and IL-17F | | |
| Allograft Rejection | 9.88E−06 | IFNG, HLA-DMA, PRF1, CC40, H2-M2, |
| Signaling | | IL2, CD86, HLA-DMB, TNF, FASLG, |
| | | IL4 |
| Hematopoiesis from | 1.03E−05 | CD3G, IL1A, LIF, IL2, IGHA1, CSF2, |
| Pluripotent Stem Cells | | CD3D, CD8B, IL4 |
| Graft-versus-Host Disease | 1.42E−05 | IFNG, HLA-DMA, PRF1, IL1A, IL18, |
| Signaling | | IL2, CD86, HLA-DMB, TNF, FASLG |
| Glucocorticoid Receptor | 1.59E−05 | PRKACB, JAK1, SGK1, PIK3R1, |
| Signaling | | NFKBIE, CCL5, MAPK11, TSC22D3, |
| | | BCL2, TGFBR2, NFAT5, JUN, IL2, |
| | | FKBP5, ADRB2, IL4, IFNG, MAP3K1, |
| | | PPP3CC, MAPK12, IL13, CD3D, CD3G, |
| | | POU2F1, SMARCA2, CDKN1A, TGFB3, |
| | | HSP90AA1, CSF2, TNF |
| Cholesterol Biosythesis I | 1.72E−05 | SQLE, NSDHL, DHCR24, MSMO1, LSS, |
| | | CYP51A1 |
| Pathogenesis of | 2.24E−05 | CXCL10, CCR5, CXCL9, CCL4, CCL5 |
| Multiple Sclerosis | | |

TABLE 3
Significantly altered genes in the T helper cell differentiation
pathway expressed by splenocytes activated with GA

| | | p value | |
|---|---|---|---|
| | | GA* vs. | Fold |
| Symbol | Gene Name | Medium | Change |
| CD40 | CD40 molecule, TNF receptor | 1.68E−16 | 1.533 |
| | superfamily member 5 | | |
| CD86 | CD86 molecule | 5.45E−10 | 1.365 |
| GATA3 | GATA binding protein 3 | 2.69E−21 | 1.877 |
| HLA-DMA | major histocompatibility complex, | 5.03E−20 | 1.534 |
| | class II, DM alpha | | |
| HLA-DMB | major histocompatibility complex, | 9.87E−06 | 1.398 |
| | class II, DM beta | | |
| ICOS | inducible T-cell co-stimulator | 1.82E−17 | 2.164 |
| IFNG | interferon, gamma | 1.62E−41 | 16.847 |
| IFNGR2 | interferon gamma receptor 2 | 2.90E−05 | −1.333 |
| IL2 | interleukin 2 | 1.81E−15 | 2.074 |
| IL13 | interleukin 13 | 1.57E−13 | 1.330 |
| IL4 | interleukin 4 | 2.73E−30 | 4.428 |
| IL18 | interleukin 18 | 1.05E−16 | −1.383 |
| IL12RB1 | interleukin 12 receptor, beta 1 | 4.03E−24 | 2.270 |
| IL17A | interleukin 17A | 6.85E−08 | 1.451 |
| IL17F | interleukin 17F | 7.71E−12 | 2.365 |
| IL18R1 | interleukin 18 receptor 1 | 9.57E−21 | −1.726 |
| IL2RA | interleukin 2 receptor, alpha | 2.96E−28 | 2.457 |
| IL2RG | interleukin 2 receptor, gamma | 7.54E−08 | −1.386 |
| IL4R | interleukin 4 receptor | 5.56E−17 | 1.578 |
| IL6R | interleukin 6 receptor | 6.63E−10 | −1.545 |
| TBX21 | T-box 21 | 5.36E−24 | 2.654 |
| TGFBR2 | transforming growth factor, beta | 2.14E−16 | −1.436 |
| | receptor II | | |
| TNF | tumor necrosis factor | 4.79E−15 | 1.475 |
| FOXP3 | forkhead box P3 | 4.34E−20 | 1.406 |
| IL10RB | interleukin 10 receptor, beta | 6.16E−06 | −1.323 |

*GA-RS and GA-DP

### EXAMPLE 3

Functional Analysis of Genes Differentially Expressed by GA-Natco vs. Medium

Functional analysis of GA-Natco batches vs. medium indicated that, as in the GA signature, the most significantly influenced biological functions were associated with increased proliferation of lymphocytes (p=6.48E-35), immune cells (p=2.70E-35), B cells (p=2.74E-15), and hematopoietic cell lines (p=2.02E-10); activation of lymphocytes (6.03E-25), phagocytes (5.95E-07), and monocytes (p=2.25E-24); and stimulation of APCs (p=2.88E-6) and macrophages (P=1.87E-06).

As with GA, T helper cell differentiation was the most significant canonical pathway (p=1.37E-15) for the gene transcripts profile of GA-Natco compared with medium. The transcript signatures of GA-Natco appeared to have similar mechanisms within this pathway to those shown for GA, with a notable exception. FoxP3 was not overexpressed in splenocytes activated by GA-Natco, suggesting upregulation of CD4⁺CD25⁺FOXP3 Tregs could be different than GA.

Other important functional differences between GA and GA-Natco gene expression signatures are shown in Table 4. Induction with GA was characterized by upregulation of gene transcripts associated with activation of immune responses of APCs and functions related to differentiation of effector T-cells, with simultaneous suppression of T cells. Biological functions altered by GA-Natco were related to increased T lymphocyte proliferation and expansion of lymphocytes and monocytes. These findings suggest that, GA-Natco may be associated with decreased ability to activate APCs, inappropriate differentiation of effector T-cells, and less suppression of T cells. In addition, GA-Natco had more pro-inflammatory properties as demonstrated by increased expansion of lymphocytes and monocytes, and increased T-cell proliferation.

TABLE 4
Differences in enrichment of biological functions between
GA and GA-Natco gene expression signatures

| | GA* vs. Medium | | GA-Natco vs. Medium | |
|---|---|---|---|---|
| Functional category | Response | p value* | Response | p value |

Induced by GA and not by MA-Natco

| | | | | |
|---|---|---|---|---|
| Immune response of | Upregulated | 1.57E−10 | No Change | — |
| antigen presenting | | | | |
| cells | | | | |
| Differentiation of | Upregulated | 1.99E−07 | No change | — |
| effector | | | | |
| lymphocytes | | | | |
| Suppression of T | Upregulated | 5.93E−11 | No change | — |
| lymphocytes | | | | |

Induced by GA-Natco and not by GA

| | | | | |
|---|---|---|---|---|
| Expansion of | No change | — | Upregulated | 1.58E−15 |
| mononuclear | | | | |
| leukocytes | | | | |
| Proliferation of T | No change | — | upregulated | 1.49E−29 |
| lymphocytes | | | | |
| Expansion of | No change | — | Upregulated | 1.81E−15 |
| lymphocytes | | | | |
| Differentiation of | No change | — | Upregulated | 2.00E−06 |
| naive lymphocytes | | | | |

*GA-RS and GA-DP
^{†}The significance value associated with a function is a measure of how likely it is that genes from the dataset participate in that function. The significance is expressed as a p value that is calculated using the right-tailed Fisher's exact test.

### EXAMPLE 4

Functional Analysis of the 98 Genes Differentiating GA-Natco from GA-RS

As noted, direct comparison of 8 GA-Natco samples and GA-RS revealed 98 genes with fold change ≧1.3. These genes were associated with increased activation of functional processes of immune response, inflammatory response, adhesion of immune cells and stimulation of various cell migration and movement mechanisms (Table 5). In the inflammatory response function, inflammation was predicted to be increased in the GA-Natco samples (p=8.67E-19) given that 20 of the 30 differential transcripts were expressed in a direction consistent with active inflammatory response, including over-expression of IL2, IL1A, IL1B, C3, S100A8, and S100A9. Similarly, a prediction towards an increase in cell movement was also made (p=9.55E-15) since 29 of the 39 transcripts, including CXCL2, CXCL3 CCL4, were over-expressed in the GA-Natco samples. Interestingly, a network of inflammatory response genes associated with increased risk of fever, including IL1B, IL1A, CCL4, CCL3, CD14, was over-expressed as well.

TABLE 5
Increased activation of functional processes
in GA-Natco compared with GA-RS

| | | Predicted | Number of |
|---|---|---|---|
| | | activation | differentially |
| Functional category | p value | state | expressed genes |
| Inflammatory response | 8.67E−19 | Increased | 30 |
| Adhesion of immune | 7.57E−17 | Increased | 20 |
| cells | | | |
| Cell movement | 9.55E−15 | Increased | 39 |
| Migration of cells | 2.08E−15 | Increased | 38 |
| Chemotaxis of cells | 1.46E−14 | Increased | 22 |
| Cell movement of | 3.58E−15 | Increased | 22 |
| phagocytes | | | |
| Chemotaxis of | 1.18E−08 | Increased | 8 |
| monocytes | | | |
| Cell movement of | 1.09E−11 | Increased | 12 |
| monocytes | | | |
| Fever | 8.85E−09 | Increased | 6 |

### Discussion

### EXAMPLES 1-4

Glatiramer acetate drug substance (GA, Copaxone®) a mixture of polymers comprised of four amino acids, is an approved drug for treatment of relapsing-remitting multiple sclerosis (RRMS) and clinically isolated syndrome (CIS). GA mediates its activity by induction of GA-specific T cells that shift the T cell balance from a dominant pro-inflammatory phenotype (Th1/Th17), to an anti-inflammatory phenotype (Th2/Treg). In order to further characterize the functional pathways by which GA exerts its activity on immune cells, we used gene expression profiling on GA stimulated splenocytes. Mice were immunized with GA and after 3 days splenocytes were harvested and reactivated with GA ex vivo. Gene expression profile and pathway analysis were evaluated in reactivated splenocytes, showing a total of 1474 genes that were significantly up or down regulated by GA. The main functional pathways induced by GA were: increased proliferation and activation of immune cells including T and S lymphocytes, stimulation of antigen presenting cells, and differentiation of effector T lymphocytes. T helper cell differentiation was the most significant canonical pathway associated with gene transcripts altered by GA. Such expression patterns were not observed when another glatiramoid was used for cell activation. The GA induced pathways coincide with known mechanisms of GA activity in MS patients and further support the unique therapeutic effect of this drug.

We aimed to further investigate the mechanisms underlying GA therapeutic activity and to evaluate whether a closely related glatiramoid, GA-Natco, induces the same gene expression pattern as GA. Mice were immunized with GA-RS followed by exposure of excised splenocytes to ex-vivo activation with GA or GA-Natco. The most significant transcriptional changes induced by GA were related to T cell activation and differentiation, characterized by: a) upregulation of a mixture of pro- and anti-inflammatory cytokines, b) upregulation of CD4+ CD25+FOXP3 regulatory T cells, and c) suppression of T lymphocytes. These findings are consistent with previous publications suggesting that CD4+ T-cell lines, obtained from MS patients after short term GA treatment, secrete both proinflammatory Th1 (IL-2 and IFN-γ) and anti-inflammatory Th2 (IL-4, IL-5) cytokines (14, 28) while long term exposure to GA, results in a clear shift from the primarily Th1-type cytokine profile to a Th2-type profile (23, 27, 32, 36-40). GA treatment has also been shown to induce formation of CD4+CD25+ regulatory T cells by activation of FOXP3 expression (41) and to increase the number and suppressive capacity of CD4+CD25+FOXP3 and CD4+CD25+FOXP3+CD31+ regulatory T cells in MS patients (30, 31). In addition, our observations that the gene expressions profile of splenocytes treated with GA was functionally associated with induction of T cell suppression, is in line with GA observed effects in MS patients namely, enhanced suppressor activity of CD8+ T cells (42) and increased levels of CD3+CD8+CD28-suppressor T-cells (43).

GA also induced activation of APC related genes in the reactivated splenocytes. Studies in experimental autoimmune encephalomyelitis (EAE) models have shown that GA treatment activates and promotes development of APC's such as anti-inflammatory type II monocytes (32). These cells can promote T cell differentiation to Th2 cells and to CD4+CD25+FoxP3+ regulatory T cells and are considered important for the GA mechanism of action (32).

The specific gene expression pattern induced by GA was investigated in another study using freshly isolated PBMCs from RRMS patients before and after three months of daily GA treatment (44). In this study, GA treatment induced a differential expression of 480 genes. Some of these genes were related to cell proliferation and immune response mechanisms, a finding that coincides with our findings. However, other genes that were depicted at this study were related to antigen-activated apoptosis, adhesion mechanisms and MHC class-I antigen presentation (44). The variations between studies in gene expression patterns may be attributed to protocol differences, including the origin of the cells (human PBMCs vs. murine splenocytes), duration of in-vivo exposure to GA (3 months vs a few days) and the absence of an ex-vivo reactivation phase in the RRMS PBMCs study.

Ex vivo activation of GA-primed splenocytes with GA-DP samples produced statistically indistinguishable gene expression profiles when compared to splenocytes activated with GA-RS samples. In contrast, the gene expression pattern of the 11 GA-Natco samples was different in 75 genes and produced 2 distinct transcriptional profiles, one composed of three samples and the other of eight samples. Splenocyte activation by these eight GA-Natco samples showed significant differences in expression of 98 genes compared with splenocytes activated by GA.

When compared with GA, the eight GA-Natco samples had a gene expression signature consistent with a lack of transcriptional changes related to appropriate T cell differentiation; activation of

APCs, T-cell suppression, and activation of FOXP3 positive regulatory T cells. Moreover, differences between gene expression profiles induced by GA-Natco and GA-RS were associated with significant activation of inflammatory responses and increased inflammatory cell adhesion mechanisms.

In this study, mice were immunized with GA-RS and the transcriptional differences between GA and GA-Natco were determined only in the reactivation phase of GA-primed splenocytes; therefore, these results may not reflect all potential differences between GA and GA-Natco.

### EXAMPLE 5

Using glatiramer Acetate (GA) and another glatiramid (PG) as examples, we sought to develop a thorough set of computational methods to go beyond lists of differentially expressed genes, using transcriptional profiles to robustly compare the immunological impact of two medicines. We focused on (1) comparing the variability of the two drugs as measured in their transcriptional signatures for each drug batch; (2) characterizing the composition of the cell types modulated by each medicine; and (3) characterizing and explaining the immunomodulatory behaviors of the two drugs in the context of the genes they induce and suppress, as well as the immune cell types they modulate and the possible connections to clinical outcome.

### EXAMPLE 6

Variability Analysis: GA is Significantly more Consistent than PG

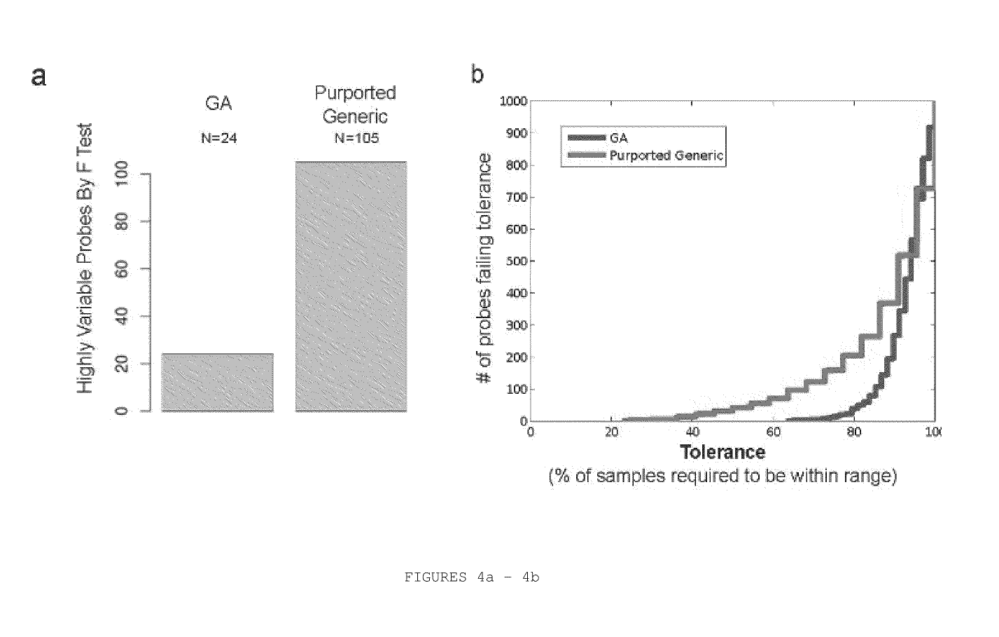

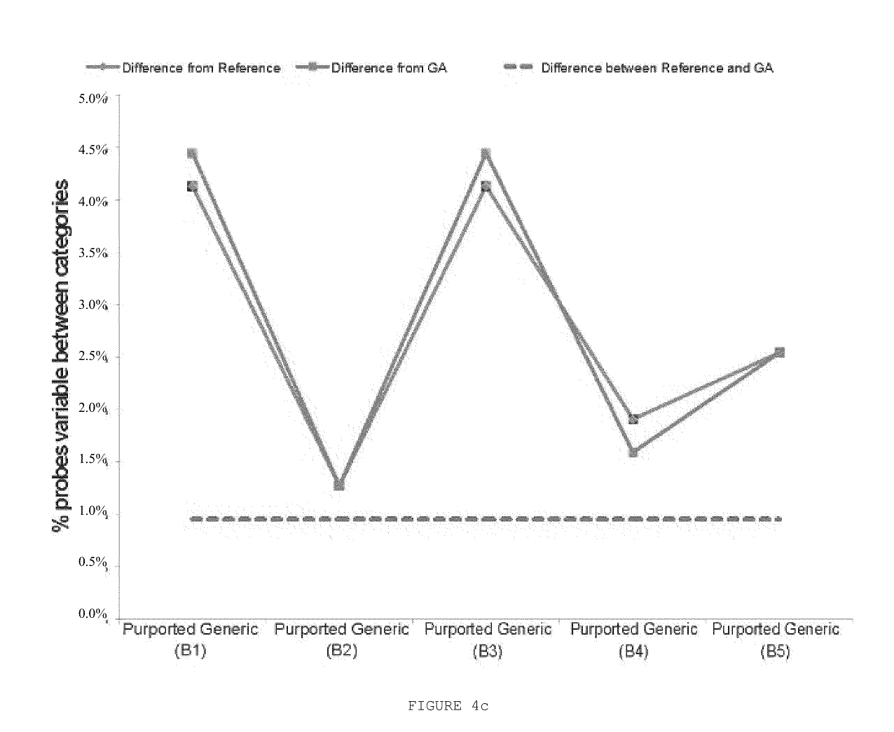

In comparing medicines produced by different manufacturing processes, it is important to assess if they are equally consistent in their biological impacts. We sought to examine differences in global variability across all relevant probes in order to address the question of whether the biological impact of PG was as consistent as GA. Defining relevant probes as those with variability induced specifically by activation (as opposed to experimental noise such as the variability seen in samples exposed only to medium), we found that 4-fold more probes had significantly higher variability in PG than in GA (FIG. 4A and Table 6).

TABLE 6
Highly variable probes significant by F-test in either GA or PG
The highly variable probes that were significant by F-test in either
GA or other glatiramoid and are depicted in FIG. 4A.

| Type | Probe | Gene |
|---|---|---|
| More variable in generic | ILMN_2927369 | KRT82 |
| More variable in generic | ILMN_1248780 | SPRR2B |
| More variable in generic | ILMN_1255612 | 9430016C22RIK |
| More variable in generic | ILMN_2465182 | XLR3B |
| More variable in generic | ILMN_1220648 | A630014I05RIK |
| More variable in generic | ILMN_2441895 | TYMS |
| More variable in generic | ILMN_1215680 | SAMD3 |
| More variable in generic | ILMN_2662283 | 2010007H12RIK |
| More variable in generic | ILMN_2730829 | TMUB1 |
| More variable in generic | ILMN_2534501 | LOC380738 |
| More variable in generic | ILMN_1229286 | 2410042D21RIK |
| More variable in generic | ILMN_2511527 | 9330133O14RIK |
| More variable in generic | ILMN_1228867 | A130082M07RIK |
| More variable in generic | ILMN_3009928 | 4930524E20RIK |
| More variable in generic | ILMN_2870788 | UPB1 |
| More variable in generic | ILMN_2941062 | SHROOM2 |
| More variable in generic | ILMN_2444847 | 4930521G14RIK |
| More variable in generic | ILMN_2805419 | OLFR885 |
| More variable in generic | ILMN_2825562 | CYBRD1 |
| More variable in generic | ILMN_2456193 | 6446580_669 |
| More variable in generic | ILMN_1225456 | CHRNA7 |
| More variable in generic | ILMN_2621217 | 5730508B09RIK |
| More variable in generic | ILMN_2495555 | MAPK8IP2 |
| More variable in generic | ILMN_2653725 | LOC100046802 |
| More variable in generic | ILMN_3161601 | SNCA |
| More variable in generic | ILMN_2436890 | DHRSX |
| More variable in generic | ILMN_1225663 | LOC384162 |
| More variable in generic | ILMN_1222683 | OLFR1441 |
| More variable in generic | ILMN_2691752 | OTX2 |
| More variable in generic | ILMN_2460292 | 8430427H17RIK |
| More variable in generic | ILMN_2706906 | LOC100047937 |
| More variable in generic | ILMN_2820893 | SELPLG |
| More variable in generic | ILMN_1233075 | ERCC2 |
| More variable in generic | ILMN_2733594 | D630004A14RIK |
| More variable in generic | ILMN_2768252 | MYL7 |
| More variable in generic | ILMN_1233191 | 5730422E09RIK |
| More variable in generic | ILMN_2935462 | SCN2B |
| More variable in generic | ILMN_2541335 | LOC384887 |
| More variable in generic | ILMN_2626648 | 41522 |
| More variable in generic | ILMN_2494532 | NRG1 |
| More variable in generic | ILMN_2682263 | KLHL28 |
| More variable in generic | ILMN_1378821 | control_ILMN_1378821 |
| More variable in generic | ILMN_1245775 | FUT9 |
| More variable in generic | ILMN_2752524 | PAQR9 |
| More variable in generic | ILMN_1234848 | TRIM2 |
| More variable in generic | ILMN_2564352 | SDHA |
| More variable in generic | ILMN_1260478 | HECW1 |
| More variable in generic | ILMN_2620284 | PHLDB1 |
| More variable in generic | ILMN_2741169 | CD8B1 |
| More variable in generic | ILMN_2728928 | FABP1 |
| More variable in generic | ILMN_2690135 | ACSL4 |
| More variable in generic | ILMN_2530757 | LOC210143 |
| More variable in generic | ILMN_1223894 | E530018B05RIK |
| More variable in generic | ILMN_2654541 | MED19 |
| More variable in generic | ILMN_2769762 | CTNNA2 |
| More variable in generic | ILMN_1378704 | control_ILMN_1378704 |
| More variable in generic | ILMN_1259595 | AI595406 |
| More variable in generic | ILMN_2635132 | FOXP3 |
| More variable in generic | ILMN_1250340 | A630055A13RIK |
| More variable in generic | ILMN_2524263 | ZFP251 |
| More variable in generic | ILMN_2690574 | FOXO3 |
| More variable in generic | ILMN_1239070 | MYO18B |
| More variable in generic | ILMN_2580818 | D930042N17RIK |
| More variable in generic | ILMN_2697415 | CD3D |
| More variable in generic | ILMN_1228620 | ZFP583 |
| More variable in generic | ILMN_2502290 | TRIM71 |
| More variable in generic | ILMN_2642321 | CEACAM13 |
| More variable in generic | ILMN_2661820 | AGXT2L1 |
| More variable in generic | ILMN_1244840 | 4833435K08RIK |
| More variable in generic | ILMN_1233941 | UNCX |
| More variable in generic | ILMN_1233843 | PTPN21 |
| More variable in generic | ILMN_1224922 | LRRN2 |
| More variable in generic | ILMN_2737523 | F2RL3 |
| More variable in generic | ILMN_2637897 | NLRX1 |
| More variable in generic | ILMN_1254631 | AI481316 |
| More variable in generic | ILMN_2498330 | LOC632997 |
| More variable in generic | ILMN_2769330 | CD6 |
| More variable in generic | ILMN_2666330 | OIT3 |
| More variable in generic | ILMN_2527328 | LOC381360 |
| More variable in generic | ILMN_2883666 | PIGN |
| More variable in generic | ILMN_1257579 | NUP210 |
| More variable in generic | ILMN_1235441 | STK2251 |
| More variable in generic | ILMN_2769325 | CD6 |
| More variable in generic | ILMN_3006990 | EG622339 |
| More variable in generic | ILMN_1216552 | CORO2B |
| More variable in generic | ILMN_2701383 | ANKS3 |
| More variable in generic | ILMN_1247936 | OLFR571 |
| More variable in generic | ILMN_2998230 | GALNS |
| More variable in generic | ILMN_2610965 | DAB2IP |
| More variable in generic | ILMN_1223081 | LOC241962 |
| More variable in generic | ILMN_2598271 | ASB17 |
| More variable in generic | ILMN_2979729 | OLFR1287 |
| More variable in generic | ILMN_1245710 | CCL2 |
| More variable in generic | ILMN_2705689 | GGA2 |
| More variable in generic | ILMN_1242912 | POF1B |
| More variable in generic | ILMN_2445249 | KRT36 |
| More variable in generic | ILMN_1235485 | LOC218501 |
| More variable in generic | ILMN_2964420 | LIPK |
| More variable in generic | ILMN_3104704 | KLRA4 |
| More variable in generic | ILMN_1246768 | A830026B15RIK |
| More variable in generic | ILMN_2923717 | KCNT1 |
| More variable in generic | ILMN_2798167 | BB287469 |
| More variable in generic | ILMN_1246408 | LOC100042427 |
| More variable in generic | ILMN_2749437 | RHOC |
| More variable in generic | ILMN_1243092 | EG546980 |
| More variable in GA | ILMN_1222230 | MEPE |
| More variable in GA | ILMN_1237927 | 8430407G10RIK |
| More variable in GA | ILMN_1380391 | control_ILMN_1380391 |
| More variable in GA | ILMN_1222029 | RTTN |
| More variable in GA | ILMN_1256073 | PXDN |
| More variable in GA | ILMN_1221957 | LOC637776 |
| More variable in GA | ILMN_1257904 | 2810403A07RIK |
| More variable in GA | ILMN_1244123 | SLC38A2 |
| More variable in GA | ILMN_2529810 | GPR113 |
| More variable in GA | ILMN_1231281 | LOC382900 |
| More variable in GA | ILMN_2756771 | GMFB |
| More variable in GA | ILMN_1260480 | 5033411B22RIK |
| More variable in GA | ILMN_1258114 | A930014PO8RIK |
| More variable in GA | ILMN_2524553 | 4930555L11RIK |
| More variable in GA | ILMN_2701797 | MEN1 |
| More variable in GA | ILMN_2499058 | UBE2I |
| More variable in GA | ILMN_2759380 | 2310010M20RIK |
| More variable in GA | ILMN_1244585 | RAP1GAP |
| More variable in GA | ILMN_2666205 | DPP10 |
| More variable in GA | ILMN_1240784 | LOC384938 |
| More variable in GA | 1770446 | 1770446 |
| More variable in GA | ILMN_1252393 | LOC635340 |
| More variable in GA | ILMN_2700224 | KTN1 |
| More variable in GA | ILMN_1217225 | CCDC91 |

As a second method of examining variability, we determined for each probe an acceptable range (i.e. between the minimum and maximum expression induced by GA reference standard). We determined the percentage of samples within this acceptable range for both GA and PG. We defined the maximum allowed percentage of samples with this range as the tolerance threshold, and found that for any given tolerance threshold PG almost always has more probes out of tolerance than GA (FIG. 4B). For instance, 158 probes for PG fail to meet a tolerance of 75% of samples within the range defined by the reference standard, while only 10 probes for GA fail to meet the same tolerance. The very worst PG probe has 5/22 samples within tolerance (22.7%), while the very worst GA probe has 43/68 (63%) of samples within tolerance.

As a third method of examining variability we calculated for each PG batch the percentage of differentially variable probes via F-test between that PG batch and the reference standard, as well as between that PG batch and GA. We then compared these values to the number of differentially variable probes between reference standard and GA, and found that each PG batch was more variable than GA, when both were compared to reference standard (FIG. 4C).

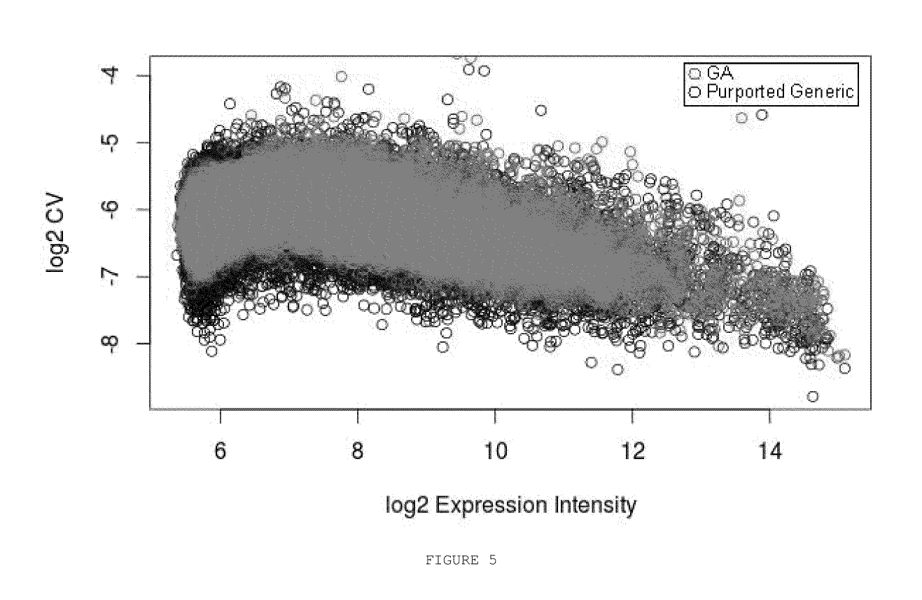

Finally, we examined the coefficient of variation (CV) across all probes in GA and in PG as a function of intensity, and found that there was a much narrower range of CV values present in GA than in PG at any given intensity (FIG. 5).

It is important not only to identify differences in variability, but also to explore the potential biological impact of these differences. Thus, we calculated for each probe the ratio of the variance in PG to the variance in GA. The highest ranked probe by variability in PG relative to its variability in GA was for FOXP3 (ILMN_{—}2635132, ratio 4.17, Table 7), the key marker of tolerance-inducing regulatory T cells (Tregs). The probe with the second highest ratio of variance in PG to variance in GA was for GPR83 (ILMN_{—}2707941, ratio 4.14), which is also an established Treg marker. (47)

TABLE 7
Ranked list of probes by ratio of the variance in other glatiramoid-activated
samples to the variance in GA-activated samples.

| Probe | Gene | Var(generic)/Var(GA) |
|---|---|---|
| ILMN_2635132 | FOXP3 | 4.169784637 |
| ILMN_2707941 | GPR83 | 4.141645173 |
| ILMN_2752702 | KLHL12 | 3.56612757 |
| ILMN_2741169 | CD8B1 | 3.455712087 |
| ILMN_2783997 | TRIM10 | 3.370881625 |
| ILMN_3161601 | SNCA | 3.340589295 |
| ILMN_2601946 | 5033414K04RIK | 3.297834654 |
| ILMN_1228867 | A130082M07RIK | 3.157262632 |
| ILMN_3136638 | SNCA | 3.14591837 |
| ILMN_2895284 | 2210023G05RIK | 3.126076675 |
| ILMN_2886128 | PCDHA10 | 3.085624289 |
| ILMN_1257368 | GM566 | 3.073208924 |
| ILMN_2635718 | BZRPL1 | 3.059590672 |
| ILMN_1239724 | PSMB1 | 3.031738703 |
| ILMN_1256142 | MARCK5 | 3.022926531 |
| ILMN_2690574 | FOXO3 | 2.972307993 |
| ILMN_1258520 | 4933421E18RIK | 2.903836286 |
| ILMN_2703061 | 2810408P10RIK | 2.867474892 |
| ILMN_2824625 | GM1604 | 2.825719679 |
| ILMN_1233843 | PTPN21 | 2.825301578 |
| ILMN_2620284 | PHLDB1 | 2.814372655 |
| ILMN_2745073 | LOC100047353 | 2.809357296 |
| ILMN_2749437 | RHOC | 2.773049302 |
| ILMN_1248780 | SPRR2B | 2.770363507 |
| ILMN_2703079 | PUNC | 2.754789803 |
| ILMN_1255719 | A930004L03RIK | 2.735701046 |
| ILMN_2733594 | D630004A14RIK | 2.712208469 |
| ILMN_1254631 | AI481316 | 2.710449678 |
| ILMN_2939277 | SNCG | 2.703626953 |
| ILMN_1225059 | SLC16A12 | 2.697244381 |
| ILMN_1217855 | NKG7 | 2.690910757 |
| ILMN_2753697 | CD2 | 2.674585226 |
| ILMN_1255513 | CDR2 | 2.671217726 |
| ILMN_2965737 | KLHL34 | 2.670969821 |
| ILMN_2524992 | EAR2 | 2.664017605 |
| ILMN_2498330 | LOC632997 | 2.64101161 |
| ILMN_1217629 | ITGAE | 2.640617288 |
| ILMN_2626648 | 41522 | 2.611392387 |
| ILMN_1215949 | 1700040F17RIK | 2.595459871 |
| ILMN_3114124 | HSD3B7 | 2.594406596 |
| ILMN_1216823 | UBR5 | 2.584859171 |
| ILMN_1222683 | OLFR1441 | 2.577789931 |
| ILMN_2606162 | PDLIM4 | 2.57565855 |
| ILMN_1223081 | LOC241962 | 2.54822461 |
| ILMN_2435477 | 5830406C15RIK | 2.537539864 |
| ILMN_2686825 | 5430427O19RIK | 2.536086924 |
| ILMN_1222512 | MFSD7B | 2.525669655 |
| ILMN_1248854 | ICK | 2.525643685 |
| ILMN_2705689 | GGA2 | 2.522100556 |
| ILMN_2826881 | MYBL2 | 2.522004768 |
| ILMN_1220648 | A630014I05RIK | 2.518845526 |
| ILMN_1378704 | control_ILMN_1378704 | 2.501520902 |
| ILMN_2715400 | TEF | 2.495018969 |
| ILMN_1257579 | NUP210 | 2.490045841 |
| ILMN_2598271 | ASB17 | 2.490015142 |
| ILMN_1225557 | E030019B06RIK | 2.484827893 |
| ILMN_2644350 | THY1 | 2.476638172 |
| ILMN_1216972 | CLEC4E | 2.47211937 |
| ILMN_2679094 | MAP3K7IP2 | 2.471543957 |
| ILMN_2820893 | SELPLG | 2.462368133 |
| ILMN_1223480 | D530031C13RIK | 2.461106681 |
| ILMN_2495703 | CLIP2 | 2.449801678 |
| ILMN_2679591 | ERAP1 | 2.449203035 |
| ILMN_2465182 | XLR3B | 2.447786159 |
| ILMN_1219011 | 3110040M04RIK | 2.445802442 |
| ILMN_2870696 | HFE | 2.434961339 |
| ILMN_3005873 | SORT1 | 2.431402316 |
| ILMN_2515285 | WASF3 | 2.431071739 |
| ILMN_2495555 | MAPK8IP2 | 2.429611427 |
| ILMN_2539917 | LOC384538 | 2.421951597 |
| ILMN_2757838 | IPPK | 2.416554447 |
| ILMN_3162301 | OTTMUSG00000015859 | 2.414336977 |
| ILMN_2923717 | KCNT1 | 2.414048507 |
| ILMN_2725054 | A930006J02RIK | 2.409634421 |
| ILMN_1230224 | DNAJC6 | 2.408553983 |
| ILMN_1225663 | LOC384162 | 2.408249488 |
| ILMN_1230818 | 9630015E22RIK | 2.406694567 |
| ILMN_2539295 | LOC621968 | 2.406675656 |
| ILMN_2950828 | C030018G13RIK | 2.405823034 |
| ILMN_2606792 | GBP4 | 2.404718824 |
| ILMN_1226665 | ISLR | 2.404321761 |
| ILMN_2769325 | CD6 | 2.398106015 |
| ILMN_1228696 | ADA | 2.39687535 |
| ILMN_2670368 | CAR5B | 2.393795851 |
| ILMN_2658878 | TG | 2.392922859 |
| ILMN_1242548 | HIF1A | 2.389939819 |
| ILMN_1254380 | DBR1 | 2.386871534 |
| ILMN_2446592 | RAD18 | 2.378012721 |
| ILMN_2425143 | MJ-500-31_190 | 2.377329303 |
| ILMN_2769330 | CD6 | 2.373941676 |
| ILMN_3155363 | PPL | 2.37383497 |
| ILMN_2454209 | TRBV6_AE000663_T_CELL_RECEPTOR_BETA_VARIABLE_6_11 | 2.372090892 |
| ILMN_1235584 | PLEKHA5 | 2.371229664 |
| ILMN_1218694 | C630017J20RIK | 2.370593123 |
| ILMN_2859348 | TAF9B | 2.367681107 |
| ILMN_1231458 | IQGAP1 | 2.366074862 |
| ILMN_2426480 | 3830612M24 | 2.365830273 |
| ILMN_2664439 | 9230109A22RIK | 2.3624709 |
| ILMN_2498851 | 2310075M15RIK | 2.359962418 |

### EXAMPLE 7

Comparing Impact on Key Immune System Genes: GA Induces Treg Markers FoxP3 and Gpr83 more Effectively than PG

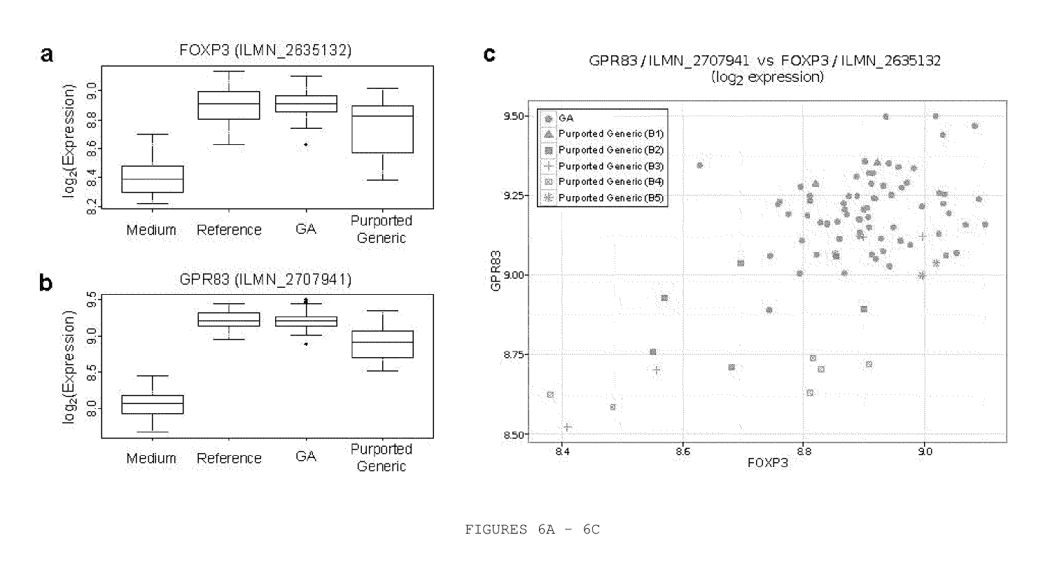

To systematically examine the differential expression of a particular gene in response to different medicines, we applied multiple methods including both parametric and non-parametric testing. Not only did GA induce FOXP3 expression more consistently than PG, but GA also induced significantly higher expression as determined by 4 parametric methods: ANOVA (adjusted p<1.37×10−3), LIMA with background subtraction (adjusted p<6.14×10−4), comparative marker selection using signal-to-noise (adjusted p<1.34×10−2 and t-test (adjusted p<2.12×10−2), and a non-parametric Wilcoxon rank-sum test (adjusted p<4.62×10−2 (FIG. 6A, Table 8).

Applying the same methods to GPR83, we found that GA induced significantly higher levels of expression than PG: ANOVA (adjusted p<4.75×10−8), LIMMA with background subtraction (adjusted p<8.67×10−10), comparative marker selection using signal-to-noise (adjusted p<1.34×10−2) and t-test (adjusted p<1.49×10−2), and a non-parametric Wilcoxon rank-sum test (adjusted p<3.45×10−4, FIG. 28). GPR83 is also in the top 20 probes by fold change from GA compared to PG (Table 8).

TABLE 8
Comparison of expression in GA to expression in GA for each
probe, including fold change, ANOVA, LIMMA with background
subtraction, comparative marker selection by signal-to-noise ratio,
comparative marker selection by t-test, and the Wilcoxon non-
parametric method.

| | | | Comparative Marker Selection | | |
|---|---|---|---|---|---|
| | | | T-test | | SNR |
| Probe | Gene | Fold Change | Nom. P. | Adj. P. | Nom. P. |
| ILMN_2685712 | IFNG | 1.41 | 0.000299498 | 0.014892062 | 0.000299498 |
| ILMN_2595857 | ANKRD37 | 1.36 | 0.000299498 | 0.014892062 | 0.000299498 |
| ILMN_2791459 | IFNG | 1.33 | 0.000299498 | 0.014892062 | 0.000299498 |
| ILMN_2675232 | KLK8 | 1.284884601 | 0.000299498 | 0.014892062 | 0.000299498 |
| ILMN_1220788 | GM590 | 1.284311292 | 0.000299498 | 0.014892062 | 0.000299498 |
| ILMN_1253828 | CD247 | 1.280944226 | 0.000299498 | 0.014892062 | 0.000299498 |
| ILMN_2856926 | GPR114 | 1.275591039 | 0.000299498 | 0.014892062 | 0.000299498 |
| ILMN_1218037 | TMIE | 1.25825101 | 0.000299498 | 0.014892062 | 0.000299498 |
| ILMN_2664929 | CD8B1 | 1.257683544 | 0.000299498 | 0.014892062 | 0.000299498 |
| ILMN_1240428 | SATB1 | 1.253362369 | 0.000299498 | 0.014892062 | 0.000299498 |
| ILMN_2741169 | CD8B1 | 1.24933849 | 0.000299498 | 0.014892062 | 0.000299498 |
| ILMN_1250001 | 4930583H14RIK | 1.24042799 | 0.000942625 | 0.026071536 | 0.000639896 |
| ILMN_2611022 | BCL11B | 1.239019043 | 0.000299498 | 0.014892062 | 0.000299498 |
| ILMN_2707941 | GPR83 | 1.238234714 | 0.000299498 | 0.014892062 | 0.000299498 |
| ILMN_2769772 | PIK3IP1 | 1.235639618 | 0.000299498 | 0.014892062 | 0.000299498 |
| ILMN_2775055 | LOC669166 | 1.234427524 | 0.000299498 | 0.014892062 | 0.000299498 |
| ILMN_3128992 | CD27 | 1.229968408 | 0.000299498 | 0.014892062 | 0.000299498 |
| ILMN_2416460 | TRBV8_AE000663_T_CELL_RECEPTOR_BETA_VARIA | 1.227075821 | 0.000299498 | 0.014892062 | 0.000299498 |
| ILMN_2670150 | OAS2 | 1.223676484 | 0.000299498 | 0.014892062 | 0.000299498 |
| ILMN_2454209 | TRBV6_AE000663_T_CELL_RECEPTOR_BETA_VARIA | 1.223323464 | 0.000299498 | 0.014892062 | 0.000299498 |
| ILMN_2960700 | PRF1 | 1.220037267 | 0.000299498 | 0.014892062 | 0.000299498 |
| ILMN_1229318 | PDK1 | 1.219922378 | 0.000299498 | 0.014892062 | 0.000299498 |
| ILMN_1233589 | CD27 | 1.219317837 | 0.000299498 | 0.014892062 | 0.000299498 |
| ILMN_2678521 | GPR146 | 1.215770831 | 0.000299498 | 0.014892062 | 0.000299498 |
| ILMN_1256701 | 2900016B01RIK | 1.215770789 | 0.000299498 | 0.014892062 | 0.000299498 |
| ILMN_1215167 | DDX5 | 1.214758445 | 0.000639896 | 0.021153497 | 0.000639896 |
| ILMN_3145331 | TMSB10 | 1.209579271 | 0.000299498 | 0.014892062 | 0.000299498 |
| ILMN_1252200 | A830055I09RIK | 1.206497974 | 0.000299498 | 0.014892062 | 0.000299498 |
| ILMN_2819380 | BC030476 | 1.206275831 | 0.000299498 | 0.014892062 | 0.000299498 |
| ILMN_2932359 | TPI1 | 1.204626968 | 0.000299498 | 0.014892062 | 0.000299498 |
| ILMN_2837493 | EG634650 | 1.204245484 | 0.000299498 | 0.014892062 | 0.000299498 |
| ILMN_1230137 | TPI1 | 1.204175926 | 0.000299498 | 0.014892062 | 0.000299498 |
| ILMN_2944666 | IFIT3 | 1.202746675 | 0.000299498 | 0.014892062 | 0.000299498 |
| ILMN_1217855 | NKG7 | 1.202473931 | 0.000639896 | 0.021153497 | 0.000639896 |
| ILMN_2699898 | ITGAE | 1.201910561 | 0.000299498 | 0.014892062 | 0.000299498 |
| ILMN_2516221 | TCRB-V8.2 | 1.20121668 | 0.000299498 | 0.014892062 | 0.000299498 |
| ILMN_2652867 | TRAT1 | 1.199470243 | 0.000299498 | 0.014892062 | 0.000299498 |
| ILMN_1229197 | OTTMUSG00000016644 | 1.198389483 | 0.000299498 | 0.014892062 | 0.000299498 |
| ILMN_2618148 | C330008K14RIK | 1.196159524 | 0.000299498 | 0.014892062 | 0.000299498 |
| ILMN_1214841 | IL24 | 1.195664917 | 0.000942625 | 0.026071536 | 0.000942625 |
| ILMN_1257496 | LOC665446 | 1.195420235 | 0.000299498 | 0.014892062 | 0.000299498 |
| ILMN_2421246 | TCRB-V8.3 | 1.195307771 | 0.000299498 | 0.014892062 | 0.000299498 |
| ILMN_2729252 | 5830431A10RIK | 1.195292385 | 0.000299498 | 0.014892062 | 0.000299498 |
| ILMN_2484932 | C030002B11RIK | 1.193361778 | 0.000299498 | 0.014892062 | 0.000299498 |
| ILMN_1221819 | FCGRT | 1.193070246 | 0.000299498 | 0.014892062 | 0.000299498 |
| ILMN_2981167 | IFIT2 | 1.192834198 | 0.000299498 | 0.014892062 | 0.000299498 |
| ILMN_2585233 | SELPL | 1.192768508 | 0.000299498 | 0.014892062 | 0.000299498 |
| ILMN_2697415 | CD3D | 1.191227881 | 0.000299498 | 0.014892062 | 0.000299498 |
| ILMN_1230345 | B830007D08RIK | 1.191119956 | 0.000299498 | 0.014892062 | 0.000299498 |
| ILMN_2811263 | ZXDA | 1.190267135 | 0.000299498 | 0.014892062 | 0.000639896 |
| ILMN_2560567 | A630006E02RIK | 1.189860092 | 0.000299498 | 0.014892062 | 0.000299498 |
| ILMN_2820893 | SELPLG | 1.188849449 | 0.000299498 | 0.014892062 | 0.000299498 |
| ILMN_1246609 | RASGRP1 | 1.18775349 | 0.000299498 | 0.014892062 | 0.000299498 |
| ILMN_2660551 | LAT | 1.187218377 | 0.000299498 | 0.014892062 | 0.000299498 |
| ILMN_2507761 | 7530404M11RIK | 1.186381811 | 0.000299498 | 0.014892062 | 0.000299498 |
| ILMN_2753697 | CD2 | 1.186334291 | 0.000299498 | 0.014892062 | 0.000299498 |
| ILMN_2601453 | A130092J06RIK | 1.186313516 | 0.000299498 | 0.014892062 | 0.000299498 |
| ILMN_1234565 | P4HA1 | 1.18626686 | 0.000299498 | 0.014892062 | 0.000299498 |
| ILMN_2459899 | ADAMTSL4 | 1.185956519 | 0.000299498 | 0.014892062 | 0.000299498 |
| ILMN_2761720 | LOC100041103 | 1.185737904 | 0.000299498 | 0.014892062 | 0.000299498 |
| ILMN_1213954 | SGK1 | 1.185317215 | 0.000299498 | 0.014892062 | 0.000299498 |
| ILMN_2632971 | B3GALT4 | 1.185249067 | 0.000299498 | 0.014892062 | 0.000299498 |
| ILMN_2860649 | GBP6 | 1.184770963 | 0.000299498 | 0.014892062 | 0.000299498 |
| ILMN_2722784 | CD3G | 1.184582149 | 0.000299498 | 0.014892062 | 0.000299498 |
| ILMN_2667829 | PRKCQ | 1.183849443 | 0.000299498 | 0.014892062 | 0.000299498 |
| ILMN_1235499 | PROS1 | 1.183674675 | 0.000299498 | 0.014892062 | 0.000299498 |
| ILMN_2694955 | IGFBP4 | 1.183278575 | 0.000299498 | 0.014892062 | 0.000299498 |
| ILMN_2491445 | LOC639001 | 1.183039504 | 0.000639896 | 0.021153497 | 0.000299498 |
| ILMN_2661185 | SCML4 | 1.18284704 | 0.000299498 | 0.014892062 | 0.000299498 |
| ILMN_2454823 | 6720418B01RIK | 1.182721445 | 0.000299498 | 0.014892062 | 0.000299498 |
| ILMN_2947526 | ECM1 | 1.181777956 | 0.000299498 | 0.014892062 | 0.000299498 |
| ILMN_2705407 | PABPC1 | 1.181328619 | 0.000942625 | 0.026071536 | 0.000639896 |
| ILMN_1227570 | LOC386545 | 1.180241912 | 0.000639896 | 0.021153497 | 0.000299498 |
| ILMN_2475156 | XIST | 1.179666178 | 0.000639896 | 0.021153497 | 0.000639896 |
| ILMN_1214650 | SCL0002368.1_75 | 1.179443236 | 0.000299498 | 0.014892062 | 0.000299498 |
| ILMN_2606825 | CMAH | 1.17926401 | 0.000299498 | 0.014892062 | 0.000299498 |
| ILMN_1224032 | SKAP1 | 1.178850818 | 0.000299498 | 0.014892062 | 0.000299498 |
| ILMN_2436183 | BCL11B | 1.178588337 | 0.000299498 | 0.014892062 | 0.000299498 |
| ILMN_2419490 | TNFRSF18 | 1.178043424 | 0.000299498 | 0.014892062 | 0.000299498 |
| ILMN_2432110 | LOC100046855 | 1.176564584 | 0.000299498 | 0.014892062 | 0.000299498 |
| ILMN_2443624 | BCL11B | 1.17644894 | 0.000299498 | 0.014892062 | 0.000299498 |
| ILMN_1217913 | D230007K08RIK | 1.176197032 | 0.000299498 | 0.014892062 | 0.000299498 |
| ILMN_1215649 | C230075M21RIK | 1.176099557 | 0.000299498 | 0.014892062 | 0.000299498 |
| ILMN_1217629 | ITGAE | 1.175144353 | 0.000299498 | 0.014892062 | 0.000299498 |
| ILMN_1215862 | CXCL9 | 1.174363745 | 0.001760487 | 0.038593802 | 0.001225688 |
| ILMN_2721399 | CTSW | 1.172718248 | 0.000299498 | 0.014892062 | 0.000299498 |
| ILMN_1248340 | LSG1 | 1.172636468 | 0.000299498 | 0.014892062 | 0.000299498 |
| ILMN_2618176 | SOCS3 | 1.171028633 | 0.000299498 | 0.014892062 | 0.000299498 |
| ILMN_1218717 | LOC385615 | 1.170940971 | 0.000299498 | 0.014892062 | 0.000299498 |
| ILMN_1247893 | CGEF2-PENDING | 1.169922593 | 0.000299498 | 0.014892062 | 0.000299498 |
| ILMN_1224473 | LOC380797 | 1.16992167 | 0.001225688 | 0.030541554 | 0.000942625 |
| ILMN_2419494 | TNFRSF18 | 1.169716279 | 0.000299498 | 0.014892062 | 0.000299498 |
| ILMN_2612125 | ARHGEF18 | 1.169661163 | 0.000299498 | 0.014892062 | 0.000299498 |
| ILMN_2606162 | PDLIM4 | 1.169633574 | 0.000639896 | 0.021153497 | 0.000299498 |
| ILMN_1217406 | 1110013L07RIK | 1.169153834 | 0.000299498 | 0.014892062 | 0.000299498 |
| ILMN_2568571 | C130079K02RIK | 1.168909165 | 0.000299498 | 0.014892062 | 0.000299498 |
| ILMN_2818964 | DUSP10 | 1.168854568 | 0.000299498 | 0.014892062 | 0.000299498 |
| ILMN_2431390 | TCRB-V8.2 | 1.167218786 | 0.000639896 | 0.021153497 | 0.000299498 |
| ILMN_2456911 | TRBV31_X03277_T_CELL_RECEPTOR_BETA_VARIABL | 1.166841194 | 0.000639896 | 0.021153497 | 0.000299498 |
| ILMN_2981169 | IFIT2 | 1.165766787 | 0.000299498 | 0.014892062 | 0.000299498 |
| ILMN_1249366 | LOC100046608 | 1.165718568 | 0.000299498 | 0.014892062 | 0.000299498 |
| ILMN_1246194 | LOC667370 | 1.165517165 | 0.000299498 | 0.014892062 | 0.000299498 |
| ILMN_1236507 | 5830496L11RIK | 1.164879502 | 0.000299498 | 0.014892062 | 0.000299498 |
| ILMN_1254692 | MS4A6B | 1.164318447 | 0.000299498 | 0.014892062 | 0.000299498 |
| ILMN_3156208 | GPR146 | 1.164011442 | 0.000299498 | 0.014892062 | 0.000299498 |
| ILMN_2734391 | RAMP1 | 1.16383138 | 0.000299498 | 0.014892062 | 0.000299498 |
| ILMN_1224091 | XIST | 1.16230787 | 0.000639896 | 0.021153497 | 0.000942625 |
| ILMN_2456216 | 5330403D14RIK | 1.161809103 | 0.000942625 | 0.026071536 | 0.000942625 |
| ILMN_2627441 | PELI1 | 1.161752176 | 0.000299498 | 0.014892062 | 0.000299498 |
| ILMN_2891245 | RHBDL2 | 1.161573842 | 0.002018395 | 0.042006944 | 0.002018395 |
| ILMN_1232804 | LOC276837 | 1.161420855 | 0.000299498 | 0.014892062 | 0.000299498 |
| ILMN_1232537 | ARL4C | 1.160980448 | 0.000299498 | 0.014892062 | 0.000299498 |
| ILMN_2444432 | TRBV1_AE000663_T_CELL_RECEPTOR_BETA_VARIA | 1.160799824 | 0.000299498 | 0.014892062 | 0.000299498 |
| ILMN_2756046 | FFAR2 | 1.160027707 | 0.000299498 | 0.014892062 | 0.000299498 |
| ILMN_1214163 | B230380D07RIK | 1.159421403 | 0.000299498 | 0.014892062 | 0.000299498 |
| ILMN_2613832 | MGST2 | 1.159082249 | 0.000299498 | 0.014892062 | 0.000299498 |
| ILMN_2506039 | A130090K04RIK | 1.158817015 | 0.000299498 | 0.014892062 | 0.000299498 |
| ILMN_1219179 | A830081L15RIK | 1.158794735 | 0.000299498 | 0.014892062 | 0.000299498 |
| ILMN_1248898 | A830092P18RIK | 1.158658732 | 0.000299498 | 0.014892062 | 0.000299498 |
| ILMN_2651575 | IFNGR1 | 1.158581155 | 0.000299498 | 0.014892062 | 0.000299498 |
| ILMN_2708580 | INADL | 1.158423303 | 0.000299498 | 0.014892062 | 0.000299498 |
| ILMN_1236256 | ARHGEF1 | 1.158058706 | 0.000299498 | 0.014892062 | 0.000639896 |
| ILMN_2525855 | FUS | 1.157139415 | 0.000299498 | 0.014892062 | 0.000299498 |
| ILMN_2526938 | LOC623121 | 1.157084959 | 0.000299498 | 0.014892062 | 0.000299498 |
| ILMN_1213664 | LOC665425 | 1.156699853 | 0.000942625 | 0.026071536 | 0.000639896 |
| ILMN_1255743 | IL6RA | 1.1564233 | 0.000299498 | 0.014892062 | 0.000299498 |
| ILMN_2646625 | JUN | 1.155896052 | 0.000299498 | 0.014892052 | 0.000299498 |
| ILMN_2843019 | KLHDC1 | 1.155520822 | 0.000299498 | 0.014892062 | 0.000299498 |
| ILMN_3047389 | GBP2 | 1.155409559 | 0.003970394 | 0.064249 | 0.004674836 |
| ILMN_1257639 | 0610009J05RIK | 1.155036694 | 0.000299498 | 0.014892062 | 0.000299498 |
| ILMN_1239770 | PELI1 | 1.154754524 | 0.000299498 | 0.014892062 | 0.000299498 |
| ILMN_2669841 | 6330581N18RIK | 1.153993411 | 0.000299498 | 0.014892062 | 0.000299498 |
| ILMN_2797061 | ACTN2 | 1.153924476 | 0.000299498 | 0.014892062 | 0.000299498 |
| ILMN_2784580 | CD3E | 1.153886402 | 0.000942625 | 0.026071536 | 0.000299498 |
| ILMN_3006219 | FYB | 1.153762655 | 0.000299498 | 0.014892062 | 0.000299498 |
| ILMN_2467190 | BAMBI-PS1 | 1.15354344 | 0.000299498 | 0.014892062 | 0.000299498 |
| ILMN_2536349 | LOC386513 | 1.153082558 | 0.000299498 | 0.014892062 | 0.000299498 |
| ILMN_2432550 | TRIB2 | 1.15299348 | 0.000299498 | 0.014892062 | 0.000299498 |
| ILMN_2844996 | ACTN1 | 1.152882536 | 0.000299498 | 0.014892062 | 0.000299498 |
| ILMN_1247281 | 5830468F06RIK | 1.15264746 | 0.000299498 | 0.014892062 | 0.000299498 |
| ILMN_1227814 | SRR | 1.1524679 | 0.004441009 | 0.0691859 | 0.003733327 |
| ILMN_1249021 | BCL2 | 1.152415672 | 0.000299498 | 0.014892062 | 0.000299498 |
| ILMN_2427592 | LOC546630 | 1.152263699 | 0.000299498 | 0.014892062 | 0.000299498 |
| ILMN_1259075 | NME7 | 1.152030836 | 0.000299498 | 0.014892062 | 0.000299498 |
| ILMN_1343049 | control_ILMN_1343049 | 1.151797691 | 0.000639896 | 0.021153497 | 0.000639896 |
| ILMN_3026397 | CHKA | 1.1512093 | 0.003733327 | 0.06184411 | 0.003970394 |
| ILMN_2631610 | TMEM71 | 1.15055696 | 0.000299498 | 0.014892062 | 0.000299498 |
| ILMN_1227434 | ITGB7 | 1.150210635 | 0.000639896 | 0.021153497 | 0.000299498 |
| ILMN_1232600 | RNF125 | 1.150131069 | 0.000299498 | 0.014892062 | 0.000299498 |
| ILMN_2656854 | MYO6 | 1.149171076 | 0.000299498 | 0.014892062 | 0.000299498 |
| ILMN_1258652 | RBBP2 | 1.148685264 | 0.000299498 | 0.014892062 | 0.000299498 |
| ILMN_1245754 | CD84 | 1.148512188 | 0.000299498 | 0.014892062 | 0.000299498 |
| ILMN_2780247 | LTA | 1.147812483 | 0.001496892 | 0.034781534 | 0.000942625 |
| ILMN_2663930 | SLFN1 | 1.147768768 | 0.000299498 | 0.014892062 | 0.000299498 |
| ILMN_3023230 | JMJD3 | 1.147753644 | 0.000299498 | 0.014892062 | 0.000299498 |
| ILMN_2764727 | ACTN2 | 1.146764113 | 0.000299498 | 0.014892062 | 0.000299498 |
| ILMN_2539295 | LOC621968 | 1.14667536 | 0.001496892 | 0.034781534 | 0.000639896 |
| ILMN_2448404 | SCL0002975.1_346 | 1.146129707 | 0.001760487 | 0.038593802 | 0.002018395 |
| ILMN_1241915 | NOTCH1 | 1.145832115 | 0.000299498 | 0.014892062 | 0.000299498 |
| ILMN_2605819 | EGLN3 | 1.145595193 | 0.000639896 | 0.021153497 | 0.000639896 |
| ILMN_1253699 | 2900045G02RIK | 1.145548347 | 0.000299498 | 0.014892062 | 0.000299498 |
| ILMN_1244853 | LOC100044948 | 1.14491469 | 0.000299498 | 0.014892062 | 0.000299498 |
| ILMN_2589871 | CD28 | 1.144678691 | 0.000639896 | 0.021153497 | 0.000299498 |
| ILMN_2997406 | ARL4C | 1.144292866 | 0.000942625 | 0.026071536 | 0.000639896 |
| ILMN_2860645 | GBP6 | 1.143811908 | 0.000639896 | 0.021153497 | 0.000639896 |
| ILMN_1249547 | ZFP292 | 1.143548873 | 0.000299498 | 0.014892062 | 0.000299498 |
| ILMN_1236539 | PFKL | 1.142647393 | 0.000299498 | 0.014892062 | 0.000299498 |
| ILMN_2425990 | 4933421G18RIK | 1.142576402 | 0.000299498 | 0.014892062 | 0.000299498 |
| ILMN_2553041 | A130093I21RIK | 1.142422921 | 0.001760487 | 0.038593802 | 0.00227139 |
| ILMN_1224945 | E130113K22RIK | 1.141736556 | 0.000299498 | 0.014892062 | 0.000299498 |
| ILMN_1218525 | IL18R1 | 1.141400055 | 0.000299498 | 0.014892062 | 0.000299498 |
| ILMN_1243621 | A130026C10RIK | 1.141355268 | 0.000299498 | 0.014892062 | 0.000299498 |
| ILMN_1228608 | LOC637353 | 1.141241268 | 0.003733327 | 0.06184411 | 0.003494726 |
| ILMN_1229746 | ECM1 | 1.141034556 | 0.000299498 | 0.014892062 | 0.000299498 |
| ILMN_2915951 | D13ERTD608E | 1.140936106 | 0.000299498 | 0.014892062 | 0.000299498 |
| ILMN_1249864 | A630077B13RIK | 1.140911297 | 0.000942625 | 0.026071536 | 0.000299498 |
| ILMN_2995934 | LOC547323 | 1.140861547 | 0.000299498 | 0.014892062 | 0.000299498 |
| ILMN_2456391 | TCRB-J | 1.140797339 | 0.001760487 | 0.038593802 | 0.000299498 |
| ILMN_1239469 | LEF1 | 1.140750182 | 0.000299498 | 0.014892062 | 0.000299498 |
| ILMN_1259463 | ANKZF1 | 1.140723241 | 0.000299498 | 0.014892062 | 0.000299498 |
| ILMN_3142803 | CXCL10 | 1.140155785 | 0.003253905 | 0.056782136 | 0.003012339 |
| ILMN_2594768 | THA1 | 1.139893861 | 0.001496892 | 0.034781534 | 0.000942625 |
| ILMN_2677207 | LOC100047788 | 1.139882555 | 0.005600396 | 0.080174568 | 0.004907577 |
| ILMN_1226048 | AI504432 | 1.13986418 | 0.000299498 | 0.014892062 | 0.000299498 |
| ILMN_1241598 | LY116 | 1.139617809 | 0.000299498 | 0.014892062 | 0.000299498 |
| ILMN_1258272 | LOC382646 | 1.139404236 | 0.001496892 | 0.034781534 | 0.001225688 |
| ILMN_1238847 | CD3E | 1.139135835 | 0.000639896 | 0.021153497 | 0.000299498 |
| ILMN_1254218 | NISCH | 1.138664045 | 0.000299498 | 0.014892062 | 0.000299498 |
| ILMN_2879858 | PRKX | 1.138650161 | 0.000299498 | 0.014892062 | 0.000299498 |
| ILMN_1231490 | 2410006H16RIK | 1.138597457 | 0.000299498 | 0.014892062 | 0.000299498 |
| ILMN_1232707 | SAMHD1 | 1.138552379 | 0.000299498 | 0.014892062 | 0.000299498 |
| ILMN_1240592 | ALS2CL | 1.138470539 | 0.01404445 | 0.135546677 | 0.011420742 |
| ILMN_3142602 | B3GALT4 | 1.138320625 | 0.000299498 | 0.014892062 | 0.000299498 |
| ILMN_3031781 | ARID5B | 1.138169635 | 0.000639896 | 0.021153497 | 0.000942625 |
| ILMN_2568028 | IL2RG | 1.138046812 | 0.000639896 | 0.021153497 | 0.000299498 |
| ILMN_1239776 | LOC100040243 | 1.1379032 | 0.000942625 | 0.026071536 | 0.000639896 |
| ILMN_1253691 | 8430201A12RIK | 1.137684368 | 0.000299498 | 0.014892062 | 0.000299498 |
| ILMN_1239238 | PRKCH | 1.137598283 | 0.000299498 | 0.014892062 | 0.000299498 |
| ILMN_2663230 | SLCO3A1 | 1.137294887 | 0.000942625 | 0.026071536 | 0.000639896 |
| ILMN_1255869 | CATNB | 1.137232235 | 0.000299498 | 0.014892062 | 0.000299498 |
| ILMN_2847773 | OTTMUSG00000005523 | 1.137208625 | 0.000299498 | 0.014892062 | 0.000299498 |
| ILMN_2945940 | RAPGEF4 | 1.136953118 | 0.000299498 | 0.014892062 | 0.000299498 |
| ILMN_2426853 | UBD | 1.13683555 | 0.001496892 | 0.034781534 | 0.001496892 |
| ILMN_2639972 | PPP1R3B | 1.136776701 | 0.000299498 | 0.014892062 | 0.000299498 |
| ILMN_2693461 | SSBP2 | 1.136455877 | 0.000299498 | 0.014892062 | 0.000299498 |
| ILMN_1258988 | IGFBP4 | 1.136364638 | 0.006058437 | 0.083694614 | 0.003970394 |
| ILMN_2509988 | LBR | 1.136214053 | 0.000299498 | 0.014892062 | 0.000299498 |
| ILMN_1230458 | IFIT3 | 1.136194338 | 0.002018395 | 0.042006944 | 0.001496892 |
| ILMN_2727235 | ANKRD11 | 1.136184371 | 0.002018395 | 0.042006944 | 0.001225688 |
| ILMN_2637714 | RASA3 | 1.135912258 | 0.000299498 | 0.014892062 | 0.000299498 |
| ILMN_2595918 | GIMAP7 | 1.135856716 | 0.000639896 | 0.021153497 | 0.000639896 |
| ILMN_1231573 | SERPINB1A | 1.135730392 | 0.000299498 | 0.014892062 | 0.000299498 |
| ILMN_2459155 | ZFP238 | 1.135647435 | 0.000299498 | 0.014892062 | 0.000299498 |
| ILMN_1243910 | ZFP292 | 1.13546193 | 0.000639896 | 0.021153497 | 0.000299498 |
| ILMN_1222471 | GMFG | 1.13543581 | 0.000299498 | 0.014892062 | 0.000299498 |
| ILMN_2469190 | B230345P09RIK | 1.135305336 | 0.000639896 | 0.021153497 | 0.000942625 |
| ILMN_2696492 | PHF11 | 1.135183534 | 0.000942625 | 0.026071536 | 0.000639896 |
| ILMN_2996904 | OBFC2A | 1.135148949 | 0.000299498 | 0.014892062 | 0.000299498 |
| ILMN_1233449 | LOC100044430 | 1.13513543 | 0.000299498 | 0.014892062 | 0.000299498 |
| ILMN_1251193 | A630084D02RIK | 1.134829034 | 0.000299498 | 0.014892062 | 0.000299498 |
| ILMN_2758878 | TMEM66 | 1.13478407 | 0.002768024 | 0.051337774 | 0.001760487 |
| ILMN_3160137 | ALDOC | 1.134554634 | 0.007644699 | 0.096726012 | 0.005830016 |
| ILMN_1218051 | IQGAP2 | 1.134418217 | 0.001496892 | 0.034781534 | 0.001225688 |
| ILMN_1230726 | 5430406J06RIK | 1.13436247 | 0.001225688 | 0.030541554 | 0.000942625 |
| ILMN_2879614 | ZBP1 | 1.13418419 | 0.000639896 | 0.021153497 | 0.000639896 |
| ILMN_2534207 | LOC380706 | 1.133996759 | 0.000299498 | 0.014892062 | 0.000299498 |
| ILMN_2659739 | IL7R | 1.133870404 | 0.000299498 | 0.014892062 | 0.000299498 |
| ILMN_2958159 | ENO1 | 1.13384821 | 0.000299498 | 0.014892062 | 0.000299498 |
| ILMN_1257704 | 6030458P06RIK | 1.133768716 | 0.000639896 | 0.021153497 | 0.000639896 |
| ILMN_2477213 | LOC100045240 | 1.133690768 | 0.000299498 | 0.014892062 | 0.000299498 |
| ILMN_2513870 | ZAP70 | 1.133508982 | 0.000299498 | 0.014892062 | 0.000299498 |
| ILMN_1343061 | control_ILMN_1343061 | 1.133413667 | 0.058960045 | 0.296529118 | 0.053139043 |
| ILMN_1258340 | TCF12 | 1.133249325 | 0.000299498 | 0.014892062 | 0.000299498 |
| ILMN_2777019 | SPO11 | 1.133035275 | 0.000639896 | 0.021153497 | 0.000639896 |
| ILMN_1255907 | ARP2 | 1.132980051 | 0.000299498 | 0.014892062 | 0.000299498 |
| ILMN_2449620 | 5830427D02RIK | 1.132961187 | 0.000299498 | 0.014892062 | 0.000299498 |
| ILMN_2477946 | SCL0001464.1_61 | 1.132866527 | 0.000299498 | 0.014892062 | 0.000299498 |
| ILMN_2627179 | ELL3 | 1.132790682 | 0.000299498 | 0.014892062 | 0.000299498 |
| ILMN_1214318 | RASGRP1 | 1.132335612 | 0.000639896 | 0.021153497 | 0.000299498 |
| ILMN_1218206 | KLF13 | 1.132335512 | 0.002018395 | 0.042006944 | 0.001496892 |
| ILMN_1233501 | C530027B15RIK | 1.132246044 | 0.001225688 | 0.030541554 | 0.001225688 |
| ILMN_1241522 | 4921505C17RIK | 1.13221457 | 0.000299498 | 0.014892062 | 0.000299498 |
| ILMN_1226239 | INPP4B | 1.132137878 | 0.001760487 | 0.038593802 | 0.001760487 |
| ILMN_2613422 | ITK | 1.131935528 | 0.000639896 | 0.021153497 | 0.000639896 |
| ILMN_2593554 | IGTP | 1.131930192 | 0.000299498 | 0.014892062 | 0.000299498 |
| ILMN_1229804 | LTA | 1.131546852 | 0.005830016 | 0.082146349 | 0.002520973 |
| ILMN_2607160 | PDM2 | 1.130377924 | 0.000639896 | 0.021153497 | 0.000299498 |
| ILMN_3072437 | 633O500DO4RIK | 1.130133265 | 0.003012339 | 0.054401128 | 0.002520973 |
| ILMN_1251170 | AFF4 | 1.130045502 | 0.000299498 | 0.014892062 | 0.000299498 |
| ILMN_1246582 | LOC436541 | 1.129946125 | 0.000639896 | 0.021153497 | 0.000639896 |
| ILMN_1244891 | CST7 | 1.1298809 | 0.000299498 | 0.014892062 | 0.000299498 |
| ILMN_2697693 | GRHPR | 1.129040446 | 0.000639896 | 0.021153497 | 0.000639896 |
| ILMN_1233402 | LOC100045981 | 1.129004666 | 0.000299498 | 0.014892062 | 0.000299498 |
| ILMN_1258864 | BC106179 | 1.128947865 | 0.000299498 | 0.014892062 | 0.000639896 |
| ILMN_1234796 | HSD17B12 | 1.128868369 | 0.000299498 | 0.014892062 | 0.000299498 |
| ILMN_1236595 | SLA2 | 1.128559293 | 0.001760487 | 0.038593802 | 0.001225688 |
| ILMN_1241952 | 6230415M23RIK | 1.12852499 | 0.000299498 | 0.014892062 | 0.000639896 |
| ILMN_2657980 | FAAH | 1.128386309 | 0.000299498 | 0.014892062 | 0.000299498 |
| ILMN_2745614 | FAM134B | 1.127986989 | 0.000299498 | 0.014892062 | 0.000299498 |
| ILMN_1240415 | LOC234987 | 1.127947991 | 0.000299498 | 0.014892062 | 0.000299498 |
| ILMN_2552490 | 6720463L11RIK | 1.127757726 | 0.000639896 | 0.021153497 | 0.000299498 |
| ILMN_2729447 | 9030612M13RIK | 1.12772184 | 0.000299498 | 0.014892062 | 0.000299498 |
| ILMN_1233293 | GBP1 | 1.127491461 | 0.000299498 | 0.014892062 | 0.000299498 |
| ILMN_1236105 | FYB | 1.127293421 | 0.001760487 | 0.038593802 | 0.002018395 |
| ILMN_3009860 | SELL | 1.126985915 | 0.000639896 | 0.021153497 | 0.000299498 |
| ILMN_1217929 | LOC667005 | 1.126757091 | 0.000942625 | 0.026071536 | 0.000942625 |
| ILMN_1219896 | LOC623121 | 1.126643386 | 0.000299498 | 0.014892062 | 0.000639896 |
| ILMN_2808485 | GBP10 | 1.126535797 | 0.000942625 | 0.026071536 | 0.000942625 |
| ILMN_2628271 | LOC100045967 | 1.126530154 | 0.001225688 | 0.030541554 | 0.000639896 |
| ILMN_2959272 | RNU6 | 1.126488141 | 0.000299498 | 0.014892062 | 0.000299498 |
| ILMN_1240857 | COX7A1 | 1.126488113 | 0.000299498 | 0.014892062 | 0.000299498 |
| ILMN_1248837 | TBXA2R | 1.126308353 | 0.000639896 | 0.021153497 | 0.000639896 |
| ILMN_1213986 | LOC380617 | 1.126269609 | 0.000299498 | 0.014892062 | 0.000299498 |
| ILMN_2565089 | A830083H19RIK | 1.126188031 | 0.000299498 | 0.014892062 | 0.000299498 |
| ILMN_2756628 | CMC1 | 1.125722229 | 0.000299498 | 0.014892062 | 0.000299498 |
| ILMN_2689307 | SPNB2 | 1.125305551 | 0.000299498 | 0.014892062 | 0.000299498 |
| ILMN_2551741 | 0610010I05RIK | 1.124918642 | 0.000639896 | 0.021153497 | 0.000639896 |
| ILMN_1343062 | control_ILMN_1343062 | 1.124836817 | 0.065181711 | 0.311898271 | 0.059582948 |
| ILMN_2749364 | LOC382646 | 1.124580104 | 0.002768024 | 0.051337774 | 0.001760487 |
| ILMN_2784272 | IFNGR2 | 1.124504822 | 0.002018395 | 0.042006944 | 0.001760487 |
| ILMN_1225348 | FAM102A | 1.12448091 | 0.000299498 | 0.014892062 | 0.000299498 |
| ILMN_2630739 | PHF20L1 | 1.124360297 | 0.000942625 | 0.026071536 | 0.001496892 |
| ILMN_2418366 | 6720427H10RIK | 1.124259069 | 0.000299498 | 0.014892062 | 0.000299498 |
| ILMN_2846731 | 1110059E24RIK | 1.124049613 | 0.000299498 | 0.014892062 | 0.000299498 |
| ILMN_2594855 | PDPK1 | 1.124023837 | 0.000299498 | 0.014892062 | 0.000299498 |
| ILMN_1246543 | SIAT7C | 1.124022495 | 0.000299498 | 0.014892062 | 0.000299498 |
| ILMN_2614884 | PJA1 | 1.123647984 | 0.000299498 | 0.014892062 | 0.000299498 |
| ILMN_1250418 | NPC1 | 1.123625032 | 0.000299498 | 0.014892062 | 0.000299498 |
| ILMN_2706514 | LOC100046608 | 1.123601186 | 0.000942625 | 0.026071536 | 0.000639896 |
| ILMN_1241924 | ARL6IP5 | 1.123582409 | 0.000299498 | 0.014892062 | 0.000299498 |
| ILMN_2750265 | ELMO1 | 1.123418741 | 0.001225688 | 0.030541554 | 0.001225688 |
| ILMN_2712895 | 4631423B10RIK | 1.123402203 | 0.000299498 | 0.014892062 | 0.000299498 |
| ILMN_2719139 | AB124611 | 1.122548641 | 0.001225688 | 0.030541554 | 0.000639896 |
| ILMN_2692986 | 2810410P22RIK | 1.122423336 | 0.001496892 | 0.034781534 | 0.001225688 |
| ILMN_1228333 | PRF1 | 1.122189532 | 0.002768024 | 0.051337774 | 0.001225688 |
| ILMN_2940446 | DGKA | 1.122160698 | 0.001760487 | 0.038593802 | 0.001496892 |
| ILMN_3034381 | NUP88 | 1.121988063 | 0.000942625 | 0.026071536 | 0.000942625 |
| ILMN_2668387 | CGGBP1 | 1.121946931 | 0.000299498 | 0.014892062 | 0.000299498 |
| ILMN_2592953 | SCG5 | 1.121946089 | 0.000299498 | 0.014892062 | 0.000299498 |
| ILMN_1228867 | A130082M07RIK | 1.121929865 | 0.000942625 | 0.026071536 | 0.000299498 |
| ILMN_2785454 | HIST2H2AB | 1.121848738 | 0.008987421 | 0.105475934 | 0.009654071 |
| ILMN_2773900 | GLIPR2 | 1.1217158 | 0.000639896 | 0.021153497 | 0.000639896 |
| ILMN_2647102 | ACTL6A | 1.121390591 | 0.000299498 | 0.014892062 | 0.000299498 |
| ILMN_1256574 | NCOA2 | 1.121377157 | 0.000942625 | 0.026071536 | 0.000942625 |
| ILMN_1243902 | A530024C08RIK | 1.12112103 | 0.002018395 | 0.042006944 | 0.001760487 |
| ILMN_1343048 | control_ILMN_1343048 | 1.120964441 | 0.000639896 | 0.021153497 | 0.000639896 |
| ILMN_2878979 | TNFAIP8L2 | 1.120676477 | 0.000299498 | 0.014892062 | 0.000299498 |
| ILMN_2887619 | RPS25 | 1.120128473 | 0.032410957 | 0.214642335 | 0.037882791 |
| ILMN_2678724 | DUSP10 | 1.119986223 | 0.000639896 | 0.021153497 | 0.000299498 |
| ILMN_1244135 | ARPC5 | 1.119864481 | 0.003012339 | 0.054401128 | 0.003733327 |
| ILMN_2651297 | 4632428N05RIK | 1.119607561 | 0.000299498 | 0.014892062 | 0.000299498 |
| ILMN_2822842 | 1600014C10RIK | 1.119392625 | 0.000299498 | 0.014892062 | 0.000299498 |
| ILMN_2665545 | RIN3 | 1.119281709 | 0.000299498 | 0.014892062 | 0.000299498 |
| ILMN_2703321 | ANKZF1 | 1.119253 | 0.000299498 | 0.014892062 | 0.000299498 |
| ILMN_1259917 | D330001F19RIK | 1.119179745 | 0.000299498 | 0.014892062 | 0.000299498 |
| ILMN_1237871 | AMPD1 | 1.119072973 | 0.000299498 | 0.014892062 | 0.000299498 |
| ILMN_1256950 | NEURL2 | 1.11903277 | 0.000639896 | 0.021153497 | 0.000639896 |
| ILMN_1228020 | 1500010G04RIK | 1.118985348 | 0.001225688 | 0.030541554 | 0.000942625 |
| ILMN_1227573 | IRF7 | 1.118870281 | 0.003970394 | 0.064249 | 0.003970394 |
| ILMN_2726315 | SDF4 | 1.118652369 | 0.001496892 | 0.034781534 | 0.000639896 |
| ILMN_1227907 | GMFG | 1.11859249 | 0.000299498 | 0.014892062 | 0.000299498 |
| ILMN_2881296 | TMEM66 | 1.118516263 | 0.000299498 | 0.014892062 | 0.000299498 |
| ILMN_3043245 | JMJD1A | 1.118512017 | 0.000299498 | 0.014892062 | 0.000299498 |
| ILMN_2580159 | C230070D10RIK | 1.118494179 | 0.000942625 | 0.026071536 | 0.000942625 |
| ILMN_1252472 | ITK | 1.118457931 | 0.00227139 | 0.045458063 | 0.002768024 |
| ILMN_2828916 | FRMD6 | 1.11830741 | 0.000639896 | 0.021153497 | 0.000639896 |
| ILMN_1257771 | LOC638301 | 1.118249781 | 0.001496892 | 0.034781534 | 0.000942625 |
| ILMN_3002095 | IL27RA | 1.118221343 | 0.000639896 | 0.021153497 | 0.000639896 |
| ILMN_2658961 | DGKA | 1.118166028 | 0.001760487 | 0.038593802 | 0.001760487 |
| ILMN_2038771 | control_ILMN_2038771 | 1.118161322 | 0.060205719 | 0.300139071 | 0.054803588 |
| ILMN_2981801 | HIST1H2AG | 1.118065046 | 0.000942625 | 0.026071536 | 0.000639896 |
| ILMN_1249710 | E030038D23RIK | 1.117909411 | 0.000299498 | 0.014892062 | 0.000299498 |
| ILMN_2943057 | ARL5C | 1.117880079 | 0.001760487 | 0.038593802 | 0.000942625 |
| ILMN_1235856 | 8030474H12RIK | 1.117810336 | 0.000299498 | 0.014892062 | 0.000299498 |
| ILMN_1247942 | MGEA5 | 1.117806646 | 0.000639896 | 0.021153497 | 0.000639896 |
| ILMN_1258509 | SIT1 | 1.117432738 | 0.000639896 | 0.021153497 | 0.000299498 |
| ILMN_2544056 | HBB-B1 | 1.117213432 | 0.039141764 | 0.237049914 | 0.035991476 |
| ILMN_2729953 | PPM1B | 1.117036433 | 0.005370369 | 0.078183323 | 0.005370369 |
| ILMN_1219602 | LOC100048770 | 1.116440805 | 0.003970394 | 0.064249 | 0.003733327 |
| ILMN_2681670 | VPS37B | 1.116406759 | 0.000299498 | 0.014892062 | 0.000299498 |
| ILMN_1234955 | 6430573H23RIK | 1.116406299 | 0.001760487 | 0.038593802 | 0.002018395 |
| ILMN_2582659 | E030040J04RIK | 1.116391042 | 0.000299498 | 0.014892062 | 0.000299498 |
| ILMN_1252514 | C920016N10RIK | 1.116354745 | 0.001760487 | 0.038593802 | 0.001225688 |
| ILMN_2617433 | ACTB | 1.11629809 | 0.003970394 | 0.064249 | 0.003253905 |
| ILMN_2738972 | CNOT4 | 1.116182237 | 0.000299498 | 0.014892062 | 0.000299498 |
| ILMN_2553280 | 4732470M22RIK | 1.1161327 | 0.000299498 | 0.014892062 | 0.000299498 |
| ILMN_1238725 | C130086J11RIK | 1.116108151 | 0.013826671 | 0.134561326 | 0.015131078 |
| ILMN_2998813 | SEMA4F | 1.115968974 | 0.000299498 | 0.014892062 | 0.000299498 |
| ILMN_1236445 | 9130227N12RIK | 1.115922245 | 0.002520973 | 0.048392748 | 0.001760487 |
| ILMN_1220893 | ZFP281 | 1.115869505 | 0.000299498 | 0.014892062 | 0.000299498 |
| ILMN_2417398 | IGHV10S1_AF064442_IG_HEAVY_VARIABLE_10S1_1 | 1.115826459 | 0.004907577 | 0.073992984 | 0.005600396 |
| ILMN_1238893 | LIME1 | 1.115781178 | 0.001760487 | 0.038593802 | 0.000639896 |
| ILMN_2706550 | SUSD3 | 1.115744092 | 0.001225688 | 0.030541554 | 0.000942625 |
| ILMN_1242235 | TOP2A | 1.115488708 | 0.000639896 | 0.021153497 | 0.000299498 |
| ILMN_2638923 | RN18S | 1.115471516 | 0.003012339 | 0.054401128 | 0.005830016 |
| ILMN_1232894 | ZNRF1 | 1.115445926 | 0.000299498 | 0.014892062 | 0.000299498 |
| ILMN_1218799 | EMB | 1.115430499 | 0.000299498 | 0.014892062 | 0.000299498 |
| ILMN_1257209 | WIPF1 | 1.115376002 | 0.000299498 | 0.014892062 | 0.000299498 |
| ILMN_2711705 | DUSP11 | 1.115060696 | 0.000299498 | 0.014892062 | 0.000299498 |
| ILMN_1236202 | C430015M08RIK | 1.114991211 | 0.000639896 | 0.021153497 | 0.000942625 |
| ILMN_1233474 | IL2RA | 1.114952254 | 0.00809366 | 0.100146322 | 0.005370369 |
| ILMN_2762863 | AI606I8I | 1.114892504 | 0.000639896 | 0.021153497 | 0.000299498 |
| ILMN_2644350 | THY1 | 1.114766581 | 0.000942625 | 0.026071536 | 0.000639896 |
| ILMN_1255860 | KLRD1 | 1.114664485 | 0.000299498 | 0.014892062 | 0.000299498 |
| ILMN_2636403 | AXUD1 | 1.11465607 | 0.000299498 | 0.014892062 | 0.000299498 |
| ILMN_2918002 | GBP3 | 1.114435116 | 0.000942625 | 0.026071536 | 0.000299498 |
| ILMN_2649068 | IRF1 | 1.114384547 | 0.007194081 | 0.092947182 | 0.007194081 |
| ILMN_1215939 | 1_M15618_T_CELL_RECEPTOR_BETA_VARIABLE_13-1_213 | 1.114252325 | 0.000639896 | 0.021153497 | 0.000639896 |
| ILMN_2520264 | 2010016I18RIK | 1.114246403 | 0.001225688 | 0.030541554 | 0.001496892 |
| ILMN_1220889 | NBR1 | 1.114192214 | 0.000299498 | 0.014892062 | 0.000299498 |
| ILMN_2706803 | CRLF3 | 1.114104774 | 0.000299498 | 0.014892062 | 0.000299498 |
| ILMN_2751603 | PHXR4 | 1.11385586 | 0.008764316 | 0.104351101 | 0.01317262 |
| ILMN_1240030 | NFATC3 | 1.113638258 | 0.000299498 | 0.014892062 | 0.000299498 |
| ILMN_1242390 | ACTN4 | 1.113610426 | 0.006741497 | 0.089431865 | 0.005370369 |
| ILMN_2510694 | NME7 | 1.113521644 | 0.002018395 | 0.042006944 | 0.001760487 |
| ILMN_1258466 | LOC380623 | 1.113479163 | 0.001496892 | 0.034781534 | 0.001225688 |
| ILMN_1252089 | NFATC3 | 1.113257321 | 0.000942625 | 0.026071536 | 0.000639896 |
| ILMN_2522571 | SETD7 | 1.113080478 | 0.000299498 | 0.014892062 | 0.000299498 |
| ILMN_2593002 | ST6GAL2 | 1.113003988 | 0.000299498 | 0.014892062 | 0.000299498 |
| ILMN_1248379 | EG434077 | 1.112625375 | 0.000299498 | 0.014892062 | 0.000299498 |
| ILMN_1223119 | BCL11B | 1.112508226 | 0.002018395 | 0.042006944 | 0.001225688 |
| ILMN_3059798 | FOXK1 | 1.112313504 | 0.001225688 | 0.030541554 | 0.000942625 |
| ILMN_2534794 | LOC236170 | 1.112310228 | 0.005139198 | 0.076197614 | 0.004206115 |
| ILMN_2618549 | RAB7 | 1.112121637 | 0.000639896 | 0.021153497 | 0.001225688 |
| ILMN_2468405 | 5630401D06RIK | 1.111986642 | 0.000299498 | 0.014892062 | 0.000299498 |
| ILMN_1258587 | LOC100044376 | 1.111863958 | 0.000299498 | 0.014892062 | 0.000299498 |
| ILMN_1218732 | 2810426P10RIK | 1.11161873 | 0.000299498 | 0.014892062 | 0.000299498 |
| ILMN_1237980 | 0610010D24RIK | 1.111616983 | 0.000639896 | 0.021153497 | 0.000299498 |
| ILMN_1214874 | LOC386068 | 1.111606821 | 0.000942625 | 0.026071536 | 0.000299498 |
| ILMN_1220418 | HCST | 1.111606518 | 0.000299498 | 0.014892062 | 0.000299498 |
| ILMN_2550291 | 2310032M22RIK | 1.111606315 | 0.000639896 | 0.021153497 | 0.000639896 |
| ILMN_1234202 | PHIP | 1.111345184 | 0.000299498 | 0.014892062 | 0.000942625 |
| ILMN_2426949 | TNFRSF14 | 1.111293325 | 0.000639896 | 0.021153497 | 0.000639896 |
| ILMN_2497395 | 9130004J05RIK | 1.111130133 | 0.001225688 | 0.030541554 | 0.001225688 |
| ILMN_2715730 | SPN | 1.111033738 | 0.000299498 | 0.014892062 | 0.000639896 |
| ILMN_1217606 | 1500005K14RIK | 1.110936144 | 0.001225688 | 0.030541554 | 0.001496892 |
| ILMN_1216313 | STIM1 | 1.110931979 | 0.001225688 | 0.030541554 | 0.00227139 |
| ILMN_1252761 | DRG1 | 1.110887212 | 0.001225688 | 0.030541554 | 0.000639896 |
| ILMN_2710312 | LOC100046650 | 1.110811588 | 0.000299498 | 0.014892062 | 0.000299498 |
| ILMN_1232668 | MAD | 1.110784132 | 0.000299498 | 0.014892062 | 0.000299498 |
| ILMN_2593774 | 1190002H23RIK | 1.110704 | 0.000299498 | 0.014892062 | 0.000299498 |
| ILMN_2559724 | A130014O09RIK | 1.110598097 | 0.011420742 | 0.120583495 | 0.008764316 |
| ILMN_3002505 | ABHD8 | 1.110530454 | 0.000942625 | 0.026071536 | 0.000942625 |
| ILMN_2441724 | 9430014F16RIK | 1.110454831 | 0.000639896 | 0.021153497 | 0.000639896 |
| ILMN_1229005 | TRAT1 | 1.110421282 | 0.000639896 | 0.021153497 | 0.000639896 |
| ILMN_1229518 | B230340J04RIK | 1.11037499 | 0.000299498 | 0.014892062 | 0.000299498 |
| ILMN_2576568 | D130062J21RIK | 1.110225832 | 0.001760487 | 0.038593802 | 0.002768024 |
| ILMN_2491182 | A130010C12RIK | 1.110126472 | 0.014262117 | 0.136875566 | 0.017939699 |
| ILMN_2484278 | 9930031P18RIK | 1.110094091 | 0.002520973 | 0.048392748 | 0.003012339 |
| ILMN_2827780 | D14ERTD668E | 1.11008286 | 0.000639896 | 0.021153497 | 0.000299498 |
| ILMN_3112873 | TXNIP | 1.110031984 | 0.000942625 | 0.026071536 | 0.000639896 |
| ILMN_3117602 | CD6 | 1.109929013 | 0.000299498 | 0.014892062 | 0.000299498 |
| ILMN_1233424 | LBCL1 | 1.109898196 | 0.000299498 | 0.014892062 | 0.000299498 |
| ILMN_2681601 | SLC44A2 | 1.109888463 | 0.010096998 | 0.112851217 | 0.008541654 |
| ILMN_2762956 | SPCS2 | 1.109838126 | 0.000299498 | 0.014892062 | 0.000299498 |
| ILMN_2632230 | D2ERTD391E | 1.109720922 | 0.000299498 | 0.014892062 | 0.000299498 |
| ILMN_3052501 | CD27 | 1.109550882 | 0.000942625 | 0.026071536 | 0.000639896 |
| ILMN_2615096 | DPP4 | 1.109531905 | 0.000299498 | 0.014892062 | 0.000299498 |
| ILMN_2629112 | ASAH3L | 1.109478785 | 0.000299498 | 0.014892062 | 0.000299498 |
| ILMN_1254916 | LOC386508 | 1.109314765 | 0.000942625 | 0.026071536 | 0.000639896 |
| ILMN_2686115 | RAPGEF6 | 1.109219239 | 0.006058437 | 0.083694614 | 0.004441009 |
| ILMN_3153010 | SLITRK5 | 1.109095163 | 0.000299498 | 0.014892062 | 0.000299498 |
| ILMN_2571616 | C430002D13RIK | 1.109088506 | 0.001225688 | 0.030541554 | 0.002018395 |
| ILMN_1231785 | HDAC2 | 1.10904434 | 0.000639896 | 0.021153497 | 0.000299498 |
| ILMN_1221831 | TCFAP2E | 1.10874714 | 0.001760487 | 0.038593802 | 0.001760487 |
| ILMN_2646456 | RNPC2 | 1.108556752 | 0.003970394 | 0.064249 | 0.003494726 |
| ILMN_2504686 | MT-ATP6 | 1.108358727 | 0.008764316 | 0.104351101 | 0.008987421 |
| ILMN_2712867 | TIMP2 | 1.108193843 | 0.000299498 | 0.014892062 | 0.000299498 |
| ILMN_1234006 | LOC277136 | 1.10816163 | 0.003733327 | 0.06184411 | 0.002018395 |
| ILMN_2965669 | XLR4A | 1.108141291 | 0.000299498 | 0.014892062 | 0.000299498 |
| ILMN_2447538 | 2210018M11RIK | 1.10801562 | 0.000639896 | 0.021153497 | 0.000639896 |
| ILMN_1222917 | PTPN1 | 1.107980121 | 0.003253905 | 0.056782136 | 0.003733327 |
| ILMN_1255086 | 2810454F19RIK | 1.107686745 | 0.006741497 | 0.089431865 | 0.006514138 |
| ILMN_1233554 | PBRM1 | 1.107679536 | 0.001496892 | 0.034781534 | 0.000942625 |
| ILMN_1234072 | PDLIM1 | 1.107488297 | 0.000299498 | 0.014892062 | 0.000299498 |
| ILMN_1216561 | CAMK2D | 1.107443275 | 0.001225688 | 0.030541554 | 0.000942625 |
| ILMN_2590638 | BCL2L11 | 1.107365706 | 0.001225688 | 0.030541554 | 0.001225688 |
| ILMN_2705878 | LIMD2 | 1.107359113 | 0.000299498 | 0.014892062 | 0.000299498 |
| ILMN_1227100 | E130112E08RIK | 1.107230432 | 0.002018395 | 0.042006944 | 0.002768024 |
| ILMN_1254814 | CLK1 | 1.10722634 | 0.000942625 | 0.026071536 | 0.000942625 |
| ILMN_2986458 | TERF2IP | 1.107174717 | 0.000299498 | 0.014892062 | 0.000299498 |
| ILMN_1239729 | KIF1B | 1.106854993 | 0.000639896 | 0.021153497 | 0.000639896 |
| ILMN_1217480 | LOC100046080 | 1.106833133 | 0.006967815 | 0.091154627 | 0.004907577 |
| ILMN_2652857 | IFI47 | 1.106721599 | 0.009432307 | 0.108259469 | 0.007644699 |
| ILMN_1257107 | LOC100043821 | 1.106674621 | 0.000639896 | 0.021153497 | 0.000639896 |
| ILMN_2636339 | IRF9 | 1.106644992 | 0.002520973 | 0.048392748 | 0.000942625 |
| ILMN_2479848 | SIF1 | 1.106503824 | 0.000299498 | 0.014892062 | 0.000299498 |
| ILMN_1226514 | GAB3 | 1.106492684 | 0.000299498 | 0.014892062 | 0.000299498 |
| ILMN_3141048 | SEPP1 | 1.106305884 | 0.001496892 | 0.034781534 | 0.000942625 |
| ILMN_2635871 | 3110057O12RIK | 1.106105831 | 0.000639896 | 0.021153497 | 0.000639896 |
| ILMN_2829625 | NTNG1 | 1.106001213 | 0.001225688 | 0.030541554 | 0.001225688 |
| ILMN_2635132 | FOXP3 | 1.105736567 | 0.000639896 | 0.021153497 | 0.000299498 |
| ILMN_1225109 | 2310047C21RIK | 1.105585656 | 0.000942625 | 0.026071536 | 0.000942625 |
| ILMN_1228653 | ZBP1 | 1.105391357 | 0.008987421 | 0.105475934 | 0.00809366 |
| ILMN_1236585 | LOC634213 | 1.105197029 | 0.003253905 | 0.056782136 | 0.001496892 |
| ILMN_1227803 | LOC674707 | 1.105118227 | 0.000639896 | 0.021153497 | 0.000942625 |
| ILMN_2706631 | LOC100048076 | 1.105000545 | 0.000299498 | 0.014892062 | 0.000299498 |
| ILMN_2949605 | UBAC2 | 1.104949833 | 0.000299498 | 0.014892062 | 0.000299498 |
| ILMN_2651054 | LOC100047173 | 1.104947245 | 0.001225688 | 0.030541554 | 0.000942625 |
| ILMN_2691192 | RBBP7 | 1.104851088 | 0.007419532 | 0.094878068 | 0.011860136 |
| ILMN_2569336 | A130024J05RIK | 1.104817015 | 0.009654071 | 0.110018185 | 0.010318182 |
| ILMN_1222346 | A630097D09RIK | 1.104807842 | 0.000299498 | 0.014892062 | 0.000299498 |
| ILMN_1255419 | ZFPN1A1 | 1.10475855 | 0.001225688 | 0.030541554 | 0.001225688 |
| ILMN_2704822 | ACAA2 | 1.104751617 | 0.00227139 | 0.045458063 | 0.00227139 |
| ILMN_2595967 | LOC100039532 | 1.104726083 | 0.000299498 | 0.014892062 | 0.000299498 |
| ILMN_2628174 | ACSS1 | 1.104609544 | 0.000299498 | 0.014892062 | 0.000299498 |
| ILMN_2457324 | TSSC1 | 1.10460787 | 0.000942625 | 0.026071536 | 0.000639896 |
| ILMN_2631994 | HEXIM1 | 1.104522651 | 0.000299498 | 0.014892062 | 0.000299498 |
| ILMN_2544890 | PDE4B | 1.104516163 | 0.002768024 | 0.051337774 | 0.003253905 |
| ILMN_2696491 | PHF11 | 1.104484414 | 0.004206115 | 0.066690827 | 0.002520973 |
| ILMN_2775098 | CYB5 | 1.104384533 | 0.000299498 | 0.014892062 | 0.000299498 |
| ILMN_2880467 | LASS4 | 1.104325447 | 0.00227139 | 0.045458063 | 0.001760487 |
| ILMN_2812244 | MPPE1 | 1.104198108 | 0.000299498 | 0.014892062 | 0.000299498 |
| ILMN_3133748 | GAB3 | 1.1040901 | 0.001496892 | 0.034781534 | 0.001225688 |
| ILMN_2518828 | ZNF24 | 1.104037027 | 0.000639896 | 0.021153497 | 0.000299498 |
| ILMN_1258965 | TMEM66 | 1.10397338 | 0.000942625 | 0.026071536 | 0.000942625 |
| ILMN_1237886 | ENC1 | 1.103956093 | 0.000639896 | 0.021153497 | 0.000299498 |
| ILMN_2492102 | LOC630408 | 1.103768241 | 0.000639896 | 0.021153497 | 0.000299498 |
| ILMN_2710166 | DDX3X | 1.103764891 | 0.005139198 | 0.076197614 | 0.004674836 |
| ILMN_2742928 | FXYD5 | 1.103662574 | 0.019874198 | 0.164084265 | 0.014913834 |
| ILMN_3071525 | CD226 | 1.103645914 | 0.003494726 | 0.059081055 | 0.002018395 |
| ILMN_2720820 | A230046K03RIK | 1.103616266 | 0.000299498 | 0.014892062 | 0.000299498 |
| ILMN_1238069 | 4930539E08RIK | 1.103535154 | 0.000299498 | 0.014892062 | 0.000299498 |
| ILMN_1238285 | DCUN1D5 | 1.103527312 | 0.001496892 | 0.034781534 | 0.001496892 |
| ILMN_1234133 | 3632431M01RIK | 1.103406406 | 0.000942625 | 0.026071536 | 0.000639896 |
| ILMN_1217705 | C130002K18RIK | 1.103274712 | 0.008318086 | 0.101656379 | 0.010318182 |
| ILMN_2614966 | RAB27A | 1.103217796 | 0.000299498 | 0.014892062 | 0.000299498 |
| ILMN_1245393 | STXBP3 | 1.103174614 | 0.003970394 | 0.064249 | 0.00227139 |
| ILMN_1239726 | SNAI3 | 1.103168493 | 0.000639896 | 0.021153497 | 0.000639896 |
| ILMN_1223028 | D230020C06RIK | 1.103101789 | 0.000639896 | 0.021153497 | 0.000639896 |
| ILMN_2854878 | NECAP1 | 1.10302603 | 0.000299498 | 0.014892062 | 0.000299498 |
| ILMN_1242802 | RAB6 | 1.103017062 | 0.000942625 | 0.026071536 | 0.000639896 |
| ILMN_2535779 | LOC383706 | 1.102993315 | 0.000639896 | 0.021153497 | 0.000942625 |
| ILMN_1228235 | CTSE | 1.102982206 | 0.000942625 | 0.026071536 | 0.000942625 |
| ILMN_3163163 | A430078G23RIK | 1.102898707 | 0.000639896 | 0.021153497 | 0.000639896 |
| ILMN_2732190 | PBRM1 | 1.102890972 | 0.006058437 | 0.083694614 | 0.003970394 |
| ILMN_1246770 | YBX3 | 1.102889488 | 0.003494726 | 0.059081055 | 0.003012339 |
| ILMN_1250135 | A930005H10RIK | 1.102857125 | 0.000639896 | 0.021153497 | 0.000639896 |
| ILMN_3114641 | PIK3R1 | 1.102785193 | 0.000299498 | 0.014892062 | 0.000299498 |
| ILMN_2677056 | LOC100048622 | 1.102768087 | 0.002018395 | 0.042006944 | 0.002018395 |
| ILMN_1243042 | LOC381214 | 1.102758352 | 0.000299498 | 0.014892062 | 0.000299498 |
| ILMN_2917338 | RNF125 | 1.102623511 | 0.003733327 | 0.06184411 | 0.003253905 |
| ILMN_2433964 | GIGYF1 | 1.102596985 | 0.001760487 | 0.038593802 | 0.001496892 |
| ILMN_2566477 | C730026O12RIK | 1.102585731 | 0.008541654 | 0.103057212 | 0.01076004 |
| ILMN_2704823 | ACAA2 | 1.102584401 | 0.001760487 | 0.038593802 | 0.001225688 |
| ILMN_1249863 | SLAMF1 | 1.102358687 | 0.001496892 | 0.034781534 | 0.000942625 |
| ILMN_2617820 | PPP3CC | 1.10232076 | 0.000299498 | 0.014892062 | 0.000299498 |
| ILMN_1238109 | PP11R | 1.102299387 | 0.000299498 | 0.014892062 | 0.000299498 |
| ILMN_1257463 | BCL11A | 1.102238843 | 0.000942625 | 0.026071536 | 0.000942625 |
| ILMN_2634157 | ZFP654 | 1.102206279 | 0.000299498 | 0.014892062 | 0.000299498 |
| ILMN_1238869 | LOC278666 | 1.102138175 | 0.004674836 | 0.071504777 | 0.002768024 |
| ILMN_1215910 | FOXN2 | 1.102070233 | 0.000299498 | 0.014892062 | 0.000299498 |
| ILMN_2621086 | FLI1 | 1.102009965 | 0.001225688 | 0.030541554 | 0.001496892 |
| ILMN_2699531 | RGS10 | 1.101989172 | 0.00227139 | 0.045458063 | 0.001496892 |
| ILMN_2603187 | MECP2 | 1.101917508 | 0.000299498 | 0.014892062 | 0.000299498 |
| ILMN_2982781 | KCNK5 | 1.101765886 | 0.000639896 | 0.021153497 | 0.000639896 |
| ILMN_1244845 | NARF | 1.101738476 | 0.000942625 | 0.026071536 | 0.000942625 |
| ILMN_2680398 | ZC3H12D | 1.10146721 | 0.000299498 | 0.014892062 | 0.000299498 |
| ILMN_2605031 | E330018D03RIK | 1.101463118 | 0.005600396 | 0.080174568 | 0.008318086 |
| ILMN_2617477 | 8030476J24 | 1.101261776 | 0.001225688 | 0.030541554 | 0.001225688 |
| ILMN_2901284 | ADD3 | 1.101189602 | 0.000299498 | 0.014892062 | 0.000299498 |
| ILMN_2506499 | UBE3A | 1.101180473 | 0.002520973 | 0.048392748 | 0.002520973 |
| ILMN_2605960 | RAB19 | 1.101133403 | 0.002018395 | 0.042006944 | 0.001496892 |
| ILMN_2562725 | C730013O11RIK | 1.101075632 | 0.000942625 | 0.026071536 | 0.000942625 |
| ILMN_2567626 | PUM1 | 1.101065176 | 0.006286464 | 0.085736353 | 0.005600396 |
| ILMN_2954868 | OASL2 | 1.101052773 | 0.00227139 | 0.045458063 | 0.003012339 |
| ILMN_1233894 | DTNB | 1.100978861 | 0.000299498 | 0.014892062 | 0.000299498 |
| ILMN_1229245 | INPP4B | 1.100970899 | 0.00227139 | 0.045458063 | 0.001760487 |
| ILMN_1256639 | CD247 | 1.100911079 | 0.000639896 | 0.021153497 | 0.000299498 |
| ILMN_2631192 | VPS26B | 1.100779486 | 0.004674836 | 0.071504777 | 0.00227139 |
| ILMN_2038768 | control_ILMN_2038768 | 1.1007468 | 0.109264287 | 0.401166936 | 0.107213169 |
| ILMN_2668977 | 1500032D16RIK | 1.100684878 | 0.000299498 | 0.014892062 | 0.000299498 |
| ILMN_1248211 | SIDT1 | 1.100585537 | 0.003012339 | 0.054401128 | 0.002018395 |
| ILMN_2534151 | IRGB10 | 1.100553681 | 0.005370369 | 0.078183323 | 0.006286464 |
| ILMN_1224211 | EPB4.1L2 | 1.100521767 | 0.005139198 | 0.076197614 | 0.006967815 |
| ILMN_2771182 | H2-Q8 | 1.100477595 | 0.002520973 | 0.048392748 | 0.001760487 |
| ILMN_2890357 | 2610027C15RIK | 1.10037148 | 0.000639896 | 0.021153497 | 0.000639896 |
| ILMN_2705732 | INADL | 1.100280563 | 0.000942625 | 0.026071536 | 0.001225688 |
| ILMN_2422023 | C330006D17RIK | 1.10024538 | 0.003970394 | 0.064249 | 0.005139198 |
| ILMN_1239398 | LOC100046817 | 1.100205887 | 0.020945534 | 0.169933229 | 0.023722035 |
| ILMN_1256245 | RLF | 1.100181996 | 0.005370369 | 0.078183323 | 0.007869606 |
| ILMN_1243846 | NRF1 | 1.100170193 | 0.001225688 | 0.030541554 | 0.001496892 |
| ILMN_1242769 | AKAP8L | 1.100071536 | 0.003494726 | 0.059081055 | 0.003494726 |
| ILMN_2993109 | DDIT4 | 1.100038813 | 0.000299498 | 0.014892062 | 0.000299498 |
| ILMN_1245987 | HNRNPK | 1.100037736 | 0.019659544 | 0.162924549 | 0.028394902 |

Probes with |Fold Change| <1.10 not displayed

| | | | | | |
|---|---|---|---|---|---|
| ILMN_2715198 | GOLGA2 | −1.100056844 | 0.007194081 | 0.092947182 | 0.007644699 |
| ILMN_2711045 | SIRT5 | −1.100175511 | 0.000639896 | 0.021153497 | 0.000942625 |
| ILMN_2734856 | A930009M04RIK | −1.10024481 | 0.000299498 | 0.014892062 | 0.000299498 |
| ILMN_1253854 | TGIF1 | −1.100335725 | 0.010980731 | 0.118109454 | 0.008987421 |
| ILMN_2769945 | BC014795 | −1.10036431 | 0.000942625 | 0.026071536 | 0.000942625 |
| ILMN_2954195 | SCYL1 | −1.100565484 | 0.00809366 | 0.100146322 | 0.006514138 |
| ILMN_2988572 | EZH2 | −1.100604827 | 0.006286464 | 0.085736353 | 0.006058437 |
| ILMN_2728118 | RRP12 | −1.100673522 | 0.000299498 | 0.014892062 | 0.000299498 |
| ILMN_1235470 | HNRNPC | −1.100796556 | 0.000299498 | 0.014892062 | 0.000299498 |
| ILMN_2774410 | STFA1 | −1.100797564 | 0.033675597 | 0.219136594 | 0.03956153 |
| ILMN_2856095 | ZC3H12A | −1.101004577 | 0.001496892 | 0.034781534 | 0.000942625 |
| ILMN_2985447 | CMTM7 | −1.101081672 | 0.000942625 | 0.026071536 | 0.000942625 |
| ILMN_2870487 | CPNE2 | −1.101235208 | 0.003012339 | 0.054401128 | 0.001760487 |
| ILMN_2736762 | IFRD2 | −1.101280587 | 0.009432307 | 0.108259469 | 0.008987421 |
| ILMN_2620061 | TBCB | −1.101312875 | 0.000299498 | 0.014892062 | 0.000299498 |
| ILMN_1252016 | ABHD2 | −1.101327755 | 0.001760487 | 0.038593802 | 0.000639896 |
| ILMN_2683802 | NUDT9 | −1.101534614 | 0.000299498 | 0.014892062 | 0.000299498 |
| ILMN_2758087 | CCNT1 | −1.101561438 | 0.003494726 | 0.059081055 | 0.003253905 |
| ILMN_1222246 | ADFP | −1.101682242 | 0.009432307 | 0.108259469 | 0.008541654 |
| ILMN_1250149 | DNMT3L | −1.101692032 | 0.000942625 | 0.026071536 | 0.000299498 |
| ILMN_2855423 | UBE3B | −1.101823534 | 0.004674836 | 0.071504777 | 0.004441009 |
| ILMN_3093150 | NPM3 | −1.101881244 | 0.01076004 | 0.116961528 | 0.012078865 |
| ILMN_2887208 | RAB1B | −1.1018898 | 0.007419532 | 0.094878068 | 0.004674836 |
| ILMN_1246622 | 9130204C11RIK | −1.102206651 | 0.000942625 | 0.026071536 | 0.000639896 |
| ILMN_1237507 | DLST | −1.102242835 | 0.000299498 | 0.014892062 | 0.000299498 |
| ILMN_2615739 | GM459 | −1.102378757 | 0.017508946 | 0.15321051 | 0.021159798 |
| ILMN_2605767 | REPIN1 | −1.102407679 | 0.000299498 | 0.014892062 | 0.000299498 |
| ILMN_2675033 | SLC9A7 | −1.102560823 | 0.000299498 | 0.014892062 | 0.000299498 |
| ILMN_2531520 | GM962 | −1.10277933 | 0.000639896 | 0.021153497 | 0.000639896 |
| ILMN_2952587 | INTS5 | −1.102916647 | 0.014696493 | 0.139447069 | 0.012735944 |
| ILMN_3162081 | WDR6 | −1.103000504 | 0.001496892 | 0.034781534 | 0.002018395 |
| ILMN_2823778 | SC4MOL | −1.103118657 | 0.000942625 | 0.026071536 | 0.000299498 |
| ILMN_2637804 | 4933407P14RIK | −1.103123645 | 0.027759492 | 0.197119517 | 0.028818751 |
| ILMN_2944939 | CAMK1 | −1.103291951 | 0.000299498 | 0.014892062 | 0.000299498 |
| ILMN_2966386 | RCL1 | −1.10330649 | 0.000639896 | 0.021153497 | 0.000639896 |
| ILMN_2721208 | MSTO1 | −1.103313852 | 0.007644699 | 0.096726012 | 0.005139198 |
| ILMN_1243107 | CENTD3 | −1.103383796 | 0.000639896 | 0.021153497 | 0.000639896 |
| ILMN_2491232 | USP36 | −1.103389463 | 0.002018395 | 0.042006944 | 0.001496892 |
| ILMN_1259100 | PILRB1 | −1.103436708 | 0.004907577 | 0.073992984 | 0.003012339 |
| ILMN_1225747 | ZNFN1A3 | −1.103489414 | 0.000299498 | 0.014892062 | 0.000299498 |
| ILMN_2721149 | ARL11 | −1.103567882 | 0.003253905 | 0.056782136 | 0.003494726 |
| ILMN_1244424 | ADORA2B | −1.103675196 | 0.004206115 | 0.066690827 | 0.002520973 |
| ILMN_1228832 | NGP | −1.103786521 | 0.012954349 | 0.129730009 | 0.01076004 |
| ILMN_1260456 | HSD3B7 | −1.103805679 | 0.000299498 | 0.014892062 | 0.000299498 |
| ILMN_1227235 | TIMM10 | −1.103984846 | 0.001225688 | 0.030541554 | 0.001225688 |
| ILMN_2871749 | EAR6 | −1.10402136 | 0.003253905 | 0.056782136 | 0.004907577 |
| ILMN_2911344 | PLSCR1 | −1.104115255 | 0.000299498 | 0.014892062 | 0.000299498 |
| ILMN_2698494 | NFU1 | −1.104319371 | 0.001225688 | 0.030541554 | 0.001225688 |
| ILMN_2603898 | CLEC4B1 | −1.104355951 | 0.001496892 | 0.034781534 | 0.000639896 |
| ILMN_2749448 | PLEKHO2 | −1.104536512 | 0.001496892 | 0.034781534 | 0.001496892 |
| ILMN_2631014 | PIAS3 | −1.104655312 | 0.000299498 | 0.014892062 | 0.000299498 |
| ILMN_2595863 | CHST10 | −1.10468852 | 0.000299498 | 0.014892062 | 0.000299498 |
| ILMN_1231439 | AATK | −1.104893386 | 0.00227139 | 0.045458063 | 0.001496892 |
| ILMN_2923607 | PHLDA3 | −1.105141716 | 0.000639896 | 0.021153497 | 0.000299498 |
| ILMN_2544343 | STAB1 | −1.105676499 | 0.000299498 | 0.014892062 | 0.000299498 |
| ILMN_1238102 | IDE | −1.105690099 | 0.049597708 | 0.270471422 | 0.05750633 |
| ILMN_2834198 | SCN1B | −1.106256844 | 0.000299498 | 0.014892062 | 0.000639896 |
| ILMN_1236702 | LILRB4 | −1.10637923 | 0.01076004 | 0.116961528 | 0.008987421 |
| ILMN_2979639 | H2-DMB2 | −1.106407472 | 0.00227139 | 0.045458063 | 0.002018395 |
| ILMN_1256817 | SLPI | −1.106517473 | 0.043959273 | 0.252131434 | 0.030087942 |
| ILMN_2590350 | UBE2F | −1.106602174 | 0.000942625 | 0.026071536 | 0.000942625 |
| ILMN_1240318 | SLC7A7 | −1.106705276 | 0.000299498 | 0.014892062 | 0.000299498 |
| ILMN_1215893 | ALG3 | −1.106724214 | 0.000299498 | 0.014892062 | 0.000299498 |
| ILMN_2780915 | PRMT7 | −1.106850835 | 0.001496892 | 0.034781534 | 0.001225688 |
| ILMN_2746830 | BC022224 | −1.106855817 | 0.003970394 | 0.064249 | 0.002018395 |
| ILMN_3120014 | SRI | −1.106861448 | 0.008764316 | 0.104351101 | 0.010539193 |
| ILMN_2959292 | UPP1 | −1.107047972 | 0.006741497 | 0.089431865 | 0.008318086 |
| ILMN_2633457 | 1110057K04RIK | −1.107066336 | 0.000639896 | 0.021153497 | 0.000639896 |
| ILMN_2646209 | CDK10 | −1.107106695 | 0.011640032 | 0.121845949 | 0.009654071 |
| ILMN_2680440 | ATP6V1B2 | −1.107126066 | 0.001225688 | 0.030541554 | 0.000942625 |
| ILMN_2691706 | SAC3D1 | −1.107157088 | 0.00227139 | 0.045458063 | 0.002018395 |
| ILMN_1241909 | ATL3 | −1.107229662 | 0.001760487 | 0.038593802 | 0.000299498 |
| ILMN_2686975 | FAM129B | −1.107251043 | 0.000942625 | 0.026071536 | 0.000639896 |
| ILMN_2900557 | KIF15 | −1.107418991 | 0.005600396 | 0.080174568 | 0.002520973 |
| ILMN_2745954 | 1110007L15RIK | −1.107513874 | 0.000639896 | 0.021153497 | 0.000299498 |
| ILMN_1226372 | RFFL | −1.107562515 | 0.000942625 | 0.026071536 | 0.000639896 |
| ILMN_3084818 | NR2C2AP | −1.107652217 | 0.00227139 | 0.045458063 | 0.001225688 |
| ILMN_1248733 | PA2G4 | −1.107667438 | 0.001760487 | 0.038593802 | 0.002520973 |
| ILMN_2630605 | FSCN1 | −1.107768152 | 0.00809366 | 0.100146322 | 0.006058437 |
| ILMN_3003631 | PPAP2C | −1.10780952 | 0.002768024 | 0.051337774 | 0.002768024 |
| ILMN_2598775 | STARD3NL | −1.107943892 | 0.001760487 | 0.038593802 | 0.001496892 |
| ILMN_2588995 | RAB8B | −1.108140051 | 0.004674836 | 0.071504777 | 0.002018395 |
| ILMN_2936468 | 5730596K20RIK | −1.108252625 | 0.000299498 | 0.014892062 | 0.000299498 |
| ILMN_2699522 | DYRK3 | −1.108291839 | 0.000299498 | 0.014892062 | 0.000299498 |
| ILMN_2611431 | PPP3CA | −1.108544093 | 0.019015163 | 0.15981086 | 0.023082193 |
| ILMN_2908056 | TMBIM1 | −1.108696543 | 0.000299498 | 0.014892062 | 0.000299498 |
| ILMN_2744909 | GMPPB | −1.108708169 | 0.000639896 | 0.021153497 | 0.000639896 |
| ILMN_2925281 | ABCD3 | −1.108715146 | 0.000299498 | 0.014892062 | 0.000299498 |
| ILMN_2988931 | STFA1 | −1.108889714 | 0.014262117 | 0.136875566 | 0.016213806 |
| ILMN_2819679 | TMEM50A | −1.108893575 | 0.016645639 | 0.148953717 | 0.018586049 |
| ILMN_2698430 | BCL2L1 | −1.109012645 | 0.000942625 | 0.026071536 | 0.000942625 |
| ILMN_3137980 | ZFP41 | −1.109014707 | 0.000299498 | 0.014892062 | 0.000299498 |
| ILMN_2914507 | NDUFB6 | −1.109148444 | 0.006286464 | 0.085736353 | 0.006286464 |
| ILMN_1243329 | DAB2 | −1.109426935 | 0.011860136 | 0.12340942 | 0.00809366 |
| ILMN_2640453 | WDR55 | −1.109433931 | 0.000639896 | 0.021153497 | 0.000639896 |
| ILMN_2867696 | CDC42BPB | −1.110032665 | 0.000299498 | 0.014892062 | 0.000299498 |
| ILMN_2795412 | TMEM176A | −1.110201644 | 0.000639896 | 0.021153497 | 0.000639896 |
| ILMN_2727273 | SWAP70 | −1.110366306 | 0.000299498 | 0.014892062 | 0.000299498 |
| ILMN_2620767 | ABHD2 | −1.110517436 | 0.000299498 | 0.014892062 | 0.000299498 |
| ILMN_1221920 | PLEKHG3 | −1.110664539 | 0.000299498 | 0.014892062 | 0.000299498 |
| ILMN_2764607 | 1810009N02RIK | −1.110668063 | 0.000299498 | 0.014892062 | 0.000299498 |
| ILMN_2842767 | NACC1 | −1.110682075 | 0.000639896 | 0.021153497 | 0.000639896 |
| ILMN_2755399 | PPP2R4 | −1.110682315 | 0.002768024 | 0.051337774 | 0.00227139 |
| ILMN_1256354 | GCLM | −1.110765414 | 0.001496892 | 0.034781534 | 0.000942625 |
| ILMN_2981689 | NUP133 | −1.11080026 | 0.000299498 | 0.014892062 | 0.000299498 |
| ILMN_2915303 | TREM3 | −1.110935528 | 0.000942625 | 0.026071536 | 0.001225688 |
| ILMN_1215209 | CLECSF9 | −1.110943822 | 0.019874198 | 0.164084265 | 0.020303308 |
| ILMN_2841290 | TNFAIP2 | −1.110987512 | 0.000299498 | 0.014892062 | 0.000639896 |
| ILMN_2959293 | UPP1 | −1.111007524 | 0.001496892 | 0.034781534 | 0.001496892 |
| ILMN_2614563 | APOL7C | −1.111021044 | 0.000299498 | 0.014892062 | 0.000299498 |
| ILMN_3160203 | ZFP213 | −1.11102856 | 0.000639896 | 0.021153497 | 0.000639896 |
| ILMN_2601379 | MGC18837 | −1.111045685 | 0.000299498 | 0.014892062 | 0.000299498 |
| ILMN_1235000 | YIF1A | −1.111117156 | 0.000639896 | 0.021153497 | 0.000639896 |
| ILMN_2640995 | SPIRE2 | −1.111127251 | 0.000299498 | 0.014892062 | 0.000299498 |
| ILMN_2884098 | GORASP2 | −1.111288278 | 0.000299498 | 0.014892062 | 0.000299498 |
| ILMN_1256142 | MARCKS | −1.111356803 | 0.005370369 | 0.078183323 | 0.001760487 |
| ILMN_2601147 | KDELC1 | −1.111368149 | 0.001760487 | 0.038593802 | 0.002018395 |
| ILMN_2680549 | PIK3CB | −1.111474769 | 0.000299498 | 0.014892062 | 0.000299498 |
| ILMN_1218181 | IFITM6 | −1.111515015 | 0.001225688 | 0.030541554 | 0.001225688 |
| ILMN_2550095 | 6230425C21RIK | −1.111625769 | 0.001496892 | 0.034781534 | 0.001760487 |
| ILMN_1224390 | 1700129I04RIK | −1.111956114 | 0.000299498 | 0.014892062 | 0.000299498 |
| ILMN_1240746 | MCPT4 | −1.111968534 | 0.004441009 | 0.0691859 | 0.001760487 |
| ILMN_1230423 | SH3PXD2B | −1.111999219 | 0.000299498 | 0.014892062 | 0.000299498 |
| ILMN_1247468 | FTSJ3 | −1.112004157 | 0.000299498 | 0.014892062 | 0.000299498 |
| ILMN_3140913 | TBC1D2 | −1.112150855 | 0.000299498 | 0.014892062 | 0.000299498 |
| ILMN_2519673 | VWF | −1.112341924 | 0.000639896 | 0.021153497 | 0.000299498 |
| ILMN_2617468 | CHAC1 | −1.112462827 | 0.001225688 | 0.030541554 | 0.000942625 |
| ILMN_2450031 | SCL0004020.1_31 | −1.112588927 | 0.000942625 | 0.026071536 | 0.000942625 |
| ILMN_2450155 | IGLC2_J00595_IG_LAMBDA_CONSTANT_2_14 | −1.112608245 | 0.000299498 | 0.014892062 | 0.000299498 |
| ILMN_1218104 | 5630401D24RIK | −1.112744368 | 0.000299498 | 0.014892062 | 0.000299498 |
| ILMN_1237743 | LOC382813 | −1.112766578 | 0.000299498 | 0.014892062 | 0.000299498 |
| ILMN_1222872 | WBSCR16 | −1.112770151 | 0.003733327 | 0.06184411 | 0.003970394 |
| ILMN_2687828 | ARID3A | −1.112982962 | 0.009654071 | 0.110018185 | 0.007869606 |
| ILMN_2772492 | TSR1 | −1.11339113 | 0.000299498 | 0.014892062 | 0.000299498 |
| ILMN_2868480 | EAR4 | −1.11346403 | 0.068288049 | 0.318497497 | 0.073665179 |
| ILMN_1219168 | GPN1 | −1.113513745 | 0.000299498 | 0.014892062 | 0.000299498 |
| ILMN_1214608 | PLXND1 | −1.113701478 | 0.000942625 | 0.026071536 | 0.000639896 |
| ILMN_2723708 | FNBP1 | −1.113872343 | 0.000299498 | 0.014892062 | 0.000299498 |
| ILMN_2806180 | CSF3R | −1.11399081 | 0.001760487 | 0.038593802 | 0.001496892 |
| ILMN_2652172 | NAGK | −1.114167208 | 0.000942625 | 0.026071536 | 0.000942625 |
| ILMN_2859847 | PYGL | −1.114288643 | 0.001496892 | 0.034781534 | 0.001496892 |
| ILMN_2470360 | BC037704 | −1.11448614 | 0.000299498 | 0.014892062 | 0.000299498 |
| ILMN_1217040 | 1300001I01RIK | −1.114584244 | 0.002768024 | 0.051337774 | 0.003494726 |
| ILMN_1223285 | HSPA2 | −1.115046275 | 0.000299498 | 0.014892062 | 0.000299498 |
| ILMN_2950814 | GM962 | −1.115047143 | 0.006741497 | 0.089431865 | 0.005370369 |
| ILMN_1214952 | CLN3 | −1.115097709 | 0.000299498 | 0.014892062 | 0.000299498 |
| ILMN_3163504 | METTL4 | −1.115151964 | 0.000299498 | 0.014892062 | 0.000299498 |
| ILMN_2822552 | UBTD2 | −1.115214016 | 0.000942625 | 0.026071536 | 0.001225688 |
| ILMN_2717678 | MUC13 | −1.115246667 | 0.000639896 | 0.021153497 | 0.000299498 |
| ILMN_2433547 | TRP53BP1 | −1.115309394 | 0.000299498 | 0.014892062 | 0.000299498 |
| ILMN_2788073 | HMOX1 | −1.115323968 | 0.006967815 | 0.091154627 | 0.003970394 |
| ILMN_1219208 | NOL6 | −1.115717769 | 0.000299498 | 0.014892062 | 0.000299498 |
| ILMN_1237430 | PHLPP | −1.11586422 | 0.001225688 | 0.030541554 | 0.001225688 |
| ILMN_2723483 | POLR1B | −1.115874714 | 0.001225688 | 0.030541554 | 0.000639896 |
| ILMN_2958484 | NLRX1 | −1.115901939 | 0.000639896 | 0.021153497 | 0.000639896 |
| ILMN_2661367 | HYOU1 | −1.11593221 | 0.000639896 | 0.021153497 | 0.000639896 |
| ILMN_2608145 | HDHD3 | −1.116104843 | 0.000299498 | 0.014892062 | 0.000299498 |
| ILMN_2791028 | APP | −1.116183137 | 0.002768024 | 0.051337774 | 0.002768024 |
| ILMN_2524865 | CHI3L4 | −1.11643408 | 0.005600396 | 0.080174568 | 0.004206115 |
| ILMN_1254929 | RCL1 | −1.116461155 | 0.000299498 | 0.014892062 | 0.000299498 |
| ILMN_2939185 | BC056474 | −1.116523763 | 0.000299498 | 0.014892062 | 0.000299498 |
| ILMN_1228613 | PYCRL | −1.116634512 | 0.000299498 | 0.014892062 | 0.000299498 |
| ILMN_2877507 | PIGQ | −1.11667927 | 0.000942625 | 0.026071536 | 0.000639896 |
| ILMN_2868131 | PIGT | −1.117024219 | 0.001225688 | 0.030541554 | 0.001225688 |
| ILMN_1221943 | SDF2L1 | −1.117198955 | 0.003012339 | 0.054401128 | 0.002018395 |
| ILMN_2750515 | FOS | −1.11747226 | 0.000299498 | 0.014892062 | 0.000299498 |
| ILMN_3005873 | SORT1 | −1.11753372 | 0.000299498 | 0.014892062 | 0.000299498 |
| ILMN_2934448 | TJAP1 | −1.118088782 | 0.000299498 | 0.014892062 | 0.000299498 |
| ILMN_1240615 | OLFM1 | −1.118125635 | 0.002018395 | 0.042006944 | 0.001225688 |
| ILMN_2706231 | SYNGR2 | −1.118544974 | 0.002018395 | 0.042006944 | 0.00227139 |
| ILMN_2776850 | GAS7 | −1.118567525 | 0.000639896 | 0.021153497 | 0.000639896 |
| ILMN_2815889 | GRWD1 | −1.118687936 | 0.000299498 | 0.014892062 | 0.000299498 |
| ILMN_1258394 | AARS | −1.118724378 | 0.000299498 | 0.014892062 | 0.000299498 |
| ILMN_2685565 | TYMP | −1.119134268 | 0.000299498 | 0.014892062 | 0.000299498 |
| ILMN_1249313 | RABL3 | −1.119280075 | 0.000299498 | 0.014892062 | 0.000299498 |
| ILMN_2630459 | CXCR4 | −1.119486645 | 0.016645639 | 0.148953717 | 0.010096998 |
| ILMN_2736690 | 2210411K11RIK | −1.119517225 | 0.000299498 | 0.014892062 | 0.000299498 |
| ILMN_2987863 | PER2 | −1.119751625 | 0.002018395 | 0.042006944 | 0.001225688 |
| ILMN_2600744 | RGS16 | −1.119945836 | 0.000639896 | 0.021153497 | 0.000299498 |
| ILMN_3091641 | DAB2 | −1.120084423 | 0.005600396 | 0.080174568 | 0.003494726 |
| ILMN_2684651 | MSI2H | −1.120151943 | 0.000639896 | 0.021153497 | 0.000942625 |
| ILMN_2425415 | UBE3B | −1.12020859 | 0.000299498 | 0.014892062 | 0.000299498 |
| ILMN_2733733 | TLR2 | −1.120344381 | 0.001760487 | 0.038593802 | 0.001496892 |
| ILMN_2855261 | SLC25A39 | −1.120465978 | 0.000299498 | 0.014892062 | 0.000299498 |
| ILMN_2713464 | ABHD4 | −1.120638738 | 0.000299498 | 0.014892062 | 0.000299498 |
| ILMN_2574165 | SLC7A11 | −1.120754955 | 0.000299498 | 0.014892062 | 0.000299498 |
| ILMN_1259069 | F730003H07RIK | −1.121065354 | 0.022015007 | 0.174899503 | 0.015131078 |
| ILMN_1252157 | A330102K04RIK | −1.121227393 | 0.000639896 | 0.021153497 | 0.000639896 |
| ILMN_2935032 | FBXL6 | −1.121239133 | 0.000299498 | 0.014892062 | 0.000299498 |
| ILMN_1253919 | CCL3 | −1.121443309 | 0.009654071 | 0.110018185 | 0.009654071 |
| ILMN_1260585 | STFA2 | −1.121655647 | 0.000942625 | 0.026071536 | 0.000639896 |
| ILMN_3136638 | SNCA | −1.121686063 | 0.007869606 | 0.098427373 | 0.003733327 |
| ILMN_2936380 | SGPL1 | −1.121901135 | 0.000299498 | 0.014892062 | 0.000299498 |
| ILMN_2673211 | PFN1 | −1.121964524 | 0.004674836 | 0.071504777 | 0.001760487 |
| ILMN_1221451 | DHRSX | −1.12290417 | 0.001760487 | 0.038593802 | 0.001760487 |
| ILMN_2450491 | TRFR2 | −1.122926835 | 0.000299498 | 0.014892062 | 0.000299498 |
| ILMN_2790512 | 1110039B18RIK | −1.123368404 | 0.000299498 | 0.014892062 | 0.000299498 |
| ILMN_2894450 | SNX15 | −1.123418502 | 0.000299498 | 0.014892062 | 0.000299498 |
| ILMN_1223966 | LOC385959 | −1.123693602 | 0.001225688 | 0.030541554 | 0.001496892 |
| ILMN_2736347 | PRMT7 | −1.123726664 | 0.000299498 | 0.014892062 | 0.000299498 |
| ILMN_2491099 | XPOT | −1.123742439 | 0.000299498 | 0.014892062 | 0.000299498 |
| ILMN_1232396 | EAR2 | −1.123772011 | 0.064974747 | 0.311316494 | 0.062694716 |
| ILMN_1218602 | G6PC3 | −1.124251076 | 0.002768024 | 0.051337774 | 0.001760487 |
| ILMN_3135781 | ANXA3 | −1.124627391 | 0.001225688 | 0.030541554 | 0.000639896 |
| ILMN_2892376 | DHX29 | −1.124906344 | 0.000299498 | 0.014892062 | 0.000299498 |
| ILMN_3161878 | BID | −1.124973739 | 0.000299498 | 0.014892062 | 0.000299498 |
| ILMN_2847787 | EMR1 | −1.125484607 | 0.004206115 | 0.066690827 | 0.002768024 |
| ILMN_2646878 | BLMH | −1.12551314 | 0.000299498 | 0.014892062 | 0.000299498 |
| ILMN_2706853 | SCAMP1 | −1.125532389 | 0.000299498 | 0.014892062 | 0.000299498 |
| ILMN_2843698 | SMARCAL1 | −1.125762101 | 0.000299498 | 0.014892062 | 0.000299498 |
| ILMN_2717366 | OASL1 | −1.125975138 | 0.000639896 | 0.021153497 | 0.000299498 |
| ILMN_2546073 | WDR68 | −1.126231678 | 0.000299498 | 0.014892062 | 0.000299498 |
| ILMN_3128907 | CD63 | −1.126420996 | 0.001760487 | 0.038593802 | 0.001496892 |
| ILMN_2678355 | AMIGO2 | −1.126430355 | 0.000299498 | 0.014892062 | 0.000299498 |
| ILMN_2655895 | AKT2 | −1.126825454 | 0.000299498 | 0.014892062 | 0.000299498 |
| ILMN_2625114 | CDKAL1 | −1.127010951 | 0.001225688 | 0.030541554 | 0.000942625 |
| ILMN_2667384 | SLC6A9 | −1.127361604 | 0.000299498 | 0.014892062 | 0.000299498 |
| ILMN_1222988 | LOC386268 | −1.127498485 | 0.000942625 | 0.026071536 | 0.000299498 |
| ILMN_2895557 | GFI1B | −1.127535545 | 0.000639896 | 0.021153497 | 0.000299498 |
| ILMN_2925653 | EAR2 | −1.127584391 | 0.047928871 | 0.265103375 | 0.046049604 |
| ILMN_1216972 | CLEC4E | −1.128106489 | 0.003733327 | 0.06184411 | 0.002018395 |
| ILMN_2652385 | BAZ2A | −1.128274842 | 0.002018395 | 0.042006944 | 0.002018395 |
| ILMN_3158659 | C230096C10RIK | −1.128382209 | 0.001496892 | 0.034781534 | 0.000942625 |
| ILMN_2675223 | CD33 | −1.128563646 | 0.000942625 | 0.026071536 | 0.000639896 |
| ILMN_2609073 | MGAT4B | −1.128660345 | 0.000299498 | 0.014892062 | 0.000299498 |
| ILMN_2588055 | ACTB | −1.128864948 | 0.313064067 | 0.643080133 | 0.323948521 |
| ILMN_2680686 | ALG12 | −1.129043435 | 0.000299498 | 0.014892062 | 0.000299498 |
| ILMN_1219820 | 1200002N14RIK | −1.129152823 | 0.000639896 | 0.021153497 | 0.000639896 |
| ILMN_2589893 | TBRG4 | −1.129261707 | 0.000639896 | 0.021153497 | 0.000639896 |
| ILMN_2975312 | FCER2A | −1.129737894 | 0.002520973 | 0.048392748 | 0.002018395 |
| ILMN_2667033 | ZXDC | −1.130679589 | 0.000299498 | 0.014892062 | 0.000299498 |
| ILMN_2622671 | ACSL1 | −1.131479516 | 0.000639896 | 0.021153497 | 0.000299498 |
| ILMN_2674324 | THOP1 | −1.131537424 | 0.000639896 | 0.021153497 | 0.000639896 |
| ILMN_1259290 | AKT3 | −1.131538979 | 0.000299498 | 0.014892062 | 0.000299498 |
| ILMN_2932539 | RING1 | −1.132133147 | 0.000299498 | 0.014892062 | 0.000299498 |
| ILMN_1253819 | PRKAR2B | −1.132148923 | 0.000299498 | 0.014892062 | 0.000299498 |
| ILMN_2995688 | EG433016 | −1.132494728 | 0.019444821 | 0.1617544 | 0.016429772 |
| ILMN_1238640 | 2310003H01RIK | −1.132552815 | 0.000639896 | 0.021153497 | 0.000639896 |
| ILMN_2696696 | GYPA | −1.132817135 | 0.002768024 | 0.051337774 | 0.001496892 |
| ILMN_1225192 | NFKBID | −1.133066098 | 0.000299498 | 0.014892062 | 0.000299498 |
| ILMN_1243066 | IL1A | −1.133413071 | 0.020088786 | 0.165267027 | 0.014262117 |
| ILMN_2937320 | SCHIP1 | −1.133751669 | 0.000942625 | 0.026071536 | 0.000299498 |
| ILMN_2731237 | D8ERTD82E | −1.134047583 | 0.000299498 | 0.014892062 | 0.000299498 |
| ILMN_2599657 | FMNL3 | −1.134171185 | 0.000299498 | 0.014892062 | 0.000299498 |
| ILMN_2731550 | BRP17 | −1.134397653 | 0.000299498 | 0.014892062 | 0.000299498 |
| ILMN_2762189 | GPATCH1 | −1.134466014 | 0.000299498 | 0.014892062 | 0.000299498 |
| ILMN_2959291 | UPP1 | −1.13449718 | 0.000942625 | 0.026071536 | 0.000942625 |
| ILMN_2841289 | TNFAIP2 | −1.134827216 | 0.000299498 | 0.014892062 | 0.000299493 |
| ILMN_2671411 | KLC1 | −1.135147123 | 0.000299498 | 0.014892062 | 0.000299498 |
| ILMN_2596522 | MT1 | −1.135343672 | 0.000299498 | 0.014892062 | 0.000299498 |
| ILMN_2939681 | LYZS | −1.136569055 | 0.005600396 | 0.080174568 | 0.004441009 |
| ILMN_2964076 | TSPAN31 | −1.137017412 | 0.001225688 | 0.030541554 | 0.000942625 |
| ILMN_2692797 | SLC11A1 | −1.137029044 | 0.000942625 | 0.026071536 | 0.000299498 |
| ILMN_2693124 | RAB1B | −1.137428102 | 0.019659544 | 0.162924549 | 0.011420742 |
| ILMN_1220769 | LMAN2L | −1.138159096 | 0.000299498 | 0.014892062 | 0.000299498 |
| ILMN_2701271 | PLSCR1 | −1.138607055 | 0.000299498 | 0.014892062 | 0.000299498 |
| ILMN_2778289 | CXCL2 | −1.140544603 | 0.026486805 | 0.192885413 | 0.01404445 |
| ILMN_1249362 | LOC386298 | −1.141212883 | 0.001225688 | 0.030541554 | 0.001225688 |
| ILMN_2661287 | AKP2 | −1.141256855 | 0.000299498 | 0.014892062 | 0.000299498 |
| ILMN_2846865 | ACTB | −1.141333782 | 0.27330516 | 0.606575161 | 0.284413487 |
| ILMN_2925711 | DUSP6 | −1.142932506 | 0.000639896 | 0.021153497 | 0.000299498 |
| ILMN_2803674 | S100A9 | −1.1442518 | 0.002018395 | 0.042006944 | 0.000942625 |
| ILMN_2866276 | CSF2RA | −1.144341375 | 0.000299498 | 0.014892062 | 0.000299498 |
| ILMN_2524986 | EAR3 | −1.144901605 | 0.017077844 | 0.151275609 | 0.018586049 |
| ILMN_2479717 | PILRA | −1.145002307 | 0.000299498 | 0.014892062 | 0.000299498 |
| ILMN_2987709 | SLC15A3 | −1.145009054 | 0.000299498 | 0.014892062 | 0.000299498 |
| ILMN_1224736 | ZMIZ1 | −1.145106147 | 0.000299498 | 0.014892062 | 0.000299498 |
| ILMN_3155815 | 9830134C10RIK | −1.145114089 | 0.000299498 | 0.014892062 | 0.000299498 |
| ILMN_2654754 | GP38 | −1.146427658 | 0.008541654 | 0.103057212 | 0.005600396 |
| ILMN_3104139 | RBM12 | −1.147016272 | 0.000299498 | 0.014892062 | 0.000299498 |
| ILMN_2712986 | CHI3L3 | −1.147371605 | 0.001225688 | 0.030541554 | 0.001496892 |
| ILMN_2757368 | CRELD2 | −1.147374056 | 0.000299498 | 0.014892062 | 0.000299498 |
| ILMN_2491202 | VNN3 | −1.148153174 | 0.000299498 | 0.014892062 | 0.000299498 |
| ILMN_1230766 | SCMH1 | −1.148605754 | 0.000299498 | 0.014892062 | 0.000299498 |
| ILMN_2771380 | CD52 | −1.149676808 | 0.008987421 | 0.105475934 | 0.007644699 |
| ILMN_1230708 | CLEC4A1 | −1.149760601 | 0.000299498 | 0.014892062 | 0.000299498 |
| ILMN_2684575 | PCYOX1L | −1.149890332 | 0.001225688 | 0.030541554 | 0.000639896 |
| ILMN_1218347 | MYLK | −1.151233811 | 0.000299498 | 0.014892062 | 0.000299498 |
| ILMN_2601946 | 5033414K04RIK | −1.151524006 | 0.000299498 | 0.014892062 | 0.000299498 |
| ILMN_2835117 | CCL7 | −1.1516614 | 0.000299498 | 0.014892062 | 0.000299498 |
| ILMN_2759484 | C3 | −1.152531108 | 0.000639896 | 0.021153497 | 0.000299498 |
| ILMN_1223416 | FER1L3 | −1.152772279 | 0.000299498 | 0.014892062 | 0.000299498 |
| ILMN_2901626 | TNFRSF21 | −1.153121675 | 0.000942625 | 0.026071536 | 0.000639896 |
| ILMN_2693895 | ACTA2 | −1.153348714 | 0.000299498 | 0.014892062 | 0.000299498 |
| ILMN_2777498 | IL1B | −1.153646647 | 0.005600396 | 0.080174568 | 0.004206115 |
| ILMN_2789900 | CD177 | −1.154670942 | 0.000639896 | 0.021153497 | 0.000299498 |
| ILMN_2618423 | ZXDC | −1.15475865 | 0.000299498 | 0.014892062 | 0.000299498 |
| ILMN_1242457 | FPR2 | −1.155423301 | 0.003012339 | 0.054401128 | 0.002018395 |
| ILMN_2742075 | CD14 | −1.15645531 | 0.003253905 | 0.056782136 | 0.002520973 |
| ILMN_2868220 | INHBA | −1.156681552 | 0.001225688 | 0.030541554 | 0.000942625 |
| ILMN_2674884 | SLC11A1 | −1.157414839 | 0.000942625 | 0.026071536 | 0.000942625 |
| ILMN_3161601 | SNCA | −1.157946415 | 0.002018395 | 0.042006944 | 0.001496892 |
| ILMN_2705860 | TPSAB1 | −1.160383394 | 0.000942625 | 0.026071536 | 0.000639896 |
| ILMN_2710905 | S100A8 | −1.163713512 | 0.000639896 | 0.021153497 | 0.000299498 |
| ILMN_2595732 | LOC100046232 | −1.165021501 | 0.000299498 | 0.014892062 | 0.000299498 |
| ILMN_2719732 | MS4A3 | −1.166305486 | 0.000639896 | 0.021153497 | 0.000299498 |
| ILMN_1221700 | ELA2 | −1.167300103 | 0.000299498 | 0.014892062 | 0.000299498 |
| ILMN_1224472 | CCL4 | −1.167531549 | 0.000942625 | 0.026071536 | 0.000639896 |
| ILMN_2728038 | ARHGAP24 | −1.167825732 | 0.000299498 | 0.014892062 | 0.000299498 |
| ILMN_1252819 | PRMT7 | −1.167997376 | 0.000299498 | 0.014892062 | 0.000299498 |
| ILMN_2538531 | LOC386360 | −1.168408104 | 0.000299498 | 0.014892062 | 0.000299498 |
| ILMN_2493826 | UGT1A10 | −1.169469481 | 0.000299498 | 0.014892062 | 0.000299498 |
| ILMN_2834379 | TGFBI | −1.172758284 | 0.001760487 | 0.038593802 | 0.001225688 |
| ILMN_1222543 | UGT1A10 | −1.173528971 | 0.000299498 | 0.014892062 | 0.000299498 |
| ILMN_2983516 | SLC11A1 | −1.17650384 | 0.001225688 | 0.030541554 | 0.000639896 |
| ILMN_2426480 | 3830612M24 | −1.17764975 | 0.000639896 | 0.021153497 | 0.000299498 |
| ILMN_2592554 | REXO1 | −1.182418789 | 0.000299498 | 0.014892062 | 0.000299498 |
| ILMN_1220548 | LRRC41 | −1.183163213 | 0.000299498 | 0.014892062 | 0.000299498 |
| ILMN_1233336 | LOC386199 | −1.183990269 | 0.000639896 | 0.021153497 | 0.000639896 |
| ILMN_2646985 | ATF5 | −1.18420231 | 0.000299498 | 0.014892062 | 0.000299498 |
| ILMN_2925094 | MPO | −1.185131735 | 0.000299498 | 0.014892062 | 0.000299498 |
| ILMN_1252076 | LYZ2 | −1.187850205 | 0.003494726 | 0.059081055 | 0.001496892 |
| ILMN_2948143 | SLC7A11 | −1.188505133 | 0.000299498 | 0.014892062 | 0.000299498 |
| ILMN_1219230 | LOC100043402 | −1.188859089 | 0.000299498 | 0.014892062 | 0.000299498 |
| ILMN_1230140 | LOC385923 | −1.189899209 | 0.000299498 | 0.014892062 | 0.000299498 |
| ILMN_3162125 | GRM6 | −1.194679101 | 0.000299498 | 0.014892062 | 0.000299498 |
| ILMN_2771176 | CCL7 | −1.196175382 | 0.000299498 | 0.014892062 | 0.000299498 |
| ILMN_2847115 | GPR109A | −1.199553298 | 0.000639896 | 0.021153497 | 0.000639896 |
| ILMN_2758029 | PRTN3 | −1.205025411 | 0.000299498 | 0.014892062 | 0.000299498 |
| ILMN_2722996 | SIRPA | −1.209776171 | 0.000299498 | 0.014892062 | 0.000299498 |
| ILMN_2657828 | RHBDF1 | −1.216435604 | 0.000299498 | 0.014892062 | 0.000299498 |
| ILMN_2712075 | LCN2 | −1.216711224 | 0.000639896 | 0.021153497 | 0.000299498 |
| ILMN_2878071 | LYZ | −1.22864681 | 0.000299498 | 0.014892062 | 0.000299498 |
| ILMN_2705628 | CLEC4D | −1.228745779 | 0.000639896 | 0.021153497 | 0.000639896 |
| ILMN_1233982 | LOC270589 | −1.22932799 | 0.000299498 | 0.014892062 | 0.000299498 |
| ILMN_1377923 | ACTB | −1.230879389 | 0.076350192 | 0.337236501 | 0.050222887 |
| ILMN_1249030 | MPO | −1.231747939 | 0.000299498 | 0.014892062 | 0.000299498 |
| ILMN_2538242 | LOC386144 | −1.239189285 | 0.000299498 | 0.014892062 | 0.000299498 |
| ILMN_2993314 | CLEC4N | −1.244916169 | 0.000639896 | 0.021153497 | 0.000299498 |
| ILMN_2750842 | 2610027L16RIK | −1.245539485 | 0.000299498 | 0.014892062 | 0.000299498 |
| ILMN_2600421 | MPO | −1.29293209 | 0.000299498 | 0.014892062 | 0.000299498 |
| ILMN_2763245 | CXCL1 | −1.3 | 0.000639896 | 0.021153497 | 0.000299498 |
| ILMN_1253874 | SERPINB2 | −1.31 | 0.000299498 | 0.014892062 | 0.000299498 |
| ILMN_1215076 | F830002E14RIK | −1.32 | 0.002018395 | 0.042006944 | 0.001225688 |
| ILMN_2725259 | IL2 | −1.37 | 0.000299498 | 0.014892062 | 0.000299498 |

| | | | | | | Significant | | |
|---|---|---|---|---|---|---|---|---|
| | Comparative Marker | | | | | in | | |
| | Selection | | | | | Parametric | | |
| | SNR | LIMMA | | ANOVA | | Tests | Wilcoxon Rank Sum | |
| Probe | Adj. P. | Nom. P. | Adj. P. | Nom. P. | Adj. P. | 1—yes, 0—no | Nom. P. | Adj. P. |
| ILMN_2685712 | 0.013353714 | 0.000000236 | 0.0000718 | 0.000000401 | 0.000139 | 1 | 2.06483E−05 | 0.004309944 |
| ILMN_2595857 | 0.013353714 | 5.82E−12 | 1.69E−08 | 1.23E−08 | 0.0000119 | 1 | 6.03547E−07 | 0.000573333 |
| ILMN_2791459 | 0.013353714 | 0.00000199 | 0.000375639 | 0.00000642 | 0.00108 | 1 | 0.000197175 | 0.015477107 |
| ILMN_2675232 | 0.013353714 | 7.43E−19 | 3.46E−14 | 5.6E−14 | 2.61E−09 | 1 | 7.95833E−09 | 0.000122224 |
| ILMN_1220788 | 0.013353714 | 3.92E−10 | 0.00000057 | 4.06E−09 | 0.00000573 | 1 | 4.9657E−07 | 0.000525315 |
| ILMN_1253828 | 0.013353714 | 7.18E−09 | 0.00000514 | 0.000000189 | 0.0000836 | 1 | 1.53585E−05 | 0.003592421 |
| ILMN_2856926 | 0.013353714 | 3.75E−15 | 5.82E−11 | 5.66E−11 | 0.000000293 | 1 | 5.89499E−08 | 0.000249449 |
| ILMN_1218037 | 0.013353714 | 1.6E−10 | 0.000000267 | 8.95E−09 | 0.0000105 | 1 | 1.80477E−06 | 0.001090998 |
| ILMN_2664929 | 0.013353714 | 6.15E−13 | 2.6E−09 | 8E−11 | 0.000000367 | 1 | 1.49655E−07 | 0.000345407 |
| ILMN_1240428 | 0.013353714 | 3.36E−10 | 0.000000504 | 3.35E−08 | 0.0000244 | 1 | 2.88347E−07 | 0.000419427 |
| ILMN_2741169 | 0.013353714 | 2.32E−15 | 5.4E−11 | 3.06E−12 | 4.75E−08 | 1 | 1.57497E−07 | 0.000345407 |
| ILMN_1250001 | 0.019410925 | 0.00000259 | 0.000464931 | 0.000033 | 0.003242217 | 1 | 0.000130304 | 0.012203733 |
| ILMN_2611022 | 0.013353714 | 7.52E−10 | 0.000000915 | 0.00000014 | 0.0000707 | 1 | 1.5356E−05 | 0.003592421 |
| ILMN_2707941 | 0.013353714 | 9.31E−14 | 8.67E−10 | 2.22E−12 | 4.75E−08 | 1 | 1.65715E−07 | 0.000345407 |
| ILMN_2769772 | 0.013353714 | 8.08E−12 | 2.21E−08 | 2.26E−09 | 0.00000363 | 1 | 4.9657E−07 | 0.000525315 |
| ILMN_2775055 | 0.013353714 | 3.92E−12 | 0.000000014 | 2.69E−09 | 0.00000405 | 1 | 4.7283E−07 | 0.000525315 |
| ILMN_3128992 | 0.013353714 | 5.34E−13 | 2.48E−09 | 8.67E−11 | 0.000000367 | 1 | 1.10038E−07 | 0.000299615 |
| ILMN_2416460 | 0.013353714 | 5.05E−12 | 1.57E−08 | 2.14E−09 | 0.00000357 | 1 | 2.49646E−06 | 0.001335664 |
| ILMN_2670150 | 0.013353714 | 1.69E−08 | 0.0000104 | 0.000000275 | 0.000109783 | 1 | 1.80477E−06 | 0.001090998 |
| ILMN_2454209 | 0.013353714 | 4.23E−09 | 0.00000323 | 0.00000012 | 0.0000626 | 1 | 0.000103509 | 0.010612415 |
| ILMN_2960700 | 0.013353714 | 0.00000023 | 0.0000712 | 0.00000418 | 0.000771264 | 1 | 3.99889E−05 | 0.006183935 |
| ILMN_1229318 | 0.013353714 | 1.26E−09 | 0.00000128 | 0.000000057 | 0.0000353 | 1 | 1.98091E−06 | 0.001152569 |
| ILMN_1233589 | 0.013353714 | 3.67E−13 | 1.9E−09 | 1.77E−11 | 0.000000121 | 1 | 2.95621E−08 | 0.000229338 |
| ILMN_2678521 | 0.013353714 | 1.33E−13 | 8.87E−10 | 3.83E−11 | 0.000000223 | 1 | 7.26855E−08 | 0.000260253 |
| ILMN_1256701 | 0.013353714 | 2.75E−08 | 0.0000147 | 0.000000448 | 0.000148861 | 1 | 2.54447E−05 | 0.004756515 |
| ILMN_1215167 | 0.019410925 | 0.000101652 | 0.006701969 | 0.0000543 | 0.004539402 | 1 | 7.0054E−05 | 0.008469621 |
| ILMN_3145331 | 0.013353714 | 4.98E−12 | 1.57E−08 | 1.59E−10 | 0.000000461 | 1 | 2.13433E−07 | 0.00036795 |
| ILMN_1252200 | 0.013353714 | 0.00000761 | 0.001035401 | 0.0000334 | 0.003276709 | 1 | 5.74504E−05 | 0.007618639 |
| ILMN_2819380 | 0.013353714 | 0.000036 | 0.003145034 | 0.00012566 | 0.008308344 | 1 | 0.000197175 | 0.015477107 |
| ILMN_2932359 | 0.013353714 | 5.08E−08 | 0.0000227 | 0.0000011 | 0.000288061 | 1 | 1.53585E−05 | 0.003592421 |
| ILMN_2837493 | 0.013353714 | 5.69E−08 | 0.0000248 | 5.02E−08 | 0.0000324 | 1 | 4.9657E−07 | 0.000525315 |
| ILMN_1230137 | 0.013353714 | 2.06E−08 | 0.0000124 | 0.00000121 | 0.000298774 | 1 | 2.88127E−05 | 0.005099409 |
| ILMN_2944666 | 0.013353714 | 5.52E−08 | 0.0000242 | 2.84E−08 | 0.0000217 | 1 | 2.74304E−07 | 0.000411872 |
| ILMN_1217855 | 0.019410925 | 2.84E−08 | 0.0000149 | 0.000000531 | 0.000169129 | 1 | 0.000264806 | 0.018619193 |
| ILMN_2699898 | 0.013353714 | 1.42E−11 | 3.32E−08 | 1.38E−10 | 0.000000429 | 1 | 1.04521E−07 | 0.000299615 |
| ILMN_2516221 | 0.013353714 | 1.11E−08 | 0.00000752 | 0.000000435 | 0.000147963 | 1 | 1.13766E−05 | 0.003078771 |
| ILMN_2652867 | 0.013353714 | 6.33E−10 | 0.000000819 | 3.26E−08 | 0.0000241 | 1 | 4.12145E−06 | 0.001712865 |
| ILMN_1229197 | 0.013353714 | 4.13E−08 | 0.00002 | 1.88E−08 | 0.0000159 | 1 | 9.77116E−07 | 0.000797927 |
| ILMN_2618148 | 0.013353714 | 2.81E−11 | 5.95E−08 | 1.46E−08 | 0.000013 | 1 | 1.18196E−06 | 0.000887369 |
| ILMN_1214841 | 0.024301667 | 0.0000373 | 0.003229091 | 0.000140817 | 0.009031912 | 1 | 0.002173782 | 0.060770595 |
| ILMN_1257496 | 0.013353714 | 8.57E−08 | 0.0000344 | 0.00000185 | 0.000412402 | 1 | 1.47177E−05 | 0.003531258 |
| ILMN_2421246 | 0.013353714 | 1.42E−09 | 0.00000136 | 4.63E−08 | 0.0000308 | 1 | 5.89742E−06 | 0.002111595 |
| ILMN_2729252 | 0.013353714 | 1.38E−09 | 0.00000136 | 2.06E−08 | 0.0000171 | 1 | 1.2393E−06 | 0.000901339 |
| ILMN_2484932 | 0.013353714 | 6.33E−10 | 0.000000819 | 5.47E−08 | 0.0000344 | 1 | 8.06586E−07 | 0.000708454 |
| ILMN_1221819 | 0.013353714 | 7.12E−09 | 0.00000514 | 2.33E−08 | 0.0000187 | 1 | 9.56517E−06 | 0.002748564 |
| ILMN_2981167 | 0.013353714 | 2.31E−08 | 0.000013 | 1.07E−08 | 0.0000115 | 1 | 3.88359E−07 | 0.000488567 |
| ILMN_2585233 | 0.013353714 | 8.22E−10 | 0.000000934 | 8.63E−08 | 0.0000485 | 1 | 1.41024E−05 | 0.003473151 |
| ILMN_2697415 | 0.013353714 | 5.84E−10 | 0.000000799 | 3.71E−08 | 0.0000262 | 1 | 5.64085E−06 | 0.002083846 |
| ILMN_1230345 | 0.013353714 | 3.22E−09 | 0.00000253 | 1.09E−08 | 0.0000115 | 1 | 1.57497E−07 | 0.000345407 |
| ILMN_2811263 | 0.019410925 | 0.0000175 | 0.001842826 | 0.000153672 | 0.009469751 | 1 | 0.000176289 | 0.014446733 |
| ILMN_2560567 | 0.013353714 | 0.0000009 | 0.00020543 | 0.0000211 | 0.002464033 | 1 | 9.21443E−05 | 0.009952034 |
| ILMN_2820893 | 0.013353714 | 1.27E−13 | 8.87E−10 | 8.13E−12 | 7.57E−08 | 1 | 5.59305E−08 | 0.000249449 |
| ILMN_1246609 | 0.013353714 | 3.02E−11 | 6.12E−08 | 2.46E−09 | 0.00000382 | 1 | 3.34766E−07 | 0.00044521 |
| ILMN_2660551 | 0.013353714 | 0.000000943 | 0.000212156 | 0.00000642 | 0.001075031 | 1 | 0.000130304 | 0.012203733 |
| ILMN_2507761 | 0.013353714 | 0.0000307 | 0.002787143 | 0.0000826 | 0.006091257 | 1 | 9.21504E−05 | 0.009952034 |
| ILMN_2753697 | 0.013353714 | 2.97E−13 | 1.73E−09 | 1.04E−10 | 0.000000395 | 1 | 1.92936E−07 | 0.000345407 |
| ILMN_2601453 | 0.013353714 | 3.23E−11 | 6.26E−08 | 9.08E−10 | 0.00000192 | 1 | 9.77016E−07 | 0.000797927 |
| ILMN_1234565 | 0.013353714 | 0.000000107 | 0.0000412 | 0.000000749 | 0.000219513 | 1 | 8.39227E−06 | 0.002520227 |
| ILMN_2459899 | 0.013353714 | 2.09E−09 | 0.00000174 | 0.000000206 | 0.0000903 | 1 | 3.93982E−06 | 0.001667154 |
| ILMN_2761720 | 0.013353714 | 1.19E−09 | 0.00000123 | 3.43E−08 | 0.0000245 | 1 | 9.77016E−07 | 0.000797927 |
| ILMN_1213954 | 0.013353714 | 0.000000106 | 0.000041 | 0.000000355 | 0.000132883 | 1 | 1.49642E−06 | 0.00102442 |
| ILMN_2632971 | 0.013353714 | 1.78E−09 | 0.00000151 | 2.34E−10 | 0.000000605 | 1 | 6.36427E−09 | 0.000122224 |
| ILMN_2860649 | 0.013353714 | 0.00000014 | 0.0000492 | 0.000000101 | 0.0000545 | 1 | 1.72245E−06 | 0.001083444 |
| ILMN_2722784 | 0.013353714 | 4.91E−08 | 0.0000224 | 0.00000016 | 0.0000754 | 1 | 2.06467E−05 | 0.004309944 |
| ILMN_2667829 | 0.013353714 | 2.77E−11 | 5.95E−08 | 1.89E−10 | 0.000000518 | 1 | 8.49731E−08 | 0.000282517 |
| ILMN_1235499 | 0.013353714 | 0.000000445 | 0.000121754 | 0.00000119 | 0.00029799 | 1 | 2.24533E−05 | 0.004403839 |
| ILMN_2694955 | 0.013353714 | 0.00000158 | 0.000316358 | 0.00000706 | 0.001150107 | 1 | 9.95836E−05 | 0.010463471 |
| ILMN_2491445 | 0.013353714 | 2.23E−08 | 0.0000126 | 0.000000429 | 0.000146987 | 1 | 4.70154E−05 | 0.006881837 |
| ILMN_2661185 | 0.013353714 | 0.00000016 | 0.0000542 | 0.0000016 | 0.000369569 | 1 | 1.81978E−05 | 0.003995524 |
| ILMN_2454823 | 0.013353714 | 0.0000166 | 0.001786344 | 0.00013436 | 0.00874694 | 1 | 0.00057968 | 0.029683566 |
| ILMN_2947526 | 0.013353714 | 0.00000016 | 0.0000542 | 0.000000757 | 0.000219513 | 1 | 7.35697E−06 | 0.002329557 |
| ILMN_2705407 | 0.019410925 | 0.000113957 | 0.007314851 | 0.000246029 | 0.012809759 | 1 | 0.000329201 | 0.021282383 |
| ILMN_1227570 | 0.013353714 | 0.000000136 | 0.0000491 | 0.00000102 | 0.000272691 | 1 | 5.30373E−05 | 0.007480988 |
| ILMN_2475156 | 0.019410925 | 0.000363666 | 0.016386806 | 0.000634885 | 0.023714186 | 1 | 0.001104095 | 0.042298211 |
| ILMN_1214650 | 0.013353714 | 0.0000372 | 0.003229091 | 0.0000222 | 0.00254822 | 1 | 3.68613E−05 | 0.005999236 |
| ILMN_2606825 | 0.013353714 | 0.000000113 | 0.0000431 | 0.000000318 | 0.000121491 | 1 | 7.35697E−06 | 0.002329557 |
| ILMN_1224032 | 0.013353714 | 1.24E−08 | 0.00000822 | 6.12E−08 | 0.0000375 | 1 | 3.76554E−06 | 0.001622913 |
| ILMN_2436183 | 0.013353714 | 8.65E−08 | 0.0000344 | 0.00000254 | 0.000523539 | 1 | 4.7012E−05 | 0.006881837 |
| ILMN_2419490 | 0.013353714 | 7.56E−11 | 0.000000135 | 1.53E−08 | 0.0000132 | 1 | 1.56849E−06 | 0.001042981 |
| ILMN_2432110 | 0.013353714 | 0.000000132 | 0.0000482 | 9.52E−08 | 0.0000521 | 1 | 1.12718E−06 | 0.000874451 |
| ILMN_2443624 | 0.013353714 | 0.000000253 | 0.0000755 | 0.00000244 | 0.000512485 | 1 | 2.24533E−05 | 0.004409839 |
| ILMN_1217913 | 0.013353714 | 2.34E−08 | 0.000013 | 8.68E−08 | 0.0000485 | 1 | 1.02476E−06 | 0.000822404 |
| ILMN_1215649 | 0.013353714 | 0.0000672 | 0.004933586 | 0.000413435 | 0.017763501 | 1 | 0.000393771 | 0.023711359 |
| ILMN_1217629 | 0.013353714 | 2.19E−08 | 0.0000126 | 1.49E−08 | 0.0000131 | 1 | 4.51028E−06 | 0.001809827 |
| ILMN_1215862 | 0.028815909 | 0.019576508 | 0.177396072 | 0.00069086 | 0.02516235 | 0 | 0.001301571 | 0.046179135 |
| ILMN_2721399 | 0.013353714 | 2.77E−10 | 0.000000445 | 4.69E−09 | 0.00000642 | 1 | 1.8046E−06 | 0.001090998 |
| ILMN_1248340 | 0.013353714 | 0.00000275 | 0.000486333 | 0.0000158 | 0.00199766 | 1 | 2.65234E−05 | 0.004803829 |
| ILMN_2618176 | 0.013353714 | 5.43E−10 | 0.000000765 | 2.48E−08 | 0.0000192 | 1 | 7.35697E−06 | 0.002329557 |
| ILMN_1218717 | 0.013353714 | 0.00000442 | 0.000708724 | 0.0000267 | 0.002856495 | 1 | 7.57938E−05 | 0.008797941 |
| ILMN_1247893 | 0.013353714 | 0.00000467 | 0.000729822 | 0.0000243 | 0.002709534 | 1 | 0.000120719 | 0.011491043 |
| ILMN_1224473 | 0.024301667 | 0.000153758 | 0.008953955 | 0.001472162 | 0.0392018 | 1 | 0.00305523 | 0.073418572 |
| ILMN_2419494 | 0.013353714 | 7.66E−10 | 0.000000915 | 4.99E−08 | 0.0000324 | 1 | 5.39498E−06 | 0.002025163 |
| ILMN_2612125 | 0.013353714 | 4.41E−08 | 0.0000212 | 0.00000152 | 0.000360463 | 1 | 9.15833E−06 | 0.002681086 |
| ILMN_2606162 | 0.013353714 | 0.000000478 | 0.000127055 | 0.00000832 | 0.001279253 | 1 | 0.000600122 | 0.030462238 |
| ILMN_1217406 | 0.013353714 | 4.77E−08 | 0.0000223 | 0.000000286 | 0.000110081 | 1 | 1.13766E−05 | 0.003078771 |
| ILMN_2568571 | 0.013353714 | 0.000000537 | 0.000139712 | 0.0000201 | 0.002389566 | 1 | 6.22097E−05 | 0.007890127 |
| ILMN_2818964 | 0.013353714 | 0.0000386 | 0.003287434 | 0.000041 | 0.003797436 | 1 | 3.39668E−05 | 0.00570777 |
| ILMN_2431390 | 0.013353714 | 1.7E−09 | 0.00000149 | 7.75E−08 | 0.0000451 | 1 | 1.23973E−05 | 0.003224592 |
| ILMN_2456911 | 0.013353714 | 0.00000163 | 0.000324593 | 0.0000123 | 0.001649893 | 1 | 0.000540727 | 0.028504193 |
| ILMN_2981169 | 0.013353714 | 0.0000117 | 0.001399848 | 0.00000211 | 0.00045645 | 1 | 3.43889E−06 | 0.001569314 |
| ILMN_1249366 | 0.013353714 | 1.59E−09 | 0.00000145 | 0.000000155 | 0.0000735 | 1 | 5.64035E−06 | 0.002083846 |
| ILMN_1246194 | 0.013353714 | 0.000898696 | 0.029171279 | 0.000236413 | 0.012496999 | 1 | 0.00022037 | 0.016464793 |
| ILMN_1236507 | 0.013353714 | 0.0000665 | 0.00488934 | 0.000431891 | 0.018308965 | 1 | 0.000486846 | 0.026691654 |
| ILMN_1254692 | 0.013353714 | 0.00000049 | 0.000129541 | 0.000000808 | 0.000227616 | 1 | 3.59866E−06 | 0.001580255 |
| ILMN_3156208 | 0.013353714 | 2.38E−08 | 0.000013 | 0.000000386 | 0.00013797 | 1 | 6.44447E−06 | 0.002189569 |
| ILMN_2734391 | 0.013353714 | 5.98E−09 | 0.00000442 | 2.14E−09 | 0.00000357 | 1 | 3.18544E−07 | 0.00044521 |
| ILMN_1224091 | 0.024301667 | 0.000840483 | 0.02804442 | 0.000952078 | 0.030395331 | 1 | 0.001531279 | 0.050161589 |
| ILMN_2456216 | 0.024301667 | 0.0000367 | 0.003197871 | 0.000314936 | 0.015019787 | 1 | 0.000903743 | 0.038000482 |
| ILMN_2627441 | 0.013353714 | 0.00000119 | 0.000254149 | 0.00000113 | 0.000290416 | 1 | 4.31186E−06 | 0.001776143 |
| ILMN_2891245 | 0.039534447 | 0.000501839 | 0.020403653 | 0.001206544 | 0.035013094 | 1 | 0.001977573 | 0.05767797 |
| ILMN_1232804 | 0.013353714 | 0.000000319 | 0.0000928 | 0.000001 | 0.000269028 | 1 | 6.47271E−05 | 0.008034278 |
| ILMN_1232537 | 0.013353714 | 0.000000413 | 0.000115003 | 0.00000371 | 0.000722503 | 1 | 7.28705E−05 | 0.008652811 |
| ILMN_2444432 | 0.013353714 | 0.00000104 | 0.00022691 | 0.00000112 | 0.000290018 | 1 | 7.57938E−05 | 0.008797941 |
| ILMN_2756046 | 0.013353714 | 0.0000389 | 0.003300557 | 0.000000133 | 0.000068 | 1 | 2.99936E−06 | 0.00146959 |
| ILMN_1214163 | 0.013353714 | 6.66E−08 | 0.0000279 | 0.000000461 | 0.000152045 | 1 | 4.16456E−05 | 0.006397623 |
| ILMN_2613832 | 0.013353714 | 0.00000018 | 0.0000594 | 1.27E−08 | 0.0000119 | 1 | 1.49656E−06 | 0.00102442 |
| ILMN_2506039 | 0.013353714 | 0.000000661 | 0.000160197 | 0.000000404 | 0.00013924 | 1 | 7.03988E−06 | 0.002307643 |
| ILMN_1219179 | 0.013353714 | 0.00000165 | 0.00032655 | 0.00000379 | 0.000732759 | 1 | 8.39227E−06 | 0.002520227 |
| ILMN_1248898 | 0.013353714 | 0.00000523 | 0.000783227 | 0.0000278 | 0.002931426 | 1 | 9.5799E−05 | 0.010180718 |
| ILMN_2651575 | 0.013353714 | 0.000000563 | 0.000143319 | 0.00000707 | 0.001150107 | 1 | 1.1878E−05 | 0.003141395 |
| ILMN_2708580 | 0.013353714 | 0.000000133 | 0.0000482 | 0.000000241 | 0.000101903 | 1 | 3.76554E−06 | 0.001622913 |
| ILMN_1236256 | 0.019410925 | 0.000121164 | 0.007605138 | 0.00023881 | 0.012588785 | 1 | 0.00022037 | 0.015464793 |
| ILMN_2525855 | 0.013353714 | 0.0000124 | 0.001448071 | 0.0000814 | 0.006023296 | 1 | 9.5799E−05 | 0.010180718 |
| ILMN_2526938 | 0.013353714 | 0.0000881 | 0.006063068 | 0.000165402 | 0.009948464 | 1 | 0.000393771 | 0.023711359 |
| ILMN_1213664 | 0.019410925 | 0.00000457 | 0.000720402 | 0.0000219 | 0.002525607 | 1 | 0.000157497 | 0.013500967 |
| ILMN_1255743 | 0.013353714 | 0.0000121 | 0.001432469 | 0.0000346 | 0.003367292 | 1 | 0.00011618 | 0.011242898 |
| ILMN_2646625 | 0.013353714 | 4.75E−08 | 0.0000223 | 0.000000043 | 0.0000294 | 1 | 8.0667E−07 | 0.000708454 |
| ILMN_2843019 | 0.013353714 | 0.000000572 | 0.00014393 | 0.00000446 | 0.000810182 | 1 | 3.00244E−05 | 0.00527375 |
| ILMN_3047389 | 0.068774915 | 0.004390712 | 0.075470625 | 0.003544179 | 0.067235737 | 0 | 0.003774648 | 0.082954936 |
| ILMN_1257639 | 0.013353714 | 0.0000254 | 0.002457811 | 0.000140358 | 0.009023792 | 1 | 0.000157487 | 0.013500967 |
| ILMN_1239770 | 0.013353714 | 2.15E−08 | 0.0000125 | 6.23E−08 | 0.0000377 | 1 | 1.56849E−06 | 0.001042981 |
| ILMN_2669841 | 0.013353714 | 0.00000028 | 0.0000823 | 0.000000121 | 0.0000627 | 1 | 1.29931E−06 | 0.000930443 |
| ILMN_2797061 | 0.013353714 | 3.85E−08 | 0.0000189 | 1.24E−08 | 0.0000119 | 1 | 1.18196E−06 | 0.000887369 |
| ILMN_2784580 | 0.013353714 | 0.00000294 | 0.000516117 | 0.0000297 | 0.003067006 | 1 | 0.000763113 | 0.034824155 |
| ILMN_3006219 | 0.013353714 | 0.0000042 | 0.000676604 | 0.0000232 | 0.002627969 | 1 | 0.000157497 | 0.013500967 |
| ILMN_2467190 | 0.013353714 | 0.00000128 | 0.000266467 | 0.0000012 | 0.00029799 | 1 | 2.76434E−05 | 0.004930329 |
| ILMN_2536349 | 0.013353714 | 6.12E−08 | 0.0000262 | 0.000000494 | 0.00016183 | 1 | 4.16456E−05 | 0.006397623 |
| ILMN_2432550 | 0.013353714 | 0.000000208 | 0.0000667 | 0.000000394 | 0.00013797 | 1 | 6.16514E−06 | 0.002125696 |
| ILMN_2844996 | 0.013353714 | 0.00000474 | 0.000733297 | 0.0000112 | 0.001548243 | 1 | 0.000140602 | 0.012732723 |
| ILMN_1247281 | 0.013353714 | 0.000000303 | 0.0000888 | 0.000000759 | 0.000219513 | 1 | 2.73684E−06 | 0.001399911 |
| ILMN_1227814 | 0.059256725 | 0.000325514 | 0.015151707 | 0.001917313 | 0.04584872 | 0 | 0.004782836 | 0.09409752 |
| ILMN_1249021 | 0.013353714 | 2.39E−08 | 0.000013 | 0.000000271 | 0.000109783 | 1 | 7.35697E−06 | 0.002329557 |
| ILMN_2427592 | 0.013353714 | 3.87E−08 | 0.0000189 | 0.000000074 | 0.0000436 | 1 | 8.87963E−07 | 0.000765408 |
| ILMN_1259075 | 0.013353714 | 6.14E−08 | 0.0000262 | 0.000000253 | 0.000106116 | 1 | 3.43889E−06 | 0.001569314 |
| ILMN_1343049 | 0.019410925 | 0.0000486 | 0.003863238 | 0.0000962 | 0.006777584 | 1 | 0.000111795 | 0.010979022 |
| ILMN_3026397 | 0.061967633 | 0.000796299 | 0.027301551 | 0.003501969 | 0.066778429 | 0 | 0.005373402 | 0.100089876 |
| ILMN_2631610 | 0.013353714 | 0.00000196 | 0.00037186 | 0.000000731 | 0.000218104 | 1 | 2.99936E−06 | 0.00146959 |
| ILMN_1227434 | 0.013353714 | 0.00000383 | 0.000632356 | 0.00000744 | 0.001193798 | 1 | 7.57886E−05 | 0.008797941 |
| ILMN_1232600 | 0.013353714 | 7.88E−08 | 0.0000325 | 0.000000526 | 0.000168884 | 1 | 8.0326E−06 | 0.002476116 |
| ILMN_2656854 | 0.013353714 | 0.0000106 | 0.001291118 | 0.000015 | 0.001927918 | 1 | 0.000763113 | 0.034824155 |
| ILMN_1258652 | 0.013353714 | 0.0000026 | 0.000464931 | 0.00000949 | 0.00136995 | 1 | 2.24533E−05 | 0.004409839 |
| ILMN_1245754 | 0.013353714 | 0.0000661 | 0.004874096 | 0.00015814 | 0.009685436 | 1 | 0.00042277 | 0.024660011 |
| ILMN_2780247 | 0.024301667 | 0.0000477 | 0.003816686 | 0.000069 | 0.005349701 | 1 | 0.000665544 | 0.032069437 |
| ILMN_2663930 | 0.013353714 | 0.00000138 | 0.000280532 | 0.00000835 | 0.001279253 | 1 | 0.000111802 | 0.010979022 |
| ILMN_3023230 | 0.013353714 | 0.00000543 | 0.000807251 | 0.0000199 | 0.002370654 | 1 | 0.00011618 | 0.011242898 |
| ILMN_2764727 | 0.013353714 | 1.37E−08 | 0.00000883 | 0.00000044 | 0.000148085 | 1 | 2.65234E−05 | 0.004803829 |
| ILMN_2539295 | 0.019410925 | 0.00000448 | 0.000708724 | 0.000044 | 0.003965776 | 1 | 0.001797898 | 0.054876565 |
| ILMN_2448404 | 0.039534447 | 0.001330158 | 0.03704155 | 0.001691073 | 0.042433629 | 1 | 0.002787111 | 0.070087328 |
| ILMN_1241915 | 0.013353714 | 0.00000308 | 0.00053349 | 0.00000333 | 0.000657593 | 1 | 2.24515E−05 | 0.004409839 |
| ILMN_2605819 | 0.019410925 | 0.00000579 | 0.000845189 | 0.0000358 | 0.003445285 | 1 | 0.000353739 | 0.02219068 |
| ILMN_1253699 | 0.013353714 | 0.00000247 | 0.000456032 | 0.0000131 | 0.001740513 | 1 | 3.12891E−05 | 0.005434371 |
| ILMN_1244853 | 0.013353714 | 0.00000528 | 0.000787896 | 0.000019 | 0.002312072 | 1 | 0.00022037 | 0.016464793 |
| ILMN_2589871 | 0.013353714 | 0.00000485 | 0.000741671 | 0.00000392 | 0.000746457 | 1 | 2.34111E−05 | 0.00450296 |
| ILMN_2997406 | 0.019410925 | 0.0000259 | 0.002476668 | 0.0000788 | 0.005900268 | 1 | 0.001686537 | 0.053258626 |
| ILMN_2860645 | 0.019410925 | 0.000234052 | 0.012078075 | 0.000114517 | 0.007736431 | 1 | 0.000169803 | 0.014189966 |
| ILMN_1249547 | 0.013353714 | 0.0000159 | 0.001744691 | 0.0000524 | 0.004443054 | 1 | 0.000163541 | 0.013740705 |
| ILMN_1236539 | 0.013353714 | 0.00000177 | 0.000345252 | 0.0000425 | 0.00387062 | 1 | 0.000329201 | 0.021282383 |
| ILMN_2425990 | 0.013353714 | 0.0000176 | 0.001843579 | 0.0000196 | 0.002353419 | 1 | 5.89742E−06 | 0.002111595 |
| ILMN_2553041 | 0.042916604 | 0.000119156 | 0.007546078 | 0.000917336 | 0.029755562 | 1 | 0.000873828 | 0.037145252 |
| ILMN_1224945 | 0.013353714 | 0.00000824 | 0.001096103 | 0.0000241 | 0.002707128 | 1 | 1.89821E−05 | 0.004090548 |
| ILMN_1218525 | 0.013353714 | 0.0000071 | 0.00098878 | 0.00000253 | 0.000522839 | 1 | 5.52021E−05 | 0.007602042 |
| ILMN_1243621 | 0.013353714 | 0.000146472 | 0.008715941 | 0.000674016 | 0.024761981 | 1 | 0.00057968 | 0.029683566 |
| ILMN_1228608 | 0.056620496 | 0.001625443 | 0.042197151 | 0.00320971 | 0.063172249 | 0 | 0.005070533 | 0.096927353 |
| ILMN_1229746 | 0.013353714 | 0.00000063 | 0.000155261 | 0.00000018 | 0.0000829 | 1 | 1.89087E−06 | 0.001114107 |
| ILMN_2915951 | 0.013353714 | 0.0000242 | 0.0023517 | 0.0000549 | 0.004568149 | 1 | 5.97852E−05 | 0.007773248 |
| ILMN_1249864 | 0.013353714 | 0.0000339 | 0.003031411 | 0.0000306 | 0.003131644 | 1 | 0.000111802 | 0.010979022 |
| ILMN_2995934 | 0.013353714 | 0.00000251 | 0.000462094 | 0.00000828 | 0.001279253 | 1 | 3.99889E−05 | 0.006183935 |
| ILMN_2456391 | 0.013353714 | 0.0000215 | 0.002169982 | 0.000104677 | 0.007215102 | 1 | 0.001068077 | 0.041672924 |
| ILMN_1239469 | 0.013353714 | 0.00000385 | 0.000632356 | 0.0000028 | 0.000567027 | 1 | 9.56597E−06 | 0.002748564 |
| ILMN_1259463 | 0.013353714 | 0.000000115 | 0.0000431 | 0.000000851 | 0.00023576 | 1 | 2.27642E−06 | 0.00127663 |
| ILMN_3142803 | 0.051552328 | 0.005161613 | 0.083438025 | 0.002307777 | 0.05128928 | 0 | 0.003346676 | 0.077347433 |
| ILMN_2594768 | 0.024301667 | 0.0000101 | 0.00124325 | 0.000186966 | 0.010823311 | 1 | 0.000470028 | 0.02607676 |
| ILMN_2677207 | 0.070737293 | 0.001281123 | 0.036250713 | 0.002803425 | 0.058355577 | 0 | 0.00675613 | 0.111794383 |
| ILMN_1226048 | 0.013353714 | 0.000000496 | 0.000130437 | 0.000000584 | 0.000181161 | 1 | 6.16514E−06 | 0.002125696 |
| ILMN_1241598 | 0.013353714 | 0.0000304 | 0.002783394 | 0.000043 | 0.003909 | 1 | 0.000135352 | 0.012427306 |
| ILMN_1258272 | 0.028815909 | 0.000194457 | 0.010537108 | 0.000331984 | 0.01550964 | 1 | 0.001033149 | 0.040997434 |
| ILMN_1238847 | 0.013353714 | 0.00000182 | 0.00035008 | 0.00000947 | 0.00136995 | 1 | 0.000353739 | 0.02219068 |
| ILMN_1254218 | 0.013353714 | 0.0000288 | 0.002676342 | 0.00013436 | 0.00874694 | 1 | 7.28705E−05 | 0.008652811 |
| ILMN_2879858 | 0.013353714 | 9.55E−12 | 2.47E−08 | 4.3E−12 | 0.00000005 | 1 | 1.05033E−08 | 0.000122224 |
| ILMN_1231490 | 0.013353714 | 0.0000613 | 0.004588271 | 0.000093 | 0.006607866 | 1 | 9.5799E−05 | 0.010180718 |
| ILMN_1232707 | 0.013353714 | 0.00000184 | 0.000353169 | 0.000000273 | 0.000109783 | 1 | 8.0326E−06 | 0.002476116 |
| ILMN_1240592 | 0.117507061 | 0.002418468 | 0.054121371 | 0.008048713 | 0.105414587 | 0 | 0.019643191 | 0.192333843 |
| ILMN_3142602 | 0.013353714 | 0.000000128 | 0.0000472 | 0.000000285 | 0.000110081 | 1 | 1.72245E−06 | 0.001083444 |
| ILMN_3031781 | 0.024301667 | 0.000174461 | 0.009713688 | 0.000378387 | 0.0167608 | 1 | 0.000665544 | 0.032069437 |
| ILMN_2568028 | 0.013353714 | 0.0000163 | 0.001773716 | 0.000108979 | 0.007416137 | 1 | 0.000151664 | 0.013244851 |
| ILMN_1239776 | 0.019410925 | 0.0000766 | 0.005471813 | 0.000378448 | 0.0167608 | 1 | 0.000341264 | 0.021819779 |
| ILMN_1253691 | 0.013353714 | 0.000000376 | 0.000106645 | 0.000000357 | 0.000132883 | 1 | 4.50187E−07 | 0.000525315 |
| ILMN_1239238 | 0.013353714 | 0.00000445 | 0.000708724 | 0.000000927 | 0.000252346 | 1 | 5.30373E−05 | 0.007480988 |
| ILMN_2663230 | 0.019410925 | 0.000000228 | 0.0000712 | 0.00000754 | 0.001205762 | 1 | 0.00010758 | 0.010838827 |
| ILMN_1255869 | 0.013353714 | 0.0000558 | 0.004277499 | 0.0000935 | 0.006622679 | 1 | 0.000135361 | 0.012427306 |
| ILMN_2847773 | 0.013353714 | 0.00000407 | 0.000663099 | 0.00000411 | 0.000768531 | 1 | 3.39668E−05 | 0.00570777 |
| ILMN_2945940 | 0.013353714 | 0.0000964 | 0.006494076 | 0.0000445 | 0.003979315 | 1 | 0.000220357 | 0.016464793 |
| ILMN_2426853 | 0.032842408 | 0.001518945 | 0.04027676 | 0.000354708 | 0.016129651 | 1 | 0.001389344 | 0.047728966 |
| ILMN_2639972 | 0.013353714 | 0.0000018 | 0.000347178 | 0.00000665 | 0.001105293 | 1 | 0.000146035 | 0.012922953 |
| ILMN_2693461 | 0.013353714 | 0.00000811 | 0.001084428 | 0.0000104 | 0.001467743 | 1 | 9.95836E−05 | 0.010463471 |
| ILMN_1258988 | 0.061967633 | 0.000446873 | 0.018841131 | 0.001755338 | 0.043416704 | 0 | 0.012677869 | 0.153956896 |
| ILMN_2509988 | 0.013353714 | 0.000373422 | 0.016697112 | 0.000493488 | 0.020167155 | 1 | 0.000317538 | 0.020788254 |
| ILMN_1230458 | 0.032842408 | 0.003998446 | 0.07166564 | 0.001978755 | 0.046801373 | 0 | 0.001301631 | 0.046179135 |
| ILMN_2727235 | 0.028815909 | 0.000575054 | 0.022103239 | 0.001101727 | 0.033170811 | 1 | 0.004006777 | 0.085591297 |
| ILMN_2637714 | 0.013353714 | 0.000000138 | 0.0000492 | 7.38E−08 | 0.0000436 | 1 | 1.92936E−07 | 0.000345407 |
| ILMN_2595918 | 0.019410925 | 0.00000021 | 0.0000667 | 0.00000695 | 0.001138958 | 1 | 0.000183009 | 0.014712435 |
| ILMN_1231573 | 0.013353714 | 0.000000905 | 0.000205533 | 0.00000222 | 0.000472255 | 1 | 3.83949E−05 | 0.006099544 |
| ILMN_2459155 | 0.013353714 | 0.000171618 | 0.009624487 | 0.000316707 | 0.015073371 | 1 | 0.000470028 | 0.02607676 |
| ILMN_1243910 | 0.013353714 | 0.000162342 | 0.009237839 | 0.000354016 | 0.016129651 | 1 | 0.000712779 | 0.033344447 |
| ILMN_1222471 | 0.013353714 | 9.07E−09 | 0.0000064 | 9.8E−09 | 0.0000111 | 1 | 5.74828E−07 | 0.000569287 |
| ILMN_2469190 | 0.024301667 | 0.00017922 | 0.009895781 | 0.000539236 | 0.021235038 | 1 | 0.000212355 | 0.016047225 |
| ILMN_2696492 | 0.019410925 | 0.0000941 | 0.00638343 | 0.000151782 | 0.009420008 | 1 | 0.000504223 | 0.027322568 |
| ILMN_2996904 | 0.013353714 | 0.00000351 | 0.000592532 | 0.00000887 | 0.001330104 | 1 | 2.88127E−05 | 0.005099409 |
| ILMN_1233449 | 0.013353714 | 0.00000334 | 0.000570075 | 0.00000669 | 0.00110836 | 1 | 0.000212368 | 0.016047225 |
| ILMN_1251193 | 0.013353714 | 0.000321826 | 0.015055331 | 0.000250502 | 0.012926945 | 1 | 0.000379979 | 0.02324165 |
| ILMN_2758878 | 0.036357572 | 0.000437488 | 0.018614041 | 0.000914744 | 0.029733662 | 0 | 0.003149577 | 0.074838758 |
| ILMN_3160137 | 0.078761363 | 0.000824708 | 0.027696748 | 0.002110097 | 0.048647455 | 0 | 0.012022966 | 0.149554514 |
| ILMN_1218051 | 0.028815909 | 0.0000255 | 0.002460142 | 0.0000551 | 0.00457056 | 1 | 0.000643 | 0.031573087 |
| ILMN_1230726 | 0.024301667 | 0.000222228 | 0.011677508 | 0.000818902 | 0.027904422 | 1 | 0.001581552 | 0.051264971 |
| ILMN_2879614 | 0.019410925 | 0.0000132 | 0.001509679 | 0.0000283 | 0.002963331 | 1 | 0.000255296 | 0.018170157 |
| ILMN_2534207 | 0.013353714 | 3.59E−08 | 0.000018 | 8.2E−09 | 0.0000103 | 1 | 7.68761E−07 | 0.000701637 |
| ILMN_2659739 | 0.013353714 | 0.0000625 | 0.004670026 | 0.0000102 | 0.001439966 | 1 | 7.88278E−05 | 0.009015226 |
| ILMN_2958159 | 0.013353714 | 0.00000265 | 0.000471163 | 0.0000399 | 0.003729707 | 1 | 0.000329201 | 0.021282383 |
| ILMN_1257704 | 0.019410925 | 0.0000499 | 0.003931786 | 0.000196678 | 0.011191632 | 1 | 0.000197175 | 0.015477107 |
| ILMN_2477213 | 0.013353714 | 0.000000265 | 0.0000787 | 0.000000757 | 0.000219513 | 1 | 4.12145E−06 | 0.001712865 |
| ILMN_2513870 | 0.013353714 | 0.000000243 | 0.0000731 | 0.00000252 | 0.000522839 | 1 | 0.000163531 | 0.013740705 |
| ILMN_1343061 | 0.27805718 | 0.044430396 | 0.27002241 | 0.050912329 | 0.282020251 | 0 | 0.090139534 | 0.395157894 |
| ILMN_1258340 | 0.013353714 | 0.0000229 | 0.002262156 | 0.000017 | 0.002131671 | 1 | 2.06483E−05 | 0.004309944 |
| ILMN_2777019 | 0.019410925 | 0.000170606 | 0.009614023 | 0.000037 | 0.003539707 | 1 | 0.000157497 | 0.013500967 |
| ILMN_1255907 | 0.013353714 | 0.0000147 | 0.00164902 | 0.0000292 | 0.003023837 | 1 | 4.51564E−05 | 0.006758509 |
| ILMN_2449620 | 0.013353714 | 0.0000477 | 0.003816586 | 0.000190185 | 0.010942585 | 1 | 7.57938E−05 | 0.008797941 |
| ILMN_2477946 | 0.013353714 | 0.00000865 | 0.001137893 | 0.000027 | 0.002873981 | 1 | 0.00040803 | 0.024132893 |
| ILMN_2627179 | 0.013353714 | 9.29E−09 | 0.00000645 | 1.03E−08 | 0.0000115 | 1 | 1.74307E−07 | 0.000345407 |
| ILMN_1214318 | 0.013353714 | 0.0000771 | 0.005486903 | 0.0000845 | 0.006173424 | 1 | 0.000873828 | 0.037145252 |
| ILMN_1218206 | 0.032842408 | 0.00013977 | 0.008482243 | 0.000569012 | 0.022034784 | 1 | 0.002540648 | 0.066400639 |
| ILMN_1233501 | 0.028815909 | 0.000144506 | 0.008645643 | 0.000609547 | 0.023104722 | 1 | 0.000737547 | 0.034262098 |
| ILMN_1241522 | 0.013353714 | 0.000000662 | 0.000160197 | 0.00000201 | 0.000441415 | 1 | 6.16514E−06 | 0.002125696 |
| ILMN_1226239 | 0.036357572 | 0.000195832 | 0.010599276 | 0.000409995 | 0.01769293 | 1 | 0.000540669 | 0.028504193 |
| ILMN_2613422 | 0.019410925 | 0.0000104 | 0.001272684 | 0.000056 | 0.004602895 | 1 | 0.000140602 | 0.012732723 |
| ILMN_2593554 | 0.013353714 | 0.0000358 | 0.003145034 | 0.00000464 | 0.000834646 | 1 | 1.67194E−05 | 0.003796597 |
| ILMN_1229804 | 0.046026289 | 0.000286115 | 0.01388718 | 0.001210751 | 0.035093672 | 0 | 0.008450429 | 0.125387993 |
| ILMN_2607160 | 0.013353714 | 0.00000172 | 0.000335874 | 0.00000121 | 0.000298774 | 1 | 2.76434E−05 | 0.004930329 |
| ILMN_3072437 | 0.046026289 | 0.000890124 | 0.029037446 | 0.001222703 | 0.035284033 | 0 | 0.00210642 | 0.059970456 |
| ILMN_1251170 | 0.013353714 | 0.0000524 | 0.004058867 | 0.000114081 | 0.007718214 | 1 | 7.0054E−05 | 0.008469621 |
| ILMN_1246582 | 0.019410925 | 0.0000207 | 0.002110867 | 0.0000462 | 0.004074421 | 1 | 0.000176289 | 0.014446733 |
| ILMN_1244891 | 0.013353714 | 0.0000285 | 0.002659806 | 0.0000142 | 0.001859837 | 1 | 0.000146035 | 0.012922953 |
| ILMN_2697693 | 0.019410925 | 0.0000147 | 0.00164902 | 0.0000385 | 0.003609572 | 1 | 0.000157497 | 0.013500967 |
| ILMN_1233402 | 0.013353714 | 0.000000169 | 0.0000566 | 0.00000129 | 0.000312387 | 1 | 7.03988E−06 | 0.002307643 |
| ILMN_1258864 | 0.019410925 | 0.000437229 | 0.018614041 | 0.000591655 | 0.022629221 | 1 | 0.000317538 | 0.020788254 |
| ILMN_1234796 | 0.013353714 | 0.0000811 | 0.00570333 | 0.000213294 | 0.011819303 | 1 | 0.000197175 | 0.015477107 |
| ILMN_1236595 | 0.028815909 | 0.0000231 | 0.002276062 | 0.000247222 | 0.012817366 | 1 | 0.001344859 | 0.046961097 |
| ILMN_1241952 | 0.019410925 | 0.000284848 | 0.013869053 | 0.000980227 | 0.030953762 | 1 | 0.00148248 | 0.049612679 |
| ILMN_2657980 | 0.013353714 | 0.0000342 | 0.003043914 | 0.000140887 | 0.009031912 | 1 | 0.00040803 | 0.024132893 |
| ILMN_2745614 | 0.013353714 | 0.0000227 | 0.002256884 | 0.000034 | 0.003321814 | 1 | 0.000264806 | 0.018619193 |
| ILMN_1240415 | 0.013353714 | 0.0000121 | 0.001432469 | 0.0000715 | 0.005501275 | 1 | 0.000163531 | 0.013740705 |
| ILMN_2552490 | 0.013353714 | 0.0000289 | 0.002676832 | 0.000121849 | 0.008102409 | 1 | 0.000317538 | 0.020788254 |
| ILMN_2729447 | 0.013353714 | 0.00000038 | 0.000107295 | 0.00000154 | 0.000360893 | 1 | 1.24004E−05 | 0.003224592 |
| ILMN_1233293 | 0.013353714 | 0.000770337 | 0.026798849 | 0.0000253 | 0.002787107 | 1 | 3.39668E−05 | 0.00570777 |
| ILMN_1236105 | 0.039534447 | 0.00046297 | 0.019205635 | 0.002468619 | 0.053569604 | 0 | 0.003663228 | 0.081662975 |
| ILMN_3009860 | 0.013353714 | 0.00000258 | 0.000464931 | 0.0000101 | 0.001437553 | 1 | 6.47271E−05 | 0.008034278 |
| ILMN_1217929 | 0.024301667 | 0.000301908 | 0.014429727 | 0.000612506 | 0.023197995 | 1 | 0.000763113 | 0.034824155 |
| ILMN_1219896 | 0.019410925 | 0.000172522 | 0.009663511 | 0.000360138 | 0.0162751 | 1 | 0.000169803 | 0.014189966 |
| ILMN_2808485 | 0.024301667 | 0.000895135 | 0.029136956 | 0.000321957 | 0.015214362 | 1 | 0.000903743 | 0.038000482 |
| ILMN_2628271 | 0.019410925 | 0.00000934 | 0.00118696 | 0.00014347 | 0.009094873 | 1 | 0.001741399 | 0.054110078 |
| ILMN_2959272 | 0.013353714 | 0.000127096 | 0.007814972 | 0.0000785 | 0.005894011 | 1 | 5.52021E−05 | 0.007602042 |
| ILMN_1240857 | 0.013353714 | 0.00000712 | 0.00098878 | 0.0000106 | 0.001479487 | 1 | 1.08955E−05 | 0.00300091 |
| ILMN_1248837 | 0.019410925 | 0.0000496 | 0.003917954 | 0.0000556 | 0.004598957 | 1 | 0.00010758 | 0.010838827 |
| ILMN_1213986 | 0.013353714 | 0.0000257 | 0.002474589 | 0.0000184 | 0.002261567 | 1 | 2.24533E−05 | 0.004409839 |
| ILMN_2565089 | 0.013353714 | 0.0000412 | 0.003471427 | 0.000057 | 0.00465062 | 1 | 3.99889E−05 | 0.006183935 |
| ILMN_2756628 | 0.013353714 | 1.76E−09 | 0.00000151 | 6.75E−09 | 0.00000872 | 1 | 3.51784E−07 | 0.000454847 |
| ILMN_2689307 | 0.013353714 | 0.0000723 | 0.00525285 | 0.000235982 | 0.01249632 | 1 | 0.000284828 | 0.019439747 |
| ILMN_2551741 | 0.019410925 | 0.000463127 | 0.019206635 | 0.001042662 | 0.032055996 | 1 | 0.000540698 | 0.028504193 |
| ILMN_1343062 | 0.295273635 | 0.052227493 | 0.293047327 | 0.059487654 | 0.305188121 | 0 | 0.168994973 | 0.521874145 |
| ILMN_2749364 | 0.036357572 | 0.0000498 | 0.003931092 | 0.000245333 | 0.01278781 | 0 | 0.002963349 | 0.072406838 |
| ILMN_2784272 | 0.036357572 | 0.000859398 | 0.028451212 | 0.001629498 | 0.041583456 | 1 | 0.000903743 | 0.038000482 |
| ILMN_1225348 | 0.013353714 | 0.00000714 | 0.000989051 | 0.0000248 | 0.002755904 | 1 | 0.000189968 | 0.015089482 |
| ILMN_2630739 | 0.032842408 | 0.000519149 | 0.020795881 | 0.000419249 | 0.017887063 | 1 | 0.000393771 | 0.023711359 |
| ILMN_2418366 | 0.013353714 | 0.000145382 | 0.008676736 | 0.000268314 | 0.013575239 | 1 | 0.000540727 | 0.028504193 |
| ILMN_2846731 | 0.013353714 | 2.88E−08 | 0.0000149 | 0.000000145 | 0.0000709 | 1 | 2.15328E−05 | 0.004320203 |
| ILMN_2594855 | 0.013353714 | 0.0000466 | 0.003774527 | 0.0000558 | 0.004598957 | 1 | 0.000189956 | 0.015089482 |
| ILMN_1246543 | 0.013353714 | 0.000320008 | 0.015015535 | 0.000452506 | 0.018972559 | 1 | 0.000737547 | 0.034262098 |
| ILMN_2614884 | 0.013353714 | 0.00000151 | 0.000303563 | 0.00000406 | 0.000761994 | 1 | 1.74444E−05 | 0.003903768 |
| ILMN_1250418 | 0.013353714 | 0.000000139 | 0.0000492 | 0.000000276 | 0.000109783 | 1 | 2.27642E−06 | 0.00127663 |
| ILMN_2706514 | 0.019410925 | 0.000100461 | 0.006651733 | 0.000328884 | 0.015447583 | 1 | 0.001581552 | 0.051264971 |
| ILMN_1241924 | 0.013353714 | 0.0000299 | 0.002749923 | 0.000106267 | 0.007284834 | 1 | 0.00027463 | 0.019109041 |
| ILMN_2750265 | 0.028815909 | 0.000407095 | 0.017742578 | 0.000921456 | 0.02980611 | 1 | 0.001915982 | 0.056660247 |
| ILMN_2712895 | 0.013353714 | 0.0000422 | 0.003521702 | 0.000104785 | 0.007215102 | 1 | 0.000176289 | 0.014446733 |
| ILMN_2719139 | 0.019410925 | 0.000234828 | 0.012104699 | 0.000294012 | 0.014330244 | 1 | 0.001581552 | 0.051264971 |
| ILMN_2692986 | 0.028815909 | 0.000159639 | 0.009162023 | 0.000164723 | 0.009931795 | 1 | 0.0007895 | 0.035437666 |
| ILMN_1228333 | 0.028815909 | 0.000105496 | 0.006916233 | 0.000123189 | 0.008156571 | 0 | 0.002173782 | 0.060770595 |
| ILMN_2940446 | 0.032842408 | 0.0000799 | 0.005652909 | 0.000441159 | 0.018633965 | 1 | 0.003449313 | 0.078819425 |
| ILMN_3034381 | 0.024301667 | 0.000114964 | 0.007350619 | 0.000169394 | 0.010108673 | 1 | 0.000246108 | 0.017705716 |
| ILMN_2668387 | 0.013353714 | 0.00000034 | 0.0000984 | 0.000000168 | 0.000078 | 1 | 3.59866E−06 | 0.001580255 |
| ILMN_2592953 | 0.013353714 | 0.000026 | 0.002476668 | 0.0000347 | 0.003370075 | 1 | 0.000228655 | 0.016840488 |
| ILMN_1228867 | 0.013353714 | 0.00000462 | 0.000724818 | 0.0000117 | 0.001595024 | 1 | 0.00042277 | 0.024660011 |
| ILMN_2785454 | 0.106524046 | 0.008895037 | 0.113652841 | 0.010392592 | 0.121665992 | 0 | 0.049191671 | 0.30108149 |
| ILMN_2773900 | 0.019410925 | 0.000000602 | 0.000149919 | 0.00000244 | 0.000512485 | 1 | 3.83949E−05 | 0.006099544 |
| ILMN_2647102 | 0.013353714 | 0.00000884 | 0.001152122 | 0.0000424 | 0.00387062 | 1 | 7.57938E−05 | 0.008797941 |
| ILMN_1256574 | 0.024301667 | 0.0000493 | 0.003911645 | 0.00022291 | 0.012129499 | 1 | 0.00040803 | 0.024132893 |
| ILMN_1243902 | 0.036357572 | 0.000455051 | 0.019030791 | 0.000881288 | 0.029196677 | 1 | 0.00305523 | 0.073418572 |
| ILMN_1343048 | 0.019410925 | 0.000238559 | 0.012189026 | 0.000271705 | 0.013620278 | 1 | 0.000934606 | 0.038635069 |
| ILMN_2878979 | 0.013353714 | 0.000000464 | 0.000126212 | 0.00000373 | 0.000722783 | 1 | 1.74444E−05 | 0.003903768 |
| ILMN_2887619 | 0.231276599 | 0.025251221 | 0.201975034 | 0.032264118 | 0.224678675 | 0 | 0.039320852 | 0.270990186 |
| ILMN_2678724 | 0.013353714 | 0.000216155 | 0.011472502 | 0.000287644 | 0.014123395 | 1 | 0.000379979 | 0.02324165 |
| ILMN_1244135 | 0.059256725 | 0.007037524 | 0.099597335 | 0.005589714 | 0.086699245 | 0 | 0.00450999 | 0.091557737 |
| ILMN_2651297 | 0.013353714 | 0.00000985 | 0.001218839 | 0.00000212 | 0.00045645 | 1 | 1.35117E−05 | 0.003455664 |
| ILMN_2822842 | 0.013353714 | 0.0000173 | 0.001831837 | 0.00000567 | 0.00097714 | 1 | 1.97985E−05 | 0.004208042 |
| ILMN_2665545 | 0.013353714 | 0.0000502 | 0.003943501 | 0.0000406 | 0.003782136 | 1 | 0.000246108 | 0.017705716 |
| ILMN_2703321 | 0.013353714 | 0.0000123 | 0.001443502 | 0.0000441 | 0.003965776 | 1 | 0.000146035 | 0.012922953 |
| ILMN_1259917 | 0.013353714 | 0.000210302 | 0.011263162 | 0.000345083 | 0.015942739 | 1 | 0.000353739 | 0.02219068 |
| ILMN_1237871 | 0.013353714 | 0.000036 | 0.003145034 | 0.0000107 | 0.001483462 | 1 | 4.70154E−05 | 0.006881837 |
| ILMN_1256950 | 0.019410925 | 0.0000169 | 0.001800169 | 0.0000192 | 0.002327044 | 1 | 3.83949E−05 | 0.006099544 |
| ILMN_1228020 | 0.024301667 | 0.0000866 | 0.005995631 | 0.000234419 | 0.012474872 | 1 | 0.000934606 | 0.038635069 |
| ILMN_1227573 | 0.061967633 | 0.002318102 | 0.052579328 | 0.004453929 | 0.076746435 | 0 | 0.002387591 | 0.063870812 |
| ILMN_2726315 | 0.019410925 | 0.00000926 | 0.00118448 | 0.0000286 | 0.002983758 | 1 | 0.000621233 | 0.03109305 |
| ILMN_1227907 | 0.013353714 | 1.41E−08 | 0.00000898 | 0.000000113 | 0.0000599 | 1 | 7.03866E−06 | 0.002307643 |
| ILMN_2881296 | 0.013353714 | 0.00000179 | 0.000347178 | 0.00000451 | 0.000814057 | 1 | 1.29447E−05 | 0.003328934 |
| ILMN_3043245 | 0.013353714 | 0.000000231 | 0.0000712 | 0.000000523 | 0.000168884 | 1 | 3.93982E−06 | 0.001667154 |
| ILMN_2580159 | 0.024301667 | 0.000556709 | 0.021670373 | 0.001253562 | 0.035750241 | 1 | 0.000712779 | 0.033344447 |
| ILMN_1252472 | 0.048451528 | 0.000596303 | 0.022658053 | 0.002340061 | 0.051632853 | 0 | 0.001856078 | 0.055774594 |
| ILMN_2828916 | 0.019410925 | 0.000173432 | 0.009681132 | 0.0000582 | 0.004700168 | 1 | 0.000182997 | 0.014712435 |
| ILMN_1257771 | 0.024301667 | 0.0000793 | 0.005618146 | 0.0000839 | 0.006145701 | 1 | 0.001218998 | 0.044502503 |
| ILMN_3002095 | 0.019410925 | 0.0000149 | 0.001663783 | 0.0000489 | 0.004233399 | 1 | 0.000621233 | 0.03109305 |
| ILMN_2658961 | 0.036357572 | 0.000265914 | 0.013252143 | 0.000649417 | 0.024048059 | 1 | 0.002314175 | 0.062958735 |
| ILMN_2038771 | 0.282520365 | 0.057007789 | 0.305496381 | 0.05139364 | 0.283318913 | 0 | 0.154914896 | 0.50337338 |
| ILMN_2981801 | 0.019410925 | 0.000371616 | 0.016648319 | 0.000201538 | 0.011374568 | 1 | 0.000504223 | 0.027322568 |
| ILMN_1249710 | 0.013353714 | 0.0000376 | 0.003237706 | 0.000154008 | 0.009469751 | 1 | 0.000306263 | 0.02048222 |
| ILMN_2943057 | 0.024301657 | 0.000428331 | 0.018375605 | 0.000443074 | 0.018680938 | 1 | 0.000763113 | 0.034824155 |
| ILMN_1235856 | 0.013353714 | 0.00024238 | 0.012324478 | 0.000221541 | 0.012090825 | 1 | 0.000103509 | 0.010612415 |
| ILMN_1247942 | 0.019410925 | 0.000306942 | 0.014534302 | 0.000519622 | 0.020743454 | 1 | 0.00042277 | 0.024660011 |
| ILMN_1258509 | 0.013353714 | 0.000151735 | 0.008884051 | 0.000102346 | 0.007068104 | 1 | 0.000237231 | 0.017362241 |
| ILMN_2544056 | 0.224859756 | 0.020977231 | 0.184099808 | 0.029221576 | 0.212190751 | 0 | 0.025142431 | 0.218223503 |
| ILMN_2729953 | 0.074974418 | 0.003641212 | 0.067632684 | 0.005035407 | 0.081966426 | 0 | 0.008450429 | 0.125387993 |
| ILMN_1219602 | 0.059256725 | 0.006317929 | 0.093553116 | 0.002151161 | 0.049131535 | 0 | 0.004251793 | 0.088827736 |
| ILMN_2681670 | 0.013353714 | 0.00000824 | 0.001096103 | 0.0000174 | 0.002162819 | 1 | 6.22097E−05 | 0.007890127 |
| ILMN_1234955 | 0.039534447 | 0.000742364 | 0.026039808 | 0.001614985 | 0.04134913 | 1 | 0.002387591 | 0.063870812 |
| ILMN_2582659 | 0.013353714 | 0.0000266 | 0.002516087 | 0.000057 | 0.00465062 | 1 | 0.00010758 | 0.010838827 |
| ILMN_1252514 | 0.028815909 | 0.000151132 | 0.008859861 | 0.000433591 | 0.018347587 | 1 | 0.0007895 | 0.035437666 |
| ILMN_2617433 | 0.054324155 | 0.002870664 | 0.059952728 | 0.002726633 | 0.057221177 | 0 | 0.00553098 | 0.101481885 |
| ILMN_2738972 | 0.013353714 | 0.0000107 | 0.001302986 | 0.00000896 | 0.00133296 | 1 | 2.65234E−05 | 0.004803829 |
| ILMN_2553280 | 0.013353714 | 0.00000649 | 0.000934608 | 0.0000318 | 0.003192608 | 1 | 0.000111802 | 0.010979022 |
| ILMN_1238725 | 0.138695553 | 0.012860546 | 0.140951218 | 0.014592729 | 0.14770671 | 0 | 0.023367993 | 0.210226124 |
| ILMN_2998813 | 0.013353714 | 0.0000207 | 0.002110867 | 0.0000384 | 0.003609572 | 1 | 8.86334E−05 | 0.009822905 |
| ILMN_1236445 | 0.036357572 | 0.000961228 | 0.030645413 | 0.001935598 | 0.046142977 | 1 | 0.00305523 | 0.073418572 |
| ILMN_1220893 | 0.013353714 | 0.00000124 | 0.000260429 | 0.000000812 | 0.000227616 | 1 | 5.3945E−06 | 0.002025163 |
| ILMN_2417398 | 0.076777595 | 0.009191991 | 0.115919703 | 0.004863496 | 0.080501378 | 0 | 0.005531174 | 0.101481885 |
| ILMN_1238893 | 0.019410925 | 0.000124926 | 0.007753235 | 0.000143613 | 0.009094873 | 1 | 0.001141234 | 0.042874093 |
| ILMN_2706550 | 0.024301667 | 0.000143784 | 0.008619217 | 0.000167018 | 0.010005399 | 1 | 0.000816732 | 0.036034504 |
| ILMN_1242235 | 0.013353714 | 0.000000923 | 0.000208593 | 0.00000447 | 0.00081037 | 1 | 7.28705E−05 | 0.008652811 |
| ILMN_2638923 | 0.078761363 | 0.007123051 | 0.100168171 | 0.007948853 | 0.10463667 | 0 | 0.002620495 | 0.067689332 |
| ILMN_1232894 | 0.013353714 | 0.0000217 | 0.002174307 | 0.0000588 | 0.004724126 | 1 | 0.00010758 | 0.010838827 |
| ILMN_1218799 | 0.013353714 | 0.00000545 | 0.00080859 | 0.00000586 | 0.00100296 | 1 | 1.04339E−05 | 0.002925693 |
| ILMN_1257209 | 0.013353714 | 0.0000145 | 0.001637789 | 0.000022 | 0.002530716 | 1 | 9.57926E−05 | 0.010180718 |
| ILMN_2711705 | 0.013353714 | 0.000000199 | 0.0000652 | 0.0000011 | 0.000288061 | 1 | 3.99889E−05 | 0.006183935 |
| ILMN_1236202 | 0.024301667 | 0.000302563 | 0.014429727 | 0.000998638 | 0.031261571 | 1 | 0.002041151 | 0.058756619 |
| ILMN_1233474 | 0.074974418 | 0.001679483 | 0.043166689 | 0.003200353 | 0.063041403 | 0 | 0.005220076 | 0.098611557 |
| ILMN_2762863 | 0.013353714 | 0.000192589 | 0.010448077 | 0.000197935 | 0.011235697 | 1 | 0.0007895 | 0.035437666 |
| ILMN_2644350 | 0.019410925 | 0.0000457 | 0.003725334 | 0.00012026 | 0.008031164 | 1 | 0.002463032 | 0.065251421 |
| ILMN_1255860 | 0.013353714 | 0.000179589 | 0.009904436 | 0.0000691 | 0.005350074 | 1 | 0.000183009 | 0.014712435 |
| ILMN_2636403 | 0.013353714 | 0.000000985 | 0.000217336 | 0.000000614 | 0.00018775 | 1 | 1.08955E−05 | 0.00300091 |
| ILMN_2918002 | 0.013353714 | 0.000573593 | 0.02206532 | 0.000153193 | 0.009469751 | 1 | 0.0007895 | 0.035437660 |
| ILMN_2649068 | 0.090032894 | 0.006628845 | 0.096046173 | 0.005888889 | 0.088967898 | 0 | 0.006383386 | 0.109238048 |
| ILMN_1215939 | 0.019410925 | 0.0000276 | 0.002581184 | 0.000058 | 0.004693744 | 1 | 0.000111795 | 0.010979022 |
| ILMN_2520264 | 0.032842408 | 0.000652141 | 0.023901742 | 0.001090408 | 0.033000801 | 1 | 0.001389407 | 0.047728966 |
| ILMN_1220889 | 0.013353714 | 0.00041846 | 0.018119119 | 0.0000489 | 0.004233399 | 1 | 0.000103509 | 0.010612415 |
| ILMN_2706803 | 0.013353714 | 0.000121041 | 0.007605138 | 0.0000382 | 0.003601497 | 1 | 0.000212368 | 0.016047225 |
| ILMN_2751603 | 0.127562936 | 0.00581702 | 0.089377512 | 0.019672927 | 0.172805787 | 0 | 0.017311316 | 0.180185556 |
| ILMN_1240030 | 0.013353714 | 0.0000187 | 0.001936608 | 0.000099 | 0.006922165 | 1 | 0.000204639 | 0.01592867 |
| ILMN_1242390 | 0.074974418 | 0.002259109 | 0.051954924 | 0.003105507 | 0.061933186 | 0 | 0.002963349 | 0.072406838 |
| ILMN_2510694 | 0.036357572 | 0.000180109 | 0.009909602 | 0.000250403 | 0.012926945 | 1 | 0.002540648 | 0.066400639 |
| ILMN_1258466 | 0.028815909 | 0.0000863 | 0.005995631 | 0.000491484 | 0.020138305 | 1 | 0.001104095 | 0.042298211 |
| ILMN_1252089 | 0.019410925 | 0.000449807 | 0.018930527 | 0.00134792 | 0.037363442 | 1 | 0.001344859 | 0046961097 |
| ILMN_2522571 | 0.013353714 | 0.000171497 | 0.009624487 | 0.0000765 | 0.005770769 | 1 | 1.81978E−05 | 0.003995524 |
| ILMN_2593002 | 0.013353714 | 0.0000472 | 0.003800848 | 0.00000968 | 0.001390556 | 1 | 4.89467E−05 | 0.007053629 |
| ILMN_1248379 | 0.013353714 | 0.0000115 | 0.001387523 | 0.000012 | 0.001629081 | 1 | 1.53585E−05 | 0.003592421 |
| ILMN_1223119 | 0.028815909 | 0.0000582 | 0.004418055 | 0.000128727 | 0.008463041 | 1 | 0.000688785 | 0.032681842 |
| ILMN_3059798 | 0.024301667 | 0.000178164 | 0.009849192 | 0.000280684 | 0.013898949 | 1 | 0.000688785 | 0.032681842 |
| ILMN_2534794 | 0.064412031 | 0.000303005 | 0.014435986 | 0.000928884 | 0.029963118 | 0 | 0.002620495 | 0.067689332 |
| ILMN_2618549 | 0.028815909 | 0.001879355 | 0.046456904 | 0.001945449 | 0.046343295 | 1 | 0.001915982 | 0.056660247 |
| ILMN_2468405 | 0.013353714 | 0.000000209 | 0.0000667 | 0.00000224 | 0.00047443 | 1 | 9.99008E−06 | 0.002818469 |
| ILMN_1258587 | 0.013353714 | 0.00000504 | 0.000761749 | 0.00000641 | 0.001075031 | 1 | 2.76413E−05 | 0.004930329 |
| ILMN_1218732 | 0.013353714 | 0.0000688 | 0.005018833 | 0.000136749 | 0.008839595 | 1 | 0.000183009 | 0.014712435 |
| ILMN_1237980 | 0.013353714 | 0.000147151 | 0.008736505 | 0.000319145 | 0.015112162 | 1 | 0.000486846 | 0.026691654 |
| ILMN_1214874 | 0.013353714 | 0.0000748 | 0.005380504 | 0.000102298 | 0.007068104 | 1 | 0.000844832 | 0.036512914 |
| ILMN_1220418 | 0.013353714 | 0.000150502 | 0.008856418 | 0.0000483 | 0.004205779 | 1 | 5.74463E−05 | 0.007618639 |
| ILMN_2550291 | 0.019410925 | 0.000109438 | 0.007113974 | 0.000121057 | 0.008061295 | 1 | 0.000135361 | 0.012427306 |
| ILMN_1234202 | 0.024301667 | 0.00090293 | 0.029282308 | 0.001346813 | 0.037363102 | 1 | 0.000486846 | 0.026691654 |
| ILMN_2426949 | 0.019410925 | 0.0000768 | 0.005474285 | 0.00020508 | 0.011473406 | 1 | 0.000453754 | 0.02560106 |
| ILMN_2497395 | 0.028815909 | 0.000278037 | 0.013666084 | 0.000762961 | 0.026721993 | 1 | 0.002106509 | 0.059970456 |
| ILMN_2715730 | 0.019410925 | 0.0000115 | 0.00138491 | 0.0000749 | 0.005687727 | 1 | 0.000284828 | 0.019439747 |
| ILMN_1217606 | 0.032842408 | 0.000360722 | 0.016283385 | 0.000242261 | 0.012705581 | 1 | 0.000737547 | 0.034262098 |
| ILMN_1216313 | 0.042916604 | 0.001389435 | 0.037998849 | 0.003408037 | 0.065686921 | 0 | 0.004644684 | 0.093111105 |
| ILMN_1252761 | 0.019410925 | 0.000967613 | 0.030803623 | 0.000483066 | 0.019916078 | 1 | 0.000486846 | 0.026691654 |
| ILMN_2710312 | 0.013353714 | 0.00000957 | 0.001210856 | 0.0000519 | 0.004442214 | 1 | 0.000470003 | 0.02607676 |
| ILMN_1232668 | 0.013353714 | 0.000265521 | 0.013246761 | 0.000209487 | 0.011649906 | 1 | 0.000212368 | 0.016047225 |
| ILMN_2593774 | 0.013353714 | 0.00000489 | 0.000743473 | 0.00000986 | 0.001403781 | 1 | 6.47271E−05 | 0.008034278 |
| ILMN_2559724 | 0.10074305 | 0.005102656 | 0.082814962 | 0.006787335 | 0.095622483 | 0 | 0.005692914 | 0.10310819 |
| ILMN_3002505 | 0.024301667 | 0.0000941 | 0.00638343 | 0.000199793 | 0.011313575 | 1 | 0.000540727 | 0.028504193 |
| ILMN_2441724 | 0.019410925 | 0.000231088 | 0.012010162 | 0.000203189 | 0.011438788 | 1 | 0.000665544 | 0.032069437 |
| ILMN_1229005 | 0.019410925 | 0.000272708 | 0.013475313 | 0.000194981 | 0.011135911 | 1 | 0.001104043 | 0.042298211 |
| ILMN_1229518 | 0.013353714 | 0.00000774 | 0.001050295 | 0.00000785 | 0.001234703 | 1 | 9.56597E−06 | 0.002748564 |
| ILMN_2576568 | 0.048451528 | 0.002934498 | 0.06072226 | 0.005759465 | 0.08793067 | 0 | 0.004510321 | 0.091557737 |
| ILMN_2491182 | 0.152968395 | 0.004392397 | 0.075471726 | 0.018680856 | 0.168124091 | 0 | 0.037563093 | 0.2654797 |
| ILMN_2484278 | 0.051552328 | 0.005337456 | 0.085315071 | 0.009683569 | 0.116862873 | 0 | 0.012022966 | 0.149554514 |
| ILMN_2827780 | 0013353714 | 0.0000427 | 0.003546036 | 0.0000575 | 0.004674319 | 1 | 0.000642967 | 0.031573087 |
| ILMN_3112873 | 0.019410925 | 0.000143096 | 0.008611225 | 0.000243798 | 0.012729351 | 1 | 0.001141234 | 0.042874093 |
| ILMN_3117602 | 0.013353714 | 0.00000663 | 0.000949865 | 0.0000261 | 0.002837023 | 1 | 0.000197163 | 0.015477107 |
| ILMN_1233424 | 0.013353714 | 0.0000427 | 0.003546036 | 0.0000532 | 0.004485638 | 1 | 0.000103509 | 0.010612415 |
| ILMN_2681601 | 0.099378843 | 0.002543443 | 0.055844168 | 0.006876599 | 0.096328868 | 0 | 0.009432203 | 0.132484733 |
| ILMN_2762956 | 0.013353714 | 0.0000411 | 0.003468105 | 0.000131039 | 0.00859081 | 1 | 0.000189968 | 0.015089482 |
| ILMN_2632230 | 0.013353714 | 0.00000699 | 0.00098038 | 0.0000266 | 0.002856495 | 1 | 0.000317538 | 0.020788254 |
| ILMN_3052501 | 0.019410925 | 0.0000571 | 0.004360274 | 0.000117771 | 0.007898943 | 1 | 0.000966442 | 0.039356938 |
| ILMN_2615096 | 0.013353714 | 0.000000424 | 0.00011758 | 0.000000257 | 0.000106375 | 1 | 2.49646E−06 | 0.001335664 |
| ILMN_2629112 | 0.013353714 | 0.0000279 | 0.002603268 | 0.0000227 | 0.002596277 | 1 | 3.53859E−05 | 0.005861598 |
| ILMN_1254916 | 0.019410925 | 0.0000268 | 0.002529347 | 0.000291891 | 0.014258943 | 1 | 0.002106509 | 0.059970456 |
| ILMN_2686115 | 0.066594341 | 0.001242443 | 0.035534793 | 0.002860747 | 0.059099453 | 0 | 0.008219665 | 0.123658938 |
| ILMN_3153010 | 0.013353714 | 0.000158137 | 0.009109888 | 0.0000467 | 0.004101953 | 1 | 5.97852E−05 | 0.007773248 |
| ILMN_2571616 | 0.039534447 | 0.00120471 | 0.034938094 | 0.002233346 | 0.05034168 | 0 | 0.001797898 | 0.054876565 |
| ILMN_1231785 | 0.013353714 | 0.000110441 | 0.00713983 | 0.000264563 | 0.013458572 | 1 | 0.000237231 | 0.017362241 |
| ILMN_1221831 | 0.036357572 | 0.000606401 | 0.022854024 | 0.000618561 | 0.023351316 | 1 | 0.001344859 | 0.046961097 |
| ILMN_2646456 | 0.056620496 | 0.002062137 | 0.049299581 | 0.002974581 | 0.060346491 | 0 | 0.003346676 | 0.077347433 |
| ILMN_2504686 | 0.102128981 | 0.006842996 | 0.097765784 | 0.01129715 | 0.127943658 | 0 | 0.013719454 | 0.160250793 |
| ILMN_2712867 | 0.013353714 | 0.0000462 | 0.003744963 | 0.0000657 | 0.005137493 | 1 | 5.74504E−05 | 0.007618639 |
| ILMN_1234006 | 0.039534447 | 0.0000764 | 0.005463264 | 0.000342456 | 0.015876812 | 0 | 0.001104095 | 0.042298211 |
| ILMN_2965669 | 0.013353714 | 0.00000486 | 0.000741671 | 0.00000396 | 0.000746457 | 1 | 1.23984E−05 | 0.003224592 |
| ILMN_2447538 | 0.019410925 | 0.0000305 | 0.002787143 | 0.0000653 | 0.005121772 | 1 | 0.000306263 | 0.02048222 |
| ILMN_1222917 | 0.059256725 | 0.002044175 | 0.048920424 | 0.003627607 | 0.068031509 | 0 | 0.007351981 | 0.116676672 |
| ILMN_1255086 | 0.084466318 | 0.002424965 | 0.054188606 | 0.00487539 | 0.080587642 | 0 | 0.007351739 | 0.116676672 |
| ILMN_1233554 | 0.024301667 | 0.000590155 | 0.022534829 | 0.000404783 | 0.01751061 | 1 | 0.000844832 | 0.036512914 |
| ILMN_1234072 | 0.013353714 | 0.0000459 | 0.003725624 | 0.0000726 | 0.005551592 | 1 | 0.000306263 | 0.02048222 |
| ILMN_1216561 | 0.024301667 | 0.000763982 | 0.026677465 | 0.001346924 | 0.037363102 | 1 | 0.00305523 | 0.073418572 |
| ILMN_2590638 | 0.028815909 | 0.0000915 | 0.00623516 | 0.0000482 | 0.004197605 | 1 | 0.000176289 | 0.014446733 |
| ILMN_2705878 | 0.013353714 | 0.00000896 | 0.001162086 | 0.00000904 | 0.001335714 | 1 | 6.22097E−05 | 0.007890127 |
| ILMN_1227100 | 0.048451528 | 0.00208027 | 0.049529569 | 0.001352545 | 0.037429791 | 1 | 0.00040803 | 0.024132893 |
| ILMN_1254814 | 0.024301667 | 0.000229534 | 0.011977725 | 0.000414376 | 0.017763501 | 1 | 0.00099928 | 0.040236595 |
| ILMN_2986458 | 0.013353714 | 0.00036565 | 0.016444375 | 0.000252656 | 0.013009278 | 1 | 0.000470028 | 0.02607676 |
| ILMN_1239729 | 0.019410925 | 0.001377178 | 0.03773799 | 0.000992676 | 0.031178183 | 1 | 0.000504223 | 0.027322568 |
| ILMN_1217480 | 0.070737293 | 0.001177205 | 0.03450589 | 0.001708869 | 0.042787906 | 0 | 0.004379328 | 0.090517125 |
| ILMN_2652857 | 0.094076848 | 0.008284497 | 0.109488491 | 0.003876917 | 0.070436711 | 0 | 0.006383169 | 0.109238048 |
| ILMN_1257107 | 0.019410925 | 0.000540915 | 0.021355367 | 0.001177747 | 0.034456682 | 1 | 0.001179524 | 0.043782528 |
| ILMN_2636339 | 0.024301667 | 0.000297953 | 0.014283019 | 0.000767428 | 0.026757653 | 1 | 0.00270263 | 0.068855671 |
| ILMN_2479848 | 0.013353714 | 3.51E−08 | 0.0000177 | 0.000000282 | 0.000110081 | 1 | 5.89482E−06 | 0.002111595 |
| ILMN_1226514 | 0.013353714 | 0.00000136 | 0.000277495 | 0.000000237 | 0.000101599 | 1 | 6.1646E−06 | 0.002125696 |
| ILMN_3141048 | 0.024301667 | 0.000283417 | 0.013828313 | 0.000166399 | 0.009994028 | 1 | 0.000643 | 0.031573087 |
| ILMN_2635871 | 0.019410925 | 0.00015081 | 0.008859861 | 0.00020499 | 0.011473406 | 1 | 0.000643034 | 0.031573087 |
| ILMN_2829625 | 0.028815909 | 0.000147647 | 0.008754822 | 0.000417071 | 0.017826837 | 1 | 0.000438006 | 0.025077344 |
| ILMN_2635132 | 0.013353714 | 0.00000368 | 0.00061365 | 0.00000951 | 0.00136995 | 1 | 0.001301631 | 0.046179135 |
| ILMN_1225109 | 0.024301667 | 0.000231451 | 0.012010162 | 0.000463947 | 0.019281568 | 1 | 0.000559888 | 0.029151118 |
| ILMN_1228653 | 0.096526955 | 0.003640921 | 0.067632684 | 0.005932081 | 0.089284737 | 0 | 0.011706901 | 0.147795262 |
| ILMN_1236585 | 0.032842408 | 0.00010667 | 0.006973566 | 0.000361871 | 0.016337543 | 0 | 0.001797898 | 0.054876565 |
| ILMN_1227803 | 0.024301667 | 0.00000863 | 0.001137392 | 0.0000456 | 0.00403955 | 1 | 7.28705E−05 | 0.008652811 |
| ILMN_2706631 | 0.013353714 | 0.000000568 | 0.000143787 | 0.00000114 | 0.000290477 | 1 | 7.68769E−06 | 0.002401604 |
| ILMN_2949605 | 0.013353714 | 0.000000468 | 0.000126769 | 0.00000161 | 0.000370841 | 1 | 7.28705E−05 | 0.008652811 |
| ILMN_2651054 | 0.024301667 | 0.000185401 | 0.010128942 | 0.000207116 | 0.011559512 | 1 | 0.002540648 | 0.066400639 |
| ILMN_2691192 | 0.120066286 | 0.024744298 | 0.199923062 | 0.018129944 | 0.165469515 | 0 | 0.013719454 | 0.160250793 |
| ILMN_2569336 | 0.11074271 | 0.005029371 | 0.082087062 | 0.007629856 | 0.10204046 | 0 | 0.012022966 | 0.149554514 |
| ILMN_1222346 | 0.013353714 | 0.00000699 | 0.00098038 | 0.0000116 | 0.001590823 | 1 | 1.60246E−05 | 0.003674655 |
| ILMN_1255419 | 0.028815909 | 0.00023251 | 0.012025158 | 0.000461073 | 0.019196381 | 1 | 0.001633268 | 0.052249986 |
| ILMN_2704822 | 0.042916604 | 0.000737221 | 0.025996538 | 0.001507582 | 0.039781954 | 1 | 0.002041151 | 0.058756619 |
| ILMN_2595967 | 0.013353714 | 0.0000399 | 0.003374128 | 0.0000835 | 0.006132211 | 1 | 0.000353719 | 0.02219068 |
| ILMN_2628174 | 0.013353714 | 0.00000673 | 0.000954053 | 0.00000329 | 0.000651535 | 1 | 5.51982E−05 | 0.007602042 |
| ILMN_2457324 | 0.019410925 | 0.000377615 | 0.016836068 | 0.000681216 | 0.024908549 | 1 | 0.001301631 | 0.046179135 |
| ILMN_2631994 | 0.013353714 | 0.000000794 | 0.000185854 | 0.000000526 | 0.000168884 | 1 | 3.28593E−06 | 0.001544952 |
| ILMN_2544890 | 0.054324155 | 0.001446451 | 0.039053348 | 0.00467606 | 0.078680163 | 0 | 0.00388914 | 0.084081646 |
| ILMN_2696491 | 0.046026289 | 0.001306218 | 0.0366489 | 0.001777536 | 0.04368478 | 0 | 0.003888993 | 0.084081646 |
| ILMN_2775098 | 0.013353714 | 0.00000016 | 0.0000542 | 0.000000392 | 0.00013797 | 1 | 5.89742E−06 | 0.002111595 |
| ILMN_2880467 | 0.036357572 | 0.000596145 | 0.022658053 | 0.00077485 | 0.026955849 | 1 | 0.00099928 | 0.040236595 |
| ILMN_2812244 | 0.013353714 | 0.00000576 | 0.000845189 | 0.00000933 | 0.001361621 | 1 | 4.33673E−05 | 0.006596797 |
| ILMN_3133748 | 0.028815909 | 0.000026 | 0.002476668 | 0.000100197 | 0.00697141 | 1 | 0.000600122 | 0.030462238 |
| ILMN_2518828 | 0.013353714 | 0.0000988 | 0.006582067 | 0.000186034 | 0.010797146 | 1 | 0.00011618 | 0.011242898 |
| ILMN_1258965 | 0.024301667 | 0.000122032 | 0.007605138 | 0.000271158 | 0.013615504 | 1 | 0.0007895 | 0.035437666 |
| ILMN_1237886 | 0.013353714 | 0.0000265 | 0.002503706 | 0.0000379 | 0.003587283 | 1 | 0.000135361 | 0.012427306 |
| ILMN_2492102 | 0.013353714 | 0.000213393 | 0.011363803 | 0.000340257 | 0.015806312 | 1 | 0.000966442 | 0.039356938 |
| ILMN_2710166 | 0.068774915 | 0.003216093 | 0.063589034 | 0.003700581 | 0.068707988 | 0 | 0.004510321 | 0.091557737 |
| ILMN_2742928 | 0.137444039 | 0.005979984 | 0.090875061 | 0.008896378 | 0.111496961 | 0 | 0.013017012 | 0.156039878 |
| ILMN_3071525 | 0.039534447 | 0.000736131 | 0.025977764 | 0.000794336 | 0.027327392 | 0 | 0.001977658 | 0.05767797 |
| ILMN_2720820 | 0.013353714 | 0.00000291 | 0.D00513168 | 0.0000077 | 0.001222667 | 1 | 8.19763E−05 | 0.009306714 |
| ILMN_1238069 | 0.013353714 | 0.0000032 | 0.000552276 | 0.0000217 | 0.002508955 | 1 | 0.000157497 | 0.013500967 |
| ILMN_1238285 | 0.032842408 | 0.000348114 | 0.015948481 | 0.000289089 | 0.014164441 | 1 | 0.000284812 | 0.019439747 |
| ILMN_1234133 | 0.019410925 | 0.000319996 | 0.015015535 | 0.000495252 | 0.020203747 | 1 | 0.000284828 | 0.019439747 |
| ILMN_1217705 | 0.11074271 | 0.008238647 | 0.109099373 | 0.010773072 | 0.124430321 | 0 | 0.015225452 | 0.168938998 |
| ILMN_2614966 | 0.013353714 | 8.05E−08 | 0.0000329 | 2.31E−08 | 0.0000187 | 1 | 1.2393E−06 | 0.000901339 |
| ILMN_1245393 | 0.042916604 | 0.001152702 | 0.034218493 | 0.001900889 | 0.045691414 | 0 | 0.00553098 | 0.101481885 |
| ILMN_1239726 | 0.019410925 | 0.0000322 | 0.002892763 | 0.000113724 | 0.007705253 | 1 | 0.000665544 | 0.032069437 |
| ILMN_1223028 | 0.019410925 | 0.000286457 | 0.013889283 | 0.000348862 | 0.016068328 | 1 | 0.000873828 | 0.037145252 |
| ILMN_2854878 | 0.013353714 | 0.00000877 | 0.001147985 | 0.0000229 | 0.002603706 | 1 | 8.19763E−05 | 0.009306714 |
| ILMN_1242802 | 0.019410925 | 0.000613933 | 0.022953221 | 0.000318873 | 0.015112162 | 1 | 0.001856078 | 0.055774594 |
| ILMN_2535779 | 0.024301667 | 0.00056898 | 0.02197867 | 0.00070261 | 0.025381142 | 1 | 0.001104095 | 0.042298211 |
| ILMN_1228235 | 0.024301667 | 0.000290789 | 0.014070003 | 0.000184022 | 0.010707081 | 1 | 0.00057968 | 0.029683566 |
| ILMN_3163163 | 0.019410925 | 0.00013728 | 0.008352935 | 0.00013654 | 0.008839388 | 1 | 0.00022037 | 0.016464793 |
| ILMN_2732190 | 0.061967633 | 0.001140652 | 0.034012759 | 0.00113476 | 0.033772175 | 0 | 0.013719454 | 0.160250793 |
| ILMN_1246770 | 0.051552328 | 0.003584735 | 0.067314254 | 0.003165165 | 0.062746493 | 0 | 0.005858095 | 0.104408326 |
| ILMN_1250135 | 0.019410925 | 0.0000434 | 0.00359485 | 0.0000827 | 0.006091257 | 1 | 0.000329201 | 0.021282383 |
| ILMN_3114641 | 0.013353714 | 0.00000126 | 0.000263891 | 0.00000119 | 0.00029799 | 1 | 1.47177E−05 | 0.003531258 |
| ILMN_2677056 | 0.039534447 | 0.002464159 | 0.054794935 | 0.002601189 | 0.055447887 | 0 | 0.003774648 | 0.082954936 |
| ILMN_1243042 | 0.013353714 | 0.00000214 | 0.000401503 | 0.0000184 | 0.002263583 | 1 | 4.33673E−05 | 0.006596797 |
| ILMN_2917338 | 0.054324155 | 0.002100432 | 0.049831197 | 0.003394382 | 0.065464235 | 0 | 0.00270263 | 0.068855671 |
| ILMN_2433964 | 0.032842408 | 0.000471174 | 0.01946027 | 0.000359654 | 0.0162751 | 1 | 0.00057968 | 0.029683566 |
| ILMN_2566477 | 0.113383658 | 0.009538032 | 0.118422719 | 0.011738911 | 0.130774947 | 0 | 0.012346572 | 0.151955545 |
| ILMN_2704823 | 0.028815909 | 0.000991032 | 0.031274283 | 0.000594473 | 0.022718354 | 1 | 0.001104095 | 0.042298211 |
| ILMN_1249863 | 0.024301667 | 0.000531621 | 0.021116652 | 0.000185389 | 0.010773166 | 1 | 0.001686537 | 0.053258626 |
| ILMN_2617820 | 0.013353714 | 0.000000555 | 0.000142642 | 0.000000719 | 0.000216047 | 1 | 1.47177E−05 | 0.003531258 |
| ILMN_1238109 | 0.013353714 | 0.000103622 | 0.006822227 | 0.0000106 | 0.001483462 | 1 | 3.68613E−05 | 0.005999236 |
| ILMN_1257463 | 0.024301667 | 0.000647324 | 0.023781354 | 0.000296444 | 0.014423405 | 1 | 0.001531347 | 0.050161589 |
| ILMN_2634157 | 0013353714 | 0.00000507 | 0.000764406 | 0.0000188 | 0.002298746 | 1 | 3.12891E−05 | 0.005434371 |
| ILMN_1238869 | 0.048451528 | 0.000637697 | 0.023592972 | 0.001216774 | 0.035196389 | 0 | 0.00305523 | 0.073418572 |
| ILMN_1215910 | 0.013353714 | 0.000745879 | 0.026123729 | 0.00053798 | 0.021230722 | 1 | 0.000438006 | 0.025077344 |
| ILMN_2621086 | 0.032842408 | 0.001471072 | 0.039466269 | 0.000703549 | 0.025381142 | 1 | 0.000873828 | 0.037145252 |
| ILMN_2699531 | 0.032842408 | 0.000519887 | 0.020807554 | 0.000412208 | 0.017749337 | 1 | 0.001068077 | 0.041672924 |
| ILMN_2603187 | 0.013353714 | 0.00000123 | 0.000260429 | 0.00000145 | 0.000347545 | 1 | 5.39498E−06 | 0.002025163 |
| ILMN_2982781 | 0.019410925 | 0.0000223 | 0.00222523 | 0.0000282 | 0.002959627 | 1 | 0.000151664 | 0.013244851 |
| ILMN_1244845 | 0.024301667 | 0.0000508 | 0.003974909 | 0.000159623 | 0.00973796 | 1 | 0.000453754 | 0.02560106 |
| ILMN_2680398 | 0.013353714 | 0.000001 | 0.000219086 | 0.000000952 | 0.000257701 | 1 | 7.03927E−06 | 0.002307643 |
| ILMN_2605031 | 0.098004431 | 0.004537122 | 0.077076423 | 0.012645685 | 0.136133148 | 0 | 0.010802479 | 0.142201077 |
| ILMN_2617477 | 0.028815909 | 0.000157886 | 0.009106733 | 0.000173663 | 0.010310561 | 1 | 0.00010758 | 0.010838827 |
| ILMN_2901284 | 0.013353714 | 0.0000471 | 0.003800848 | 0.0000359 | 0.003445285 | 1 | 4.70154E−05 | 0.006881837 |
| ILMN_2506499 | 0.046026289 | 0.000531693 | 0.021116652 | 0.001925925 | 0.046019516 | 1 | 0.004924874 | 0.095356944 |
| ILMN_2605960 | 0.032842408 | 0.000428022 | 0.018375605 | 0.000589526 | 0.022608846 | 1 | 0001141234 | 0.042874093 |
| ILMN_2562725 | 0.024301667 | 0.000877065 | 0.028689223 | 0.001021509 | 0.031783537 | 1 | 0.000504223 | 0.027322568 |
| ILMN_2567626 | 0.076777595 | 0.006420983 | 0.094342641 | 0.004575698 | 0.077674245 | 0 | 0.012677869 | 0.153956896 |
| ILMN_2954868 | 0.051552328 | 0.004951444 | 0.081231057 | 0.002127551 | 0.048753581 | 0 | 0.0010331 | 0.040997434 |
| ILMN_1233894 | 0.013353714 | 0.000394741 | 0.017353743 | 0.001002721 | 0.0313667 | 1 | 0.00057968 | 0.029683566 |
| ILMN_1229245 | 0.036357572 | 0.000446402 | 0.018841131 | 0.000624846 | 0.023550381 | 1 | 0.000873828 | 0.037145252 |
| ILMN_1256639 | 0.013353714 | 0.0000315 | 0.002856803 | 0.0000379 | 0.003587283 | 1 | 0.000353739 | 0.02219068 |
| ILMN_2631192 | 0.042916604 | 0.002437285 | 0.054372505 | 0.001151219 | 0.03404972 | 0 | 0.006029251 | 0.106465686 |
| ILMN_2038768 | 0.397349446 | 0.117598066 | 0.433296696 | 0.11009287 | 0.413665062 | 0 | 0.246250128 | 0.607563618 |
| ILMN_2668977 | 0.013353714 | 0.00000386 | 0.000632356 | 0.00000566 | 0.00097714 | 1 | 1.1877E−05 | 0.003141395 |
| ILMN_1248211 | 0.039534447 | 0.001158917 | 0.034271991 | 0.001607153 | 0.041193921 | 0 | 0.002873882 | 0.071309138 |
| ILMN_2534151 | 0.0826602 | 0.030053192 | 0.221517959 | 0.009055427 | 0.112610992 | 0 | 0.007994557 | 0.121608701 |
| ILMN_1224211 | 0.088252989 | 0.007964394 | 0.107082218 | 0.015471566 | 0.152284836 | 0 | 0.023367993 | 0.210226124 |
| ILMN_2771182 | 0.036357572 | 0.000482252 | 0.019777416 | 0.000254672 | 0.01309856 | 1 | 0.003246828 | 0.076021173 |
| ILMN_2890357 | 0.019410925 | 0.000448658 | 0.018899281 | 0.000401323 | 0.017418469 | 1 | 0.000559888 | 0.029151118 |
| ILMN_2705732 | 0.028815909 | 0.000260587 | 0.013056566 | 0.000223062 | 0.012129499 | 1 | 0.000559888 | 0.029151118 |
| ILMN_2422023 | 0.07282894 | 0.005293782 | 0.084851812 | 0.006492934 | 0.093577166 | 0 | 0.007351981 | 0.116676672 |
| ILMN_1239398 | 0.179616163 | 0.010052171 | 0.122378526 | 0.025572066 | 0.198492675 | 0 | 0.044021446 | 0.284999621 |
| ILMN_1256245 | 0.095217785 | 0.003635324 | 0.067604241 | 0.008632236 | 0.109699863 | 0 | 0.005373592 | 0.100089876 |
| ILMN_1243846 | 0.032842408 | 0.00073562 | 0.025977764 | 0.002381707 | 0.052317743 | 0 | 0.001633268 | 0.052249986 |
| ILMN_1242769 | 0.056620496 | 0.001845509 | 0.045888316 | 0.002962195 | 0.060255548 | 0 | 0.002387591 | 0.063870812 |
| ILMN_2993109 | 0.013353714 | 0.00000961 | 0.001211954 | 0.000042 | 0.003865058 | 1 | 0.000146035 | 0.012922953 |
| ILMN_1245987 | 0.197371412 | 0.02738494 | 0.210925532 | 0.04211374 | 0.257862175 | 0 | 0.051397104 | 0.307424957 |

Probes with |Fold Change| <1.10 not displayed

| | | | | | | | | |
|---|---|---|---|---|---|---|---|---|
| ILMN_2715198 | 0.094076848 | 0.010940387 | 0.128401963 | 0.009461691 | 0.115200974 | 0 | 0.008929411 | 0.129240455 |
| ILMN_2711045 | 0.024301667 | 0.000484665 | 0.019858893 | 0.000269243 | 0.013583836 | 1 | 0.000264806 | 0.018619193 |
| ILMN_2734856 | 0.013353714 | 0.00000347 | 0.000586923 | 0.00000793 | 0.001243596 | 1 | 1.97985E−05 | 0.004208042 |
| ILMN_1253854 | 0.102128981 | 0.004275639 | 0.074288225 | 0.006437968 | 0.093170157 | 0 | 0.017311316 | 0.180185556 |
| ILMN_2769945 | 0.024301667 | 0.0000879 | 0.006058155 | 0.000203233 | 0.011438788 | 1 | 0.000522178 | 0.028002086 |
| ILMN_2954195 | 0.084466318 | 0.003549564 | 0.066945529 | 0.006164384 | 0.090932677 | 0 | 0.009177877 | 0.130483403 |
| ILMN_2988572 | 0.080759553 | 0.001631327 | 0.0423263 | 0.00517364 | 0.083183907 | 0 | 0.011706901 | 0.147795262 |
| ILMN_2728118 | 0.013353714 | 0.0000058 | 0.000845189 | 0.00000976 | 0.001398167 | 1 | 2.34093E−05 | 0.00450296 |
| ILMN_1235470 | 0.013353714 | 0.000000028 | 0.0000148 | 0.000000142 | 0.0000709 | 1 | 4.50906E−06 | 0.001809827 |
| ILMN_2774410 | 0.236732645 | 0.035070686 | 0.240476898 | 0.039755719 | 0.250087018 | 0 | 0.058515334 | 0.326349541 |
| ILMN_2856095 | 0.024301667 | 0.00016088 | 0.009199591 | 0.000330427 | 0.015473218 | 1 | 0.001259689 | 0.045559243 |
| ILMN_2985447 | 0.024301667 | 0.000360066 | 0.016271848 | 0.000308751 | 0.014785413 | 1 | 0.000438006 | 0.025077344 |
| ILMN_2870487 | 0.036357572 | 0.000156883 | 0.009060097 | 0.000338388 | 0.015735209 | 0 | 0.002173782 | 0.060770595 |
| ILMN_2736762 | 0.102128981 | 0.004273519 | 0.074279126 | 0.006779044 | 0.095608851 | 0 | 0.021171123 | 0.199686371 |
| ILMN_2620061 | 0.013353714 | 0.0000296 | 0.002740308 | 0.0000316 | 0.003180587 | 1 | 9.5799E−05 | 0.010180718 |
| ILMN_1252016 | 0.019410925 | 0.0000452 | 0.003705003 | 0.00013411 | 0.00874694 | 1 | 0.000453754 | 0.02560106 |
| ILMN_2683802 | 0.013353714 | 0.00000633 | 0.000915234 | 0.0000127 | 0.001692242 | 1 | 7.57938E−05 | 0.008797941 |
| ILMN_2758087 | 0.054324155 | 0.001171168 | 0.034442996 | 0.001373589 | 0.03783222 | 0 | 0.003663228 | 0.081662975 |
| ILMN_1222246 | 0.099378843 | 0.002890957 | 0.060142799 | 0.005204118 | 0.083438223 | 0 | 0.013017012 | 0.156039878 |
| ILMN_1250149 | 0.013353714 | 0.0000385 | 0.003287434 | 0.000081 | 0.006004936 | 1 | 0.000135361 | 0.012427306 |
| ILMN_2855423 | 0.066594341 | 0.000975784 | 0.0310015 | 0.00261887 | 0.055581773 | 0 | 0.011096803 | 0.144280139 |
| ILMN_3093150 | 0.121464806 | 0.007488533 | 0.103432865 | 0.010780409 | 0.12447925 | 0 | 0.018679697 | 0.18694557 |
| ILMN_2887208 | 0.068774915 | 0.002114263 | 0.049983662 | 0.001621353 | 0.041443788 | 0 | 0.005220076 | 0.098611557 |
| ILMN_1246622 | 0.019410925 | 0.0000832 | 0.005824704 | 0.0000636 | 0.005055758 | 1 | 0.000246108 | 0.017705716 |
| ILMN_1237507 | 0.013353714 | 0.0000239 | 0.0023395 | 0.0000261 | 0.002837023 | 1 | 4.70154E−05 | 0.006881837 |
| ILMN_2615739 | 0.168667397 | 0.019024292 | 0.174935542 | 0.028152145 | 0.207735874 | 0 | 0.018679697 | 0.18694557 |
| ILMN_2605767 | 0.013353714 | 0.000478648 | 0.019631646 | 0.0000565 | 0.004637506 | 1 | 0.000120719 | 0.011491043 |
| ILMN_2675033 | 0.013353714 | 0.0000189 | 0.001948172 | 0.0000376 | 0.003576126 | 1 | 9.5799E−05 | 0.010180718 |
| ILMN_2531520 | 0.019410925 | 0.0000441 | 0.003636842 | 0.0000899 | 0.006457872 | 1 | 0.000393771 | 0.023711359 |
| ILMN_2952587 | 0.125338424 | 0.006468173 | 0.094796612 | 0.00750925 | 0.101343301 | 0 | 0.008450429 | 0.125387993 |
| ILMN_3162081 | 0.039534447 | 0.000474019 | 0.019543082 | 0.000886286 | 0.029245777 | 1 | 0.000934606 | 0.038635069 |
| ILMN_2823778 | 0.013353714 | 0.000609634 | 0.022902836 | 0.000353682 | 0.016129651 | 1 | 0.000176289 | 0.014446733 |
| ILMN_2637804 | 0.199347911 | 0.033358108 | 0.234514405 | 0.027081488 | 0.204795156 | 0 | 0.03192796 | 0.244754736 |
| ILMN_2944939 | 0.013353714 | 0.0000298 | 0.002749089 | 0.0000185 | 0.002263791 | 1 | 3.12891E−05 | 0.005434371 |
| ILMN_2966386 | 0.019410925 | 0.00000151 | 0.000303563 | 0.00000531 | 0.000929895 | 1 | 8.86216E−05 | 0.009822905 |
| ILMN_2721208 | 0.07282894 | 0.000875964 | 0.028689223 | 0.001541191 | 0.04014427 | 0 | 0.005531174 | 0.101481885 |
| ILMN_1243107 | 0.019410925 | 0.0000139 | 0.001584056 | 0.0000239 | 0.002688276 | 1 | 0.000204639 | 0.01592867 |
| ILMN_2491232 | 0.032842408 | 0.000160026 | 0.009162023 | 0.000178437 | 0.010486465 | 1 | 0.003055109 | 0.073418572 |
| ILMN_1259100 | 0.051552328 | 0.000884632 | 0.028896103 | 0.001952301 | 0.046411523 | 0 | 0.002173782 | 0.060770595 |
| ILMN_1225747 | 0.013353714 | 0.0000072 | 0.000992417 | 0.0000278 | 0.002931426 | 1 | 0.000453754 | 0.02560106 |
| ILMN_2721149 | 0.056620496 | 0.000781551 | 0.026967268 | 0.000916667 | 0.029754618 | 0 | 0.002620495 | 0.067689332 |
| ILMN_1244424 | 0.046026289 | 0.000704457 | 0.025331453 | 0.000660708 | 0.024405921 | 0 | 0.00388914 | 0.084081646 |
| ILMN_1228832 | 0.113383658 | 0.004145932 | 0.073302473 | 0.004001218 | 0.071825953 | 0 | 0.011398229 | 0.146077469 |
| ILMN_1260456 | 0.013353714 | 0.000000864 | 0.000199042 | 0.00000221 | 0.000472255 | 1 | 2.15311E−05 | 0.004320203 |
| ILMN_1227235 | 0.028815909 | 0.000353703 | 0.016125168 | 0.000567099 | 0.0219992 | 1 | 0.000763113 | 0.034824155 |
| ILMN_2871749 | 0.070737293 | 0.009625128 | 0.119186174 | 0.006414918 | 0.093049294 | 0 | 0.005220076 | 0.098611557 |
| ILMN_2911344 | 0.013353714 | 0.000551745 | 0.021563464 | 0.000115271 | 0.007776142 | 1 | 0.000329201 | 0.021282383 |
| ILMN_2698494 | 0.028815909 | 0.001011866 | 0.031674068 | 0.001309443 | 0.036673066 | 1 | 0.001301631 | 0.046179135 |
| ILMN_2603898 | 0.019410925 | 0.000477241 | 0.01964113 | 0.000139187 | 0.008973291 | 1 | 0.00042277 | 0.024660011 |
| ILMN_2749448 | 0.032842408 | 0.0000583 | 0.004418055 | 0.000178021 | 0.010475755 | 1 | 0.000600091 | 0.030462238 |
| ILMN_2631014 | 0.013353714 | 0.00000858 | 0.001133948 | 0.0000309 | 0.003134864 | 1 | 3.39668E−05 | 0.00570777 |
| ILMN_2595863 | 0.013353714 | 0.000116386 | 0.007431321 | 0.000303564 | 0.01463008 | 1 | 0.00022037 | 0.016464793 |
| ILMN_1231439 | 0.032842408 | 0.0000541 | 0.004165288 | 0.000259313 | 0.013278569 | 1 | 0.003449313 | 0.078819425 |
| ILMN_2923607 | 0.013353714 | 0.0000359 | 0.003145034 | 0.0000324 | 0.003223303 | 1 | 5.09531E−05 | 0.007252947 |
| ILMN_2544343 | 0.013353714 | 0.00000574 | 0.000845189 | 0.0000233 | 0.002627969 | 1 | 0.000176289 | 0.014446733 |
| ILMN_1238102 | 0.289534001 | 0.037803431 | 0.249381564 | 0.066367866 | 0.321827801 | 0 | 0.059776798 | 0.328820555 |
| ILMN_2834198 | 0.019410925 | 0.000237665 | 0.012175724 | 0.000446487 | 0.018790822 | 1 | 0.000453754 | 0.02560106 |
| ILMN_1236702 | 0.102128981 | 0.002240682 | 0.051760317 | 0.003875331 | 0.070435395 | 0 | 0.017311316 | 0.180185556 |
| ILMN_2979639 | 0.039534447 | 0.001726536 | 0.043915331 | 0.000996725 | 0.031258932 | 1 | 0.00270263 | 0.068855671 |
| ILMN_1256817 | 0.20371558 | 0.013221865 | 0.143251676 | 0.016541231 | 0.157798872 | 0 | 0.038432634 | 0.26812964 |
| ILMN_2590350 | 0.024301667 | 0.00030653 | 0.014534302 | 0.000065 | 0.005110515 | 1 | 0.000157497 | 0.013500967 |
| ILMN_1240318 | 0.013353714 | 0.00037337 | 0.016697112 | 0.000524897 | 0.020878705 | 1 | 0.000470028 | 0.02607676 |
| ILMN_1215893 | 0.013353714 | 0.00000791 | 0.001060486 | 0.00000859 | 0.001299233 | 1 | 0.000212368 | 0.016047225 |
| ILMN_2780915 | 0.028815909 | 0.000436629 | 0.018614041 | 0.000675195 | 0.024769672 | 1 | 0.002387492 | 0.063870812 |
| ILMN_2746830 | 0.039534447 | 0.00012205 | 0.007605138 | 0.000874003 | 0.02899696 | 0 | 0.005858907 | 0.104408326 |
| ILMN_3120014 | 0.112323558 | 0.012670518 | 0.139823277 | 0.013221949 | 0.139651023 | 0 | 0.029744406 | 0.23776625 |
| ILMN_2959292 | 0.098004431 | 0.006166693 | 0.092231529 | 0.007371792 | 0.100219589 | 0 | 0.008450429 | 0.125387993 |
| ILMN_2633457 | 0.019410925 | 0.0002621 | 0.013118251 | 0.000180465 | 0.010552902 | 1 | 0.000688785 | 0.032681842 |
| ILMN_2646209 | 0.106524046 | 0.00837294 | 0.109931638 | 0.005970714 | 0.089478328 | 0 | 0.004644853 | 0.093111105 |
| ILMN_2680440 | 0.024301667 | 0.0000173 | 0.001831837 | 0.000154786 | 0.009492492 | 1 | 0.00057965 | 0.029683566 |
| ILMN_2691706 | 0.039534447 | 0.000594509 | 0.022658053 | 0.00187868 | 0.045262385 | 1 | 0.001531347 | 0.050161589 |
| ILMN_1241909 | 0.013353714 | 0.000124699 | 0.007749516 | 0.000235699 | 0.012495554 | 1 | 0.001301631 | 0.046179135 |
| ILMN_2686975 | 0.019410925 | 0.000100156 | 0.006650425 | 0.0000642 | 0.005083088 | 1 | 0.000621233 | 0.03109305 |
| ILMN_2900557 | 0.046026289 | 0.000508153 | 0.020532133 | 0.000737458 | 0.02616347 | 0 | 0.002314175 | 0.062958735 |
| ILMN_2745954 | 0.013353714 | 0.000257427 | 0.012954007 | 0.000267465 | 0.013561762 | 1 | 0.000284828 | 0.019439747 |
| ILMN_1226372 | 0.019410925 | 0.0000159 | 0.001744691 | 0.000055 | 0.004568149 | 1 | 0.000379979 | 0.02324165 |
| ILMN_3084818 | 0.028815909 | 0.000421067 | 0.018198145 | 0.000978894 | 0.030933174 | 1 | 0.001797898 | 0.054876565 |
| ILMN_1248733 | 0.046026289 | 0.002259152 | 0.051954924 | 0.003953198 | 0.071295686 | 0 | 0.002787111 | 0.070087328 |
| ILMN_2630605 | 0.080759553 | 0.0018293 | 0.045607075 | 0.002556189 | 0.054799056 | 0 | 0.007351981 | 0.116676672 |
| ILMN_3003631 | 0.048451528 | 0.000994427 | 0.031329141 | 0.00125441 | 0.035750241 | 0 | 0.000934606 | 0.038635069 |
| ILMN_2598775 | 0.032842408 | 0.0000601 | 0.004524475 | 0.000153316 | 0.009469751 | 1 | 0.000737547 | 0.034262098 |
| ILMN_2588995 | 0.039534447 | 0.000610966 | 0.022924563 | 0.000681824 | 0.024911182 | 0 | 0.00099904 | 0.040236595 |
| ILMN_2936468 | 0.013353714 | 0.000000848 | 0.000196389 | 0.00000286 | 0.000576709 | 1 | 3.26018E−05 | 0.005579105 |
| ILMN_2699522 | 0.013353714 | 0.0000346 | 0.003062528 | 0.0000326 | 0.003233591 | 1 | 2.65234E−05 | 0.004803829 |
| ILMN_2611431 | 0.177055003 | 0.030858102 | 0.224426141 | 0.030826222 | 0.218815938 | 0 | 0.043046488 | 0.281931179 |
| ILMN_2908056 | 0.013353714 | 0.000355587 | 0.016178372 | 0.000702712 | 0.025381142 | 1 | 0.000844832 | 0.036512914 |
| ILMN_2744909 | 0.019410925 | 0.002181893 | 0.050984232 | 0.001601194 | 0.041131766 | 0 | 0.001218998 | 0.044502503 |
| ILMN_2925281 | 0.013353714 | 0.000000143 | 0.0000497 | 0.000000183 | 0.0000833 | 1 | 1.89069E−06 | 0.001114107 |
| ILMN_2988931 | 0.144430879 | 0.0128175 | 0.140645018 | 0.014257192 | 0.145852642 | 0 | 0.022800994 | 0.207572432 |
| ILMN_2819679 | 0.156283296 | 0.018816881 | 0.174028972 | 0.018978511 | 0.169539906 | 0 | 0.032684998 | 0.247385688 |
| ILMN_2698430 | 0.024301667 | 0.000842975 | 0.02806722 | 0.00013557 | 0.008801061 | 1 | 0.000621233 | 0.03109305 |
| ILMN_3137980 | 0.013353714 | 0.0000051 | 0.000765509 | 0.00000777 | 0.001225434 | 1 | 3.25994E−05 | 0.005579105 |
| ILMN_2914507 | 0.0826602 | 0.003234751 | 0.063747608 | 0.004757926 | 0.079602307 | 0 | 0.007994557 | 0.121608701 |
| ILMN_1243329 | 0.096526955 | 0.0024452 | 0.054509936 | 0.003116238 | 0.062120568 | 0 | 0.029045675 | 0.234883436 |
| ILMN_2640453 | 0.019410925 | 0.0000894 | 0.006126826 | 0.000120544 | 0.008038629 | 1 | 0.000237231 | 0.017362241 |
| ILMN_2867696 | 0.013353714 | 0.0000456 | 0.003724783 | 0.000110702 | 0.007522402 | 1 | 0.000379979 | 0.02324165 |
| ILMN_2795412 | 0.019410925 | 0.0000316 | 0.002858552 | 0.0000256 | 0.002802168 | 1 | 8.86334E−05 | 0.009822905 |
| ILMN_2727273 | 0.013353714 | 0.00000132 | 0.000271671 | 0.0000026 | 0.000530708 | 1 | 2.44059E−05 | 0.00463717 |
| ILMN_2620767 | 0.013353714 | 0.00000256 | 0.000464931 | 0.00000565 | 0.00097714 | 1 | 4.33673E−05 | 0.006596797 |
| ILMN_1221920 | 0.013353714 | 0.0000203 | 0.002081518 | 0.0000086 | 0.001299233 | 1 | 1.74444E−05 | 0.003903768 |
| ILMN_2764607 | 0.013353714 | 0.00000473 | 0.000733297 | 0.0000076 | 0.001211113 | 1 | 4.89467E−05 | 0.007053629 |
| ILMN_2842767 | 0.019410925 | 0.000238036 | 0.012175724 | 0.000226352 | 0.012236289 | 1 | 0.00042277 | 0.024660011 |
| ILMN_2755399 | 0.042916604 | 0.002129304 | 0.050174412 | 0.00148083 | 0.039320129 | 0 | 0.001531347 | 0.050161589 |
| ILMN_1256354 | 0.024301667 | 0.0000865 | 0.005995631 | 0.0000573 | 0.004664893 | 1 | 7.57938E−05 | 0.008797941 |
| ILMN_2981689 | 0.013353714 | 0.000013 | 0.00149316 | 0.00000117 | 0.000296995 | 1 | 7.35697E−06 | 0.002329557 |
| ILMN_2915303 | 0.028815909 | 0.000130601 | 0.007988263 | 0.000242484 | 0.012705581 | 1 | 0.000228655 | 0.016840488 |
| ILMN_1215209 | 0.164948412 | 0.01231585 | 0.137285353 | 0.014561252 | 0.147630924 | 0 | 0.016448649 | 0.176089072 |
| ILMN_2841290 | 0.019410925 | 0.000308378 | 0.014587484 | 0.000187938 | 0.010853565 | 1 | 0.000212368 | 0.016047225 |
| ILMN_2959293 | 0.032842408 | 0.001009941 | 0.031635071 | 0.001351043 | 0.037410476 | 1 | 0.002314272 | 0.062958735 |
| ILMN_2614563 | 0.013353714 | 0.0000444 | 0.003643784 | 0.000141719 | 0.009037925 | 1 | 0.000237231 | 0.017362241 |
| ILMN_3160203 | 0.019410925 | 0.0000159 | 0.001744691 | 0.0000628 | 0.00500903 | 1 | 0.000306263 | 0.02048222 |
| ILMN_2601379 | 0.013353714 | 0.0000105 | 0.001281506 | 0.0000199 | 0.002370654 | 1 | 4.51564E−05 | 0.006758509 |
| ILMN_1235000 | 0.019410925 | 0.0000128 | 0.001486029 | 0.00000478 | 0.000855756 | 1 | 3.83949E−05 | 0.006099544 |
| ILMN_2640995 | 0.013353714 | 0.000000143 | 0.0000497 | 0.000000559 | 0.000175929 | 1 | 5.39498E−06 | 0.002025163 |
| ILMN_2884098 | 0.013353714 | 0.00000784 | 0.001059356 | 0.0000312 | 0.003152395 | 1 | 7.28705E−05 | 0.008652811 |
| ILMN_1256142 | 0.036357572 | 0.0000741 | 0.005348334 | 0.000261016 | 0.01332625 | 0 | 0.000643034 | 0.031573087 |
| ILMN_2601147 | 0.039534447 | 0.000534729 | 0.021219104 | 0.000786349 | 0.027233771 | 1 | 0.001581552 | 0.051264971 |
| ILMN_2680549 | 0.013353714 | 0.000250228 | 0.012673975 | 0.000458429 | 0.01916188 | 1 | 0.001686462 | 0.053258626 |
| ILMN_1218181 | 0.028815909 | 0.000942915 | 0.030164849 | 0.000377375 | 0.0167608 | 1 | 0.000643034 | 0.031573087 |
| ILMN_2550095 | 0.036357572 | 0.000813454 | 0.027557393 | 0.001954544 | 0.046417419 | 1 | 0.003149701 | 0.074838758 |
| ILMN_1224390 | 0.013353714 | 0.00000447 | 0.000708724 | 0.0000272 | 0.002888688 | 1 | 0.000189968 | 0.015039482 |
| ILMN_1240746 | 0.036357572 | 0.000112299 | 0.007249896 | 0.000286432 | 0.014095117 | 0 | 0.002173782 | 0.060770595 |
| ILMN_1230423 | 0.013353714 | 0.0000155 | 0.001716578 | 0.00000487 | 0.000862773 | 1 | 7.88278E−05 | 0.009015226 |
| ILMN_1247468 | 0.013353714 | 0.0000521 | 0.004045058 | 0.0000644 | 0.005086516 | 1 | 6.47271E−05 | 0.008034278 |
| ILMN_3140913 | 0.013353714 | 0.0000389 | 0.00330101 | 0.00000834 | 0.001279253 | 1 | 0.000111802 | 0.010979022 |
| ILMN_2519673 | 0.013353714 | 0.0000321 | 0.002890742 | 0.0000414 | 0.003820132 | 1 | 9.21504E−05 | 0.009952034 |
| ILMN_2617468 | 0.024301667 | 0.001006775 | 0.031562764 | 0.000392342 | 0.017180018 | 1 | 0.000665544 | 0.032069437 |
| ILMN_2450031 | 0.024301667 | 0.0000097 | 0.001217617 | 0.0000309 | 0.003134864 | 1 | 3.68613E−05 | 0.005999236 |
| ILMN_2450155 | 0.013353714 | 0.00000122 | 0.000258815 | 0.0000337 | 0.003292439 | 1 | 0.000317448 | 0.020788254 |
| ILMN_1218104 | 0.013353714 | 0.00000346 | 0.000586923 | 0.00000396 | 0.000746457 | 1 | 5.74504E−05 | 0.007618639 |
| ILMN_1237743 | 0.013353714 | 0.000000654 | 0.000160103 | 0.00000198 | 0.000436891 | 1 | 1.08955E−05 | 0.00300091 |
| ILMN_1222872 | 0.061967633 | 0.001378275 | 0.03773799 | 0.003620373 | 0.068005458 | 0 | 0.004924874 | 0.095356944 |
| ILMN_2687828 | 0.095217785 | 0.002788554 | 0.059026289 | 0.003542367 | 0.067235737 | 0 | 0.011096803 | 0.144280139 |
| ILMN_2772492 | 0.013353714 | 0.0000835 | 0.00583655 | 0.0000958 | 0.0067548 | 1 | 0.000146035 | 0.012922953 |
| ILMN_2868480 | 0.328827487 | 0.059774116 | 0.312050787 | 0.079523141 | 0.35376248 | 0 | 0.035872649 | 0.258477426 |
| ILMN_1219168 | 0.013353714 | 0.000122008 | 0.007605138 | 0.000459126 | 0.01916188 | 1 | 0.000621233 | 0.03109305 |
| ILMN_1214608 | 0.019410925 | 0.0000167 | 0.001787628 | 0.0000626 | 0.00500138 | 1 | 0.000816732 | 0.036034504 |
| ILMN_2723708 | 0.013353714 | 0.00000754 | 0.001031824 | 0.0000151 | 0.001938491 | 1 | 5.09531E−05 | 0.007252947 |
| ILMN_2806180 | 0.032842408 | 0.000121883 | 0.007605138 | 0.000345469 | 0.015942739 | 1 | 0.001977658 | 0.05767797 |
| ILMN_2652172 | 0.024301667 | 0.0000386 | 0.003287434 | 0.000106245 | 0.007284834 | 1 | 0.000212368 | 0.016047225 |
| ILMN_2859847 | 0.032842408 | 0.000101181 | 0.00668036 | 0.000377902 | 0.0167608 | 1 | 0.001482614 | 0.049612679 |
| ILMN_2470360 | 0.013353714 | 0.00000126 | 0.000263891 | 0.00000148 | 0.000354054 | 1 | 9.15833E−06 | 0.002681086 |
| ILMN_1217040 | 0.056620496 | 0.001718083 | 0.04381888 | 0.003497571 | 0.066721909 | 0 | 0.005373592 | 0.100089876 |
| ILMN_1223285 | 0.013353714 | 0.000000797 | 0.000185854 | 0.00000345 | 0.000677604 | 1 | 4.70154E−05 | 0.006881837 |
| ILMN_2950814 | 0.074974418 | 0.000814439 | 0.027570687 | 0.002319092 | 0.051476756 | 0 | 0.004783009 | 0.09409752 |
| ILMN_1214952 | 0.013353714 | 0.00000672 | 0.000954053 | 0.0000454 | 0.004033989 | 1 | 6.73408E−05 | 0.00831435 |
| ILMN_3163504 | 0.013353714 | 0.0000174 | 0.001832586 | 0.00000733 | 0.001185124 | 1 | 5.09531E−05 | 0.007252947 |
| ILMN_2822552 | 0.028815909 | 0.000774556 | 0.026825337 | 0.002334851 | 0.051588335 | 0 | 0.00270263 | 0.068855671 |
| ILMN_2717678 | 0.013353714 | 0.00000878 | 0.001147985 | 0.0000157 | 0.00199766 | 1 | 5.97852E−05 | 0.007773248 |
| ILMN_2433547 | 0.013353714 | 0.00000111 | 0.000240925 | 0.00000369 | 0.000720943 | 1 | 3.53859E−05 | 0.005861598 |
| ILMN_2788073 | 0.061967633 | 0.001023516 | 0.0318673 | 0.001502144 | 0.039708565 | 0 | 0.001977658 | 0.05767797 |
| ILMN_1219208 | 0.013353714 | 0.00000446 | 0.000708724 | 0.0000167 | 0.00209125 | 1 | 0.000146035 | 0.012922953 |
| ILMN_1237430 | 0.028815909 | 0.000240626 | 0.012267717 | 0.000354258 | 0.016129651 | 1 | 0.000844832 | 0.036512914 |
| ILMN_2723483 | 0.019410925 | 0.0000273 | 0.002563188 | 0.0000442 | 0.003965776 | 1 | 0.000228655 | 0.016840488 |
| ILMN_2958484 | 0.019410925 | 0.00000911 | 0.001168709 | 0.0000192 | 0.002327044 | 1 | 0.000157497 | 0.013500967 |
| ILMN_2661367 | 0.019410925 | 0.000367579 | 0.016499233 | 0.000301016 | 0.014549744 | 1 | 0.000306263 | 0.02048222 |
| ILMN_2608145 | 0.013353714 | 0.000037 | 0.003215513 | 0.000031 | 0.003134864 | 1 | 0.000228655 | 0.016840488 |
| ILMN_2791028 | 0.048451528 | 0.000126272 | 0.00779232 | 0.000471577 | 0.019546304 | 0 | 0.000540727 | 0.028504193 |
| ILMN_2524865 | 0.064412031 | 0.001199375 | 0.034848508 | 0.00116094 | 0.034266489 | 0 | 0.012346572 | 0.151955545 |
| ILMN_1254929 | 0.013353714 | 0.000000664 | 0.000160197 | 0.00000774 | 0.001225133 | 1 | 9.21504E−05 | 0.009952034 |
| ILMN_2939185 | 0.013353714 | 0.0000038 | 0.000630118 | 0.00000221 | 0.000472255 | 1 | 1.1878E−05 | 0.003141395 |
| ILMN_1228613 | 0.013353714 | 0.000141288 | 0.00855208 | 0.000044 | 0.003965776 | 1 | 5.74504E−05 | 0.007618639 |
| ILMN_2877507 | 0.019410925 | 0.0000468 | 0.003783616 | 0.000152378 | 0.009444363 | 1 | 0.000621233 | 0.03109305 |
| ILMN_2868131 | 0.028815909 | 0.0000929 | 0.006321664 | 0.000312052 | 0.014897503 | 1 | 0.0007895 | 0.035437666 |
| ILMN_1221943 | 0.039534447 | 0.000408571 | 0.017773612 | 0.000805135 | 0.027596907 | 0 | 0.001389407 | 0.047728966 |
| ILMN_2750515 | 0.013353714 | 0.0000144 | 0.001623511 | 0.000053 | 0.004480844 | 1 | 0.000643034 | 0.031573087 |
| ILMN_3005873 | 0.013353714 | 0.000000428 | 0.000117851 | 0.000000534 | 0.000169129 | 1 | 3.83949E−05 | 0.006099544 |
| ILMN_2934448 | 0.013353714 | 0.0000041 | 0.000665171 | 0.00000738 | 0.001188627 | 1 | 2.54447E−05 | 0.004756515 |
| ILMN_1240615 | 0.028815909 | 0.0000124 | 0.001448455 | 0.000029 | 0.003012161 | 1 | 0.000284828 | 0.019439747 |
| ILMN_2706231 | 0.042916604 | 0.00192157 | 0.047050677 | 0.001553237 | 0.040345167 | 1 | 0.00270263 | 0.068855671 |
| ILMN_2776850 | 0.019410925 | 0.0000167 | 0.001789771 | 0.0000788 | 0.005900268 | 1 | 0.000140602 | 0.012732723 |
| ILMN_2815889 | 0.013353714 | 0.0000165 | 0.001784328 | 0.0000203 | 0.002399815 | 1 | 3.99889E−05 | 0.006183935 |
| ILMN_1258394 | 0.013353714 | 1.52E−08 | 0.00000944 | 3.84E−08 | 0.0000267 | 1 | 2.99936E−06 | 0.00146959 |
| ILMN_2685565 | 0.013353714 | 0.000000595 | 0.000148947 | 0.00000154 | 0.000360893 | 1 | 6.47271E−05 | 0.008034278 |
| ILMN_1249313 | 0.013353714 | 0.00000837 | 0.001110274 | 0.0000181 | 0.002239201 | 1 | 8.19708E−05 | 0.009306714 |
| ILMN_2630459 | 0.109255622 | 0.001508456 | 0.040076536 | 0.00513661 | 0.082861032 | 0 | 0.009432496 | 0.132484733 |
| ILMN_2736690 | 0.013353714 | 0.00000361 | 0.000603853 | 0.000000336 | 0.00012714 | 1 | 1.64374E−06 | 0.001077622 |
| ILMN_2987863 | 0.028815909 | 0.000490467 | 0.0200248 | 0.000509451 | 0.020548879 | 1 | 0.001389407 | 0.047728966 |
| ILMN_2600744 | 0.013353714 | 0.0000862 | 0.005995631 | 0.0000228 | 0.002596277 | 1 | 5.09531E−05 | 0.007252947 |
| ILMN_3091641 | 0.056620496 | 0.000315614 | 0.014869318 | 0.000664858 | 0.024504648 | 0 | 0.003346676 | 0.077347433 |
| ILMN_2684651 | 0.024301667 | 0.000156156 | 0.009055643 | 0.000177574 | 0.010475755 | 1 | 0.00036664 | 0.02284602 |
| ILMN_2425415 | 0.013353714 | 0.000000227 | 0.0000712 | 0.000000367 | 0.000134456 | 1 | 2.65234E−05 | 0.004803829 |
| ILMN_2733733 | 0.032842408 | 0.0000662 | 0.004875361 | 0.000222439 | 0.012123944 | 1 | 0.000844832 | 0.036512914 |
| ILMN_2855261 | 0.013353714 | 0.000205243 | 0.011031683 | 0.000396081 | 0.017311138 | 1 | 0.00042277 | 0.024660011 |
| ILMN_2713464 | 0.013353714 | 0.0000249 | 0.002415508 | 0.000033 | 0.003242217 | 1 | 2.76455E−05 | 0.004930329 |
| ILMN_2574165 | 0.013353714 | 0.00000981 | 0.001218839 | 0.0000519 | 0.004442214 | 1 | 7.0054E−05 | 0.008469621 |
| ILMN_1259069 | 0.138695553 | 0.005671089 | 0.088200282 | 0.008881288 | 0.111410421 | 0 | 0.019156396 | 0.190243817 |
| ILMN_1252157 | 0.019410925 | 0.0000632 | 0.004700141 | 0.0000802 | 0.005962962 | 1 | 0.001179468 | 0.043782528 |
| ILMN_2935032 | 0.013353714 | 0.000000504 | 0.000131808 | 0.00000874 | 0.00131658 | 1 | 0.000120719 | 0.011491043 |
| ILMN_1253919 | 0.106524046 | 0.003457247 | 0.065871662 | 0.005703882 | 0.08745013 | 0 | 0.009432496 | 0.132484733 |
| ILMN_1260585 | 0.019410925 | 0.000174333 | 0.009713688 | 0.000297355 | 0.01443844 | 1 | 0.000816732 | 0.036034504 |
| ILMN_3136638 | 0.059256725 | 0.000405207 | 0.017710004 | 0.001023279 | 0.031817359 | 0 | 0.033458819 | 0.250266375 |
| ILMN_2936380 | 0.013353714 | 0.0000477 | 0.003816686 | 0.0000543 | 0.004539402 | 1 | 6.47271E−05 | 0.003034278 |
| ILMN_2673211 | 0.036357572 | 0.000674697 | 0.024612146 | 0.000839348 | 0.02829048 | 0 | 0.005858907 | 0.104408326 |
| ILMN_1221451 | 0.036357572 | 0.000515563 | 0.020723594 | 0.000820604 | 0.027941947 | 1 | 0.0007895 | 0.035437666 |
| ILMN_2450491 | 0.013353714 | 0.00000472 | 0.000733297 | 0.0000173 | 0.002159478 | 1 | 5.74504E−05 | 0.007618639 |
| ILMN_2790512 | 0.013353714 | 0.00000228 | 0.000426498 | 0.0000102 | 0.001443157 | 1 | 9.21504E−05 | 0.009952034 |
| ILMN_2894450 | 0.013353714 | 8.55E−08 | 0.0000344 | 0.000000606 | 0.000186894 | 1 | 1.89821E−05 | 0.004090548 |
| ILMN_1223966 | 0.032842408 | 0.002801542 | 0.059166678 | 0.001282138 | 0.036191446 | 0 | 0.001686537 | 0.053258626 |
| ILMN_2736347 | 0.013353714 | 1.05E−09 | 0.00000111 | 1.93E−09 | 0.00000357 | 1 | 2.60834E−07 | 0.000411872 |
| ILMN_2491099 | 0.013353714 | 0.00000196 | 0.00037186 | 0.00000128 | 0.000311058 | 1 | 3.43889E−06 | 0.001569314 |
| ILMN_1232396 | 0.303540704 | 0.050318123 | 0.288348039 | 0.060497533 | 0.307739355 | 0 | 0.057275046 | 0.323031815 |
| ILMN_1218602 | 0.036357572 | 0.000278859 | 0.01369203 | 0.000345319 | 0.015942739 | 0 | 0.001531347 | 0.050161589 |
| ILMN_3135781 | 0.019410925 | 0.0000259 | 0.002476668 | 0.0000866 | 0.006296133 | 1 | 0.000873828 | 0.037145252 |
| ILMN_2892376 | 0.013353714 | 0.000000949 | 0.000212379 | 0.00000258 | 0.000529628 | 1 | 2.384E−06 | 0.001305507 |
| ILMN_3161878 | 0.013353714 | 0.00000056 | 0.000143206 | 0.000000574 | 0.000179299 | 1 | 2.61375E−06 | 0.001367123 |
| ILMN_2847787 | 0.048451528 | 0.0000979 | 0.00654917 | 0.000701702 | 0.025381142 | 0 | 0.00388914 | 0.084081646 |
| ILMN_2646878 | 0.013353714 | 0.00000334 | 0.000570075 | 0.0000062 | 0.001053987 | 1 | 1.41013E−05 | 0.003473151 |
| ILMN_2706853 | 0.013353714 | 0.000000475 | 0.000127055 | 0.000000824 | 0.00022965 | 1 | 1.41024E−05 | 0.003473151 |
| ILMN_2843698 | 0.013353714 | 0.00000964 | 0.001212164 | 0.0000268 | 0.002862094 | 1 | 2.65234E−05 | 0.004803829 |
| ILMN_2717366 | 0.013353714 | 0.000130514 | 0.007988263 | 0.0000663 | 0.005180373 | 1 | 0.000317538 | 0.020788254 |
| ILMN_2546073 | 0.013353714 | 0.000000161 | 0.0000542 | 0.000000279 | 0.000110081 | 1 | 2.86522E−06 | 0.001449644 |
| ILMN_3128907 | 0.032842408 | 0.000542714 | 0.021366039 | 0.000678622 | 0.02485273 | 1 | 0.002387591 | 0.063870812 |
| ILMN_2678355 | 0.013353714 | 9.13E−08 | 0.000036 | 0.000000185 | 0.0000834 | 1 | 2.65193E−05 | 0.004803829 |
| ILMN_2655895 | 0.013353714 | 0.000000201 | 0.0000653 | 0.000000153 | 0.0000735 | 1 | 1.49656E−06 | 0.00102442 |
| ILMN_2625114 | 0.024301667 | 0.00000936 | 0.00118696 | 0.0000213 | 0.002477176 | 1 | 5.52021E−05 | 0.007602042 |
| ILMN_2667384 | 0.013353714 | 0.00000932 | 0.00118696 | 0.0000174 | 0.002159478 | 1 | 0.000246108 | 0.017705716 |
| ILMN_1222988 | 0.013353714 | 0.000586133 | 0.022426682 | 0.000199706 | 0.011313575 | 1 | 0.001104095 | 0.042298211 |
| ILMN_2895557 | 0.013353714 | 0.00000674 | 0.000954053 | 0.0000135 | 0.001778728 | 1 | 7.88278E−05 | 0.009015226 |
| ILMN_2925653 | 0.257087397 | 0.027419671 | 0.210973351 | 0.043010284 | 0.260513034 | 0 | 0.073688628 | 0.362080075 |
| ILMN_1216972 | 0.039534447 | 0.000148782 | 0.008799713 | 0.000332204 | 0.01550964 | 0 | 0.007560831 | 0.117979886 |
| ILMN_2652385 | 0.039534447 | 0.000862419 | 0.02851067 | 0.001249473 | 0.035680505 | 1 | 0.001482614 | 0.049612679 |
| ILMN_3158659 | 0.024301667 | 0.000220906 | 0.011671388 | 0.000139782 | 0.008999194 | 1 | 0.000966442 | 0.039356938 |
| ILMN_2675223 | 0.019410925 | 0.00001 | 0.001236109 | 0.0000453 | 0.004030508 | 1 | 0.000844832 | 0.036512914 |
| ILMN_2609073 | 0.013353714 | 0.0000179 | 0.001868062 | 0.000136367 | 0.008839388 | 1 | 0.000204627 | 0.01592867 |
| ILMN_2588055 | 0.654115083 | 0.24448523 | 0.598658009 | 0.320992437 | 0.661760783 | 0 | 0.345393797 | 0.694142958 |
| ILMN_2680686 | 0.013353714 | 0.0000444 | 0.003643784 | 0.0000134 | 0.001768921 | 1 | 1.29447E−05 | 0.003328934 |
| ILMN_1219820 | 0.019410925 | 0.00000983 | 0.001218839 | 0.0000795 | 0.0059192 | 1 | 9.95836E−05 | 0.010463471 |
| ILMN_2589893 | 0.019410925 | 0.000177516 | 0.009836693 | 0.000246793 | 0.012817366 | 1 | 0.00036664 | 0.02284602 |
| ILMN_2975312 | 0.039534447 | 0.000293646 | 0.014134784 | 0.00040153 | 0.017418469 | 1 | 0.002243019 | 0.061925158 |
| ILMN_2667033 | 0.013353714 | 0.00000237 | 0.000439395 | 0.0000082 | 0.00127918 | 1 | 2.34111E−05 | 0.00450296 |
| ILMN_2622671 | 0.013353714 | 0.000000366 | 0.000104618 | 0.000000801 | 0.000227328 | 1 | 9.15833E−06 | 0.002681086 |
| ILMN_2674324 | 0.019410925 | 0.0000524 | 0.004058867 | 0.000133735 | 0.008742949 | 1 | 0.000486846 | 0.026691654 |
| ILMN_1259290 | 0.013353714 | 0.000000064 | 0.0000271 | 0.000000238 | 0.000101599 | 1 | 3.28593E−06 | 0.001544952 |
| ILMN_2932539 | 0.013353714 | 0.000000686 | 0.000164623 | 0.000000768 | 0.000219738 | 1 | 2.15328E−05 | 0.004320203 |
| ILMN_1253819 | 0.013353714 | 2.15E−08 | 0.0000125 | 0.000000103 | 0.0000554 | 1 | 5.39498E−06 | 0.002025163 |
| ILMN_2995688 | 0.145558421 | 0.006861309 | 0.097821135 | 0.010110344 | 0.119739224 | 0 | 0.023367993 | 0.210226124 |
| ILMN_1238640 | 0.019410925 | 0.0000179 | 0.001868062 | 0.0000445 | 0.003979315 | 1 | 0.000264806 | 0.018619193 |
| ILMN_2696696 | 0.032842408 | 0.0000495 | 0.003917954 | 0.000354841 | 0.016129651 | 0 | 0.00270263 | 0.068855671 |
| ILMN_1225192 | 0.013353714 | 3.36E−10 | 0.000000504 | 1.02E−09 | 0.00000206 | 1 | 6.54696E−08 | 0.000253951 |
| ILMN_1243066 | 0.13387405 | 0.006676758 | 0.096576461 | 0.007146886 | 0.098567729 | 0 | 0.046032778 | 0.290967914 |
| ILMN_2937320 | 0.013353714 | 0.0000122 | 0.001439713 | 0.0000348 | 0.003370933 | 1 | 0.000176289 | 0.014446733 |
| ILMN_2731237 | 0.013353714 | 0.0000214 | 0.002161787 | 0.0000762 | 0.005756583 | 1 | 0.00040803 | 0.024132893 |
| ILMN_2599657 | 0.013353714 | 0.00000477 | 0.000734844 | 0.00000943 | 0.00136995 | 1 | 1.81963E−05 | 0.003995524 |
| ILMN_2731550 | 0.013353714 | 0.00000353 | 0.000593169 | 0.00000482 | 0.000858959 | 1 | 6.47271E−05 | 0.008034278 |
| ILMN_2762189 | 0.013353714 | 3.36E−08 | 0.0000172 | 0.000000756 | 0.000219513 | 1 | 9.99092E−06 | 0.002818469 |
| ILMN_2959291 | 0.024301667 | 0.000416306 | 0.01805945 | 0.000280609 | 0.013898949 | 1 | 0.001301631 | 0.046179135 |
| ILMN_2841289 | 0.013353714 | 0.00000721 | 0.000992417 | 0.00000322 | 0.00064069 | 1 | 3.26018E−05 | 0.005579105 |
| ILMN_2671411 | 0.013353714 | 5.07E−08 | 0.0000227 | 0.000000361 | 0.00013352 | 1 | 5.15939E−06 | 0.002018103 |
| ILMN_2596522 | 0.013353714 | 0.00000371 | 0.000617087 | 0.000000442 | 0.000148085 | 1 | 1.41024E−05 | 0.003473151 |
| ILMN_2939681 | 0.066594341 | 0.001271844 | 0.036075887 | 0.001559402 | 0.040474884 | 0 | 0.004644853 | 0.093111105 |
| ILMN_2964076 | 0.024301667 | 0.000121309 | 0.007605138 | 0.000192578 | 0.011039321 | 1 | 0.000504223 | 0.027322568 |
| ILMN_2692797 | 0.013353714 | 0.0000212 | 0.002151254 | 0.0000583 | 0.004702444 | 1 | 0.001259689 | 0.045559243 |
| ILMN_2693124 | 0.117507061 | 0.003534836 | 0.066748886 | 0.005131646 | 0.08285783 | 0 | 0.015623977 | 0.17119804 |
| ILMN_1220769 | 0.013353714 | 1.01E−08 | 0.00000691 | 8.75E−05 | 0.0000485 | 1 | 5.74828E−07 | 0.000569287 |
| ILMN_2701271 | 0.013353714 | 0.000000115 | 0.0000431 | 5.41E−08 | 0.0000344 | 1 | 5.21457E−07 | 0.000539384 |
| ILMN_2778289 | 0.132649005 | 0.002207096 | 0.051341175 | 0.006003207 | 0.089676275 | 0 | 0.041147128 | 0.276939586 |
| ILMN_1249362 | 0.028815909 | 0.000225617 | 0.011813028 | 0.000591023 | 0.022623651 | 1 | 0.001141234 | 0.042874093 |
| ILMN_2661287 | 0.013353714 | 0.000000401 | 0.000112548 | 0.000000715 | 0.000216027 | 1 | 3.59833E−06 | 0.001580255 |
| ILMN_2846865 | 0.617844208 | 0.203970444 | 0.553566103 | 0.275551624 | 0.619558566 | 0 | 0.257914169 | 0.618884979 |
| ILMN_2925711 | 0.013353714 | 0.000000985 | 0.000217336 | 0.0000039 | 0.000746457 | 1 | 4.70154E−05 | 0.006881837 |
| ILMN_2803674 | 0.024301667 | 0.000323505 | 0.015111037 | 0.000191432 | 0.01098713 | 1 | 0.001633268 | 0.052249986 |
| ILMN_2866276 | 0.013353714 | 0.0000165 | 0.001784328 | 0.0000775 | 0.005835203 | 1 | 0.00040803 | 0.024132893 |
| ILMN_2524986 | 0.156283296 | 0.015615825 | 0.157365186 | 0.022122415 | 0.183826235 | 0 | 0.019643695 | 0.192333843 |
| ILMN_2479717 | 0.013353714 | 0.00000201 | 0.000379002 | 0.00000853 | 0.001297207 | 1 | 2.65234E−05 | 0.004803829 |
| ILMN_2987709 | 0.013353714 | 0.0000166 | 0.001784328 | 0.0000144 | 0.001881854 | 1 | 0.000212368 | 0.016047225 |
| ILMN_1224736 | 0.013353714 | 0.0000174 | 0.001836085 | 0.0000098 | 0.001399019 | 1 | 2.34111E−05 | 0.00450296 |
| ILMN_3155815 | 0.013353714 | 0.000000235 | 0.0000718 | 0.00000169 | 0.000383182 | 1 | 6.16514E−06 | 0.002125696 |
| ILMN_2654754 | 0.076777595 | 0.005066053 | 0.082537476 | 0.003571366 | 0.067411346 | 0 | 0.016875145 | 0.178845026 |
| ILMN_3104139 | 0.013353714 | 1.43E−08 | 0.00000901 | 1.38E−08 | 0.0000126 | 1 | 1.15863E−07 | 0.000299615 |
| ILMN_2712986 | 0.032842408 | 0.000875122 | 0.028689223 | 0.000739816 | 0.026207168 | 1 | 0.004127635 | 0.087093854 |
| ILMN_2757368 | 0.013353714 | 2.3E−09 | 0.00000185 | 1.18E−08 | 0.0000119 | 1 | 2.74304E−07 | 0.000411872 |
| ILMN_2491202 | 0.013353714 | 4.51E−09 | 0.00000339 | 8.56E−09 | 0.0000105 | 1 | 2.24455E−07 | 0.000373132 |
| ILMN_1230766 | 0.013353714 | 4.83E−08 | 0.0000223 | 0.000000186 | 0.0000834 | 1 | 4.93323E−06 | 0.001962625 |
| ILMN_2771380 | 0.094076848 | 0.002100066 | 0.049831197 | 0.006368661 | 0.092696085 | 0 | 0.009693098 | 0.134605773 |
| ILMN_1230708 | 0.013353714 | 0.000000172 | 0.0000571 | 0.00000112 | 0.000290018 | 1 | 1.53585E−05 | 0.003592421 |
| ILMN_2684575 | 0.019410925 | 0.0000565 | 0.004316317 | 0.000142325 | 0.009037925 | 1 | 0.000438006 | 0.025077344 |
| ILMN_1218347 | 0.013353714 | 1.44E−09 | 0.00000136 | 5.7E−09 | 0.00000758 | 1 | 3.34766E−07 | 0.00044521 |
| ILMN_2601946 | 0.013353714 | 0.00000259 | 0.000464931 | 0.00000166 | 0.000379819 | 1 | 0.000317538 | 0.020788254 |
| ILMN_2835117 | 0.013353714 | 0.000000799 | 0.000185854 | 0.00000889 | 0.001330104 | 1 | 9.5799E−05 | 0.010180718 |
| ILMN_2759484 | 0.013353714 | 0.0000685 | 0.005001847 | 0.0000487 | 0.004231895 | 1 | 0.000438006 | 0.025077344 |
| ILMN_1223416 | 0.013353714 | 4.78E−08 | 0.0000223 | 0.000000394 | 0.00013797 | 1 | 3.99889E−05 | 0.006183935 |
| ILMN_2901626 | 0.019410925 | 0.00000168 | 0.000330869 | 0.0000142 | 0.001859837 | 1 | 0.00010758 | 0.010838827 |
| ILMN_2693895 | 0.013353714 | 0.00000119 | 0.000254149 | 0.00000516 | 0.00090936 | 1 | 0.000151664 | 0.013244851 |
| ILMN_2777498 | 0.064412031 | 0.001354766 | 0.037413073 | 0.001755499 | 0.043416704 | 0 | 0.004379328 | 0.090517125 |
| ILMN_2789900 | 0.013353714 | 0.00000234 | 0.000436452 | 0.00000186 | 0.000412865 | 1 | 1.89821E−05 | 0.004090548 |
| ILMN_2618423 | 0.013353714 | 9.02E−10 | 0.000000976 | 0.000000002 | 0.00000357 | 1 | 1.04496E−07 | 0.000299615 |
| ILMN_1242457 | 0.039534447 | 0.000122008 | 0.007605138 | 0.000177435 | 0.010475755 | 0 | 0.00093456 | 0.038635069 |
| ILMN_2742075 | 0.046026289 | 0.000386811 | 0.017128258 | 0.000499048 | 0.020287507 | 0 | 0.001389407 | 0.047728966 |
| ILMN_2868220 | 0.024301667 | 0.000165236 | 0.009379559 | 0.000240793 | 0.012650343 | 1 | 0.000470028 | 0.02607676 |
| ILMN_2674884 | 0.024301567 | 0.0000257 | 0.002473139 | 0.0000641 | 0.00508011 | 1 | 0.000438006 | 0.025077344 |
| ILMN_3161601 | 0.032842408 | 0.00000758 | 0.001035225 | 0.0000792 | 0.005917903 | 1 | 0.007351981 | 0.116676672 |
| ILMN_2705860 | 0.019410925 | 0.000013 | 0.00149316 | 0.00006 | 0.004813511 | 1 | 0.000934606 | 0.038635069 |
| ILMN_2710905 | 0.013353714 | 0.0000119 | 0.001426193 | 0.0000147 | 0.001907372 | 1 | 0.000182986 | 0.014712435 |
| ILMN_2595732 | 0.013353714 | 0.000001 | 0.000219086 | 0.000000217 | 0.0000944 | 1 | 5.15939E−06 | 0.002018103 |
| ILMN_2719732 | 0.013353714 | 0.00000901 | 0.001164448 | 0.0000212 | 0.002477176 | 1 | 0.000540727 | 0.028504193 |
| ILMN_1221700 | 0.013353714 | 3.27E−09 | 0.00000253 | 1.67E−09 | 0.00000324 | 1 | 2.02641E−08 | 0.000188647 |
| ILMN_1224472 | 0.019410925 | 0.00000658 | 0.000945204 | 0.0000352 | 0.003396141 | 1 | 0.000255296 | 0.018170157 |
| ILMN_2728038 | 0.013353714 | 4.47E−11 | 8.33E−08 | 1.1E−10 | 0.000000395 | 1 | 6.03547E−07 | 0.000573333 |
| ILMN_1252819 | 0.013353714 | 8.95E−10 | 0.000000976 | 7.15E−10 | 0.00000159 | 1 | 4.07147E−08 | 0.000249449 |
| ILMN_2538531 | 0.013353714 | 0.00012104 | 0.007605138 | 0.000511029 | 0.020559076 | 1 | 0.001104095 | 0.042298211 |
| ILMN_2493826 | 0.013353714 | 1.33E−08 | 0.0000087 | 8.14E−08 | 0.0000468 | 1 | 3.13951E−06 | 0.001522236 |
| ILMN_2834379 | 0.028815909 | 0.000937725 | 0.030098764 | 0.000249034 | 0.012894089 | 1 | 0.001068077 | 0.041672924 |
| ILMN_1222543 | 0.013353714 | 1.46E−09 | 0.00000136 | 4.37E−08 | 0.0000295 | 1 | 3.59866E−06 | 0.001580255 |
| ILMN_2983516 | 0.019410925 | 0.0000123 | 0.001442533 | 0.0000539 | 0.004524583 | 1 | 0.000643034 | 0.031573087 |
| ILMN_2426480 | 0.013353714 | 7.32E−08 | 0.0000304 | 0.00000104 | 0.000275608 | 1 | 9.21504E−05 | 0.009952034 |
| ILMN_2592554 | 0.013353714 | 2.19E−09 | 0.00000179 | 0.000000029 | 0.0000218 | 1 | 7.3257E−07 | 0.000681979 |
| ILMN_1220548 | 0.013353714 | 2.09E−08 | 0.0000125 | 0.000000374 | 0.000136 | 1 | 9.99092E−06 | 0.002818469 |
| ILMN_1233336 | 0.019410925 | 0.0000163 | 0.001773716 | 0.0000255 | 0.00279613 | 1 | 0.00040803 | 0.024132893 |
| ILMN_2646985 | 0.013353714 | 0.000000122 | 0.0000454 | 0.00000123 | 0.000301817 | 1 | 2.15311E−05 | 0.004320203 |
| ILMN_2925094 | 0.013353714 | 0.00000078 | 0.0001843 | 0.00000115 | 0.000292494 | 1 | 1.89821E−05 | 0.004090548 |
| ILMN_1252076 | 0.032842408 | 0.0000728 | 0.005280083 | 0.000328357 | 0.015438431 | 0 | 0.002314272 | 0.062958735 |
| ILMN_2948143 | 0.013353714 | 0.000000544 | 0.000140657 | 0.00000159 | 0.000369569 | 1 | 0.000120719 | 0.011491043 |
| ILMN_1219230 | 0.013353714 | 0.00000132 | 0.000271671 | 0.00000103 | 0.000274668 | 1 | 2.15328E−05 | 0.004320203 |
| ILMN_1230140 | 0.013353714 | 0.00000304 | 0.000527536 | 0.0000057 | 0.000979318 | 1 | 3.53859E−05 | 0.005861598 |
| ILMN_3162125 | 0.013353714 | 0.000000208 | 0.0000667 | 0.000000012 | 0.0000119 | 1 | 4.9657E−07 | 0.000525315 |
| ILMN_2771176 | 0.013353714 | 1.02E−11 | 0.000000025 | 4.19E−10 | 0.000000976 | 1 | 4.07997E−07 | 0.000499764 |
| ILMN_2847115 | 0.019410925 | 0.00000482 | 0.000740299 | 0.0000154 | 0.001964139 | 1 | 0.00011618 | 0.011242898 |
| ILMN_2758029 | 0.013353714 | 1.53E−10 | 0.000000264 | 3.43E−10 | 0.00000084 | 1 | 5.59305E−08 | 0.000249449 |
| ILMN_2722996 | 0.013353714 | 1.66E−09 | 0.00000149 | 3.18E−09 | 0.00000462 | 1 | 3.28593E−06 | 0.001544952 |
| ILMN_2657828 | 0.013353714 | 2.44E−12 | 9.46E−09 | 1.36E−10 | 0.000000429 | 1 | 5.89499E−08 | 0.000249449 |
| ILMN_2712075 | 0.013353714 | 0.00000262 | 0.000466947 | 0.00000288 | 0.000577427 | 1 | 2.44059E−05 | 0.00463717 |
| ILMN_2878071 | 0.013353714 | 5.41E−08 | 0.000024 | 0.00000139 | 0.000336099 | 1 | 7.57938E−05 | 0.008797941 |
| ILMN_2705628 | 0.019410925 | 0.00000062 | 0.000153621 | 0.0000039 | 0.000746457 | 1 | 7.0054E−05 | 0.008469621 |
| ILMN_1233982 | 0.013353714 | 0.00000694 | 0.000978706 | 0.00000394 | 0.000746457 | 1 | 4.51564E−05 | 0.006758509 |
| ILMN_1377923 | 0.269665294 | 0.009183237 | 0.115903512 | 0.030148726 | 0.215797746 | 0 | 0.014835081 | 0.166562185 |
| ILMN_1249030 | 0.013353714 | 0.0000017 | 0.000334725 | 0.0000195 | 0.002344998 | 1 | 0.00022037 | 0.016464793 |
| ILMN_2538242 | 0.013353714 | 0.000015 | 0.001669081 | 0.00O0107 | 0.001483462 | 1 | 0.000135361 | 0.012427306 |
| ILMN_2993314 | 0.013353714 | 0.00000115 | 0.00024883 | 0.000015 | 0.001929689 | 1 | 0.000163541 | 0.013740705 |
| ILMN_2750842 | 0.013353714 | 0.00000254 | 0.00046434 | 0.00000545 | 0.000950899 | 1 | 6.44447E−06 | 0.002189569 |
| ILMN_2600421 | 0.013353714 | 8.09E−10 | 0.000000934 | 2.37E−08 | 0.0000187 | 1 | 8.76731E−06 | 0.002615973 |
| ILMN_2763245 | 0.013353714 | 0.000000237 | 0.0000718 | 0.00000181 | 0.000406 | 1 | 9.95836E−05 | 0.010463471 |
| ILMN_1253874 | 0.013353714 | 6.55E−10 | 0.000000824 | 9.01E−09 | 0.0000105 | 1 | 2.38377E−06 | 0.001305507 |
| ILMN_1215076 | 0.028815909 | 0.00115136 | 0.03420847 | 0.000886 | 0.0292 | 1 | 0.001344859 | 0.046961097 |
| ILMN_2725259 | 0.013353714 | 5.55E−14 | 6.46E−10 | 1.82E−11 | 0.000000121 | 1 | 9.40233E−09 | 0.000122224 |

Because both FOXP3 and GPR83 are associated with Tregs (47) and both have more consistent and significantly higher expression induced by GA than by PG, we sought to determine on a persample basis whether the same subset of PG samples induced low levels of both genes, or whether FOXP3 was low in some PG samples and GPR83 was low in other PG samples. We confirmed that the same PG samples that were low in FOXP3 were also low in GPR83 (FIG. 6C).

When the genes differentially expressed in response to different medicines are also transcription factors (e.g. FOXP3), we can further test the observation by examining the expression of genes known to be targets of that transcription factor. In this case, we sought to determine whether genes downstream of FoxP3 are upregulated following activation by GA as compared to PG.

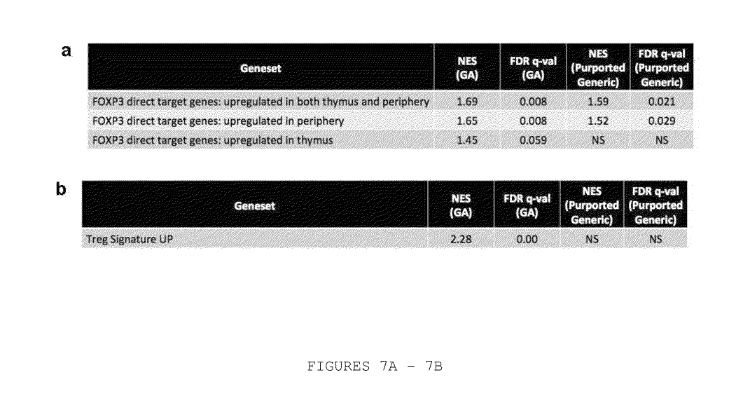

Through Gene Set Enrichment Analysis (GSEA), (48) we found that FoxP3 targets genes were enriched in GA samples compared to medium (FDR-adjusted q=0.008) to a more significant degree than in PG samples compared to medium (FDR-adjusted q=0.036, FIG. 7A).

For the list of genes with significantly higher expression induced by GA than PG by the nonparametric Wilcoxon rank-sum test, we utilized the Molecular Signature Database (MSigDB) (48) to determine that the same list of genes bound by FoxP3 was significantly enriched (adjusted p<1.59×10−8, Table 9).

TABLE 9
MSigDB enrichment results for the list of genes with
significantly different expression between GA and other glatiramoid
by the Wilcoxon rank sum test, including FoxP3 targets among the
enriched signatures for genes higher in GA than other glatiramoid,
and TLR and LPS pathways among the enriched signatures for genes
higher in other glatiramoid than GA.

| Reference Set | User Set | N | K | n | k | P | BHP_P |
|---|---|---|---|---|---|---|---|
| BLALOCK_ALZHEIMERS_DISEASE_UP | wilcox_up_in_GA | 31847 | 1691 | 392 | 54 | 0 | 0 |
| PILON_KLF1_TARGETS_DN | wilcox_up_in_GA | 31847 | 1972 | 392 | 80 | 0 | 0 |
| MEMBRANE | wilcox_up_in_GA | 31847 | 1994 | 392 | 65 | 0 | 0 |
| GRAESSMANN_APOPTOSIS_BY_DOXORUBICIN_DN | wilcox_up_in_GA | 31847 | 1781 | 392 | 60 | 0 | 0 |
| GGGAGGRR_V$MAZ_Q6 | wilcox_up_in_GA | 31847 | 2274 | 392 | 69 | 0 | 0 |
| SIGNAL_TRANSDUCTION | wilcox_up_in_GA | 31847 | 1634 | 392 | 60 | 0 | 0 |
| TTGTTT_V$FOXO4_01 | wilcox_up_in_GA | 31847 | 2061 | 392 | 64 | 0 | 0 |
| DUTERTRE_ESTRADIOL_RESPONSE_24HR_DN | wilcox_up_in_GA | 31847 | 505 | 392 | 30 | 0 | 0 |
| GNF2_MATK | wilcox_up_in_GA | 31847 | 25 | 392 | 10 | 0 | 0 |
| PID_TCR_PATHWAY | wilcox_up_in_GA | 31847 | 66 | 392 | 14 | 3.55E−12 | 8.55E−10 |
| MEMBRANE_PART | wilcox_up_in_GA | 31847 | 1670 | 392 | 53 | 8.99E−12 | 1.97E−09 |
| BIOPOLYMER_METABOLIC_PROCESS | wilcox_up_in_GA | 31847 | 1684 | 392 | 53 | 1.10E−11 | 2.20E−09 |
| PID_CD8TCRDOWNSTREAMPATHWAY | wilcox_up_in_GA | 31847 | 65 | 392 | 12 | 4.02E−11 | 7.45E−09 |
| REACTOME_TCR_SIGNALING | wilcox_up_in_GA | 31847 | 54 | 392 | 13 | 4.93E−11 | 8.47E−09 |
| PID_CD8TCRPATHWAY | wilcox_up_in_GA | 31847 | 53 | 392 | 12 | 7.51E−11 | 1.21E−08 |
| SMID_BREAST_CANCER_NORMAL_LIKE_UP | wilcox_up_in_GA | 31847 | 476 | 392 | 48 | 9.86E−11 | 1.44E−08 |
| PUJANA_ATM_PCC_NETWORK | wilcox_up_in_GA | 31847 | 1442 | 392 | 52 | 1.02E−10 | 1.44E−08 |
| GNF2_PTPN4 | wilcox_up_in_GA | 31847 | 51 | 392 | 11 | 1.17E−10 | 1.57E−08 |
| MODULE_64 | wilcox_up_in_GA | 31847 | 518 | 392 | 28 | 1.30E−10 | 1.59E−08 |
| PID_IL12_STAT4PATHWAY | wilcox_up_in_GA | 31847 | 33 | 392 | 9 | 1.37E−10 | 1.59E−08 |
| ZHENG_BOUND_BY_FOXP3 | wilcox_up_in_GA | 31847 | 491 | 392 | 56 | 1.42E−10 | 1.59E−08 |
| DIAZ_CHRONIC_MEYLOGENOUS_LEUKEMIA_UP | wilcox_up_in_GA | 31847 | 1382 | 392 | 51 | 1.46E−10 | 1.59E−08 |
| CHEN_METABOLIC_SYNDROM_NETWORK | wilcox_up_in_GA | 31847 | 1210 | 392 | 46 | 1.72E−10 | 1.79E−08 |
| MODULE_75 | wilcox_up_in_GA | 31847 | 399 | 392 | 29 | 1.86E−10 | 1.80E−08 |
| MODULE_84 | wilcox_up_in_GA | 31847 | 549 | 392 | 38 | 1.87E−10 | 1.80E−08 |
| MARSON_BOUND_BY_FOXP3_UNSTIMULATED | wilcox_up_in_GA | 31847 | 1229 | 392 | 66 | 2.04E−10 | 1.80E−08 |
| LINDGREN_BLADDER_CANCER_CLUSTER_2B | wilcox_up_in_GA | 31847 | 392 | 392 | 29 | 2.12E−10 | 1.80E−08 |
| SANSOM_APC_TARGETS_DN | wilcox_up_in_GA | 31847 | 366 | 392 | 25 | 2.16E−10 | 1.80E−08 |
| MODULE_46 | wilcox_up_in_GA | 31847 | 395 | 392 | 29 | 2.27E−10 | 1.80E−08 |
| SMID_BREAST_CANCER_LUMINAL_B_DN | wilcox_up_in_GA | 31847 | 564 | 392 | 30 | 2.32E−10 | 1.80E−08 |
| GCACTTT, MIR-17-5P, MIR- | wilcox_up_in_GA | 31847 | 595 | 392 | 33 | 2.46E−10 | 1.80E−08 |
| 20A, MIR-106A, MIR- | | | | | | | |
| 106B, MIR-20B, MIR-519D | | | | | | | |
| NUYTTEN_EZH2_TARGETS_UP | wilcox_up_in_GA | 31847 | 1037 | 392 | 44 | 2.46E−10 | 1.80E−08 |
| KUMAR_TARGETS_OF_MLL_AF9_FUSION | wilcox_up_in_GA | 31847 | 405 | 392 | 26 | 2.47E−10 | 1.80E−08 |
| MARSON_BOUND_BY_FOXP3_STIMULATED | wilcox_up_in_GA | 31847 | 1022 | 392 | 54 | 2.65E−10 | 1.83E−08 |
| GOBERT_OLIGODENDROCYTE_DIFFERENTIATION_DN | wilcox_up_in_GA | 31847 | 1080 | 392 | 44 | 2.67E−10 | 1.83E−08 |
| REACTOME_IMMUNE_SYSTEM | wilcox_up_in_GA | 31847 | 933 | 392 | 51 | 2.82E−10 | 1.88E−08 |
| MODULE_45 | wilcox_up_in_GA | 31847 | 583 | 392 | 33 | 2.99E−10 | 1.95E−08 |
| CYTOPLASM | wilcox_up_in_generic | 31847 | 2131 | 439 | 68 | 0 | 0 |
| BLALOCK_ALZHEIMERS_DISEASE_UP | wilcox_up_in_generic | 31847 | 1691 | 439 | 59 | 0 | 0 |
| PUJANA_BRCA1_PCC_NETWORK | wilcox_up_in_generic | 31847 | 1652 | 439 | 57 | 0 | 0 |
| PILON_KLF1_TARGETS_DN | wilcox_up_in_generic | 31847 | 1972 | 439 | 81 | 0 | 0 |
| GRAESSMANN_APOPTOSIS_BY_DOXORUBICIN_DN | wilcox_up_in_generic | 31847 | 1781 | 439 | 68 | 0 | 0 |
| DIAZ_CHRONIC_MEYLOGENOUS_LEUKEMIA_UP | wilcox_up_in_generic | 31847 | 1382 | 439 | 56 | 6.56E−11 | 2.51E−08 |
| KRIGE_RESPONSE_TO_TOSEDOSTAT_6HR_UP | wilcox_up_in_generic | 31847 | 953 | 439 | 46 | 1.20E−10 | 3.57E−08 |
| KRIGE_RESPONSE_TO_TOSEDOSTAT_24HR_DN | wilcox_up_in_generic | 31847 | 1011 | 439 | 51 | 1.24E−10 | 3.57E−08 |
| GRAESSMANN_APOPTOSIS_BY_DOXORUBICIN_UP | wilcox_up_in_generic | 31847 | 1142 | 439 | 46 | 2.24E−10 | 5.64E−08 |
| REACTOME_IMMUNE_SYSTEM | wilcox_up_in_generic | 31847 | 933 | 439 | 41 | 2.48E−10 | 5.64E−08 |
| KRIGE_RESPONSE_TO_TOSEDOSTAT_6HR_DN | wilcox_up_in_generic | 31847 | 911 | 439 | 45 | 2.74E−10 | 5.64E−08 |
| CHEN_METABOLIC_SYNDROM_NETWORK | wilcox_up_in_generic | 31847 | 1210 | 439 | 48 | 2.95E−10 | 5.64E−08 |
| PROTEIN_METABOLIC_PROCESS | wilcox_up_in_generic | 31847 | 1231 | 439 | 48 | 3.86E−10 | 6.58E−08 |
| LEE_BMP2_TARGETS_DN | wilcox_up_in_generic | 31847 | 882 | 439 | 52 | 4.01E−10 | 6.58E−08 |
| WEI_MYCN_TARGETS_WITH_E_BOX | wilcox_up_in_generic | 31847 | 795 | 439 | 39 | 5.50E−10 | 8.42E−08 |
| MODULE_5 | wilcox_up_in_generic | 31847 | 434 | 439 | 26 | 6.51E−10 | 9.34E−08 |
| GGGCGGR_V$SP1_Q6 | wilcox_up_in_generic | 31847 | 2940 | 439 | 95 | 7.24E−10 | 9.78E−08 |
| BYSTRYKH_HEMATOPOIESIS_STEM_CELL_QTL_TRANS | wilcox_up_in_generic | 31847 | 882 | 439 | 38 | 1.16E−09 | 1.48E−07 |
| ENK_UV_RESPONSE_EPIDERMIS_UP | wilcox_up_in_generic | 31847 | 293 | 439 | 21 | 1.31E−09 | 1.59E−07 |
| MONNIER_POSTRADIATION_TUMOR_ESCAPE_UP | wilcox_up_in_generic | 31847 | 393 | 439 | 24 | 1.69E−09 | 1.94E−07 |
| BROWN_MYELOID_CELL_DEVELOPMENT_UP | wilcox_up_in_generic | 31847 | 165 | 439 | 16 | 1.91E−09 | 2.09E−07 |
| SENESE_HDAC3_TARGETS_UP | wilcox_up_in_generic | 31847 | 501 | 439 | 27 | 2.47E−09 | 2.58E−07 |
| BIOPOLYMER_METABOLIC_PROCESS | wilcox_up_in_generic | 31847 | 1684 | 439 | 55 | 2.71E−09 | 2.71E−07 |
| GNF2_CARD15 | wilcox_up_in_generic | 31847 | 70 | 439 | 11 | 3.41E−09 | 3.26E−07 |
| DODD_NASOPHARYNGEAL_CARCINOMA_DN | wilcox_up_in_generic | 31847 | 1375 | 439 | 48 | 4.41E−09 | 4.05E−07 |
| SCGGAAGY_V$ELK1_02 | wilcox_up_in_generic | 31847 | 1199 | 439 | 44 | 4.79E−09 | 4.23E−07 |
| VERHAAK_AML_WITH_NPM1_MUTATED_UP | wilcox_up_in_generic | 31847 | 183 | 439 | 16 | 6.55E−09 | 5.57E−07 |
| CAIRO_HEPATOBLASTOMA_CLASSES_UP | wilcox_up_in_generic | 31847 | 605 | 439 | 29 | 8.42E−09 | 6.91E−07 |
| GNF2_CD1D | wilcox_up_in_generic | 31847 | 45 | 439 | 9 | 9.51E−09 | 7.53E−07 |
| RUTELLA_RESPONSE_TO_CSF2RB_AND_IL4_UP | wilcox_up_in_generic | 31847 | 338 | 439 | 21 | 1.29E−08 | 9.69E−07 |
| LI_INDUCED_T_TO_NATURAL_KILLER_UP | wilcox_up_in_generic | 31847 | 307 | 439 | 20 | 1.31E−08 | 9.69E−07 |
| BOYLAN_MULTIPLE_MYELOMA_C_D_DN | wilcox_up_in_generic | 31847 | 252 | 439 | 18 | 1.73E−08 | 1.24E−06 |
| KEGG_TOLL_LIKE_RECEPTOR_SIGNALING_PATHWAY | wilcox_up_in_generic | 31847 | 102 | 439 | 12 | 1.83E−08 | 1.27E−06 |
| MODULE_6 | wilcox_up_in_generic | 31847 | 416 | 439 | 23 | 2.30E−08 | 1.55E−06 |
| RUTELLA_RESPONSE_TO_HGF_UP | wilcox_up_in_generic | 31847 | 418 | 439 | 23 | 2.51E−08 | 1.65E−06 |
| MODULE_3 | wilcox_up_in_generic | 31847 | 385 | 439 | 22 | 2.59E−08 | 1.65E−06 |
| CHARAFE_BREAST_CANCER_LUMINAL_VS_BASAL_DN | wilcox_up_in_generic | 31847 | 455 | 439 | 24 | 2.78E−08 | 1.73E−06 |
| MARTENS_BOUND_BY_PML_RARA_FUSION | wilcox_up_in_generic | 31847 | 456 | 439 | 24 | 2.90E−08 | 1.75E−06 |
| GNF2_CD33 | wilcox_up_in_generic | 31847 | 52 | 439 | 9 | 3.62E−08 | 2.13E−06 |
| NUCLEOBASENUCLEOSIDENUCLEOTIDE_AND_NUCLEIC_{—} | wilcox_up_in_generic | 31847 | 1244 | 439 | 43 | 3.88E−08 | 2.23E−06 |
| ACID_METABOLIC_PROCESS | | | | | | | |
| TONKS_TARGETS_OF_RUNX1_RUNX1T1_FUSION_{—} | wilcox_up_in_generic | 31847 | 157 | 439 | 14 | 4.29E−08 | 2.41E−06 |
| ERYTHROCYTE_UP | | | | | | | |
| FOSTER_KDM1A_TARGETS_DN | wilcox_up_in_generic | 31847 | 211 | 439 | 16 | 4.64E−08 | 2.54E−06 |
| NUYTTEN_EZH2_TARGETS_UP | wilcox_up_in_generic | 31847 | 1037 | 439 | 38 | 5.75E−08 | 3.05E−06 |
| MARKEY_RB1_ACUTE_LOF_UP | wilcox_up_in_generic | 31847 | 215 | 439 | 16 | 6.02E−08 | 3.05E−06 |
| CASORELLI_ACUTE_PROMYELOCYTIC_LEUKEMIA_DN | wilcox_up_in_generic | 31847 | 663 | 439 | 29 | 6.04E−08 | 3.05E−06 |
| MODULE_16 | wilcox_up_in_generic | 31847 | 511 | 439 | 25 | 6.11E−08 | 3.05E−06 |
| BRUINS_UVC_RESPONSE_LATE | wilcox_up_in_generic | 31847 | 1137 | 439 | 40 | 7.46E−08 | 3.65E−06 |
| ZHANG_TLX_TARGETS_36HR_UP | wilcox_up_in_generic | 31847 | 221 | 439 | 16 | 8.79E−08 | 4.21E−06 |
| RODWELL_AGING_KIDNEY_UP | wilcox_up_in_generic | 31847 | 487 | 439 | 24 | 9.84E−08 | 4.61E−06 |
| SEKI_INFLAMMATORY_RESPONSE_LPS_UP | wilcox_up_in_generic | 31847 | 77 | 439 | 10 | 1.08E−07 | 4.96E−06 |
| HESS_TARGETS_OF_HOXA9_AND_MEIS1_DN | wilcox_up_in_generic | 31847 | 77 | 439 | 10 | 1.08E−07 | 4.96E−06 |
| IVANOVA_HEMATOPOIESIS_EARLY_PROGENITOR | wilcox_up_in_generic | 31847 | 532 | 439 | 25 | 1.31E−07 | 5.77E−06 |
| CELLULAR_PROTEIN_METABOLIC_PROCESS | wilcox_up_in_generic | 31847 | 1117 | 439 | 39 | 1.33E−07 | 5.77E−06 |
| ACEVEDO_LIVER_TUMOR_VS_NORMAL_ADJACENT_{—} | wilcox_up_in_generic | 31847 | 863 | 439 | 33 | 1.69E−07 | 7.21E−06 |
| TISSUE_UP | | | | | | | |
| WANG_IMMORTALIZED_BY_HOXA9_AND_MEIS1_UP | wilcox_up_in_generic | 31847 | 31 | 439 | 7 | 1.78E−07 | 7.36E−06 |
| RYTTCCTG_V$ETS2_B | wilcox_up_in_generic | 31847 | 1085 | 439 | 38 | 1.80E−07 | 7.36E−06 |
| CELLULAR_MACROMOLECULE_METABOLIC_PROCESS | wilcox_up_in_generic | 31847 | 1131 | 439 | 39 | 1.83E−07 | 7.36E−06 |
| KOINUMA_TARGETS_OF_SMAD2_OR_SMAD3 | wilcox_up_in_generic | 31847 | 824 | 439 | 32 | 1.86E−07 | 7.37E−06 |
| KRIGE_RESPONSE_TO_TOSEDOSTAT_24HR_UP | wilcox_up_in_generic | 31847 | 783 | 439 | 31 | 1.91E−07 | 7.45E−06 |
| MODULE_45 | wilcox_up_in_generic | 31847 | 583 | 439 | 26 | 2.03E−07 | 7.76E−06 |
| RASHI_RESPONSE_TO_IONIZING_RADIATION_2 | wilcox_up_in_generic | 31847 | 127 | 439 | 12 | 2.09E−07 | 7.88E−06 |
| RESPONSE_TO_STRESS | wilcox_up_in_generic | 31847 | 508 | 439 | 24 | 2.13E−07 | 7.88E−06 |
| LENAOUR_DENDRITIC_CELL_MATURATION_DN | wilcox_up_in_generic | 31847 | 128 | 439 | 12 | 2.28E−07 | 8.31E−06 |
| MODULE_84 | wilcox_up_in_generic | 31847 | 549 | 439 | 25 | 2.37E−07 | 8.49E−06 |
| GROSS_HYPOXIA_VIA_ELK3_UP | wilcox_up_in_generic | 31847 | 209 | 439 | 15 | 2.48E−07 | 8.76E−06 |
| GTGCCAA, MIR-96 | wilcox_up_in_generic | 31847 | 303 | 439 | 18 | 2.73E−07 | 9.49E−06 |
| MARTENS_TRETINOIN_RESPONSE_DN | wilcox_up_in_generic | 31847 | 841 | 439 | 32 | 2.92E−07 | 1.00E−05 |
| MGGAAGTG_V$GABP_B | wilcox_up_in_generic | 31847 | 757 | 439 | 30 | 2.96E−07 | 1.00E−05 |
| ACEVEDO_LIVER_CANCER_UP | wilcox_up_in_generic | 31847 | 973 | 439 | 35 | 3.01E−07 | 1.00E−05 |
| NEGATIVE_REGULATION_OF_BIOLOGICAL_PROCESS | wilcox_up_in_generic | 31847 | 677 | 439 | 28 | 3.18E−07 | 1.04E−05 |
| MODULE_118 | wilcox_up_in_generic | 31847 | 410 | 439 | 21 | 3.33E−07 | 1.08E−05 |
| GAL_LEUKEMIC_STEM_CELL_DN | wilcox_up_in_generic | 31847 | 244 | 439 | 16 | 3.38E−07 | 1.08E−05 |
| MOOTHA_MITOCHONDRIA | wilcox_up_in_generic | 31847 | 447 | 439 | 22 | 3.44E−07 | 1.08E−05 |
| LINDSTEDT_DENDRITIC_CELL_MATURATION_A | wilcox_up_in_generic | 31847 | 67 | 439 | 9 | 3.49E−07 | 1.08E−05 |
| MEMBRANE | wilcox_up_in_generic | 31847 | 1994 | 439 | 56 | 3.55E−07 | 1.09E−05 |
| TARTE_PLASMA_CELL_VS_PLASMABLAST_DN | wilcox_up_in_generic | 31847 | 309 | 439 | 18 | 3.64E−07 | 1.10E−05 |
| NEGATIVE_REGULATION_OF_CELLULAR_PROCESS | wilcox_up_in_generic | 31847 | 646 | 439 | 27 | 4.22E−07 | 1.25E−05 |
| GRAESSMANN_RESPONSE_TO_MC_AND_DOXORUBICIN_{—} | wilcox_up_in_generic | 31847 | 770 | 439 | 30 | 4.23E−07 | 1.25E−05 |
| DN | | | | | | | |
| GNF2_CD14 | wilcox_up_in_generic | 31847 | 35 | 439 | 7 | 4.35E−07 | 1.26E−05 |
| RPS14_DN.V1_UP | wilcox_up_in_generic | 31847 | 192 | 439 | 14 | 5.14E−07 | 1.47E−05 |
| PUJANA_CHEK2_PCC_NETWORK | wilcox_up_in_generic | 31847 | 779 | 439 | 30 | 5.39E−07 | 1.53E−05 |
| BERENJENO_TRANSFORMED_BY_RHOA_UP | wilcox_up_in_generic | 31847 | 536 | 439 | 24 | 5.57E−07 | 1.56E−05 |
| CELL_DEVELOPMENT | wilcox_up_in_generic | 31847 | 577 | 439 | 25 | 5.92E−07 | 1.64E−05 |
| ONDER_CDH1_TARGETS_2_DN | wilcox_up_in_generic | 31847 | 464 | 439 | 22 | 6.44E−07 | 1.76E−05 |
| REACTOME_ACTIVATED_TLR4_SIGNALLING | wilcox_up_in_generic | 31847 | 93 | 439 | 10 | 6.52E−07 | 1.76E−05 |
| MTOR_UP.N4.V1_UP | wilcox_up_in_generic | 31847 | 196 | 439 | 14 | 6.59E−07 | 1.76E−05 |
| CYTOPLASMIC_PART | wilcox_up_in_generic | 31847 | 1383 | 439 | 43 | 6.89E−07 | 1.82E−05 |
| NEGATIVE_REGULATION_OF_DEVELOPMENTAL_PROCESS | wilcox_up_in_generic | 31847 | 197 | 439 | 14 | 7.00E−07 | 1.83E−05 |
| GNF2_HCK | wilcox_up_in_generic | 31847 | 94 | 439 | 10 | 7.21E−07 | 1.86E−05 |
| COFACTOR_METABOLIC_PROCESS | wilcox_up_in_generic | 31847 | 54 | 439 | 8 | 7.31E−07 | 1.87E−05 |
| SIGNAL_TRANSDUCTION | wilcox_up_in_generic | 31847 | 1634 | 439 | 48 | 7.57E−07 | 1.91E−05 |
| PROGRAMMED_CELL_DEATH | wilcox_up_in_generic | 31847 | 432 | 439 | 21 | 7.76E−07 | 1.94E−05 |

### EXAMPLE 8

Caring potential Efficacy-Related Impacts on Key Immune System Cell Types: GA Induces Tregs more Effectively than PG

We wanted to systematically determine how two different medicines differentially impact immune system cells, using gene expression data. We first utilized an ANOVA-based pattern analysis method to identify a list of genes that are significantly downregulated only by PG compared to medium, and not by GA or GA reference standard compared to medium (Table 10, Methods). We then tested this list for immune system cell type enrichment via a novel approach (Methods). The genes downregulated only by PG are enriched in genes specific for a variety of T cells including Tregs (Table 11). From this same approach, genes significantly upregulated by GA and GA reference standard relative to medium, but not by PG relative to medium (Table 10) again yielded T cells, including Tregs, as the most enriched cell types (Table 11). Finally, genes that have significantly higher expression in samples activated by GA than PG as determined by the 4 parametric methods (Table 8) were, again, enriched for T cells, including Tregs (Table 11).

TABLE 10
Output of the ANOVA pattern matching method utilized to
identify genes upregulated or downregulated only in other
glatiramoid or only in GA and reference standard.
UP IN generic ONLY

| | | Reference | | | Reference | Reference | |
|---|---|---|---|---|---|---|---|
| | | Standard | Medium vs. | Medium vs | Standard vs | Standard vs | GA vs. |
| Gene | ID | pval | GA pval | generic pval | GA pval | generic pval | generic pval |
| 2610027L16RIK | ILMN_2750842 | 0.23316414 | 0.05994357 | 2.2727E−07 | 0.68404036 | 7.9386E−05 | 5.4545E−06 |
| F830002E14RIK | ILMN_1215076 | 0.61979947 | 0.97557017 | 0.00616121 | 0.54021216 | 0.00195298 | 0.00088603 |
| TPSAB1 | ILMN_2705860 | 0.83999437 | 0.11090557 | 1.247E−05 | 0.0501649 | 1.8089E−06 | 5.9979E−05 |
| G6PC3 | ILMN_1218602 | 0.33285147 | 0.07830635 | 0.00023432 | 0.47064847 | 0.00038093 | 0.00034532 |
| MGAT4B | ILMN_2609073 | 0.16909238 | 0.10329483 | 4.5409E−07 | 0.70114033 | 1.5084E−05 | 0.00013637 |
| GM962 | ILMN_2950814 | 0.46743111 | 0.08393291 | 0.00015128 | 0.28108427 | 0.00033705 | 0.00231909 |
| SCMH1 | ILMN_1230766 | 0.85825989 | 0.33943734 | 6.5209E−07 | 0.42348626 | 5.5247E−07 | 1.8627E−07 |
| SLC25A39 | ILMN_2855261 | 0.18484613 | 0.15956452 | 0.00011055 | 0.93089164 | 0.00106198 | 0.00039608 |
| OLFM1 | ILMN_1240615 | 0.36817081 | 0.06923447 | 2.834E−05 | 0.38728168 | 5.0027E−05 | 2.8991E−05 |
| SNCA | ILMN_3161601 | 0.93290826 | 0.98379277 | 0.00327405 | 0.93259129 | 0.00088412 | 7.9207E−05 |
| SNCA | ILMN_3136638 | 0.78545931 | 0.25685494 | 0.00321945 | 0.3721799 | 0.00200127 | 0.00102328 |
| 2310003H01RIK | ILMN_1238640 | 0.18266471 | 0.54629336 | 0.00036557 | 0.29689179 | 0.00239131 | 4.4523E−05 |
| MARCKS | ILMN_1256142 | 0.44405514 | 0.13135479 | 0.00065458 | 0.39346098 | 0.00025084 | 0.00026102 |
| LMAN2L | ILMN_1220769 | 0.37163633 | 0.74201425 | 2.6972E−07 | 0.50497261 | 8.1904E−06 | 8.7497E−08 |
| PDHB | ILMN_3116570 | 0.07045575 | 0.05490528 | 0.00253271 | 0.85039392 | 0.0383537 | 0.01276358 |
| 1300001I01RIK | ILMN_1217040 | 0.15361424 | 0.44508475 | 0.00155067 | 0.43305551 | 0.00992017 | 0.00349757 |
| MARCH2 | ILMN_2618329 | 0.32339884 | 0.05632394 | 0.00054815 | 0.34368315 | 0.00091052 | 0.00248307 |
| REPIN1 | ILMN_2605767 | 0.2862285 | 0.1495528 | 0.00017256 | 0.80716971 | 8.9783E−05 | 5.6491E−05 |
| ALG12 | ILMN_2680686 | 0.21188229 | 0.74975989 | 0.00035696 | 0.17840745 | 0.0038951 | 1.3377E−05 |
| SMARCAL1 | ILMN_2843698 | 0.38176629 | 0.68628203 | 1.6145E−05 | 0.56081924 | 0.00043144 | 2.6809E−05 |
| TRIP10 | ILMN_1213117 | 0.66225544 | 0.06647558 | 0.00013871 | 0.18009813 | 0.00057389 | 0.0012161 |
| PHLPP | ILMN_1237430 | 0.16858802 | 0.54828321 | 0.0007711 | 0.30022427 | 0.00511473 | 0.00035426 |
| WDR6 | ILMN_3162081 | 0.52733239 | 0.29027057 | 0.00031616 | 0.05593257 | 1.6407E−05 | 0.00088629 |
| TRIM28 | ILMN_1227225 | 0.27910558 | 0.10690452 | 8.3413E−05 | 0.56274839 | 0.00027174 | 0.00119331 |
| 1110039B18RIK | ILMN_2790512 | 0.91867384 | 0.71094096 | 0.00011885 | 0.77164086 | 2.3871E−05 | 1.0231E−05 |
| CHMP2A | ILMN_2694998 | 0.09401655 | 0.06695391 | 0.0003865 | 0.83770055 | 0.00700343 | 0.02325497 |
| PIGT | ILMN_2868131 | 0.76706348 | 0.5978573 | 0.00011945 | 0.38694881 | 6.7363E−05 | 0.00031205 |
| LOC100044636 | ILMN_2738554 | 0.07000295 | 0.11971002 | 4.2748E−05 | 0.70497083 | 0.00053411 | 0.00036713 |
| PLEKHO2 | ILMN_2749448 | 0.89586424 | 0.27980336 | 0.00031791 | 0.24578269 | 9.7685E−06 | 0.00017802 |
| EHBP1L1 | ILMN_1227131 | 0.95931483 | 0.11982509 | 0.00158887 | 0.09032625 | 0.00043714 | 0.00990715 |
| ACSL1 | ILMN_2622671 | 0.36515697 | 0.94675499 | 0.00032721 | 0.18512077 | 0.00042494 | 8.0095E−07 |
| 2310016E02RIK | ILMN_1214401 | 0.22980904 | 0.05753222 | 0.00173874 | 0.36518999 | 0.00017037 | 0.0087173 |
| ZFP42 | ILMN_2961604 | 0.23976525 | 0.2201366 | 0.00090477 | 0.85271007 | 0.01568753 | 0.00298353 |
| LOC100044576 | ILMN_2625342 | 0.10741552 | 0.11388429 | 0.0001033 | 0.95747285 | 0.00025062 | 0.00046078 |
| CLECSF12 | ILMN_2663258 | 0.4492669 | 0.13165479 | 0.00075274 | 0.51444064 | 0.00247947 | 0.00173474 |
| RFFL | ILMN_1226372 | 0.88745005 | 0.44898582 | 9.4065E−05 | 0.54637034 | 0.00017954 | 5.4959E−05 |
| MEIS1 | ILMN_1218266 | 0.13926526 | 0.13965044 | 1.4853E−05 | 0.91158609 | 0.00036982 | 0.00106506 |
| EI24 | ILMN_2953677 | 0.20321493 | 0.08234041 | 2.761E−05 | 0.74871296 | 4.6862E−05 | 1.1593E−05 |
| DCP1A | ILMN_2783429 | 0.05690098 | 0.09598543 | 9.7175E−05 | 0.60716706 | 0.00774639 | 0.00098125 |
| 9330147J08RIK | ILMN_1250402 | 0.12066696 | 0.07131906 | 8.2607E−05 | 0.95554097 | 0.00539206 | 0.0011467 |
| NFS1 | ILMN_2648867 | 0.07172253 | 0.05975583 | 0.00027838 | 0.99739104 | 0.01024559 | 0.00973757 |
| YWHAG | ILMN_2587515 | 0.09216541 | 0.08780164 | 0.00018528 | 0.78219017 | 0.02873252 | 0.00578034 |
| FMNL3 | ILMN_2834837 | 0.61470373 | 0.20269367 | 0.00450269 | 0.4743815 | 0.00701983 | 0.00629179 |
| ARF3 | ILMN_1252195 | 0.21207941 | 0.07639583 | 0.00109093 | 0.68850593 | 0.00529829 | 0.00724286 |
| CSF2RA | ILMN_2866276 | 0.96707668 | 0.45619178 | 0.00685185 | 0.3656228 | 0.0020059 | 7.7473E−05 |
| DHRSX | ILMN_1221451 | 0.61770831 | 0.91533017 | 0.00390532 | 0.47911378 | 0.00139201 | 0.0008206 |
| LOC100046035 | ILMN_1242144 | 0.16767262 | 0.05152435 | 0.00102156 | 0.66720065 | 0.02109269 | 0.03091744 |
| 2610019A05RIK | ILMN_2731592 | 0.79476553 | 0.29710653 | 0.0087397 | 0.10987787 | 0.00043363 | 0.01092029 |
| SCHIP1 | ILMN_2937320 | 0.4365044 | 0.59674544 | 0.00264984 | 0.74203514 | 2.2135E−05 | 3.4834E−05 |
| 4632417N05RIK | ILMN_2886896 | 0.33580362 | 0.17497117 | 0.00032764 | 0.70627984 | 0.00086208 | 0.00160253 |
| DORZ1 | ILMN_2773286 | 0.59962186 | 0.34416286 | 2.367E−05 | 0.6534002 | 4.6104E−05 | 0.0001166 |
| CPNE2 | ILMN_2870487 | 0.38065055 | 0.50555105 | 0.00050263 | 0.77488642 | 0.00133283 | 0.00033839 |
| GPD1L | ILMN_2991214 | 0.19977218 | 0.17841498 | 0.00423579 | 0.98352976 | 0.01760926 | 0.01036304 |
| BC018242 | ILMN_2757716 | 0.10342749 | 0.19440643 | 0.00100313 | 0.44546748 | 0.0133763 | 0.00063388 |
| C230090D14 | ILMN_2760468 | 0.13916106 | 0.13594729 | 0.00037764 | 0.78523332 | 0.00774524 | 0.00089867 |
| TSPAN31 | ILMN_2964076 | 0.61807439 | 0.54371572 | 0.01176236 | 0.17458608 | 0.00792563 | 0.00019258 |
| POLB | ILMN_2789425 | 0.21318995 | 0.16314915 | 0.00033153 | 0.84042814 | 0.00381925 | 0.00911601 |
| SIAH1B | ILMN_2903734 | 0.1289666 | 0.57808488 | 0.00287843 | 0.19128214 | 0.03582191 | 0.00122466 |
| LOC383036 | ILMN_2536553 | 0.1482323 | 0.07746285 | 0.000893 | 0.96068675 | 0.04314335 | 0.01082329 |
| LYNX1 | ILMN_1258942 | 0.40779371 | 0.14162429 | 0.00156325 | 0.45570176 | 0.00147629 | 0.01113264 |
| ZFP41 | ILMN_3137980 | 0.26777369 | 0.85153774 | 0.00020126 | 0.22938243 | 0.00120632 | 7.7664E−06 |
| TSPAN33 | ILMN_1219904 | 0.38783856 | 0.07193258 | 0.00113399 | 0.34074726 | 0.00385296 | 0.01315553 |
| MEGF8 | ILMN_2685985 | 0.09269069 | 0.2228606 | 0.00018409 | 0.2853957 | 0.01005303 | 2.9717E−05 |
| GPAA1 | ILMN_1255423 | 0.88572983 | 0.23730731 | 0.01102711 | 0.12664538 | 0.00224226 | 0.03362729 |
| SLC7A8 | ILMN_2598877 | 0.48873112 | 0.31561566 | 0.00252821 | 0.76145739 | 0.00363316 | 0.00378227 |
| RHOF | ILMN_2992047 | 0.78517933 | 0.06471531 | 0.00104676 | 0.06705697 | 0.00017656 | 0.00501895 |
| 2810453I06RIK | ILMN_2510771 | 0.15644308 | 0.10084472 | 0.00323679 | 0.99076785 | 0.01840457 | 0.00616129 |
| PCDHAC1 | ILMN_2980110 | 0.05953082 | 0.23123021 | 0.00031963 | 0.30536078 | 0.01472709 | 0.00058967 |
| SCN1B | ILMN_2834198 | 0.82757251 | 0.93743375 | 0.00109112 | 0.86494367 | 0.00106954 | 0.00044649 |
| CDC42BPB | ILMN_2867696 | 0.2957513 | 0.93597907 | 0.00072093 | 0.26371026 | 8.89E−06 | 0.0001107 |
| BC037704 | ILMN_2470360 | 0.67738795 | 0.74219179 | 0.00010224 | 0.38766078 | 0.00011316 | 1.4832E−06 |
| ADAR | ILMN_3021272 | 0.0580027 | 0.05129306 | 0.00057444 | 0.96259525 | 0.03342987 | 0.02784855 |
| UACA | ILMN_2492716 | 0.35331517 | 0.12247817 | 0.00031066 | 0.62798297 | 0.00213436 | 0.00120635 |
| HIST2H2AA1 | ILMN_1228849 | 0.08845112 | 0.10053728 | 0.00047862 | 0.93209418 | 0.01532418 | 0.0150984 |
| PRDX2 | ILMN_2773554 | 0.13852822 | 0.13387457 | 0.00025021 | 0.89353019 | 0.00624836 | 0.00193576 |
| TFRC | ILMN_2619848 | 0.08516202 | 0.09167884 | 0.00229294 | 0.69987579 | 0.01603902 | 0.00599974 |
| MTAP1S | ILMN_2657432 | 0.6904967 | 0.20031685 | 0.00065825 | 0.05904387 | 8.8765E−05 | 0.00206458 |
| D11WSU47E | ILMN_2700833 | 0.225963 | 0.14304693 | 0.000121 | 0.86283361 | 0.00363177 | 0.00317771 |
| ADPRHL2 | ILMN_2639063 | 0.68405179 | 0.3473478 | 0.00539446 | 0.11863376 | 0.00089913 | 0.00540034 |
| SYMPK | ILMN_1245437 | 0.71977433 | 0.59368169 | 0.00101082 | 0.891984 | 0.00600606 | 0.002917 |
| GYPA | ILMN_2696696 | 0.73890142 | 0.36327688 | 0.02519012 | 0.4987125 | 0.00319309 | 0.00035484 |
| KLHDC3 | ILMN_2851023 | 0.78302203 | 0.24372306 | 0.00376383 | 0.26988503 | 0.00042878 | 0.00806745 |
| 3110070M22RIK | ILMN_2696066 | 0.10737962 | 0.20502087 | 0.00145115 | 0.48660361 | 0.0398205 | 0.00352369 |
| TRUB1 | ILMN_1240735 | 0.29536566 | 0.07343895 | 9.2393E−05 | 0.56103171 | 0.00086925 | 0.00063531 |
| AKT2 | ILMN_2655895 | 0.84429708 | 0.25772291 | 4.5462E−05 | 0.34370849 | 4.3497E−05 | 1.5323E−07 |
| RSC1A1 | ILMN_1230668 | 0.68401145 | 0.05392083 | 0.00042967 | 0.0858665 | 0.00013655 | 0.00201638 |
| FRY | ILMN_2623288 | 0.41843328 | 0.17162959 | 0.00085191 | 0.58088139 | 0.0035019 | 0.00563097 |
| TJAP1 | ILMN_2934448 | 0.46480666 | 0.48001853 | 0.0005054 | 0.10395886 | 0.0031371 | 7.3799E−06 |
| CNGA4 | ILMN_2875896 | 0.41280262 | 0.838024 | 0.00421111 | 0.4856524 | 0.02626848 | 0.00310152 |
| SGSM3 | ILMN_2987294 | 0.2852907 | 0.17993849 | 0.00055295 | 0.8524482 | 0.00238157 | 0.00201576 |
| 9430078K10RIK | ILMN_2500041 | 0.5504554 | 0.11642124 | 0.00175458 | 0.30830794 | 0.0041732 | 0.01656268 |
| SLC26A6 | ILMN_2668261 | 0.35123184 | 0.37483965 | 0.00205543 | 0.93052826 | 0.01382185 | 0.01126309 |
| LYSMD4 | ILMN_2701435 | 0.32638926 | 0.7070625 | 0.0012552 | 0.36786548 | 0.00282183 | 6.4684E−05 |
| PCBD2 | ILMN_2876775 | 0.83857904 | 0.06857342 | 0.00093677 | 0.05659279 | 7.0666E−05 | 0.0052888 |
| UAP1 | ILMN_2502542 | 0.21657246 | 0.98541869 | 0.00090753 | 0.13404544 | 0.03188188 | 0.00024728 |
| RNF40 | ILMN_1242623 | 0.211788 | 0.09917397 | 0.00010844 | 0.65795314 | 0.00010746 | 0.00071064 |
| LOC277049 | ILMN_1240399 | 0.29345712 | 0.10601588 | 0.00032209 | 0.57559205 | 0.00299319 | 0.00566302 |
| LOC100047603 | ILMN_2697057 | 0.5317771 | 0.49723284 | 0.00279969 | 0.90335285 | 0.00408611 | 0.00028596 |
| BCL2L12 | ILMN_2935909 | 0.4501663 | 0.15075832 | 0.00083123 | 0.54081523 | 0.00491744 | 0.00699663 |
| HMGCL | ILMN_1256234 | 0.29797373 | 0.38742722 | 0.00288991 | 0.87354805 | 0.01353055 | 0.02023386 |
| TRPV4 | ILMN_2466284 | 0.25145055 | 0.33803647 | 0.00031314 | 0.68273446 | 0.01883637 | 0.00211203 |
| 9030624J02RIK | ILMN_2872639 | 0.78200401 | 0.41703987 | 0.00011443 | 0.60307495 | 0.00035665 | 0.00031891 |
| 11110020P15RIK | ILMN_3142224 | 0.12120649 | 0.0502281 | 3.9974E−06 | 0.98580392 | 0.00101571 | 5.2276E−05 |
| SNCA | ILMN_3059393 | 0.26266042 | 0.20886172 | 4.5363E−05 | 0.89343173 | 0.00301563 | 0.00397248 |
| 9130011J15RIK | ILMN_2593484 | 0.23649453 | 0.43542115 | 0.00079019 | 0.48432507 | 0.00494982 | 0.00016332 |
| ATP9B | ILMN_1233770 | 0.82506746 | 0.1176255 | 0.00078987 | 0.14652888 | 0.00044743 | 0.00317972 |
| KCNC3 | ILMN_2681742 | 0.372855 | 0.7150701 | 0.00180644 | 0.48590107 | 0.00820357 | 0.00055463 |
| LOC100045460 | ILMN_1216433 | 0.51010123 | 0.3577678 | 0.00102881 | 0.0673625 | 3.8938E−05 | 0.00093652 |
| MAGEE2 | ILMN_2477690 | 0.17108453 | 0.05934004 | 0.00118353 | 0.60110983 | 0.00727604 | 0.01024961 |
| LOC100047194 | ILMN_1217481 | 0.1593328 | 0.09817286 | 1.4053E−05 | 0.93984431 | 0.01188776 | 0.0069427 |
| MS4A7 | ILMN_3091003 | 0.57297521 | 0.48201756 | 0.01377112 | 0.89723565 | 0.02317278 | 0.02103231 |
| EFCAB6 | ILMN_2928498 | 0.76323421 | 0.32217211 | 0.00439564 | 0.48635484 | 0.00343022 | 0.00251696 |
| CEBPE | ILMN_3029849 | 0.80783743 | 0.17328494 | 0.00307707 | 0.23187011 | 0.002828 | 0.01622655 |
| LOC100046781 | ILMN_249142 | 0.6583194 | 0.83343519 | 0.00149683 | 0.46895205 | 0.00385634 | 0.00039912 |
| C920004C08RIK | ILMN_1222071 | 0.38502878 | 0.07005445 | 0.0038178 | 0.38809925 | 0.01253032 | 0.02259007 |
| TNXB | ILMN_2444411 | 0.14961479 | 0.10546876 | 0.00050175 | 0.88054682 | 0.00213799 | 0.00191327 |
| ADAL | ILMN_2948663 | 0.23696542 | 0.17163928 | 0.0033817 | 0.97176484 | 0.0463896 | 0.01802189 |
| INTS7 | ILMN_2726174 | 0.27324071 | 0.586879 | 0.00587044 | 0.41797982 | 0.0040954 | 0.00107415 |
| FOXP4 | ILMN_2650317 | 0.74594364 | 0.56270629 | 0.01705487 | 0.75513764 | 0.00235308 | 0.00528704 |
| Ageneric5L | ILMN_1217612 | 0.83299323 | 0.9146392 | 0.00048051 | 0.69535158 | 3.7093E−05 | 1.4826E−05 |
| CD44 | ILMN_2474229 | 0.26929639 | 0.14781976 | 0.00554927 | 0.92553119 | 0.04471501 | 0.01739387 |
| ZMAT3 | ILMN_2418426 | 0.25658442 | 0.86316426 | 0.00223223 | 0.09948234 | 0.02695902 | 8.304E−05 |
| PRPF3 | ILMN_2941431 | 0.48039198 | 0.0666872 | 0.00191681 | 0.1846098 | 0.0003537 | 0.00722913 |
| RPUSD3 | ILMN_1214740 | 0.2304027 | 0.23279576 | 0.00255484 | 0.83257185 | 0.01690345 | 0.00491704 |
| ZBTB6 | ILMN_2525819 | 0.13087182 | 0.12258692 | 0.00141696 | 0.69934155 | 0.01954111 | 0.00144419 |
| MAGEA9 | ILMN_1252512 | 0.83094731 | 0.08459744 | 0.00068872 | 0.11884211 | 0.00138663 | 0.01801554 |
| PTPRF | ILMN_1219189 | 0.21904246 | 0.08672444 | 0.00056349 | 0.5640646 | 0.00429561 | 0.0157663 |
| NSF | ILMN_2760629 | 0.19318587 | 0.36073604 | 0.00085745 | 0.55958345 | 0.00518866 | 0.00095739 |
| SARDH | ILMN_2903455 | 0.79605022 | 0.77858591 | 0.01016045 | 0.51361001 | 0.00130347 | 0.00283633 |
| XPO5 | ILMN_1216198 | 0.70505686 | 0.67920197 | 0.02384337 | 0.33063911 | 0.04204905 | 0.00057367 |
| VGLL2 | ILMN_2495753 | 0.34936137 | 0.26151967 | 0.00612462 | 0.84191343 | 0.01019352 | 0.02417299 |
| HSD3B2 | ILMN_1233611 | 0.38575077 | 0.84652261 | 0.00014189 | 0.19970449 | 0.00477567 | 4.8549E−06 |
| CAPZB | ILMN_1230100 | 0.1875435 | 0.06034991 | 0.00217355 | 0.67582845 | 0.02773325 | 0.03238359 |
| PKIB | ILMN_3052870 | 0.70096129 | 0.27928246 | 0.00023369 | 0.45016594 | 0.00041464 | 0.0035889 |
| A230005A19RIK | ILMN_2564767 | 0.63802511 | 0.18917144 | 2.6475E−05 | 0.05538688 | 1.1591E−05 | 8.0443E−05 |
| USP10 | ILMN_1217931 | 0.79465925 | 0.83880456 | 0.00962728 | 0.93399997 | 0.00955342 | 0.00642993 |
| NCDN | ILMN_1231405 | 0.81698433 | 0.416902 | 0.00667762 | 0.20351527 | 0.00022093 | 0.00778456 |
| SYN1 | ILMN_1235133 | 0.66948488 | 0.25789954 | 0.00450181 | 0.07793351 | 0.00082228 | 0.00995117 |
| ARVCF | ILMN_2573293 | 0.44694609 | 0.75666837 | 0.00102822 | 0.62945864 | 0.00334104 | 0.00086306 |
| SMG7 | ILMN_2953017 | 0.42587972 | 0.38068927 | 0.01889443 | 0.9166145 | 0.02184543 | 0.00802694 |
| 5730472N09RIK | ILMN_2765936 | 0.40839264 | 0.60823708 | 0.00194252 | 0.6700575 | 0.01253481 | 0.00179129 |
| ZMIZ1 | ILMN_1224736 | 0.37634424 | 0.05504898 | 0.01366886 | 0.39849773 | 0.00224248 | 9.7983E−06 |
| ZFP162 | ILMN_1251244 | 0.20140467 | 0.74349021 | 0.003566 | 0.22753522 | 1.5161E−05 | 0.00014626 |
| DUS2L | ILMN_1256843 | 0.85616436 | 0.44642801 | 0.0038192 | 0.27855692 | 0.00086407 | 0.00172912 |
| BZRPL1 | ILMN_2635718 | 0.61850682 | 0.57423004 | 0.01900537 | 0.90318112 | 0.04301692 | 0.00446837 |
| ARFGAP2 | ILMN_2853409 | 0.8237825 | 0.85158881 | 0.00199602 | 0.94478983 | 0.00678872 | 0.00130004 |
| 9330183F02RIK | ILMN_2558866 | 0.32614679 | 0.17334775 | 0.00055707 | 0.63326394 | 0.00580031 | 0.01681826 |
| DBF4 | ILMN_2952661 | 0.62403934 | 0.83848649 | 0.01229066 | 0.39859235 | 0.01913089 | 0.00166558 |
| ATG3 | ILMN_2595188 | 0.62133153 | 0.90315959 | 0.00841756 | 0.57722036 | 0.0012036 | 0.00020877 |
| ISCA1 | ILMN_2888842 | 0.35957584 | 0.78095788 | 0.00762094 | 0.43658095 | 0.03725391 | 0.00503629 |
| 4930544L04RIK | ILMN_1256258 | 0.12121171 | 0.14441762 | 0.0006835 | 0.73082687 | 0.03198867 | 0.00668889 |
| 2610209A20RIK | ILMN_2744228 | 0.22225103 | 0.05035008 | 0.00386646 | 0.63077254 | 0.03886407 | 0.03001504 |
| 2810046M22RIK | ILMN_2714278 | 0.79325707 | 0.3413647 | 0.00690207 | 0.43065552 | 0.00213172 | 0.00722839 |
| DHDH | ILMN_1234086 | 0.74724453 | 0.59059753 | 0.00175229 | 0.86971555 | 0.00020788 | 4.2838E−06 |
| ALOX15 | ILMN_2789023 | 0.49450876 | 0.53638936 | 0.00570701 | 0.91093104 | 0.00801067 | 0.00525598 |
| RNF183 | ILMN_2753931 | 0.26921908 | 0.18842993 | 0.00686923 | 0.88653996 | 0.02011683 | 0.02538967 |
| LOC385989 | ILMN_2538010 | 0.18607303 | 0.20021422 | 0.00191936 | 0.75816273 | 0.02628028 | 0.0047078 |
| RBPMS | ILMN_3026557 | 0.96763167 | 0.56224954 | 0.00348847 | 0.54346053 | 0.00072125 | 8.217E−06 |
| MTRR | ILMN_2969314 | 0.13801396 | 0.28082362 | 0.00274793 | 0.44877156 | 0.04540035 | 0.00332952 |
| 5930400G23RIK | ILMN_1252245 | 0.40467526 | 0.46528262 | 0.00347765 | 0.84652383 | 0.03143047 | 0.01143447 |
| CD151 | ILMN_2492403 | 0.92119216 | 0.67348082 | 0.01261715 | 0.55087141 | 0.00528279 | 0.00145378 |
| LOC380636 | ILMN_2533773 | 0.12145288 | 0.08809138 | 0.00055433 | 0.99484243 | 0.02332253 | 0.01160268 |
| ITFG2 | ILMN_1252621 | 0.7616105 | 0.4091743 | 0.00122209 | 0.19592237 | 0.00020626 | 0.00083074 |
| PALM | ILMN_2815626 | 0.27210377 | 0.60175917 | 0.00702535 | 0.05264141 | 9.9305E−05 | 0.00462694 |
| ZFP563 | ILMN_2642938 | 0.193144 | 0.30579008 | 0.00389083 | 0.60583451 | 0.0392958 | 0.00644165 |
| LOC382813 | ILMN_1237743 | 0.32446022 | 0.13282026 | 0.00113931 | 0.66957638 | 6.7587E−05 | 1.9804E−06 |
| BC017612 | ILMN_2631514 | 0.83552119 | 0.85013235 | 0.01809313 | 0.66005927 | 0.00993053 | 0.00823769 |
| CBARA1 | ILMN_2686513 | 0.86139711 | 0.9466614 | 0.01799514 | 0.88573743 | 0.00710622 | 0.0037732 |
| COQ9 | ILMN_2774456 | 0.59075852 | 0.13753291 | 0.00086124 | 0.34900387 | 0.00621783 | 0.02796696 |
| USP36 | ILMN_2491232 | 0.56949259 | 0.38609624 | 0.03027487 | 0.07311196 | 0.04192555 | 0.00017844 |
| 1700063H04RIK | ILMN_1244141 | 0.63515532 | 0.10809724 | 0.00890166 | 0.2270816 | 0.00885167 | 0.03437235 |
| JPH1 | ILMN_1217728 | 0.34365308 | 0.09716164 | 0.00319925 | 0.53577226 | 0.01378973 | 0.02153318 |
| CLEC4A3 | ILMN_3161652 | 0.11282409 | 0.62228735 | 0.04844097 | 0.11660557 | 0.00026547 | 0.00217505 |
| R74862 | ILMN_2745522 | 0.43139805 | 0.38262131 | 0.0063334 | 0.05657433 | 0.00019849 | 0.01574341 |
| LOC332300 | ILMN_2531139 | 0.21628289 | 0.45088627 | 0.00051207 | 0.63201644 | 0.012255 | 0.00428559 |
| SPEN | ILMN_2592059 | 0.17066734 | 0.96706426 | 0.02340849 | 0.06264559 | 0.0001212 | 0.00366195 |
| CHRM2 | ILMN_2971508 | 0.55099734 | 0.40333119 | 0.00143259 | 0.11974918 | 0.00255277 | 5.0378E−05 |
| PSG22 | ILMN_3078396 | 0.15765766 | 0.46614787 | 0.00173384 | 0.41177633 | 0.03160967 | 0.00316651 |
| E330037I15RIK | ILMN_2584027 | 0.13994579 | 0.90103078 | 0.00116894 | 0.07547583 | 0.01568279 | 7.005E−05 |
| SCAVENGER RECEPTOR | ILMN_2449412 | 0.06423164 | 0.51146019 | 0.01084533 | 0.08950745 | 2.5558E−07 | 4.5499E−05 |
| UBE2E2 | ILMN_2792485 | 0.66434328 | 0.95217738 | 0.00650482 | 0.64814483 | 0.00202822 | 0.00104592 |
| PALLD | ILMN_1246642 | 0.16878379 | 0.27707286 | 0.00073811 | 0.68212951 | 0.03311922 | 0.00983465 |
| REXO1 | ILMN_1245568 | 0.55805948 | 0.83302065 | 0.01440805 | 0.33571906 | 0.00192893 | 0.00692206 |
| 9330104G04RIK | ILMN_1255975 | 0.39302814 | 0.05223332 | 0.00019054 | 0.27198445 | 0.0027408 | 0.01737767 |
| PKIA | ILMN_2674122 | 0.67293267 | 0.4629903 | 0.00556462 | 0.1917034 | 0.00108801 | 0.01215542 |
| 1200003C05RIK | ILMN_2943319 | 0.59622266 | 0.14302272 | 0.01192579 | 0.29485229 | 0.00853701 | 0.04235168 |
| C130040D06RIK | ILMN_2746366 | 0.80272538 | 0.22751982 | 0.00284699 | 0.36441676 | 0.00995222 | 0.01576115 |
| MOCO5 | ILMN_2906489 | 0.75542925 | 0.32906964 | 0.00350144 | 0.49760379 | 0.00128916 | 6.2052E−05 |
| PCOLN3 | ILMN_2847502 | 0.66324539 | 0.50606993 | 0.04235283 | 0.15162103 | 0.00125839 | 0.03154406 |
| DACH1 | ILMN_3072197 | 0.33621604 | 0.35503485 | 0.00110316 | 0.91837597 | 0.00900515 | 0.00666164 |
| B930062B16RIK | ILMN_1220808 | 0.32790334 | 0.08497289 | 0.00217034 | 0.4370566 | 0.00723409 | 0.02820519 |
| UGP2 | ILMN_1244631 | 0.79817532 | 0.5705628 | 0.00365856 | 0.76237104 | 0.00489094 | 0.00380639 |
| RALGPS2 | ILMN_2884869 | 0.83773462 | 0.64566493 | 0.00759159 | 0.77764494 | 0.00171812 | 0.00212375 |
| MFSD7A | ILMN_2766105 | 0.60693902 | 0.58513362 | 0.03367774 | 0.9448507 | 0.03040298 | 0.01214659 |
| KREMEN2 | ILMN_2951946 | 0.97572697 | 0.86706274 | 0.0142853 | 0.82630834 | 0.01010007 | 0.00233333 |
| LOC223628 | ILMN_1216043 | 0.2465434 | 0.88909294 | 0.00156273 | 0.23866081 | 0.00952059 | 0.00085508 |
| RASAL2 | ILMN_1233634 | 0.13878571 | 0.21684543 | 0.00076632 | 0.66394521 | 0.01748223 | 0.00333495 |
| OLFR1155 | ILMN_1226210 | 0.60008585 | 0.26727264 | 0.00046584 | 0.56713115 | 0.00163827 | 0.00230214 |
| 1700021P22RIK | ILMN_1237313 | 0.72320123 | 0.43628571 | 0.00836603 | 0.6174392 | 0.00399991 | 0.01898456 |
| 9430088N01RIK | ILMN_1251471 | 0.18310252 | 0.16561225 | 0.0028294 | 0.96478484 | 0.00932456 | 0.01549275 |
| MKNK1 | ILMN_2547867 | 0.33749491 | 0.3062801 | 0.01310828 | 0.88788853 | 0.03685846 | 0.01320064 |
| FAHD1 | ILMN_1218891 | 0.60928218 | 0.65835483 | 0.00558641 | 0.95628689 | 0.00626434 | 0.00627596 |
| 4930432K21RIK | ILMN_2672745 | 0.64921947 | 0.86232486 | 0.01529869 | 0.69522914 | 0.02043091 | 0.00321814 |
| KRTCAP2 | ILMN_2605694 | 0.48175968 | 0.26514408 | 0.00107793 | 0.72772374 | 0.0009224 | 0.0005065 |
| LY96 | ILMN_2743003 | 0.60240232 | 0.37156798 | 0.02495881 | 0.1045283 | 0.00420396 | 0.038316 |
| TAF3 | ILMN_3160989 | 0.42511261 | 0.87443846 | 0.00506607 | 0.27553271 | 0.01219759 | 0.00149854 |
| 1200011M11RIK | ILMN_3105117 | 0.80288013 | 0.67937454 | 0.01906433 | 0.46899481 | 0.03049915 | 0.00367463 |
| 4930430K04RIK | ILMN_1250290 | 0.37545277 | 0.72073779 | 0.00762228 | 0.38313585 | 0.01192779 | 0.00035218 |
| FGR | ILMN_1244821 | 0.95106768 | 0.41582138 | 0.00561853 | 0.44221591 | 0.01073639 | 0.02742569 |
| 5830457O10RIK | ILMN_2697766 | 0.361932 | 0.53781105 | 0.02816252 | 0.69991901 | 6.469E−05 | 0.00086576 |
| FBXL21 | ILMN_2518214 | 0.1497671 | 0.07565383 | 0.00162512 | 0.86714862 | 0.02552817 | 0.02051226 |
| BC031853 | ILMN_2764549 | 0.05063828 | 0.64726274 | 0.00431543 | 0.09295443 | 4.8105E−06 | 0.00071839 |
| LOC546088 | ILMN_2533350 | 0.72822666 | 0.44487114 | 0.00928651 | 0.688774 | 0.01706508 | 0.01512498 |
| control_ILMN_1378661 | ILMN_1378661 | 0.58288344 | 0.56218964 | 0.00115548 | 0.98721187 | 0.00064345 | 0.00022549 |
| HTATIP2 | ILMN_2603834 | 0.47453234 | 0.27654833 | 0.00141767 | 0.76406644 | 0.00210299 | 0.00161854 |
| CENTD3 | ILMN_1243107 | 0.30658868 | 0.18469695 | 0.0070268 | 0.90534006 | 0.00051431 | 2.391E−05 |
| DNAHC17 | ILMN_1215848 | 0.78560788 | 0.94735885 | 0.00042695 | 0.7754273 | 0.00104722 | 2.0647E−05 |
| 5330403O16RIK | ILMN_1241724 | 0.23088372 | 0.14548399 | 0.00380189 | 0.90437295 | 0.04190745 | 0.02609541 |
| CCL2 | ILMN_1245710 | 0.68115941 | 0.35160796 | 0.0180609 | 0.15481728 | 0.00601552 | 0.04458017 |
| C230062L16RIK | ILMN_2582211 | 0.79458172 | 0.83053644 | 0.00735606 | 0.91122432 | 0.01026268 | 0.00140343 |
| 1700029G01RIK | ILMN_1250435 | 0.76182257 | 0.97237019 | 0.01032863 | 0.68682607 | 0.01011578 | 0.0025475 |
| 9330158H04RIK | ILMN_2660943 | 0.64512593 | 0.5227084 | 0.00152554 | 0.95365122 | 0.00750219 | 0.00088507 |
| ENAH | ILMN_2636853 | 0.06868458 | 0.33056646 | 0.00163374 | 0.34847938 | 0.04629448 | 0.00818286 |
| SLC13A5 | ILMN_1229923 | 0.94509976 | 0.60925235 | 0.01845312 | 0.49303532 | 0.004652 | 0.00701722 |
| 6720407G21RIK | ILMN_1245282 | 0.8866404 | 0.40188413 | 0.00068075 | 0.46800238 | 0.00022872 | 1.8039E−06 |
| BTRC | ILMN_3100042 | 0.34472936 | 0.67810519 | 0.01282088 | 0.52994813 | 0.00029857 | 0.00143989 |
| COTL1 | ILMN_2915232 | 0.79492031 | 0.55583484 | 0.00718325 | 0.77390032 | 0.01512099 | 0.00754377 |
| NEK4 | ILMN_2634859 | 0.91526202 | 0.19311108 | 0.00578328 | 0.12445123 | 0.00292968 | 0.03295181 |
| 6430709H04RIK | ILMN_2609850 | 0.89780111 | 0.41535609 | 0.00121402 | 0.50801567 | 0.0039779 | 0.00293725 |
| TCEAL5 | ILMN_2630772 | 0.97219594 | 0.442065 | 0.00556045 | 0.36220998 | 0.00150024 | 0.01076635 |
| NFE2L1 | ILMN_1251729 | 0.35151102 | 0.88469349 | 0.00095891 | 0.3619295 | 0.00731206 | 0.00040477 |
| THOC1 | ILMN_2850527 | 0.83855663 | 0.48870524 | 0.01990776 | 0.61754068 | 0.01368403 | 0.01084815 |
| CTNNA3 | ILMN_2834525 | 0.35364536 | 0.37546314 | 0.01198092 | 0.8661483 | 0.0305254 | 0.01046438 |
| 9430003J03RIK | ILMN_1258022 | 0.65571138 | 0.28190997 | 0.00195284 | 0.48961492 | 0.00186634 | 0.00713957 |
| NAPRT1 | ILMN_2799590 | 0.96014189 | 0.33076081 | 0.00799053 | 0.26651831 | 0.00616399 | 0.00032339 |
| 5YNGR1 | ILMN_3101732 | 0.9391257 | 0.35082575 | 0.00984298 | 0.30041757 | 0.01328617 | 0.03036858 |
| LOC380723 | ILMN_1243160 | 0.37740932 | 0.67618749 | 0.00143845 | 0.516955 | 0.00779308 | 0.00029654 |
| D130017N08RIK | ILMN_1247385 | 0.16667108 | 0.48307406 | 0.01019145 | 0.42178833 | 0.04400345 | 0.01404355 |
| LOC677528 | ILMN_2656362 | 0.47982841 | 0.94578199 | 0.00715636 | 0.41450757 | 0.00026806 | 0.00103661 |
| BCL2L2 | ILMN_2551387 | 0.22818583 | 0.21524422 | 0.00473619 | 0.97372329 | 0.01300616 | 0.01597522 |
| ZEB2 | ILMN_1227793 | 0.65765685 | 0.57181172 | 0.00049367 | 0.92187971 | 0.00195583 | 0.00109947 |
| UBE2A | ILMN_2476804 | 0.81778966 | 0.95000461 | 0.01743581 | 0.74057972 | 0.01722272 | 0.01181808 |
| BC040758 | ILMN_3034303 | 0.28931872 | 0.37692576 | 0.00522104 | 0.79818174 | 0.03721202 | 0.01925994 |
| FOXE1 | ILMN_3163279 | 0.84308178 | 0.94893077 | 0.01799077 | 0.74930041 | 0.0116943 | 0.00509161 |
| BCKDK | ILMN_1249301 | 0.21758878 | 0.92229557 | 0.01550038 | 0.11873778 | 8.6233E−05 | 0.00580959 |
| TRIM15 | ILMN_1246816 | 0.68974946 | 0.12959121 | 0.00201966 | 0.23014116 | 0.00229519 | 0.01771175 |
| HOXC10 | ILMN_2836885 | 0.89837669 | 0.70710511 | 0.03140007 | 0.59043209 | 0.04308878 | 0.00589081 |
| NFKB1 | ILMN_2592476 | 0.76898705 | 0.95041769 | 0.00295371 | 0.63790578 | 0.00790483 | 6.3164E−05 |
| OLFR228 | ILMN_2818216 | 0.27737328 | 0.61973791 | 0.00249111 | 0.06119513 | 6.1572E−05 | 0.00291443 |
| CLCN1 | ILMN_2802672 | 0.49446288 | 0.9550135 | 0.00484028 | 0.46793233 | 0.04129309 | 0.00345529 |
| 6330579M01RIK | ILMN_1256365 | 0.37596201 | 0.83897985 | 0.00183326 | 0.22227265 | 0.00759449 | 0.00017292 |
| SFRS2IP | ILMN_2713448 | 0.73748419 | 0.41559133 | 0.01633789 | 0.15302115 | 0.00738233 | 7.5446E−05 |
| 4930543G11RIK | ILMN_1233640 | 0.82294632 | 0.6641014 | 0.00221225 | 0.43702216 | 0.00151109 | 1.1977E−05 |
| 2900090M10RIK | ILMN_1260170 | 0.28438878 | 0.10514918 | 0.00066848 | 0.70176192 | 0.02144135 | 0.02098325 |
| LOC100048331 | ILMN_1242041 | 0.58901281 | 0.84782172 | 0.02603217 | 0.37387194 | 0.02981401 | 0.00389063 |
| TFIP11 | ILMN_2661666 | 0.90213352 | 0.16019485 | 0.00593209 | 0.07447876 | 0.00064179 | 0.04218803 |
| TTC4 | ILMN_1220820 | 0.50583431 | 0.99873725 | 0.01579862 | 0.45381957 | 0.0009629 | 0.00593889 |
| CAMK1 | ILMN_2944939 | 0.10460814 | 0.15798962 | 0.02459022 | 0.52801525 | 5.1199E−05 | 1.8481E−05 |
| 2600005J22RIK | ILMN_1246515 | 0.84043545 | 0.16470974 | 0.00930877 | 0.15062039 | 0.00127562 | 0.04579512 |
| PSCD2 | ILMN_1251874 | 0.34994684 | 0.4129054 | 0.00566457 | 0.77170559 | 0.00091644 | 0.00017891 |
| control_ILMN_1378911 | ILMN_1378911 | 0.32667688 | 0.42471309 | 0.00428809 | 0.8607028 | 0.01345908 | 0.00757724 |
| F830116E18RIK | ILMN_2905525 | 0.48967608 | 0.31908374 | 0.01678449 | 0.90510364 | 0.00993299 | 0.00076042 |
| ATPBD3 | ILMN_2783557 | 0.75585531 | 0.88699079 | 0.01683452 | 0.81877079 | 0.02746112 | 0.00722026 |
| SLC22A21 | ILMN_1234533 | 0.17026988 | 0.82540457 | 0.00925147 | 0.09451465 | 0.03706086 | 0.00051334 |
| FBXL15 | ILMN_2792047 | 0.42348825 | 0.52124517 | 0.01450012 | 0.85818541 | 0.00084336 | 0.0015336 |
| 4930481A15RIK | ILMN_1248737 | 0.61289473 | 0.6812972 | 0.00651844 | 0.88723651 | 0.00423683 | 0.00330588 |
| E030016N13RIK | ILMN_1235317 | 0.73525443 | 0.36765944 | 0.01795379 | 0.58316034 | 0.01007815 | 0.00114404 |
| PDGFB | ILMN_2618714 | 0.95021682 | 0.63736944 | 0.01466175 | 0.6615403 | 0.00915126 | 0.0067522 |
| 4933437N03RIK | ILMN_2774580 | 0.59904062 | 0.40840008 | 0.03995368 | 0.72366676 | 0.02244034 | 0.04125272 |
| 2310046A06RIK | ILMN_2700344 | 0.09693427 | 0.3378204 | 0.00361157 | 0.40525941 | 9.8372E−06 | 2.6431E−05 |
| MAK10 | ILMN_2828599 | 0.57524627 | 0.99831228 | 0.00462742 | 0.4325795 | 3.7751E−05 | 8.2705E−05 |
| LOC277236 | ILMN_2530354 | 0.74572568 | 0.6674914 | 0.00507727 | 0.97771727 | 0.01683661 | 0.00305436 |
| TMEM43 | ILMN_2700505 | 0.08359427 | 0.06267175 | 0.00363649 | 0.72501753 | 0.04936703 | 0.00890644 |
| CAMK1 | ILMN_2671721 | 0.44365063 | 0.55451755 | 0.01324093 | 0.75702043 | 0.0275119 | 0.01087233 |
| SPIN2 | ILMN_2934522 | 0.63405541 | 0.99500552 | 0.02510773 | 0.54232848 | 0.02921252 | 0.00445866 |
| V1RC23 | ILMN_3004978 | 0.87375928 | 0.74179796 | 0.01065243 | 0.86417002 | 0.01983656 | 0.018027 |
| ZFP239 | ILMN_3127381 | 0.58169905 | 0.66161311 | 0.0039776 | 0.24080108 | 0.00012244 | 0.0024932 |
| COQ6 | ILMN_2766720 | 0.06092969 | 0.17676374 | 0.00051581 | 0.56433332 | 0.02938357 | 0.01500906 |
| CLCNKB | ILMN_2757283 | 0.52418848 | 0.77111416 | 0.00944684 | 0.32960104 | 0.0020966 | 0.02196419 |
| 2310051M13RIK | ILMN_1237270 | 0.96654677 | 0.77123524 | 0.01036298 | 0.71444946 | 0.00836162 | 0.00059521 |
| RHOT1 | ILMN_1242500 | 0.08801159 | 0.11586897 | 0.00211474 | 0.74329166 | 0.03708177 | 0.01498654 |
| GPR106, LGR8, GPCR | ILMN_1232852 | 0.65598321 | 0.19431475 | 0.00488593 | 0.37987832 | 0.00751158 | 0.01854805 |
| OLFR907 | ILMN_2598615 | 0.80860498 | 0.15586787 | 0.0060448 | 0.07493401 | 0.00331944 | 0.03561223 |
| TRABD | ILMN_2978533 | 0.99327612 | 0.62026479 | 0.02986947 | 0.57568866 | 0.01868526 | 0.00703553 |
| BLCAP | ILMN_2705802 | 0.20007345 | 0.91664338 | 0.00728425 | 0.06692845 | 4.6061E−05 | 0.00060719 |
| GM628 | ILMN_3160644 | 0.5486986 | 0.37399695 | 0.0198613 | 0.8226972 | 0.04707652 | 0.04340188 |
| DNM3 | ILMN_2482131 | 0.25766404 | 0.2129101 | 0.0029941 | 0.95020206 | 0.02525937 | 0.02711162 |
| MCPT6 | ILMN_3161036 | 0.30757692 | 0.33380968 | 0.01639779 | 0.91289935 | 0.00191872 | 0.00123825 |
| MAPKAPK2 | ILMN_2614212 | 0.34056978 | 0.97898611 | 0.01443653 | 0.22707081 | 0.00026851 | 0.00363695 |
| FAM123A | ILMN_2456075 | 0.40994995 | 0.98420717 | 0.00554166 | 0.33936861 | 0.01613788 | 0.00116246 |
| SAMD10 | ILMN_2656359 | 0.12270684 | 0.75105295 | 0.03336925 | 0.07397494 | 0.00084455 | 0.00232642 |
| ZFP748 | ILMN_3083372 | 0.95058107 | 0.72301296 | 0.01522827 | 0.75282482 | 0.01128098 | 0.0166564 |
| MLANA | ILMN_2624504 | 0.83660637 | 0.97173682 | 0.03425913 | 0.77240455 | 0.00945458 | 0.01168146 |
| ITGA2B | ILMN_2911123 | 0.55235292 | 0.8240584 | 0.02808366 | 0.67671548 | 0.00633087 | 0.01162745 |
| CYGB | ILMN_2801891 | 0.79708364 | 0.57972409 | 0.02849663 | 0.37389718 | 0.01698838 | 0.04977838 |
| ATP13A1 | ILMN_1250382 | 0.50488802 | 0.53042647 | 0.01377508 | 0.95673298 | 0.02788979 | 0.02618921 |
| TRIM2 | ILMN_1234848 | 0.86351136 | 0.72585714 | 0.04803533 | 0.56904864 | 0.03649278 | 0.02843863 |
| A230084A06RIK | ILMN_1230144 | 0.43821397 | 0.79086678 | 0.01774597 | 0.58223073 | 0.00086964 | 0.0054444 |
| CXCL14 | ILMN_2659426 | 0.36725785 | 0.69848984 | 0.0227136 | 0.53631317 | 0.00146481 | 0.00400454 |
| TTC15 | ILMN_2502614 | 0.94781161 | 0.2793105 | 0.01461194 | 0.22510276 | 0.0204812 | 0.0003816 |
| TG737RPW | ILMN_2772847 | 0.76933501 | 0.54089135 | 0.01256087 | 0.3251282 | 0.00548678 | 0.032187 |
| AMBRA1 | ILMN_3048294 | 0.4436924 | 0.34743176 | 0.00444928 | 0.89777595 | 0.0232608 | 0.02156983 |
| control_ILMN_1378677 | ILMN_1378677 | 0.98344376 | 0.42084023 | 0.0119587 | 0.36340616 | 0.00643593 | 0.0105893 |
| PEF1 | ILMN_2643723 | 0.31905619 | 0.34992403 | 0.00231363 | 0.90266633 | 0.04569681 | 0.03244548 |
| D0HXS9928E | ILMN_2856628 | 0.9868439 | 0.24303218 | 0.01198025 | 0.19472258 | 0.0068983 | 0.0411307 |
| 9/2/2013 | ILMN_2756696 | 0.40369443 | 0.70675778 | 0.00624248 | 0.64127315 | 0.03249199 | 0.0116011 |
| OLFR519 | ILMN_2854265 | 0.64872481 | 0.83069642 | 0.04414921 | 0.43977727 | 0.00884027 | 0.04391917 |
| OLFR312 | ILMN_3161363 | 0.37093621 | 0.91228316 | 0.03416177 | 0.23883472 | 0.00061546 | 0.02052641 |
| AMBRA1 | ILMN_3124160 | 0.13009972 | 0.35347259 | 0.01710139 | 0.38735501 | 2.9826E−05 | 0.00021116 |
| COL4A1 | ILMN_2621643 | 0.42605115 | 0.74102405 | 0.02262109 | 0.58544953 | 0.00057902 | 0.00372274 |
| AI844366 | ILMN_2684007 | 0.82679825 | 0.75408665 | 0.01586452 | 0.95461649 | 0.00217068 | 0.00033328 |
| CAR6 | ILMN_1257323 | 0.61781612 | 0.3420742 | 0.01289426 | 0.0987578 | 0.00113957 | 0.0434174 |
| PLD1 | ILMN_2595408 | 0.6933796 | 0.45988626 | 0.02618266 | 0.18486916 | 0.00313609 | 0.04170925 |
| C230092H20RIK | ILMN_1233960 | 0.66357239 | 0.84781674 | 0.01253768 | 0.4360673 | 0.00394059 | 0.00171891 |
| GSTK1 | ILMN_2792924 | 0.40324013 | 0.70122688 | 0.02551463 | 0.55062775 | 0.00123964 | 0.00331843 |
| EPM2A | ILMN_2920454 | 0.87947439 | 0.80615461 | 0.01324935 | 0.95931201 | 0.0124013 | 0.00118461 |
| LOC383439 | ILMN_2539543 | 0.2952126 | 0.97743522 | 0.00256872 | 0.18462931 | 0.00013459 | 0.00034559 |
| C730048C13RIK | ILMN_2685022 | 0.28523072 | 0.20002172 | 0.00272924 | 0.97974436 | 0.03668703 | 0.01734854 |
| 2210010L05RIK | ILMN_2599306 | 0.79628945 | 0.72354551 | 0.01038968 | 0.94152678 | 0.00883444 | 0.0059479 |
| NAPA | ILMN_2865822 | 0.76548601 | 0.73543964 | 0.02191549 | 0.98861148 | 0.01018714 | 0.00217666 |
| GABRG1 | ILMN_1245521 | 0.81668262 | 0.70824678 | 0.0148667 | 0.87351206 | 0.01204321 | 0.01541948 |
| 4930471I01RIK | ILMN_1249396 | 0.28392524 | 0.5667521 | 0.0059659 | 0.54397727 | 0.00849713 | 0.00420644 |
| COBLL1 | ILMN_2782634 | 0.97106923 | 0.21183965 | 0.02557545 | 0.11963643 | 0.00505788 | 4.6218E−05 |
| CNTLN | ILMN_1240584 | 0.30681489 | 0.15860006 | 0.00774266 | 0.77825855 | 0.03721531 | 0.0339742 |
| TSNAXIP1 | ILMN_2881498 | 0.9924676 | 0.90236003 | 0.00711635 | 0.9143399 | 0.02610378 | 0.00620682 |
| A4GALT | ILMN_3009373 | 0.81161526 | 0.56933321 | 0.01848828 | 0.73650404 | 0.021082 | 0.02579472 |
| CHGA | ILMN_2814927 | 0.34839125 | 0.7266219 | 0.00963393 | 0.13811361 | 0.00058241 | 0.00918276 |
| SOX7 | ILMN_1234505 | 0.60939645 | 0.3749197 | 0.02100105 | 0.097519 | 0.02547775 | 0.0002976 |
| KIF17 | ILMN_2704842 | 0.39249283 | 0.70164439 | 0.01483827 | 0.51020186 | 0.04422883 | 0.00779006 |
| CDCA1 | ILMN_2755241 | 0.9154914 | 0.53536925 | 0.0386027 | 0.38918589 | 0.00874238 | 0.04952584 |
| TCF7L2 | ILMN_1231177 | 0.63686152 | 0.72325508 | 0.00931152 | 0.7890029 | 0.00503728 | 0.00048916 |
| OLFR506 | ILMN_2813771 | 0.21856348 | 0.1278011 | 0.02579809 | 0.93435701 | 0.00023607 | 2.4239E−05 |
| WNK2 | ILMN_1214402 | 0.93200737 | 0.63584187 | 0.03080328 | 0.66221401 | 0.01413717 | 0.03245347 |
| 8030455F02RIK | ILMN_2551098 | 0.59853823 | 0.19179375 | 0.0111058 | 0.41266041 | 0.01530089 | 0.04820028 |
| D430013G08RIK | ILMN_1241865 | 0.43949079 | 0.78980418 | 0.01370026 | 0.59445861 | 0.01747993 | 0.01248718 |
| NRXN1 | ILMN_2550623 | 0.44702512 | 0.62430211 | 0.00957581 | 0.70622754 | 0.00016695 | 0.00035354 |
| WIZ | ILMN_3095005 | 0.34305317 | 0.72411765 | 0.04555793 | 0.3711582 | 0.00166479 | 0.00306706 |
| BPHL | ILMN_2747284 | 0.73416126 | 0.61908049 | 0.00715923 | 0.36850034 | 0.01213128 | 0.00125811 |
| EG665903 | ILMN_2624523 | 0.86741803 | 0.99754893 | 0.01300844 | 0.82056514 | 0.00237114 | 0.00123216 |
| GPSN2 | ILMN_2606579 | 0.63692646 | 0.67878781 | 0.04674093 | 0.28470328 | 0.01272112 | 0.03760584 |
| V1RG5 | ILMN_2838831 | 0.82420699 | 0.98410461 | 0.01305096 | 0.80810588 | 0.01528723 | 0.09551877 |
| LOC668779 | ILMN_1248416 | 0.63537512 | 0.79619365 | 0.01938043 | 0.35121412 | 0.00881697 | 0.00113919 |
| AW538212 | ILMN_2688996 | 0.11535762 | 0.09428521 | 0.00400603 | 0.99292399 | 0.00010643 | 3.4806E−05 |
| D930029H24RIK | ILMN_1228871 | 0.47418638 | 0.47124874 | 0.00667883 | 0.98628298 | 0.00111202 | 0.00087401 |
| control_ILMN_1379309 | ILMN_1379309 | 0.77544733 | 0.60565037 | 0.01677348 | 0.81891206 | 0.03391372 | 0.04121776 |
| MFSD7C | ILMN_1248190 | 0.92106898 | 0.27325909 | 0.01727267 | 0.21669597 | 0.01901116 | 0.04929275 |
| EFNA5 | ILMN_2662536 | 0.3652588 | 0.55707841 | 0.00997123 | 0.10898185 | 0.00165732 | 0.04020568 |
| DMXL1 | ILMN_2429071 | 0.54914197 | 0.90451948 | 0.01438616 | 0.52202365 | 0.01177167 | 0.00187623 |
| LOC276803 | ILMN_2534316 | 0.16603663 | 0.54844716 | 0.0219723 | 0.27511942 | 0.00077337 | 0.00151001 |
| 2810043G13RIK | ILMN_2705440 | 0.81391852 | 0.24793276 | 0.00983154 | 0.32660096 | 0.00712057 | 0.01713054 |
| ABCF3 | ILMN_2677696 | 0.08078576 | 0.63645224 | 0.00192925 | 0.17132711 | 3.6728E−05 | 0.0011514 |
| 9830166F08RIK | ILMN_2586977 | 0.70658027 | 0.80333134 | 0.03743203 | 0.97975176 | 0.01119986 | 0.01679319 |
| PDE4B | ILMN_2574417 | 0.89763789 | 0.76767464 | 0.00644131 | 0.62596315 | 0.00122135 | 0.00391131 |
| GM378 | ILMN_1240049 | 0.61467321 | 0.42169273 | 0.02436088 | 0.82522224 | 0.03723516 | 0.02019999 |
| B230378H13RIK | ILMN_1237858 | 0.90912207 | 0.39640679 | 0.00978906 | 0.27459465 | 0.0053004 | 0.00011913 |
| 9530031H08RIK | ILMN_1246046 | 0.61512983 | 0.5241143 | 0.01321792 | 0.20960741 | 0.00438466 | 0.01828874 |
| 4930433I11RIK | ILMN_2599632 | 0.80008845 | 0.68076611 | 0.03193614 | 0.44763312 | 0.03817964 | 0.00324431 |
| CEBPE | ILMN_1220301 | 0.88001941 | 0.92607613 | 0.04789021 | 0.92909894 | 0.01339264 | 0.00930316 |
| MTA2 | ILMN_2595774 | 0.8956106 | 0.62296468 | 0.02801356 | 0.66195344 | 0.0018644 | 0.00226556 |
| APOC1 | ILMN_1228469 | 0.36613491 | 0.67118282 | 0.00583924 | 0.16428966 | 0.00159729 | 0.0193149 |
| NLRP5 | ILMN_2696556 | 0.36196978 | 0.72545148 | 0.02078351 | 0.48866047 | 0.00489731 | 0.00610399 |
| CYP2D26 | ILMN_2642975 | 0.79493857 | 0.70665534 | 0.00707043 | 0.49784891 | 0.00548722 | 0.01609651 |
| control_ILMN_1378609 | ILMN_1378609 | 0.98545756 | 0.69651186 | 0.01644739 | 0.64839753 | 0.00782567 | 0.01140479 |
| OLFR524 | ILMN_3160809 | 0.33912579 | 0.91057333 | 0.01465568 | 0.28077782 | 0.00233463 | 0.00521563 |
| LOC384689 | ILMN_1227050 | 0.56288345 | 0.91665544 | 0.0099089 | 0.49080727 | 0.00087983 | 0.00029031 |
| ADAT1 | ILMN_2977193 | 0.77440211 | 0.79237391 | 0.03035926 | 0.52872893 | 0.04858275 | 0.00661794 |
| CCDC15 | ILMN_1250884 | 0.62096729 | 0.78093474 | 0.02391441 | 0.36804853 | 0.00388307 | 0.01532034 |
| 3/8/2013 | ILMN_1242531 | 0.63936758 | 0.58731039 | 0.04723339 | 0.25275305 | 0.04544519 | 0.00757755 |
| C230094L10RIK | ILMN_1230165 | 0.95745392 | 0.97889682 | 0.01504046 | 0.97306341 | 0.00060298 | 0.00282531 |
| E130113E03RIK | ILMN_1235043 | 0.70634431 | 0.51296982 | 0.01980686 | 0.85243749 | 0.04411905 | 0.01857234 |
| C730029N23RIK | ILMN_1255622 | 0.64943846 | 0.80900421 | 0.00804317 | 0.41298409 | 0.00077399 | 0.00622362 |
| 3/6/2013 | ILMN_1242572 | 0.99421937 | 0.32189222 | 0.00830051 | 0.26400586 | 0.00342651 | 0.00011776 |
| LOC384155 | ILMN_2537186 | 0.94055232 | 0.89077988 | 0.04055628 | 0.94171882 | 0.02177121 | 0.0246912 |
| MTMR6 | ILMN_2687053 | 0.23687388 | 0.23005672 | 0.02671623 | 0.82719079 | 0.00086376 | 0.00122935 |
| 9790357_3511_RC | ILMN_2468115 | 0.50561161 | 0.55877521 | 0.03120211 | 0.16503585 | 0.00745488 | 0.04042447 |
| OLFR11 | ILMN_2619903 | 0.51966823 | 0.94983798 | 0.01570607 | 0.44153907 | 0.03630561 | 0.00269795 |
| 2510025K24RIK | ILMN_1235980 | 0.17034456 | 0.21438208 | 0.00229738 | 0.83886259 | 0.04982748 | 0.03586444 |
| SLC7A14 | ILMN_2679229 | 0.4024276 | 0.96217122 | 0.03248261 | 0.38012554 | 0.0048549 | 0.02265882 |
| 5830448L21RIK | ILMN_1254606 | 0.303375 | 0.1374351 | 0.01415928 | 0.8176851 | 0.00040346 | 7.2625E−06 |
| 4631434O19RIK | ILMN_1251169 | 0.82857753 | 0.44925318 | 0.03758512 | 0.25668358 | 0.01215362 | 0.03312515 |
| C230011P08RIK | ILMN_1258653 | 0.71366618 | 0.51631216 | 0.02426504 | 0.27992668 | 0.0156805 | 0.03212297 |
| OLFR483 | ILMN_2590828 | 0.44925085 | 0.80622851 | 0.04494222 | 0.24324882 | 0.0073063 | 0.02821443 |
| LOC382853 | ILMN_2536029 | 0.77875408 | 0.96843977 | 0.00975939 | 0.75198581 | 0.02153595 | 0.00272992 |
| SFTD6 | ILMN_1223358 | 0.57200419 | 0.74787399 | 0.01611525 | 0.66034764 | 0.00494207 | 0.00089435 |
| CBFA2T2 | ILMN_2752204 | 0.5662482 | 0.8989732 | 0.03228542 | 0.38785964 | 0.00475069 | 0.01302043 |
| LOC100045448 | ILMN_2776734 | 0.62132159 | 0.87870784 | 0.02123329 | 0.40374113 | 0.00294499 | 0.00314533 |
| OLFR535 | ILMN_2535079 | 0.32862437 | 0.16201386 | 0.00778611 | 0.8088199 | 0.03555645 | 0.02237539 |
| ATP2C1 | ILMN_1252963 | 0.49094217 | 0.65961812 | 0.04863474 | 0.17018373 | 0.00333117 | 0.04316958 |
| KLHDC8B | ILMN_1216669 | 0.97040393 | 0.74111168 | 0.02322535 | 0.70203261 | 0.02834159 | 0.02936116 |
| ADAMDEC1 | ILMN_1246807 | 0.94590728 | 0.9293539 | 0.02644931 | 0.98373974 | 0.01436855 | 0.00982324 |
| UNK | ILMN_2509928 | 0.17478517 | 0.75419036 | 0.03873043 | 0.05085358 | 0.00109399 | 0.03337033 |
| 5730460C07RIK | ILMN_1249562 | 0.87299005 | 0.36607511 | 0.0246919 | 0.26042351 | 0.03627524 | 0.00068321 |
| KLHL26 | ILMN_2647313 | 0.97277378 | 0.71962893 | 0.0261983 | 0.75480755 | 0.03900189 | 0.00295382 |
| LIG3 | ILMN_2828819 | 0.73599318 | 0.67552336 | 0.04595186 | 0.92486784 | 0.01276389 | 0.0118872 |
| PAM | ILMN_2626294 | 0.86894834 | 0.51715949 | 0.02535361 | 0.63556371 | 0.03167733 | 0.02768712 |
| V2R15 | ILMN_1242397 | 0.41219979 | 0.11478823 | 0.04367205 | 0.42360691 | 0.00428178 | 0.00027849 |
| 6430605CD3RIK | ILMN_2733534 | 0.85400928 | 0.88098485 | 0.01949348 | 0.67075129 | 0.01064616 | 0.00103457 |
| 8430429KD9RIK | ILMN_2441482 | 0.55336159 | 0.58108107 | 0.03598349 | 0.16703822 | 0.00189341 | 0.04391611 |
| C430004E15RIK | ILMN_2625047 | 0.36977921 | 0.98259491 | 0.01796861 | 0.26363911 | 0.00458685 | 0.00601476 |
| LOC244335 | ILMN_1238450 | 0.79674679 | 0.33055039 | 0.02094599 | 0.49108577 | 0.03096123 | 0.03299965 |
| DOXL2 | ILMN_2987657 | 0.26405581 | 0.09177888 | 0.03796562 | 0.65443938 | 0.00268763 | 0.00024394 |
| 4930554K12RIK | ILMN_2549226 | 0.90045387 | 0.71090699 | 0.04425982 | 0.79924246 | 0.03725714 | 0.03943207 |
| TGFBR2 | ILMN_1215171 | 0.80798761 | 0.44351108 | 0.03785897 | 0.60339963 | 0.01263689 | 0.00070953 |
| HPX | ILMN_2830333 | 0.66945363 | 0.4208039 | 0.03817863 | 0.80551597 | 0.02434854 | 0.00174409 |
| 2900052P03RIK | ILMN_1240268 | 0.43061293 | 0.79115638 | 0.01876921 | 0.56971594 | 0.03696311 | 0.01723125 |
| CPXCR1 | ILMN_2846457 | 0.86371414 | 0.9550009 | 0.03160792 | 0.77261816 | 0.01244667 | 0.00593288 |
| TAS2R106 | ILMN_2702928 | 0.91870736 | 0.6724644 | 0.01538494 | 0.58288995 | 0.01697577 | 0.02506084 |
| 4632404B13RIK | ILMN_1217530 | 0.81072013 | 0.83717324 | 0.03950811 | 0.96627929 | 0.04664843 | 0.04118925 |
| 1600012K10RIK | ILMN_1254674 | 0.95232816 | 0.60856678 | 0.00698531 | 0.53982436 | 0.00863333 | 0.00050927 |
| OLFR121 | ILMN_2979343 | 0.8832505 | 0.84790399 | 0.03234808 | 0.95369607 | 0.03233233 | 0.03873644 |
| 6130401J04RIK | ILMN_2604105 | 0.66759244 | 0.60660871 | 0.01258625 | 0.98036948 | 0.04634306 | 0.01250912 |
| CLPTM1L | ILMN_2681221 | 0.86491157 | 0.72209661 | 0.02504523 | 0.84097511 | 0.01423494 | 0.02311428 |
| 4732490P12 | ILMN_2453812 | 0.73774373 | 0.55857679 | 0.01338967 | 0.30618633 | 0.00541023 | 0.03370137 |
| LOC545289 | ILMN_1260380 | 0.94161903 | 0.60753873 | 0.02230898 | 0.51778884 | 0.01321341 | 0.04916193 |
| ATP6V0D1 | ILMN_2928320 | 0.69000763 | 0.72891399 | 0.0443055 | 0.38067515 | 0.01511458 | 0.02354026 |
| EG433144 | ILMN_2755355 | 0.87566173 | 0.80969571 | 0.04438241 | 0.95591322 | 0.04188234 | 0.01561656 |
| RAB11FIP5 | ILMN_3139693 | 0.99906414 | 0.21262481 | 0.04413508 | 0.17973027 | 0.03519113 | 0.0007083 |
| OLFR94 | ILMN_3160479 | 0.97600983 | 0.48305323 | 0.03747655 | 0.43339546 | 0.04591135 | 0.01129534 |
| LOC245453 | ILMN_1258169 | 0.98143674 | 0.59765716 | 0.04422655 | 0.53963945 | 0.02651704 | 0.01742043 |
| V1RD7 | ILMN_2446884 | 0.99095541 | 0.80565487 | 0.01895399 | 0.775485 | 0.01137664 | 0.00300505 |
| ANKRD35 | ILMN_1248776 | 0.70174763 | 0.15464086 | 0.03786988 | 0.28227631 | 0.01573414 | 0.00060094 |
| SETD6 | ILMN_2983669 | 0.33252567 | 0.83314552 | 0.04795023 | 0.29627711 | 0.00036932 | 0.00512599 |
| SQLE | ILMN_2573600 | 0.93659847 | 0.88539347 | 0.03256569 | 0.8004337 | 0.01646469 | 0.02425755 |
| A330004N04RIK | ILMN_1217986 | 0.67141686 | 0.41019524 | 0.02744773 | 0.72871997 | 0.01111549 | 0.00135098 |
| USP22 | ILMN_2494618 | 0.47531038 | 0.218617 | 0.02422803 | 0.65853923 | 0.00221926 | 0.00016059 |
| control_ILMN_1378808 | ILMN_1378808 | 0.79181076 | 0.79149038 | 0.02044898 | 0.98505154 | 0.03587143 | 0.02032572 |
| AK122498 | ILMN_2524245 | 0.85736966 | 0.94151199 | 0.02377537 | 0.77239483 | 0.01378398 | 0.01117137 |
| LOC381546 | ILMN_1248921 | 0.79668012 | 0.70699032 | 0.04506863 | 0.43926623 | 0.00829223 | 0.02767371 |
| ATP7B | ILMN_2731102 | 0.6249321 | 0.37736846 | 0.0129853 | 0.77700164 | 0.01386252 | 0.00133534 |
| RBM41 | ILMN_2719569 | 0.93448297 | 0.64047478 | 0.04161783 | 0.52393995 | 0.02134418 | 0.00399331 |
| D610007L01RIK | ILMN_1232042 | 0.85594071 | 0.43791688 | 0.02856732 | 0.28912260 | 0.02809339 | 0.00264701 |
| 2610528H13RIK | ILMN_1212831 | 0.39305556 | 0.95996982 | 0.03970997 | 0.30048283 | 0.00369356 | 0.00979345 |
| D030018H12RIK | ILMN_1215334 | 0.84407243 | 0.81541907 | 0.03183662 | 0.98023643 | 0.04253257 | 0.03735335 |
| CDH12 | ILMN_1253923 | 0.59433128 | 0.77701969 | 0.04166861 | 0.32554775 | 0.04535074 | 0.00743212 |
| E430014N10RIK | ILMN_1240823 | 0.34359038 | 0.61481686 | 0.02121754 | 0.53341668 | 0.0009554 | 0.00206857 |
| LRRC17 | ILMN_2511456 | 0.95514688 | 0.83357486 | 0.02908474 | 0.76599237 | 0.01433455 | 0.02248209 |
| 4931417G12RIK | ILMN_2710573 | 0.91281571 | 0.38909731 | 0.02420117 | 0.28646512 | 0.02132946 | 0.00096049 |
| OLFR615 | ILMN_1231317 | 0.54364931 | 0.80154308 | 0.0273447 | 0.32662044 | 0.01457818 | 0.02687296 |
| LOC631010 | ILMN_1240227 | 0.94686881 | 0.26102934 | 0.03305513 | 0.26204501 | 0.02373213 | 0.00072505 |
| PPP1CA | ILMN_2691613 | 0.09795108 | 0.59506137 | 0.02504734 | 0.20664497 | 0.00014769 | 0.00232552 |
| E130009J12RIK | ILMN_2985193 | 0.47477677 | 0.81044479 | 0.03110782 | 0.63617091 | 0.00487679 | 0.02678105 |
| CD209A | ILMN_2617210 | 0.22035646 | 0.46771383 | 0.04211656 | 0.51778746 | 0.00339062 | 0.00413649 |
| TFPI | ILMN_2691522 | 0.47180301 | 0.91902103 | 0.03419503 | 0.43496331 | 0.00859842 | 0.01560756 |
| 5730530J16RIK | ILMN_1249981 | 0.5888761 | 0.67822628 | 0.01711406 | 0.84804259 | 0.04732222 | 0.02349276 |
| EG666609 | ILMN_2742647 | 0.46040682 | 0.48129977 | 0.02438815 | 0.91160425 | 0.03812319 | 0.03481446 |
| VPS13D | ILMN_1230695 | 0.99703152 | 0.92779661 | 0.02251575 | 0.93072108 | 0.02874602 | 0.00736329 |
| BCL11A | ILMN_1234185 | 0.54620214 | 0.57922704 | 0.0125649 | 0.92397804 | 0.00460099 | 0.00387138 |
| PVRL2 | ILMN_1218242 | 0.77166479 | 0.49252111 | 0.03288426 | 0.25489684 | 0.01005689 | 0.03917443 |
| GM392 | ILMN_1240780 | 0.48222112 | 0.81340913 | 0.02162921 | 0.28763551 | 0.00084466 | 0.01753709 |
| 3110012M05RIK | ILMN_1219662 | 0.97368985 | 0.64870795 | 0.03780242 | 0.64874884 | 0.027784 | 0.0075749 |
| 6330437I11RIK | ILMN_1228346 | 0.54055553 | 0.42081215 | 0.0385954 | 0.84463922 | 0.01048267 | 0.00565422 |
| 4921537F17RIK | ILMN_1247508 | 0.61998523 | 0.86883509 | 0.04136706 | 0.46063606 | 0.00876015 | 0.03457809 |
| MAK10 | ILMN_2612403 | 0.10085284 | 0.78633106 | 0.03440787 | 0.09093 | 6.9925E−05 | 0.00808975 |
| FTS | ILMN_1219649 | 0.25096355 | 0.50514838 | 0.02634812 | 0.5904826 | 0.00373255 | 0.00641831 |
| PHXR5 | ILMN_2669458 | 0.17900286 | 0.17469022 | 0.02196101 | 0.95767368 | 0.00036712 | 0.00086747 |
| genericLS | ILMN_2563949 | 0.17385044 | 0.22134332 | 0.04986018 | 0.71296708 | 0.0003577 | 0.00036446 |
| DEFCR-R51 | ILMN_2524936 | 0.43135334 | 0.94370885 | 0.04067994 | 0.39706896 | 0.00628876 | 0.02257305 |
| TSSK6 | ILMN_2650022 | 0.74044916 | 0.8955475 | 0.02682062 | 0.82887811 | 0.0195886 | 0.02870796 |
| LOC100044218 | ILMN_1245604 | 0.62060671 | 0.93959659 | 0.03808725 | 0.61900798 | 0.00370233 | 0.01736491 |
| GULO | ILMN_2631948 | 0.0975262 | 0.91952112 | 0.04013288 | 0.05534954 | 0.00032512 | 0.01308216 |
| LOC381636 | ILMN_1232330 | 0.42177934 | 0.26930523 | 0.04263458 | 0.76923218 | 0.00561836 | 0.00181091 |
| 1810019D21RIK | ILMN_1227679 | 0.28059575 | 0.2048988 | 0.04144693 | 0.89865913 | 0.0049656 | 0.00175637 |
| LOC100039649 | ILMN_1215436 | 0.90624752 | 0.99641741 | 0.04630688 | 0.89063302 | 0.03849951 | 0.03837077 |
| 2900097C17RIK | ILMN_1227592 | 0.56873815 | 0.42250402 | 0.03414773 | 0.83287881 | 0.00673111 | 0.00353241 |
| SEC11A | ILMN_2652936 | 0.65017753 | 0.19928299 | 0.04565721 | 0.3753999 | 0.00777675 | 0.00041973 |
| BOC | ILMN_1227184 | 0.83379882 | 0.63931653 | 0.04618015 | 0.42702865 | 0.01169619 | 0.04374422 |
| EG224576 | ILMN_1215517 | 0.14284265 | 0.38836352 | 0.04273586 | 0.59084228 | 0.00181023 | 0.00657723 |
| E030045A12RIK | ILMN_2572276 | 0.7533502 | 0.25562274 | 0.03987607 | 0.39803578 | 0.03931825 | 0.00635341 |
| TCF7L2 | ILMN_2704212 | 0.40659811 | 0.2024417 | 0.0385675 | 0.7023388 | 0.00714442 | 0.00123425 |
| 3732412D22RIK | ILMN_1216840 | 0.81984654 | 0.70192537 | 0.03267402 | 0.91525163 | 0.03935182 | 3.01007019 |
| SCL0002508.1_73 | ILMN_2460618 | 0.07304885 | 0.40335908 | 0.03500519 | 0.24340242 | 0.00064943 | 0.00332745 |
| D030002E05RIK | ILMN_2443894 | 0.98716805 | 0.30863962 | 0.04412443 | 0.27596309 | 0.04853151 | 0.00225048 |

| | | AVG | | | | | | |
|---|---|---|---|---|---|---|---|---|
| | AVG | Reference | | | | | | |
| Gene | Medium | Standard | AVG GA | AVG generic | | | | |
| 2610027L16RIK | 7.383123333 | 7.472973684 | 7.497494118 | 7.814259091 | | | | |
| F830002E14RIK | 7.757403333 | 7.689255263 | 7.754048529 | 8.152786364 | | | | |
| TPSAB1 | 6.547536667 | 6.53815 | 6.616708824 | 6.831309091 | | | | |
| G6PC3 | 7.870143333 | 7.920968421 | 7.948063235 | 8.117036364 | | | | |
| MGAT4B | 6.956693333 | 7.005913158 | 7.019420588 | 7.194036364 | Pattern Represented by Data: | | | |
| GM962 | 6.877433333 | 6.909928947 | 6.952055882 | 7.109163636 | | | | x |
| SCMH1 | 7.173013333 | 7.179326315 | 7.202801471 | 7.402677273 | x | x | x | |
| SLC25A39 | 8.332893333 | 8.396894737 | 8.393611765 | 8.557713636 | | | | |
| OLFM1 | 6.3626 | 6.397260526 | 6.422316176 | 6.583404545 | Medium | Reference | GA | generic |
| SNCA | 6.82406 | 6.8204 | 6.823285294 | 7.034854545 | | | | |
| SNCA | 7.675763333 | 7.688563158 | 7.719423529 | 7.885086364 | | | | |
| 2310003H01RIK | 9.07639 | 9.134994737 | 9.099767647 | 9.279345455 | | | | |
| MARCKS | 9.683343333 | 9.709323684 | 9.731372059 | 9.883690909 | | | | |
| LMAN2L | 8.09457 | 8.121555263 | 8.103702941 | 8.290409091 | | | | |
| PDHB | 6.709056667 | 6.794373684 | 6.787814706 | 6.90445 | | | | |
| 1300001I01RIK | 7.676173333 | 7.747807895 | 7.714445588 | 7.870945455 | | | | |
| MARCH2 | 8.727983333 | 8.768297368 | 8.797601471 | 8.922431818 | | | | |
| REPIN1 | 6.464103333 | 6.508721053 | 6.515754412 | 6.656413636 | | | | |
| ALG12 | 6.68395 | 6.743931579 | 6.696648529 | 6.871754545 | | | | |
| SMARCAL1 | 8.057763333 | 8.091636842 | 8.072047059 | 8.24295 | | | | |
| TRIP10 | 7.159423333 | 7.17705 | 7.219839706 | 7.344527273 | | | | |
| PHLPP | 7.11814 | 7.179828947 | 7.143044118 | 7.301213636 | | | | |
| WDR6 | 8.723316667 | 8.696534211 | 8.763451471 | 8.904881818 | | | | |
| TRIM28 | 11.25953333 | 11.29731053 | 11.31465882 | 11.44073182 | | | | |
| 1110039B18RIK | 8.343063333 | 8.346705263 | 8.355070588 | 8.522913636 | | | | |
| CHMP2A | 9.846833333 | 9.916607895 | 9.924004412 | 10.02553636 | | | | |
| PIGT | 8.51204 | 8.501307895 | 8.530822059 | 8.690481818 | | | | |
| LOC100044636 | 6.805556667 | 6.867765789 | 6.857189706 | 6.983836364 | | | | |
| PLEKHO2 | 9.02316 | 9.027347368 | 9.057944118 | 9.201381818 | | | | |
| EHBP1L1 | 7.960713333 | 7.962668421 | 8.019623529 | 8.138863636 | | | | |
| ACSL1 | 7.0019 | 7.036060526 | 6.999769118 | 7.177977273 | | | | |
| 2310016E02RIK | 7.304916667 | 7.353897368 | 7.379801471 | 7.479331818 | | | | |
| ZFP42 | 5.924646667 | 5.978389474 | 5.971730882 | 6.098336364 | | | | |
| LOC100044576 | 7.004646667 | 7.055815789 | 7.054444118 | 7.175572727 | | | | |
| CLECSF12 | 6.01422 | 6.045115789 | 6.065151471 | 6.183786364 | | | | |
| RFFL | 6.479006667 | 5.483426316 | 6.499785235 | 6.647177273 | | | | |
| MEIS1 | 6.53368 | 6.576773684 | 6.579920588 | 5.701572727 | | | | |
| EI24 | 8.8673 | 8.904455263 | 8.911220588 | 9.035086364 | | | | |
| DCP1A | 8.658053333 | 8.723913158 | 8.709516176 | 8.825331818 | | | | |
| 9330147J08RIK | 6.87715 | 6.936536842 | 6.934904412 | 7.044290909 | | | | |
| NFS1 | 7.170636667 | 7.236936842 | 7.236829412 | 7.337427273 | | | | |
| YWHAG | 6.453236667 | 6.521060526 | 6.511844118 | 6.62 | | | | |
| FMNL3 | 6.104096667 | 6.130421053 | 6.1567 | 6.2686 | | | | |
| ARF3 | 8.823646667 | 8.876202632 | 8.888575 | 8.987454545 | | | | |
| CSF2RA | 7.395346667 | 7.397218421 | 7.364389706 | 7.558909091 | | | | |
| DHRSX | 7.434513333 | 7.457857895 | 7.429736765 | 7.596972727 | | | | |
| LOC100046035 | 5.793826667 | 5.853584211 | 5.868417647 | 5.956231818 | | | | |
| 2610019A05RIK | 7.04731 | 7.035371053 | 7.0915 | 7.209186364 | | | | |
| SCHIP1 | 6.35611 | 6.325936842 | 6.336330882 | 6.517436364 | | | | |
| 4632417N05RIK | 6.254436667 | 6.288605263 | 6.299548529 | 6.415672727 | | | | |
| DORZ1 | 6.848643333 | 6.864202632 | 6.876232353 | 7.009645455 | | | | |
| CPNE2 | 6.59805 | 6.627957895 | 6.619660294 | 6 758777273 | | | | |
| GPD1L | 5.962863333 | 6.012323684 | 6.011677941 | 6.122713636 | | | | |
| BC018242 | 6.208366667 | 6.270368421 | 6.249745588 | 6.367659091 | | | | |
| C230090D14 | 5.947733333 | 5.998884211 | 5.99155 | 6.106377273 | | | | |
| TSPAN31 | 6.763363333 | 6.787294737 | 6.736430882 | 6.921695455 | | | | |
| POLB | 6.965993333 | 7.0099 | 7.016457353 | 7.124222727 | | | | |
| SIAH1B | 6.37228 | 6.436939474 | 6.393475 | 6.528490909 | | | | |
| LOC383036 | 6.41209 | 6.474181579 | 6.472569118 | 6.567927273 | | | | |
| LYNX1 | 6.817296667 | 6.848018421 | 6.870923529 | 6.972218182 | | | | |
| ZFP41 | 7.414063333 | 7.450618421 | 7.419467647 | 7.568736364 | | | | |
| TSPAN33 | 6.582713333 | 6.613352632 | 6.641277941 | 6.735368182 | | | | |
| MEGF8 | 7.129433333 | 7.184897368 | 7.159761765 | 7.281790909 | | | | |
| GPAA1 | 7.391883333 | 7.385360526 | 7.441967647 | 7.544040909 | | | | |
| SLC7A8 | 6.23386 | 6.259007895 | 6.268110294 | 6.384822727 | | | | |
| RHOF | 9.890486667 | 9.899973684 | 9.947642647 | 10.04121364 | | | | |
| 2810453I06RIK | 8.4568 | 8.506107895 | 8.506408824 | 8.607172727 | | | | |
| PCDHAC1 | 5.65515 | 5.716521053 | 5.689845588 | 5.804577273 | | | | |
| SCN1B | 5.966606667 | 5.975315789 | 5.969526471 | 6.115209091 | | | | |
| CDC42BPB | 7.155186667 | 7.118955263 | 7.152523529 | 7.303131818 | | | | |
| BC037704 | 7.20203 | 7.214631579 | 7.193013235 | 7.3494 | | | | |
| ADAR | 8.79254 | 8.859055263 | 8.857651471 | 8.939627273 | | | | |
| UACA | 6.05639 | 6.087271053 | 6.099835294 | 6.203304545 | | | | |
| HIST2H2AA1 | 6.169153333 | 6.226644737 | 6.224104412 | 6.315718182 | | | | |
| PRDX2 | 11.56810333 | 11.61388947 | 11.61041618 | 11.71427273 | | | | |
| TFRC | 9.730386667 | 9.798923684 | 9.788595588 | 9.876177273 | | | | |
| MTAP1S | 8.951756667 | 8.937260526 | 8.991608824 | 9.097072727 | | | | |
| D11WSU47E | 7.024173333 | 7.063092105 | 7.067933824 | 7.168595455 | | | | |
| ADPRHL2 | 6.74877 | 6.730905263 | 6.782579412 | 6.892372727 | | | | |
| SYMPK | 8.383586667 | 8.397528947 | 8.402226471 | 8.52715 | | | | |
| GYPA | 6.934123333 | 6.9204 | 6.897686765 | 7.0776 | | | | |
| KLHDC3 | 7.81348 | 7.822594737 | 7.853058824 | 7.955713636 | | | | |
| 3110070M22RIK | 6.120523333 | 6.180081579 | 6.160217647 | 6.262686364 | | | | |
| TRUB1 | 8.182053333 | 8.2163 | 8.230364706 | 8.323472727 | | | | |
| AKT2 | 5.950246667 | 5.944173684 | 5.919175 | 6.091436364 | | | | |
| RSC1A1 | 7.647993333 | 7.659592105 | 7.697304412 | 7.787868182 | | | | |
| FRY | 5.699756667 | 5.725107895 | 5.740185294 | 5.839477273 | | | | |
| TJAP1 | 8.189586667 | 8.215047368 | 8.167745588 | 8.328781818 | | | | |
| CNGA4 | 5.99744 | 6.028623684 | 6.004719118 | 6.136186364 | | | | |
| SGSM3 | 7.17779 | 7.212339474 | 7.217123529 | 7.316372727 | | | | |
| 9430078K10RIK | 6.076453333 | 6.095631579 | 6.124530882 | 6.214954545 | | | | |
| SLC26A6 | 6.420846667 | 6.4567 | 6.453741176 | 6.5591 | | | | |
| LYSMD4 | 6.801406667 | 6.835131579 | 6.812473529 | 6.939245455 | | | | |
| PCBD2 | 10.26506 | 10.27223684 | 10.32058088 | 10.40271364 | | | | |
| UAP1 | 8.167053333 | 8.216581579 | 8.167673529 | 8.304695455 | | | | |
| RNF40 | 6.104593333 | 6.138042105 | 6.147527941 | 6.241636364 | | | | |
| LOC277049 | 5.871153333 | 5.901181579 | 5.915414706 | 6.006836364 | | | | |
| LOC100047603 | 6.163923333 | 6.187207895 | 6.184048529 | 6.299472727 | | | | |
| BCL2L12 | 6.95393 | 6.980221053 | 6.997483824 | 7.088572727 | | | | |
| HMGCL | 7.923806667 | 7.963534211 | 7.958 | 8.0583 | | | | |
| TRPV4 | 5.866016667 | 5.907376316 | 5.895151471 | 5.999986364 | | | | |
| 9030624J02RIK | 9.494316667 | 9.503005263 | 9.516620588 | 9.627690909 | | | | |
| 11110020P15RIK | 12.83764667 | 12.8778 | 12.87814853 | 12.97065 | | | | |
| SNCA | 5.838986667 | 5.871242105 | 5.874923529 | 5.971345455 | | | | |
| 9130011J15RIK | 8.423743333 | 8.460226316 | 8.444064706 | 8.556095455 | | | | |
| ATP9B | 5.876853333 | 5.884023684 | 5.920125 | 6.008981818 | | | | |
| KCNC3 | 5.950906667 | 5.980818421 | 5.961808824 | 6.082136364 | | | | |
| LOC100045460 | 5.620116667 | 5.600318421 | 5.645004412 | 5.749863636 | | | | |
| MAGEE2 | 5.760393333 | 5.796631579 | 5.808379412 | 5.889972727 | | | | |
| LOC100047194 | 5.874716667 | 5.918439474 | 5.920526471 | 6.00425 | | | | |
| MS4A7 | 6.47951 | 6.501352632 | 6.505692647 | 6.608881918 | | | | |
| EFCAB6 | 5.580346667 | 5.591255263 | 5.609620588 | 5.709145455 | | | | |
| CEBPE | 6.161536667 | 6.170128947 | 6.20425 | 6.289590909 | | | | |
| LOC100046781 | 6.09563 | 6.110731579 | 6.088780882 | 6.223009091 | | | | |
| C920004C08RIK | 11.32737 | 11.35911842 | 11.38204412 | 11.45473636 | | | | |
| TNXB | 5.663876667 | 5.699578947 | 5.702611765 | 5.790745455 | | | | |
| ADAL | 6.324213333 | 6.367818421 | 6.366766176 | 6.450545455 | | | | |
| INTS7 | 9.293426667 | 9.331626316 | 9.311188235 | 9.41915 | | | | |
| FOXP4 | 6.264146667 | 6.276007895 | 6.284380882 | 6.389745455 | | | | |
| Ageneric5L | 8.109116667 | 8.115305263 | 8.106152941 | 8.234490909 | | | | |
| CD44 | 5.908876667 | 5.950365789 | 5.953 | 6.034186364 | | | | |
| ZMAT3 | 7.620363333 | 7.659939474 | 7.615426471 | 7.745318182 | | | | |
| PRPF3 | 9.9884 | 10.01081053 | 10.04008088 | 10.11331818 | | | | |
| RPUSD3 | 6.720803333 | 6.761342105 | 6.755892647 | 6.845586364 | | | | |
| ZBTB6 | 5.991976667 | 6.036765789 | 6.028576471 | 6.116695455 | | | | |
| MAGEA9 | 5.76786 | 5.7748 | 5.817098529 | 5.892554545 | | | | |
| PTPRF | 5.6626 | 5.695144737 | 5.709207353 | 5.786977273 | | | | |
| NSF | 9.924636667 | 9.963881579 | 9.949961765 | 10.04892273 | | | | |
| SARDH | 5.826933333 | 5.817136842 | 5.836519118 | 5.949331818 | | | | |
| XPO5 | 7.6071 | 7.625202632 | 7.592019118 | 7.729186364 | | | | |
| VGLL2 | 5.813743333 | 5.846973684 | 5.852779412 | 5.935795455 | | | | |
| HSD3B2 | 5.780146667 | 5.805763158 | 5.775658824 | 5.9017 | | | | |
| CAPZB | 5.8003 | 5.843094737 | 5.853869118 | 5.921709091 | | | | |
| PKIB | 5.772683333 | 5.783521053 | 5.802783824 | 5.894004545 | | | | |
| A230005A19RIK | 5.803403333 | 5.79155 | 5.830422059 | 5.924704545 | | | | |
| USP10 | 6.42393 | 6.434371053 | 6.431577941 | 6.544827273 | | | | |
| NCDN | 8.381273333 | 8.373278947 | 8.408173529 | 8.501877273 | | | | |
| SYN1 | 5.950843333 | 5.935671053 | 5.984697059 | 6.071295455 | | | | |
| ARVCF | 5.718253333 | 5.738763158 | 5.726697059 | 5.838531818 | | | | |
| SMG7 | 9.624156667 | 9.658326316 | 9.655355882 | 9.744204545 | | | | |
| 5730472N09RIK | 7.63479 | 7.660244737 | 7.648992647 | 7.754604545 | | | | |
| ZMIZ1 | 7.174863333 | 7.131644737 | 7.099030882 | 7.294513636 | | | | |
| ZFP162 | 7.66155 | 7.620365789 | 7.651876471 | 7.781118182 | | | | |
| DUS2L | 6.93203 | 6.926063158 | 6.953073529 | 7.050854545 | | | | |
| BZRPL1 | 6.187326667 | 6.206431579 | 6.203076471 | 6.306068182 | | | | |
| ARFGAP2 | 9.42326 | 9.430544737 | 9.428569118 | 9.541945455 | | | | |
| 9330183F02RIK | 5.949093333 | 5.973960526 | 5.98625 | 6.067668182 | | | | |
| DBF4 | 8.57731 | 8.598042105 | 8.569597059 | 8.695836364 | | | | |
| ATG3 | 5.596373333 | 5.578073684 | 5.592863235 | 5.714631818 | | | | |
| ISCA1 | 9.365886207 | 9.401326316 | 9.377892647 | 9.484090909 | | | | |
| 4930544L04RIK | 5.835473333 | 5.881492105 | 5.873111765 | 5.953290909 | | | | |
| 2610209A20RIK | 5.954303333 | 5.9957 | 6.007576471 | 6.072068182 | | | | |
| 2810046M22RIK | 5.965796667 | 5.974528947 | 5.994625 | 6.08355 | | | | |
| DHDH | 7.466183333 | 7.457073684 | 7.453639706 | 7.583509091 | | | | |
| ALOX15 | 5.948553333 | 5.970678947 | 5.967667647 | 6.065718182 | | | | |
| RNF183 | 5.951953333 | 5.990247368 | 5.994061765 | 6.069068182 | | | | |
| LOC385989 | 5.9626 | 6.003389474 | 5.996039706 | 6.079709091 | | | | |
| RBPMS | 6.082373333 | 6.081173684 | 6.068086765 | 6.199422727 | | | | |
| MTRR | 6.4451 | 6.491657895 | 6.473463235 | 6.562140909 | | | | |
| 5930400G23RIK | 5.852196667 | 5.880894737 | 5.875044118 | 5.968995455 | | | | |
| CD151 | 7.20143 | 7.205263158 | 7.185635294 | 7.318127273 | | | | |
| LOC380636 | 5.523523333 | 5.565797368 | 5.565647059 | 5.639413636 | | | | |
| ITFG2 | 8.126836667 | 8.117552632 | 8.148073529 | 8.242622727 | | | | |
| PALM | 6.404466667 | 6.3666 | 6.420661765 | 6.519804545 | | | | |
| ZFP563 | 5.780793333 | 5.821705263 | 5.808876471 | 5.894754545 | | | | |
| LOC382813 | 5.826046667 | 5.796476316 | 5.785617647 | 5.939763636 | | | | |
| BC017612 | 7.401256667 | 7.409202632 | 7.393575 | 7.514886364 | | | | |
| CBARA1 | 7.51929 | 7.512352632 | 7.516942647 | 7.632227273 | | | | |
| COQ9 | 8.24214 | 8.258726316 | 8.284261765 | 8.355045455 | | | | |
| USP36 | 8.529063333 | 8.552176316 | 8.499982353 | 8.641940909 | | | | |
| 1700063H04RIK | 5.77347 | 5.789544737 | 5.819770588 | 5.886254545 | | | | |
| JPH1 | 5.62534 | 5.656123684 | 5.671236765 | 5.737745455 | | | | |
| CLEC4A3 | 6.565526667 | 6.496394737 | 6.547061765 | 6.6777 | | | | |
| R74862 | 6.187246667 | 6.161294737 | 6.214616176 | 6.299313636 | | | | |
| LOC332300 | 6.22264 | 6.254697368 | 6.24265 | 6.334690909 | | | | |
| SPEN | 6.88912 | 6.833476316 | 6.890566176 | 7.000954545 | | | | |
| CHRM2 | 5.612146667 | 5.627805263 | 5.589489706 | 5.723913636 | | | | |
| PSG22 | 5.674963333 | 5.713052632 | 5.693373529 | 5.786663636 | | | | |
| E330037I15RIK | 6.253253333 | 6.295521053 | 6.256348529 | 6.364609091 | | | | |
| SCAVENGER RECEPTOR | 6.180263333 | 6.122484211 | 6.160936765 | 6.291545455 | | | | |
| UBE2E2 | 6.42196 | 6.408394737 | 6.420322059 | 6.533227273 | | | | |
| PALLD | 5.6114 | 5.651594737 | 5.640960294 | 5.721868182 | | | | |
| REXO1 | 6.846273333 | 6.8226 | 6.853654412 | 6.956654545 | | | | |
| 9330104G04RIK | 5.66529 | 5.685615789 | 5.709814706 | 5.775472727 | | | | |
| PKIA | 6.33296 | 6.318071053 | 6.356127941 | 6.442677273 | | | | |
| 1200003C05RIK | 9.058593333 | 9.076478947 | 9.102913235 | 9.168109091 | | | | |
| C130040D06RIK | 5.67662 | 5.685352632 | 5.710569118 | 5.78585 | | | | |
| MOCO5 | 6.452413333 | 6.442544737 | 6.424154412 | 6.561495455 | | | | |
| PCOLN3 | 7.14645 | 7.129347368 | 7.171211765 | 7.2555 | | | | |
| DACH1 | 5.84774 | 5.876168421 | 5.873570588 | 5.956586364 | | | | |
| B930062B16RIK | 5.9928 | 6.020252632 | 6.038070588 | 6.1015 | | | | |
| UGP2 | 7.75135 | 7.759273684 | 7.767085294 | 7.859927273 | | | | |
| RALGPS2 | 8.849346667 | 8.85575 | 8.862479412 | 8.957890909 | | | | |
| MFSD7A | 5.942556667 | 5.962721053 | 5.960813235 | 6.050986364 | | | | |
| KREMEN2 | 6.228356667 | 6.229486842 | 6.222888235 | 6.336777273 | | | | |
| LOC223628 | 5.593906667 | 5.627542105 | 5.597889706 | 5.702255091 | | | | |
| RASAL2 | 5.9777 | 6.015571053 | 6.006291176 | 6.085986364 | | | | |
| OLFR1155 | 5.764156667 | 5.776971053 | 5.789191176 | 5.872309091 | | | | |
| 1700021P22RIK | 6.012316667 | 6.023542105 | 6.037217647 | 6.120245455 | | | | |
| 9430088N01RIK | 6.635966667 | 6.672292105 | 6.673269118 | 6.743536364 | | | | |
| MKNK1 | 6.082133333 | 6.116702632 | 6.113097059 | 6.189422727 | | | | |
| FAHD1 | 7.16378 | 7.178176316 | 7.176764706 | 7.270995455 | | | | |
| 4930432K21RIK | 6.587463333 | 6.603065789 | 6.592614706 | 6.694659091 | | | | |
| KRTCAP2 | 12.50929 | 12.52731053 | 12.5338 | 12.61621818 | | | | |
| LY96 | 7.739873333 | 7.721665789 | 7.768026471 | 7.846718182 | | | | |
| TAF3 | 7.20446 | 7.230068421 | 7.199411765 | 7.310913636 | | | | |
| 1200011M11RIK | 6.336033333 | 6.345536842 | 6.321513235 | 6.442459091 | | | | |
| 4930430K04RIK | 5.489846667 | 5.518073684 | 5.499239706 | 5.596013636 | | | | |
| FGR | 6.94495 | 6.947236842 | 6.971538235 | 7.051018182 | | | | |
| 5830457O10RIK | 9.167493333 | 9.133463158 | 9.144754412 | 9.273427273 | | | | |
| FBXL21 | 5.61257 | 5.652573684 | 5.656242647 | 5.718277273 | | | | |
| BC031853 | 6.96577 | 6.904671053 | 6.951757353 | 7.071477273 | | | | |
| LOC546088 | 5.760926667 | 5.771278947 | 5.781745588 | 5.866559091 | | | | |
| control_ILMN_1378661 | 5.73423 | 5.717705263 | 5.718148529 | 5.839836364 | | | | |
| HTATIP2 | 8.76101 | 8.781044737 | 8.787325 | 8.866609091 | | | | |
| CENTD3 | 7.796676667 | 7.763389474 | 7.760257353 | 7.902195455 | | | | |
| DNAHC17 | 5.836333333 | 5.843681579 | 5.837757353 | 5.941763636 | | | | |
| 5330403O16RIK | 5.679523333 | 5.713576316 | 5.716461765 | 5.784904545 | | | | |
| CCL2 | 5.806643333 | 5.794926316 | 5.833814706 | 5.911954545 | | | | |
| C230062L16RIK | 6.18329 | 6.191994737 | 6.189144118 | 6.288490909 | | | | |
| 1700029G01RIK | 6.738916667 | 6.749055263 | 6.737847059 | 6.844031818 | | | | |
| 9330158H04RIK | 5.684246667 | 5.697526316 | 5.698854412 | 5.789281818 | | | | |
| ENAH | 5.894096667 | 5.941060526 | 5.919310294 | 5.999045455 | | | | |
| SLC13A5 | 5.689653333 | 5.687397368 | 5.704489706 | 5.794518182 | | | | |
| 6720407G21RIK | 5.56161 | 5.557984211 | 5.543336765 | 5.666440909 | | | | |
| BTRC | 7.959056667 | 7.927918421 | 7.945702941 | 8.063472727 | | | | |
| COTL1 | 12.2261 | 12.23490263 | 12.24276029 | 12.33050455 | | | | |
| NEK4 | 5.931456667 | 5.927926316 | 5.968935294 | 6.035831818 | | | | |
| 6430709H04RIK | 5.620953333 | 5.625018421 | 5.641717647 | 5.725077273 | | | | |
| TCEAL5 | 5.74579 | 5.744702632 | 5.768248529 | 5.849777273 | | | | |
| NFE2L1 | 9.749116667 | 9.772821053 | 9.752560294 | 9.852936364 | | | | |
| THOC1 | 8.791263333 | 8.7994 | 8.813451471 | 8.894681818 | | | | |
| CTNNA3 | 5.677696667 | 5.704381579 | 5.700572059 | 5.780836364 | | | | |
| 9430003J03RIK | 6.223606667 | 6.235028947 | 6.250230882 | 6.326727273 | | | | |
| NAPRT1 | 6.49092 | 6.492510526 | 6.460433824 | 6.593895455 | | | | |
| 5YNGR1 | 5.925626667 | 5.922781579 | 5.953997059 | 6.028540909 | | | | |
| LOC380723 | 5.8033 | 5.824481579 | 5.811860294 | 5.906168182 | | | | |
| D130017N08RIK | 6.040376667 | 6.081463158 | 6.061413235 | 6.142936364 | | | | |
| LOC677528 | 9.500013333 | 9.476392105 | 9.497989706 | 9.602481818 | | | | |
| BCL2L2 | 5.758356667 | 5.792813158 | 5.792060294 | 5.86055 | | | | |
| ZEB2 | 5.58955 | 5.600247368 | 5.602375 | 5.691709091 | | | | |
| UBE2A | 7.649666667 | 7.640873684 | 7.651729412 | 7.751495455 | | | | |
| BC040758 | 6.051266667 | 6.083378947 | 6.076677941 | 6.153059091 | | | | |
| FOXE1 | 5.774953333 | 5.767334211 | 5.776966176 | 5.876681818 | | | | |
| BCKDK | 9.482256667 | 9.443044737 | 9.485333824 | 9.583913636 | | | | |
| TRIM15 | 5.583353333 | 5.593910526 | 5.620164706 | 5.684890909 | | | | |
| HOXC10 | 6.14456 | 6.149455263 | 6.131916176 | 6.246045455 | | | | |
| NFKB1 | 11.57260667 | 11.58177368 | 11.57118529 | 11.67374091 | | | | |
| OLFR228 | 5.64159 | 5.606563158 | 5.655326471 | 5.742690909 | | | | |
| CLCN1 | 5.78522 | 5.807239474 | 5.786810294 | 5.886245455 | | | | |
| 6330579M01RIK | 5.81892 | 5.839239474 | 5.814498529 | 5.919422727 | | | | |
| SFRS2IP | 9.258286667 | 9.270178947 | 9.233055882 | 9.358495455 | | | | |
| 4930543G11RIK | 5.784193333 | 5.789855263 | 5.775158824 | 5.88425 | | | | |
| 2900090M10RIK | 5.65543 | 5.685610526 | 5.694514706 | 5.755240909 | | | | |
| LOC100048331 | 5.8999 | 5.918623684 | 5.893625 | 5.999568182 | | | | |
| TFIP11 | 8.054953333 | 8.051084211 | 8.095283824 | 8.154390909 | | | | |
| TTC4 | 8.308693333 | 8.288452632 | 8.308645588 | 8.408109091 | | | | |
| CAMK1 | 6.459003333 | 6.399568421 | 6.416069118 | 6.557886364 | | | | |
| 2600005J22RIK | 5.692316667 | 5.698397368 | 5.732252941 | 5.790959091 | | | | |
| PSCD2 | 6.155033333 | 6.126615789 | 6.134001471 | 6.253527273 | | | | |
| control_ILMN_1378911 | 5.681283333 | 5.703731579 | 5.700030882 | 5.779386364 | | | | |
| F830116E18RIK | 5.922473333 | 5.896713158 | 5.892973529 | 6.020572727 | | | | |
| ATPBD3 | 6.565073333 | 6.575857895 | 6.569377941 | 6.663122727 | | | | |
| SLC22A21 | 5.769 | 5.808589474 | 5.774551471 | 5.867009091 | | | | |
| FBXL15 | 7.304583333 | 7.278686842 | 7.283870588 | 7.402495455 | | | | |
| 4930481A15RIK | 5.86779 | 5.882173684 | 5.878910294 | 5.965627273 | | | | |
| E030016N13RIK | 6.23018 | 6.217034211 | 6.198725 | 6.328004545 | | | | |
| PDGFB | 6.229823333 | 6.231968421 | 6.243358824 | 6.327645455 | | | | |
| 4933437N03RIK | 6.072506667 | 6.089789474 | 6.098586765 | 6.169759091 | | | | |
| 2310046A06RIK | 5.654603333 | 5.617594737 | 5.633589706 | 5.7515 | | | | |
| MAK10 | 9.515693333 | 9.500657895 | 9.515651471 | 9.612581818 | | | | |
| LOC277236 | 5.9668 | 5.976176316 | 5.976830882 | 6.063481818 | | | | |
| TMEM43 | 9.091776667 | 9.138781579 | 9.132544118 | 9.1884 | | | | |
| CAMK1 | 5.496296667 | 5.521871053 | 5.513919118 | 5.592581818 | | | | |
| SPIN2 | 6.355673333 | 6.370976316 | 6.355486765 | 6.451927273 | | | | |
| V1RC23 | 5.68207 | 5.686805263 | 5.691661765 | 5.777972727 | | | | |
| ZFP239 | 7.216716667 | 7.201723684 | 7.227942647 | 7.312536364 | | | | |
| COQ6 | 9.932686667 | 9.977407895 | 9.965157353 | 10.02839545 | | | | |
| CLCNKB | 5.946433333 | 5.926278947 | 5.955783824 | 6.042031818 | | | | |
| 2310051M13RIK | 5.616203333 | 5.617539474 | 5.608520588 | 5.7118 | | | | |
| RHOT1 | 9.741433333 | 9.783013158 | 9.776666176 | 9.836890909 | | | | |
| GPR106, LGR8, GPCR | 5.783516667 | 5.794937368 | 5.813719118 | 5.878945455 | | | | |
| OLFR907 | 5.608323333 | 5.601023684 | 5.643789706 | 5.703095455 | | | | |
| TRABD | 8.528336667 | 8.528678947 | 8.508880882 | 8.623013636 | | | | |
| BLCAP | 8.161383333 | 8.124126316 | 8.163920588 | 8.255986364 | | | | |
| GM628 | 5.719166667 | 5.740194737 | 5.746419118 | 5.813490909 | | | | |
| DNM3 | 5.678993333 | 5.708965789 | 5.710380882 | 5.771918182 | | | | |
| MCPT6 | 5.812683333 | 5.779026316 | 5.782316176 | 5.905395455 | | | | |
| MAPKAPK2 | 10.01880333 | 9.9891 | 10.01956471 | 10.11131818 | | | | |
| FAM123A | 5.86123 | 5.882305263 | 5.861704412 | 5.953740909 | | | | |
| SAMD10 | 6.993723333 | 6.932505263 | 6.984454412 | 7.085995455 | | | | |
| ZFP748 | 5.80429 | 5.806384211 | 5.815305882 | 5.896513636 | | | | |
| MLANA | 5.76753 | 5.760731579 | 5.768636765 | 5.8595 | | | | |
| ITGA2B | 6.458176667 | 6.438010526 | 6.450829412 | 6.550131818 | | | | |
| CYGB | 6.101066567 | 6.091292105 | 6.119488235 | 6.193004545 | | | | |
| ATP13A1 | 8.999603333 | 9.019186842 | 9.017792647 | 9.091486364 | | | | |
| TRIM2 | 5.767873333 | 5.761813158 | 5.777947059 | 5.859163636 | | | | |
| A230084A06RIK | 5.812173333 | 5.788897368 | 5.803954412 | 5.903431818 | | | | |
| CXCL14 | 5.63365 | 5.604478947 | 5.621769118 | 5.724809091 | | | | |
| TTC15 | 7.225596667 | 7.227939474 | 7.191466176 | 7.316681818 | | | | |
| TG737RPW | 6.282606667 | 6.273515789 | 6.300575 | 6.372831818 | | | | |
| AMBRA1 | 5.725223333 | 5.745826316 | 5.748851471 | 5.815154545 | | | | |
| control_ILMN_1378677 | 5.97326 | 5.972618421 | 5.993672059 | 6.063127273 | | | | |
| PEF1 | 9.281666667 | 9.310060526 | 9.306907353 | 9.371368182 | | | | |
| D0HXS9928E | 9.81716 | 9.816639474 | 9.848302941 | 9.90685 | | | | |
| 9/2/2013 | 5.95884 | 5.979671053 | 5.968436765 | 5.048472727 | | | | |
| OLFR519 | 5.661136667 | 5.643110526 | 5.669105882 | 5.750636364 | | | | |
| OLFR312 | 5.722203333 | 5.691555263 | 5.725964706 | 5.811631818 | | | | |
| AMBRA1 | 8.782346667 | 8.732421053 | 8.754764706 | 8.87175 | | | | |
| COL4A1 | 6.219506667 | 6.194963158 | 6.209345588 | 6.308686364 | | | | |
| AI844366 | 5.80145 | 5.794457895 | 5.793163235 | 5.890395455 | | | | |
| CAR6 | 5.65408 | 5.63995 | 5.680355882 | 5.743022727 | | | | |
| PLD1 | 5.90579 | 5.893855263 | 5.927305882 | 5.994177273 | | | | |
| C230092H20RIK | 5.8978 | 5.885568421 | 5.901997059 | 5.986186364 | | | | |
| GSTK1 | 8.39841 | 8.369881579 | 8.386477941 | 8.48635 | | | | |
| EPM2A | 5.76621 | 5.761413158 | 5.760173529 | 5.853804545 | | | | |
| LOC383439 | 5.70715 | 5.681865789 | 5.707725 | 5.794672727 | | | | |
| C730048C13RIK | 5.911796667 | 5.940139474 | 5.940685294 | 5.9992 | | | | |
| 2210010L05RIK | 5.589083333 | 5.596602632 | 5.598317647 | 5.676368182 | | | | |
| NAPA | 8.002653333 | 7.993915789 | 7.994260294 | 8.089840909 | | | | |
| GABRG1 | 5.539946667 | 5.546115789 | 5.549933824 | 5.626995455 | | | | |
| 4930471I01RIK | 5.840526667 | 5.865676316 | 5.854005882 | 5.927468182 | | | | |
| COBLL1 | 7.115376667 | 7.116502632 | 7.080435294 | 7.202304545 | | | | |
| CNTLN | 5.61508 | 5.641421053 | 5.647154412 | 5.701913636 | | | | |
| TSNAXIP1 | 5.814843333 | 5.814523684 | 5.811405882 | 5.901509091 | | | | |
| A4GALT | 5.785286667 | 5.792531579 | 5.801111765 | 5.871859091 | | | | |
| CHGA | 5.746556667 | 5.719539474 | 5.755676471 | 5.832945455 | | | | |
| SOX7 | 6.187313333 | 6.202473684 | 6.163425 | 6.273668182 | | | | |
| KIF17 | 5.8875 | 5.912607895 | 5.897030882 | 5.973745455 | | | | |
| CDCA1 | 5.64207 | 5.638185474 | 5.662566176 | 5.727986364 | | | | |
| TCF7L2 | 5.724723333 | 5.710573684 | 5.716716176 | 5.810431818 | | | | |
| OLFR506 | 5.67991 | 5.537868421 | 5.635769118 | 5.765595455 | | | | |
| WNK2 | 5.703856667 | 5.706834211 | 5.718935294 | 5.799340909 | | | | |
| 8030455F02RIK | 5.73374 | 5.748163158 | 5.765951471 | 5.819077273 | | | | |
| D430013G08RIK | 5.650593333 | 5.670342105 | 5.657922059 | 5.735872727 | | | | |
| NRXN1 | 5.979573333 | 5.959471053 | 5.957466176 | 6.064813636 | | | | |
| WIZ | 7.21713 | 7.182071053 | 7.206314706 | 7.301809091 | | | | |
| BPHL | 5.726646667 | 5.735092105 | 5.714341176 | 5.811322727 | | | | |
| EG665903 | 5.54671 | 5.541965789 | 5.546788235 | 5.631263636 | | | | |
| GPSN2 | 11.36477667 | 11.34527632 | 11.37852794 | 11.44931364 | | | | |
| V1RG5 | 5.585696667 | 5.592194737 | 5.586235294 | 5.669590909 | | | | |
| LOC668779 | 5.82907 | 5.841584211 | 5.822419118 | 5.91275 | | | | |
| AW538212 | 5.662 | 5.618852632 | 5.619076471 | 5.745609091 | | | | |
| D930029H24RIK | 5.586836667 | 5.5675 | 5.567926471 | 5.670409091 | | | | |
| control_ILMN_1379309 | 5.740433333 | 5.749018421 | 5.755252941 | 5.823736364 | | | | |
| MFSD7C | 5.92774 | 5.924468421 | 5.955697059 | 6.011031818 | | | | |
| EFNA5 | 5.71599 | 5.688497368 | 5.732614706 | 5.799154545 | | | | |
| DMXL1 | 5.96065 | 5.975723684 | 5.963417647 | 6.043718182 | | | | |
| LOC276803 | 5.940693333 | 5.897260526 | 5.924936765 | 6.02375 | | | | |
| 2810043G13RIK | 5.881486667 | 5.887989474 | 5.907816176 | 5.964436364 | | | | |
| ABCF3 | 9.548456667 | 9.504076316 | 9.536877941 | 9.6311 | | | | |
| 9830166F08RIK | 5.70739 | 5.718118421 | 5.714625 | 5.789945455 | | | | |
| PDE4B | 5.75419 | 5.751107895 | 5.761052941 | 5.836522727 | | | | |
| GM378 | 5.619333333 | 5.633768421 | 5.638488235 | 5.701636364 | | | | |
| B230378H13RIK | 5.648293333 | 5.651236842 | 5.628592647 | 5.730445455 | | | | |
| 9530031H08RIK | 5.732286667 | 5.718855263 | 5.747191176 | 5.814431818 | | | | |
| 4930433I11RIK | 5.615196667 | 5.622955263 | 5.604207353 | 5.697304545 | | | | |
| CEBPE | 6.23217 | 6.227065789 | 6.229376471 | 6.314113636 | | | | |
| MTA2 | 9.862493333 | 9.858439474 | 9.847454412 | 9.943504545 | | | | |
| APOC1 | 5.5881 | 5.564128947 | 5.598686765 | 5.669086364 | | | | |
| NLRP5 | 5.79998 | 5.772057895 | 5.790597059 | 5.860922727 | | | | |
| CYP2D26 | 5.73149 | 5.724460526 | 5.741060294 | 5.812322727 | | | | |
| control_ILMN_1378609 | 5.733256667 | 5.732771053 | 5.742875 | 5.814027273 | | | | |
| OLFR524 | 5.86197 | 5.829492105 | 5.858877941 | 5.942722727 | | | | |
| LOC384689 | 5.639723333 | 5.6252 | 5.637626471 | 5.72035 | | | | |
| ADAT1 | 6.339843333 | 6.349155263 | 6.332338235 | 6.420339354 | | | | |
| CCDC15 | 5.833546667 | 5.819647368 | 5.840797059 | 5.913731818 | | | | |
| 3/8/2013 | 5.87245 | 5.886128947 | 5.855538235 | 5.952545455 | | | | |
| C230094L10RIK | 5.776726667 | 5.778110526 | 5.777417647 | 5.856627273 | | | | |
| E130113E03RIK | 5.561873333 | 5.574134211 | 5.578641176 | 5.641704545 | | | | |
| C730029N23RIK | 5.84567 | 5.8332 | 5.851813235 | 5.925118182 | | | | |
| 3/6/2013 | 7.65466 | 7.654852632 | 7.630813235 | 7.734104545 | | | | |
| LOC384155 | 5.67926 | 5.681310526 | 5.683101471 | 5.758663636 | | | | |
| MTMR6 | 5.577 | 5.549068421 | 5.543748529 | 5.656304545 | | | | |
| 9790357_3511_RC | 5.089263333 | 5.670352632 | 5.704086765 | 5.768504545 | | | | |
| OLFR11 | 5.660956667 | 5.678615789 | 5.662433824 | 5.740054545 | | | | |
| 2510025K24RIK | 5.582343333 | 5.614260526 | 5.610119118 | 5.661422727 | | | | |
| SLC7A14 | 5.839583333 | 5.813355263 | 5.838160294 | 5.918435364 | | | | |
| 5830448L21RIK | 5.74232 | 5.713471053 | 5.708772059 | 5.821154545 | | | | |
| 4631434O19RIK | 6.00784 | 6.001673684 | 6.026148529 | 6.086531818 | | | | |
| C230011P08RIK | 5.752406667 | 5.742231579 | 5.757427941 | 5.831013636 | | | | |
| OLFR483 | 5.625586667 | 5.602889474 | 5.632177941 | 5.704077273 | | | | |
| LOC382853 | 5.963353333 | 5.971465789 | 5.954307353 | 6.041727273 | | | | |
| SFTD6 | 8.83772 | 8.819034211 | 8.829725 | 8.915090909 | | | | |
| CBFA2T2 | 6.07727 | 6.059334211 | 6.080733824 | 6.1556 | | | | |
| LOC100045448 | 5.673983333 | 5.660994737 | 5.677317647 | 5.752286364 | | | | |
| OLFR535 | 5.571573333 | 5.596031579 | 5.600444118 | 5.649868182 | | | | |
| ATP2C1 | 6.559026667 | 6.536521053 | 6.572148529 | 6.637072727 | | | | |
| KLHDC8B | 5.822313313 | 5.821168421 | 5.831194118 | 5.899663636 | | | | |
| ADAMDEC1 | 5.72808 | 5.726289474 | 5.725811765 | 5.80525 | | | | |
| UNK | 8.188016667 | 8.146186842 | 8.196697059 | 8.2651 | | | | |
| 5730460C07RIK | 6.136043333 | 6.140568421 | 6.114717647 | 6.213077273 | | | | |
| KLHL26 | 9.295173333 | 9.293997368 | 9.285769118 | 9.371863636 | | | | |
| LIG3 | 8.005243333 | 7.995089474 | 7.992510294 | 8.081459091 | | | | |
| PAM | 5.65448 | 5.659834211 | 5.671320588 | 5.730622727 | | | | |
| V2R15 | 5.637346667 | 5.614844737 | 5.596172059 | 5.713459091 | | | | |
| 6430605CD3RIK | 5.550003333 | 5.554918421 | 5.54665 | 5.625927273 | | | | |
| 8430429KD9RIK | 6.45922 | 6.441084211 | 6.474969118 | 6.535031818 | | | | |
| C430004E15RIK | 8.70302 | 8.674578947 | 8.702492647 | 8.778827273 | | | | |
| LOC244335 | 5.711056667 | 5.718365789 | 5.732983824 | 5.786463636 | | | | |
| DOXL2 | 5.788483333 | 5.74895 | 5.735679412 | 5.863831818 | | | | |
| 4930554K12RIK | 5.8731 | 5.877186842 | 5.883825 | 5.948418182 | | | | |
| TGFBR2 | 6.004693333 | 5.996826316 | 5.984408824 | 6.079877273 | | | | |
| HPX | 5.89602 | 5.880847368 | 5.874075 | 5.971186364 | | | | |
| 2900052P03RIK | 6.166873333 | 6.185847368 | 6.173483824 | 6.241986364 | | | | |
| CPXCR1 | 5.67251 | 5.677502632 | 5.671010294 | 5.747422727 | | | | |
| TAS2R106 | 5.694626667 | 5.691915789 | 5.704780882 | 5.769118182 | | | | |
| 4632404B13RIK | 5.94389 | 5.950968421 | 5.949844118 | 6.018218182 | | | | |
| 1600012K10RIK | 5.759546667 | 5.761076316 | 5.748283824 | 5.833790909 | | | | |
| OLFR121 | 5.667883333 | 5.671623684 | 5.673066176 | 5.741809091 | | | | |
| 6130401J04RIK | 5.64243 | 5.654536842 | 5.653991176 | 5.716331818 | | | | |
| CLPTM1L | 11.29636667 | 11.30115 | 11.30577794 | 11.36985 | | | | |
| 4732490P12 | 5.554813333 | 5.5452 | 5.569813235 | 5.628077273 | | | | |
| LOC545289 | 6.18028 | 6.178142105 | 6.194416176 | 6.253086364 | | | | |
| ATP6V0D1 | 10.56093333 | 10.54983158 | 10.56906471 | 10.63357727 | | | | |
| EG433144 | 5.7232 | 5.727760526 | 5.728964706 | 5.795736364 | | | | |
| RAB11FIP5 | 6.24462 | 6.244602632 | 6.211269118 | 6.316795455 | | | | |
| OLFR94 | 5.866656667 | 5.867705263 | 5.842164706 | 5.938768182 | | | | |
| LOC245453 | 5.83364 | 5.832957895 | 5.845691176 | 5.905668182 | | | | |
| V1RD7 | 5.525103333 | 5.525413158 | 5.519311765 | 5.597113636 | | | | |
| ANKRD35 | 5.805983333 | 5.794584211 | 5.766820588 | 5.877945455 | | | | |
| SETD6 | 8.39426 | 8.365178947 | 8.388533824 | 8.466163636 | | | | |
| SQLE | 5.744813333 | 5.742528947 | 5.748705882 | 5.816636364 | | | | |
| A330004N04RIK | 5.65038 | 5.638315789 | 5.630204412 | 5.722040909 | | | | |
| USP22 | 8.500313333 | 8.479518421 | 8.469433824 | 8.571968182 | | | | |
| control_ILMN_1378808 | 5.613206667 | 5.619639474 | 5.619225 | 5.684513636 | | | | |
| AK122498 | 5.750016667 | 5.754710526 | 5.748085294 | 5.821009091 | | | | |
| LOC381546 | 5.912206667 | 5.904515789 | 5.922085294 | 5.983263636 | | | | |
| ATP7B | 5.68384 | 5.670181579 | 5.663311765 | 5.754631818 | | | | |
| RBM41 | 5.543303333 | 5.5455 | 5.531645588 | 5.613786364 | | | | |
| D610007L01RIK | 5.754013333 | 5.758968421 | 5.733432353 | 5.82365 | | | | |
| 2610528H13RIK | 5.603936667 | 5.579705263 | 5.602720588 | 5.673463636 | | | | |
| D030018H12RIK | 5.641683333 | 5.647418421 | 5.648041176 | 5.711190909 | | | | |
| CDH12 | 5.66794 | 5.682739474 | 5.660370588 | 5.737386364 | | | | |
| E430014N10RIK | 5.662576667 | 5.636118421 | 5.650170588 | 5.731740909 | | | | |
| LRRC17 | 5.906216667 | 5.904839474 | 5.911247059 | 5.975331818 | | | | |
| 4931417G12RIK | 5.614283333 | 5.617 | 5.594630882 | 5.683309091 | | | | |
| OLFR615 | 5.721326667 | 5.703034211 | 5.727417647 | 5.790331818 | | | | |
| LOC631010 | 5.73648 | 5.73475 | 5.710307353 | 5.805286364 | | | | |
| PPP1CA | 12.43433667 | 12.39944474 | 12.42297941 | 12.50304545 | | | | |
| E130009J12RIK | 5.743916667 | 5.723594737 | 5.736714706 | 5.812536364 | | | | |
| CD209A | 5.65628 | 5.624876316 | 5.639332353 | 5.724654545 | | | | |
| TFPI | 5.627863333 | 5.604692105 | 5.625113235 | 5.696140909 | | | | |
| 5730530J16RIK | 5.65864 | 5.672378947 | 5.668261765 | 5.726768182 | | | | |
| EG666609 | 13.11461333 | 13.13349737 | 13.13128971 | 13.18201818 | | | | |
| VPS13D | 5.825393333 | 5.825310521 | 5.823529412 | 5.892627273 | | | | |
| BCL11A | 5.64446 | 5.628544737 | 5.630797059 | 5.711472727 | | | | |
| PVRL2 | 5.80492 | 5.796597368 | 5.821182353 | 5.87185 | | | | |
| GM392 | 5.49587 | 5.480410526 | 5.501230882 | 5.562286364 | | | | |
| 3110012M05RIK | 5.661996667 | 5.661102632 | 5.650423525 | 5.728168182 | | | | |
| 6330437I11RIK | 5.88513 | 5.868713158 | 5.863848529 | 5.950863636 | | | | |
| 4921537F17RIK | 5.829483333 | 5.817065789 | 5.833544118 | 5.894563636 | | | | |
| MAK10 | 8.8104 | 8.765121053 | 8.803444118 | 8.874963636 | | | | |
| FTS | 5.683446667 | 5.655231579 | 5.667736765 | 5.747927273 | | | | |
| PHXR5 | 5.872406067 | 5.83705 | 5.835738235 | 5.936231818 | | | | |
| genericLS | 6.13706 | 6.099334211 | 6.107116176 | 6.200122727 | | | | |
| DEFCR-R51 | 5.621336667 | 5.599907895 | 5.619592647 | 5.683168182 | | | | |
| TSSK6 | 5.885473333 | 5.876747368 | 5.882098529 | 5.946995455 | | | | |
| LOC100044218 | 5.70159 | 5.688944737 | 5.699707353 | 5.762777273 | | | | |
| GULO | 5.675643333 | 5.633252632 | 5.673354412 | 5.736640909 | | | | |
| LOC381636 | 5.679483333 | 5.660081579 | 5.653820588 | 5.740377273 | | | | |
| 1810019D21RIK | 5.926816667 | 5.899373684 | 5.896448529 | 5.987313636 | | | | |
| LOC100039649 | 5.650293333 | 5.647105263 | 5.650401471 | 5.710781818 | | | | |
| 2900097C17RIK | 5.539346667 | 5.523955263 | 5.519045588 | 5.599572727 | | | | |
| SEC11A | 11.49351 | 11.48228158 | 11.46473971 | 11.55365909 | | | | |
| BOC | 6.039113333 | 6.034384211 | 6.049230882 | 6.099004545 | | | | |
| EG224576 | 5.743756667 | 5.712397368 | 5.723908824 | 5.802577273 | | | | |
| E030045A12RIK | 5.811073333 | 5.802289474 | 5.779011765 | 5.86955 | | | | |
| TCF7L2 | 5.820176667 | 5.799102632 | 5.790647059 | 5.877909091 | | | | |
| 3732412D22RIK | 5.46195 | 5.456131579 | 5.453826471 | 5.518868182 | | | | |
| SCL0002508.1_73 | 5.615476667 | 5.575942105 | 5.598632353 | 5.671372727 | | | | |
| D030002E05RIK | 5.522803333 | 5.523189474 | 5.500327941 | 5.577595455 | | | | |

| | | | | | | | |
|---|---|---|---|---|---|---|---|
| | | Medium vs. | | | | | |
| | | Reference | | | Reference | Reference | |
| | | Standard | Medium vs. | Medium vs | Standard vs | Standard vs | GA vs. |
| Gene | ID | pval | GA pval | generic pval | GA pval | generic pval | generic pval |

DOWN IN generic ONLY

| | | | | | | | |
|---|---|---|---|---|---|---|---|
| SATB1 | ILMN_1240428 | 0.07911534 | 0.37144914 | 1.8348E−08 | 0.32081734 | 1.2391E−06 | 3.3523E−08 |
| ANKRD37 | ILMN_2595857 | 0.85603343 | 0.11987312 | 5.0602E−05 | 0.1157346 | 2.4144E−06 | 1.2346E−08 |
| PDX1 | ILMN_1229318 | 0.05253197 | 0.2345219 | 3.0177E−07 | 0.32074143 | 7.3944E−06 | 5.6956E−08 |
| A830055I09RIK | ILMN_1252200 | 0.11618297 | 0.49076503 | 1.6533E−05 | 0.27282917 | 0.00185251 | 3.3438E−05 |
| CD2 | ILMN_2753697 | 0.27081402 | 0.2043282 | 1.1319E−07 | 0.98324628 | 7.8538E−08 | 1.0361E−10 |
| IFIT3 | ILMN_2944666 | 0.4738633 | 0.81801934 | 4.5724E−05 | 0.18790987 | 1.54E−09 | 2.8447E−08 |
| TMSB10 | ILMN_3145331 | 0.21029649 | 0.97008239 | 5.9274E−07 | 0.11049461 | 4.1918E−06 | 1.5861E−10 |
| PELI1 | ILMN_1239770 | 0.86490877 | 0.09870079 | 7.6688E−08 | 0.10384766 | 7.9696E−09 | 6.2297E−08 |
| LOC665425 | ILMN_1213664 | 0.23297648 | 0.25120955 | 3.1475E−05 | 0.78597257 | 0.00061167 | 2.1866E−05 |
| 5830468F06RIK | ILMN_1247281 | 0.3090019 | 0.17260429 | 6.0433E−07 | 0.87028716 | 1.9117E−05 | 7.5927E−07 |
| BCL11B | ILMN_2611022 | 0.61541666 | 0.16410668 | 0.00078873 | 0.34999007 | 4.7031E−05 | 1.3983E−07 |
| SAMHD1 | ILMN_1232707 | 0.05814465 | 0.1358425 | 0.00017313 | 0.21061544 | 0.00020723 | 2.7253E−07 |
| IGFBP4 | ILMN_1258988 | 0.82474932 | 0.2971467 | 0.0024654 | 0.31582363 | 0.00028207 | 0.00175534 |
| OTTMUSG00000007855 | ILMN_3123441 | 0.11070867 | 0.08547026 | 0.00200816 | 0.9832071 | 0.04667215 | 0.04859397 |
| A530024C08RIK | ILMN_1243902 | 0.41733897 | 0.23521863 | 5.8513E−05 | 0.77449484 | 0.00148812 | 0.00088129 |
| D330001F19RIK | ILMN_1259917 | 0.54143382 | 0.24928554 | 2.5408E−05 | 0.59607246 | 0.00011785 | 0.00034508 |
| C230075M21RIK | ILMN_1215649 | 0.20048573 | 0.66752912 | 0.00200298 | 0.36009335 | 8.7673E−06 | 0.00041343 |
| TERF2IP | ILMN_2986458 | 0.17591671 | 0.1139061 | 0.00015309 | 0.91082142 | 0.00299315 | 0.00025266 |
| ARID5B | ILMN_3031781 | 0.48039514 | 0.63560294 | 0.00015248 | 0.19905299 | 0.00015866 | 0.00037839 |
| VPS13A | ILMN_2561533 | 0.17374866 | 0.05433989 | 8.7726E−05 | 0.63593438 | 0.00069405 | 0.00131973 |
| PPM1B | ILMN_2729953 | 0.21539059 | 0.40196624 | 0.0040908 | 0.57702572 | 0.01403207 | 0.00503541 |
| ZFP281 | ILMN_1220893 | 0.14622272 | 0.17820672 | 7.192E−06 | 0.6818773 | 3.0398E−05 | 8.1174E−07 |
| 2610027C15RIK | ILMN_2890357 | 0.17393869 | 0.10511917 | 0.00018625 | 0.8704277 | 0.0003711 | 0.00040132 |
| BC031575 | ILMN_2703242 | 0.05104096 | 0.0844921 | 0.00065581 | 0.72788898 | 0.01360668 | 0.00718183 |
| PELI1 | ILMN_2627441 | 0.69993334 | 0.62839012 | 0.00014024 | 0.94889436 | 4.6366E−06 | 1.1293E−06 |
| RNF125 | ILMN_2917338 | 0.71751754 | 0.21101594 | 0.00081812 | 0.30322086 | 0.00017849 | 0.00339438 |
| 1500005K14RIK | ILMN_1217606 | 0.10066065 | 0.26627278 | 0.00078516 | 0.30239173 | 0.01326166 | 0.00024226 |
| 1110059E24RIK | ILMN_2846731 | 0.0550847 | 0.29416851 | 4.4207E−06 | 0.15740767 | 0.00036598 | 1.4474E−07 |
| GSN | ILMN_2605645 | 0.11878006 | 0.10970834 | 0.00341431 | 0.71650151 | 0.0173612 | 0.00330019 |
| ARL6IP5 | ILMN_1241924 | 0.1994807 | 0.50995839 | 0.00036067 | 0.39437768 | 0.00142205 | 0.00010627 |
| NOTCH1 | ILMN_1241915 | 0.18364872 | 0.94760135 | 0.00072539 | 0.07628277 | 1.4502E−06 | 3.3341E−06 |
| 9130422G05RIK | ILMN_2633062 | 0.07359432 | 0.05531104 | 0.00010494 | 0.83711906 | 0.02159426 | 0.00415836 |
| LOC333818 | ILMN_1244672 | 0.14738668 | 0.17882651 | 0.0016716 | 0.83534848 | 0.01963961 | 0.00965869 |
| PTPRC | ILMN_1212836 | 0.60662184 | 0.2797467 | 0.00865581 | 0.51601654 | 0.00367386 | 0.02465436 |
| KMO | ILMN_1224155 | 0.19403001 | 0.13773846 | 0.00016815 | 0.75603238 | 0.00118003 | 0.00579415 |
| WIPF1 | ILMN_1257209 | 0.51865115 | 0.38594682 | 0.00038569 | 0.84119617 | 1.8977E−05 | 2.1965E−05 |
| FOXK1 | ILMN_3059798 | 0.22146868 | 0.35931982 | 8.7596E−05 | 0.69209323 | 0.00128851 | 0.00028068 |
| 2310032M22RIK | ILMN_2550291 | 0.59008093 | 0.37289744 | 0.00020033 | 0.1046556 | 1.0638E−05 | 0.00012106 |
| RPS25 | ILMN_2887619 | 0.34627656 | 0.77685937 | 0.03034502 | 0.15501605 | 0.00438895 | 0.03226412 |
| LOC665446 | ILMN_1257496 | 0.19329193 | 0.11013072 | 0.00503496 | 0.81016026 | 1.097E−05 | 1.8517E−06 |
| LOC237512 | ILMN_2533952 | 0.10185041 | 0.06643866 | 0.00038583 | 0.99416409 | 0.00426908 | 0.00145306 |
| LOC386068 | ILMN_1214874 | 0.08141757 | 0.46236733 | 0.00027129 | 0.14381003 | 0.02213701 | 0.0001023 |
| ZFP429 | ILMN_1254717 | 0.07332322 | 0.14675334 | 0.00015711 | 0.57011156 | 0.01213883 | 0.00151289 |
| BCL2L11 | ILMN_2590638 | 0.30446502 | 0.40049526 | 0.00017608 | 0.6930002 | 0.00060191 | 4.8156E−05 |
| CUGBP2 | ILMN_2594575 | 0.87267521 | 0.1578596 | 0.00606795 | 0.1445108 | 0.00117238 | 0.02174746 |
| SLPN1 | ILMN_2663930 | 0.39293473 | 0.51218967 | 0.00170587 | 0.70949257 | 4.9201E−05 | 8.3548E−06 |
| KLHDC1 | ILMN_2649635 | 0.84189965 | 0.07760558 | 0.00155996 | 0.08479279 | 0.00098869 | 0.03185451 |
| CRAMP1L | ILMN_1247318 | 0.20143537 | 0.23644921 | 0.00101289 | 0.95893024 | 0.00078111 | 0.00112658 |
| CTSE | ILMN_1228235 | 0.99911127 | 0.41065532 | 0.00070458 | 0.36116375 | 0.0002081 | 0.00018402 |
| CGGBP1 | ILMN_2668387 | 0.57946818 | 0.88874843 | 8.6419E−05 | 0.54605077 | 2.2894E−05 | 1.6764E−07 |
| A130075K23RIK | ILMN_1217850 | 0.81537177 | 0.32245027 | 0.00051066 | 0.16564844 | 3.4193E−05 | 0.00308157 |
| CDKN1B | ILMN_2923960 | 0.12950768 | 0.13195636 | 2.6887E−06 | 0.89753459 | 0.00042167 | 0.00010697 |
| HLTF | ILMN_2604086 | 0.10435469 | 0.0745037 | 0.00087569 | 0.96981342 | 0.01569298 | 0.01190867 |
| FNBP4 | ILMN_1260410 | 0.83081352 | 0.24441759 | 0.00263707 | 0.30109811 | 0.00158996 | 0.00944233 |
| LOC623121 | ILMN_2526938 | 0.11929339 | 0.36859146 | 0.0126513 | 0.32751281 | 6.5544E−05 | 0.0001654 |
| 3300001P08RIK | ILMN_2798797 | 0.26036986 | 0.09061791 | 0.00118783 | 0.72277312 | 0.00256883 | 0.0005881 |
| LOC100048622 | ILMN_2677056 | 0.8875675 | 0.61503938 | 0.00618713 | 0.43247407 | 0.00034098 | 0.00260119 |
| C430005L07RIK | ILMN_1231989 | 0.05812598 | 0.06291277 | 0.00060981 | 0.8300766 | 0.01375588 | 0.00724939 |
| ZFP654 | ILMN_2634157 | 0.06904386 | 0.45768661 | 7.3775E−06 | 0.21584233 | 0.00045141 | 1.8816E−05 |
| E030040J04RIK | ILMN_2582659 | 0.6078898 | 0.94589189 | 0.0006524 | 0.59757263 | 1.5494E−05 | 5.6983E−05 |
| ARL4C | ILMN_1232537 | 0.74119045 | 0.12627286 | 0.00222578 | 0.05116815 | 0.00522971 | 3.7398E−06 |
| ARID4A | ILMN_1249368 | 0.63477844 | 0.22538884 | 0.01297411 | 0.45126135 | 0.01447908 | 0.02906182 |
| LOC546629 | ILMN_2514130 | 0.1422128 | 0.11491335 | 0.00013948 | 0.84250699 | 0.00317588 | 0.00678748 |
| TDP1 | ILMN_1222101 | 0.20471786 | 0.21603931 | 0.00042705 | 0.76601877 | 0.00953396 | 0.00159959 |
| CUL1 | ILMN_1226323 | 0.09343825 | 0.06890272 | 0.0020377 | 0.761411325 | 0.04176493 | 0.00665118 |
| D030041G07RIK | ILMN_1242969 | 0.18864709 | 0.06254915 | 0.00099329 | 0.61432768 | 0.00800136 | 0.01658998 |
| E130014H08RIK | ILMN_2680388 | 0.06694365 | 0.06110366 | 6.3688E−05 | 0.9737956 | 0.01184346 | 0.01143026 |
| TRAF5 | ILMN_2440604 | 0.52455627 | 0.24011296 | 0.00690509 | 0.52721633 | 0.00289499 | 0.02142899 |
| PPP2R5C | ILMN_2742461 | 0.11379206 | 0.08222043 | 0.00037899 | 0.92947841 | 0.01165728 | 0.00335504 |
| MTF2 | ILMN_2703913 | 0.20036311 | 0.29858647 | 0.00375869 | 0.69841001 | 0.03389861 | 0.01319182 |
| WDR20B | ILMN_1235032 | 0.23781394 | 0.47851722 | 0.00019242 | 0.45624566 | 6.383E−05 | 6.7883E−06 |
| RNF125 | ILMN_1232600 | 0.9821989 | 0.0966035 | 0.00041169 | 0.0959517 | 0.00113953 | 5.261E−07 |
| 9330168G06RIK | ILMN_1246293 | 0.54190202 | 0.48310219 | 0.00024149 | 0.94557713 | 0.00139303 | 0.00147706 |
| LAMP2 | ILMN_2577206 | 0.81917654 | 0.05394462 | 0.00083459 | 0.07901609 | 0.00135313 | 0.01849123 |
| JMJD1A | ILMN_3043245 | 0.31845749 | 0.65249285 | 0.00020808 | 0.07212806 | 0.00038958 | 5.2342E−07 |
| ST8SIA4 | ILMN_2784230 | 0.13498007 | 0.47574935 | 0.00103374 | 0.26063235 | 0.02824889 | 0.00078328 |
| LRBA | ILMN_2659960 | 0.17994841 | 0.13393498 | 0.00174452 | 0.93977444 | 0.00204093 | 0.00098864 |
| TDE2 | ILMN_2775381 | 0.95693713 | 0.13126573 | 0.00315917 | 0.09028776 | 0.00042239 | 0.02374199 |
| 9/6/2013 | ILMN_2681293 | 0.63762827 | 0.24964088 | 0.01397803 | 0.4764143 | 0.0147676 | 0.04238798 |
| D830015B12RIK | ILMN_1234367 | 0.30565462 | 0.07227944 | 0.00022702 | 0.52306092 | 0.00800972 | 0.0113831 |
| PARD6G | ILMN_1237631 | 0.67588419 | 0.678385 | 0.00086841 | 0.96479588 | 0.00052187 | 0.00012651 |
| 4933400A11RIK | ILMN_1250324 | 0.19354888 | 0.49469851 | 0.0004042 | 0.34066981 | 0.01872749 | 0.00029194 |
| 2210016L21RIK | ILMN_1251123 | 0.06841243 | 0.16045005 | 0.00035392 | 0.64566996 | 0.00449141 | 0.00251111 |
| MDM1 | ILMN_2754667 | 0.25199431 | 0.24519798 | 0.00035648 | 0.87341123 | 0.00675749 | 0.00112085 |
| CDC37L1 | ILMN_1242724 | 0.43296504 | 0.76183799 | 0.00182347 | 0.50487916 | 0.00996967 | 0.00041643 |
| B230215E14RIK | ILMN_1229011 | 0.59291696 | 0.19530417 | 0.00010561 | 0.48368188 | 0.00096959 | 0.00112415 |
| POM121 | ILMN_1237596 | 0.42663271 | 0.8235093 | 0.00231014 | 0.21790859 | 5.4061E−05 | 0.0005903 |
| FOXN3 | ILMN_2844097 | 0.26359703 | 0.20440307 | 0.00466812 | 0.89339493 | 0.01751217 | 0.00321484 |
| A130021K16RIK | ILMN_1251811 | 0.81955148 | 0.44269848 | 8.6977E−05 | 0.27630522 | 6.7923E−05 | 1.3989E−05 |
| DAG1 | ILMN_2853957 | 0.42523807 | 0.06023444 | 0.00579353 | 0.24060178 | 0.00467159 | 0.02119796 |
| BIRC6 | ILMN_1249233 | 0.45362784 | 0.33538866 | 0.00037832 | 0.83092547 | 0.00198776 | 0.00308862 |
| 4930529C04RIK | ILMN_2725583 | 0.06986766 | 0.17113468 | 0.0008444 | 0.33339434 | 0.02333411 | 0.00058033 |
| SLC16A6 | ILMN_1258950 | 0.2276815 | 0.18533844 | 0.00175895 | 0.91408667 | 0.03596058 | 0.04740725 |
| LRRC40 | ILMN_2647476 | 0.46464479 | 0.06010376 | 0.00055192 | 0.24061743 | 0.00071114 | 0.00227639 |
| BC051070 | ILMN_1231143 | 0.21162255 | 0.64642365 | 0.00131092 | 0.3151572 | 0.00760443 | 0.00044444 |
| LOC100045804 | ILMN_1234370 | 0.33824884 | 0.17904072 | 0.00022809 | 0.87467054 | 0.00400187 | 0.00073379 |
| HSH2D | ILMN_1239119 | 0.14711922 | 0.54294216 | 0.01243575 | 0.22836928 | 0.03227864 | 0.00413834 |
| VPS26B | ILMN_2631192 | 0.48773509 | 0.96099036 | 0.03419795 | 0.30653981 | 0.04809562 | 0.00115122 |
| ST6GAL2 | ILMN_2593002 | 0.95583357 | 0.65485319 | 0.00328931 | 0.63896784 | 0.00013997 | 9.6793E−06 |
| TMPRSS11A | ILMN_2890069 | 0.28670671 | 0.07813886 | 0.00838506 | 0.57793371 | 0.03312193 | 0.04134015 |
| RAB27A | ILMN_2614966 | 0.3536785 | 0.84760602 | 4.1275E−05 | 0.14171169 | 0.00031821 | 2.3052E−08 |
| LOC674087 | ILMN_1227317 | 0.74280813 | 0.5889824 | 0.03565042 | 0.85184119 | 0.04321284 | 0.04850425 |
| 1600012H06RIK | ILMN_2718861 | 0.8735272 | 0.68824217 | 0.0058169 | 0.80100365 | 0.00306695 | 0.00319401 |
| 4733401O04RIK | ILMN_1241389 | 0.06778378 | 0.39528806 | 0.00147672 | 0.23116877 | 0.04814609 | 0.00361635 |
| OLFR504 | ILMN_3161078 | 0.79013139 | 0.33133578 | 0.00059741 | 0.1655799 | 9.368E−05 | 0.00131063 |
| ADRA1B | ILMN_2606415 | 0.05675631 | 0.28864518 | 0.00073372 | 0.16483949 | 0.04210999 | 0.00042662 |
| RAB6B | ILMN_2761430 | 0.47741529 | 0.1361146 | 1.3855E−05 | 0.45580546 | 0.00087661 | 0.00243207 |
| HBB-B1 | ILMN_2545963 | 0.46626213 | 0.31620323 | 0.0349087 | 0.86663622 | 0.04746168 | 0.03081781 |
| ARHGAP12 | ILMN_1227256 | 0.48174007 | 0.34807939 | 0.00021055 | 0.85729833 | 0.00076744 | 0.0003831 |
| TYKI | ILMN_1240864 | 0.75891365 | 0.23842313 | 0.03489891 | 0.05197847 | 0.00364412 | 0.01758943 |
| LOC100048616 | ILMN_1241963 | 0.71621729 | 0.39548918 | 0.00603366 | 0.63958983 | 0.00574245 | 0.00430463 |
| 2600011C06RIK | ILMN_1220104 | 0.86806699 | 0.36310064 | 0.00494841 | 0.21110047 | 0.00042852 | 0.01324941 |
| BIRC6 | ILMN_1246660 | 0.06729015 | 0.10473405 | 0.00131141 | 0.76878822 | 0.01213217 | 0.01538146 |
| RGS19 | ILMN_2752044 | 0.33869851 | 0.05136737 | 0.00179044 | 0.46155141 | 0.03845813 | 0.04785331 |
| C130064E22RIK | ILMN_1221892 | 0.27374297 | 0.29078426 | 0.00085236 | 0.77212634 | 0.02887937 | 0.00455809 |
| ITK | ILMN_2613422 | 0.24265705 | 0.19958443 | 0.00336477 | 0.90260442 | 0.00011044 | 5.597E−05 |
| LCP2 | ILMN_1216191 | 0.92201403 | 0.37692413 | 0.00376734 | 0.28934791 | 0.00267784 | 0.01298147 |
| RBM18 | ILMN_2978585 | 0.8463194 | 0.33897524 | 0.03431259 | 0.14448539 | 0.00400462 | 0.02407365 |
| ZAP70 | ILMN_1223542 | 0.90443065 | 0.70542805 | 0.0456215 | 0.54050797 | 0.01054876 | 0.01882925 |
| control_ILMN_1378612 | ILMN_1378612 | 0.97371919 | 0.46606042 | 0.00089929 | 0.41724356 | 0.00081739 | 0.00168715 |
| MAT2A | ILMN_1258415 | 0.93943282 | 0.94206707 | 0.03519559 | 0.85852182 | 0.01473593 | 0.0087062 |
| 2210018M11RIK | ILMN_2521686 | 0.42671565 | 0.29390195 | 0.00476184 | 0.94616909 | 0.02497546 | 0.00791973 |
| ASH1L | ILMN_1245687 | 0.97479233 | 0.11885674 | 0.00538089 | 0.06492972 | 0.00097574 | 0.02416363 |
| LOXL3 | ILMN_2665272 | 0.09179491 | 0.31274257 | 0.0005144 | 0.24542621 | 0.04425972 | 0.0005322 |
| UTX | ILMN_2506528 | 0.47882605 | 0.36686461 | 0.0141063 | 0.84515672 | 0.00358262 | 0.00949066 |
| A730082L10RIK | ILMN_1220032 | 0.40329585 | 0.06661184 | 0.0020628 | 0.32206538 | 0.0062779 | 0.02207392 |
| ITGA6 | ILMN_1223670 | 0.4066823 | 0.4327231 | 0.0030597 | 0.99948811 | 0.0018074 | 0.00453858 |
| control_ILMN_1378679 | ILMN_1378679 | 0.78763578 | 0.05260707 | 0.00353159 | 0.07175941 | 0.0028401 | 0.03376404 |
| 4930449I04RIK | ILMN_1238088 | 0.20517346 | 0.23262472 | 3.0723E−05 | 0.89759905 | 0.00106926 | 0.00087064 |
| TSC1 | ILMN_2423383 | 0.67101795 | 0.22949592 | 0.00458673 | 0.40105382 | 0.00459241 | 0.01816998 |
| MSH3 | ILMN_3068452 | 0.9862047 | 0.38859095 | 0.00291834 | 0.30992825 | 0.00038421 | 0.01016092 |
| 9330171B01RIK | ILMN_1234726 | 0.05502213 | 0.46148029 | 0.00133591 | 0.12649278 | 0.04508408 | 0.001052 |
| UGT2A1 | ILMN_2434333 | 0.71050586 | 0.72182397 | 0.00431801 | 0.41667972 | 0.00069414 | 0.00299609 |
| C130078D09RIK | ILMN_1244755 | 0.12786041 | 0.32508824 | 0.00313825 | 0.47915751 | 0.00181499 | 0.0022079 |
| RHBDF2 | ILMN_1221129 | 0.84184754 | 0.5974591 | 0.02089328 | 0.79562913 | 0.04903139 | 0.01622124 |
| INPP4B | ILMN_1226239 | 0.08450582 | 0.20237327 | 0.03058425 | 0.55690542 | 8.8074E−05 | 0.00041 |
| NIPSNAP1 | ILMN_2619861 | 0.52904018 | 0.46136801 | 0.00116519 | 0.11378299 | 7.689E−05 | 0.00076877 |
| LPIN2 | ILMN_1220680 | 0.82909927 | 0.4643068 | 0.01188671 | 0.62183234 | 0.01800377 | 0.01376318 |
| 4833420G17RIK | ILMN_1242705 | 0.11058607 | 0.9985108 | 0.00140036 | 0.05589185 | 0.02982991 | 0.00016301 |
| NT5C | ILMN_1223097 | 0.96869979 | 0.15110506 | 0.00503984 | 0.0966739 | 0.0004879 | 0.04828357 |
| D730033A03RIK | ILMN_1237794 | 0.10532816 | 0.07934958 | 0.00111308 | 0.99661081 | 0.01929895 | 0.01590843 |
| 1700011H14RIK | ILMN_1240624 | 0.42648881 | 0.31978764 | 0.00264882 | 0.99210061 | 0.00666403 | 0.00118018 |
| 4921505C17RIK | ILMN_2552030 | 0.32607023 | 0.09618019 | 0.00231432 | 0.55575949 | 0.0192574 | 0.02775372 |
| ZSCAN2 | ILMN_2481127 | 0.10166694 | 0.50491577 | 0.00258406 | 0.19200936 | 0.04933246 | 0.0019485 |
| ATL1 | ILMN_2689284 | 0.0990851 | 0.09092134 | 0.00228633 | 0.97929005 | 0.04918349 | 0.04782988 |
| COPZ1 | ILMN_1223544 | 0.55643832 | 0.21087121 | 0.00791367 | 0.4514929 | 0.00949234 | 0.04313591 |
| 4921506M07RIK | ILMN_1226012 | 0.76721403 | 0.18679607 | 0.00499298 | 0.28451218 | 0.00614653 | 0.0237657 |
| 4921506J03RIK | ILMN_2541863 | 0.05819601 | 0.23888611 | 0.00023403 | 0.37181927 | 0.01993083 | 0.00337236 |
| GADD45GIP1 | ILMN_2830860 | 0.74738281 | 0.3343774 | 0.00653881 | 0.16616339 | 0.00330039 | 0.03565601 |
| RDH1 | ILMN_1230677 | 0.81344903 | 0.29787461 | 0.00081047 | 0.36612833 | 0.00024803 | 0.00234843 |
| IKZF5 | ILMN_2963412 | 0.43992194 | 0.44291872 | 0.01647085 | 0.85895934 | 0.02490897 | 0.00712778 |
| LOC385046 | ILMN_2541806 | 0.430148 | 0.14005669 | 0.00103041 | 0.47036209 | 0.00129615 | 0.00684371 |
| BC057371 | ILMN_2676745 | 0.31452008 | 0.53428622 | 0.00259007 | 0.48069658 | 0.00015576 | 1.2165E−05 |
| 9830118G07RIK | ILMN_1214091 | 0.19061978 | 0.17683894 | 0.00599888 | 0.84461581 | 0.03266526 | 0.01003646 |
| MRPS31 | ILMN_2723162 | 0.15887557 | 0.32092643 | 0.00083416 | 0.41732792 | 0.04866787 | 0.00166947 |
| 1600029O10RIK | ILMN_2546461 | 0.09970313 | 0.9814076 | 0.0169889 | 0.05149958 | 8.1725E−05 | 0.01304123 |
| OLFR125 | ILMN_2730288 | 0.1313929 | 0.58003222 | 0.00034065 | 0.29296729 | 0.03092837 | 0.00139305 |
| SPCS2 | ILMN_2762956 | 0.17612194 | 0.31317607 | 0.01153661 | 0.60798628 | 6.6576E−05 | 0.00013104 |
| BCL10 | ILMN_1215785 | 0.51502954 | 0.34312715 | 0.00677667 | 0.8020252 | 0.02353642 | 0.02396163 |
| BCL11B | ILMN_1223119 | 0.19781849 | 0.24316803 | 0.02507492 | 0.69495974 | 0.00053035 | 0.00012873 |
| control_ILMN_1379152 | ILMN_1379152 | 0.21753703 | 0.09233443 | 0.00229732 | 0.61920876 | 0.01662501 | 0.04338819 |
| DPPA4 | ILMN_3155408 | 0.1739081 | 0.27023281 | 0.00088072 | 0.42732509 | 0.02642421 | 0.00022883 |
| D12WSU95E | ILMN_1224518 | 0.2458634 | 0.98949799 | 0.00168946 | 0.17924317 | 2.6261E−05 | 0.00064458 |
| 1700045I19RIK | ILMN_2967255 | 0.15347028 | 0.2851101 | 0.00069864 | 0.54346097 | 0.03092325 | 0.00360935 |
| MKI67 | ILMN_1222340 | 0.21845008 | 0.10582609 | 0.00151794 | 0.68757688 | 0.01387908 | 0.02193469 |
| HIRA | ILMN_1255703 | 0.69502846 | 0.47705474 | 0.00269556 | 0.18889994 | 0.00151539 | 1.6418E−05 |
| BC016608 | ILMN_2652989 | 0.50834338 | 0.24075668 | 0.00729255 | 0.54626521 | 0.00290472 | 0.01379723 |
| HIVEP3 | ILMN_3151547 | 0.6221573 | 0.5784284 | 0.00634728 | 0.98211123 | 0.02065996 | 0.00807037 |
| CDKN1B | ILMN_2638428 | 0.91702502 | 0.26092228 | 0.00094434 | 0.19107268 | 0.00076633 | 0.00462198 |
| TMEM146 | ILMN_1234738 | 0.55963286 | 0.26050993 | 0.01041221 | 0.53892459 | 0.00804008 | 0.02955068 |
| ELF2 | ILMN_1251296 | 0.62852774 | 0.99139746 | 0.01720939 | 0.55818707 | 0.01304328 | 0.00355372 |
| LOC100046343 | ILMN_1230976 | 0.92488823 | 0.46854285 | 0.01627632 | 0.46306215 | 0.0043081 | 0.01785105 |
| INSL6 | ILMN_2677859 | 0.64124773 | 0.60509358 | 0.01766838 | 0.95477043 | 0.00589193 | 0.00152369 |
| TSSC1 | ILMN_2457324 | 0.53469154 | 0.41014601 | 0.01120204 | 0.10387728 | 0.03441062 | 0.00068122 |
| APOM | ILMN_2769418 | 0.74805362 | 0.11640957 | 0.00163087 | 0.16942969 | 0.00116604 | 0.01142575 |
| BCORL1 | ILMN_2755424 | 0.50645326 | 0.13061814 | 0.00575317 | 0.4880663 | 0.02438683 | 0.0204965 |
| KAT2B | ILMN_1215305 | 0.8074212 | 0.6843084 | 0.01496102 | 0.43543792 | 0.00254275 | 0.00647192 |
| DPPA1 | ILMN_2656079 | 0.72920745 | 0.96338302 | 0.00390382 | 0.72649151 | 0.0030607 | 0.00151247 |
| LOC675899 | ILMN_1225187 | 0.69924634 | 0.55758262 | 0.00127133 | 0.94007528 | 0.00022169 | 7.6949E−07 |
| CDH23 | ILMN_1252019 | 0.50144466 | 0.43078095 | 0.00496081 | 0.98607056 | 0.03043863 | 0.01548972 |
| LOC384240 | ILMN_1231241 | 0.29257752 | 0.12197511 | 0.00135437 | 0.67015825 | 0.0059351 | 0.00826965 |
| PHIP | ILMN_1234202 | 0.05926837 | 0.35209449 | 0.03524732 | 0.20922543 | 1.6474E−05 | 0.00134681 |
| ZCCHC11 | ILMN_1231967 | 0.78809208 | 0.53552748 | 0.01222134 | 0.73732649 | 0.01180064 | 0.00751158 |
| C030004B10RIK | ILMN_1259439 | 0.73560437 | 0.81617257 | 0.00489393 | 0.8086789 | 0.01130307 | 0.00021935 |
| SH3BP2 | ILMN_1255766 | 0.62892589 | 0.10607381 | 0.00059545 | 0.22397233 | 0.00085081 | 0.01131408 |
| 9030003C19RIK | ILMN_2556478 | 0.36127662 | 0.22661649 | 0.00179321 | 0.79494852 | 0.01247801 | 0.01956102 |
| PTP4A2 | ILMN_2647282 | 0.34162233 | 0.6921419 | 0.04955706 | 0.0973134 | 0.00263027 | 0.0369895 |
| B230120H23RIK | ILMN_2595189 | 0.71928651 | 0.77212428 | 0.00553884 | 0.45416911 | 0.00849838 | 0.00055156 |
| LOC630421 | ILMN_1221849 | 0.05975928 | 0.20542135 | 0.00176669 | 0.40946695 | 0.04439239 | 0.00652537 |
| MYO5C | ILMN_1236641 | 0.77528015 | 0.09581598 | 0.00217533 | 0.18810176 | 0.01064048 | 0.04887721 |
| A130027D22RIK | ILMN_1233685 | 0.23873662 | 0.57185217 | 0.00277257 | 0.40997221 | 0.01542638 | 0.00142541 |
| TMPO | ILMN_1220051 | 0.52568721 | 0.50670076 | 0.01053137 | 0.1571467 | 0.00556007 | 0.03891587 |
| HSD362 | ILMN_1214838 | 0.30154913 | 0.39666725 | 0.01337088 | 0.8320674 | 0.02765843 | 0.0232042 |
| NDRG1 | ILMN_1250195 | 0.73937451 | 0.84400648 | 0.01386421 | 0.87075519 | 0.01268743 | 0.01261537 |
| RAB4A | ILMN_1212902 | 0.62659465 | 0.84220089 | 0.02522608 | 0.73571467 | 0.00865626 | 0.00969784 |
| RPL32 | ILMN_1232890 | 0.54424722 | 0.25994974 | 5.3471E−05 | 0.59562394 | 0.00027184 | 0.00061433 |
| A930025D01RIK | ILMN_1223523 | 0.55763739 | 0.65238929 | 0.01400344 | 0.1994279 | 0.02193567 | 0.00036436 |
| NUFIP2 | ILMN_2498813 | 0.5694042 | 0.12320942 | 0.00296033 | 0.37058243 | 0.01470444 | 0.03032341 |
| LOC236831 | ILMN_1253568 | 0.83421389 | 0.46981547 | 0.00142476 | 0.24689239 | 5.1009E−05 | 9.813E−05 |
| GPR12 | ILMN_1221410 | 0.37347225 | 0.17901589 | 0.00278232 | 0.72161404 | 0.01551884 | 0.01787435 |
| GLRA1 | ILMN_2657254 | 0.11641736 | 0.06158031 | 0.0008728 | 0.73302621 | 0.02180023 | 0.04600658 |
| TRPM1 | ILMN_3162921 | 0.51796388 | 0.33174608 | 0.00451316 | 0.06945752 | 0.01007648 | 5.2368E−05 |
| MIS12 | ILMN_2592174 | 0.68936945 | 0.60943265 | 0.03114273 | 0.92834338 | 0.00618323 | 0.00248934 |
| USP47 | ILMN_2455217 | 0.28202846 | 0.08191755 | 0.00143635 | 0.46809707 | 0.01035733 | 0.04837869 |
| DEFB14 | ILMN_3161733 | 0.215456 | 0.4928357 | 0.00668588 | 0.48780438 | 0.03891838 | 0.00874542 |
| 1600032L17RIK | ILMN_2524058 | 0.40281611 | 0.6061978 | 0.00612329 | 0.11938154 | 0.0179857 | 0.00027915 |
| HEATR5B | ILMN_2625626 | 0.57432396 | 0.27672303 | 0.0046491 | 0.64747954 | 0.01455003 | 0.01327886 |
| EP400 | ILMN_2575376 | 0.74679581 | 0.23641976 | 0.0061003 | 0.35341519 | 0.00094587 | 2.3443E−05 |
| C130075H21RIK | ILMN_1217814 | 0.43726261 | 0.82726663 | 0.00295087 | 0.24660914 | 0.03098716 | 0.0004333 |
| MYB | ILMN_2683910 | 0.33490693 | 0.61439991 | 0.00604983 | 0.58510712 | 0.0408895 | 0.00954837 |
| 2810402E24RIK | ILMN_1251628 | 0.10197727 | 0.1865506 | 0.00195351 | 0.64165857 | 0.03115129 | 0.01413344 |
| DLGAP2 | ILMN_2937486 | 0.76423206 | 0.74161064 | 0.0200202 | 0.47956738 | 0.01007235 | 0.014501 |
| COL23A1 | ILMN_2650447 | 0.77008033 | 0.99756857 | 0.00908151 | 0.71339762 | 0.00054938 | 0.00097296 |
| FAAH | ILMN_1233816 | 0.36353308 | 0.09357489 | 0.00697197 | 0.44021147 | 0.02065744 | 0.04644015 |
| DLC1 | ILMN_2648569 | 0.49025965 | 0.5212585 | 0.02576423 | 0.8837077 | 0.04649944 | 0.022319 |
| E230016F22RIK | ILMN_2571134 | 0.09802162 | 0.08109814 | 0.00164278 | 0.99182353 | 0.04202661 | 0.04124821 |
| 1700073E17RIK | ILMN_2449074 | 0.71043718 | 0.5882121 | 0.00122957 | 0.87952092 | 0.00052112 | 0.00048588 |
| MDM2 | ILMN_1250774 | 0.10703695 | 0.11493835 | 0.00154672 | 0.85678339 | 0.03934319 | 0.02735387 |
| E530016P10RIK | ILMN_1224376 | 0.2990389 | 0.74009471 | 0.00935998 | 0.29803596 | 0.04887577 | 0.0016525 |
| ZFP212 | ILMN_2521753 | 0.9554209 | 0.63285824 | 0.01285328 | 0.58371449 | 0.02154317 | 0.0014743 |
| IFI204 | ILMN_2623314 | 0.23940539 | 0.14068119 | 0.00361273 | 0.8595867 | 0.01870633 | 0.01379322 |
| TRPV3 | ILMN_2817797 | 0.24026546 | 0.25625841 | 0.00702167 | 0.96495267 | 0.0241875 | 0.03204151 |
| SEC1 | ILMN_2733335 | 0.74073227 | 0.89973033 | 0.04917371 | 0.76623156 | 0.03756973 | 0.01264343 |
| 9330133O14RIK | ILMN_2511527 | 0.87674031 | 0.31891103 | 0.01968785 | 0.46751448 | 0.04850705 | 0.03441428 |
| GALNT1 | ILMN_2625681 | 0.97148689 | 0.29753676 | 0.02327234 | 0.23909677 | 0.01812026 | 0.00037454 |
| FOXJ3 | ILMN_2650040 | 0.73644097 | 0.46665735 | 0.01420096 | 0.71064114 | 0.01271294 | 0.01178948 |
| 1810005K13RIK | ILMN_1246137 | 0.40557109 | 0.15106784 | 0.00714496 | 0.58012458 | 0.0198345 | 0.033473 |
| CLASP1 | ILMN_1254008 | 0.73119804 | 0.51506113 | 0.00177943 | 0.3022383 | 0.00176344 | 0.02302094 |
| 6030408K21RIK | ILMN_1218028 | 0.90502039 | 0.19355048 | 0.0009579 | 0.21228952 | 0.00084542 | 0.00383444 |
| A930008A22RIK | ILMN_2423730 | 0.08033924 | 0.65885651 | 0.00745835 | 0.12081829 | 8.1722E−06 | 0.00049256 |
| 1810007M14RIK | ILMN_2681311 | 0.98677383 | 0.32645363 | 0.01530257 | 0.27924789 | 0.01050978 | 0.04980064 |
| BC003331 | ILMN_2719794 | 0.25471202 | 0.58753271 | 0.01201949 | 0.54135948 | 0.02244509 | 0.01816724 |
| LOC382061 | ILMN_1245034 | 0.94469843 | 0.36699273 | 0.02143868 | 0.35145396 | 0.0090476 | 0.00143379 |
| NA112 | ILMN_2623754 | 0.73683778 | 0.14851426 | 0.01555081 | 0.25009623 | 0.01447008 | 0.02936269 |
| OLFR782 | ILMN_3161983 | 0.45044811 | 0.92625836 | 0.00590041 | 0.27142014 | 0.0111752 | 0.00024954 |
| CPEB2 | ILMN_1236346 | 0.75490198 | 0.71925691 | 0.00281149 | 0.46304434 | 0.00069739 | 0.0044823 |
| 2510049I19RIK | ILMN_2778799 | 0.91464963 | 0.41628174 | 0.00811012 | 0.43802335 | 0.00553053 | 0.02647587 |
| LCN3 | ILMN_1236379 | 0.39628467 | 0.40563118 | 0.00857505 | 0.92702367 | 0.03849304 | 0.02718766 |
| 9930021J03RIK | ILMN_2626694 | 0.84731035 | 0.41953277 | 0.00968591 | 0.28164636 | 0.00505829 | 0.0227078 |
| LOC268939 | ILMN_1215666 | 0.97166046 | 0.23740673 | 0.01049384 | 0.14453449 | 0.00091119 | 0.04187746 |
| IGHV14S3_{—} | ILMN_2453303 | 0.22474706 | 0.1772146 | 0.01131447 | 0.96094783 | 9.7043E−05 | 1.9202E−05 |
| X03573$M12991X03573_{—} | | | | | | | |
| IG_HEAVY_{—} | | | | | | | |
| VARIABLE_14S3_203 | | | | | | | |
| 4930524L23RIK | ILMN_2610591 | 0.97633163 | 0.44405621 | 0.00316642 | 0.44146577 | 0.00327003 | 0.00507241 |
| NANP | ILMN_2629922 | 0.4439226 | 0.33542528 | 0.00272161 | 0.86697218 | 0.01347946 | 0.01328289 |
| PCSK2 | ILMN_1244247 | 0.98271261 | 0.5580062 | 0.01718032 | 0.54541442 | 0.01222075 | 0.01500895 |
| C87414 | ILMN_1243166 | 0.83011253 | 0.58061946 | 0.02051233 | 0.38781839 | 0.00492303 | 0.03676842 |
| TMEFF2 | ILMN_2518100 | 0.5540378 | 0.30736402 | 0.00549964 | 0.67788762 | 0.02123168 | 0.03470806 |
| MIA3 | ILMN_2600646 | 0.86291188 | 0.50022412 | 0.01001686 | 0.619891 | 0.00607289 | 0.00032584 |
| EG546913 | ILMN_2531690 | 0.83003528 | 0.83407704 | 0.00102345 | 0.96611446 | 0.00064849 | 0.00010015 |
| A630040J04RIK | ILMN_1252015 | 0.48378882 | 0.87063997 | 0.00320099 | 0.52722591 | 0.00042433 | 0.00089277 |
| HARS | ILMN_2769030 | 0.48614677 | 0.31401417 | 0.01130818 | 0.80919235 | 0.01327593 | 0.01178117 |
| HRK | ILMN_1229407 | 0.2096328 | 0.12231789 | 0.00288475 | 0.89534884 | 0.03224908 | 0.02746623 |
| LOC100045437 | ILMN_1228965 | 0.75055515 | 0.92688503 | 0.01007045 | 0.79290195 | 0.00777848 | 0.00618076 |
| AFMID | ILMN_2702894 | 0.73491375 | 0.50553765 | 0.01521108 | 0.77275529 | 0.04804283 | 0.04613881 |
| PMS1 | ILMN_1250517 | 0.63673516 | 0.71973317 | 0.02227416 | 0.89567579 | 0.02348795 | 0.02254558 |
| ID2 | ILMN_2887239 | 0.87028953 | 0.47511409 | 0.02798367 | 0.54217128 | 0.0158696 | 0.04312451 |
| FABP2 | ILMN_2706493 | 0.80337467 | 0.78665036 | 0.01344875 | 0.98455719 | 0.00177294 | 0.00177906 |
| ATG12 | ILMN_2663570 | 0.27659474 | 0.66933767 | 0.00221969 | 0.41535757 | 0.02996064 | 0.00364526 |
| FBXW13 | ILMN_2625152 | 0.55067562 | 0.60422372 | 0.01436917 | 0.88202557 | 0.03730302 | 0.01508965 |
| TDH | ILMN_1244724 | 0.29912682 | 0.22027664 | 0.00109699 | 0.86469091 | 0.02808824 | 0.03722901 |
| CALCRL | ILMN_1235390 | 0.61928835 | 0.95951597 | 0.03885534 | 0.51870583 | 0.03448207 | 0.0042841 |
| IL1F9 | ILMN_2658704 | 0.95996795 | 0.36258599 | 0.0027602 | 0.32615103 | 0.00456295 | 0.01073443 |
| SYT10 | ILMN_2724250 | 0.36728496 | 0.72200844 | 0.01447158 | 0.43001287 | 0.04043248 | 0.0041577 |
| MEX3A | ILMN_2479681 | 0.33800926 | 0.69572258 | 0.02090267 | 0.48891534 | 0.02248677 | 0.01516916 |
| PRICKLE2 | ILMN_3015864 | 0.84678228 | 0.78740959 | 0.02091569 | 0.96913695 | 0.01472913 | 0.00345005 |
| 5830415L20RIK | ILMN_1257675 | 0.976909 | 0.94073976 | 0.01481662 | 0.90901729 | 0.02131485 | 0.00256728 |
| GAPVD1 | ILMN_1238871 | 0.63675362 | 0.13555767 | 0.00544842 | 0.27193364 | 0.00501968 | 0.02911601 |
| LOC381770 | ILMN_2539337 | 0.42759367 | 0.73766238 | 0.0151302 | 0.56396291 | 0.03833058 | 0.01488009 |
| TATDN3 | ILMN_2647204 | 0.77448651 | 0.23572372 | 0.00130565 | 0.29300657 | 0.00044483 | 0.02435293 |
| KRT32 | ILMN_2895346 | 0.83250256 | 0.51569874 | 0.00650346 | 0.63950501 | 0.00114191 | 0.00010869 |
| LRRC18 | ILMN_2728504 | 0.89493152 | 0.59100111 | 0.00523404 | 0.66901961 | 0.00317902 | 0.00079243 |
| NIN | ILMN_2778195 | 0.68032543 | 0.98972382 | 0.00937265 | 0.58827183 | 0.00170877 | 0.00140829 |
| 5830411J07RIK | ILMN_2679267 | 0.91687524 | 0.57076878 | 0.00800807 | 0.63502177 | 0.00951029 | 0.01081438 |
| EEFSEC | ILMN_2587281 | 0.78972673 | 0.9248816 | 0.01022288 | 0.67206418 | 0.0051822 | 0.00327079 |
| SPDYB | ILMN_2756787 | 0.41881968 | 0.32985568 | 0.01273211 | 0.93027948 | 0.0425628 | 0.0364783 |
| RNF150 | ILMN_3032501 | 0.10404093 | 0.24949732 | 0.02284489 | 0.34832122 | 8.5477E−05 | 7.2293E−05 |
| SVNGR4 | ILMN_3070294 | 0.58603374 | 0.74678443 | 0.01678942 | 0.77844292 | 0.04810332 | 0.01950989 |
| 6030408816RIK | ILMN_3125764 | 0.37545518 | 0.38985728 | 0.00402912 | 0.88443972 | 0.03648602 | 0.01495938 |
| C130039E17RIK | ILMN_2494727 | 0.2570242 | 0.94446193 | 0.00747845 | 0.19657178 | 0.0186735 | 0.00127309 |
| KRIT1 | ILMN_2701155 | 0.29754524 | 0.12428278 | 0.01525668 | 0.66355969 | 0.00094451 | 8.5201E−05 |
| control_ILMN_1378774 | ILMN_1378774 | 0.93075559 | 0.28610953 | 0.03628321 | 0.20533938 | 0.02992914 | 0.00197571 |
| PAPPA | ILMN_2630646 | 0.37572351 | 0.24694961 | 0.0127078 | 0.79497675 | 0.03130728 | 0.04376852 |
| 1700016L04RIK | ILMN_2499229 | 0.90685361 | 0.3496515 | 0.00372799 | 0.28777419 | 0.00681981 | 0.03561193 |
| C130072C03RIK | ILMN_1233936 | 0.80838551 | 0.83984442 | 0.01171633 | 0.62099255 | 0.00449699 | 0.01414307 |
| OLFR564 | ILMN_2771303 | 0.60592345 | 0.36175661 | 0.00386213 | 0.7522095 | 0.00850738 | 0.00363427 |
| 1700016G05RIK | ILMN_2637903 | 0.70654921 | 0.58625198 | 0.03809295 | 0.89807309 | 0.00870429 | 0.00246756 |
| RgenericRIP1 | ILMN_2681694 | 0.52726949 | 0.44369673 | 0.0030417 | 0.9475417 | 0.02276766 | 0.01581923 |
| K20D4 | ILMN_1240743 | 0.96207944 | 0.43763372 | 0.00162244 | 0.38849163 | 0.00191142 | 0.00558935 |
| 4922502D21RIK | ILMN_2593944 | 0.4155873 | 0.26085025 | 0.00141644 | 0.80943674 | 0.02878727 | 0.03578821 |
| GM949 | ILMN_2531100 | 0.75549241 | 0.57398859 | 0.02584055 | 0.812046 | 0.02768367 | 0.02515704 |
| DLG4 | ILMN_2710764 | 0.88945477 | 0.63134593 | 0.00987864 | 0.71949702 | 0.00922123 | 0.01399519 |
| PTPN22 | ILMN_2766313 | 0.89147187 | 0.39788189 | 0.00599815 | 0.2959911 | 0.0071583 | 0.00020474 |
| MEDI | ILMN_2695175 | 0.69784662 | 0.3155412 | 0.00611454 | 0.51360092 | 0.01261526 | 0.04895724 |
| HPN | ILMN_1240252 | 0.70375464 | 0.84003357 | 0.02512481 | 0.46934315 | 0.01165401 | 0.00317289 |
| USP47 | ILMN_1256774 | 0.8866563 | 0.08427609 | 0.00149343 | 0.06085008 | 0.00993913 | 2.7829E−05 |
| CTDSPL2 | ILMN_3154869 | 0.87357331 | 0.45992752 | 0.01471252 | 0.56168486 | 0.01511923 | 0.00170494 |
| PARP11 | ILMN_1221241 | 0.89036915 | 0.66385821 | 0.00113541 | 0.56321863 | 0.00478319 | 0.01014818 |
| LOC381561 | ILMN_2536925 | 0.17237484 | 0.21686297 | 0.00759509 | 0.64649338 | 0.04390595 | 0.00930537 |
| V1RD9 | ILMN_2959825 | 0.97881914 | 0.13175262 | 0.00690149 | 0.09799273 | 0.00570138 | 5.8945E−06 |
| SPN | ILMN_1255969 | 0.55964734 | 0.71117142 | 0.00751623 | 0.85787049 | 0.00950215 | 0.01371046 |
| KCNK4 | ILMN_2625370 | 0.6927491 | 0.86587608 | 0.04472393 | 0.50165892 | 0.00640631 | 0.02599097 |
| PAX6 | ILMN_2504978 | 0.69025478 | 0.25190942 | 0.01354257 | 0.08344413 | 0.00354709 | 0.0395486 |
| COPS3 | ILMN_1218678 | 0.51693211 | 0.63688322 | 0.02373088 | 0.20247631 | 0.00501893 | 0.02654163 |
| OLFR894 | ILMN_2631775 | 0.55832722 | 0.99847092 | 0.04395964 | 0.42135784 | 0.00330976 | 0.00792789 |
| 9530053H22 | ILMN_2617639 | 0.41889249 | 0.35405494 | 0.00357814 | 0.05425697 | 0.00030686 | 0.01195699 |
| 5830472H07RIK | ILMN_2755293 | 0.36501638 | 0.66607508 | 0.02046626 | 0.1182634 | 0.00039784 | 0.02746328 |
| D930007M19RIK | ILMN_2581178 | 0.76339543 | 0.60604606 | 0.02241012 | 0.33537703 | 0.00451539 | 0.01729312 |
| D130078K04RIK | ILMN_2569683 | 0.66636721 | 0.25734967 | 0.04135106 | 0.05132471 | 0.02878696 | 0.00014926 |
| LOC384287 | ILMN_1226110 | 0.54388276 | 0.58141105 | 0.00333332 | 0.19201685 | 0.01748532 | 0.00017648 |
| LOC100048049 | ILMN_2708610 | 0.95218128 | 0.25590685 | 0.01148101 | 0.21123154 | 0.0108032 | 0.04933477 |
| 1700112H15RIK | ILMN_2743541 | 0.64861495 | 0.3201502 | 0.0108812 | 0.10933982 | 0.02115428 | 0.00027934 |
| RNF17 | ILMN_2741929 | 0.71345974 | 0.30390726 | 0.00557822 | 0.17470226 | 0.01156905 | 0.02734088 |
| NARF | ILMN_1244845 | 0.32813687 | 0.07214624 | 0.01761269 | 0.41766052 | 0.00279047 | 0.00015962 |
| HSPA1B | ILMN_2530672 | 0.77146596 | 0.98054272 | 0.0373104 | 0.70191869 | 0.01984534 | 0.017804 |
| B930071N01RIK | ILMN_1247868 | 0.96323283 | 0.64426691 | 0.00627488 | 0.65225828 | 0.00409529 | 0.00147182 |
| SEMA3E | ILMN_1228525 | 0.63039278 | 0.32434364 | 0.00681 | 0.60546066 | 0.00583882 | 0.00908435 |
| GALNT6 | ILMN_2627386 | 0.81446882 | 0.7918429 | 0.02845447 | 0.54047129 | 0.02603685 | 0.00196729 |
| NCOA2 | ILMN_2573793 | 0.8386801 | 0.97919008 | 0.04731113 | 0.81134649 | 0.03878777 | 0.01017342 |
| LOC385027 | ILMN_1255900 | 0.65753886 | 0.48289369 | 0.00956486 | 0.22419555 | 0.02822087 | 0.0016544 |
| SPRR2K | ILMN_2990390 | 0.46106991 | 0.19883289 | 0.02020776 | 0.5001156 | 0.00077923 | 0.000363 |
| STAT4 | ILMN_2792599 | 0.90331319 | 0.92233814 | 0.01253158 | 0.81432899 | 0.00936326 | 0.01566569 |
| 1700006H02RIK | ILMN_1253202 | 0.71958726 | 0.49588584 | 0.00439927 | 0.77777038 | 0.02141138 | 0.01990318 |
| OLFR463 | ILMN_2702643 | 0.941253 | 0.95422277 | 0.04609905 | 0.87367146 | 0.02446877 | 0.01626319 |
| LOC637082 | ILMN_1233896 | 0.97759418 | 0.77459971 | 0.02442676 | 0.72223266 | 0.01332385 | 0.0034332 |
| CHMP7 | ILMN_2588610 | 0.72596566 | 0.6457792 | 0.01639739 | 0.9779851 | 0.00470516 | 0.00044208 |
| AGPAT5 | ILMN_1250939 | 0.96146142 | 0.57031058 | 0.02382884 | 0.57463699 | 0.01575584 | 0.01665907 |
| GPRIN3 | ILMN_2722319 | 0.71936877 | 0.40942604 | 0.0162081 | 0.17317377 | 0.00482278 | 0.02236757 |
| R3HDM1 | ILMN_2759868 | 0.51275284 | 0.82741759 | 0.01392116 | 0.30805784 | 0.04307204 | 0.0024589 |
| GM239 | ILMN_2814357 | 0.85644322 | 0.36077622 | 0.01078965 | 0.2673967 | 0.01257121 | 0.02139083 |
| genericC | ILMN_2606088 | 0.36934443 | 0.14213324 | 0.00634593 | 0.62058965 | 0.00072505 | 4.2492E−05 |
| GUCY2C | ILMN_2911551 | 0.93114386 | 0.6791989 | 0.02669526 | 0.72742603 | 0.01922554 | 0.01909273 |
| DNAJC28 | ILMN_2592571 | 0.79527431 | 0.92289593 | 0.02298836 | 0.66107013 | 0.00609899 | 0.00449318 |
| A630057J21RIK | ILMN_1222357 | 0.23149645 | 0.69829606 | 0.0121469 | 0.05686233 | 0.00045583 | 0.01158149 |
| PDK2 | ILMN_2737658 | 0.91074165 | 0.61606209 | 0.04487631 | 0.491808 | 0.02485846 | 0.04404716 |
| 1110032A03RIK | ILMN_1215043 | 0.60423819 | 0.77153705 | 0.00539919 | 0.77634877 | 0.01693617 | 0.00576525 |
| 2310016E02RIK | ILMN_1254063 | 0.70483626 | 0.65410085 | 0.01083083 | 0.95255869 | 0.01467721 | 0.02001851 |
| C330011J12RIK | ILMN_2615978 | 0.85869838 | 0.92011073 | 0.01561237 | 0.7486874 | 0.0019331 | 0.00520559 |
| JRKL | ILMN_1255837 | 0.94272483 | 0.80292788 | 0.04124635 | 0.84524612 | 0.02315801 | 0.01944571 |
| ZFP120 | ILMN_2456317 | 0.86261706 | 0.86740893 | 0.01075473 | 0.98911924 | 0.00926091 | 0.00636258 |
| KISS1 | ILMN_2735829 | 0.76581773 | 0.21618646 | 0.00727701 | 0.11170053 | 0.02023543 | 0.00019085 |
| RTN3 | ILMN_2572492 | 0.5310507 | 0.71969456 | 0.02242047 | 0.73699268 | 0.0375872 | 0.019464 |
| AW822252 | ILMN_2744956 | 0.91107588 | 0.66282674 | 0.02595894 | 0.48769444 | 0.00148654 | 0.01190856 |
| LRRC39 | ILMN_1230043 | 0.78452833 | 0.88892882 | 0.01360738 | 0.88972638 | 0.00158734 | 0.00430995 |
| SCNN1B | ILMN_2618383 | 0.89820542 | 0.82246064 | 0.03437774 | 0.66818803 | 0.01921091 | 0.00309561 |
| DLX6 | ILMN_1213404 | 0.98659216 | 0.69950278 | 0.01383192 | 0.69768922 | 0.01328586 | 0.00162742 |
| GM438 | ILMN_1248574 | 0.29999719 | 0.55050151 | 0.0105011 | 0.65192125 | 0.04085148 | 0.02498608 |
| LOC236397 | ILMN_1239998 | 0.68197029 | 0.82258913 | 0.02341937 | 0.46093245 | 0.0237734 | 0.00558543 |
| LOC381448 | ILMN_2528515 | 0.78623555 | 0.33559508 | 0.0078061 | 0.517919 | 0.0070339 | 0.00022804 |
| CCDC104 | ILMN_2643580 | 0.55123773 | 0.70680186 | 0.0074226 | 0.2389928 | 0.00245492 | 0.00360147 |
| LOC279638 | ILMN_2588075 | 0.64349514 | 0.82890664 | 0.01900655 | 0.43850016 | 0.00446086 | 0.01666119 |
| MADD | ILMN_1256376 | 0.88413835 | 0.5873814 | 0.00493436 | 0.69788059 | 0.01748976 | 0.02498916 |
| LOC333749 | ILMN_2530361 | 0.9961254 | 0.94549994 | 0.00891057 | 0.94341468 | 0.01926402 | 0.0087053 |
| AGT | ILMN_1227398 | 0.98389935 | 0.71082852 | 0.03176339 | 0.68226388 | 0.03100151 | 0.03795007 |
| C77370 | ILMN_1218340 | 0.6355803 | 0.74538896 | 0.00808846 | 0.35934138 | 0.00339863 | 0.00443228 |
| LOC382454 | ILMN_1255829 | 0.96588171 | 0.73772708 | 0.01511148 | 0.75778174 | 0.01698715 | 0.02638877 |
| 9130601C02RIK | ILMN_1216153 | 0.41100887 | 0.94744117 | 0.00520864 | 0.26716372 | 0.04481248 | 0.00079179 |
| B230216G23RIK | ILMN_2481643 | 0.57384028 | 0.48180684 | 0.00610837 | 0.16323616 | 0.00056974 | 0.02624308 |
| BMPR1B | ILMN_1219486 | 0.46145933 | 0.66822667 | 0.013751 | 0.69879477 | 0.00500677 | 0.00545184 |
| LOC100046568 | ILMN_1258903 | 0.3199598 | 0.56576602 | 0.04571049 | 0.50062543 | 0.00563038 | 0.00431517 |
| A130003P22RIK | ILMN_1257646 | 0.71247927 | 0.82191867 | 0.0033766 | 0.85251926 | 0.00438661 | 0.00210537 |
| CFL2 | ILMN_1249976 | 0.6912082 | 0.47018116 | 0.01946891 | 0.77798251 | 0.04355008 | 0.0357747 |
| control_ILMN_1378775 | ILMN_1378775 | 0.24499389 | 0.26320839 | 0.03290204 | 0.81180565 | 0.00164824 | 0.00063354 |
| TCFAP2C | ILMN_2783225 | 0.76201807 | 0.94087475 | 0.02807743 | 0.80847181 | 0.01136225 | 0.02645559 |
| BHLHB3 | ILMN_2759227 | 0.59822063 | 0.90684222 | 0.01459628 | 0.46655189 | 0.02520291 | 0.00413327 |
| UGDH | ILMN_1253090 | 0.80637062 | 0.91996988 | 0.00850688 | 0.86603197 | 0.02134136 | 0.00948229 |
| control_ILMN_1379205 | ILMN_1379205 | 0.76025905 | 0.98196255 | 0.02374306 | 0.71823575 | 0.04033738 | 0.01453797 |
| CRLF1 | ILMN_2747811 | 0.79797247 | 0.28989628 | 0.00239399 | 0.1858294 | 0.00732642 | 0.03905401 |
| SOX2 | ILMN_1248711 | 0.77498 | 0.64492465 | 0.02193392 | 0.86465206 | 0.02410687 | 0.03050535 |
| LOC229152 | ILMN_1240991 | 0.75807476 | 0.52647195 | 0.00704108 | 0.84498087 | 0.03417437 | 0.0058696 |
| 2210008F06RIK | ILMN_2429915 | 0.91583231 | 0.97857251 | 0.04923649 | 0.87185551 | 0.03416033 | 0.01793713 |
| 9530013H16R1K | ILMN_2559067 | 0.57713891 | 0.32076174 | 0.01677346 | 0.72134189 | 0.04932107 | 0.04860174 |
| MACROD2 | ILMN_1226796 | 0.71709711 | 0.44062902 | 0.02055885 | 0.71708219 | 0.02601735 | 0.01654778 |
| ZBTB44 | ILMN_2589136 | 0.48704466 | 0.63544273 | 0.01313125 | 0.75239037 | 0.04857492 | 0.01537547 |
| LOC209474 | ILMN_1245275 | 0.79098578 | 0.95793058 | 0.01722142 | 0.72365671 | 0.01473191 | 0.0078102 |
| OLFR1158 | ILMN_1254931 | 0.77110867 | 0.89502054 | 0.02695462 | 0.61488634 | 0.03490769 | 0.00534404 |
| LOC383612 | ILMN_1245739 | 0.96088752 | 0.49445505 | 0.0133369 | 0.51403628 | 0.01234276 | 0.00789858 |
| NFAT5 | ILMN_2732123 | 0.72207089 | 0.63730457 | 0.01379799 | 0.37553032 | 0.00519688 | 0.03425991 |
| IGHVS515_AF290966_{—} | ILMN_2432375 | 0.23326177 | 0.59391903 | 0.00827536 | 0.05248274 | 0.00034004 | 0.0201746 |
| IG_HEAVY_{—} | | | | | | | |
| VARIABLE_5515_49 | | | | | | | |
| ELMO2 | ILMN_3058341 | 0.12486628 | 0.24567253 | 0.03204461 | 0.65701953 | 0.00045446 | 0.00128682 |
| 3110079O15RIK | ILMN_2598181 | 0.69113605 | 0.35355466 | 0.00161013 | 0.17857553 | 0.00258872 | 0.01840637 |
| GFRA3 | ILMN_3029078 | 0.62264403 | 0.92745727 | 0.01762037 | 0.65173589 | 0.03892889 | 0.02104455 |
| OSBPL6 | ILMN_1219286 | 0.42124404 | 0.42368156 | 0.01623477 | 0.93862005 | 0.00172301 | 0.0008632 |
| DLG1 | ILMN_2763839 | 0.73896101 | 0.80437453 | 0.04979664 | 0.87479492 | 0.0096557 | 0.00635505 |
| D130063P19RIK | ILMN_1238936 | 0.94809579 | 0.84171066 | 0.02702422 | 0.89126407 | 0.0207869 | 0.0134556 |
| EG634762 | ILMN_1214492 | 0.26319253 | 0.79259245 | 0.01735231 | 0.10089143 | 0.0003285 | 0.00958642 |
| 4833419KD8RIK | ILMN_1250504 | 0.90497419 | 0.74215973 | 0.02042767 | 0.81072135 | 0.0042437 | 0.00196065 |
| D130061E05RIK | ILMN_2573307 | 0.70946172 | 0.61405852 | 0.0316363 | 0.97138412 | 0.03799442 | 0.00999837 |
| TMEM219 | ILMN_2689569 | 0.2764255 | 0.72626184 | 0.03771418 | 0.43175872 | 0.00116995 | 0.00879276 |
| OA1 | ILMN_1242255 | 0.48716604 | 0.64452628 | 0.02998572 | 0.13614133 | 0.0012152 | 0.0120236 |
| OLFR1180 | ILMN_2649883 | 0.82220384 | 0.65018321 | 0.03332559 | 0.83964329 | 0.04497682 | 0.03216904 |
| EFCAB1 | ILMN_2917280 | 0.40779353 | 0.73175186 | 0.00740283 | 0.59835212 | 0.00516637 | 0.01054621 |
| control_ILMN_1378664 | ILMN_1378664 | 0.73998782 | 0.57317844 | 0.04875388 | 0.84376255 | 0.03733169 | 0.01335162 |
| BRPF1 | ILMN_2800859 | 0.10317996 | 0.45657197 | 0.01871488 | 0.159891 | 0.00037443 | 0.00052146 |
| CORO1B | ILMN_2788546 | 0.61023246 | 0.91171296 | 0.01479403 | 0.48331453 | 0.0055325 | 0.01554437 |
| LOC235971 | ILMN_1247372 | 0.52222184 | 0.34554539 | 0.01442529 | 0.7525038 | 0.02629384 | 0.04443921 |
| LOC383940 | ILMN_2536510 | 0.77825561 | 0.94255106 | 0.04170942 | 0.6416792 | 0.00535638 | 0.01023982 |
| RNF207 | ILMN_1237330 | 0.15249074 | 0.74859334 | 0.03681339 | 0.20400522 | 0.00054508 | 0.00924396 |
| 1700081L16RIK | ILMN_2426180 | 0.4254824 | 0.3879785 | 0.02017245 | 0.97625438 | 0.03680693 | 0.04755114 |
| OLFR750 | ILMN_2773535 | 0.99805882 | 0.34741304 | 0.00424471 | 0.37021352 | 0.01764381 | 0.03092697 |
| LOC385626 | ILMN_1244702 | 0.86191682 | 0.90550993 | 0.02123738 | 0.74013397 | 0.01979016 | 0.0142778 |
| LOC384513 | ILMN_1230132 | 0.9775506 | 0.78321033 | 0.03643822 | 0.78378945 | 0.01961725 | 0.01792928 |
| LOC385059 | ILMN_2542073 | 0.83322545 | 0.64867558 | 0.01307523 | 0.81791118 | 0.02015499 | 0.01508891 |
| 4930571C24RIK | ILMN_1228034 | 0.8721725 | 0.44826867 | 0.03700555 | 0.54621985 | 0.03758677 | 0.03673126 |
| PPP1R14D | ILMN_1253015 | 0.8287285 | 0.44941136 | 0.0208985 | 0.25160589 | 0.00295591 | 0.03600523 |
| GJA7 | ILMN_1253346 | 0.80834514 | 0.75164716 | 0.02893306 | 0.94686025 | 0.04593948 | 0.04219593 |
| OLFR1085 | ILMN_1248151 | 0.61996214 | 0.86465087 | 0.04986154 | 0.69712713 | 0.03196955 | 0.03620173 |
| NEUROD4 | ILMN_2766939 | 0.94689433 | 0.9900661 | 0.01556886 | 0.95536905 | 0.01714781 | 0.01308462 |
| LTBP1 | ILMN_1224967 | 0.90429089 | 0.60644319 | 0.04980213 | 0.6815483 | 0.04443474 | 0.01511683 |
| control_ILMN_1378571 | ILMN_1378571 | 0.99545055 | 0.73862332 | 0.04751979 | 0.72098434 | 0.04519215 | 0.02180431 |
| PIK3R4 | ILMN_2543088 | 0.75550272 | 0.7071606 | 0.03348288 | 0.45137917 | 0.01998057 | 0.04492477 |
| 2210003I03RIK | ILMN_2710359 | 0.70593469 | 0.85165753 | 0.02261457 | 0.86900023 | 0.03307202 | 0.03281985 |
| 29D0003A17RIK | ILMN_2449674 | 0.96480497 | 0.60371744 | 0.04840899 | 0.6149156 | 0.03532408 | 0.00511049 |
| EZH2 | ILMN_2606429 | 0.88387031 | 0.652619 | 0.04909333 | 0.77133926 | 0.03376462 | 0.00590324 |
| BTF3L4 | ILMN_2658543 | 0.92055293 | 0.79662617 | 0.02408994 | 0.86240269 | 0.01294046 | 0.01870436 |
| LOC676672 | ILMN_1236130 | 0.79366788 | 0.42669627 | 0.0324377 | 0.5855754 | 0.01847081 | 0.00319123 |
| control_ILMN_1379333 | ILMN_1379333 | 0.12888566 | 0.9453906 | 0.03593319 | 0.10594139 | 7E−05 | 0.01310016 |
| B230323D24RIK | ILMN_1213297 | 0.74482804 | 0.95530433 | 0.02591804 | 0.61767759 | 0.03216558 | 0.00373812 |
| 3110052M02RIK | ILMN_2463970 | 0.85112691 | 0.54323523 | 0.02464873 | 0.37974163 | 0.01307558 | 0.02624936 |
| ENSMUSG00000071316 | ILMN_1231109 | 0.87211247 | 0.98926339 | 0.04433271 | 0.85279003 | 0.0362554 | 0.01867314 |
| LOC384215 | ILMN_2537640 | 0.17703635 | 0.72581643 | 0.02350565 | 0.16159152 | 0.0009221 | 0.00298547 |
| D530031C13RIK | ILMN_1217757 | 0.98471016 | 0.67782315 | 0.04540006 | 0.6685376 | 0.03616971 | 0.04868737 |
| LOC277422 | ILMN_2527881 | 0.89433054 | 0.75733563 | 0.02984326 | 0.86561313 | 0.04122515 | 0.03448776 |
| A530088H08RIK | ILMN_2601705 | 0.73111733 | 0.39411525 | 0.04498969 | 0.65159504 | 0.03093272 | 0.00356921 |
| HEATR5A | ILMN_2694279 | 0.7621217 | 0.78255045 | 0.04556355 | 0.92636041 | 0.02912756 | 0.01002533 |
| 6430527G18RIK | ILMN_2754183 | 0.13974324 | 0.66424425 | 0.02858616 | 0.17640457 | 0.00030636 | 0.00397781 |
| TRPC6 | ILMN_2446212 | 0.92191975 | 0.71619721 | 0.02563041 | 0.62175009 | 0.03298053 | 0.0056644 |
| GM362 | ILMN_1222972 | 0.66428504 | 0.44599883 | 0.0342508 | 0.77150163 | 0.01049321 | 0.00192869 |
| PCDHGA8 | ILMN_2705452 | 0.86448939 | 0.97817948 | 0.03578371 | 0.83418737 | 0.03307592 | 0.03874447 |
| LOC620009 | ILMN_2635082 | 0.46415035 | 0.22663146 | 0.03446288 | 0.52707304 | 0.00033295 | 0.00025567 |
| LOC100043389 | ILMN_1256414 | 0.33651119 | 0.7213683 | 0.01170848 | 0.42963795 | 0.00178557 | 0.00209377 |
| SMARCA2 | ILMN_1252932 | 0.68251403 | 0.88059928 | 0.01160248 | 0.53929361 | 0.01209175 | 0.02064582 |
| A230066D03RIK | ILMN_2694557 | 0.74782694 | 0.6233261 | 0.03082403 | 0.87425008 | 0.03790339 | 0.032595 |
| UTX | ILMN_2579537 | 0.89905619 | 0.98414172 | 0.04612245 | 0.85931279 | 0.02447713 | 0.01442723 |
| OLFR1230 | ILMN_2662149 | 0.91334388 | 0.68532674 | 0.02142457 | 0.58031462 | 0.03000352 | 0.00449387 |
| CREBZF | ILMN_1220787 | 0.64191023 | 0.56581421 | 0.03441167 | 0.23558574 | 0.00434827 | 0.04992517 |
| RARRES1 | ILMN_2605971 | 0.95750188 | 0.51989413 | 0.04960964 | 0.43597227 | 0.0341128 | 0.00522452 |
| D130050A01RIK | ILMN_1215837 | 0.85818244 | 0.65897013 | 0.00758375 | 0.79388925 | 0.01433698 | 0.01656402 |
| LOC382783 | ILMN_1258648 | 0.79545382 | 0.83993882 | 0.02696047 | 0.91754583 | 0.01942506 | 0.00864141 |
| C430020E23RIK | ILMN_1221998 | 0.34147765 | 0.6328869 | 0.04174783 | 0.61859671 | 0.00272655 | 0.01621384 |
| ADAMTSL3 | ILMN_1239668 | 0.33183121 | 0.75723625 | 0.01454444 | 0.46400486 | 0.00409622 | 0.01295871 |
| SCL0070954.1_214 | ILMN_2425193 | 0.83860572 | 0.63897727 | 0.04612875 | 0.42983495 | 0.03311454 | 0.00513378 |
| PTCHD2 | ILMN_1239751 | 0.98881206 | 0.86022771 | 0.04418891 | 0.85733836 | 0.0257898 | 0.01624929 |
| CNTN2 | ILMN_1249156 | 0.66466561 | 0.52521167 | 0.02574904 | 0.83385971 | 0.00534851 | 0.00370346 |
| 6720487G11RIK | ILMN_2649435 | 0.96481458 | 0.72365037 | 0.03056654 | 0.73355765 | 0.01492367 | 0.00837893 |
| SERPINB3D | ILMN_3009747 | 0.75326559 | 0.83609816 | 0.0413928 | 0.51010543 | 0.00875595 | 0.01225013 |
| CRAT | ILMN_1220786 | 0.40829488 | 0.94468035 | 0.04796514 | 0.39169148 | 0.01052467 | 0.0423267 |
| 4921522P10RIK | ILMN_1218156 | 0.55741306 | 0.51678788 | 0.04020993 | 0.90899286 | 0.02851827 | 0.0058245 |
| A830021M18 | ILMN_2595299 | 0.84943491 | 0.92235962 | 0.04522046 | 0.8981181 | 0.02975733 | 0.01580216 |
| RTN2 | ILMN_1250904 | 0.09150444 | 0.43211752 | 0.03209568 | 0.29505476 | 0.00103809 | 0.00721221 |
| RPL10A | ILMN_2902115 | 0.82821072 | 0.71019462 | 0.00906988 | 0.87798018 | 0.01490853 | 0.01824741 |
| LOC382760 | ILMN_2535747 | 0.925786 | 0.74325413 | 0.04228212 | 0.65721189 | 0.04681705 | 0.0425333 |
| 4930543E05RIK | ILMN_2584057 | 0.63273143 | 0.91424409 | 0.03208089 | 0.64543484 | 0.02010738 | 0.01645552 |
| 5730599A22RIK | ILMN_1212946 | 0.44726235 | 0.9680857 | 0.04285622 | 0.410001 | 0.01256541 | 0.02652421 |

UP IN GA AND REF ONLY

| | | | | | | | |
|---|---|---|---|---|---|---|---|
| C130079K02RIK | ILMN_2568571 | 1.0414E−12 | 1.1619E−10 | 0.12810055 | 0.7257423 | 9.9884E−07 | 2.0124E−05 |
| ALS2CL | ILMN_1240592 | 1.6747E−05 | 7.0707E−06 | 0.25292734 | 0.97285087 | 0.01087249 | 0.00804871 |
| 2810410P22RIK | ILMN_2692986 | 1.1279E−08 | 1.3379E−09 | 0.08032716 | 0.34642833 | 9.0516E−05 | 0.00016472 |
| HNRNPK | ILMN_1245987 | 4.2889E−05 | 0.0002067 | 0.14135617 | 0.62563128 | 0.00414261 | 0.04211374 |
| TRAT1 | ILMN_2652867 | 1.0482E−09 | 5.4608E−10 | 0.69415784 | 0.50002267 | 1.2723E−07 | 3.2593E−08 |
| LSG1 | ILMN_1248340 | 2.0393E−05 | 7.6022E−07 | 0.82592388 | 0.63417702 | 0.00012591 | 1.5794E−05 |
| RAB6 | ILMN_1242802 | 0.00019668 | 7.5901E−09 | 0.07535627 | 0.36917367 | 0.04400013 | 0.00031887 |
| ARL5A | ILMN_1221102 | 9.1695E−07 | 2.1307E−06 | 0.13976302 | 0.11920964 | 0.00069199 | 0.00618297 |
| OTTMUSG00000005523 | ILMN_2847773 | 3.7941E−06 | 7.5561E−08 | 0.51643402 | 0.81925798 | 2.0548E−05 | 4.1116E−06 |
| RN185 | ILMN_2638923 | 3.9291E−05 | 0.00064196 | 0.43982461 | 0.09151301 | 4.1387E−05 | 0.00794885 |
| A830083H19RIK | ILMN_2565089 | 3.7726E−08 | 4.6077E−07 | 0.323377 | 0.13061191 | 3.8159E−07 | 5.705E−05 |
| FYB | ILMN_1236105 | 6.7827E−06 | 3.1373E−05 | 0.4465201 | 0.88460871 | 0.00041176 | 0.00246862 |
| PUM2 | ILMN_2607160 | 1.8336E−07 | 1.0301E−10 | 0.36542615 | 0.56693674 | 7.7372E−05 | 1.2081E−06 |
| SCL0002368.1_75 | ILMN_1214650 | 0.00616283 | 0.0005403 | 0.80188852 | 0.60020322 | 4.8757E−05 | 2.2172E−05 |
| COX7A1 | ILMN_1240857 | 0.00033563 | 4.3239E−08 | 0.31458743 | 0.06118139 | 0.00565176 | 1.0553E−05 |
| NPC2 | ILMN_2744380 | 2.7328E−06 | 6.5811E−08 | 0.08296152 | 0.660283 | 0.00657288 | 0.00181981 |
| SAMHD1 | ILMN_1221736 | 1.42E−06 | 6.4508E−07 | 0.12355081 | 0.38603934 | 0.00010214 | 0.001305 |
| CLCN3 | ILMN_1221919 | 1.3015E−06 | 1.9688E−06 | 0.16718214 | 0.93533936 | 0.00079195 | 0.00298329 |
| PTPN1 | ILMN_1222917 | 3.5773E−07 | 1.0659E−05 | 0.2605158 | 0.09805243 | 0.00013636 | 0.00362761 |
| CBFB | ILMN_2671644 | 4.8743E−05 | 9.4186E−07 | 0.09254946 | 0.5793726 | 0.00990408 | 0.00230623 |
| 5830457O10RIK | ILMN_1249269 | 0.00519153 | 0.00019135 | 0.32586613 | 0.62425128 | 0.03910471 | 0.00652554 |
| HIST1H2AG | ILMN_2981801 | 0.00142037 | 4.2764E−06 | 0.50917287 | 0.18961423 | 0.01065852 | 0.00020154 |
| LOC383706 | ILMN_2535779 | 0.0002793 | 1.6795E−06 | 0.19304026 | 0.25442323 | 0.01245228 | 0.00070261 |
| TPM4 | ILMN_1223539 | 6.0659E−06 | 2.6052E−05 | 0.09185367 | 0.13528198 | 0.00233641 | 0.03571863 |
| CYB5 | ILMN_2775098 | 1.3396E−08 | 6.2407E−13 | 0.11624921 | 0.12052557 | 0.00013728 | 3.9171E−07 |
| TOP2A | ILMN_1255436 | 7.8911E−10 | 1.5724E−08 | 0.09369235 | 0.2373449 | 3.8714E−05 | 0.0009104 |
| DCUN1D5 | ILMN_1238285 | 4.1989E−06 | 1.8741E−06 | 0.38085229 | 0.37503555 | 0.00014453 | 0.00028909 |
| PPP1CB | ILMN_2647628 | 1.2809E−05 | 1.1607E−05 | 0.15887686 | 0.18184827 | 0.00454368 | 0.01745784 |
| PJA1 | ILMN_2516312 | 4.7542E−05 | 5.5089E−05 | 0.13308991 | 0.46093695 | 0.00820313 | 0.03011249 |
| CD247 | ILMN_1256639 | 9.0935E−07 | 6.4865E−10 | 0.31335383 | 0.66967268 | 0.00164992 | 3.7917E−05 |
| RNF8 | ILMN_1214110 | 0.00010135 | 2.7576E−05 | 0.13291607 | 0.87304591 | 0.01603417 | 0.01952903 |
| TRMT2A | ILMN_2682887 | 0.00340732 | 0.00028594 | 0.4813778 | 0.79961073 | 0.02046512 | 0.00569351 |
| ARL1 | ILMN_1219577 | 2.543E−06 | 5.3093E−06 | 0.06285512 | 0.25217896 | 0.00175704 | 0.01808582 |
| 4930539E08RIK | ILMN_1238069 | 3.2079E−08 | 9.7942E−09 | 0.46735317 | 0.79033315 | 4.0625E−05 | 2.1668E−05 |
| CXCR6 | ILMN_1221846 | 0.00019423 | 3.5807E−05 | 0.36711411 | 0.85413772 | 0.00840588 | 0.00451855 |
| TMPO | ILMN_2471359 | 0.00015384 | 1.421E−06 | 0.14493158 | 0.81398681 | 0.01799086 | 0.00261013 |
| IRF1 | ILMN_2649068 | 0.01825352 | 0.00874502 | 0.94796386 | 0.90430421 | 0.00483582 | 0.00588889 |
| ACTN4 | ILMN_1242390 | 0.00434152 | 0.00106316 | 0.92917511 | 0.91918226 | 0.00736922 | 0.00310551 |
| WDR45L | ILMN_1242852 | 0.00013965 | 1.0141E−05 | 0.43961482 | 0.89370272 | 0.00725724 | 0.00195908 |
| ZFP281 | ILMN_2477243 | 1.384E−06 | 4.3216E−06 | 0.31887263 | 0.18984517 | 0.00017162 | 0.00180339 |
| D6WSU163E | ILMN_1224433 | 1.1635E−07 | 2.4898E−06 | 0.07027231 | 0.16479956 | 0.00204457 | 0.02558607 |
| NUDCD2 | ILMN_2751228 | 0.00217462 | 0.00235538 | 0.48810666 | 0.50655725 | 0.0233342 | 0.0387743 |
| H2-T23 | ILMN_1223179 | 0.00482755 | 0.00049264 | 0.38544903 | 0.66476091 | 0.02352203 | 0.01043158 |
| LOC667005 | ILMN_1217929 | 0.0009143 | 0.0005654 | 0.74360086 | 0.73765156 | 0.00089637 | 0.00061251 |
| EIF3B | ILMN_1233746 | 0.00075977 | 0.00017974 | 0.33823268 | 0.86933151 | 0.01259048 | 0.00993988 |
| ELAC2 | ILMN_1255921 | 0.00132668 | 0.00012916 | 0.6032269 | 0.9503948 | 0.00866596 | 0.00232593 |
| GTL7 | ILMN_2753314 | 3.0675E−06 | 4.5499E−06 | 0.25763463 | 0.16521499 | 8.345E−05 | 0.00064529 |
| LOC100046320 | ILMN_1243283 | 0.00063385 | 0.00074976 | 0.29556725 | 0.41378961 | 0.01538515 | 0.03606488 |
| PARP14 | ILMN_2825803 | 9.76E−07 | 3.9082E−06 | 0.37203432 | 0.48900922 | 9.7791E−05 | 0.00095472 |
| PK3 | ILMN_1236328 | 5.8275E−05 | 1.9145E−06 | 0.16833572 | 0.7021492 | 0.02913291 | 0.00742425 |
| ZZZ3 | ILMN_2432040 | 0.00149654 | 5.4024E−06 | 0.37814398 | 0.23151332 | 0.04634003 | 0.00172821 |
| LOC100041450 | ILMN_1242660 | 0.00015681 | 1.4503E−06 | 0.39312239 | 0.21407306 | 0.00499112 | 0.00029809 |
| WAPAL | ILMN_1242714 | 3.611E−05 | 3.0184E−05 | 0.15289192 | 0.67266295 | 0.00959341 | 0.02197082 |
| 8030402P03RIK | ILMN_2585137 | 0.00222271 | 7.3726E−05 | 0.58637315 | 0.43338243 | 0.0221549 | 0.00251287 |
| A630098G22RIK | ILMN_2582171 | 0.00029974 | 0.00015681 | 0.1752775 | 0.42699241 | 0.02148483 | 0.03714721 |
| CSF1 | ILMN_1254561 | 1.5202E−07 | 5.7675E−06 | 0.11168983 | 0.10721403 | 0.00070307 | 0.01305309 |
| FBXO33 | ILMN_3162369 | 0.00013886 | 5.8078E−07 | 0.32151153 | 0.50625039 | 0.01673252 | 0.00095505 |
| SLCO3A1 | ILMN_1235635 | 0.00091878 | 0.00020402 | 0.73099413 | 0.98590452 | 0.00492862 | 0.00245818 |
| PDLIM1 | ILMN_1234072 | 0.00441381 | 3.9934E−05 | 0.85438667 | 0.23205806 | 0.00405604 | 7.2635E−05 |
| 2810008M24RIK | ILMN_2486600 | 0.00019745 | 8.1149E−06 | 0.35285462 | 0.83709923 | 0.00372931 | 0.0008125 |
| 1700025G04RIK | ILMN_2669982 | 7.5215E−06 | 1.3381E−05 | 0.11339445 | 0.35621273 | 0.00115884 | 0.01167827 |
| BCL11B | ILMN_2443624 | 0.00151871 | 0.00185206 | 0.07205 | 0.62583244 | 4.592E−06 | 2.4442E−06 |
| D630022O22RIK | ILMN_1223311 | 2.8672E−05 | 3.4869E−05 | 0.34002484 | 0.8660441 | 0.00303608 | 0.00610448 |
| DMBT1 | ILMN_2629239 | 2.5255E−05 | 9.8683E−06 | 0.1748778 | 0.87879249 | 0.02845532 | 0.02205192 |
| MAD2L1BP | ILMN_2815222 | 0.00121644 | 1.3699E−06 | 0.44549374 | 0.28915968 | 0.00745121 | 4.4145E−05 |
| IRF1 | ILMN_2649067 | 0.03891771 | 0.02683732 | 0.92833492 | 0.67341484 | 0.01119362 | 0.01531716 |
| ETS1 | ILMN_2770739 | 0.00142024 | 0.00354497 | 0.73817964 | 0.38828425 | 0.00409338 | 0.01680685 |
| 1810013L24RIK | ILMN_1260569 | 2.4971E−08 | 2.6034E−06 | 0.12129405 | 0.12545508 | 0.00010235 | 0.00816545 |
| TOR1A | ILMN_2631144 | 0.00079779 | 4.7212E−05 | 0.26557048 | 0.55002378 | 0.04165822 | 0.01080278 |
| TRAT1 | ILMN_1229005 | 0.00316669 | 0.00029995 | 0.61937322 | 0.72738376 | 0.00275379 | 0.00019498 |
| PABPC1 | ILMN_1259769 | 0.0022435 | 0.01046226 | 0.93750313 | 0.16988432 | 0.00195638 | 0.01372953 |
| H2AFZ | ILMN_3074985 | 0.00047158 | 0.00010411 | 0.11786485 | 0.88692979 | 0.04992566 | 0.04165536 |
| ZDHHC21 | ILMN_2780619 | 0.00667387 | 0.00033365 | 0.79075136 | 0.49723618 | 0.01966091 | 0.00240347 |
| ZNF24 | ILMN_2518828 | 0.02139095 | 0.00040401 | 0.60212275 | 0.12398231 | 0.00488301 | 0.00018603 |
| ZWILCH | ILMN_1247268 | 0.00055985 | 0.00060993 | 0.30541403 | 0.63014295 | 0.0312265 | 0.04621727 |
| AP1B1 | ILMN_1244906 | 4.3529E−05 | 2.9383E−06 | 0.12513979 | 0.80871339 | 0.02329204 | 0.01323394 |
| 2500002A22RIK | ILMN_1255902 | 9.8228E−05 | 0.00127233 | 0.74738135 | 0.31074683 | 0.00087637 | 0.01057522 |
| LOC380707 | ILMN_1252450 | 0.00124251 | 0.00177855 | 0.96350441 | 0.79424307 | 0.00145428 | 0.00344935 |
| TNP2 | ILMN_1224330 | 7.3765E−05 | 0.00016167 | 0.33030804 | 0.23292724 | 0.00444441 | 0.01925874 |
| GTF3C5 | ILMN_1222243 | 0.00595051 | 0.00011146 | 0.62796493 | 0.40463604 | 0.04041016 | 0.00293892 |
| HIST1H2AK | ILMN_2847144 | 0.00191935 | 0.00510824 | 0.88061755 | 0.40918381 | 0.00262265 | 0.00692814 |
| RNGTT | ILMN_2750669 | 0.01282445 | 0.00231609 | 0.92980705 | 0.74261519 | 0.01661577 | 0.00521343 |
| VPS37B | ILMN_2681670 | 0.00743656 | 0.00177168 | 0.27175517 | 0.93678492 | 8.9645E−05 | 1.7425E−05 |
| F830048D03RIK | ILMN_2584985 | 0.03575523 | 0.00171988 | 0.63764888 | 0.39373185 | 0.0150219 | 0.00078959 |
| USF1 | ILMN_2435505 | 0.01387975 | 0.00989901 | 0.65814913 | 0.85098378 | 0.04125975 | 0.04710474 |
| HEATR3 | ILMN_2590644 | 0.00803221 | 0.00032825 | 0.79171755 | 0.61386053 | 0.0146601 | 0.00098646 |
| STIL | ILMN_2654075 | 0.03620021 | 0.00033133 | 0.81325368 | 0.13467102 | 0.04079275 | 0.00081181 |
| FEM1C | ILMN_2724409 | 0.00034431 | 6.9389E−05 | 0.25027131 | 0.50109871 | 0.01172099 | 0.01132166 |
| EG666756 | ILMN_1214902 | 9.4507E−05 | 0.00038474 | 0.46314729 | 0.26744158 | 0.0047458 | 0.01824865 |
| PIBF1 | ILMN_3115640 | 5.5587E−05 | 0.00037181 | 0.24993336 | 0.14445566 | 0.00447915 | 0.04489547 |
| MX2 | ILMN_1239219 | 0.00686125 | 0.01088913 | 0.85986592 | 0.67417553 | 0.00478334 | 0.01069715 |
| CHIC2 | ILMN_2548974 | 0.00042825 | 0.00015318 | 0.26003101 | 0.55024362 | 0.0254452 | 0.02958371 |
| EIF4E3 | ILMN_1254878 | 6.5566E−05 | 0.00250945 | 0.62069091 | 0.07082806 | 2.8753E−06 | 0.00041643 |
| RPL37 | ILMN_1236446 | 0.00025435 | 0.00033519 | 0.28168683 | 0.5589165 | 0.01021126 | 0.02406462 |
| ELF1 | ILMN_2659380 | 0.01484891 | 0.00118441 | 0.71387567 | 0.90392318 | 0.00695924 | 0.0003911 |
| BAT2D | ILMN_1221890 | 0.00153674 | 0.00016834 | 0.83076219 | 0.57605882 | 0.01061237 | 0.00260791 |
| 4930404F20RIK | ILMN_1222969 | 0.03606049 | 0.00017185 | 0.74319886 | 0.13910106 | 0.01617178 | 7.3937E−05 |
| LOC100044190 | ILMN_1214731 | 2.7461E−05 | 0.00181642 | 0.98810296 | 0.06033325 | 0.00020412 | 0.00651301 |
| EP5TI1 | ILMN_2707865 | 0.00714158 | 0.00148007 | 0.54805412 | 0.6847784 | 0.03639476 | 0.01986918 |
| GPI1 | ILMN_2692845 | 3.3041E−06 | 0.00011199 | 0.15411737 | 0.19309211 | 0.00293692 | 0.04703852 |
| 4930422G04RIK | ILMN_1243091 | 8.401E−05 | 6.9798E−05 | 0.15352672 | 0.77267008 | 0.0350024 | 0.04869989 |
| RBM14 | ILMN_1245292 | 0.00183636 | 0.00193788 | 0.68916475 | 0.4886926 | 0.02807555 | 0.03388096 |
| 4932413O14RIK | ILMN_2620757 | 0.01270668 | 0.0007843 | 0.99548386 | 0.49086007 | 0.02165455 | 0.00245108 |
| CD247 | ILMN_2828172 | 0.00958744 | 0.00201477 | 0.45933339 | 0.79127474 | 0.00553886 | 0.0007173 |
| CCR4 | ILMN_2753620 | 0.00177622 | 0.00061868 | 0.4406932 | 0.73586934 | 0.01810352 | 0.01615626 |
| ZFP800 | ILMN_2760387 | 0.04480805 | 0.00759965 | 0.96768543 | 0.66232646 | 0.0439818 | 0.01013543 |
| PABPC1 | ILMN_1259482 | 0.0051572 | 0.01388147 | 0.65525326 | 0.32541653 | 0.00273095 | 0.00614701 |
| D230021E06RIK | ILMN_2569706 | 0.00010271 | 0.01070314 | 0.9348866 | 0.06633293 | 0.00010871 | 0.01978193 |
| SELP | ILMN_1236889 | 0.01406867 | 0.00584443 | 0.93796259 | 0.92591209 | 0.01055048 | 0.00863713 |
| TSPAN14 | ILMN_1257666 | 0.00061062 | 0.00216339 | 0.99188664 | 0.60003335 | 0.00126308 | 0.00571973 |
| V1RD18 | ILMN_2996714 | 0.00099989 | 0.0004902 | 0.37184256 | 0.6093331 | 0.03905787 | 0.03999169 |
| CD226 | ILMN_1239753 | 0.02617792 | 0.0085975 | 0.67900654 | 0.92593282 | 0.01934392 | 0.00559696 |
| Mgeneric | ILMN_2600747 | 0.00319955 | 0.00125226 | 0.58571513 | 0.92151197 | 0.01556138 | 0.01370846 |
| 2310016C16RIK | ILMN_2625854 | 3.9344E−05 | 0.00163272 | 0.43628849 | 0.21148103 | 0.00116785 | 0.03547309 |
| DCK | ILMN_1227664 | 0.00517605 | 0.01848588 | 0.33692279 | 0.39324205 | 0.00049553 | 0.00171677 |
| PCDHGC5 | ILMN_1216284 | 0.01210252 | 0.00288881 | 0.98226502 | 0.82693245 | 0.01542338 | 0.00581701 |
| UBE2Z | ILMN_2774701 | 0.00426506 | 0.00357869 | 0.89775508 | 0.80203901 | 0.022089 | 0.01972205 |
| PLXNC1 | ILMN_2756855 | 0.01321326 | 0.00998082 | 0.78883167 | 0.86528989 | 0.04381295 | 0.04278646 |
| PSEN2 | ILMN_1213684 | 0.01895622 | 0.00566864 | 0.83989169 | 0.98646115 | 0.04301241 | 0.01934652 |
| STAT3 | ILMN_2698046 | 0.00095336 | 0.01157294 | 0.81522619 | 0.3037877 | 0.00344755 | 0.03930058 |
| LOC628101 | ILMN_1213707 | 0.00453303 | 0.01110885 | 0.64564729 | 0.49430888 | 0.00478312 | 0.0087798 |
| GREM2 | ILMN_2610565 | 0.00346819 | 0.01123909 | 0.95978367 | 0.56150385 | 0.01144908 | 0.03120142 |
| INPP4B | ILMN_2854354 | 0.00012106 | 0.00458166 | 0.83008294 | 0.21443605 | 0.00079144 | 0.02082426 |
| NEO1 | ILMN_2579266 | 0.01189531 | 0.00236594 | 0.71275327 | 0.7455244 | 0.00354704 | 0.00090115 |
| 1700027N10RIK | ILMN_2775220 | 0.00878236 | 0.00099032 | 0.9594418 | 0.72932359 | 0.00772044 | 0.0015392 |
| ZFP277 | ILMN_3123889 | 0.04468996 | 0.00408357 | 0.39928703 | 0.45621364 | 0.00600161 | 0.00041052 |
| GNL3L | ILMN_2628122 | 0.00429356 | 0.01688285 | 0.85338166 | 0.39327272 | 0.00733186 | 0.04103683 |
| LOC100047009 | ILMN_1246359 | 0.00164613 | 0.00453357 | 0.61582849 | 0.52198033 | 0.00057928 | 0.00211572 |
| PPP2R5C | ILMN_3121851 | 0.01210035 | 0.02598793 | 0.95302246 | 0.32515628 | 0.00682318 | 0.02551996 |
| LOC235279 | ILMN_1231838 | 0.00223763 | 0.00052051 | 0.81148295 | 0.91497115 | 0.01510458 | 0.00593565 |
| PLEKHF2 | ILMN_1237736 | 0.00350806 | 0.01090006 | 0.9737767 | 0.27106774 | 0.00144929 | 0.0102796 |
| IL1F10 | ILMN_2776687 | 0.02678693 | 0.01379049 | 0.98525412 | 0.8846595 | 0.04957327 | 0.02966918 |
| 3202002H23RIK | ILMN_1236031 | 0.012129 | 0.00929094 | 0.48343497 | 0.66619054 | 0.00157159 | 0.00099074 |
| LOC381093 | ILMN_2530674 | 0.00372577 | 0.0023124 | 0.52468466 | 0.96763521 | 0.00230893 | 0.00130578 |
| KRT25 | ILMN_1216096 | 0.01355793 | 0.01365971 | 0.87932854 | 0.63501661 | 0.04168391 | 0.04618658 |
| VMN2R81 | ILMN_2628699 | 0.02332927 | 0.00229665 | 0.93674625 | 0.54937737 | 0.03453944 | 0.00528818 |
| FERD3L | ILMN_2724181 | 0.03286624 | 0.03206173 | 0.70598361 | 0.75164834 | 0.01726187 | 0.01719805 |
| TELO2 | ILMN_2734797 | 0.0108678 | 0.04103188 | 0.55549441 | 0.40519113 | 0.00323971 | 0.01260482 |
| 9430077D24RIK | ILMN_2524699 | 0.00316978 | 0.0424885 | 0.63122432 | 0.12110196 | 0.00016075 | 0.00868908 |
| SLC30A8 | ILMN_2686369 | 0.0253593 | 0.01188287 | 0.80331462 | 0.70604595 | 0.01856982 | 0.01145844 |
| TRAPPC2 | ILMN_2438032 | 0.01282766 | 0.01676372 | 0.99034693 | 0.79056547 | 0.01686696 | 0.02889083 |
| C920025C15RIK | ILMN_1242664 | 0.02515697 | 0.04181796 | 0.12481528 | 0.5543993 | 0.00096575 | 0.00086594 |
| KLRA1 | ILMN_1228875 | 0.02104319 | 0.01529111 | 0.93569553 | 0.61116955 | 0.02579361 | 0.01955059 |
| EG386506 | ILMN_1225532 | 0.04437272 | 0.01953425 | 0.73579779 | 0.72119617 | 0.03362092 | 0.01716204 |
| LOC383306 | ILMN_1235357 | 0.00064604 | 0.02120781 | 0.95855356 | 0.08386092 | 0.00135323 | 0.04001091 |
| VMN2R89 | ILMN_3151786 | 0.03333773 | 0.04372519 | 0.84476292 | 0.56837775 | 0.03041009 | 0.03866243 |
| LOC244219 | ILMN_2525856 | 0.04252735 | 0.01750244 | 0.53751488 | 0.82500131 | 0.01480124 | 0.00572124 |
| 2610017G09RIK | ILMN_1226459 | 0.04624861 | 0.02993806 | 0.9979677 | 0.97839313 | 0.03804994 | 0.03562141 |
| LOC386161 | ILMN_1257961 | 0.01004863 | 0.01316793 | 0.8190764 | 0.81316945 | 0.03227347 | 0.04815364 |
| NCDN | ILMN_2664579 | 0.03426609 | 0.01693101 | 0.92950627 | 0.99318281 | 0.04983082 | 0.02838848 |
| SLFN9 | ILMN_2688948 | 0.03258343 | 0.02920921 | 0.83629845 | 0.65028956 | 0.03356424 | 0.04711832 |
| LOC331243 | ILMN_1253944 | 0.00105068 | 0.00696807 | 0.97390244 | 0.58001583 | 0.00537133 | 0.02036766 |
| RAB33A | ILMN_3161828 | 0.04160855 | 0.04651985 | 0.29324892 | 0.9090289 | 0.00477561 | 0.00425553 |
| DTYMK | ILMN_2485787 | 0.00312723 | 0.00303143 | 0.72728489 | 0.54586584 | 0.01398598 | 0.01946506 |
| LOC100040244 | ILMN_1260579 | 0.0088602 | 0.02095795 | 0.5320051 | 0.32021835 | 0.00502459 | 0.00671978 |
| OS9 | ILMN_1246758 | 0.00188503 | 0.02323417 | 0.95367604 | 0.08759446 | 0.00146045 | 0.02295013 |
| D630011N09RIK | ILMN_2580554 | 0.04588181 | 0.01333751 | 0.76433191 | 0.86628684 | 0.03089735 | 0.00916026 |
| A930014D08RIK | ILMN_2519658 | 0.04862709 | 0.01498476 | 0.74061877 | 0.7386258 | 0.0170917 | 0.00652842 |
| MYNN | ILMN_1213772 | 0.00589596 | 0.01126549 | 0.76960806 | 0.4883202 | 0.01670393 | 0.04081575 |
| SPNB2 | ILMN_2681429 | 0.03267873 | 0.0349451 | 0.49423396 | 0.92906641 | 0.01836448 | 0.01653158 |
| RNF19A | ILMN_2424138 | 0.0259662 | 0.04860412 | 0.53804451 | 0.25096391 | 0.00393592 | 0.00418054 |
| ARHGAP25 | ILMN_3155245 | 0.02952979 | 0.01730665 | 0.60251038 | 0.38148931 | 0.02198482 | 0.00694113 |
| LOC383433 | ILMN_2539526 | 0.00473651 | 0.0187802 | 0.91590057 | 0.64902958 | 0.02373968 | 0.04650456 |
| MIER2 | ILMN_2671319 | 0.03613669 | 0.02398398 | 0.47986764 | 0.80556556 | 0.00582487 | 0.00297359 |
| PDIA2 | ILMN_1215125 | 0.0186685 | 0.04238035 | 0.70955857 | 0.4244174 | 0.02026283 | 0.03357452 |
| LOC384990 | ILMN_1218114 | 0.01136297 | 0.03540832 | 0.83208315 | 0.45193952 | 0.003319 | 0.02169003 |
| LOC100043391 | ILMN_2740432 | 0.00959432 | 0.04663032 | 0.43800025 | 0.3030988 | 0.00112178 | 0.00605758 |
| LOC100040854 | ILMN_1217820 | 0.03261636 | 0.03232909 | 0.2727063 | 0.8371501 | 0.00291635 | 0.0500885 |
| PIGN | ILMN_2547482 | 0.03645627 | 0.02601854 | 0.55141309 | 0.68930771 | 0.01330125 | 0.00653214 |
| BAZ1B | ILMN_1246235 | 0.01404517 | 0.02132842 | 0.89259619 | 0.42310216 | 0.02518395 | 0.03234624 |
| TTLL4 | ILMN_1251995 | 0.00921362 | 0.04352022 | 0.7539064 | 0.30450795 | 0.00888912 | 0.03336755 |
| DHX32 | ILMN_2643150 | 0.00899502 | 0.04951511 | 0.90818198 | 0.19188292 | 0.00936406 | 0.04943437 |
| D4ERTD22E | ILMN_3129206 | 0.00192543 | 0.01707086 | 0.94525616 | 0.21973825 | 0.0067388 | 0.03501916 |
| LOC100048816 | ILMN_1253242 | 0.00639681 | 0.04549597 | 0.78877209 | 0.13429775 | 0.01189648 | 0.04792802 |
| GPC3 | ILMN_1223663 | 0.01242117 | 0.04025834 | 0.88584774 | 0.46797688 | 0.01128409 | 0.04270041 |
| LOC384585 | ILMN_1243863 | 0.00455984 | 0.03457727 | 0.86533679 | 0.20462142 | 0.00645194 | 0.03772185 |
| OLFR701 | ILMN_2652694 | 0.01859231 | 0.04898341 | 0.71727788 | 0.20308366 | 0.01606462 | 0.02786974 |
| FANCA | ILMN_1220189 | 0.01861672 | 0.04654276 | 0.80570207 | 0.33726393 | 0.01436508 | 0.03325718 |

DOWN IN GA AND REF ONLY

| | | | | | | | |
|---|---|---|---|---|---|---|---|
| LOC386360 | ILMN_2538531 | 0.00181273 | 1.3056E−06 | 0.24179742 | 0.0610866 | 0.04766182 | 0.00051103 |
| CLEC4E | ILMN_1216972 | 2.3876E−08 | 1.7989E−08 | 0.29133177 | 0.07208954 | 0.00010585 | 0.0003322 |
| LOC385923 | ILMN_1230140 | 0.00232668 | 9.8497E−06 | 0.8804496 | 0.35734588 | 0.00115913 | 5.7017E−06 |
| C5F3R | ILMN_2806180 | 2.7194E−06 | 2.4676E−08 | 0.19435996 | 0.76560322 | 0.0026131 | 0.00034547 |
| CD33 | ILMN_2675223 | 2.2572E−09 | 2.9811E−09 | 0.33332323 | 0.1662516 | 1.4979E−05 | 4.5287E−05 |
| CD63 | ILMN_3128907 | 9.5785E−05 | 4.5504E−06 | 0.45437477 | 0.96599518 | 0.00297745 | 0.00067862 |
| CXCR4 | ILMN_2630459 | 4.1663E−07 | 2.3331E−05 | 0.45822739 | 0.18369107 | 0.00035307 | 0.00513661 |
| AA536749 | ILMN_1219077 | 3.0901E−09 | 1.3456E−08 | 0.05492684 | 0.82199671 | 0.00023116 | 0.00109256 |
| LOC386268 | ILMN_1222988 | 0.01901637 | 8.0896E−05 | 0.68455591 | 0.20419658 | 0.03236315 | 0.00019971 |
| genericLS | ILMN_1250635 | 3.8814E−05 | 9.224E−09 | 0.20940988 | 0.18069393 | 0.04229329 | 0.00085164 |
| GAS7 | ILMN_2776850 | 2.885E−06 | 1.1438E−06 | 0.57560127 | 0.92740131 | 3.9705E−05 | 7.8795E−05 |
| KLC4 | ILMN_1251301 | 3.9151E−08 | 6.9555E−10 | 0.11394085 | 0.84620847 | 0.00191252 | 0.00057061 |
| LOC386288 | ILMN_1257784 | 0.00018847 | 2.9332E−05 | 0.10460219 | 0.96832317 | 0.04254328 | 0.04594179 |
| TMEM38B | ILMN_2795791 | 7.3219E−07 | 5.9395E−09 | 0.11760121 | 0.64020611 | 0.00382771 | 0.00044923 |
| GPR97 | ILMN_2666492 | 1.0298E−05 | 1.0795E−06 | 0.21424248 | 0.68491315 | 0.00420588 | 0.00173932 |
| 1100001G20RIK | ILMN_2651743 | 7.7433E−06 | 9.2446E−06 | 0.22896225 | 0.53871344 | 0.00090228 | 0.003214 |
| APP | ILMN_2597532 | 1.7379E−06 | 1.9127E−07 | 0.05632768 | 0.77600634 | 0.00436728 | 0.00412349 |
| 4930572J05RIK | ILMN_3065373 | 0.00456602 | 2.5099E−05 | 0.4441944 | 0.51657983 | 0.04071292 | 0.00142629 |
| TGIF1 | ILMN_1253854 | 0.00085305 | 0.0001751 | 0.50991015 | 0.83268919 | 0.01215416 | 0.00643797 |
| LOC243025 | ILMN_1252253 | 2.0739E−06 | 5.102E−08 | 0.06597363 | 0.73849896 | 0.00691368 | 0.00206398 |
| SEC16A | ILMN_1225772 | 0.00012188 | 2.5231E−08 | 0.05001367 | 0.22328973 | 0.04852839 | 0.00083133 |
| BC014795 | ILMN_2769945 | 1.4506E−06 | 1.478E−07 | 0.35494542 | 0.92186409 | 0.00060141 | 0.00020323 |
| CFB | ILMN_1230853 | 6.1942E−05 | 0.00013352 | 0.2759896 | 0.2823461 | 0.00114292 | 0.01184922 |
| TMEM63A | ILMN_2798340 | 0.00011542 | 4.0037E−06 | 0.07854127 | 0.31886993 | 0.02885688 | 0.00527756 |
| DNMT3L | ILMN_1250149 | 0.00021396 | 7.511E−08 | 0.48180823 | 0.13801656 | 0.01177344 | 8.1017E−05 |
| ACBD4 | ILMN_3150296 | 0.00022203 | 1.2757E−06 | 0.18507885 | 0.38884459 | 0.02730026 | 0.00201988 |
| ORF19 | ILMN_3158633 | 5.1394E−07 | 1.0611E−06 | 0.06272749 | 0.28478638 | 0.00249543 | 0.01548046 |
| CREG1 | ILMN_1239638 | 8.3906E−06 | 1.5062E−07 | 0.37545788 | 0.90170705 | 0.00119168 | 0.0001639 |
| IRAK3 | ILMN_1253972 | 0.00018087 | 8.6222E−05 | 0.25300401 | 0.72925351 | 0.01665379 | 0.01352798 |
| BC065397 | ILMN_2869654 | 5.8289E−06 | 2.9074E−06 | 0.05466237 | 0.80709989 | 0.01166877 | 0.01821551 |
| ARHGAP22 | ILMN_2672380 | 8.7659E−06 | 5.3864E−07 | 0.05463559 | 0.27068336 | 0.02221169 | 0.03255185 |
| GAA | ILMN_1254199 | 1.1907E−06 | 3.8671E−07 | 0.14151281 | 0.70576013 | 0.00943593 | 0.00878139 |
| LOC386117 | ILMN_1218079 | 0.00217917 | 2.8431E−05 | 0.31341623 | 0.75826076 | 0.03529147 | 0.00332384 |
| S031414D18RIK | ILMN_2615575 | 2.2017E−05 | 3.7127E−05 | 0.24787785 | 0.9464672 | 0.00974156 | 0.01414106 |
| IRF3 | ILMN_1229320 | 8.0674E−05 | 1.5322E−05 | 0.40245934 | 0.38917543 | 0.00154207 | 0.00103721 |
| IGLC2_J00595_IG_{—} | ILMN_2450155 | 8.1454E−07 | 7.2825E−07 | 0.98555987 | 0.93707531 | 5.8297E−05 | 3.3669E−05 |
| LAMBDA_CONSTANT_{—} | | | | | | | |
| 2_14 | | | | | | | |
| BRD2 | ILMN_3154409 | 0.00021378 | 1.1794E−06 | 0.09233986 | 0.62874756 | 0.04587142 | 0.00585275 |
| SRGAP2 | ILMN_1222560 | 6.0142E−09 | 4.0425E−08 | 0.05538136 | 0.10209811 | 0.00051292 | 0.00630075 |
| ZFP36L1 | ILMN_2780424 | 6.2241E−05 | 2.4506E−07 | 0.63044859 | 0.30875858 | 0.0058726 | 0.00015962 |
| MYO7A | ILMN_1251839 | 8.979E−06 | 6.8094E−06 | 0.06035726 | 0.44757884 | 0.01254915 | 0.02940651 |
| LOC382163 | ILMN_1237356 | 2.9941E−05 | 2.225E−05 | 0.06972901 | 0.30757834 | 0.00973088 | 0.03865672 |
| LOC238836 | ILMN_2527694 | 2.3899E−06 | 1.1057E−08 | 0.13215153 | 0.90529614 | 0.00214514 | 0.00019416 |
| HRSP12 | ILMN_2610706 | 0.00047897 | 0.0002885 | 0.19803657 | 0.65976628 | 0.02506249 | 0.04300314 |
| GPS2 | ILMN_2730784 | 0.00044751 | 1.5958E−06 | 0.43507479 | 041816214 | 0.00636587 | 0.00016543 |
| DLG3 | ILMN_2993836 | 0.00012475 | 9.253E−05 | 0.32562186 | 0.49309223 | 0.00131486 | 0.00415595 |
| 2810402K13RIK | ILMN_2455390 | 6.7186E−08 | 2.1923E−05 | 0.09137556 | 0.16734358 | 0.00163011 | 0.03908702 |
| SHC1 | ILMN_2957070 | 9.8671E−06 | 9.7326E−08 | 0.23663892 | 0.32993882 | 0.00455799 | 000031763 |
| GALNT10 | ILMN_2750011 | 0.0001616 | 4.0314E−07 | 0.10401598 | 0.51819834 | 0.02877232 | 0.002004 |
| IVD | ILMN_2728379 | 1.8778E−05 | 5.595E−07 | 0.09791528 | 0.53580705 | 0.01166027 | 0.00610671 |
| RING1 | ILMN_2932539 | 0.01663431 | 1.0512E−05 | 0.38029272 | 0.07141046 | 0.00232036 | 7.68E−07 |
| LOC100048105 | ILMN_2588050 | 0.00093834 | 7.7352E−08 | 0.45732595 | 0.10557183 | 0.02315411 | 5.6826E−05 |
| CLEC4B1 | ILMN_2603898 | 0.0006499 | 8.9002E−05 | 0.94435951 | 0.53125306 | 0.00090627 | 0.00013919 |
| TYROBP | ILMN_2867147 | 4.9902E−05 | 0.00081337 | 0.32419201 | 0.08239071 | 0.00092921 | 0.03726523 |
| 6230425C21RIK | ILMN_2550095 | 3.3729E−05 | 0.00116327 | 0.76744952 | 0.22348849 | 0.00010401 | 0.00195454 |
| 2810422O20RIK | ILMN_2625188 | 0.00633296 | 2.9442E−05 | 0.8078838 | 0.43902164 | 0.01516821 | 0.00017634 |
| RFFL | ILMN_1048689 | 0.00218426 | 9.0051E−05 | 0.94688198 | 0.44926206 | 0.00755097 | 0.00070131 |
| CEPT1 | ILMN_2762334 | 0.00015346 | 3.5027E−06 | 0.15126391 | 0.26301395 | 0.03160959 | 0.00446217 |
| NEURL | ILMN_2625940 | 1.9427E−05 | 2.9768E−06 | 0.07989839 | 0.51820005 | 0.03331221 | 0.0413976 |
| ZFP317 | ILMN_2481799 | 0.00040842 | 2.0466E−05 | 0.17421304 | 0.79274798 | 0.03686878 | 0.01298618 |
| SRGAP3 | ILMN_2648098 | 0.00426435 | 4.0797E−06 | 0.56745573 | 0.07122742 | 0.0457434 | 0.00043579 |
| RARG | ILMN_3132660 | 0.0002313 | 0.0001074 | 0.09611017 | 0.45644909 | 0.02491514 | 0.04816853 |
| PI4KB | ILMN_2689259 | 0.00018335 | 0.00028206 | 0.90417538 | 0.56122401 | 0.00104389 | 0.00184457 |
| GALGT1 | ILMN_1241182 | 0.00356 | 0.00022666 | 0.96380465 | 0.62431225 | 0.00456971 | 0.00057373 |
| PIK3R6 | ILMN_3160107 | 0.00075434 | 0.00095933 | 0.98018626 | 0.73529845 | 0.00190195 | 0.00317502 |
| MREG | ILMN_2976159 | 0.01151117 | 0.00121879 | 0.97052355 | 0.5417462 | 0.00767257 | 0.00149091 |
| 1200002N14RIK | ILMN_1219820 | 0.00016287 | 0.00089278 | 0.3547823 | 0.72280221 | 6.7964E−06 | 7.9454E−05 |
| 2610028H07RIK | ILMN_1256792 | 6.3456E−06 | 0.00044627 | 0.53405371 | 0.17875439 | 8.3178E−05 | 0.00823961 |
| TGFBI | ILMN_2727663 | 9.3229E−05 | 0.00506724 | 0.69685832 | 0.07915194 | 0.00018689 | 0.02386499 |
| NOS3 | ILMN_2788593 | 0.00014498 | 3.6426E−05 | 0.14285813 | 0.8181436 | 0.0230466 | 0.01665852 |
| USP11 | ILMN_2802244 | 0.00084451 | 0.00039723 | 0.54734927 | 0.57435118 | 0.004636 | 0.00658528 |
| FOS | ILMN_2750515 | 0.01415452 | 0.00059588 | 0.50543321 | 0.28725213 | 0.00125985 | 5.3042E−05 |
| LOC636818 | ILMN_2434512 | 0.00038393 | 1.1401E−05 | 0.1989409 | 0.6183528 | 0.03774769 | 0.00837987 |
| SCL0004023.1_57 | ILMN_1232512 | 0.00026729 | 3.1473E−05 | 0.71431891 | 0.74464801 | 0.00278043 | 0.00081738 |
| P2RX4 | ILMN_2620122 | 0.00081634 | 0.00083088 | 0.37867114 | 0.71855317 | 0.01190731 | 0.03061122 |
| DGKQ | ILMN_2972541 | 0.00048918 | 0.00052041 | 0.26951773 | 0.52599589 | 0.021019 | 0.04778715 |
| SNX15 | ILMN_2894450 | 0.00937026 | 4.6424E−05 | 0.24248379 | 0.08593987 | 0.00016008 | 6.0629E−07 |
| SLC11A1 | ILMN_2692797 | 0.00396693 | 0.00197835 | 0.26220177 | 0.83242628 | 0.00024944 | 5.8292E−05 |
| TLR13 | ILMN_2735961 | 2.3186E−06 | 5.5483E−05 | 0.23133935 | 0.05270935 | 0.00140451 | 0.02725246 |
| MKL1 | ILMN_2630120 | 0.0001341 | 0.00013031 | 0.2256236 | 0.65497127 | 0.01485918 | 0.02734326 |
| EG633640 | ILMN_2977390 | 0.00216317 | 1.0488E−05 | 0.37060957 | 0.13026635 | 0.02334582 | 0.00084582 |
| RASGRF1 | ILMN_1233146 | 6.1989E−05 | 1.7706E−05 | 0.18251154 | 0.69726381 | 0.04139695 | 0.03867404 |
| TRP53 | ILMN_2466845 | 0.0016928 | 0.00286906 | 0.99232057 | 0.79140758 | 0.01079811 | 0.01310186 |
| 1300010F03R1K | ILMN_1249480 | 0.00209035 | 0.00024475 | 0.95901568 | 0.59454853 | 0.00174019 | 0.00055466 |
| RNF123 | ILMN_2975900 | 0.00197116 | 0.00673377 | 0.68791146 | 0.54822315 | 0.00955467 | 0.04052037 |
| LOC386085 | ILMN_1258858 | 0.00703699 | 0.00244792 | 0.99310015 | 0.67180078 | 0.02342162 | 0.00991139 |
| TIMM8A2 | ILMN_2983631 | 0.00047977 | 7.984E−05 | 0.26017033 | 0.9288078 | 0.0408017 | 0.02577468 |
| CLEC4A1 | ILMN_1230708 | 6.3099E−05 | 0.00068046 | 0.07959173 | 0.13896852 | 6.2919E−07 | 1.1215E−06 |
| SGPL1 | ILMN_2936380 | 0.00442525 | 0.00431832 | 0.24420322 | 0.83977453 | 1.1989E−05 | 5.4288E−05 |
| TMUB1 | ILMN_2932660 | 3.6767E−06 | 2.8669E−06 | 0.55854301 | 0.18294726 | 9.6006E−05 | 0.00016686 |
| LOC383099 | ILMN_1230327 | 1.0292E−05 | 3.4316E−05 | 0.19353408 | 0.35776115 | 0.00264846 | 0.01315147 |
| HMOX1 | ILMN_2788073 | 0.00144202 | 0.01360724 | 0.52592266 | 0.11915799 | 0.00012857 | 0.00150214 |
| ERCC4 | ILMN_1259904 | 2.9635E−05 | 7.1893E−06 | 0.3072808 | 0.39378835 | 0.00204008 | 0.00190429 |
| SOLH | ILMN_2508416 | 4.9529E−05 | 0.00037941 | 0.39198068 | 0.1710492 | 0.00514359 | 0.0319132 |
| PREP | ILMN_2549494 | 8.777E−06 | 7.1078E−06 | 0.0728319 | 0.94699085 | 0.03196963 | 0.03975814 |
| LOC100048105 | ILMN_1220172 | 0.00525336 | 5.0233E−06 | 0.65150836 | 0.10450442 | 0.03793765 | 0.00030515 |
| GSTM2 | ILMN_1251449 | 1.0736E−06 | 0.00042941 | 0.62871915 | 0.0626658 | 0.000349 | 0.01622081 |
| EPB4.1L3 | ILMN_1244272 | 0.00042364 | 0.00026884 | 0.97622105 | 0.75352508 | 0.00234329 | 0.00159748 |
| USP22 | ILMN_2990220 | 0.00047972 | 7.2178E−05 | 0.6311193 | 0.73294117 | 0.00010901 | 2.9959E−05 |
| 1700073E17RIK | ILMN_2790902 | 0.0009735 | 0.00100707 | 0.33186085 | 0.79236551 | 0.02728985 | 0.04433204 |
| 1110014N23RIK | ILMN_2737979 | 0.00032186 | 6.9718E−05 | 0.24350554 | 0.9731238 | 0.01647809 | 0.01029025 |
| RCCD1 | ILMN_2838629 | 0.00854494 | 0.0014522 | 0.9523493 | 0.72271895 | 0.00298628 | 0.00110827 |
| ANKRD13A | ILMN_2613696 | 0.00484825 | 0.00169963 | 0.63330234 | 0.63720453 | 0.01182324 | 0.01176163 |
| SP2 | ILMN_2464693 | 0.00076915 | 0.00052171 | 0.61547888 | 0.86375881 | 0.02190281 | 0.01541189 |
| CENTG3 | ILMN_2754596 | 0.00085388 | 0.00664359 | 0.76336296 | 0.26483929 | 0.0048462 | 0.03241858 |
| 3110004L20RIK | ILMN_3142391 | 0.00493059 | 0.00089782 | 0.8322505 | 0.86075865 | 0.00118759 | 0.00038595 |
| 6430526N21RIK | ILMN_2556989 | 3.6563E−06 | 0.0030831 | 0.80628337 | 0.05397035 | 0.00012243 | 0.02050435 |
| 2310067B10RIK | ILMN_1245489 | 0.00010947 | 0.00212062 | 0.70454808 | 0.14966703 | 0.00188647 | 0.0207657 |
| 1810030N24RIK | ILMN_1246756 | 0.00033142 | 0.0001049 | 0.25777434 | 0.77317879 | 0.02651017 | 0.02535251 |
| RBM15 | ILMN_2945275 | 0.00020165 | 1.1186E−05 | 0.76256535 | 0.62608556 | 0.00114438 | 0.00015067 |
| WWP2 | ILMN_1226970 | 0.00131633 | 1.9694E−05 | 0.51815406 | 0.17548469 | 0.0010361 | 2.5328E−05 |
| 2610024M03RIK | ILMN_1255611 | 0.00075119 | 1.9877E−05 | 0.27695778 | 0.62454234 | 0.03720414 | 0.0073064 |
| C430003P19RIK | ILMN_2864393 | 0.02500932 | 0.00010247 | 0.73622573 | 0.12973571 | 0.03837532 | 0.0003539 |
| BC066107 | ILMN_2903889 | 0.00138848 | 0.00115874 | 0.52290823 | 0.66118654 | 0.00022804 | 0.00020897 |
| 3110082D06RIK | ILMN_2512770 | 0.00081263 | 0.00024591 | 0.41914414 | 0.87896906 | 0.01838394 | 0.01157934 |
| LYNX1 | ILMN_1256369 | 0.00420568 | 0.00073274 | 0.49989346 | 0.74691395 | 0.02527485 | 0.01173162 |
| PRKCZ | ILMN_1221045 | 0.00086771 | 0.00805577 | 0.81684994 | 0.14017939 | 0.00011414 | 0.00316194 |
| IGK-V33 | ILMN_1248308 | 0.01900936 | 0.04300612 | 0.83572809 | 0.70534644 | 0.00693678 | 0.03160553 |
| SLC11A1 | ILMN_2674884 | 0.00451734 | 0.03091521 | 0.12502748 | 0.13605457 | 1.7865E−05 | 6.4065E−05 |
| CBLC | ILMN_2738699 | 0.00012903 | 0.00348477 | 0.53465768 | 0.1576133 | 0.00041268 | 0.02925041 |
| B930049N04RIK | ILMN_2567282 | 0.00828515 | 0.00041872 | 0.71325464 | 0.62136789 | 0.03141276 | 0.00412999 |
| CASP1 | ILMN_1247592 | 0.03654543 | 0.00027824 | 0.98550996 | 0.15134683 | 0.03489466 | 0.00041029 |
| MBD1 | ILMN_2893081 | 0.00069041 | 0.00098846 | 0.93417844 | 0.49518718 | 0.00116956 | 0.00263867 |
| 2510022D24RIK | ILMN_2431257 | 0.00054235 | 0.00054186 | 0.66212847 | 0.85551939 | 0.00963869 | 0.01053503 |
| FRMPD1 | ILMN_2625610 | 0.00784766 | 8.5488E−05 | 0.68969527 | 0.17557898 | 0.04875253 | 0.00220945 |
| USP54 | ILMN_2418324 | 0.00955473 | 0.00040156 | 0.98851966 | 0.3747777 | 0.00989274 | 0.00081425 |
| FA2H | ILMN_2746783 | 0.00471864 | 0.00063209 | 0.45770422 | 0.98284131 | 0.03293531 | 0.01311008 |
| EG434402 | ILMN_2546510 | 0.00012823 | 5.5232E−05 | 0.13563773 | 0.2699795 | 0.00957843 | 0.01855157 |
| SLC9A4 | ILMN_2732246 | 0.00212409 | 0.00012482 | 0.38428076 | 0.89045568 | 0.02745037 | 0.00681249 |
| ATP6V1B2 | ILMN_2680440 | 0.03317358 | 0.00264861 | 0.22712659 | 0.23499064 | 0.00205645 | 0.00015479 |
| 2310079N02RIK | ILMN_2635801 | 0.00127502 | 7.9145E−05 | 0.60004293 | 0.90855532 | 0.01442916 | 0.00298576 |
| ZFPL1 | ILMN_1217262 | 0.01031094 | 0.01048771 | 0.44196462 | 0.87815982 | 0.00112726 | 0.00145406 |
| NISCH | ILMN_1237959 | 0.00048874 | 0.00222847 | 0.99479624 | 0.13624642 | 0.00139578 | 0.00540912 |
| FCMD | ILMN_2514924 | 0.00842949 | 0.00389628 | 0.94954546 | 0.62918162 | 0.0175612 | 0.01017213 |
| 1810009N02RIK | ILMN_2764607 | 0.01347776 | 0.00127083 | 0.13924507 | 0.56903812 | 0.00021877 | 7.5976E−06 |
| 6720486H14RIK | ILMN_1232302 | 0.01362645 | 0.00042467 | 0.86679528 | 0.54436584 | 0.04220789 | 0.00336882 |
| AW011738 | ILMN_1248890 | 0.02108937 | 0.00240422 | 0.7444499 | 0.25784546 | 0.04664972 | 0.01260325 |
| COgeneric | ILMN_2596412 | 0.00282433 | 0.00222355 | 0.6376439 | 0.47806652 | 0.01544423 | 0.01993856 |
| PCBP2 | ILMN_2599777 | 0.00077848 | 0.00010227 | 0.31573395 | 0.74194527 | 0.01637766 | 0.00940386 |
| C330019G07RIK | ILMN_1256807 | 0.01890697 | 0.00450636 | 0.95648663 | 0.87352749 | 0.01770155 | 0.00587318 |
| SMARCE1 | ILMN_2880246 | 0.00235369 | 0.00147137 | 0.52242213 | 0.4944281 | 0.02542484 | 0.03273696 |
| ABCC4 | ILMN_2621708 | 0.00334186 | 0.00235984 | 0.86637873 | 0.882039 | 0.00962734 | 0.00964223 |
| GTPBP1 | ILMN_2510196 | 0.00949724 | 0.01637229 | 0.86230464 | 0.53566936 | 0.00830264 | 0.01603778 |
| SLC25A42 | ILMN_2810937 | 0.0030623 | 0.00384662 | 0.86265789 | 0.50922403 | 0.00769803 | 0.00804271 |
| 5830415F09RIK | ILMN_1223695 | 1.3138E−05 | 0.00043694 | 0.26584773 | 0.15351078 | 0.00205106 | 0.04059407 |
| PEO1 | ILMN_2913845 | 0.00020698 | 0.00377613 | 0.64758251 | 0.27928463 | 0.00324238 | 0.03643575 |
| CRTAC1 | ILMN_2764112 | 0.03144975 | 0.00505976 | 0.48157758 | 0.34439961 | 0.00865001 | 0.00180081 |
| CLEC5A | ILMN_3045984 | 4.5951E−05 | 0.00236436 | 0.99526109 | 0.06516797 | 0.00013986 | 0.00584053 |
| ZFP704 | ILMN_1225146 | 0.01579789 | 0.00229856 | 0.43616799 | 0.40602963 | 0.0051091 | 0.00081793 |
| TNS4 | ILMN_1236029 | 0.04473469 | 0.00352156 | 0.61562326 | 0.40661935 | 0.03355411 | 0.0029568 |
| 2310040G07RIK | ILMN_1232245 | 0.00146763 | 0.00364402 | 0.9801709 | 0.53388414 | 0.00181963 | 0.00649554 |
| KRTAP5-4 | ILMN_2817498 | 0.01183825 | 0.0033168 | 0.78482862 | 0.60473247 | 0.03649514 | 0.01754729 |
| D030051N19RIK | ILMN_2760593 | 0.01767242 | 0.00883238 | 0.76269636 | 0.95269059 | 0.00352646 | 0.00288209 |
| A430043M19RIK | ILMN_2553758 | 0.01537231 | 0.00153703 | 0.87017812 | 0.70000022 | 0.02160036 | 0.00379348 |
| 9030025P20RIK | ILMN_2716783 | 0.02550031 | 0.00104391 | 0.91013697 | 0.5226017 | 0.02473964 | 0.00134412 |
| GAS7 | ILMN_2924831 | 0.00028142 | 0.01557949 | 0.48907458 | 0.14508923 | 7.41E−05 | 0.00413119 |
| TBC1D25 | ILMN_2722583 | 0.00017407 | 0.00060187 | 0.75083046 | 0.17257444 | 0.00034842 | 0.00076554 |
| LOC193563 | ILMN_1234524 | 0.02030144 | 0.00211946 | 0.49373358 | 0.37536915 | 0.00983473 | 0.0011714 |
| PAPSS2 | ILMN_2466809 | 0.00071141 | 0.01063233 | 0.59693082 | 0.1975621 | 0.00013054 | 0.00289354 |
| ELL | ILMN_2751304 | 0.00438741 | 0.00218513 | 0.56711519 | 0.94783093 | 0.03459302 | 0.0297732 |
| SHKBP1 | ILMN_2734334 | 0.00530952 | 0.04351038 | 0.45812368 | 0.22916899 | 0.00016389 | 0.00459412 |
| RNF10 | ILMN_2789888 | 0.00279888 | 0.00046885 | 0.95665835 | 0.91048263 | 0.00981439 | 0.00260664 |
| FBXL20 | ILMN_2729097 | 0.02354414 | 0.00902699 | 0.98436722 | 0.99571993 | 0.03787074 | 0.01902513 |
| TEAD4 | ILMN_3157399 | 0.01416372 | 0.00278621 | 0.95542857 | 0.71344982 | 0.04664746 | 0.01378693 |
| 5031439G07RIK | ILMN_1252809 | 0.03621453 | 0.00540906 | 0.75844219 | 0.7313907 | 0.02073207 | 0.0032499 |
| LOC100046003 | ILMN_2427178 | 0.00182928 | 0.00129669 | 0.7160404 | 0.98258458 | 0.03364079 | 0.02435778 |
| B130002B06RIK | ILMN_1231100 | 0.002085 | 0.00090773 | 0.62757755 | 0.91550262 | 0.01276374 | 0.01060191 |
| EPN3 | ILMN_1239908 | 0.00812622 | 0.00185569 | 0.60868186 | 0.71301205 | 0.04085733 | 0.02008862 |
| ZC3H12A | ILMN_2856095 | 0.02389199 | 0.01529772 | 0.10411233 | 0.95791165 | 0.0012163 | 0.00033043 |
| 2900079J23RIK | ILMN_2458967 | 0.00069102 | 0.00591699 | 0.8841941 | 0.25987594 | 0.00345581 | 0.02181717 |
| CLEC4B1 | ILMN_2959372 | 0.00498803 | 0.00566592 | 0.11687934 | 0.73202329 | 9.3941E−05 | 5.1484E−05 |
| 1300010F03RIK | ILMN_2688415 | 0.01113164 | 0.00445109 | 0.62501012 | 0.90306096 | 0.04178098 | 0.03179851 |
| KRT39 | ILMN_2699061 | 0.00024569 | 0.00320691 | 0.66536592 | 0.16397925 | 0.0044085 | 0.03601225 |
| 4930519F16RIK | ILMN_2812960 | 0.01504358 | 0.01195164 | 0.71174978 | 0.86229548 | 0.01286921 | 0.0100083 |
| RHBDD1 | ILMN_2680086 | 0.00195624 | 0.02653141 | 0.7428231 | 0.15361303 | 0.00049567 | 0.01251011 |
| GDPD1 | ILMN_2683528 | 0.02520471 | 0.01422595 | 0.97421843 | 0.97690298 | 0.02916103 | 0.02183014 |
| ACP2 | ILMN_2513205 | 0.02589288 | 0.04801525 | 0.87836357 | 0.51944157 | 0.00908429 | 0.02984164 |
| SETD1A | ILMN_1247099 | 0.02914101 | 0.01802935 | 0.28928564 | 0.80168731 | 0.00238613 | 0.0009068 |
| OLFR516 | ILMN_2975511 | 0.00584534 | 0.0065686 | 0.63189054 | 0.84803924 | 0.00463061 | 0.00502368 |
| REN1 | ILMN_2620114 | 0.00859008 | 0.00352545 | 0.51945187 | 0.91893522 | 0.04593342 | 0.03987798 |
| 5330417C22RIK | ILMN_3160472 | 0.00392981 | 0.01665235 | 0.81830592 | 0.32937105 | 0.01239845 | 0.02860146 |
| AGAP1 | ILMN_3086136 | 0.00130122 | 0.01428369 | 0.21779185 | 0.14376273 | 0.00028029 | 0.00083298 |
| DUSP6 | ILMN_2701321 | 0.04196105 | 0.02077271 | 0.65641691 | 0.76670145 | 0.01513408 | 0.00532881 |
| CTSL | ILMN_2904703 | 0.00823647 | 0.03369871 | 0.68853105 | 0.39471797 | 0.00332489 | 0.01721775 |
| SLCO6C1 | ILMN_2672845 | 0.00366169 | 0.00342522 | 0.82980707 | 0.5075665 | 0.00935595 | 0.00734696 |
| TBC1D2 | ILMN_3140913 | 0.00915868 | 0.02707865 | 0.07761914 | 0.11615683 | 5.3058E−05 | 8.3442E−06 |
| OPHN1 | ILMN_1218910 | 0.00705623 | 0.01676055 | 0.9612701 | 0.52677431 | 0.0127471 | 0.02924277 |
| B930048N13RIK | ILMN_1228496 | 0.0258409 | 0.02319307 | 0.9459577 | 0.68607919 | 0.03246647 | 0.03648165 |
| MBL1 | ILMN_1238266 | 0.01949167 | 0.01875506 | 0.6216545 | 0.95383414 | 0.0038567 | 0.00703776 |
| A730017D01RIK | ILMN_2448591 | 0.03514439 | 0.02508309 | 0.27718742 | 0.97892995 | 0.00287396 | 0.00217489 |
| SNX33 | ILMN_1212693 | 0.00128354 | 0.03546496 | 0.75931824 | 0.07681982 | 0.00178637 | 0.03039622 |
| E330023P07RIK | ILMN_1240762 | 0.04039212 | 0.01005134 | 0.90042083 | 0.69958318 | 0.04258642 | 0.01766006 |
| 9330152L17 | ILMN_2726288 | 0.00684962 | 0.00996086 | 0.82186542 | 0.65140859 | 0.003115 | 0.00702013 |
| PCDH19 | ILMN_2696829 | 0.00978932 | 0.01311307 | 0.97263701 | 0.97027419 | 0.01574332 | 0.02508658 |
| CAGE1 | ILMN_2861743 | 0.00027029 | 0.0145488 | 0.78092466 | 0.06716972 | 0.00063126 | 0.01592406 |
| TMEM56 | ILMN_2966162 | 0.04621081 | 0.01848064 | 0.41053319 | 0.64039374 | 0.00912994 | 0.00384627 |
| ACTR3B | ILMN_2933346 | 0.02450319 | 0.02824142 | 0.89846354 | 0.89324469 | 0.01546771 | 0.02801535 |
| ADAM11 | ILMN_2629383 | 0.00262479 | 0.00955767 | 0.96020583 | 0.32052216 | 0.01364716 | 0.03389781 |
| CD151 | ILMN_2836710 | 0.0193575 | 0.02521425 | 0.95832059 | 0.57385692 | 0.027328 | 0.04202571 |
| A930014P08RIK | ILMN_1258114 | 0.01653191 | 0.02263524 | 0.79892253 | 0.95914953 | 0.02026464 | 0.02963791 |
| SLC35E4 | ILMN_1225229 | 0.01200013 | 0.03647823 | 0.21422706 | 0.58229293 | 0.00060842 | 0.00210222 |
| PARD3B | ILMN_2860656 | 0.02213623 | 0.02424764 | 0.78712766 | 0.35731602 | 0.01804729 | 0.01466449 |
| OLFR586 | ILMN_2600115 | 0.02629307 | 0.03005413 | 0.44544663 | 0.51258989 | 0.01126709 | 0.00747311 |
| D030072B18RIK | ILMN_2611773 | 0.0121881 | 0.01382966 | 0.70420004 | 0.55203728 | 0.00481466 | 0.00551134 |
| STARD3 | ILMN_2769734 | 0.03250946 | 0.04372938 | 0.71591427 | 0.61539387 | 0.02634594 | 0.02932626 |
| SFXN3 | ILMN_1216734 | 0.0084465 | 0.0369416 | 0.33324991 | 0.43156506 | 0.00046311 | 0.00271253 |
| 6430511F03 | ILMN_2420209 | 0.01059293 | 0.02963372 | 0.59561896 | 0.68298302 | 0.00511949 | 0.01419095 |
| PROM | ILMN_1238150 | 0.00398623 | 0.00815465 | 0.46628964 | 0.43952028 | 0.00060491 | 0.00106285 |
| NTE | ILMN_2777668 | 0.00220455 | 0.02337669 | 0.82551283 | 0.1273946 | 0.00093989 | 0.01435096 |
| OLFR150 | ILMN_2541702 | 0.02058614 | 0.00863685 | 0.8151015 | 0.77184185 | 0.03584112 | 0.02222814 |
| 2010001M09RIK | ILMN_2613601 | 0.01200127 | 0.02267285 | 0.05308598 | 0.38262456 | 1.2109E−05 | 6.3313E−06 |
| KCNC4 | ILMN_2771095 | 0.01571387 | 0.04231651 | 0.52445469 | 0.4022856 | 0.00077199 | 0.00451424 |
| GM498 | ILMN_1247100 | 0.04366672 | 0.00882859 | 0.49722158 | 0.70284119 | 0.02936346 | 0.00526776 |
| LOC327767 | ILMN_2533815 | 0.00758783 | 0.03702409 | 0.37817812 | 0.30514982 | 0.00251604 | 0.00697011 |
| B930097H17RIK | ILMN_2573844 | 0.00260747 | 0.02509722 | 0.45261455 | 0.1224496 | 0.00074893 | 0.00496039 |
| BMP3 | ILMN_1235433 | 0.02468219 | 0.04851172 | 0.54373792 | 0.92906248 | 0.01978256 | 0.02850556 |
| PLEKHB2 | ILMN_2731769 | 0.00137797 | 0.02875588 | 0.93502443 | 0.09916793 | 0.00137931 | 0.04515583 |
| A630038P11RIK | ILMN_1233452 | 0.01134274 | 0.04302029 | 0.69787853 | 0.50924822 | 0.00488697 | 0.02393866 |
| 6720480F11RIK | ILMN_1255586 | 0.03422994 | 0.03724735 | 0.83913074 | 0.56351111 | 0.04057142 | 0.03869267 |
| CNTNAP3 | ILMN_1219653 | 0.03377393 | 0.04485233 | 0.20111585 | 0.63592426 | 0.00304714 | 0.00211903 |

| | | AVG | | | | | | |
|---|---|---|---|---|---|---|---|---|
| | AVG | Reference | | | | | | |
| Gene | Medium | Standard | AVG GA | AVG generic | | | | |
| DOWN IN generic ONLY | | | | | | | | |
| SATB1 | 8.554353333 | 8.466363158 | 8.510626471 | 8.184818182 | | | | |
| ANKRD37 | 7.88428 | 7.8945 | 7.976713235 | 7.532354545 | | | | |
| PDX1 | 7.99527 | 7.908131579 | 7.945248529 | 7.658463636 | | | | |
| A830055I09RIK | 9.037106667 | 8.940439474 | 8.998422059 | 8.727586364 | Pattern Represented by Data: | | | |
| CD2 | 10.80060333 | 10.76561316 | 10.76611912 | 10.51960455 | | | | |
| IFIT3 | 10.83471 | 10.87340526 | 10.82387647 | 10.55753636 | x | x | x | |
| TMSB10 | 11.75164 | 11.70039474 | 11.75035 | 11.47584545 | | | | x |
| PELI1 | 8.998133333 | 8.992394737 | 8.946526471 | 8.738936364 | Medium | Reference | GA | generic |
| LOC665425 | 8.158676667 | 8.102307895 | 8.112635294 | 7.902613636 | | | | |
| 5830468F06RIK | 7.97065 | 7.928986842 | 7.923582353 | 7.718622727 | | | | |
| BCL11B | 10.96429667 | 10.98868158 | 11.02490882 | 10.71570909 | | | | |
| SAMHD1 | 10.10535667 | 10.01243684 | 10.04589412 | 9.859695455 | | | | |
| IGFBP4 | 9.80274 | 9.791734211 | 9.751577941 | 9.567136364 | | | | |
| OTTMUSG00000007855 | 8.43207 | 8.3223 | 8.323457353 | 8.198613636 | | | | |
| A530024C08RIK | 7.640853333 | 7.602289474 | 7.590460294 | 7.425509091 | | | | |
| D330001F19RIK | 7.24253 | 7.214571053 | 7.194076471 | 7.031618182 | | | | |
| C230075M21RIK | 9.102993333 | 9.175807895 | 9.128044118 | 8.894036364 | | | | |
| TERF2IP | 9.187036667 | 9.121160526 | 9.125073529 | 8.978186364 | | | | |
| ARID5B | 7.844363333 | 7.88215 | 7.823404412 | 7.636695455 | | | | |
| VPS13A | 7.34862 | 7.289765789 | 7.273944118 | 7.143786364 | | | | |
| PPM1B | 6.483146667 | 6.413578947 | 6.438429412 | 6.27875 | | | | |
| ZFP281 | 9.17416 | 9.124147368 | 9.134429412 | 8.976263636 | | | | |
| 2610027C15RIK | 7.341113333 | 7.286360526 | 7.281451471 | 7.143454545 | | | | |
| BC031575 | 6.823543333 | 6.739026316 | 6.751310294 | 6.626427273 | | | | |
| PELI1 | 9.126876667 | 9.144215789 | 9.146523529 | 8.930227273 | | | | |
| RNF125 | 6.7446 | 6.727952632 | 6.689019118 | 6.548077273 | | | | |
| 1500005K14RIK | 6.293223333 | 6.218907895 | 6.253997059 | 6.102204545 | | | | |
| 1110059E24RIK | 10.46984 | 10.40856579 | 10.44254265 | 10.27383636 | | | | |
| GSN | 6.616233333 | 6.532494737 | 6.545022059 | 6.421909091 | | | | |
| ARL6IP5 | 10.5927 | 10.53820789 | 10.56679853 | 10.39869545 | | | | |
| NOTCH1 | 8.46399 | 8.526157895 | 8.466470588 | 8.270063636 | | | | |
| 9130422G05RIK | 8.094413333 | 8.017089474 | 8.024388235 | 7.903022727 | | | | |
| LOC333818 | 6.493086667 | 6.426 | 6.434104412 | 6.302809091 | | | | |
| PTPRC | 7.315573333 | 7.284710526 | 7.254329412 | 7.125622727 | | | | |
| KMO | 7.087556667 | 7.038618421 | 7.02755 | 6.898859091 | | | | |
| WIPF1 | 10.24916333 | 10.22402105 | 10.21050441 | 10.05097727 | | | | |
| FOXK1 | 7.912773333 | 7.866897368 | 7.879917647 | 7.726354545 | | | | |
| 2310032M22RIK | 7.250013333 | 7.273057895 | 7.21885 | 7.0662 | | | | |
| RPS25 | 11.92878333 | 12.00342368 | 11.90966324 | 11.74598636 | | | | |
| LOC665446 | 8.40817 | 8.473381579 | 8.482916176 | 8.2254 | | | | |
| LOC237512 | 12.71109333 | 12.65293947 | 12.65114265 | 12.53201364 | | | | |
| LOC386068 | 6.033483333 | 5.962060526 | 6.008877941 | 5.856231816 | | | | |
| ZFP429 | 7.935246667 | 7.8677 | 7.885716176 | 7.758968182 | | | | |
| BCL2L11 | 7.933673333 | 7.893531579 | 7.905207353 | 7.758081818 | | | | |
| CUGBP2 | 8.723653333 | 8.694839474 | 8.636048529 | 8.528254545 | | | | |
| SLPN1 | 10.68426 | 10.72121053 | 10.70865294 | 10.59982727 | | | | |
| KLHDC1 | 7.695726667 | 7.686955263 | 7.622123529 | 7.521354545 | | | | |
| CRAMP1L | 8.978753333 | 8.936526316 | 8.937939706 | 8.605404545 | | | | |
| CTSE | 9.17455 | 9.174597368 | 9.144792647 | 9.003390909 | | | | |
| CGGBP1 | 10.34921667 | 10.33062895 | 10.34524412 | 10.17924545 | | | | |
| A130075K23RIK | 7.857343333 | 7.866847368 | 7.817598529 | 7.687945455 | | | | |
| CDKN1B | 5.826616667 | 5.781607895 | 5.784983824 | 5.658118182 | | | | |
| HLTF | 6.093073333 | 6.029134211 | 6.027913235 | 5.924654545 | | | | |
| FNBP4 | 7.02603 | 7.015605263 | 6.975920588 | 6.858595455 | | | | |
| LOC623121 | 7.308336667 | 7.396323684 | 7.352626471 | 7.142136364 | | | | |
| 3300001P08RIK | 10.70506 | 10.66184211 | 10.65289118 | 10.53989545 | | | | |
| LOC100048622 | 9.917543333 | 9.924692105 | 9.894410294 | 9.753277273 | | | | |
| C430005L07RIK | 6.22569 | 6.156028947 | 6.162217647 | 6.062545455 | | | | |
| ZFP654 | 7.83237 | 7.779128947 | 7.811010294 | 7.670627273 | | | | |
| E030040J04RIK | 6.016863333 | 5.998997368 | 6.014368235 | 5.85555 | | | | |
| ARL4C | 8.98283 | 8.969447368 | 9.037364706 | 8.822018182 | | | | |
| ARID4A | 7.24665 | 7.221260526 | 7.191375 | 7.086068182 | | | | |
| LOC546629 | 6.201816667 | 6.155281579 | 6.149257353 | 6.041940909 | | | | |
| TDP1 | 7.001043333 | 6.9467 | 6.956392647 | 6.841454545 | | | | |
| CUL1 | 8.832946667 | 8.763292105 | 8.772133824 | 8.675431818 | | | | |
| D030041G07RIK | 7.062546667 | 7.013210526 | 6.998004412 | 6.907159091 | | | | |
| E130014H08RIK | 6.120063333 | 6.056794737 | 6.057810294 | 5.964904545 | | | | |
| TRAF5 | 6.81995 | 6.790965789 | 6.768398529 | 6.664904545 | | | | |
| PPP2R5C | 6.077013333 | 6.021755263 | 6.024205882 | 5.922486364 | | | | |
| MTF2 | 10.53759667 | 10.48152632 | 10.49541324 | 10.38590909 | | | | |
| WDR20B | 5.85008 | 5.813589474 | 5.830157353 | 5.698718182 | | | | |
| RNF125 | 7.012443333 | 7.011668421 | 7.063036765 | 6.861245455 | | | | |
| 9330168G06RIK | 6.250186667 | 6.227921053 | 6.225692647 | 6.100836364 | | | | |
| LAMP2 | 6.187146667 | 6.178439474 | 6.124277941 | 6.037936364 | | | | |
| JMJD1A | 8.215583333 | 8.183889474 | 8.228458824 | 8.066877273 | | | | |
| ST8SIA4 | 9.773773333 | 9.715015789 | 9.749726471 | 9.625213636 | | | | |
| LRBA | 9.108856667 | 9.059176316 | 9.061001471 | 8.960695455 | | | | |
| TDE2 | 9.271143333 | 9.2688 | 9.211947059 | 9.123181818 | | | | |
| 9/6/2013 | 7.035323333 | 7.010981579 | 6.982983824 | 6.888654545 | | | | |
| D830015B12RIK | 6.263736667 | 6.227584211 | 6.208301471 | 6.117745455 | | | | |
| PARD6G | 7.15756 | 7.144507895 | 7.145636765 | 7.011668182 | | | | |
| 4933400A11RIK | 5.711286667 | 5.661910526 | 5.690280882 | 5.565481818 | | | | |
| 2210016L21RIK | 5.983086667 | 5.929236842 | 5.940947059 | 5.837568182 | | | | |
| MDM1 | 5.990166667 | 5.953171053 | 5.957473529 | 5.8456 | | | | |
| CDC37L1 | 6.163896667 | 6.133318421 | 6.153929412 | 6.019554545 | | | | |
| B230215E14RIK | 6.061116667 | 6.044002632 | 6.024869118 | 5.917009091 | | | | |
| POM121 | 7.310896667 | 7.342360526 | 7.30295 | 7.167040909 | | | | |
| FOXN3 | 8.376153333 | 8.329744737 | 8.333529412 | 8.232445455 | | | | |
| A130021K16RIK | 5.984683333 | 5.9924 | 5.964948529 | 5.84155 | | | | |
| DAG1 | 7.180286667 | 7.147947368 | 7.115838235 | 7.037163636 | | | | |
| BIRC6 | 6.940266667 | 6.915202632 | 6.908766176 | 6.797263636 | | | | |
| 4930529C04RIK | 6.085303333 | 6.024965789 | 6.048104412 | 5.942704545 | | | | |
| SLC16A6 | 7.43912 | 7.390255263 | 7.386244118 | 7.296981818 | | | | |
| LRRC40 | 6.164913333 | 6.141073684 | 6.113097059 | 6.023013636 | | | | |
| BC051070 | 5.77394 | 5.731926316 | 5.759560294 | 5.632954545 | | | | |
| LOC100045804 | 6.080636667 | 6.046034211 | 6.041739706 | 5.939822727 | | | | |
| HSH2D | 7.86715 | 7.806244737 | 7.842983824 | 7.726418182 | | | | |
| VPS26B | 6.30113 | 6.266228947 | 6.299214706 | 6.160686364 | | | | |
| ST6GAL2 | 5.81399 | 5.816073684 | 5.828707353 | 5.674245455 | | | | |
| TMPRSS11A | 5.94694 | 5.900107895 | 5.882957353 | 5.808109091 | | | | |
| RAB27A | 9.618303333 | 9.593155263 | 9.622351471 | 9.480636364 | | | | |
| LOC674087 | 8.063523333 | 8.043044737 | 8.033966176 | 7.926122727 | | | | |
| 1600012H06RIK | 7.32814 | 7.321276316 | 7.312647059 | 7.190854545 | | | | |
| 4733401O04RIK | 6.47715 | 6.413797368 | 6.449102941 | 6.340095455 | | | | |
| OLFR504 | 5.77475 | 5.783405263 | 5.745994118 | 5.638240909 | | | | |
| ADRA1B | 5.788323333 | 5.726486842 | 5.760369118 | 5.652722727 | | | | |
| RAB6B | 5.805626667 | 5.7864 | 5.766961765 | 5.670081818 | | | | |
| HBB-B1 | 13.32452667 | 13.28905789 | 13.28345588 | 13.18936818 | | | | |
| ARHGAP12 | 5.6783 | 5.657913158 | 5.653547059 | 5.543509091 | | | | |
| TYKI | 7.718213333 | 7.733778947 | 7.673405882 | 7.583704545 | | | | |
| LOC100048616 | 6.623486667 | 6.608734211 | 6.594576471 | 6.489290909 | | | | |
| 2600011C06RIK | 6.318883333 | 6.324634211 | 6.286382353 | 6.185263636 | | | | |
| BIRC6 | 6.316916667 | 6.256421053 | 6.264130882 | 6.184036364 | | | | |
| RGS19 | 6.648676667 | 6.608102632 | 6.584333824 | 6.516268132 | | | | |
| C130064E22RIK | 7.445486667 | 7.401763158 | 7.410882353 | 7.313245455 | | | | |
| ITK | 10.31404333 | 10.35670789 | 10.36085 | 10.18205455 | | | | |
| LCP2 | 6.589333333 | 6.593478947 | 6.556810294 | 6.45785 | | | | |
| RBM18 | 7.331313333 | 7.341076316 | 7.292019118 | 7.200204545 | | | | |
| ZAP70 | 7.124003333 | 7.130486842 | 7.106404412 | 6.993336364 | | | | |
| control_ILMN_1378612 | 5.722833333 | 5.724013158 | 5.700172059 | 5.592318182 | | | | |
| MAT2A | 8.35585 | 8.352134211 | 8.359186765 | 8.225568182 | | | | |
| 2210018M11RIK | 6.638536667 | 6.604639474 | 6.602507353 | 6.508409091 | | | | |
| ASH1L | 5.996946667 | 5.998094737 | 5.946575 | 5.8675 | | | | |
| LOXL3 | 5.826726667 | 5.771368421 | 5.800639706 | 5.697527273 | | | | |
| UTX | 9.849873333 | 9.822334211 | 9.816958824 | 9.722554545 | | | | |
| A730082L10RIK | 6.309363333 | 6.281157895 | 6.255379412 | 6.182745455 | | | | |
| ITGA6 | 6.613506667 | 6.588739474 | 6.588763235 | 6.48725 | | | | |
| control_ILMN_1378679 | 5.660046667 | 5.650981579 | 5.603908824 | 5.534195455 | | | | |
| 4930449I04RIK | 5.92482 | 5.89145 | 5.894451471 | 5.800354545 | | | | |
| TSC1 | 6.022403333 | 6.007705263 | 5.983791176 | 5.898672727 | | | | |
| MSH3 | 6.317296667 | 6.317907895 | 6.287680882 | 6.193777273 | | | | |
| 9330171B01RIK | 5.83447 | 5.777323684 | 5.814472059 | 5.710968182 | | | | |
| UGT2A1 | 5.621583333 | 5.633452632 | 5.610448529 | 5.498127273 | | | | |
| C130078D09RIK | 6.053696552 | 6.004831579 | 6.019788235 | 5.930827273 | | | | |
| RHBDF2 | 6.356373333 | 6.346123684 | 6.336120588 | 6.233854545 | | | | |
| INPP4B | 9.272156667 | 9.351981579 | 9.328983824 | 9.149936364 | | | | |
| NIPSNAP1 | 9.28918 | 9.308102632 | 9.269794118 | 9.167031818 | | | | |
| LPIN2 | 7.60717 | 7.597921053 | 7.581547059 | 7.485290909 | | | | |
| 4833420G17RIK | 6.1815 | 6.133973684 | 6.181452941 | 6.059827273 | | | | |
| NT5C | 10.14017667 | 10.13889474 | 10.09159412 | 10.0186 | | | | |
| D730033A03RIK | 5.94283 | 5.894176316 | 5.894086765 | 5.821409091 | | | | |
| 1700011H14RIK | 5.766446667 | 5.738492105 | 5.738245588 | 5.645190909 | | | | |
| 4921505C17RIK | 6.429206667 | 6.39755 | 6.381835294 | 6.309031818 | | | | |
| ZSCAN2 | 5.832116667 | 5.778686842 | 5.812569118 | 5.711959091 | | | | |
| ATL1 | 5.71863 | 5.665681579 | 5.666389706 | 5.5989 | | | | |
| COPZ1 | 6.3019 | 6.281852632 | 6.259785294 | 6.182704545 | | | | |
| 4921506M07RIK | 5.789413333 | 5.778978947 | 5.748717647 | 5.670604545 | | | | |
| 4921506J03RIK | 9.92524 | 9.871423684 | 9.893135294 | 9.807481818 | | | | |
| GADD45GIP1 | 5.926886667 | 5.9394 | 5.892926471 | 5.809209091 | | | | |
| RDH1 | 5.70993 | 5.703157895 | 5.681833824 | 5.592604545 | | | | |
| IKZF5 | 7.25512 | 7.225368421 | 7.230170588 | 7.138109091 | | | | |
| LOC385046 | 5.73528 | 5.711797368 | 5.694930882 | 5.618386364 | | | | |
| BC057371 | 5.73612 | 5.768484211 | 5.751522059 | 5.619290909 | | | | |
| 9830118G07RIK | 6.56715 | 6.525489474 | 6.530077941 | 6.450931818 | | | | |
| MRPS31 | 5.696653333 | 5.649865789 | 5.670588235 | 5.580590909 | | | | |
| 1600029O10RIK | 7.692803333 | 7.769444737 | 7.691802941 | 7.576986364 | | | | |
| OLFR125 | 5.710643333 | 5.670455263 | 5.696926471 | 5.594827273 | | | | |
| SPCS2 | 5.935356667 | 5.986028947 | 5.970344118 | 5.819986364 | | | | |
| BCL10 | 6.291966667 | 6.268534211 | 6.260922059 | 6.176790909 | | | | |
| BCL11B | 6.466986667 | 6.517447368 | 6.505741176 | 6.351922727 | | | | |
| control_ILMN_1379152 | 5.677653333 | 5.641942105 | 5.629272059 | 5.562736364 | | | | |
| DPPA4 | 5.814133333 | 5.774526316 | 5.790995588 | 5.699431818 | | | | |
| D12WSU95E | 9.425403333 | 9.463215789 | 9.425804412 | 9.310904545 | | | | |
| 1700045I19RIK | 5.913596667 | 5.870136842 | 5.885326471 | 5.799536364 | | | | |
| MKI67 | 5.71464 | 5.682371053 | 5.672839706 | 5.600581618 | | | | |
| HIRA | 5.71061 | 5.698692105 | 5.730233824 | 5.596768182 | | | | |
| BC016608 | 5.873616667 | 5.852886842 | 5.838388235 | 5.7599 | | | | |
| HIVEP3 | 5.8143 | 5.795202632 | 5.795891176 | 5.700604545 | | | | |
| CDKN1B | 6.319963333 | 6.323197368 | 6.290032353 | 6.206463636 | | | | |
| TMEM146 | 6.1685 | 6.148844737 | 6.132217647 | 6.056054545 | | | | |
| ELF2 | 6.475283333 | 6.457747368 | 6.474922059 | 6.362845455 | | | | |
| LOC100046343 | 8.632353333 | 8.628797368 | 8.607308824 | 8.521077273 | | | | |
| INSL6 | 9.453976667 | 9.436828947 | 9.438182353 | 9.34275 | | | | |
| TSSC1 | 7.696453333 | 7.670657895 | 7.728852941 | 7.585313636 | | | | |
| APOM | 6.10942 | 6.101231579 | 6.071398529 | 5.998286364 | | | | |
| BCORL1 | 6.14217 | 6.119342105 | 6.101613235 | 6.031768182 | | | | |
| KAT2B | 6.576886667 | 6.58595 | 6.563575 | 6.466563636 | | | | |
| DPPA1 | 5.75981 | 5.748876316 | 5.758391176 | 5.649590909 | | | | |
| LOC675899 | 11.88372667 | 11.89631579 | 11.89802353 | 11.77363636 | | | | |
| CDH23 | 5.970186667 | 5.945952632 | 5.945426471 | 5.860313636 | | | | |
| LOC384240 | 5.845473333 | 5.816294737 | 5.807029412 | 5.735695455 | | | | |
| PHIP | 8.148903333 | 8.24305 | 8.191986765 | 8.039681818 | | | | |
| ZCCHC11 | 5.953363333 | 5.943892105 | 5.934814706 | 5.844218182 | | | | |
| C030004B10RIK | 5.714183333 | 5.7017 | 5.707923529 | 5.605068182 | | | | |
| SH3BP2 | 10.99547 | 10.98236842 | 10.95497941 | 10.88651364 | | | | |
| 9030003C19RIK | 6.30629 | 6.278178947 | 6.271342647 | 6.197777273 | | | | |
| PTP4A2 | 8.967936667 | 9.012752632 | 8.951845588 | 8.859495455 | | | | |
| B230120H23RIK | 5.60376 | 5.592065789 | 5.612216176 | 5.495790909 | | | | |
| LOC630421 | 6.025783333 | 5.977681579 | 5.995302941 | 5.917995455 | | | | |
| MYO5C | 5.774123333 | 5.765497368 | 5.730282353 | 5.666459091 | | | | |
| A130027D22RIK | 6.198296667 | 6.165173684 | 6.183789706 | 6.090895455 | | | | |
| TMPO | 8.557416667 | 8.585160526 | 8.533298529 | 8.450218182 | | | | |
| HSD362 | 5.771093333 | 5.73905 | 5.744586765 | 5.663945455 | | | | |
| NDRG1 | 7.336333333 | 7.323686842 | 7.329026471 | 7.229204545 | | | | |
| RAB4A | 6.843623333 | 6.862610526 | 6.850907353 | 6.736786364 | | | | |
| RPL32 | 13.83211333 | 13.81921842 | 13.80899412 | 13.72541364 | | | | |
| A930025D01RIK | 6.41673 | 6.396328947 | 6.430195588 | 6.310840909 | | | | |
| NUFIP2 | 6.071443333 | 6.053294737 | 6.030458824 | 5.966290909 | | | | |
| LOC236831 | 12.44257567 | 12.44817105 | 12.42664118 | 12.33750455 | | | | |
| GPR12 | 5.7837 | 5.755857895 | 5.747101471 | 5.679290909 | | | | |
| GLRA1 | 5.690036667 | 5.6508 | 5.643054412 | 5.585631818 | | | | |
| TRPM1 | 5.66866 | 5.650436842 | 5.694383824 | 5.564486364 | | | | |
| MIS12 | 5.971716667 | 5.986276316 | 5.988970588 | 5.867713636 | | | | |
| USP47 | 6.113853333 | 6.0853 | 6.068192647 | 6.010090909 | | | | |
| DEFB14 | 5.79444 | 5.758081579 | 5.775191176 | 5.690909091 | | | | |
| 1600032L17RIK | 6.07718 | 6.053210526 | 6.091069118 | 5.97365 | | | | |
| HEATR5B | 6.279636667 | 6.262047368 | 6.250626471 | 6.176159091 | | | | |
| EP400 | 5.911806667 | 5.921736842 | 5.944972059 | 5.808472727 | | | | |
| C130075H21RIK | 5.863153333 | 5.838913158 | 5.868792647 | 5.76005 | | | | |
| MYB | 5.96478 | 5.935865789 | 5.950458824 | 5.861781818 | | | | |
| 2810402E24RIK | 6.330663333 | 6.285539474 | 6.296026471 | 6.227672727 | | | | |
| DLGAP2 | 5.728533333 | 5.739528947 | 5.718060294 | 5.625627273 | | | | |
| COL23A1 | 7.128406667 | 7.137160526 | 7.128491176 | 7.025504545 | | | | |
| FAAH | 5.98956 | 5.964115789 | 5.946645588 | 5.886959091 | | | | |
| DLC1 | 5.824196667 | 5.799747368 | 5.803795588 | 5.721713636 | | | | |
| E230016F22RIK | 5.729066667 | 5.684757895 | 5.684539706 | 5.627031818 | | | | |
| 1700073E17RIK | 5.641803333 | 5.631934211 | 5.628777941 | 5.539877273 | | | | |
| MDM2 | 10.50170333 | 10.45636579 | 10.46051324 | 10.39990909 | | | | |
| E530016P10RIK | 6.104856667 | 6.070465789 | 6.095805882 | 6.003086364 | | | | |
| ZFP212 | 6.38679 | 6.384781579 | 6.401176471 | 6.285086364 | | | | |
| IFI204 | 5.521196667 | 5.489834211 | 5.486161765 | 5.420104545 | | | | |
| TRPV3 | 5.62817 | 5.598486842 | 5.597452941 | 5.527354545 | | | | |
| SEC1 | 5.754276667 | 5.740523684 | 5.749694118 | 5.653522727 | | | | |
| 9330133O14RIK | 6.23197 | 6.226260526 | 6.205191176 | 6.131222727 | | | | |
| GALNT1 | 7.85888 | 7.857452632 | 7.895080882 | 7.758145455 | | | | |
| FOXJ3 | 8.670526667 | 8.657994737 | 8.647885294 | 8.569918182 | | | | |
| 1810005K13RIK | 5.77925 | 5.75325 | 5.740070588 | 5.678704545 | | | | |
| CLASP1 | 6.05413 | 6.064752632 | 6.034072059 | 5.95395 | | | | |
| 6030408K21RIK | 5.669393333 | 5.666268421 | 5.640398529 | 5.569368182 | | | | |
| A930008A22RIK | 8.380963333 | 8.430597368 | 8.393158824 | 8.281059091 | | | | |
| 1810007M14RIK | 6.890543333 | 6.891115789 | 6.859664706 | 6.790654545 | | | | |
| BC003331 | 7.384963333 | 7.351997368 | 7.367855882 | 7.285531818 | | | | |
| LOC382061 | 10.37113 | 10.37368684 | 10.40468824 | 10.27182273 | | | | |
| NA112 | 8.314633333 | 8.302757895 | 8.275223529 | 8.215740909 | | | | |
| OLFR782 | 5.737763333 | 5.716676316 | 5.74 | 5.639059091 | | | | |
| CPEB2 | 5.658066667 | 5.666544737 | 5.648361765 | 5.559427273 | | | | |
| 2510049I19RIK | 6.335453333 | 6.332197368 | 6.311452941 | 6.236859091 | | | | |
| LCN3 | 5.884476667 | 5.856815789 | 5.859325 | 5.786277273 | | | | |
| 9930021J03RIK | 5.63169 | 5.637307895 | 5.609635294 | 5.53385 | | | | |
| LOC268939 | 6.025216667 | 6.026355263 | 5.989661765 | 5.928159091 | | | | |
| IGHV14S3_{—} | 6.005066667 | 6.045584211 | 6.044322059 | 5.908227273 | | | | |
| X03573$M12991X03573_{—} | | | | | | | | |
| IG_HEAVY_{—} | | | | | | | | |
| VARIABLE_14S3_203 | | | | | | | | |
| 4930524L23RIK | 5.51084 | 5.509984211 | 5.492057353 | 5.414113636 | | | | |
| NANP | 5.710083333 | 5.689892105 | 5.685992647 | 5.613513636 | | | | |
| PCSK2 | 5.669273333 | 5.668607895 | 5.653467647 | 5.572977273 | | | | |
| C87414 | 5.990493333 | 5.997397368 | 5.972451471 | 5.8942 | | | | |
| TMEFF2 | 5.677973333 | 5.660439474 | 5.649566176 | 5.581804545 | | | | |
| MIA3 | 5.843996667 | 5.849473684 | 5.862173529 | 5.747836364 | | | | |
| EG546913 | 5.761706667 | 5.767334211 | 5.766423529 | 5.665590909 | | | | |
| A630040J04RIK | 5.993063333 | 6.012613158 | 5.997267647 | 5.897040909 | | | | |
| HARS | 6.238436667 | 6.216931579 | 6.211557353 | 6.142640909 | | | | |
| HRK | 5.75883 | 5.723236842 | 5.720327941 | 5.6631 | | | | |
| LOC100045437 | 5.742806667 | 5.732802632 | 5.739961765 | 5.647127273 | | | | |
| AFMID | 6.246823333 | 6.234655263 | 6.225633824 | 6.151559091 | | | | |
| PMS1 | 5.817356667 | 5.801978947 | 5.805682353 | 5.722281818 | | | | |
| ID2 | 6.11898 | 6.112697368 | 6.094567647 | 6.024290909 | | | | |
| FABP2 | 5.574673333 | 5.581957895 | 5.582427941 | 5.48005 | | | | |
| ATG12 | 8.973293333 | 8.94255 | 8.962186765 | 8.87895 | | | | |
| FBXW13 | 5.958916667 | 5.941 | 5.944770588 | 5.864777273 | | | | |
| TDH | 5.632846667 | 5.605236842 | 5.600979412 | 5.538713636 | | | | |
| CALCRL | 5.57102 | 5.552502632 | 5.569420588 | 5.476895455 | | | | |
| IL1F9 | 5.66582 | 5.667273684 | 5.643604412 | 5.571768182 | | | | |
| SYT10 | 5.726353333 | 5.6979 | 5.716591176 | 5.632418182 | | | | |
| MEX3A | 6.931541379 | 6.896444737 | 6.913239706 | 6.837772727 | | | | |
| PRICKLE2 | 5.850416667 | 5.857647368 | 5.858801471 | 5.756763636 | | | | |
| 5830415L20RIK | 7.799203333 | 7.800326316 | 7.79705 | 7.705868182 | | | | |
| GAPVD1 | 9.875493333 | 9.862042105 | 9.838317647 | 9.782222727 | | | | |
| LOC381770 | 6.123156667 | 6.095863158 | 6.112223529 | 6.03 | | | | |
| TATDN3 | 5.724313333 | 5.717452632 | 5.693994118 | 5.631331818 | | | | |
| KRT32 | 5.993876667 | 5.999742105 | 6.010070588 | 5.901004545 | | | | |
| LRRC18 | 5.526923333 | 5.530942105 | 5.542358824 | 5.434140909 | | | | |
| NIN | 8.5343 | 8.546615789 | 8.533970588 | 8.442313636 | | | | |
| 5830411J07RIK | 5.85217 | 5.849081579 | 5.837338235 | 5.760313636 | | | | |
| EEFSEC | 5.65683 | 5.664715789 | 5.654432353 | 5.565031818 | | | | |
| SPDYB | 5.668073333 | 5.642892105 | 5.640694118 | 5.576868182 | | | | |
| RNF150 | 5.896763333 | 5.955207895 | 5.930469118 | 5.805768182 | | | | |
| SVNGR4 | 5.656433333 | 5.638892105 | 5.646776471 | 5.565595455 | | | | |
| 6030408816RIK | 6.25216 | 6.227231579 | 6.230682353 | 6.161786364 | | | | |
| C130039E17RIK | 5.731136667 | 5.703355263 | 5.729467647 | 5.640990909 | | | | |
| KRIT1 | 5.64008 | 5.672247368 | 5.683764706 | 5.549959091 | | | | |
| control_ILMN_1378774 | 5.669826667 | 5.666792105 | 5.707342647 | 5.580013636 | | | | |
| PAPPA | 5.948626667 | 5.924163158 | 5.918261765 | 5.858904545 | | | | |
| 1700016L04RIK | 5.716706667 | 5.719863158 | 5.692752941 | 5.627036364 | | | | |
| C130072C03RIK | 6.081323333 | 6.089007895 | 6.075160294 | 5.992027273 | | | | |
| OLFR564 | 5.610586667 | 5.597744737 | 5.591648529 | 5.521359091 | | | | |
| 1700016G05RIK | 5.763153333 | 5.776560526 | 5.780139706 | 5.674022727 | | | | |
| RgenericRIP1 | 5.954443333 | 5.936397368 | 5.934783824 | 5.865727273 | | | | |
| K20D4 | 5.705186667 | 5.706357895 | 5.688022059 | 5.616690909 | | | | |
| 4922502D21RIK | 5.802656667 | 5.779581579 | 5.773558824 | 5.714322727 | | | | |
| GM949 | 5.67855 | 5.668557895 | 5.662460294 | 5.590413636 | | | | |
| DLG4 | 6.05743 | 6.053318421 | 6.044314706 | 5.969368182 | | | | |
| PTPN22 | 5.784923333 | 5.781565789 | 5.804307353 | 5.697240909 | | | | |
| MEDI | 5.69954 | 5.688523684 | 5.672022059 | 5.612195455 | | | | |
| HPN | 5.78737 | 5.775389474 | 5.793452941 | 5.700045455 | | | | |
| USP47 | 6.331733333 | 6.327818421 | 6.377101471 | 6.24445 | | | | |
| CTDSPL2 | 6.688386667 | 6.693771053 | 6.710814706 | 6.601177273 | | | | |
| PARP11 | 5.620256667 | 5.624031579 | 5.609260294 | 5.533118182 | | | | |
| LOC381561 | 13.19958667 | 13.16311842 | 13.17164853 | 13.1126 | | | | |
| V1RD9 | 5.658806667 | 5.657992105 | 5.697108824 | 5.572222727 | | | | |
| SPN | 5.89838 | 5.884368421 | 5.888555882 | 5.811963636 | | | | |
| KCNK4 | 5.664273333 | 5.677852632 | 5.658711765 | 5.577881818 | | | | |
| PAX6 | 5.669473333 | 5.681165789 | 5.640964706 | 5.583190909 | | | | |
| COPS3 | 6.04268 | 6.063476316 | 6.029414706 | 5.956536364 | | | | |
| OLFR894 | 5.7664 | 5.789839474 | 5.766469118 | 5.6803 | | | | |
| 9530053H22 | 5.551996667 | 5.571878947 | 5.531242647 | 5.465995455 | | | | |
| 5830472H07RIK | 6.253593333 | 6.282686842 | 6.240239706 | 6.167636364 | | | | |
| D930007M19RIK | 6.347126667 | 6.356386842 | 6.333030882 | 6.261340909 | | | | |
| D130078K04RIK | 5.92102 | 5.905407895 | 5.957402941 | 5.835468182 | | | | |
| LOC384287 | 5.600046667 | 5.584278947 | 5.612377941 | 5.514790909 | | | | |
| LOC100048049 | 5.83809 | 5.839768421 | 5.810255882 | 5.752986364 | | | | |
| 1700112H15RIK | 5.602223333 | 5.589636842 | 5.628055882 | 5.517240909 | | | | |
| RNF17 | 5.560576667 | 5.571157895 | 5.537982353 | 5.475609091 | | | | |
| NARF | 7.168896667 | 7.19995 | 7.223792647 | 7.084013636 | | | | |
| HSPA1B | 5.990493333 | 6.0012 | 5.989733824 | 5.905659091 | | | | |
| B930071N01RIK | 5.752686667 | 5.753815789 | 5.764302941 | 5.668054545 | | | | |
| SEMA3E | 5.622603333 | 5.610747368 | 5.600951471 | 5.538277273 | | | | |
| GALNT6 | 5.865516667 | 5.857652632 | 5.872851471 | 5.781409091 | | | | |
| NCOA2 | 5.96424 | 5.957523684 | 5.963505882 | 5.880186364 | | | | |
| LOC385027 | 5.646326667 | 5.633852632 | 5.665861765 | 5.562377273 | | | | |
| SPRR2K | 5.623606667 | 5.644615789 | 5.662151471 | 5.539818182 | | | | |
| STAT4 | 5.838816667 | 5.842352632 | 5.836001471 | 5.755045455 | | | | |
| 1700006H02RIK | 5.87839 | 5.868286842 | 5.861352941 | 5.794681818 | | | | |
| OLFR463 | 5.806456667 | 5.809223684 | 5.804622059 | 5.722768182 | | | | |
| LOC637082 | 5.796636667 | 5.795723684 | 5.804989706 | 5.712972727 | | | | |
| CHMP7 | 9.48094 | 9.490673684 | 9.491242647 | 9.397440909 | | | | |
| AGPAT5 | 5.9402 | 5.938721053 | 5.925610294 | 5.856831818 | | | | |
| GPRIN3 | 5.649423333 | 5.661078947 | 5.627986765 | 5.566109091 | | | | |
| R3HDM1 | 6.22318 | 6.204939474 | 6.228605882 | 6.140118182 | | | | |
| GM239 | 5.75041 | 5.755331579 | 5.730616176 | 5.667736364 | | | | |
| genericC | 5.638906667 | 5.663857895 | 5.675654412 | 5.556236364 | | | | |
| GUCY2C | 5.75101 | 5.748381579 | 5.739895588 | 5.668413636 | | | | |
| DNAJC28 | 5.995306657 | 5.987768421 | 5.998058824 | 5.912995455 | | | | |
| A630057J21RIK | 5.72068 | 5.759102632 | 5.710363235 | 5.638481818 | | | | |
| PDK2 | 5.95138 | 5.955163158 | 5.936645588 | 5.869513636 | | | | |
| 1110032A03RIK | 5.859373333 | 5.846365789 | 5.852595588 | 5.777881818 | | | | |
| 2310016E02RIK | 5.7544 | 5.743484211 | 5.742011765 | 5.672968182 | | | | |
| C330011J12RIK | 5.719083333 | 5.714607895 | 5.721825 | 5.637795455 | | | | |
| JRKL | 5.823186667 | 5.821102632 | 5.816498529 | 5.741981818 | | | | |
| ZFP120 | 5.540176667 | 5.535910526 | 5.536211765 | 5.459018182 | | | | |
| KISS1 | 5.697123333 | 5.689555263 | 5.726642647 | 5.6162 | | | | |
| RTN3 | 5.90647 | 5.888605263 | 5.896657353 | 5.825581818 | | | | |
| AW822252 | 5.76074 | 5.763905263 | 5.74905 | 5.679863636 | | | | |
| LRRC39 | 5.708436667 | 5.715042105 | 5.711988235 | 5.627659091 | | | | |
| SCNN1B | 5.610303333 | 5.606213158 | 5.616464706 | 5.529563636 | | | | |
| DLX6 | 5.75542 | 5.755936842 | 5.765739706 | 5.674818182 | | | | |
| GM438 | 5.745906667 | 5.720597368 | 5.730714706 | 5.665440909 | | | | |
| LOC236397 | 5.832173333 | 5.820278947 | 5.838497059 | 5.751754545 | | | | |
| LOC381448 | 12.88779333 | 12.89468947 | 12.90875882 | 12.80749091 | | | | |
| CCDC104 | 5.84063 | 5.859457895 | 5.831667647 | 5.760527273 | | | | |
| LOC279638 | 6.0283 | 6.041955263 | 6.022392647 | 5.948613636 | | | | |
| MADD | 5.59808 | 5.593768421 | 5.583701471 | 5.518418182 | | | | |
| LOC333749 | 5.940406667 | 5.940531579 | 5.938926471 | 5.860786364 | | | | |
| AGT | 5.643776667 | 5.644352632 | 5.633804412 | 5.564418182 | | | | |
| C77370 | 5.701306667 | 5.713702632 | 5.694325 | 5.621972727 | | | | |
| LOC382454 | 5.770946667 | 5.769571053 | 5.761170588 | 5.692477273 | | | | |
| 9130601C02RIK | 5.722513333 | 5.701268421 | 5.723889706 | 5.644172727 | | | | |
| B230216G23RIK | 5.642603333 | 5.6564 | 5.625255882 | 5.56435 | | | | |
| BMPR1B | 5.720253333 | 5.742613158 | 5.732064706 | 5.642009091 | | | | |
| LOC100046568 | 5.87139 | 5.907021053 | 5.888016176 | 5.793381818 | | | | |
| A130003P22RIK | 6.037656667 | 6.046863158 | 6.042676471 | 5.959827273 | | | | |
| CFL2 | 6.263776667 | 6.252907895 | 6.246395588 | 6.18595 | | | | |
| control_ILMN_1378775 | 5.630993333 | 5.667786842 | 5.661682353 | 5.553213636 | | | | |
| TCFAP2C | 5.818516667 | 5.827960526 | 5.820864706 | 5.740859091 | | | | |
| BHLHB3 | 5.618706667 | 5.606023684 | 5.621429412 | 5.541054545 | | | | |
| UGDH | 5.862666667 | 5.856173684 | 5.860154118 | 5.78535 | | | | |
| control_ILMN_1379205 | 5.71972 | 5.711386842 | 5.720311765 | 5.642781818 | | | | |
| CRLF1 | 5.672033333 | 5.679189474 | 5.647736765 | 5.595127273 | | | | |
| SOX2 | 5.68709 | 5.678613158 | 5.674401471 | 5.610327273 | | | | |
| LOC229152 | 5.61709 | 5.608715789 | 5.604654412 | 5.540372727 | | | | |
| 2210008F06RIK | 5.65113 | 5.654563158 | 5.650383824 | 5.574586364 | | | | |
| 9530013H16R1K | 5.806686667 | 5.790413158 | 5.782267647 | 5.730295455 | | | | |
| MACROD2 | 5.871886667 | 5.861818421 | 5.854417647 | 5.795531818 | | | | |
| ZBTB44 | 5.652506667 | 5.634860526 | 5.641651471 | 5.576604545 | | | | |
| LOC209474 | 5.677973333 | 5.671436842 | 5.679282353 | 5.602077273 | | | | |
| OLFR1158 | 5.90849 | 5.900489474 | 5.911680882 | 5.832872727 | | | | |
| LOC383612 | 5.58247 | 5.581242105 | 5.568833824 | 5.506859091 | | | | |
| NFAT5 | 5.82748 | 5.836571053 | 5.815392647 | 5.751886364 | | | | |
| IGHVS515_{—} | 5.96497 | 5.996957895 | 5.952066176 | 5.889468182 | | | | |
| AF290966_IG_HEAVY_{—} | | | | | | | | |
| VARIABLE_5515_49 | | | | | | | | |
| ELMO2 | 5.873053333 | 5.918702632 | 5.906870588 | 5.79765 | | | | |
| 3110079O15RIK | 5.66052 | 5.669471053 | 5.641986765 | 5.585468182 | | | | |
| GFRA3 | 5.61845 | 5.603889474 | 5.615789706 | 5.543463636 | | | | |
| OSBPL6 | 5.831956667 | 5.852805263 | 5.8511 | 5.757227273 | | | | |
| DLG1 | 5.726496667 | 5.737444737 | 5.733544118 | 5.652140909 | | | | |
| D130063P19RIK | 5.772983333 | 5.771063158 | 5.767867647 | 5.698895455 | | | | |
| EG634762 | 5.778483333 | 5.806255263 | 5.772413235 | 5.704395455 | | | | |
| 4833419KD8RIK | 5.580176667 | 5.582989474 | 5.587622059 | 5.506263636 | | | | |
| D130061E05RIK | 5.734043333 | 5.723581579 | 5.722885294 | 5.660136364 | | | | |
| TMEM219 | 6.148696667 | 6.176007895 | 6.157932353 | 6.074918182 | | | | |
| OA1 | 5.56251 | 5.581981579 | 5.551513235 | 5.488740909 | | | | |
| OLFR1180 | 5.670083333 | 5.663052632 | 5.658069118 | 5.5964 | | | | |
| EFCAB1 | 5.710406667 | 5.73445 | 5.719770588 | 5.636781318 | | | | |
| control_ILMN_1378664 | 5.725506667 | 5.737144737 | 5.743244118 | 5.652136364 | | | | |
| BRPF1 | 8.94028 | 8.991239474 | 8.957891176 | 8.866990909 | | | | |
| CORO1B | 11.35823333 | 11.37271842 | 11.35536912 | 11.28498636 | | | | |
| LOC235971 | 5.526783333 | 5.511610526 | 5.505255882 | 5.453613636 | | | | |
| LOC383940 | 5.806973333 | 5.816121053 | 5.804944118 | 5.733954545 | | | | |
| RNF207 | 5.854813333 | 5.893897368 | 5.863382353 | 5.781881818 | | | | |
| 1700081L16RIK | 5.581866667 | 5.561147368 | 5.560519118 | 5.509463636 | | | | |
| OLFR750 | 5.666133333 | 5.666073684 | 5.646426471 | 5.593809091 | | | | |
| LOC385626 | 6.048793333 | 6.053842105 | 6.045866176 | 5.977095455 | | | | |
| LOC384513 | 5.711216667 | 5.710447368 | 5.704545588 | 5.639831818 | | | | |
| LOC385059 | 5.548423333 | 5.543284211 | 5.538563235 | 5.477331818 | | | | |
| 4930571C24RIK | 5.43153 | 5.426878947 | 5.413977941 | 5.360531818 | | | | |
| PPP1R14D | 5.628493333 | 5.633902632 | 5.610638235 | 5.557659091 | | | | |
| GJA7 | 5.87617 | 5.869497368 | 5.867842647 | 5.8054 | | | | |
| OLFR1085 | 5.636943333 | 5.654255263 | 5.642222059 | 5.566422727 | | | | |
| NEUROD4 | 5.671153333 | 5.669668421 | 5.670869118 | 5.60065 | | | | |
| LTBP1 | 5.68037 | 5.683613158 | 5.694298529 | 5.610181818 | | | | |
| control_ILMN_1378571 | 5.820813333 | 5.820989474 | 5.812435294 | 5.750686364 | | | | |
| PIK3R4 | 5.89115 | 5.900139474 | 5.881456824 | 5.821327273 | | | | |
| 2210003I03RIK | 5.69808 | 5.689707895 | 5.693454412 | 5.628409091 | | | | |
| 29D0003A17RIK | 6.01923 | 6.020481579 | 6.032035294 | 5.949854545 | | | | |
| EZH2 | 5.613896667 | 5.618357895 | 5.625351471 | 5.544813636 | | | | |
| BTF3L4 | 5.68815 | 5.685586842 | 5.681852941 | 5.619090909 | | | | |
| LOC676672 | 5.804406667 | 5.811518421 | 5.824616176 | 5.735581818 | | | | |
| control_ILMN_1379333 | 5.61851 | 5.653657895 | 5.620225 | 5.549695455 | | | | |
| B230323D24RIK | 5.808683333 | 5.799486842 | 5.809979412 | 5.739909091 | | | | |
| 3110052M02RIK | 5.663506667 | 5.668044737 | 5.650769118 | 5.505422727 | | | | |
| ENSMUSG00000071316 | 5.765793333 | 5.770668421 | 5.766132353 | 5.697813636 | | | | |
| LOC384215 | 5.820696667 | 5.862371053 | 5.829138235 | 5.752890909 | | | | |
| D530031C13RIK | 5.739493333 | 5.733907895 | 5.728495588 | 5.672031818 | | | | |
| LOC277422 | 5.73676 | 5.733102632 | 5.729133824 | 5.669509091 | | | | |
| A530088H08RIK | 5.813696667 | 5.825318421 | 5.837829412 | 5.746622727 | | | | |
| HEATR5A | 5.68794 | 5.696721053 | 5.694547059 | 5.621004545 | | | | |
| 6430527G18RIK | 5.60759 | 5.649342105 | 5.618372059 | 5.540731818 | | | | |
| TRPC6 | 5.624033333 | 5.621573684 | 5.632232353 | 5.557395455 | | | | |
| GM362 | 5.620813333 | 5.632378947 | 5.638714706 | 5.554177273 | | | | |
| PCDHGA8 | 5.78089 | 5.776236842 | 5.781673529 | 5.714854545 | | | | |
| LOC620009 | 5.591003333 | 5.607339474 | 5.618951471 | 5.525318182 | | | | |
| LOC100043389 | 5.668326667 | 5.691036842 | 5.675438235 | 5.602663636 | | | | |
| SMARCA2 | 5.619723333 | 5.630702632 | 5.616189706 | 5.554095455 | | | | |
| A230066D03RIK | 5.923233333 | 5.915707895 | 5.912572059 | 5.857631818 | | | | |
| UTX | 5.6994 | 5.696263158 | 5.699866176 | 5.633977273 | | | | |
| OLFR1230 | 5.64594 | 5.643160526 | 5.655157353 | 5.580931818 | | | | |
| CREBZF | 5.673313333 | 5.683676316 | 5.660316176 | 5.608613636 | | | | |
| RARRES1 | 5.584843333 | 5.583405263 | 5.601169118 | 5.52025 | | | | |
| D130050A01RIK | 5.641893333 | 5.638021053 | 5.632995588 | 5.577468182 | | | | |
| LOC382783 | 5.531793333 | 5.538615789 | 5.536345588 | 5.467895455 | | | | |
| C430020E23RIK | 5.892203333 | 5.918694737 | 5.905788235 | 5.829490909 | | | | |
| ADAMTSL3 | 5.798876667 | 5.823873684 | 5.806304412 | 5.736231818 | | | | |
| SCL0070954.1_214 | 5.810163333 | 5.804763158 | 5.821651471 | 5.747618182 | | | | |
| PTCHD2 | 5.826433333 | 5.826802632 | 5.830785294 | 5.763954545 | | | | |
| CNTN2 | 5.713903333 | 5.724568421 | 5.729119118 | 5.651654545 | | | | |
| 6720487G11RIK | 5.956016667 | 5.954921053 | 5.948966176 | 5.895059091 | | | | |
| SERPINB3D | 5.745123333 | 5.752734211 | 5.740894118 | 5.685531818 | | | | |
| CRAT | 5.918913333 | 5.942655263 | 5.92075 | 5.859636364 | | | | |
| 4921522P10RIK | 5.777803333 | 5.795607895 | 5.792822059 | 5.719963636 | | | | |
| A830021M18 | 5.659886667 | 5.664492105 | 5.661938235 | 5.602090909 | | | | |
| RTN2 | 5.9209 | 5.963881579 | 5.939469118 | 5.864890909 | | | | |
| RPL10A | 14.66292333 | 14.65870789 | 14.65606176 | 14.60712273 | | | | |
| LOC382760 | 5.657563333 | 5.659857895 | 5.650822059 | 5.601886364 | | | | |
| 4930543E05RIK | 5.678906667 | 5.690528947 | 5.681104412 | 5.6236 | | | | |
| 5730599A22RIK | 5.674163333 | 5.691197368 | 5.674958824 | 5.620590909 | | | | |
| UP IN GA AND REF ONLY | | | | | | | | |
| C130079K02RIK | 7.22574 | 7.544463158 | 7.531626471 | 7.306454545 | | | | |
| ALS2CL | 7.116023333 | 7.394581579 | 7.392775 | 7.205686364 | | | | |
| 2810410P22RIK | 7.714383333 | 8.011573684 | 7.979019118 | 7.8124 | Pattern Represented by Data: | | | |
| HNRNPK | 9.7988 | 10.07715 | 10.05100735 | 9.913459091 | | | | |
| TRAT1 | 7.813176667 | 8.078084211 | 8.055941176 | 7.793545455 | | x | x | |
| LSG1 | 7.035183333 | 7.257968421 | 7.277522059 | 7.047777273 | x | | | x |
| RAB6 | 7.261916667 | 7.466060526 | 7.498622059 | 7.357177273 | Medium | Reference | GA | generic |
| ARL5A | 7.797843333 | 8.091231579 | 8.028401471 | 7.893445455 | | | | |
| OTTMUSG00000005523 | 6.737806667 | 6.952839474 | 6.959511765 | 6.774009091 | | | | |
| RN185 | 9.738126667 | 10.04179474 | 9.958561765 | 9.800913636 | | | | |
| A830083H19RIK | 7.416416667 | 7.6885 | 7.635472059 | 7.464036364 | | | | |
| FYB | 8.031866667 | 8.256457895 | 8.250267647 | 8.0774 | | | | |
| PUM2 | 8.49848 | 8.733373684 | 8.716505882 | 8.539704545 | | | | |
| SCL0002368.1_75 | 8.83479 | 9.028768421 | 9.052716176 | 8.814618182 | | | | |
| COX7A1 | 6.94444 | 7.100284211 | 7.161101471 | 6.989281818 | | | | |
| NPC2 | 9.368056667 | 9.561731579 | 9.575010294 | 9.4518 | | | | |
| SAMHD1 | 11.84208667 | 12.07217895 | 12.04837794 | 11.93060909 | | | | |
| CLCN3 | 8.224926667 | 8.427963158 | 8.430619118 | 8.2973 | | | | |
| PTPN1 | 9.086276667 | 9.362913158 | 9.291763235 | 9.143836364 | | | | |
| CBFB | 6.44466 | 6.629710526 | 6.648410294 | 6.525068182 | | | | |
| 5830457O10RIK | 6.626373333 | 6.803589474 | 6.824932353 | 6.691472727 | | | | |
| HIST1H2AG | 9.87061 | 10.02252105 | 10.067625 | 9.906631818 | | | | |
| LOC383706 | 8.164186667 | 8.320342105 | 8.360444118 | 8.219022727 | | | | |
| TPM4 | 7.28479 | 7.536536842 | 7.481042647 | 7.387468182 | | | | |
| CYB5 | 11.01746667 | 11.17962895 | 11.21147353 | 11.06824091 | | | | |
| TOP2A | 6.89295 | 7.115813158 | 7.084679412 | 6.964609091 | | | | |
| DCUN1D5 | 9.349493333 | 9.563021053 | 9.534638235 | 9.392522727 | | | | |
| PPP1CB | 7.532633333 | 7.763778947 | 7.717295588 | 7.616245455 | | | | |
| PJA1 | 6.55579 | 6.763297368 | 6.736463235 | 6.640472727 | | | | |
| CD247 | 7.05133 | 7.215657895 | 7.226701471 | 7.088009091 | | | | |
| RNF8 | 6.676843333 | 6.857478947 | 6.852086765 | 6.755077273 | | | | |
| TRMT2A | 6.52321 | 6.684289474 | 6.694272059 | 6.565359091 | | | | |
| ARL1 | 9.743456667 | 9.9474 | 9.913035294 | 9.828659091 | | | | |
| 4930539E08RIK | 6.088663333 | 6.262042105 | 6.255341176 | 6.113204545 | | | | |
| CXCR6 | 6.669376667 | 6.827515789 | 6.833716176 | 6.712486364 | | | | |
| TMPO | 8.46599 | 8.623305263 | 8.629933824 | 8.530504545 | | | | |
| IRF1 | 10.31889 | 10.48616842 | 10.48073382 | 10.32447727 | | | | |
| ACTN4 | 6.448333333 | 6.604534211 | 6.608894118 | 6.453640909 | | | | |
| WDR45L | 7.205136667 | 7.360331579 | 7.364545588 | 7.240713636 | | | | |
| ZFP281 | 8.814456667 | 9.007431579 | 8.973852941 | 8.863868182 | | | | |
| D6WSU163E | 6.423555667 | 6.621960526 | 6.581777941 | 6.499395455 | | | | |
| NUDCD2 | 6.944946667 | 7.134152632 | 7.101964706 | 6.987381818 | | | | |
| H2-T23 | 10.62381 | 10.76490263 | 10.77933382 | 10.67458636 | | | | |
| LOC667005 | 6.864946667 | 7.033302632 | 7.019588235 | 6.847404545 | | | | |
| EIF3B | 6.22973 | 6.388731579 | 6.382969118 | 6.276936364 | | | | |
| ELAC2 | 6.199506667 | 6.348668421 | 6.350842647 | 6.22455 | | | | |
| GTL7 | 7.284363333 | 7.47345 | 7.434189706 | 7.326722727 | | | | |
| LOC100046320 | 7.5542 | 7.737257895 | 7.703685294 | 7.607563636 | | | | |
| PARP14 | 10.98116 | 11.14566053 | 11.12864853 | 11.01771364 | | | | |
| PK3 | 6.16902 | 6.306352632 | 6.316370588 | 6.225122727 | | | | |
| ZZZ3 | 6.03378 | 6.146336842 | 6.180801471 | 6.068531818 | | | | |
| LOC100041450 | 6.805983333 | 6.921857895 | 6.952891176 | 6.835990909 | | | | |
| WAPAL | 6.27658 | 6.434936842 | 6.423070588 | 6.340836364 | | | | |
| 8030402P03RIK | 7.633246667 | 7.750686842 | 7.775892647 | 7.655845455 | | | | |
| A630098G22RIK | 6.98317 | 7.150939474 | 7.12385 | 7.044545455 | | | | |
| CSF1 | 6.24299 | 6.430692105 | 5.383498529 | 6.296836364 | | | | |
| FBXO33 | 8.757316667 | 8.881023684 | 8.897601471 | 8.793440909 | | | | |
| SLCO3A1 | 6.011396667 | 6.150071053 | 6.150582353 | 6.029604545 | | | | |
| PDLIM1 | 9.389206667 | 9.494944737 | 9.528047059 | 9.380759091 | | | | |
| 2810008M24RIK | 9.577263333 | 9.719655263 | 9.714169118 | 9.612672727 | | | | |
| 1700025G04RIK | 7.13209 | 7.290965789 | 7.268439706 | 7.1935 | | | | |
| BCL11B | 7.475326667 | 7.627081579 | 7.608722059 | 7.374281818 | | | | |
| D630022O22RIK | 6.197413333 | 6.334594737 | 6.330180882 | 6.235186364 | | | | |
| DMBT1 | 5.65465 | 5.791228947 | 5.787077941 | 5.706268182 | | | | |
| MAD2L1BP | 9.83867 | 9.946563158 | 9.970585294 | 9.864663636 | | | | |
| IRF1 | 10.39029333 | 10.53875526 | 10.52146618 | 10.39822727 | | | | |
| ETS1 | 7.680986667 | 7.843268421 | 7.811420588 | 7.700831818 | | | | |
| 1810013L24RIK | 7.528863333 | 7.691768421 | 7.658367647 | 7.581836364 | | | | |
| TOR1A | 7.383143333 | 7.495473684 | 7.511541176 | 7.425604545 | | | | |
| TRAT1 | 6.647076667 | 6.785913158 | 6.773917647 | 6.622813636 | | | | |
| PABPC1 | 6.85638 | 7.040826316 | 6.981691176 | 6.861254545 | | | | |
| H2AFZ | 11.80579667 | 11.93475 | 11.93059412 | 11.86225909 | | | | |
| ZDHHC21 | 8.437696667 | 8.5418 | 8.562170588 | 8.449168182 | | | | |
| ZNF24 | 6.048016667 | 6.124986842 | 6.171314706 | 6.028518182 | | | | |
| ZWILCH | 6.168553333 | 6.305978947 | 6.289795588 | 6.210068182 | | | | |
| AP1B1 | 10.82644667 | 10.95238421 | 10.94691618 | 10.87835 | | | | |
| 2500002A22RIK | 6.55427 | 6.705039474 | 6.673707353 | 6.569454545 | | | | |
| LOC380707 | 9.90633 | 10.03282105 | 10.02428529 | 9.90435 | | | | |
| TNP2 | 5.844083333 | 5.988826316 | 5.958627941 | 5.884845455 | | | | |
| GTF3C5 | 5.814863333 | 5.905071053 | 5.927685294 | 5.83125 | | | | |
| HIST1H2AK | 12.67005667 | 12.80842105 | 12.78147206 | 12.66135909 | | | | |
| RNGTT | 6.190413333 | 6.291310526 | 6.301817647 | 6.194254545 | | | | |
| VPS37B | 10.44526333 | 10.55398421 | 10.55639412 | 10.39754091 | | | | |
| F830048D03RIK | 6.689073333 | 6.771521053 | 6.799188235 | 6.670236364 | | | | |
| USF1 | 6.804973333 | 6.921971053 | 6.914854412 | 6.826927273 | | | | |
| HEATR3 | 5.744316667 | 5.838378947 | 5.853311765 | 5.734418182 | | | | |
| STIL | 5.61879 | 5.685102632 | 5.726732353 | 5.610486364 | | | | |
| FEM1C | 7.96895 | 8.091639474 | 8.076245588 | 8.010295455 | | | | |
| EG666756 | 10.67756 | 10.81662632 | 10.78483529 | 10.70416364 | | | | |
| PIBF1 | 6.849893333 | 6.993676316 | 6.956575471 | 6.895045455 | | | | |
| MX2 | 7.97713 | 8.098521053 | 8.082936765 | 7.968677273 | | | | |
| CHIC2 | 8.378606667 | 8.498828947 | 8.484154412 | 8.42045 | | | | |
| EIF4E3 | 8.776916667 | 8.931847368 | 8.881707353 | 8.754254545 | | | | |
| RPL37 | 5.6613 | 5.780355263 | 5.765086765 | 5.696131818 | | | | |
| ELF1 | 10.33958333 | 10.43980263 | 10.44328824 | 10.32341818 | | | | |
| BAT2D | 5.830226667 | 5.947547368 | 5.932069118 | 5.837931818 | | | | |
| 4930404F20RIK | 6.658073333 | 6.724336842 | 6.759348529 | 6.646877273 | | | | |
| LOC100044190 | 10.56954667 | 10.72565263 | 10.67076618 | 10.57017273 | | | | |
| EP5TI1 | 8.180176667 | 8.271594737 | 8.281332353 | 8.206504545 | | | | |
| GPI1 | 11.68305667 | 11.81179474 | 11.7829 | 11.72728182 | | | | |
| 4930422G04RIK | 5.690713333 | 5.795713158 | 5.789577941 | 5.735836364 | | | | |
| RBM14 | 6.059196667 | 6.178389474 | 6.157111765 | 6.07645 | | | | |
| 4932413O14RIK | 5.634773333 | 5.714357895 | 5.731648529 | 5.634990909 | | | | |
| CD247 | 6.427023333 | 6.531918421 | 6.523489706 | 6.396290909 | | | | |
| CCR4 | 5.72549 | 5.829228947 | 5.821095588 | 5.752877273 | | | | |
| ZFP800 | 6.687236667 | 6.769447368 | 6.782586765 | 6.685253636 | | | | |
| PABPC1 | 11.55015 | 11.68038684 | 11.64491176 | 11.52837273 | | | | |
| D230021E06RIK | 7.510763333 | 7.665360526 | 7.605245588 | 7.514104545 | | | | |
| SELP | 6.475836667 | 6.566694737 | 6.569254412 | 6.479186364 | | | | |
| TSPAN14 | 9.755656667 | 9.861615789 | 9.848317647 | 9.756059091 | | | | |
| V1RD18 | 5.599066667 | 5.703860526 | 5.691057353 | 5.629445455 | | | | |
| CD226 | 6.903246667 | 6.998228947 | 6.995114706 | 6.884318182 | | | | |
| Mgeneric | 6.73558 | 6.829271053 | 6.826967647 | 6.755563636 | | | | |
| 2310016C16RIK | 5.732096667 | 5.854347368 | 5.823288235 | 5.757786364 | | | | |
| DCK | 6.626113333 | 6.745452632 | 6.716342647 | 6.582109091 | | | | |
| PCDHGC5 | 5.98469 | 6.068434211 | 6.074589706 | 5.985395455 | | | | |
| UBE2Z | 6.080933333 | 6.175294737 | 6.168729412 | 6.086440909 | | | | |
| PLXNC1 | 6.419863333 | 6.511336842 | 6.506238235 | 6.43105 | | | | |
| PSEN2 | 5.908983333 | 5.994313158 | 5.994827941 | 5.915718182 | | | | |
| STAT3 | 10.98784 | 11.10429737 | 11.07339706 | 10.99688182 | | | | |
| LOC628101 | 6.481883333 | 6.588642105 | 6.566361765 | 6.46445 | | | | |
| GREM2 | 5.9519 | 6.052036842 | 6.035844118 | 5.954177273 | | | | |
| INPP4B | 6.231216667 | 6.344565789 | 6.313639706 | 6.238772727 | | | | |
| NEO1 | 6.07765 | 6.151984211 | 6.158933824 | 6.063904545 | | | | |
| 1700027N10RIK | 6.36718 | 6.440963158 | 6.448286765 | 6.3687 | | | | |
| ZFP277 | 9.87083 | 9.932757895 | 9.951282353 | 9.841213636 | | | | |
| GNL3L | 7.609203333 | 7.714147368 | 7.689342647 | 7.616795455 | | | | |
| LOC100047009 | 5.947723333 | 6.043807895 | 6.027170588 | 5.933 | | | | |
| PPP2R5C | 9.96416 | 10.07191842 | 10.04338529 | 9.967009091 | | | | |
| LOC235279 | 5.738703333 | 5.820126316 | 5.817807353 | 5.745463636 | | | | |
| PLEKHF2 | 8.16861 | 8.274002632 | 8.246895588 | 8.169977273 | | | | |
| IL1F10 | 5.98729 | 6.069436842 | 6.065126471 | 5.986545455 | | | | |
| 3202002H23RIK | 6.4492 | 6.538394737 | 6.526935294 | 6.422209091 | | | | |
| LOC381093 | 5.809203333 | 5.883715789 | 5.884635294 | 5.790786364 | | | | |
| KRT25 | 5.72627 | 5.813852632 | 5.800451471 | 5.73225 | | | | |
| VMN2R81 | 5.918906667 | 5.979321053 | 5.992120588 | 5.916390909 | | | | |
| FERD3L | 5.81418 | 5.896818421 | 5.887038235 | 5.798140909 | | | | |
| TELO2 | 5.800226667 | 5.898847368 | 5.872701471 | 5.773190909 | | | | |
| 9430077D24RIK | 7.0186 | 7.135728947 | 7.091011765 | 6.99615 | | | | |
| SLC30A8 | 5.580283333 | 5.643439474 | 5.652214706 | 5.570672727 | | | | |
| TRAPPC2 | 5.7618 | 5.839247368 | 5.832552941 | 5.76225 | | | | |
| C920025C15RIK | 6.85869 | 6.949318421 | 6.929292647 | 6.796427273 | | | | |
| KLRA1 | 5.763913333 | 5.846684211 | 5.833513235 | 5.760622727 | | | | |
| EG386506 | 5.675066667 | 5.7339 | 5.743238235 | 5.663213636 | | | | |
| LOC383306 | 5.85391 | 5.963960526 | 5.920935294 | 5.8559 | | | | |
| VMN2R89 | 6.176576667 | 6.259428947 | 6.242405882 | 6.168013636 | | | | |
| LOC244219 | 5.892636667 | 5.952907895 | 5.958372059 | 5.870513636 | | | | |
| 2610017G09RIK | 5.774383333 | 5.840123684 | 5.839426471 | 5.774468182 | | | | |
| LOC386161 | 5.588033333 | 5.657231579 | 5.651886765 | 5.595286364 | | | | |
| NCDN | 5.806293333 | 5.869863158 | 5.869651471 | 5.803568182 | | | | |
| SLFN9 | 5.536233333 | 5.60855 | 5.597972059 | 5.544027273 | | | | |
| LOC331243 | 5.580336667 | 5.652857895 | 5.641372059 | 5.5795 | | | | |
| RAB33A | 6.031763333 | 6.095615789 | 6.092541176 | 5.987954545 | | | | |
| DTYMK | 10.10785 | 10.17909737 | 10.16820147 | 10.11717727 | | | | |
| LOC100040244 | 7.19639 | 7.280847368 | 7.256533824 | 7.172322727 | | | | |
| OS9 | 9.561276667 | 9.660952632 | 9.621010294 | 9.559304545 | | | | |
| D630011N09RIK | 5.565416667 | 5.621328947 | 5.624910294 | 5.555445455 | | | | |
| A930014D08RIK | 5.644493333 | 5.696589474 | 5.703229412 | 5.633840909 | | | | |
| MYNN | 5.760286667 | 5.832828947 | 5.818648529 | 5.768645455 | | | | |
| SPNB2 | 5.695196667 | 5.750415789 | 5.752666176 | 5.672931818 | | | | |
| RNF19A | 5.905073333 | 5.992334211 | 5.962323529 | 5.879622727 | | | | |
| ARHGAP25 | 10.10459 | 10.18453947 | 10.16155 | 10.08619091 | | | | |
| LOC383433 | 5.61099 | 5.677026316 | 5.666641176 | 5.608054545 | | | | |
| MIER2 | 5.829166667 | 5.889905263 | 5.884575 | 5.805927273 | | | | |
| PDIA2 | 5.79263 | 5.866986842 | 5.846214706 | 5.779913636 | | | | |
| LOC384990 | 5.856243333 | 5.924778947 | 5.908864706 | 5.849868182 | | | | |
| LOC100043391 | 14.24296667 | 14.31736842 | 14.29466324 | 14.21495 | | | | |
| LOC100040854 | 5.568043333 | 5.614126316 | 5.618561765 | 5.542736364 | | | | |
| PIGN | 5.77965 | 5.838668421 | 5.830154412 | 5.7613 | | | | |
| BAZ1B | 5.625716667 | 5.692768421 | 5.676020588 | 5.621545455 | | | | |
| TTLL4 | 5.922753333 | 5.996405263 | 5.973054412 | 5.912381818 | | | | |
| DHX32 | 8.92933 | 9.010481579 | 8.979463235 | 8.9256 | | | | |
| D4ERTD22E | 11.32444333 | 11.39864211 | 11.37389118 | 11.32272727 | | | | |
| LOC100048816 | 5.880436667 | 5.965505263 | 5.928376471 | 5.871918182 | | | | |
| GPC3 | 5.648313333 | 5.711607895 | 5.695651471 | 5.644954545 | | | | |
| LOC384585 | 5.716866667 | 5.787997368 | 5.764117647 | 5.711304545 | | | | |
| OLFR701 | 5.69437 | 5.76715 | 5.738292647 | 5.683054545 | | | | |
| FANCA | 5.56208 | 5.625584211 | 5.605772059 | 5.55515 | | | | |
| DOWN IN GA AND REF ONLY | | | | | | | | |
| LOC386360 | 9.76067 | 9.543934211 | 9.445438235 | 9.669977273 | | | | |
| CLEC4E | 7.003983333 | 6.697260526 | 6.759897059 | 6.9338 | | | | |
| LOC385923 | 7.89017 | 7.690592105 | 7.650747059 | 7.901590909 | Pattern Represented by Data: | | | |
| C5F3R | 6.042593333 | 5.823492105 | 5.813885294 | 5.969613636 | | | | |
| CD33 | 6.606853333 | 6.339052632 | 6.381583824 | 6.556072727 | x | | | x |
| CD63 | 7.70484 | 7.482515789 | 7.484204412 | 7.655954545 | | x | x | |
| CXCR4 | 9.36576 | 9.097465789 | 9.153819118 | 9.316654545 | Medium | Reference | GA | generic |
| AA536749 | 8.959366667 | 8.7437 | 8.750286765 | 8.878977273 | | | | |
| LOC386268 | 6.859343333 | 6.705134211 | 6.655375 | 6.828495455 | | | | |
| genericLS | 6.407056667 | 6.252378947 | 6.211517647 | 6.348690909 | | | | |
| GAS7 | 6.87571 | 6.687760526 | 6.685158824 | 6.846809091 | | | | |
| KLC4 | 7.976776667 | 7.781668421 | 7.786357353 | 7.905563636 | | | | |
| LOC386288 | 7.19565 | 7.004444737 | 7.005695588 | 7.089990909 | | | | |
| TMEM38B | 8.486633333 | 8.312155263 | 8.299532353 | 8.423818182 | | | | |
| GPR97 | 6.329983333 | 6.156044737 | 6.1434 | 6.272113636 | | | | |
| 1100001G20RIK | 6.425286667 | 6.217157895 | 6.238989706 | 6.366827273 | | | | |
| APP | 7.13296 | 6.940878947 | 6.949335294 | 7.052945455 | | | | |
| 4930572J05RIK | 10.59237333 | 10.43747105 | 10.41293676 | 10.54924091 | | | | |
| TGIF1 | 8.40255 | 8.232644737 | 8.224369118 | 8.362318182 | | | | |
| LOC243025 | 6.799403333 | 6.631713158 | 6.622926471 | 6.726436364 | | | | |
| SEC16A | 10.14415 | 10.00321316 | 9.968227941 | 10.07729545 | | | | |
| BC014795 | 8.22279 | 8.047292105 | 8.050194118 | 8.188168182 | | | | |
| CFB | 6.413743333 | 6.207484211 | 6.243664706 | 6.349068182 | | | | |
| TMEM63A | 7.836176667 | 7.698636842 | 7.666680882 | 7.774609091 | | | | |
| DNMT3L | 6.21078 | 6.081707895 | 6.041905882 | 6.181622727 | | | | |
| ACBD4 | 9.321576667 | 9.179684211 | 9.154017647 | 9.266631818 | | | | |
| ORF19 | 7.046476667 | 6.849634211 | 6.879329412 | 6.964309091 | | | | |
| CREG1 | 6.78207 | 6.618634211 | 6.615505882 | 6.743304545 | | | | |
| IRAK3 | 9.18481 | 9.031655263 | 9.019070588 | 9.132704545 | | | | |
| BC065397 | 6.624853333 | 6.452586842 | 6.459883824 | 6.546759091 | | | | |
| ARHGAP22 | 6.35587 | 6.161063158 | 6.192420588 | 6.264263636 | | | | |
| GAA | 8.179186667 | 8.011515789 | 8.021354412 | 8.115145455 | | | | |
| LOC386117 | 7.206693333 | 7.060794737 | 7.051636765 | 7.15335 | | | | |
| S031414D18RIK | 8.621983333 | 8.465123684 | 8.467298529 | 8.571345455 | | | | |
| IRF3 | 7.902646667 | 7.720826316 | 7.748907353 | 7.866604545 | | | | |
| IGLC2_J00595_{—} | 13.84993333 | 13.69477895 | 13.69675147 | 13.85070455 | | | | |
| IG_LAMBDA_{—} | | | | | | | | |
| CONSTANT_2_14 | | | | | | | | |
| BRD2 | 11.40748667 | 11.26870789 | 11.25570735 | 11.342 | | | | |
| SRGAP2 | 7.94865 | 7.758986842 | 7.797158824 | 7.879281818 | | | | |
| ZFP36L1 | 13.96748 | 13.84238684 | 13.81608971 | 13.94897273 | | | | |
| MYO7A | 6.819533333 | 6.64505 | 6.668164706 | 6.745968182 | | | | |
| LOC382163 | 6.892846667 | 6.712444737 | 6.742497059 | 6.811872727 | | | | |
| LOC238836 | 13.98839 | 13.84113421 | 13.83827941 | 13.94409091 | | | | |
| HRSP12 | 8.18713 | 8.021755263 | 8.037048529 | 8.120886364 | | | | |
| GPS2 | 9.44959 | 9.323986842 | 9.303297059 | 9.419718182 | | | | |
| DLG3 | 7.168773333 | 7.001178947 | 7.022592647 | 7.125568182 | | | | |
| 2810402K13RIK | 8.3151 | 8.126965789 | 8.169489706 | 8.250713636 | | | | |
| SHC1 | 11.17267667 | 11.05177105 | 11.02947353 | 11.13645 | | | | |
| GALNT10 | 8.239243333 | 8.110673684 | 8.096067647 | 8.178859091 | | | | |
| IVD | 9.353073333 | 9.195634211 | 9.211955882 | 9.293895455 | | | | |
| RING1 | 8.797403333 | 8.707202632 | 8.656370588 | 8.835413636 | | | | |
| LOC100048105 | 13.31449667 | 13.21344474 | 13.17428971 | 13.29137273 | | | | |
| CLEC4B1 | 6.123163333 | 5.964173684 | 5.98355 | 6.126754545 | | | | |
| TYROBP | 7.546993333 | 7.352484211 | 7.408191176 | 7.491804545 | | | | |
| 6230425C21RIK | 7.159243333 | 6.973836842 | 7.021026471 | 7.173704545 | | | | |
| 2810422O20RIK | 7.155133333 | 7.041215789 | 7.017935294 | 7.145077273 | | | | |
| RFFL | 7.346986667 | 7.233876316 | 7.211079412 | 7.344081818 | | | | |
| CEPT1 | 10.36051667 | 10.25173158 | 10.22485441 | 10.31255909 | | | | |
| NEURL | 6.091016667 | 5.940726316 | 5.956220588 | 6.018363636 | | | | |
| ZFP317 | 7.35243 | 7.224565789 | 7.217720588 | 7.297 | | | | |
| SRGAP3 | 6.20861 | 6.119747368 | 6.074486765 | 6.187718182 | | | | |
| RARG | 6.253086667 | 6.097884211 | 6.120816176 | 6.186990909 | | | | |
| PI4KB | 9.675126667 | 9.524628947 | 9.544104412 | 9.670277273 | | | | |
| GALGT1 | 7.578576667 | 7.461855263 | 7.448160294 | 7.576309091 | | | | |
| PIK3R6 | 6.473133333 | 6.331334211 | 6.342902941 | 6.471927273 | | | | |
| MREG | 9.009966667 | 8.899078947 | 8.880347059 | 9.012027273 | | | | |
| 1200002N14RIK | 7.02541 | 6.885213158 | 6.896238235 | 7.071472727 | | | | |
| 2610028H07RIK | 10.22659 | 10.05706053 | 10.09811029 | 10.20060909 | | | | |
| TGFBI | 6.317396667 | 6.125718421 | 6.189858824 | 6.294936364 | | | | |
| NOS3 | 5.930473333 | 5.810139474 | 5.804051471 | 5.8815 | | | | |
| USP11 | 6.548856667 | 6.408394737 | 6.422813235 | 6.516695455 | | | | |
| FOS | 6.999393333 | 6.903897368 | 6.874805882 | 7.035045455 | | | | |
| LOC636818 | 6.108246667 | 5.997428947 | 5.985791176 | 6.062581818 | | | | |
| SCL0004023.1_57 | 6.11199 | 5.997631579 | 5.989891176 | 6.097618182 | | | | |
| P2RX4 | 7.48024 | 7.348384211 | 7.358327941 | 7.436886364 | | | | |
| DGKQ | 7.016946667 | 6.876660526 | 6.895120588 | 6.966136364 | | | | |
| SNX15 | 9.416226667 | 9.335971053 | 9.294445588 | 9.462340909 | | | | |
| SLC11A1 | 6.549723333 | 6.422 | 6.429011765 | 6.614281818 | | | | |
| TLR13 | 6.13844 | 5.968510526 | 6.018579412 | 6.089945455 | | | | |
| MKL1 | 6.422093333 | 6.290807895 | 6.302858824 | 6.376204545 | | | | |
| EG633640 | 6.2549 | 6.167321053 | 6.135855882 | 6.224536364 | | | | |
| RASGRF1 | 5.882463333 | 5.754576316 | 5.763907353 | 5.8296 | | | | |
| TRP53 | 9.81586 | 9.688071053 | 9.697814706 | 9.816331818 | | | | |
| 1300010F03R1K | 6.72106 | 6.618107895 | 6.603588235 | 6.719240909 | | | | |
| RNF123 | 7.61357 | 7.475247368 | 7.4966 | 7.591468182 | | | | |
| LOC386085 | 6.32579 | 6.223992105 | 6.209532353 | 6.326190909 | | | | |
| TIMM8A2 | 5.952686667 | 5.835228947 | 5.837423529 | 5.907509091 | | | | |
| CLEC4A1 | 6.377756667 | 6.218557895 | 6.262733824 | 6.464059091 | | | | |
| SGPL1 | 7.595083333 | 7.473997368 | 7.480830882 | 7.646777273 | | | | |
| TMUB1 | 8.66402 | 8.519415789 | 8.549851471 | 8.647472727 | | | | |
| LOC383099 | 13.25080333 | 13.11434737 | 13.13688529 | 13.20990455 | | | | |
| HMOX1 | 6.674133333 | 6.506121053 | 6.561204412 | 6.718672727 | | | | |
| ERCC4 | 8.262506667 | 8.1301 | 8.150180882 | 8.232518182 | | | | |
| SOLH | 8.064173333 | 7.913865789 | 7.952263235 | 8.027909091 | | | | |
| PREP | 8.029716667 | 7.916678947 | 7.918044118 | 7.973690909 | | | | |
| LOC100048105 | 13.58104 | 13.50723421 | 13.47030735 | 13.56905 | | | | |
| GSTM2 | 6.0557 | 5.898139474 | 5.945163235 | 6.033840909 | | | | |
| EPB4.1L3 | 6.005856667 | 5.903615789 | 5.895541176 | 6.006977273 | | | | |
| USP22 | 9.1675 | 9.065165789 | 9.057348529 | 9.184159091 | | | | |
| 1700073E17RIK | 5.778756667 | 5.660986842 | 5.668639706 | 5.740490909 | | | | |
| 1110014N23RIK | 9.78732 | 9.676873684 | 9.677741176 | 9.754181818 | | | | |
| RCCD1 | 8.2828 | 8.183665789 | 8.173695588 | 8.285281818 | | | | |
| ANKRD13A | 10.35183 | 10.25657632 | 10.24345 | 10.33284091 | | | | |
| SP2 | 6.97502 | 6.871657895 | 6.866919118 | 6.955763636 | | | | |
| CENTG3 | 6.891463333 | 6.743584211 | 6.784157353 | 6.877472727 | | | | |
| 3110004L20RIK | 6.040683333 | 5.938431579 | 5.933769118 | 6.049186364 | | | | |
| 6430526N21RIK | 7.338353333 | 7.170902632 | 7.231635294 | 7.327745455 | | | | |
| 2310067B10RIK | 7.60857 | 7.454689474 | 7.501908824 | 7.593022727 | | | | |
| 1810030N24RIK | 6.94621 | 6.833397368 | 6.840070588 | 6.905022727 | | | | |
| RBM15 | 8.00761 | 7.891310526 | 7.901594118 | 7.997504545 | | | | |
| WWP2 | 8.939 | 8.862852632 | 8.833398529 | 8.9579 | | | | |
| 2610024M03RIK | 6.12332 | 6.027731579 | 6.018229412 | 6.085745455 | | | | |
| C430003P19RIK | 9.67627 | 9.606163158 | 9.571339706 | 9.665418182 | | | | |
| BC066107 | 6.6825 | 6.565544737 | 6.577964706 | 6.707777273 | | | | |
| 3110082D06RIK | 6.0938 | 5.993510526 | 5.989636765 | 6.068118182 | | | | |
| LYNX1 | 8.635493333 | 8.522728947 | 8.531370588 | 8.607563636 | | | | |
| PRKCZ | 7.069753333 | 6.920815789 | 6.966716176 | 7.081818182 | | | | |
| IGK-V33 | 10.09401333 | 9.976852632 | 9.992255882 | 10.10707727 | | | | |
| SLC11A1 | 6.997713333 | 6.838144737 | 6.896435294 | 7.107336364 | | | | |
| CBLC | 5.966836667 | 5.825323684 | 5.865586765 | 5.941622727 | | | | |
| B930049N04RIK | 5.693346667 | 5.604668421 | 5.592219118 | 5.679722727 | | | | |
| CASP1 | 11.61187333 | 11.54446316 | 11.51241912 | 11.61258182 | | | | |
| MBD1 | 9.107946667 | 8.991671053 | 9.009091176 | 9.104772727 | | | | |
| 2510022D24RIK | 8.239006667 | 8.136547368 | 8.141229412 | 8.224509091 | | | | |
| FRMPD1 | 5.682146667 | 5.613552632 | 5.584472059 | 5.669695455 | | | | |
| USP54 | 7.069403333 | 6.990452632 | 6.971775 | 7.068827273 | | | | |
| FA2H | 6.074253333 | 5.976113158 | 5.976633824 | 6.047031818 | | | | |
| EG434402 | 9.268486657 | 9.145802632 | 9.171463235 | 9.227836364 | | | | |
| SLC9A4 | 5.812063333 | 5.718157895 | 5.715114706 | 5.784231818 | | | | |
| ATP6V1B2 | 10.49800333 | 10.43401842 | 10.40123971 | 10.54807273 | | | | |
| 2310079N02RIK | 9.192623333 | 9.095097368 | 9.097661765 | 9.17595 | | | | |
| ZFPL1 | 7.48469 | 7.385313158 | 7.390122059 | 7.521240909 | | | | |
| NISCH | 7.18114 | 7.045213158 | 7.087505882 | 7.181422727 | | | | |
| FCMD | 6.566743333 | 6.486473684 | 6.473394118 | 6.569354545 | | | | |
| 1810009N02RIK | 8.202653333 | 8.123931579 | 8.109642647 | 8.261063636 | | | | |
| 6720486H14RIK | 5.675496667 | 5.599039474 | 5.583961765 | 5.670122727 | | | | |
| AW011738 | 6.340756667 | 6.277447368 | 6.2494 | 6.329786364 | | | | |
| COgeneric | 8.195383333 | 8.085518421 | 8.104542647 | 8.177086364 | | | | |
| PCBP2 | 12.72348667 | 12.62733947 | 12.63373235 | 12.69256364 | | | | |
| C330019G07RIK | 6.827063333 | 6.742765789 | 6.738351471 | 6.829163636 | | | | |
| SMARCE1 | 9.420046667 | 9.316405263 | 9.331558824 | 9.392968182 | | | | |
| ABCC4 | 7.844656667 | 7.752781579 | 7.756655882 | 7.838990909 | | | | |
| GTPBP1 | 9.273093333 | 9.164928947 | 9.185194118 | 9.281127273 | | | | |
| SLC25A42 | 6.299283333 | 6.193821053 | 6.212104412 | 6.306677273 | | | | |
| 5830415F09RIK | 7.08473 | 6.966402632 | 6.997625 | 7.052472727 | | | | |
| PEO1 | 7.222113333 | 7.107710526 | 7.1356 | 7.205309091 | | | | |
| CRTAC1 | 6.011773333 | 5.950531579 | 5.925355882 | 6.037995455 | | | | |
| CLEC5A | 5.95908 | 5.828018421 | 5.873202941 | 5.958354545 | | | | |
| ZFP704 | 5.9147 | 5.849613158 | 5.828823529 | 5.941309091 | | | | |
| TNS4 | 5.844593333 | 5.780189474 | 5.758717647 | 5.866986364 | | | | |
| 2310040G07RIK | 5.79773 | 5.696360526 | 5.712151471 | 5.798627273 | | | | |
| KRTAP5-4 | 5.736486667 | 5.664107895 | 5.651308824 | 5.727004545 | | | | |
| D030051N19RIK | 8.371213333 | 8.285613158 | 8.287195588 | 8.383390909 | | | | |
| A430043M19RIK | 6.192146667 | 6.116665789 | 6.108377941 | 6.186131818 | | | | |
| 9030025P20RIK | 8.676186667 | 8.607128947 | 8.592552941 | 8.680018182 | | | | |
| GAS7 | 6.734093333 | 6.607315789 | 6.6505 | 6.764486364 | | | | |
| TBC1D25 | 8.04817 | 7.934818421 | 7.965151471 | 8.058922727 | | | | |
| LOC193563 | 5.841313333 | 5.780481579 | 5.758816176 | 5.863127273 | | | | |
| PAPSS2 | 6.744143333 | 6.628768421 | 6.661655882 | 6.768309091 | | | | |
| ELL | 9.71715 | 9.633271053 | 9.634791176 | 9.697881818 | | | | |
| SHKBP1 | 8.369346667 | 8.246628947 | 8.287191176 | 8.407618182 | | | | |
| RNF10 | 11.53589 | 11.45141053 | 11.45382206 | 11.53415455 | | | | |
| FBXL20 | 7.886646667 | 7.80505 | 7.804917647 | 7.885722727 | | | | |
| TEAD4 | 6.07582 | 6.003681579 | 5.99445 | 6.073868182 | | | | |
| 5031439G07RIK | 8.046333333 | 7.975907895 | 7.966855882 | 8.056904545 | | | | |
| LOC100046003 | 8.814313333 | 8.735331579 | 8.734867647 | 8.801768182 | | | | |
| B130002B06RIK | 5.813043333 | 5.732515789 | 5.7347 | 5.7991 | | | | |
| EPN3 | 5.787266667 | 5.702681579 | 5.710923529 | 5.770054545 | | | | |
| ZC3H12A | 8.291086667 | 8.213755263 | 8.215352941 | 8.354177273 | | | | |
| 2900079J23RIK | 5.841546667 | 5.736136842 | 5.765825 | 5.837059091 | | | | |
| CLEC4B1 | 5.798906667 | 5.716681579 | 5.724957353 | 5.853954545 | | | | |
| 1300010F03RIK | 6.837643333 | 6.766381579 | 6.763723529 | 6.82175 | | | | |
| KRT39 | 5.62461 | 5.520444737 | 5.551114706 | 5.6101 | | | | |
| 4930519F16RIK | 5.689996667 | 5.611807895 | 5.616554412 | 5.702931818 | | | | |
| RHBDD1 | 7.70038 | 7.587589474 | 7.627182353 | 7.714345455 | | | | |
| GDPD1 | 6.465226667 | 6.393168421 | 6.392429412 | 6.466463636 | | | | |
| ACP2 | 6.88359 | 6.791065789 | 6.810866176 | 6.8907 | | | | |
| SETD1A | 9.028223333 | 8.94825 | 8.955577941 | 9.065645455 | | | | |
| OLFR516 | 5.832843333 | 5.755394737 | 5.760235294 | 5.846913636 | | | | |
| REN1 | 5.670936667 | 5.596518421 | 5.598602941 | 5.650036364 | | | | |
| 5330417C22RIK | 5.710553333 | 5.612434211 | 5.640252941 | 5.719795455 | | | | |
| AGAP1 | 6.059153333 | 5.948686842 | 5.989294118 | 6.109681818 | | | | |
| DUSP6 | 5.8376 | 5.760176316 | 5.768070588 | 5.856759091 | | | | |
| CTSL | 5.81264 | 5.718744737 | 5.743730882 | 5.827822727 | | | | |
| SLCO6C1 | 5.686233333 | 5.602471053 | 5.617510294 | 5.693277273 | | | | |
| TBC1D2 | 6.907116667 | 6.793013158 | 6.838802941 | 6.992163636 | | | | |
| OPHN1 | 5.778693333 | 5.693957895 | 5.710407353 | 5.780409091 | | | | |
| B930048N13RIK | 5.961773333 | 5.883607895 | 5.894567647 | 5.95935 | | | | |
| MBL1 | 5.619243333 | 5.55345 | 5.552047059 | 5.635831818 | | | | |
| A730017D01RIK | 6.58584 | 6.519621053 | 6.518897059 | 6.621677273 | | | | |
| SNX33 | 7.0541 | 6.931763158 | 6.987372059 | 7.065340909 | | | | |
| E330023P07RIK | 5.836216667 | 5.778460526 | 5.770314706 | 5.832118182 | | | | |
| 9330152L17 | 5.81598 | 5.740573684 | 5.750429412 | 5.823040909 | | | | |
| PCDH19 | 5.694973333 | 5.629565789 | 5.630432353 | 5.695972727 | | | | |
| CAGE1 | 5.926 | 5.816742105 | 5.861688235 | 5.935345455 | | | | |
| TMEM56 | 8.612496667 | 5.559673684 | 5.548541176 | 5.640304545 | | | | |
| ACTR3B | 6.533076667 | 6.4662 | 6.469547059 | 6.5374 | | | | |
| ADAM11 | 5.691363333 | 5.605971053 | 5.628829412 | 5.689736364 | | | | |
| CD151 | 8.090336667 | 8.015257895 | 8.028898529 | 8.088440909 | | | | |
| A930014P08RIK | 5.6155 | 5.555547368 | 5.554239706 | 5.521622727 | | | | |
| SLC35E4 | 5.867336667 | 5.791586842 | 5.806241176 | 5.91005 | | | | |
| PARD3B | 5.96533 | 5.880542105 | 5.904663235 | 5.975636364 | | | | |
| OLFR586 | 5.601076667 | 5.522747368 | 5.541236765 | 5.627940909 | | | | |
| D030072B18RIK | 5.61957 | 5.547055263 | 5.559957353 | 5.631459091 | | | | |
| STARD3 | 8.494313333 | 8.422068421 | 8.435257353 | 8.509754545 | | | | |
| SFXN3 | 6.08399 | 6.006510526 | 6.025077941 | 6.121263636 | | | | |
| 6430511F03 | 6.304863333 | 6.2371 | 6.246623529 | 6.322872727 | | | | |
| PROM | 5.928333333 | 5.855444737 | 5.870188235 | 5.950177273 | | | | |
| NTE | 8.366106667 | 8.27475 | 8.3088 | 8.373113636 | | | | |
| OLFR150 | 5.682813333 | 5.620110526 | 5.625554412 | 5.675772727 | | | | |
| 2010001M09RIK | 13.25162667 | 13.17815526 | 13.19607647 | 13.31843636 | | | | |
| KCNC4 | 5.88774 | 5.814560526 | 5.832483824 | 5.911813636 | | | | |
| GM498 | 5.727336667 | 5.681271053 | 5.673501471 | 5.744981818 | | | | |
| LOC327767 | 6.031383333 | 5.952915789 | 5.978073529 | 6.061031818 | | | | |
| B930097H17RIK | 5.844733333 | 5.755813158 | 5.792060294 | 5.865090909 | | | | |
| BMP3 | 5.675396667 | 5.620692105 | 5.622867647 | 5.694827273 | | | | |
| PLEKHB2 | 9.05776 | 8.97335 | 9.006016176 | 9.055263636 | | | | |
| A630038P11RIK | 5.602433333 | 5.537278947 | 5.551530882 | 5.614654545 | | | | |
| 6720480F11RIK | 5.689046667 | 5.629671053 | 5.641458824 | 5.696195455 | | | | |
| CNTNAP3 | 5.841703333 | 5.786078947 | 5.796410294 | 5.878831818 | | | | |

To further examine the impact of each medicine on specific immune system cells, we generated a list of genes with high cell-type specificity for Tregs, and utilized GSEA to determine the extent to which these Treg-specific genes were overrepresented at the top of the ranked list of genes differentially expressed between the two medicines. We confirmed that these Treg-specific genes were overrepresented at the top of the ranked list with higher expression in GA-activated samples than PG-activated samples (FDR-adjusted q=0.00, FIG. 7B).

Taken together, these findings emphasize that GA upregulates FoxP3+ Tregs more consistently and to a higher level than PG. This finding has implications for efficacy.

### EXAMPLE 9

Comparing Potential Safety-Related Impacts on Key Immune System Cell Types: PG May Upregulate Myeloid Lineage Cells to a Greater Extent than GA

Using the ANOVA-based pattern analysis (Methods), we identified a list of genes that are significantly upregulated only by PG compared to medium, and not by GA or GA reference standard compared to medium

(Table 11). Cell type enrichment yielded a variety of stromal cells, macrophages, and monocytes (Table 11). Similarly, genes that were significantly downregulated by GA and GA reference standard relative to medium, but not by PG relative to medium (Table 10) were most enriched for macrophages, monocytes, and granulocytes (Table 11). Finally, genes that have significantly higher expression in samples activated by PG than in samples activated by GA by 4 different parametric methods (Table 8) were enriched primarily in macrophages and monocytes (Table 11).

TABLE 11
Outputs of cell-type enrichment analyses for various lists of genes.

| | 4-method_up GA | | adjusted | | 4-method_up generic | | adjusted |
|---|---|---|---|---|---|---|---|
| Sample ID | Score | p-value | p-value | Sample ID | Score | p-value | p-value |
| T.8Nve.Sp.OT1.rnk | 78.0167172 | 1.30E−93 | 3.14E−92 | MF.Lu.rnk | 52.3659832 | 4.74E−19 | 5.33E−18 |
| T.4FP3.25..LN.rnk | 77.2585738 | 4.58E−92 | 9.88E−91 | MF.BM.rnk | 52.1676734 | 8.50E−27 | 4.12E−25 |
| CD4Control.rnk | 74.7924284 | 5.10E−82 | 4.95E−81 | Mo.6C.II..BM.rnk | 51.5668819 | 3.63E−25 | 1.41E−23 |
| CD4TESTCJ.rnk | 74.3049572 | 7.18E−100 | 3.48E−98 | SC.GMP.BM.rnk | 51.5587442 | 7.77E−33 | 7.53E−31 |
| T.4Nve.Sp.rnk | 74.1176116 | 6.71E−104 | 6.51E−102 | MF.11cloSer.SI.rnk | 46.9677757 | 2.67E−16 | 1.56E−15 |
| T.8SP24..Th.rnk | 73.1331042 | 6.90E−91 | 1.34E−89 | MLP.FL.rnk | 46.519979 | 7.74E−32 | 5.01E−30 |
| T.8Nve.LN.rnk | 72.6081647 | 2.84E−97 | 7.86E−96 | MF.Thio5.II.480int.PC.rnk | 46.0938609 | 1.84E−18 | 1.55E−17 |
| T.4SP24..Th.rnk | 70.7668281 | 3.66E−98 | 1.18E−96 | MF.Thio5.II.480hi.PC.rnk | 45.2029355 | 2.74E−16 | 1.56E−15 |
| T.8Nve.MLN.rnk | 69.029666 | 2.66E−98 | 1.03E−96 | MF.103.11b.SI.rnk | 45.1219289 | 6.17E−10 | 1.93E−09 |
| T.8SP24int.Th.rnk | 64.9653532 | 5.23E−76 | 4.06E−75 | MF.II.480hi.PC.rnk | 42.2591115 | 5.29E−17 | 3.66E−16 |
| T.4Nve.LN.rnk | 63.6821853 | 1.43E−100 | 9.23E−99 | MF.11cloSer.Salm3.SI.rnk | 40.7916014 | 2.09E−15 | 1.04E−14 |
| CD4TESTJS.rnk | 60.5711373 | 1.45E−90 | 2.56E−89 | MF.II.480lo.PC.rnk | 39.3484707 | 1.11E−20 | 1.66E−19 |
| Tgd.vg2..Sp.rnk | 59.521782 | 1.14E−81 | 1.05E−80 | MF.103.11b..Salm3.SI.rnk | 37.9163065 | 4.52E−12 | 1.79E−11 |
| T.4Mem.Sp.rnk | 58.4053995 | 1.28E−74 | 9.20E−74 | MF.Thio5.II.480lo.PC.rnk | 37.2951887 | 6.98E−16 | 3.87E−15 |
| T.4Mem44h62l.Sp.rnk | 57.6060554 | 1.08E−73 | 6.98E−73 | DC.8..Th.rnk | 36.1076603 | 1.28E−16 | 8.46E−16 |

| | 4-method_up GA | | adjusted | | 4-method_up generic | | adjusted |
|---|---|---|---|---|---|---|---|
| Cell type | Score | p-value | p-value | Cell type | Score | p-value | p-value |
| CD8+ T-cell | 78.0167172 | 1.30E−93 | 3.14E−92 | Macrophage Cell | 52.3659832 | 4.74E−19 | 5.33E−18 |
| ***CD4+ T-cell | 77.2585738 | 4.58E−92 | 9.88E−91 | Macrophage Cell | 52.1676734 | 8.50E−27 | 4.12E−25 |
| (FOXP3+)*** | | | | | | | |
| CD4+ T-cell | 74.7924284 | 5.10E−82 | 4.95E−81 | ***MONOCYTE CELL*** | 51.5668819 | 3.63E−25 | 1.41E−23 |
| CD4+ T-cell | 74.3049572 | 7.18E−100 | 3.48E−98 | Stromal Cell | 51.5587442 | 7.77E−33 | 7.53E−31 |
| CD4+ T-cell | 74.1176116 | 6.71E−104 | 6.51E−102 | Macrophage Cell | 46.9677757 | 2.67E−16 | 1.56E−15 |
| CD8+ T-cell | 73.1331042 | 6.90E−91 | 1.34E−89 | Multi-lineage Progenitor | 46.519979 | 7.74E−32 | 5.01E−30 |
| CD8+ T-cell | 72.6081647 | 2.84E−97 | 7.86E−96 | Macrophage Cell | 46.0938609 | 1.84E−18 | 1.55E−17 |
| CD4+ T-cell | 70.7668281 | 3.66E−98 | 1.18E−96 | Macrophage Cell | 45.2029355 | 2.74E−16 | 1.56E−15 |
| CD8+ T-cell | 69.029666 | 2.66E−98 | 1.03E−96 | Macrophage Cell | 45.1219289 | 6.17E−10 | 1.93E−09 |
| CD8+ T-cell | 64.9653532 | 5.23E−76 | 4.06E−75 | Macrophage Cell | 42.2591115 | 5.29E−17 | 3.66E−16 |
| CD4+ T-cell | 63.6821853 | 1.43E−100 | 9.23E−99 | Macrophage Cell | 40.7916014 | 2.09E−15 | 1.04E−14 |
| CD4+ T-cell | 60.5711373 | 1.45E−90 | 2.56E−89 | Macrophage Cell | 39.3484707 | 1.11E−20 | 1.66E−19 |
| Gamma Delta T-Cell | 59.521782 | 1.14E−81 | 1.05E−80 | Macrophage Cell | 37.9163065 | 4.52E−12 | 1.79E−11 |
| CD4+ T-cell | 58.4053995 | 1.28E−74 | 9.20E−74 | Macrophage Cell | 37.2951887 | 6.98E−16 | 3.87E−15 |
| CD4+ T-cell | 57.6060554 | 1.08E−73 | 6.98E−73 | Dendritic Cell | 36.1076603 | 1.28E−16 | 8.46E−16 |

| | UP_in_GA | | adjusted | | DOWN_in_GA | | adjusted |
|---|---|---|---|---|---|---|---|
| Sample ID | Score | p-value | p-value | Sample ID | Score | p-value | p-value |
| Tgd.vg2.24ahi.e17.Th.rnk | 55.3001141 | 9.65E−11 | 2.27E−09 | MF.Thio5.II.480int.PC.rnk | 78.3629937 | 7.87E−13 | 6.06E−11 |
| Tgd.vg1.vd6.24ahi.Th.rnk | 51.6590126 | 1.41E−11 | 5.31E−10 | MF.103.11b..SI.rnk | 70.6792182 | 1.05E−05 | 5.38E−05 |
| T.8Eff.Sp.OT1.d5.VSVOva.rnk | 49.3859076 | 3.58E−15 | 3.37E−13 | MF.11cloSer.SI.rnk | 70.0561042 | 8.81E−09 | 6.46E−08 |
| Tgd.vg3.24alo.e17.Th.rnk | 48.4433864 | 1.36E−10 | 2.57E−09 | MF.Thio5.II.480hi.PC.rnk | 69.920643 | 4.45E−10 | 7.61E−09 |
| T.8Eff.Sp.OT1.d6.LisOva.rnk | 44.7876477 | 5.19E−11 | 1.39E−09 | MF.11cloSer.Salm3.SI.rnk | 66.6852735 | 3.26E−12 | 1.25E−10 |
| NKT.44.NK1.1..Th.rnk | 44.3537644 | 5.06E−07 | 3.07E−06 | MF.103.11b..Salm3.SI.rnk | 62.9428165 | 1.69E−09 | 1.63E−08 |
| T.8SP24int.Th.rnk | 42.865925 | 4.45E−08 | 4.92E−07 | GN.BM.rnk | 60.9658676 | 3.15E−10 | 6.06E−09 |
| T.DPbl.Th.rnk | 42.1959412 | 7.68E−10 | 1.31E−08 | MF.Lu.rnk | 58.3863313 | 8.60E−11 | 2.21E−09 |
| Tgd.vg5.24ahi.Th.rnk | 40.9919756 | 3.97E−12 | 1.86E−10 | MF.II.480hi.PC.rnk | 55.6976908 | 9.43E−10 | 1.25E−08 |
| T.8Eff.Sp.OT1.48hr.LisOva.rnk | 40.8339989 | 8.39E−07 | 4.78E−06 | GN.Thio.PC.rnk | 53.404852 | 8.61E−11 | 2.21E−09 |
| T.4FP3.25..LN.rnk | 40.0931912 | 1.51E−05 | 5.06E−05 | GN.Arth.BM.rnk | 53.1486978 | 1.07E−09 | 1.26E−08 |
| T.4Mem.Sp.rnk | 40.034017 | 6.10E−06 | 2.39E−05 | MF.RP.Sp.rnk | 50.2823835 | 1.63E−09 | 1.63E−08 |
| T.8SP24..Th.rnk | 39.3976068 | 1.32E−07 | 1.25E−06 | MF.Thio5.II.480lo.PC.rnk | 50.1672897 | 6.62E−09 | 5.10E−08 |
| T.4SP24..Th.rnk | 37.6453635 | 1.91E−09 | 2.56E−08 | Mo.6C.II..LN.rnk | 49.3947395 | 9.73E−10 | 1.25E−08 |
| T.4Mem44h62l.Sp.rnk | 36.1232657 | 2.52E−06 | 1.21E−05 | Mo.6C.II..BM.rnk | 49.2230681 | 1.60E−08 | 1.07E−07 |

| | UP_in_GA | | adjusted | | DOWN_in_GA | | adjusted |
|---|---|---|---|---|---|---|---|
| | Score | p-value | p-value | | Score | p-value | p-value |
| Gamma Delta T-Cell | 55.3001141 | 9.6483E−11 | 2.2674E−09 | Macrophage | 78.3629337 | 7.8734E−13 | 6.0625E−11 |
| Gamma Delta T-Cell | 51.6590126 | 1.4116E−11 | 5.3077E−10 | Macrophage | 70.6792182 | 1.0481E−05 | 5.3801E−05 |
| CD8+ T-cell | 49.3859076 | 3.5806E−15 | 3.3658E−13 | Macrophage | 70.0561042 | 8.8126E−09 | 6.4626E−08 |
| Gamma Delta T-Cell | 48.4433864 | 1.3645E−10 | 2.5653E−09 | Macrophage | 69.920643 | 4.4492E−10 | 7.613E−09 |
| CD8+ T-cell | 44.7876477 | 5.1879E−11 | 1.3933E−09 | Macrophage | 66.6852735 | 3.2569E−12 | 1.2539E−10 |
| Natural Killer T-Cell | 44.3537644 | 5.056E−07 | 3.0662E−06 | Macrophage | 62.9428165 | 1.6887E−09 | 1.6254E−08 |
| CD8+ T-cell | 42.865925 | 4.4494E−08 | 4.9205E−07 | Granulocytes | 60.9658676 | 3.1455E−10 | 6.055E−09 |
| CD4+ CD8+ T-cell blast | 42.1959412 | 7.6762E−10 | 1.3119E−08 | Macrophage | 58.3863313 | 8.6018E−11 | 2.2088E−09 |
| Gamma Delta T-Cell | 40.9919756 | 3.967E−12 | 1.8645E−10 | Macrophage | 55.6976908 | 9.4261E−10 | 1.2492E−08 |
| CD8+ T-cell | 40.8339989 | 8.3853E−07 | 4.7771E−06 | Granulocytes | 53.404852 | 8.6056E−11 | 2.2088E−09 |
| ***CD4+ T-cell | 40.0931912 | 1.5074E−05 | 5.0605E−05 | Granulocytes | 53.1486978 | 1.0674E−09 | 1.2645E−08 |
| (FOXP3+)*** | | | | | | | |
| CD4+ T-cell | 40.034017 | 6.104E−06 | 2.3907E−05 | Macrophage | 50.2823835 | 1.6276E−09 | 1.6254E−08 |
| CD8+ T-cell | 39.3976068 | 1.3248E−07 | 1.2453E−06 | Macrophage | 50.1672897 | 6.6206E−09 | 5.0979E−08 |
| CD4+ T-cell | 37.6453635 | 1.9101E−09 | 2.565E−08 | ***MONOCYTE | 49.3947395 | 9.7339E−10 | 1.2492E−08 |
| | | | | CELL*** | | | |
| CD4+ T-cell | 36.1232657 | 2.5163E−06 | 1.213E−05 | ***MONOCYTE | 49.2230681 | 1.6009E−08 | 1.0719E−07 |
| | | | | CELL*** | | | |

| | UP_in_generic | | adjusted | | DOWN_in_generic | | adjusted |
|---|---|---|---|---|---|---|---|
| Sample ID | Score | p-value | p-value | Sample ID | Score | p-value | p-value |
| FRC.MLN.rnk | 52.3529309 | 3.50E−12 | 1.89E−10 | CD4Control.rnk | 27.1284452 | 2.16E−11 | 8.40E−10 |
| FRC.SLN.rnk | 50.8630857 | 4.71E−10 | 9.54E−09 | T.4SP24..Th.rnk | 25.7039936 | 2.33E−10 | 3.70E−09 |
| Fi.Sk.rnk | 50.4164564 | 5.96E−11 | 1.61E−09 | CD4TESTCJ.rnk | 25.0483208 | 4.40E−11 | 1.22E−09 |
| LEC.SLN.rnk | 50.3428482 | 8.82E−07 | 6.81E−06 | T.8Nve.Sp.OT1.rnk | 23.8378135 | 6.69E−10 | 7.63E−09 |
| MF.Thio5.II.480hi.PC.rnk | 46.6144241 | 2.72E−12 | 1.89E−10 | T.4FP3.25..LN.rnk | 23.8306189 | 3.47E−09 | 2.59E−08 |
| LEC.MLN.rnk | 43.3239454 | 4.18E−07 | 3.57E−06 | T.4Nve.Sp.rnk | 23.5977064 | 3.09E−10 | 4.28E−09 |
| St.31.38.44..SLN.rnk | 41.8497581 | 1.77E−08 | 2.60E−07 | T.4SP69..Th.rnk | 22.6133145 | 4.58E−13 | 2.22E−11 |
| BEC.SLN.rnk | 39.0754963 | 5.58E−06 | 3.62E−05 | T.8SP24int.Th.rnk | 22.2048891 | 2.66E−08 | 1.47E−07 |
| BEC.MLN.rnk | 38.2587882 | 1.37E−05 | 8.55E−05 | T.8SP24..Th.rnk | 21.8317119 | 1.02E−08 | 6.17E−06 |
| MF.Thio5.II.480int.PC.rnk | 38.1519547 | 1.13E−11 | 4.56E−10 | T.4SP24int.Th.rnk | 21.5886147 | 7.01E−14 | 6.80E−12 |
| Fi.MTS15..Th.rnk | 34.64529 | 4.61E−05 | 2.34E−04 | T.8Eff.Sp.OT1.d6.LisOva.rnk | 20.6215407 | 9.85E−07 | 3.61E−06 |
| MF.BM.rnk | 32.9364965 | 6.48E−08 | 7.50E−07 | Tgd.vg2.24ahi.e17.Th.rnk | 20.3901357 | 6.79E−04 | 0.0016877 |
| MF.103.11b..SI.rnk | 32.8779969 | 1.54E−05 | 9.22E−05 | T.4Mem44h62l.LN.rnk | 20.1582351 | 4.92E−O9 | 3.41E−08 |
| MF.II.480lo.PC.rnk | 31.9112677 | 9.99E−09 | 1.62E−07 | T.8Nve.LN.rnk | 19.5000548 | 8.43E−10 | 9.09E−09 |
| MF.II.480hi.PC.rnk | 31.7857177 | 6.25E−07 | 5.06E−06 | T.4Mem.Sp.rnk | 19.2816021 | 1.35E−09 | 1.25E−08 |

| | UP_in_generic | | adjusted | | DOWN_in_generic | | adjusted |
|---|---|---|---|---|---|---|---|
| | Score | p-value | p-value | | Score | p-value | p-value |
| fibroblastic reticular cell | 52.3529309 | 3.4974E−12 | 1.8886E−10 | CD4+ T-cell | 27.1284452 | 2.16E−11 | 8.40E−10 |
| fibroblastic reticular cell | 50.8630857 | 4.7108E−10 | 9.5394E−09 | CD4+ T-cell | 25.7039936 | 2.33E−10 | 3.70E−09 |
| fibroblast | 50.4164564 | 5.9643E−11 | 1.6104E−09 | CD4+ T-cell | 25.0483208 | 4.40E−11 | 1.22E−09 |
| lymphatic endothelial cell | 50.3428482 | 8.823E−07 | 6.8063E−06 | CD8+ T-cell | 23.8378135 | 6.69E−10 | 7.63E−09 |
| Macrophages | 46.6144241 | 2.72E−12 | 1.89E−10 | ***CD4+ T-cell | 23.8306189 | 3.4675E−09 | 2.5873E−08 |
| | | | | (FOXP3+)*** | | | |
| lymphatic endothelial cell | 43.3239454 | 4.1832E−07 | 3.5667E−06 | CD4+ T-cell | 23.5977064 | 3.09E−10 | 4.28E−09 |
| Stromal Cell | 41.8497581 | 1.7724E−08 | 2.6032E−07 | CD4+ T-cell | 22.6133145 | 4.58E−13 | 2.22E−11 |
| blood endothelial cell | 39.0754963 | 5.5806E−06 | 3.6162E−05 | CD8+ T-cell | 22.2048891 | 2.66E−08 | 1.47E−07 |
| blood endothelial cell | 38.2587882 | 1.3722E−05 | 8.5496E−05 | CD8+ T-cell | 21.8317119 | 1.02E−08 | 6.17E−08 |
| Macrophages | 38.1519547 | 1.13E−11 | 4.56E−10 | CD4+ T-cell | 21.5886147 | 7.01E−14 | 6.80E−12 |
| fibroblast | 34.64529 | 4.6126E−05 | 0.00023351 | CD8+ T-cell | 20.6215407 | 9.85E−07 | 3.61E−06 |
| Macrophages | 32.9364965 | 6.48E−08 | 7.50E−07 | Gamma Delta | 20.3901357 | 6.79E−04 | 0.0016877 |
| | | | | T-Cell | | | |
| Macrophages | 32.8779969 | 1.54E−05 | 9.22E−05 | CD4+ T-cell | 20.1582351 | 4.92E−09 | 3.41E−08 |
| Macrophages | 31.9112677 | 9.99E−09 | 1.62E−07 | CD8+ T-cell | 19.5000548 | 8.43E−10 | 9.09E−09 |
| Macrophages | 31.7857177 | 6.25E−07 | 5.06E−06 | CD4+ T-cell | 19.2816021 | 1.35E−09 | 1.25E−08 |

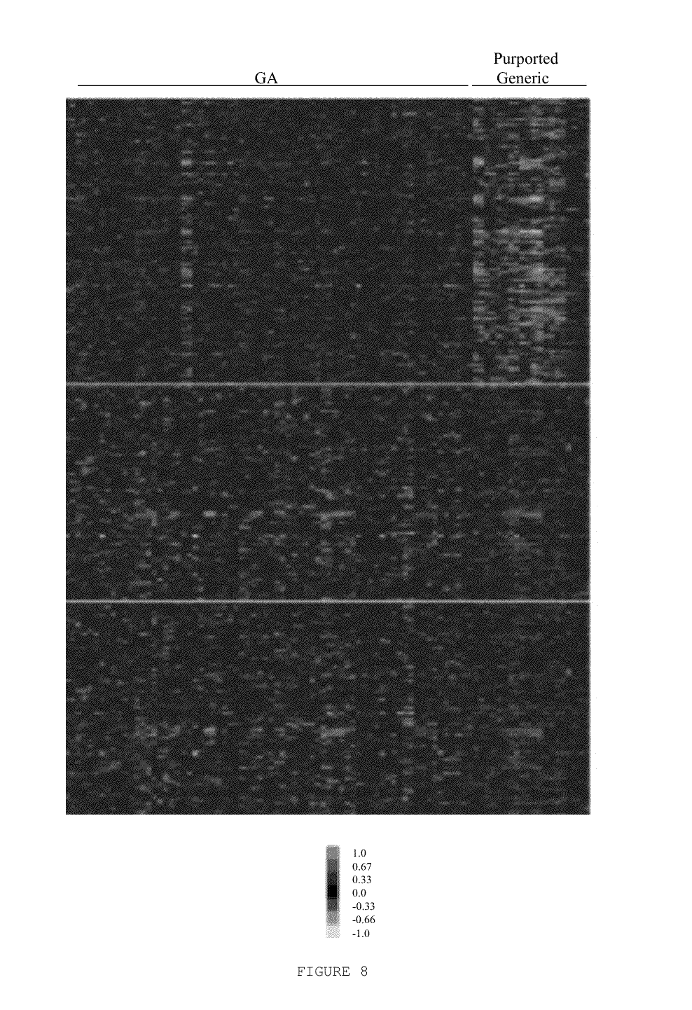

To illustrate the cell-type specificity among the genes differentially expressed between GA and PG, we created a heat map of the differentially expressed genes showing the relative expression of Treg-specific genes, macrophage-specific genes, and monocyte-specific genes in samples activated by GA compared to samples activated by PG (FIG. 8 and Table 12). Consistent with our findings, PG appears to upregulate macrophage and monocyte-related genes while GA appears to upregulate T cell related genes including Tregs.

TABLE 12
List of genes depicted in the heat map in FIG. 8.

| Row | Gene | Probe | FoxP3+T Cells |
|---|---|---|---|
| 1 | RASGRP1 | ILMN_1214318 | FoxP3+T Cells |
| 2 | IL6RA | ILMN_1216057 | FoxP3+T Cells |
| 3 | CAMK2D | ILMN_1216561 | FoxP3+T Cells |
| 4 | PRKCH | ILMN_1216897 | FoxP3+T Cells |
| 5 | ITK | ILMN_1217899 | FoxP3+T Cells |
| 6 | IQGAP2 | ILMN_1218051 | FoxP3+T Cells |
| 7 | PTPN22 | ILMN_1220975 | FoxP3+T Cells |
| 8 | ZAP70 | ILMN_1223542 | FoxP3+T Cells |
| 9 | ST8SIA4 | ILMN_1223948 | FoxP3+T Cells |
| 10 | SKAP1 | ILMN_1224032 | FoxP3+T Cells |
| 11 | SMPDL3A | ILMN_1224116 | FoxP3+T Cells |
| 12 | FAM102A | ILMN_1225348 | FoxP3+T Cells |
| 13 | ITGB7 | ILMN_1227434 | FoxP3+T Cells |
| 14 | RCAN3 | ILMN_1227610 | FoxP3+T Cells |
| 15 | TRAT1 | ILMN_1229005 | FoxP3+T Cells |
| 16 | FAAH | ILMN_1229163 | FoxP3+T Cells |
| 17 | PDK1 | ILMN_1229318 | FoxP3+T Cells |
| 18 | DD82 | ILMN_1229502 | FoxP3+T Cells |
| 19 | ECM1 | ILMN_1229746 | FoxP3+T Cells |
| 20 | CEP97 | ILMN_1230717 | FoxP3+T Cells |
| 21 | LEF1 | ILMN_1231087 | FoxP3+T Cells |
| 22 | ZNRF1 | ILMN_1232073 | FoxP3+T Cells |
| 23 | SAMHD1 | ILMN_1232707 | FoxP3+T Cells |
| 24 | IRF9 | ILMN_1233461 | FoxP3+T Cells |
| 25 | DTNB | ILMN_1233894 | FoxP3 + T Cells |
| 26 | PPP3CC | ILMN_1233999 | FoxP3 + T Cells |
| 27 | P4HA1 | ILMN_1234565 | FoxP3 + T Cells |
| 28 | FXYD5 | ILMN_1235493 | FoxP3 + T Cells |
| 29 | PROS1 | ILMN_1235499 | FoxP3 + T Cells |
| 30 | FYB | ILMN_1236105 | FoxP3 + T Cells |
| 31 | CD3E | ILMN_1238847 | FoxP3 + T Cells |
| 32 | NOTCH1 | ILMN_1241915 | FoxP3 + T Cells |
| 33 | CST7 | ILMN_1244891 | FoxP3 + T Cells |
| 34 | IL18R1 | ILMN_1244895 | FoxP3 + T Cells |
| 35 | CD84 | ILMN_1245754 | FoxP3 + T Cells |
| 36 | SSBP2 | ILMN_1246193 | FoxP3 + T Cells |
| 37 | TBXA2R | ILMN_1248837 | FoxP3 + T Cells |
| 38 | SLAMF1 | ILMN_1249863 | FoxP3 + T Cells |
| 39 | RAPGEF6 | ILMN_1254088 | FoxP3 + T Cells |
| 40 | MS4A6B | ILMN_1254692 | FoxP3 + T Cells |
| 41 | STK17B | ILMN_1255834 | FoxP3 + T Cells |
| 42 | MCOLN3 | ILMN_1256430 | FoxP3 + T Cells |
| 43 | SIT1 | ILMN_1258509 | FoxP3 + T Cells |
| 44 | SP6 | ILMN_1258571 | FoxP3 + T Cells |
| 45 | TMEM66 | ILMN_1258965 | FoxP3 + T Cells |
| 46 | IGFBP4 | ILMN_1258988 | FoxP3 + T Cells |
| 47 | TNFRSF18 | ILMN_2419490 | FoxP3 + T Cells |
| 48 | TRIB2 | ILMN_2432550 | FoxP3 + T Cells |
| 49 | TNIK | ILMN_2470251 | FoxP3 + T Cells |
| 50 | SLA2 | ILMN_2474239 | FoxP3 + T Cells |
| 51 | ZFP281 | ILMN_2477243 | FoxP3 + T Cells |
| 52 | INADL | ILMN_2490495 | FoxP3 + T Cells |
| 53 | EMB | ILMN_2547873 | FoxP3 + T Cells |
| 54 | SATB1 | ILMN_2561103 | FoxP3 + T Cells |
| 55 | SPN | ILMN_2574982 | FoxP3 + T Cells |
| 56 | CD28 | ILMN_2589871 | FoxP3 + T Cells |
| 57 | IL27RA | ILMN_2591156 | FoxP3 + T Cells |
| 58 | RNF125 | ILMN_2594109 | FoxP3 + T Cells |
| 59 | GIMAP7 | ILMN_2595918 | FoxP3 + T Cells |
| 60 | DNAHC8 | ILMN_2602341 | FoxP3 + T Cells |
| 61 | BCL11B | ILMN_2611022 | FoxP3 + T Cells |
| 62 | ARHGEF18 | ILMN_2612125 | FoxP3 + T Cells |
| 63 | MGST2 | ILMN_2613832 | FoxP3 + T Cells |
| 64 | RAB27A | ILMN_2614966 | FoxP3 + T Cells |
| 65 | DPP4 | ILMN_2615096 | FoxP3 + T Cells |
| 66 | SOCS3 | ILMN_2618176 | FoxP3 + T Cells |
| 67 | A630033H20RIK | ILMN_2622363 | FoxP3 + T Cells |
| 68 | CMAH | ILMN_2626252 | FoxP3 + T Cells |
| 69 | PELI1 | ILMN_2627441 | FoxP3 + T Cells |
| 70 | TMEM71 | ILMN_2631610 | FoxP3 + T Cells |
| 71 | THY1 | ILMN_2644350 | FoxP3 + T Cells |
| 72 | BZW2 | ILMN_2644587 | FoxP3 + T Cells |
| 73 | 4632428N05RIK | ILMN_2651297 | FoxP3 + T Cells |
| 74 | SESN1 | ILMN_2654074 | FoxP3 + T Cells |
| 75 | ITGAE | ILMN_2656090 | FoxP3 + T Cells |
| 76 | DGKA | ILMN_2658961 | FoxP3 + T Cells |
| 77 | LRBA | ILMN_2659960 | FoxP3 + T Cells |
| 78 | LAT | ILMN_2660551 | FoxP3 + T Cells |
| 79 | SCML4 | ILMN_2661185 | FoxP3 + T Cells |
| 80 | SLFN1 | ILMN_2663930 | FoxP3 + T Cells |
| 81 | PRKCQ | ILMN_2667829 | FoxP3 + T Cells |
| 82 | KIF1B | ILMN_2670778 | FoxP3 + T Cells |
| 83 | KLK8 | ILMN_2675232 | FoxP3 + T Cells |
| 84 | ZC3H12D | ILMN_2680398 | FoxP3 + T Cells |
| 85 | IL7R | ILMN_2680827 | FoxP3 + T Cells |
| 86 | BCL2 | ILMN_2682162 | FoxP3 + T Cells |
| 87 | ARID5B | ILMN_2684638 | FoxP3 + T Cells |
| 88 | PHF11 | ILMN_2696491 | FoxP3 + T Cells |
| 89 | CD3D | ILMN_2697415 | FoxP3 + T Cells |
| 90 | RGS10 | ILMN_2699531 | FoxP3 + T Cells |
| 91 | ADK | ILMN_2702471 | FoxP3 + T Cells |
| 92 | CRLF3 | ILMN_2706803 | FoxP3 + T Cells |
| 93 | GPR83 | ILMN_2707941 | FoxP3 + T Cells |
| 94 | TIMP2 | ILMN_2712867 | FoxP3 + T Cells |
| 95 | LASS4 | ILMN_2718499 | FoxP3 + T Cells |
| 96 | CTSW | ILMN_2721399 | FoxP3 + T Cells |
| 97 | CD3G | ILMN_2722784 | FoxP3 + T Cells |
| 98 | FAM134B | ILMN_2745614 | FoxP3 + T Cells |
| 99 | CD2 | ILMN_2753697 | FoxP3 + T Cells |
| 100 | ARHGAP15 | ILMN_2759756 | FoxP3 + T Cells |
| 101 | PIK3IP1 | ILMN_2769772 | FoxP3 + T Cells |
| 102 | CYB5 | ILMN_2775098 | FoxP3 + T Cells |
| 103 | LTA | ILMN_2780247 | FoxP3 + T Cells |
| 104 | NT5E | ILMN_2813830 | FoxP3 + T Cells |
| 105 | SELPLG | ILMN_2820893 | FoxP3 + T Cells |
| 106 | 1600014C10RIK | ILMN_2822842 | FoxP3 + T Cells |
| 107 | CD247 | ILMN_2828172 | FoxP3 + T Cells |
| 108 | KLHL6 | ILMN_2840514 | FoxP3 + T Cells |
| 109 | ACTN1 | ILMN_2844996 | FoxP3 + T Cells |
| 110 | INPP4B | ILMN_2854354 | FoxP3 + T Cells |
| 111 | ANXA6 | ILMN_2878060 | FoxP3 + T Cells |
| 112 | ZBP1 | ILMN_2879614 | FoxP3 + T Cells |
| 113 | IL2RG | ILMN_2886646 | FoxP3 + T Cells |
| 114 | ADD3 | ILMN_2901283 | FoxP3 + T Cells |
| 115 | FOXP3 | ILMN_2917180 | FoxP3 + T Cells |
| 116 | GBP3 | ILMN_2918002 | FoxP3 + T Cells |
| 117 | ARHGEF1 | ILMN_2920753 | FoxP3 + T Cells |
| 118 | ARL5C | ILMN_2943057 | FoxP3 + T Cells |
| 119 | DDIT4 | ILMN_2993109 | FoxP3 + T Cells |
| 120 | OBFC2A | ILMN_2996904 | FoxP3 + T Cells |
| 121 | NDRG2 | ILMN_3001650 | FoxP3 + T Cells |
| 122 | ABHD8 | ILMN_3002505 | FoxP3 + T Cells |
| 123 | SELL | ILMN_3009860 | FoxP3 + T Cells |
| 124 | CD27 | ILMN_3052501 | FoxP3 + T Cells |
| 125 | GLIPR2 | ILMN_3053593 | FoxP3 + T Cells |
| 126 | TMSB10 | ILMN_3067068 | FoxP3 + T Cells |
| 127 | GIMAP8 | ILMN_3088269 | FoxP3 + T Cells |
| 128 | GRAP2 | ILMN_3113787 | FoxP3 + T Cells |
| 129 | CD6 | ILMN_3117602 | FoxP3 + T Cells |
| 130 | A430078G23RIK | ILMN_3163163 | FoxP3 + T Cells |
| 131 | Blank | | |
| 132 | AMPD3 | ILMN_1212698 | Macrophages |
| 133 | YDJC | ILMN_1213274 | Macrophages |
| 134 | GRWD1 | ILMN_1215755 | Macrophages |
| 135 | CAMK1 | ILMN_1216632 | Macrophages |
| 136 | IFITM6 | ILMN_1218181 | Macrophages |
| 137 | PRMT7 | ILMN_1220228 | Macrophages |
| 138 | CD177 | ILMN_1225233 | Macrophages |
| 139 | TIMM10 | ILMN_1227235 | Macrophages |
| 140 | PYCRL | ILMN_1228613 | Macrophages |
| 141 | SLC43A1 | ILMN_1229667 | Macrophages |
| 142 | GSTZ1 | ILMN_1229964 | Macrophages |
| 143 | CLEC4A1 | ILMN_1230708 | Macrophages |
| 144 | TACSTD2 | ILMN_1231513 | Macrophages |
| 145 | RCL1 | ILMN_1237527 | Macrophages |
| 146 | RAI12 | ILMN_1238130 | Macrophages |
| 147 | CREG1 | ILMN_1239638 | Macrophages |
| 148 | CSF3R | ILMN_1241302 | Macrophages |
| 149 | CNPY2 | ILMN_1242881 | Macrophages |
| 150 | PLRG1 | ILMN_1244226 | Macrophages |
| 151 | KDELC1 | ILMN_1249999 | Macrophages |
| 152 | MS4A3 | ILMN_1250529 | Macrophages |
| 153 | FBXW8 | ILMN_1251236 | Macrophages |
| 154 | 1110008F13RIK | ILMN_1252407 | Macrophages |
| 155 | PRKAR2B | ILMN_1253819 | Macrophages |
| 156 | GCLM | ILMN_1256354 | Macrophages |
| 157 | HSD3B7 | ILMN_1260456 | Macrophages |
| 158 | TNFRSF21 | ILMN_2464573 | Macrophages |
| 159 | DAD1 | ILMN_2471534 | Macrophages |
| 160 | TNFAIP2 | ILMN_2474858 | Macrophages |
| 161 | PILRA | ILMN_2479717 | Macrophages |
| 162 | MRP527 | ILMN_2588199 | Macrophages |
| 163 | MT1 | ILMN_2596522 | Macrophages |
| 164 | REPIN1 | ILMN_2605767 | Macrophages |
| 165 | PYGL | ILMN_2615015 | Macrophages |
| 166 | GART | ILMN_2622163 | Macrophages |
| 167 | ACSL1 | ILMN_2622671 | Macrophages |
| 168 | POMP | ILMN_2625352 | Macrophages |
| 169 | FZR1 | ILMN_2629971 | Macrophages |
| 170 | WDR55 | ILMN_2640453 | Macrophages |
| 171 | NUP133 | ILMN_2654013 | Macrophages |
| 172 | LIG1 | ILMN_2657356 | Macrophages |
| 173 | NDUFA9 | ILMN_2657694 | Macrophages |
| 174 | GSTT3 | ILMN_2665715 | Macrophages |
| 175 | SLC6A9 | ILMN_2667384 | Macrophages |
| 176 | JOSD2 | ILMN_2667614 | Macrophages |
| 177 | SLC39A11 | ILMN_2668706 | Macrophages |
| 178 | TBRG4 | ILMN_2670230 | Macrophages |
| 179 | THOP1 | ILMN_2674324 | Macrophages |
| 180 | SLC11A1 | ILMN_2674884 | Macrophages |
| 181 | MGAT4B | ILMN_2684268 | Macrophages |
| 182 | PCYOX1L | ILMN_2684575 | Macrophages |
| 183 | SORT1 | ILMN_2685772 | Macrophages |
| 184 | BCS1L | ILMN_2691345 | Macrophages |
| 185 | DYRK3 | ILMN_2699522 | Macrophages |
| 186 | PLSCR1 | ILMN_2701271 | Macrophages |
| 187 | SYNGR2 | ILMN_2706231 | Macrophages |
| 188 | SCAMP1 | ILMN_2706853 | Macrophages |
| 189 | S100A8 | ILMN_2710905 | Macrophages |
| 190 | LCN2 | ILMN_2712075 | Macrophages |
| 191 | CHI3L3 | ILMN_2712986 | Macrophages |
| 192 | ABHD4 | ILMN_2713464 | Macrophages |
| 193 | MUC13 | ILMN_2717678 | Macrophages |
| 194 | MPO | ILMN_2719256 | Macrophages |
| 195 | POLR1B | ILMN_2723483 | Macrophages |
| 196 | INTS7 | ILMN_2726174 | Macrophages |
| 197 | RRP12 | ILMN_2728118 | Macrophages |
| 198 | SIRPA | ILMN_2744683 | Macrophages |
| 199 | FPGS | ILMN_2747070 | Macrophages |
| 200 | 2610027L16RIK | ILMN_2750842 | Macrophages |
| 201 | PRTN3 | ILMN_2758029 | Macrophages |
| 202 | GFI1B | ILMN_2772930 | Macrophages |
| 203 | TMEM176A | ILMN_2795412 | Macrophages |
| 204 | S100A9 | ILMN_2803674 | Macrophages |
| 205 | 0610007P14RIK | ILMN_2808939 | Macrophages |
| 206 | MED24 | ILMN_2818189 | Macrophages |
| 207 | TGFBI | ILMN_2834379 | Macrophages |
| 208 | CD151 | ILMN_2836710 | Macrophages |
| 209 | SLC25A39 | ILMN_2855261 | Macrophages |
| 210 | PIGQ | ILMN_2877507 | Macrophages |
| 211 | TMBIM1 | ILMN_2908056 | Macrophages |
| 212 | TREM3 | ILMN_2915303 | Macrophages |
| 213 | CTPS | ILMN_2932964 | Macrophages |
| 214 | ANXA4 | ILMN_2935012 | Macrophages |
| 215 | FBXL6 | ILMN_2935032 | Macrophages |
| 216 | TRAPPC1 | ILMN_2943661 | Macrophages |
| 217 | RASSF4 | ILMN_2956095 | Macrophages |
| 218 | TSPAN31 | ILMN_2964076 | Macrophages |
| 219 | PARP1 | ILMN_2971744 | Macrophages |
| 220 | CMTM7 | ILMN_2985447 | Macrophages |
| 221 | ASNS | ILMN_3006123 | Macrophages |
| 222 | CD63 | ILMN_3052430 | Macrophages |
| 223 | UNG | ILMN_3056503 | Macrophages |
| 224 | NAIP5 | ILMN_3059557 | Macrophages |
| 225 | ATIC | ILMN_3070951 | Macrophages |
| 226 | RBPMS | ILMN_3099398 | Macrophages |
| 227 | FTSJ1 | ILMN_3135668 | Macrophages |
| 228 | ANXA3 | ILMN_3135781 | Macrophages |
| 229 | WDR6 | ILMN_3162081 | Macrophages |
| 230 | Blank | | |
| 231 | AMPD3 | ILMN_1212698 | Monocytes |
| 232 | PLXND1 | ILMN_1214608 | Monocytes |
| 233 | CLN3 | ILMN_1214952 | Monocytes |
| 234 | CAMK1 | ILMN_1216632 | Monocytes |
| 235 | AIFM2 | ILMN_1216842 | Monocytes |
| 236 | IFITM6 | ILMN_1218181 | Monocytes |
| 237 | TM9SF4 | ILMN_1220640 | Monocytes |
| 238 | LMAN2L | ILMN_1220769 | Monocytes |
| 239 | PLEKHG3 | ILMN_1221920 | Monocytes |
| 240 | ROGDI | ILMN_1222821 | Monocytes |
| 241 | CD177 | ILMN_1225233 | Monocytes |
| 242 | SCAMP2 | ILMN_1226985 | Monocytes |
| 243 | ZEB2 | ILMN_1227793 | Monocytes |
| 244 | PIAS3 | ILMN_1227889 | Monocytes |
| 245 | PYCRL | ILMN_1228613 | Monocytes |
| 246 | CLEC4A1 | ILMN_1230708 | Monocytes |
| 247 | CREG1 | ILMN_1239638 | Monocytes |
| 248 | SLC7A7 | ILMN_1240318 | Monocytes |
| 249 | CSF3R | ILMN_1241302 | Monocytes |
| 250 | OLFM1 | ILMN_1241371 | Monocytes |
| 251 | SGPL1 | ILMN_1245609 | Monocytes |
| 252 | CASP1 | ILMN_1247592 | Monocytes |
| 253 | NQO2 | ILMN_1247930 | Monocytes |
| 254 | DUSP6 | ILMN_1248537 | Monocytes |
| 255 | KDELC1 | ILMN_1249999 | Monocytes |
| 256 | MS4A3 | ILMN_1250529 | Monocytes |
| 257 | FBXW8 | ILMN_1251236 | Monocytes |
| 258 | 1110008F13RIK | ILMN_1252407 | Monocytes |
| 259 | HSD3B7 | ILMN_1260456 | Monocytes |
| 260 | TYK2 | ILMN_2455701 | Monocytes |
| 261 | TNFRSF21 | ILMN_2464573 | Monocytes |
| 262 | DAD1 | ILMN_2471534 | Monocytes |
| 263 | TNFAIP2 | ILMN_2474858 | Monocytes |
| 264 | PILRA | ILMN_2479717 | Monocytes |
| 265 | VNN3 | ILMN_2491202 | Monocytes |
| 266 | GM962 | ILMN_2531520 | Monocytes |
| 267 | TMEM51 | ILMN_2589741 | Monocytes |
| 268 | UBE2F | ILMN_2590350 | Monocytes |
| 269 | PYGL | ILMN_2615015 | Monocytes |
| 270 | GART | ILMN_2622163 | Monocytes |
| 271 | ACSL1 | ILMN_2622671 | Monocytes |
| 272 | POMP | ILMN_2625352 | Monocytes |
| 273 | FZR1 | ILMN_2629971 | Monocytes |
| 274 | NUP133 | ILMN_2654013 | Monocytes |
| 275 | LIG1 | ILMN_2657356 | Monocytes |
| 276 | STXBP2 | ILMN_2657728 | Monocytes |
| 277 | SLC39A11 | ILMN_2668706 | Monocytes |
| 278 | SLC11A1 | ILMN_2674884 | Monocytes |
| 279 | ATP6V1B2 | ILMN_2680440 | Monocytes |
| 280 | MGAT4B | ILMN_2684268 | Monocytes |
| 281 | PCYOX1L | ILMN_2684575 | Monocytes |
| 282 | SORT1 | ILMN_2685772 | Monocytes |
| 283 | FAM129B | ILMN_2686975 | Monocytes |
| 284 | MTMR14 | ILMN_2689062 | Monocytes |
| 285 | CD44 | ILMN_2697830 | Monocytes |
| 286 | SYNGR2 | ILMN_2706231 | Monocytes |
| 287 | NLRXJ | ILMN_2708222 | Monocytes |
| 288 | S100A8 | ILMN_2710905 | Monocytes |
| 289 | LCN2 | ILMN_2712075 | Monocytes |
| 290 | 1810033B17RIK | ILMN_2712151 | Monocytes |
| 291 | CHI3L3 | ILMN_2712986 | Monocytes |
| 292 | ABHD4 | ILMN_2713464 | Monocytes |
| 293 | MPO | ILMN_2719256 | Monocytes |
| 294 | MAPK14 | ILMN_2721809 | Monocytes |
| 295 | TLR2 | ILMN_2733733 | Monocytes |
| 296 | WWP2 | ILMN_2744245 | Monocytes |
| 297 | SIRPA | ILMN_2744683 | Monocytes |
| 298 | PLEKHO2 | ILMN_2749448 | Monocytes |
| 299 | 2610027L16RIK | ILMN_2750842 | Monocytes |
| 300 | BID | ILMN_2757287 | Monocytes |
| 301 | PRTN3 | ILMN_2758029 | Monocytes |
| 302 | LRRC8D | ILMN_2766455 | Monocytes |
| 303 | TMEM38B | ILMN_2795791 | Monocytes |
| 304 | S100A9 | ILMN_2803674 | Monocytes |
| 305 | HMGCL | ILMN_2822131 | Monocytes |
| 306 | TGFBI | ILMN_2834379 | Monocytes |
| 307 | CD151 | ILMN_2836710 | Monocytes |
| 308 | SLC25A39 | ILMN_2855261 | Monocytes |
| 309 | COPZ1 | ILMN_2862093 | Monocytes |
| 310 | CSF2RA | ILMN_2866276 | Monocytes |
| 311 | CDC42BPB | ILMN_2867696 | Monocytes |
| 312 | MOCOS | ILMN_2906489 | Monocytes |
| 313 | TMBIM1 | ILMN_2908056 | Monocytes |
| 314 | TREM3 | ILMN_2915303 | Monocytes |
| 315 | ANXA4 | ILMN_2935012 | Monocytes |
| 316 | FBXL6 | ILMN_2935032 | Monocytes |
| 317 | IRAK2 | ILMN_2937735 | Monocytes |
| 318 | TRAPPC1 | ILMN_2943661 | Monocytes |
| 319 | RASSF4 | ILMN_2956095 | Monocytes |
| 320 | TSPAN31 | ILMN_2964076 | Monocytes |
| 321 | CMTM7 | ILMN_2985447 | Monocytes |
| 322 | SLC15A3 | ILMN_2987709 | Monocytes |
| 323 | NAIP5 | ILMN_3059557 | Monocytes |
| 324 | RBPMS | ILMN_3099398 | Monocytes |
| 325 | PTPN6 | ILMN_3113420 | Monocytes |
| 326 | ANXA3 | ILMN_3135781 | Monocytes |
| 327 | TBC1D2 | ILMN_3140913 | Monocytes |

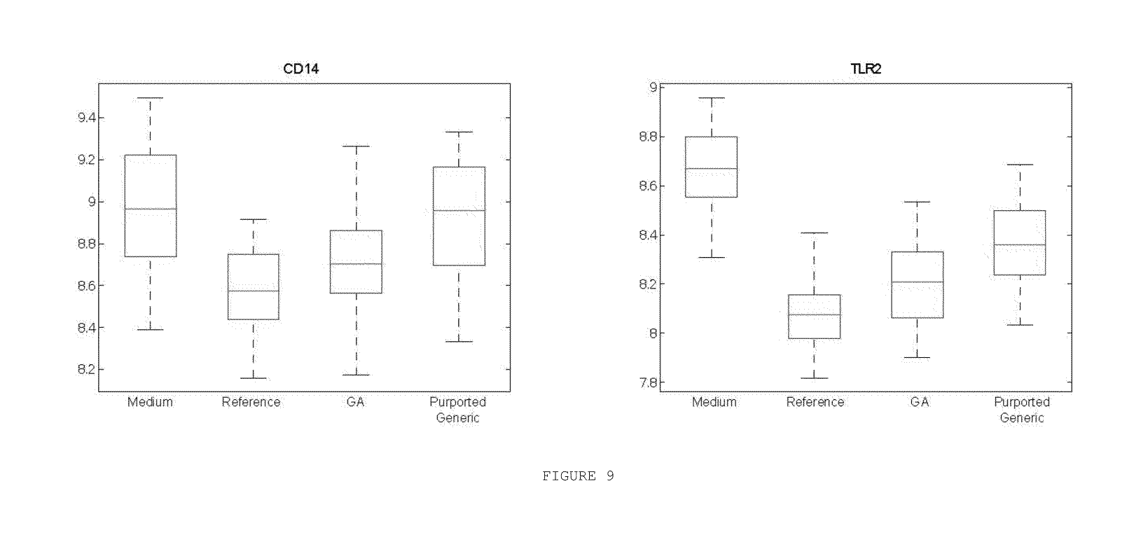

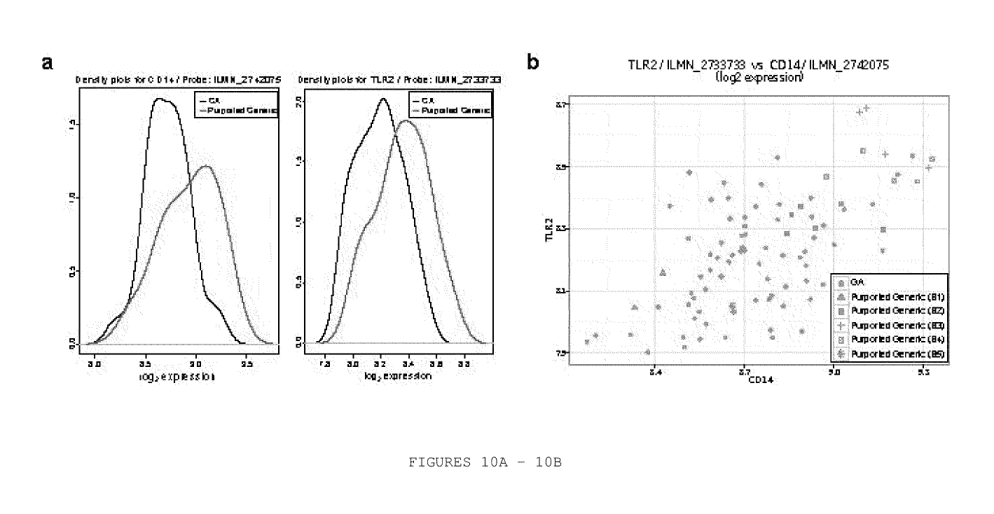

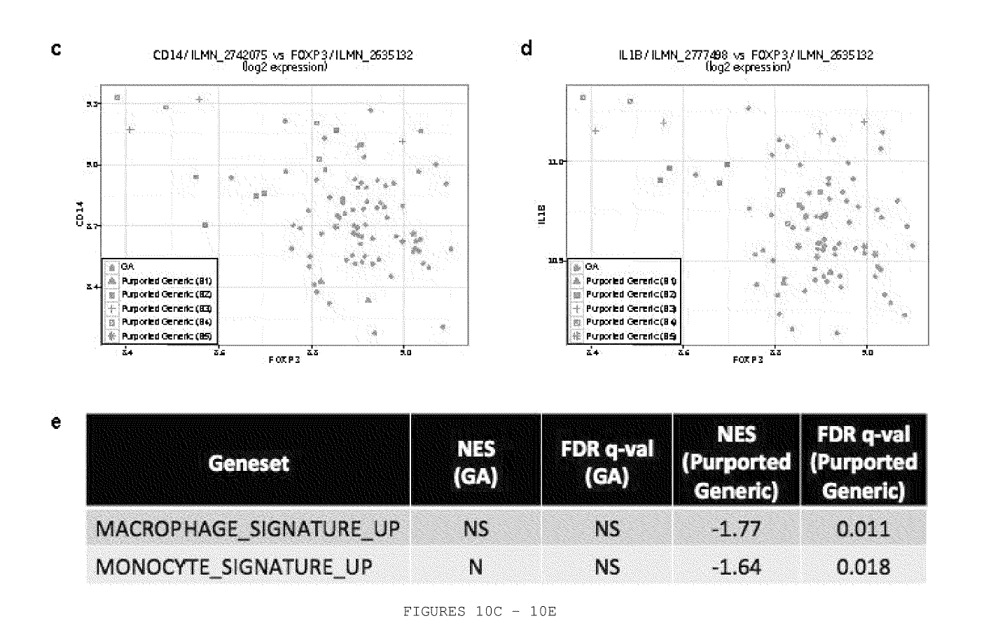

To further investigate discrepant cell type activation between the GA and PG, we utilized the non-parametric Wilcoxon rank-sum test to determine which genes had significantly higher expression from PG than from GA, and performed an enrichment using MSigDB (Methods). The TLR signaling pathway was significantly enriched (adjusted p<1.27×10−6, Table 9). Among the overlap genes significant by Wilcoxon and present in this pathway were CD14, a monocyte marker, and TLR2. Kernel density plots (FIG. 10A), which can be likened to a smoothed histogram, show the differences in expression between PG and GA for these two genes. The boxplots for these two genes can be found in FIG. 9.

Hypothesizing that both CD14 and TLR2 are associated with the same cell type (monocytes), we confirmed that the same PG samples had unusually high expression of both CD14 and TLR2 (FIG. 10B).

### EXAMPLE 10

Investigating the Mechanisms Underlying Observed Differences: Why does PG Upregulate Tregs Less Effectively than GA?

Because monocytes may play a role in the mechanisms by which GA induces Tregs, (49) we sought to compare the expression of FOXP3 and CD14 in individual samples. PG samples with low FOXP3 also have high CD14 (FIG. 10C). This suggested that the differential impact on monocytes may be one mechanism by which GA and PG differentially impact Tregs.

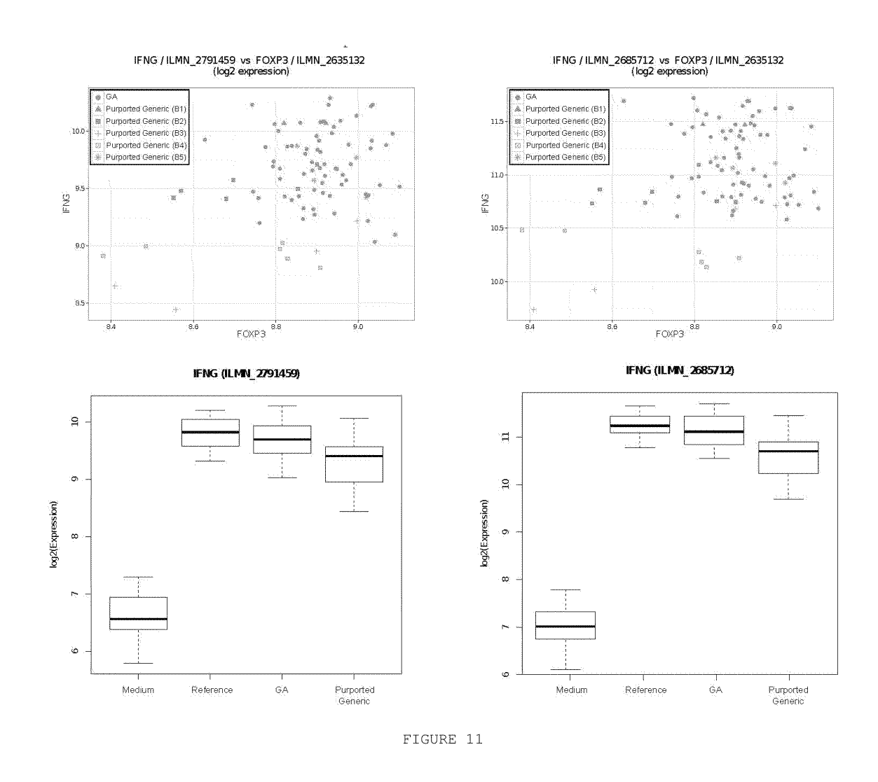

Another mechanism by which GA and PG may differentially impact Tregs involves interferon gamma, which is known to induce FOXP3 expression (50) and to be necessary for GA-induced FOXP3 expression. (51) IFNG is upregulated dramatically by GA compared to PG: probes for IFNG are the #1 and #3 ranked probes by fold change for higher expression from GA (Table 8 and FIG. 11). Indeed, those PG samples with unusually low in FOXP3 are also unusually low in IFNG (FIG. 11).

### EXAMPLE 11

Investigating the Mechanisms Underlying Observed Differences: Why does PG Upregulate Monocytes?

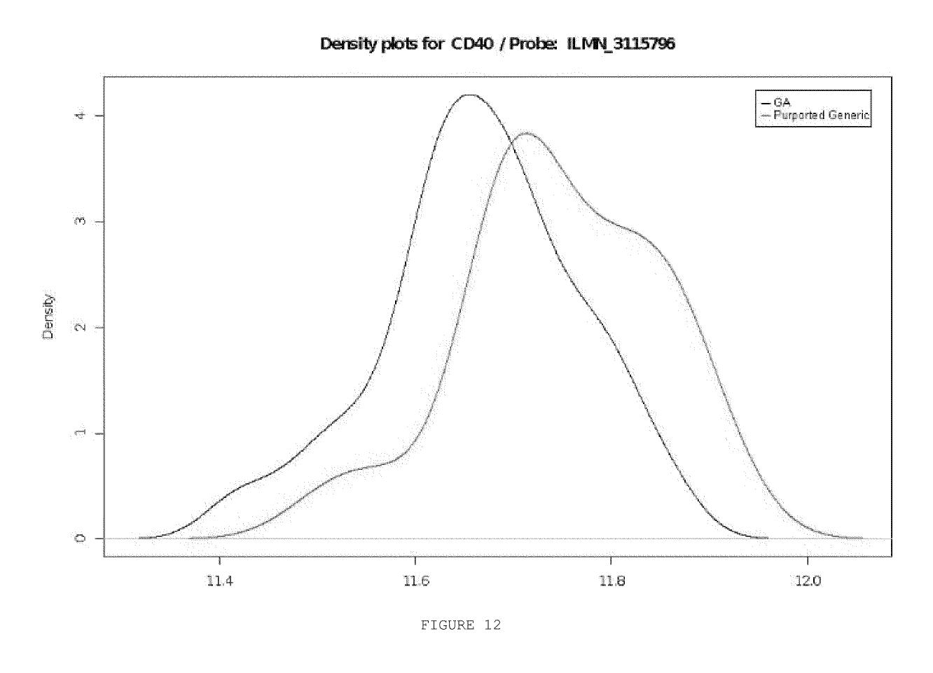

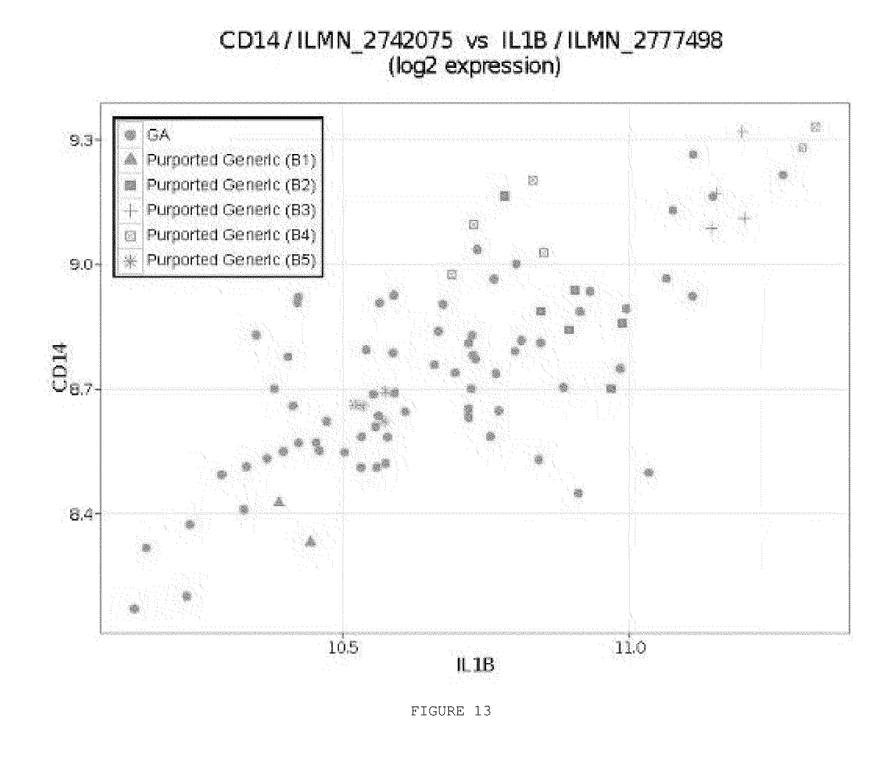

GA is known to reduce CD40 expression levels on monocytes, (49) which is consistent with our observation that CD40 is among the list of genes with significantly lower expression (Wilcoxon, Methods) following activation by GA than following activation by PG (FIG. 12). GA has a different impact on monocytes stimulated by T cell contact versus LPS: in the former case GA causes a decrease in monocyte IL1B production while in the latter case GA causes its increase. (52) This was notable because performing an MSigDB enrichment (Methods) on genes with higher expression from PG (Wilcoxon, Methods) also yielded significant enrichment in an LPS response pathway (adjusted p<4.96×10−6, Table 9). Moreover, we show that those PG samples with low levels of FOXP3 also have high levels of IL1B (FIG. 10D). GSEA analysis indicated that genes specific to monocytes and macrophages were significantly enriched among those genes with higher expression in PG than GA (FIG. 9E). IL1B also appears to be associated with monocytes, as it is highly correlated with CD14 (FIG. 13). Finally, IL1B levels are significantly higher in PG than GA both by ANOVA (adjusted p<0.043) and LIMMA with background subtraction (adjusted p<0.037)(Table 8).

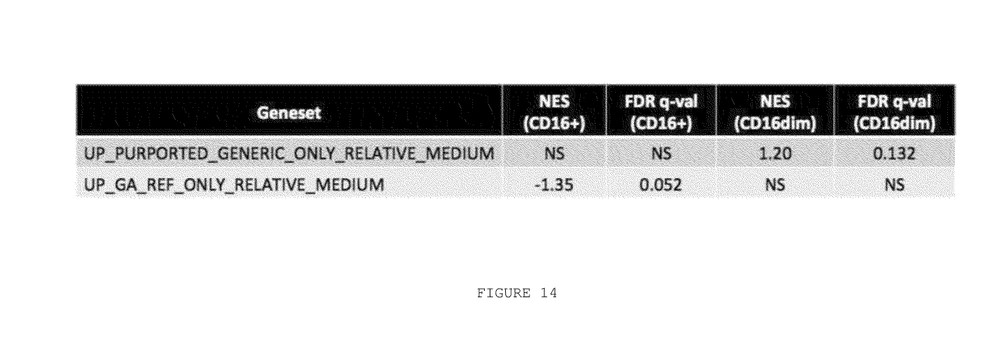

In order to determine if GA and PG were influencing different subtypes of monocytes, we performed a GSEA analysis using gene lists from literature examining human CD16+ and CD16dim monocytes. (53) We found that among the genes upregulated by PG relative to medium, there was a significant enrichment in CD16dim monocytes (FDR q=0.132, where the significance threshold is 0.25). Among the genes upregulated by GA relative to medium, there was a significant enrichment in CD16+ monocytes (FOR q=0.052, where the significance threshold is 0.25)(FIG. 14).

### Methods

### EXAMPLES 5-11

Experimental Design, Data Collection, and Pre-Processing

The experimental design, data collection, and pre-processing steps have been previously described. (15) In brief, (Balb/c×SJL) F1 mice were injected with GA reference standard in order to induce GA-reactive T cells. After 3 days the mice were sacrificed, their splenocytes were isolated, and these splenocytes were mixed with activator solutions for 24 hours. The activator solutions include multiple batches of GA, as well as glatiramer acetate reference standard, and multiple batches of PG (Glatimer, Natco Pharma, Ltd., Hyderabad, India). Then the RNA was extracted and gene expression characterized by microarray using an Illumine WG-6_V2 chip. Illumina's BeadStudio software was utilized for image processing, quantification of signal intensity per bead, and background signal correction. The microarray data have been deposited in the Gene Expression Omnibus, under accession number GSE40566.

Data Processing Steps

We quantile normalized the extracted data for all 214 samples across all 46,547 probes via the “preprocessCore” package in R (29). We then corrected for batch variation with ComBat (30) as implemented in the SVA package of R (62). Each microarray's chip designation was supplied a batch label; there were 18 batches in all. The labels for the treatments (i.e. drug product, reference standard . . . ) were added as covariates.

Variability Analysis Method

In order to identify probes with variability induced specifically by activation (as opposed to experimental noise), we sought to identify probes that were significantly more variable when activated with either GA or PG than medium. Using an F-test, we compared GA against Medium for each probe and compared PG against Medium. We then took the set of probes where either treatment comes up to be more variable than medium (union, passes in at least at least one). For those set of probes only, we compared the variability of GA to PG, utilizing an Ftest to measure significance of the differences between the probes.

Tolerance Method for Assessing Process Variability:

The goal of large scale industrial processing is to produce a large quantity of product which is of the same quality as that produced on a small scale. To assess the process consistency of GA and PG, we needed a standard of comparison. This standard of comparison was constructed by first identifying the top 1000 probes by absolute fold change of reference standard compared to the medium (Table 13).

The list includes both upregulated and downregulated probes compared to medium. Probes were filtered such that ones upregulated by reference standard needed to have an average reference standard expression of 6.00 or higher and ones downregulated by reference standard needed to have an average medium expression of 6.00 or higher. This ensured that the list of 1000 probes were both significantly affected by reference standard and were sufficiently expressed to avoid noise associated with lowly expressed probes.

TABLE 13
Genes utilized for the tolerance method illustrated in FIG. 4B.

| | | | AVG | | |
|---|---|---|---|---|---|
| | | | Reference | | |
| ID | Gene | AVG Medium | Standard | AVG GA | AVG generic |
| ILMN_2685712 | IFNG | 7.151943333 | 11.25528947 | 11.16330882 | 10.66788182 |
| ILMN_1247309 | IL3 | 5.823283333 | 9.406355263 | 9.384125 | 9.421563636 |
| ILMN_2791459 | IFNG | 6.636043333 | 9.829173684 | 9.710919118 | 9.303940909 |
| ILMN_1215862 | CXCL9 | 7.341613333 | 9.816544737 | 9.756617647 | 9.524722727 |
| ILMN_2595732 | LOC100046232 | 9.24871 | 11.50916579 | 11.54660147 | 11.76695 |
| ILMN_2931334 | IL4 | 6.171636667 | 8.367486842 | 8.425076471 | 8.496068182 |
| ILMN_2718330 | CISH | 9.18378 | 11.37349211 | 11.34956176 | 11.42528636 |
| ILMN_2445789 | TNFRSF9 | 6.794626667 | 8.943107895 | 8.962886765 | 8.867340909 |
| ILMN_1235499 | PROS1 | 6.8909 | 8.894226316 | 8.934763235 | 8.691490909 |
| ILMN_1236588 | CAR1 | 7.761993333 | 9.729336842 | 9.793017647 | 9.715413636 |
| ILMN_2588216 | IL8RA | 8.623573333 | 6.726942105 | 6.682098529 | 6.572263636 |
| ILMN_2680827 | IL7R | 10.26270333 | 8.408492105 | 8.445019118 | 8.336627273 |
| ILMN_3103746 | TNFRSF9 | 6.92238 | 8.764752632 | 8.744404412 | 8.737481818 |
| ILMN_2606162 | PDLIM4 | 7.95422 | 9.731202632 | 9.727291176 | 9.501227273 |
| ILMN_1213954 | SGK1 | 11.41363 | 9.661723684 | 9.666986765 | 9.421709091 |
| ILMN_1216042 | APOE | 9.313533333 | 7.564486842 | 7.5268 | 7.541936364 |
| ILMN_2507761 | 7530404M11RIK | 5.58119 | 7.321434211 | 7.426433824 | 7.179863636 |
| ILMN_3142803 | CXCL10 | 6.9917 | 8.700381579 | 8.707408824 | 8.518177273 |
| ILMN_1249030 | MPO | 9.78357 | 8.174247368 | 8.410351471 | 8.711063636 |
| ILMN_1214841 | IL24 | 6.20973 | 7.810955263 | 7.762489706 | 7.504672727 |
| ILMN_2744890 | GADD45G | 8.972466667 | 10.57273421 | 10.65257941 | 10.53536818 |
| ILMN_2742861 | SERPINA3F | 8.155613333 | 9.724289474 | 9.630060294 | 9.550477273 |
| ILMN_1217389 | PKIB | 9.656156667 | 11.20944737 | 11.18970735 | 11.08776818 |
| ILMN_2749412 | CSF2 | 6.444243333 | 7.991860526 | 7.939511765 | 7.916009091 |
| ILMN_2426853 | UBD | 6.649816667 | 8.167139474 | 8.137648529 | 7.952622727 |
| ILMN_2725927 | SERPINA3G | 12.45885667 | 13.95378947 | 13.90402647 | 13.98564545 |
| ILMN_3094853 | PKIB | 7.606836667 | 9.100334211 | 9.029398529 | 9.009122727 |
| ILMN_1220788 | GM590 | 6.52345 | 8.002165789 | 8.021569118 | 7.660568182 |
| ILMN_2780247 | LTA | 7.175613333 | 8.633663158 | 8.581783824 | 8.382890909 |
| ILMN_2600421 | MPO | 8.83189 | 7.385586842 | 7.594448529 | 7.965109091 |
| ILMN_2947526 | ECM1 | 8.577696667 | 10.02243947 | 10.05155441 | 9.810595455 |
| ILMN_2772264 | IGH-VJ558 | 11.98722333 | 10.55665789 | 10.57623824 | 10.44005909 |
| ILMN_1229804 | LTA | 6.55449 | 7.963042105 | 8.009302941 | 7.831009091 |
| ILMN_2506039 | A130090K04RIK | 9.575673333 | 8.169478947 | 8.136902941 | 7.924245455 |
| ILMN_2762983 | TBX21 | 7.060853333 | 8.465081579 | 8.480467647 | 8.371186364 |
| ILMN_2756046 | FFAR2 | 7.936103333 | 9.323384211 | 9.336789706 | 9.122636364 |
| ILMN_2903945 | GADD45G | 11.30813 | 12.65795526 | 12.67639853 | 12.61029091 |
| ILMN_2878071 | LYZ | 11.69561333 | 10.38226579 | 10.50047647 | 10.79755909 |
| ILMN_2460094 | WNT10A | 8.443006667 | 7.148705263 | 7.098610294 | 7.211363636 |
| ILMN_1252076 | LYZ2 | 9.709593333 | 8.418768421 | 8.645485294 | 8.893840909 |
| ILMN_1224336 | SPSB1 | 7.674476667 | 8.962918421 | 8.950477941 | 8.84225 |
| ILMN_2446559 | TNFRSF4 | 9.380393333 | 10.65664474 | 10.65343824 | 10.55523636 |
| ILMN_2717176 | RGL1 | 8.882566667 | 7.607873684 | 7.561447059 | 7.5231 |
| ILMN_2741201 | IL17F | 7.970356667 | 9.233218421 | 9.281625 | 9.150545455 |
| ILMN_3090207 | CYP4F18 | 9.138546667 | 7.882936842 | 7.951694118 | 7.8979 |
| ILMN_2546272 | GNG12 | 8.461113333 | 9.715955263 | 9.731457353 | 9.698468182 |
| ILMN_2548681 | 4930426D05RIK | 8.374753333 | 7.133186842 | 7.100458824 | 7.092509091 |
| ILMN_2664224 | EPHX1 | 11.05266667 | 9.821181579 | 9.83665 | 9.763663636 |
| ILMN_1235926 | A630072M18RIK | 7.662946667 | 8.890823684 | 8.780570588 | 8.808022727 |
| ILMN_1227900 | IL12RB1 | 6.848926667 | 8.074565789 | 8.071257353 | 8.030231818 |
| ILMN_1259075 | NME7 | 9.56043 | 8.337597368 | 8.301139706 | 8.096959091 |
| ILMN_2770066 | LOC100044439 | 10.47755333 | 9.276686842 | 9.229413235 | 9.231372727 |
| ILMN_1227434 | ITGB7 | 11.00959333 | 9.814152632 | 9.885063235 | 9.683159091 |
| ILMN_1233474 | IL2RA | 6.869136667 | 8.061205263 | 8.134330882 | 7.977345455 |
| ILMN_2906430 | ICOS | 8.19493 | 9.379094737 | 9.444848529 | 9.41605 |
| ILMN_1221819 | FCGRT | 9.812453333 | 8.634481579 | 8.525132353 | 8.270463636 |
| ILMN_2767615 | ATP1B1 | 8.774563333 | 7.597357895 | 7.592442647 | 7.479663636 |
| ILMN_2707941 | GPR83 | 8.052503333 | 9.212121053 | 9.204789706 | 8.896509091 |
| ILMN_1233987 | 1110014O20RIK | 8.7487 | 9.906276316 | 9.906113235 | 9.809395455 |
| ILMN_2758720 | SYPL | 10.40974667 | 11.56390526 | 11.58090735 | 11.63484091 |
| ILMN_2522750 | TRIO | 9.803086667 | 8.660626316 | 8.588198529 | 8.569590909 |
| ILMN_2703182 | LGALS7 | 5.84539 | 6.982352632 | 6.931479412 | 6.807209091 |
| ILMN_2729252 | 5830431A10R1K | 8.978433333 | 7.842484211 | 7.881010294 | 7.62365 |
| ILMN_1249366 | LOC100046608 | 8.23407 | 9.368084211 | 9.364801471 | 9.143586364 |
| ILMN_2670778 | KIF1B | 9.548573333 | 8.416486842 | 8.422280882 | 8.337918182 |
| ILMN_1254669 | SYPL | 9.745053333 | 10.87525263 | 10.84873088 | 10.82055 |
| ILMN_2706514 | LOC100046608 | 7.5095 | 8.634492105 | 8.612260294 | 8.444136364 |
| ILMN_2618148 | C330008K14RIK | 8.92018 | 10.04062895 | 10.14341765 | 9.885018182 |
| ILMN_2634796 | SOCS1 | 6.6267 | 7.742844737 | 7.681489706 | 7.639990909 |
| ILMN_2467190 | BAMBI-PS1 | 8.600723333 | 7.486402632 | 7.445139706 | 7.239054545 |
| ILMN_2917497 | FCGRT | 8.608183333 | 7.498360526 | 7.495925 | 7.383177273 |
| ILMN_3130403 | BC004728 | 8.17928 | 9.286213158 | 9.314702941 | 9.245672727 |
| ILMN_2758264 | CYP2S1 | 7.54357 | 6.441436842 | 6.418064706 | 6.3116 |
| ILMN_2725259 | IL2 | 6.447773333 | 7.549121053 | 7.490217647 | 7.949609091 |
| ILMN_1247198 | BC004728 | 9.205513333 | 10.29546842 | 10.36407941 | 10.27191818 |
| ILMN_2612125 | ARHGEF18 | 11.20628 | 10.12344737 | 10.09455882 | 9.868472727 |
| ILMN_1218240 | CD69 | 10.80300333 | 11.88487105 | 11.83365882 | 11.85055909 |
| ILMN_2510694 | NME7 | 8.52766 | 7.449481579 | 7.434319118 | 7.279186364 |
| ILMN_2706232 | SYNGR2 | 9.92304 | 10.98201579 | 11.08668088 | 11.19834545 |
| ILMN_2754364 | LTF | 9.291486667 | 8.233002632 | 8.257197059 | 8.373727273 |
| ILMN_2844996 | ACTN1 | 10.95206667 | 9.899202632 | 9.844863235 | 9.639622727 |
| ILMN_2596596 | RGS1 | 7.544413333 | 8.596763158 | 8.683054412 | 8.546304545 |
| ILMN_1234706 | IGHA_J00475$V00785_IG_HEAVY_{—} | 8.30976 | 7.263773684 | 7.221332353 | 7.160227273 |
| | CONSTANT_ALPHA_135 | | | | |
| ILMN_1244874 | SYNGR2 | 9.75433 | 10.79814737 | 10.86721029 | 10.9914 |
| ILMN_2724465 | CCR8 | 5.868983333 | 6.910739474 | 6.962161765 | 6.952495455 |
| ILMN_2890515 | NSG2 | 7.526546667 | 6.487660526 | 6.466486765 | 6.366909091 |
| ILMN_2690241 | PKIB | 6.76308 | 7.799152632 | 7.745076471 | 7.7524 |
| ILMN_2731237 | D8ERTD82E | 7.88429 | 8.912992105 | 8.974445588 | 9.155922727 |
| ILMN_1218037 | TMIE | 7.753076667 | 8.776018421 | 8.751725 | 8.420295455 |
| ILMN_2740407 | PDE2A | 7.706153333 | 6.685718421 | 6.704382353 | 6.679877273 |
| ILMN_2799452 | IL12RB1 | 6.439253333 | 7.457005263 | 7.404963235 | 7.448872727 |
| ILMN_2678724 | DUSP10 | 7.155803333 | 8.172573684 | 8.260263235 | 8.096786364 |
| ILMN_2669714 | CTSA | 8.099546667 | 9.113136842 | 9.205033824 | 9.180018182 |
| ILMN_2636536 | PLA2G12A | 8.84106 | 9.8543 | 9.883701471 | 9.88105 |
| ILMN_1224116 | SMPDL3A | 8.557293333 | 7.546207895 | 7.587558824 | 7.453181818 |
| ILMN_2625377 | RGS1 | 8.738546667 | 9.746213158 | 9.769272059 | 9.687659091 |
| ILMN_2635272 | IGH-VJ558 | 14.29845 | 13.29094737 | 13.22015294 | 13.18095 |
| ILMN_2534151 | IRGB10 | 8.701546667 | 9.704373684 | 9.691323529 | 9.5531 |
| ILMN_1239797 | 6030400N17RIK | 7.333296667 | 6.331097368 | 6.341064706 | 6.274213636 |
| ILMN_1249021 | BCL2 | 7.82453 | 8.822494737 | 8.747633824 | 8.542981818 |
| ILMN_2987709 | SLC15A3 | 9.175186667 | 10.16891579 | 10.19635147 | 10.39172273 |
| ILMN_2717975 | TCF7 | 9.211256667 | 8.222889474 | 8.215898529 | 8.083790909 |
| ILMN_2706231 | SYNGR2 | 10.31389 | 11.30210263 | 11.42013235 | 11.58176364 |
| ILMN_2769772 | PIK3IP1 | 8.618043333 | 7.649376316 | 7.746248529 | 7.440986364 |
| ILMN_2944824 | HP | 8.98318 | 8.016268421 | 8.174917647 | 8.30705 |
| ILMN_2947234 | CBX7 | 8.6477 | 7.682768421 | 7.728530882 | 7.684054545 |
| ILMN_1242308 | TCF7 | 9.232466667 | 8.26825 | 8.260585294 | 8.138781818 |
| ILMN_3053593 | GLIPR2 | 10.08712 | 11.04892632 | 11.06006324 | 10.93565455 |
| ILMN_2658501 | IFITM3 | 10.38816667 | 11.34819474 | 11.42799853 | 11.42086818 |
| ILMN_2668333 | PRG4 | 6.984513333 | 6.026434211 | 5.988755882 | 5.921604545 |
| ILMN_1247893 | CGEF2-PENDING | 9.050576667 | 8.096818421 | 8.110351471 | 7.883945455 |
| ILMN_1248843 | GATA3 | 7.34306 | 8.294652632 | 8.224369118 | 8.104072727 |
| ILMN_2773900 | GLIPR2 | 9.729866667 | 10.68112895 | 10.63013088 | 10.46443182 |
| ILMN_2873750 | GLDC | 5.92132 | 6.870686842 | 6.770875 | 6.741440909 |
| ILMN_2699898 | ITGAE | 8.418296667 | 7.469634211 | 7.501901471 | 7.236572727 |
| ILMN_1217629 | ITGAE | 8.464196667 | 7.527947368 | 7.552825 | 7.319981818 |
| ILMN_2897891 | RGS1 | 7.728746667 | 8.660318421 | 8.707230882 | 8.614995455 |
| ILMN_2806549 | PRM1 | 5.990036667 | 6.916326316 | 6.894220588 | 6.788813636 |
| ILMN_1229827 | ST7 | 6.727336667 | 7.649644737 | 7.737477941 | 7.809095455 |
| ILMN_1254634 | ACPL2 | 8.50204 | 7.581063158 | 7.572779412 | 7.580222727 |
| ILMN_2925094 | MPO | 7.105436667 | 6.185805263 | 6.332797059 | 6.577845455 |
| ILMN_2687652 | PRM1 | 5.935833333 | 6.854623684 | 6.849060294 | 6.777136364 |
| ILMN_3047389 | GBP2 | 7.03435 | 7.947555263 | 7.887723529 | 7.679313636 |
| ILMN_2659739 | IL7R | 7.364306667 | 6.458986842 | 6.442688235 | 6.261427273 |
| ILMN_1246543 | SIAT7C | 9.223836667 | 8.320423684 | 8.378747059 | 8.210077273 |
| ILMN_2818964 | DUSP10 | 7.31253 | 8.215365789 | 8.287358824 | 8.062272727 |
| ILMN_2600928 | OVGP1 | 7.453986667 | 6.557389474 | 6.540479412 | 6.507018182 |
| ILMN_2631752 | CTLA4 | 7.81105 | 8.706171053 | 8.619783824 | 8.483668182 |
| ILMN_2663249 | SLAMF9 | 9.11594 | 8.222615789 | 8.243369118 | 8.1212 |
| ILMN_2614889 | B3GNT8 | 9.81282 | 8.920905263 | 8.953698529 | 8.841236364 |
| ILMN_2939681 | LYZS | 7.795363333 | 6.9062 | 6.970098529 | 7.154781818 |
| ILMN_2696492 | PHF11 | 8.6652 | 9.553802632 | 9.454427941 | 9.271490909 |
| ILMN_1230045 | 4933437K13RIK | 8.913463333 | 9.798655263 | 9.824382353 | 9.735468182 |
| ILMN_2996904 | OBFC2A | 6.96731 | 7.850726316 | 7.798394118 | 7.615513636 |
| ILMN_2513451 | 1110046J11RIK | 11.05034 | 10.16700789 | 10.15531618 | 10.12564091 |
| ILMN_1217180 | IFITM1 | 10.89041667 | 11.77217632 | 11.84941765 | 11.83110455 |
| ILMN_1259174 | SCIN | 8.775283333 | 9.655794737 | 9.640454412 | 9.632631818 |
| ILMN_2481117 | 1700052O22R1K | 10.66834667 | 9.788118421 | 9.815542647 | 9.722204545 |
| ILMN_1234539 | IRGM1 | 10.58084333 | 11.46092105 | 11.438425 | 11.37995455 |
| ILMN_2611755 | SERPINB6B | 7.277453333 | 8.156736842 | 8.188377941 | 8.19805 |
| ILMN_1233293 | GBP1 | 10.84405667 | 11.71913684 | 11.66407353 | 11.49095455 |
| ILMN_2646625 | JUN | 7.34434 | 8.218031579 | 8.291385294 | 8.082372727 |
| ILMN_2761720 | LOC100041103 | 8.500646667 | 7.629236842 | 7.612494118 | 7.366713636 |
| ILMN_1237871 | AMPD1 | 7.304966667 | 6.433939474 | 6.446288235 | 6.283995455 |
| ILMN_2789650 | CSRP3 | 6.64884 | 7.517015789 | 7.601960294 | 7.724718182 |
| ILMN_2682162 | BCL2 | 7.844683333 | 8.709405263 | 8.674883824 | 8.569540909 |
| ILMN_2932359 | TPI1 | 10.31272 | 11.17369737 | 11.19473235 | 10.92615455 |
| ILMN_1214071 | IFITM1 | 10.60328 | 11.4639 | 11.54568971 | 11.61521364 |
| ILMN_1242622 | CREM | 7.411453333 | 8.271910526 | 8.247942647 | 8.217618182 |
| ILMN_1237338 | DDX25 | 7.421036667 | 6.560686842 | 6.532476471 | 6.45865 |
| ILMN_1218525 | IL18R1 | 8.817636667 | 7.958268421 | 7.945545588 | 7.754745455 |
| ILMN_2636403 | AXUD1 | 9.995656667 | 10.85401842 | 10.84707206 | 10.69045909 |
| ILMN_2578341 | B930094H08R1K | 7.454906667 | 6.597310526 | 6.497302941 | 6.428463636 |
| ILMN_2658878 | TG | 6.5678 | 7.422852632 | 7.479155882 | 7.382472727 |
| ILMN_2726471 | EN2 | 5.743626667 | 6.598660526 | 6.582991176 | 6.530154545 |
| ILMN_1214294 | SNX29 | 6.848423333 | 7.702268421 | 7.713407353 | 7.705336364 |
| ILMN_2646322 | SAMSN1 | 10.94873 | 11.79982368 | 11.86731176 | 11.88902273 |
| ILMN_2777019 | SPO11 | 7.814436667 | 6.967128947 | 6.908411765 | 6.728213636 |
| ILMN_2678726 | RASGRP2 | 7.477166667 | 6.636023684 | 6.733875 | 6.734577273 |
| ILMN_2700689 | IBRDC3 | 8.3528 | 9.193568421 | 9.290398529 | 9.354804545 |
| ILMN_1244866 | GBP5 | 8.2183 | 9.058268421 | 9.012258824 | 8.881472727 |
| ILMN_2765759 | ASB2 | 8.020873333 | 8.860181579 | 8.794510294 | 8.691077273 |
| ILMN_1239878 | PRSS34 | 7.13797 | 6.298813158 | 6.278086765 | 6.330131818 |
| ILMN_2689401 | MCOLN2 | 8.4654 | 7.62645 | 7.5921 | 7.59695 |
| ILMN_2447712 | IBRDC3 | 6.955663333 | 7.792826316 | 7.789882353 | 7.901122727 |
| ILMN_1223600 | INDO | 6.495596667 | 7.332621053 | 7.296913235 | 7.166577273 |
| ILMN_2751603 | PHXR4 | 9.941726667 | 9.109602632 | 8.973426471 | 8.817872727 |
| ILMN_1221516 | LOC100048841 | 6.434853333 | 7.2654 | 7.302933824 | 7.260995455 |
| ILMN_2419494 | TNFR5F18 | 9.906223333 | 10.73479474 | 10.79889559 | 10.57273636 |
| ILMN_1214783 | A530050E01RIK | 8.485986667 | 7.659539474 | 7.615842647 | 7.62025 |
| ILMN_1239729 | KIF1B | 8.392053333 | 7.567934211 | 7.516839706 | 7.370368182 |
| ILMN_1233501 | C530027B15R1K | 9.48782 | 8.663984211 | 8.683922059 | 8.504722727 |
| ILMN_1253182 | HS3ST1 | 8.033893333 | 8.855842105 | 8.805114706 | 8.860563636 |
| ILMN_2638491 | SLAMF7 | 7.82434 | 8.644642105 | 8.627872059 | 8.595940909 |
| ILMN_2592953 | SCG5 | 7.77508 | 6.956702632 | 6.960557353 | 6.79455 |
| ILMN_1218036 | SCO1 | 8.34345 | 9.161613158 | 9.071594118 | 9.090790909 |
| ILMN_1229840 | FCRLA | 10.64000667 | 9.823918421 | 9.907154412 | 9.789463636 |
| ILMN_1260046 | SFT2D2 | 9.51672 | 10.33144474 | 10.2783 | 10.18119545 |
| ILMN_2662097 | RAB35 | 10.63486 | 11.44911053 | 11.42574559 | 11.5381 |
| ILMN_2696491 | PHF11 | 8.4808 | 9.294965789 | 9.237983824 | 9.094609091 |
| ILMN_1249824 | IGF1R | 7.569356667 | 6.755618421 | 6.779692647 | 6.7302 |
| ILMN_2789900 | CD177 | 7.385106667 | 6.571739474 | 6.62825 | 6.835731818 |
| ILMN_2665545 | RIN3 | 9.836286667 | 9.024592105 | 9.014795588 | 8.852218182 |
| ILMN_3122961 | GBP2 | 12.90305333 | 13.71437895 | 13.71902353 | 13.60109545 |
| ILMN_2602899 | CREM | 6.903246667 | 7.7135 | 7.713844118 | 7.7371 |
| ILMN_1258965 | TMEM66 | 11.24646333 | 10.43824474 | 10.41262794 | 10.26992273 |
| ILMN_1238725 | C130086J11RIK | 6.425446667 | 7.233239474 | 7.187627941 | 7.029145455 |
| ILMN_2855315 | HIST1H1C | 9.337553333 | 8.530276316 | 8.505617647 | 8.621104545 |
| ILMN_2658961 | DGKA | 11.82494 | 11.01769737 | 11.06722353 | 10.9061 |
| ILMN_1217723 | STAU2 | 7.913883333 | 8.719473684 | 8.736936765 | 8.7426 |
| ILMN_2431237 | XBP1 | 11.49460333 | 12.30005789 | 12.26956618 | 12.30432273 |
| ILMN_2619200 | ERAF | 7.865656667 | 8.669657895 | 8.714435294 | 8.671418182 |
| ILMN_1234698 | TSPAN2 | 6.670486667 | 7.473368421 | 7.425954412 | 7.439659091 |
| ILMN_2795178 | RFTN2 | 8.005233333 | 7.209273684 | 7.217733824 | 7.237681818 |
| ILMN_1258283 | LTB | 11.83477333 | 11.042742111 | 1.03906324 | 10.91391364 |
| ILMN_2643513 | ASNS | 9.04453 | 9.835744737 | 9.741755882 | 9.861822727 |
| ILMN_1258357 | DOK3 | 12.26605 | 11.47897632 | 11.44765 | 11.44477727 |
| ILMN_2491741 | TRIO | 7.336273333 | 6.549247368 | 6.611138235 | 6.557227273 |
| ILMN_2492284 | AA536717 | 7.253643333 | 6.468510526 | 6.484120588 | 6.349872727 |
| ILMN_3128992 | CD27 | 11.71615 | 10.93310263 | 10.95738971 | 10.65876364 |
| ILMN_2742068 | CSRP3 | 6.410026667 | 7.191834211 | 7.259801471 | 7.395309091 |
| ILMN_2419490 | TNFRSF18 | 10.22464333 | 11.00578158 | 11.07498088 | 10.83858636 |
| ILMN_2650953 | SLC12A7 | 7.512276667 | 6.731831579 | 6.744029412 | 6.646413636 |
| ILMN_2749063 | DOCK10 | 7.141206667 | 7.920371053 | 7.865589706 | 7.847540909 |
| ILMN_2807084 | ACOT7 | 8.594826667 | 9.372 | 9.352392647 | 9.372054545 |
| ILMN_2473692 | 1110059G02RIK | 7.65595 | 6.880018421 | 6.850135294 | 6.816413636 |
| ILMN_1237695 | PFKP | 9.860906667 | 10.63633421 | 10.65613824 | 10.56410455 |
| ILMN_2592486 | genericLYRP1 | 8.485243333 | 7.710365789 | 7.785183824 | 7.734190909 |
| ILMN_2630993 | PPAP2B | 8.248056667 | 7.473742105 | 7.506554412 | 7.530063636 |
| ILMN_3006767 | MLKL | 7.866353333 | 8.638142105 | 8.575160294 | 8.697018182 |
| ILMN_1244343 | B230369L08RIK | 10.64261667 | 9.871126316 | 9.899548529 | 9.819563636 |
| ILMN_2888191 | CCR5 | 7.154613333 | 7.925273684 | 7.880173529 | 7.807190909 |
| ILMN_1248413 | FASL | 6.024673333 | 6.795171053 | 6.780129412 | 6.697140909 |
| ILMN_2803674 | S100A9 | 11.68038333 | 10.91212105 | 11.05074559 | 11.24515455 |
| ILMN_2691014 | KLHL14 | 7.10078 | 6.333918421 | 6.30465 | 6.381831818 |
| ILMN_2725448 | SYTL3 | 6.27369 | 7.040426316 | 7.029935294 | 6.914209091 |
| ILMN_2666272 | HEMK1 | 8.380666667 | 9.14595 | 9.173805882 | 9.25 |
| ILMN_2540103 | LOC666559 | 9.92083 | 10.68557895 | 10.7199 | 10.70793636 |
| ILMN_2488997 | 2010005O13RIK | 9.116286667 | 9.880084211 | 9.797192647 | 9.668695455 |
| ILMN_3137287 | LIF | 5.682976667 | 6.44575 | 6.403722059 | 6.416013636 |
| ILMN_2668510 | HP | 8.153523333 | 7.391392105 | 7.507407353 | 7.527177273 |
| ILMN_2646891 | CENTD1 | 10.04395333 | 10.80583684 | 10.76900441 | 10.81540909 |
| ILMN_2614161 | LSS | 8.567596667 | 9.328973684 | 9.397113235 | 9.497386364 |
| ILMN_2976440 | BLK | 10.83170333 | 10.07106842 | 10.05113971 | 10.12329545 |
| ILMN_2654074 | SESN1 | 11.24036667 | 10.47988947 | 10.44926176 | 10.32896818 |
| ILMN_2593143 | DOCK10 | 10.52829667 | 11.28851579 | 11.30300882 | 11.33595 |
| ILMN_1239397 | LOC100047934 | 9.405073333 | 10.16416842 | 10.16762353 | 10.09550909 |
| ILMN_2668509 | HP | 7.943053333 | 7.184218421 | 7.287372059 | 7.381036364 |
| ILMN_1232621 | ANK | 7.856653333 | 7.099386842 | 7.122891176 | 7.169036364 |
| ILMN_1221700 | ELA2 | 7.074613333 | 6.318086842 | 6.367277941 | 6.59045 |
| ILMN_2734391 | RAMP1 | 7.914883333 | 7.158755263 | 7.212301471 | 6.993427273 |
| ILMN_1230137 | TPI1 | 11.74445667 | 12.50032895 | 12.51968382 | 12.25161818 |
| ILMN_1234746 | AI790298 | 7.578593333 | 6.822742105 | 6.873454412 | 6.792159091 |
| ILMN_2648454 | SIGLECG | 10.3029 | 9.547089474 | 9.551536765 | 9.551795455 |
| ILMN_2716622 | MAPK11 | 9.600273333 | 8.844921053 | 8.852513235 | 8.744913636 |
| ILMN_2685023 | HMHA1 | 12.16146 | 11.40637895 | 11.42380441 | 11.32649091 |
| ILMN_1231814 | CCL5 | 9.356793333 | 8.602223684 | 8.662139706 | 8.673418182 |
| ILMN_2818246 | CPNE4 | 7.812076667 | 7.057652632 | 7.052097059 | 6.961063636 |
| ILMN_1254685 | EG240327 | 6.397796667 | 7.151681579 | 7.153916176 | 7.099763636 |
| ILMN_2940446 | DGKA | 11.23734667 | 10.48404737 | 10.49755588 | 10.33128182 |
| ILMN_2862379 | RHOD | 7.05575 | 6.304131579 | 6.303676471 | 6.307731818 |
| ILMN_2733778 | IL4I1 | 12.624 | 13.37522368 | 13.390125 | 13.45152273 |
| ILMN_2545445 | HIPK2 | 6.55741 | 7.308373684 | 7.332480882 | 7.280054545 |
| ILMN_2700168 | CCND2 | 10.03954667 | 10.79047632 | 10.82301765 | 10.75205455 |
| ILMN_2618918 | SLC2A6 | 9.982256667 | 10.73254211 | 10.75579118 | 10.77306364 |
| ILMN_2881296 | TMEM66 | 10.33295333 | 9.5849 | 9.557625 | 9.396040909 |
| IIMN_2700166 | CCND2 | 10.59667667 | 11.34464211 | 11.37600588 | 11.32170909 |
| ILMN_2696609 | MSCP | 8.963526667 | 8.215607895 | 8.264191176 | 8.327386364 |
| ILMN_1234973 | 4630004K05RIK | 6.210026667 | 6.954189474 | 6.972236765 | 6.985181818 |
| ILMN_2492264 | WISP1 | 5.968076667 | 6.711328947 | 6.711036765 | 6.729045455 |
| ILMN_2606180 | ACOT7 | 8.450883333 | 9.193897368 | 9.205269118 | 9.229513636 |
| ILMN_1233589 | CD27 | 8.96667 | 8.224807895 | 8.296473529 | 8.010390909 |
| ILMN_2459899 | ADAMTSL4 | 7.314856667 | 8.055031579 | 8.038655882 | 7.792595455 |
| ILMN_2617468 | CHAC1 | 7.318223333 | 8.0581 | 8.051151471 | 8.204913636 |
| ILMN_1225730 | FDPS | 11.93995 | 12.67975 | 12.64576029 | 12.72600455 |
| ILMN_1239632 | NG23 | 9.102366667 | 8.364705263 | 8.462726471 | 8.421986364 |
| ILMN_2833243 | C330023M02RIK | 9.257843333 | 9.993915789 | 9.987714706 | 9.956754545 |
| ILMN_3131478 | BCAT1 | 8.48867 | 9.224363158 | 9.234775 | 9.319163636 |
| ILMN_2734212 | CD1D1 | 8.991273333 | 8.255613158 | 8.262241176 | 8.221804545 |
| ILMN_2615096 | DPP4 | 10.40495 | 9.669584211 | 9.690317647 | 9.540363636 |
| ILMN_1258864 | BC106179 | 8.446476667 | 7.711865789 | 7.749288235 | 7.574309091 |
| ILMN_1229197 | OTTMUSG00000016644 | 8.368543333 | 7.633965789 | 7.618216176 | 7.357127273 |
| ILMN_1255743 | IL6RA | 8.160456667 | 7.426255263 | 7.478202941 | 7.268536364 |
| ILMN_2560567 | A630006E02RIK | 10.59341 | 9.863042105 | 9.936775 | 9.685986364 |
| ILMN_2619107 | LGALS1 | 9.918706667 | 10.6487 | 10.70566324 | 10.63135 |
| ILMN_2619316 | PRNP | 9.040923333 | 9.770686842 | 9.719486765 | 9.604395455 |
| ILMN_2960108 | CYP27A1 | 6.88932 | 6.160155263 | 6.166485294 | 6.144959091 |
| ILMN_3153982 | NACC2 | 7.91061 | 7.181697368 | 7.109570588 | 7.017036364 |
| ILMN_2695047 | PNPLA7 | 8.31495 | 7.586323684 | 7.637077941 | 7.563486364 |
| ILMN_2976441 | BLK | 11.54462667 | 10.81708684 | 10.82114853 | 10.89478636 |
| ILMN_3136744 | SESN1 | 10.62493 | 9.897607895 | 9.837792647 | 9.802745455 |
| ILMN_2710905 | 5100A8 | 13.23183 | 12.50617368 | 12.62048529 | 12.83921818 |
| ILMN_1256644 | SLC6A12 | 7.60547 | 6.879828947 | 6.942016176 | 7.050290909 |
| ILMN_2639036 | HSPD1 | 8.82105 | 9.546586842 | 9.439136765 | 9.368622727 |
| ILMN_2490076 | PKIB | 6.435753333 | 7.161171053 | 7.095933824 | 7.160954545 |
| ILMN_1232668 | MAD | 7.291183333 | 8.015381579 | 7.986783824 | 7.835204545 |
| ILMN_2828768 | USP28 | 8.408456667 | 7.685742105 | 7.665252941 | 7.660695455 |
| ILMN_2860649 | GBP6 | 7.584156667 | 8.306197368 | 8.263667647 | 8.019059091 |
| ILMN_2517611 | 7530408C15RIK | 7.806793333 | 7.085144737 | 7.035369118 | 6.93015 |
| ILMN_2865016 | CD83 | 11.44668667 | 12.16742368 | 12.24165294 | 12.34662273 |
| ILMN_1220101 | EBI2 | 10.51159667 | 11.23125263 | 11.28764265 | 11.27350909 |
| ILMN_2715042 | SDC3 | 8.413436667 | 9.132221053 | 9.146326471 | 9.056245455 |
| ILMN_2860645 | GBP6 | 8.79986 | 9.517697368 | 9.558605882 | 9.364745455 |
| ILMN_1248028 | EMP3 | 10.66351333 | 9.946255263 | 10.05402647 | 10.04704545 |
| ILMN_1220236 | CTSG | 8.164396667 | 7.447392105 | 7.593658824 | 7.725227273 |
| ILMN_2774537 | HIST1H1C | 8.914876667 | 8.198747368 | 8.165147059 | 8.258654545 |
| ILMN_2678127 | RNF144A | 8.33433 | 7.618615789 | 7.589805882 | 7.484554545 |
| ILMN_3162239 | PRR7 | 7.936 | 7.221010526 | 7.209419118 | 7.151090909 |
| ILMN_1255860 | KLRD1 | 9.2035 | 8.489178947 | 8.505567647 | 8.348959091 |
| ILMN_1212653 | RFTN2 | 7.837833333 | 7.125721053 | 7.108895588 | 7.111963636 |
| ILMN_2457437 | A830080H07RIK | 10.58319667 | 9.872428947 | 9.842497059 | 9.785954545 |
| ILMN_2852672 | TSPAN32 | 10.27871 | 9.570586842 | 9.543492647 | 9.500654545 |
| ILMN_1254314 | 4931429I11RIK | 7.49642 | 6.788428947 | 6.757355882 | 6.756540909 |
| ILMN_2696610 | SLC25A37 | 8.959986667 | 8.252265789 | 8.278280882 | 8.350490909 |
| ILMN_2712986 | CHI3L3 | 8.252926667 | 7.545768421 | 7.58855 | 7.786881818 |
| ILMN_1227277 | D9ERTD392E | 10.15505 | 9.448323684 | 9.413408824 | 9.290313636 |
| ILMN_3005441 | PPA1 | 11.77690333 | 12.47936316 | 12.44846176 | 12.45698636 |
| ILMN_2553041 | A130093I21RIK | 8.614756667 | 7.912957895 | 7.931957353 | 7.739868182 |
| ILMN_2457571 | MAML2 | 9.231806667 | 8.530407895 | 8.504491176 | 8.382118182 |
| ILMN_3103896 | TIMP1 | 6.028073333 | 6.728947368 | 6.733719118 | 6.625322727 |
| ILMN_2758029 | PRTN3 | 7.127993333 | 6.427576316 | 6.471620588 | 6.740686364 |
| ILMN_1221048 | PSAT1 | 9.916976667 | 10.61730789 | 10.49174706 | 10.59564545 |
| ILMN_2757569 | ENO3 | 9.369076667 | 8.668905263 | 8.710376471 | 8.733259091 |
| ILMN_2804559 | TMEM108 | 8.12613 | 7.426189474 | 7.472713235 | 7.367513636 |
| ILMN_2690460 | JAKMIP1 | 10.34174667 | 9.644271053 | 9.597117647 | 9.541822727 |
| ILMN_2883326 | PIRA3 | 8.234013333 | 7.537413158 | 7.603591176 | 7.675386364 |
| ILMN_2675922 | 2310047A01RIK | 8.327533333 | 7.632757895 | 7.605654412 | 7.557727273 |
| ILMN_1228804 | A430106G13RIK | 7.31336 | 6.618973684 | 6.595263235 | 6.54365 |
| ILMN_1239055 | KDH | 6.36512 | 7.058510526 | 7.064069118 | 6.989463636 |
| ILMN_2650739 | ARHGAP9 | 9.78214 | 9.089992105 | 9.140601471 | 9.032004545 |
| ILMN_2500370 | 9430080K19RIK | 10.87341 | 10.18259737 | 10.17046471 | 10.11225 |
| ILMN_2611532 | SLC25A19 | 8.19492 | 8.885142105 | 9.008430882 | 9.032790909 |
| ILMN_2735615 | ISG20 | 7.518246667 | 8.208171053 | 8.139072059 | 8.239459091 |
| ILMN_3156343 | WARS | 8.263773333 | 8.951560526 | 8.995901471 | 8.8753 |
| ILMN_3157081 | SYTL3 | 6.38456 | 7.070873684 | 7.083748529 | 7.023881818 |
| ILMN_2632264 | ACOT7 | 10.69470667 | 11.38071053 | 11.36450147 | 11.44196818 |
| ILMN_2631610 | TMEM71 | 7.29944 | 6.615542105 | 6.536094118 | 6.333768182 |
| ILMN_2754158 | TGTP | 7.284873333 | 7.967889474 | 7.893519118 | 7.793495455 |
| ILMN_3018445 | CYP4F18 | 7.031773333 | 6.349197368 | 6.342822059 | 6.333136364 |
| ILMN_1258364 | 5430434G16RIK | 9.318306667 | 8.636010526 | 8.610383824 | 8.485986364 |
| ILMN_2660182 | ACOT7 | 7.83013 | 8.510831579 | 8.549666176 | 8.507154545 |
| ILMN_2971142 | AMPD1 | 6.766323333 | 6.086144737 | 6.075851471 | 6.011377273 |
| ILMN_1244516 | RASGRP2 | 7.476703333 | 6.7969 | 6.799802941 | 6.836954545 |
| ILMN_2703829 | FCRLA | 12.25565 | 11.57618421 | 11.55282353 | 11.51362273 |
| ILMN_2629191 | CPM | 9.91393 | 9.234957895 | 9.169702941 | 9.147559091 |
| ILMN_2979248 | TMED8 | 8.15378 | 8.831739474 | 8.811263235 | 8.855245455 |
| ILMN_1221530 | ADCY7 | 9.032993333 | 8.355923684 | 8.271894118 | 8.166209091 |
| ILMN_1236111 | FCRLA | 12.55218 | 11.87590263 | 11.88261912 | 11.83079091 |
| ILMN_1213609 | TXNIP | 12.58893667 | 11.91322368 | 11.88612353 | 11.75423182 |
| ILMN_2816180 | LBH | 11.77342667 | 11.09807368 | 11.09418676 | 11.01050455 |
| ILMN_2606072 | AGFG1 | 8.354076667 | 9.028615789 | 9.006667647 | 8.942363636 |
| ILMN_2751111 | 4931429111RIK | 7.654283333 | 6.980063158 | 6.943754412 | 6.948795455 |
| ILMN_1234054 | TMED8 | 7.90326 | 8.575631579 | 8.518032353 | 8.587863636 |
| ILMN_2766875 | OCIAD2 | 6.029033333 | 6.699218421 | 6.716430882 | 6.700781818 |
| ILMN_2524763 | 4930597A21RIK | 8.344646667 | 7.674497368 | 7.673958824 | 7.653013636 |
| ILMN_3006123 | ASNS | 7.223503333 | 7.891473684 | 7.884744118 | 7.997090909 |
| ILMN_2628174 | ACSS1 | 10.95242667 | 10.28503158 | 10.25702794 | 10.11349091 |
| ILMN_1233402 | LOC100045981 | 10.00150667 | 9.334863158 | 9.334583824 | 9.159545455 |
| ILMN_1237074 | SLC25A19 | 11.00412333 | 11.66939737 | 11.67991765 | 11.73784545 |
| ILMN_2852034 | HIF1A | 7.287836667 | 7.951565789 | 7.906245588 | 7.818386364 |
| ILMN_1250410 | EIF2AK2 | 9.431796667 | 10.09508684 | 10.06052647 | 9.927563636 |
| ILMN_2837493 | EG634650 | 5.993646667 | 6.656815789 | 6.735947059 | 6.467818182 |
| ILMN_2754698 | CD84 | 10.28207333 | 9.619726316 | 9.660651471 | 9.608509091 |
| ILMN_1256356 | PML | 10.07259 | 10.73383421 | 10.68198088 | 10.62860455 |
| ILMN_1239472 | CEACAM2 | 7.422096667 | 6.760892105 | 6.785801471 | 6.778659091 |
| ILMN_2421220 | BRWD1 | 8.272236667 | 7.614028947 | 7.590058824 | 7.579027273 |
| ILMN_1233999 | PPP3CC | 8.017933333 | 8.674994737 | 8.723741176 | 8.655763636 |
| ILMN_1245731 | HHEX | 10.25222333 | 9.595684211 | 9.542633824 | 9.5176 |
| ILMN_2656854 | MYO6 | 7.86478 | 8.521181579 | 8.467532353 | 8.266945455 |
| ILMN_2731760 | MYO1F | 9.27305 | 8.617213158 | 8.592685294 | 8.631081818 |
| ILMN_2642462 | GTF2I | 12.18328 | 11.52754211 | 11.51643971 | 11.44292727 |
| ILMN_1218533 | A630054M16RIK | 8.19438 | 8.849878947 | 8.827914706 | 8.862331818 |
| ILMN_3115917 | HSD11B1 | 9.131606667 | 8.476536842 | 8.515530882 | 8.443059091 |
| ILMN_2945940 | RAgenericEF4 | 7.861843333 | 7.20755 | 7.202657353 | 7.017481818 |
| ILMN_2433990 | LOC100048346 | 8.55353 | 9.207039474 | 9.203336765 | 9.144786364 |
| ILMN_2752436 | 2210010N04RIK | 7.966906667 | 7.314218421 | 7.364426471 | 7.283672727 |
| ILMN_1258197 | D230004N01RIK | 7.73881 | 8.391144737 | 8.361938235 | 8.371745455 |
| ILMN_1218181 | IFITM6 | 6.918566667 | 6.268265789 | 6.280010294 | 6.432531818 |
| ILMN_2742042 | NUPR1 | 6.801553333 | 7.451723684 | 7.512701471 | 7.494413636 |
| ILMN_3112873 | TXNIP | 11.50670667 | 10.85741053 | 10.80823824 | 10.65764545 |
| ILMN_1254405 | LMNA | 8.1473 | 8.796184211 | 8.835444118 | 8.852681818 |
| ILMN_2705878 | LIMD2 | 8.693046667 | 8.044581579 | 8.037157353 | 7.890031818 |
| ILMN_2594525 | NSDHL | 7.7064 | 8.354552632 | 8.365227941 | 8.322163636 |
| ILMN_2606825 | CMAH | 8.312323333 | 7.6647 | 7.634252941 | 7.396363636 |
| ILMN_2522460 | 3010031K01RIK | 8.543816667 | 9.191336842 | 9.234385294 | 9.170268182 |
| ILMN_3132898 | YPEL3 | 13.39026333 | 12.74307368 | 12.76953824 | 12.67990455 |
| ILMN_1243621 | A130026C10RIK | 9.7118 | 9.064681579 | 9.029929412 | 8.839186364 |
| ILMN_2751037 | SPATA13 | 11.19429 | 10.54886053 | 10.48956912 | 10.47108636 |
| ILMN_1232235 | THADA | 9.334693333 | 8.690102632 | 8.651617647 | 8.673077273 |
| ILMN_1254157 | WARS | 9.735603333 | 10.37886316 | 10.35691912 | 10.27182273 |
| ILMN_2777769 | H2-DMA | 11.86790667 | 12.51002368 | 12.53100441 | 12.56078636 |
| ILMN_1228832 | NGP | 8.59455 | 7.952828947 | 8.124844118 | 8.267295455 |
| ILMN_1219896 | LOC623121 | 9.744836667 | 9.106268421 | 9.038876471 | 8.866836364 |
| ILMN_1255510 | LMNA | 8.915643333 | 9.554178947 | 9.570432353 | 9.583027273 |
| ILMN_1230546 | CLIC4 | 10.53869333 | 11.17695526 | 11.15126618 | 11.20304091 |
| ILMN_2724570 | MAPK12 | 7.503686667 | 6.865684211 | 6.859533824 | 6.8399 |
| ILMN_2977791 | MAN2A2 | 7.365183333 | 6.727613158 | 6.778547059 | 6.671459091 |
| ILMN_2722784 | CD3G | 11.37494 | 10.73794474 | 10.74789559 | 10.50351818 |
| ILMN_3137291 | LIF | 5.86542 | 6.499805263 | 6.54505 | 6.506213636 |
| ILMN_2983525 | D12ERTD553E | 9.794036667 | 9.160018421 | 9.136989706 | 9.133836364 |
| ILMN_1243249 | 2810410A03RIK | 10.7807 | 10.14750789 | 10.12662647 | 10.07005 |
| ILMN_3141048 | SEPP1 | 7.432253333 | 6.799544737 | 6.843607353 | 6.697863636 |
| ILMN_2769918 | TIMP1 | 5.969726667 | 6.602010526 | 6.577436765 | 6.519463636 |
| ILMN_1237567 | IL7R | 6.91826 | 6.286176316 | 6.243760294 | 6.260904545 |
| ILMN_1257803 | A130095K04RIK | 6.768486667 | 6.137434211 | 6.146425 | 6.027690909 |
| ILMN_1241171 | ANXA3 | 8.77009 | 8.140389474 | 8.239335294 | 8.327272727 |
| ILMN_2993314 | CLEC4N | 8.155996667 | 7.526681579 | 7.760479412 | 8.076522727 |
| ILMN_1231204 | LOC270152 | 7.684566667 | 8.313434211 | 8.328936765 | 8.274381818 |
| ILMN_2758878 | TMEM66 | 8.604496667 | 7.976713158 | 8.059894118 | 7.877477273 |
| ILMN_2661185 | SCML4 | 9.284993333 | 8.657247368 | 8.584035294 | 8.341754545 |
| ILMN_1239346 | KLRE1 | 6.16771 | 6.795257895 | 6.804683824 | 6.700913636 |
| ILMN_2520264 | 2010016118RIK | 8.114473333 | 7.487081579 | 7.539891176 | 7.383822727 |
| ILMN_3014674 | FAM169B | 11.03242333 | 10.40547895 | 10.41317206 | 10.31021364 |
| ILMN_2743902 | MATK | 7.576503333 | 6.950942105 | 6.954210294 | 6.817622727 |
| ILMN_2757807 | BC031353 | 9.67076 | 9.045855263 | 9.010575 | 9.019727273 |
| ILMN_1236245 | RAB11FIP4 | 7.012513333 | 6.387965789 | 6.419583824 | 6.470972727 |
| ILMN_2591342 | BC021614 | 8.086576667 | 7.463065789 | 7.519083824 | 7.435113636 |
| ILMN_2697415 | CD3D | 12.02597333 | 11.40273947 | 11.44940588 | 11.19695909 |
| ILMN_2581681 | D530007E13RIK | 9.03998 | 8.417405263 | 8.385516176 | 8.304936364 |
| ILMN_2601453 | A130092J06RIK | 11.47359667 | 10.85129211 | 10.81008529 | 10.56360455 |
| ILMN_2642913 | EMP1 | 7.33474 | 7.956981579 | 7.96035 | 7.996531818 |
| ILMN_2524865 | CHI3L4 | 8.01563 | 7.393626316 | 7.45365 | 7.612545455 |
| ILMN_1256219 | LOC100047261 | 11.70327667 | 11.08238158 | 11.13535147 | 11.07144545 |
| ILMN_2903169 | GM484 | 6.439876667 | 7.060573684 | 7.045426471 | 7.021390909 |
| ILMN_3004864 | UNC84B | 11.49079 | 10.87096579 | 10.87125294 | 10.75787727 |
| ILMN_2648669 | GPNMB | 7.951466667 | 7.331673684 | 7.401155882 | 7.527768182 |
| ILMN_1216313 | STIM1 | 8.977736667 | 8.359431579 | 8.348152941 | 8.196377273 |
| ILMN_3139253 | BTBD11 | 7.22281 | 6.604578947 | 6.573738235 | 6.500940909 |
| ILMN_2728270 | B4GALNT1 | 10.79857333 | 10.18042632 | 10.25875294 | 10.24815 |
| ILMN_1236507 | 5830496L11RIK | 10.00914667 | 9.393421053 | 9.308585294 | 9.088395455 |
| ILMN_1221820 | FAIM3 | 14.32124333 | 13.70555526 | 13.69330147 | 13.7856 |
| ILMN_2766604 | CAMP | 8.722323333 | 8.106676316 | 8.175870588 | 8.264754545 |
| ILMN_2444984 | MACF1 | 8.425146667 | 7.809781579 | 7.706641176 | 7.701818182 |
| ILMN_3131679 | USP18 | 9.363286667 | 9.978313158 | 9.931382353 | 9.806536364 |
| ILMN_2692615 | TGM2 | 7.39533 | 8.009855263 | 8.055310294 | 8.010068182 |
| ILMN_2449150 | MAP4K2 | 9.819663333 | 9.205721053 | 9.206395588 | 9.199613636 |
| ILMN_1249975 | IGHG | 9.61923 | 9.005610526 | 8.89495 | 8.797990909 |
| ILMN_2571414 | NDR3 | 6.860516667 | 6.247115789 | 6.288842647 | 6.190795455 |
| ILMN_2923865 | CASP6 | 8.833286667 | 9.446118421 | 9.522104412 | 9.610272727 |
| ILMN_3121891 | LMNA | 7.756683333 | 8.369328947 | 8.354533824 | 8.464245455 |
| ILMN_2619408 | ATF3 | 6.142813333 | 6.755302632 | 6.790607353 | 6.803554545 |
| ILMN_2964185 | H2-M2 | 7.242236667 | 6.630168421 | 6.649022059 | 6.640009091 |
| ILMN_2685393 | CCR5 | 6.59509 | 7.20655 | 7.201767647 | 7.132563636 |
| ILMN_2856926 | GPR114 | 9.017556667 | 9.628647368 | 9.590291176 | 9.239136364 |
| ILMN_1221311 | TTYH3 | 8.409613333 | 7.799102632 | 7.858344118 | 7.778395455 |
| ILMN_3034877 | KIF1B | 7.821043333 | 7.211271053 | 7.208664706 | 7.2898 |
| ILMN_1258587 | LOC100044376 | 9.9625 | 9.352865789 | 9.335433824 | 9.182454545 |
| ILMN_1218717 | LOC385615 | 8.83002 | 8.220628947 | 8.213408824 | 7.985740909 |
| ILMN_2594521 | NSDHL | 7.481536667 | 8.090739474 | 8.077933824 | 8.035827273 |
| ILMN_2919411 | OSM | 9.0134 | 9.622018421 | 9.631194118 | 9.557322727 |
| ILMN_2573826 | GAD1 | 6.285003333 | 6.893271053 | 6.891225 | 6.819222727 |
| ILMN_1253354 | DAPP1 | 7.88621 | 8.494418421 | 8.474091176 | 8.498481818 |
| ILMN_2476452 | CPM | 7.303943333 | 6.69575 | 6.678776471 | 6.617622727 |
| ILMN_2834573 | BRWD1 | 8.29335 | 7.685668421 | 7.683051471 | 7.642636364 |
| ILMN_2591754 | QPRT | 7.878863333 | 7.271292105 | 7.245197059 | 7.181613636 |
| ILMN_2738433 | IL4RA | 6.239963333 | 6.846697368 | 6.896517647 | 6.838059091 |
| ILMN_2695158 | KIFC2 | 7.343773333 | 6.737642105 | 6.732835294 | 6.713495455 |
| ILMN_2685392 | CCR5 | 6.51683 | 7.122668421 | 7.051832353 | 7.004754545 |
| ILMN_1229746 | ECM1 | 6.132986667 | 6.738778947 | 6.806132353 | 6.615786364 |
| ILMN_2984744 | EMP3 | 12.08539 | 11.47984474 | 11.51957794 | 11.52749091 |
| ILMN_2652500 | LRG1 | 6.716696667 | 6.111805263 | 6.120491176 | 6.114954545 |
| ILMN_2518483 | PLA2G12A | 6.280293333 | 6.883357895 | 6.955323529 | 6.928695455 |
| ILMN_2845272 | KLHDC2 | 11.45535 | 12.05764211 | 12.05591029 | 12.03670455 |
| ILMN_3105936 | CETN4 | 6.688636667 | 7.2909 | 7.333645588 | 7.254654545 |
| ILMN_3117876 | CHI3L3 | 8.021693333 | 7.419489474 | 7.418464706 | 7.543572727 |
| ILMN_2971171 | FCRLA | 11.14227667 | 10.54146579 | 10.48822647 | 10.40451818 |
| ILMN_1225132 | 4930519L02RIK | 6.88778 | 7.488065789 | 7.525252941 | 7.638254545 |
| ILMN_2774410 | STFA1 | 8.846636667 | 8.247494737 | 8.3801 | 8.518636364 |
| ILMN_3002095 | IL27RA | 11.1748 | 10.57675 | 10.51693824 | 10.35572273 |
| ILMN_2543417 | AUH | 7.760993333 | 8.358818421 | 8.450914706 | 8.496777273 |
| ILMN_1257771 | LOC638301 | 10.25393667 | 9.656502632 | 9.600141176 | 9.438890909 |
| ILMN_2692960 | ERO1LB | 9.701216667 | 10.29802105 | 10.31935588 | 10.34075909 |
| ILMN_2673233 | PFKP | 7.466366667 | 8.062705263 | 8.096848529 | 7.994904545 |
| ILMN_1244891 | CST7 | 7.014076667 | 7.610355263 | 7.708827941 | 7.532645455 |
| ILMN_2651886 | genericK1 | 10.80276 | 11.39885 | 11.35615735 | 11.28045455 |
| ILMN_1228917 | C330023M02RIK | 9.177703333 | 9.773615789 | 9.762547059 | 9.792254545 |
| ILMN_3135781 | ANXA3 | 8.453543333 | 7.858018421 | 7.964625 | 8.134063636 |
| ILMN_2454823 | 6720418B01RIK | 8.454683333 | 7.859492105 | 7.843507353 | 7.601404545 |
| ILMN_1244853 | LOC100044948 | 10.73101 | 11.32439474 | 11.35923971 | 11.16399545 |
| ILMN_1254577 | AI607873 | 6.892946667 | 7.486244737 | 7.533180882 | 7.504613636 |
| ILMN_1222543 | UGT1A10 | 6.93978 | 6.34705 | 6.38325 | 6.614104545 |
| ILMN_2458765 | AHNAK | 9.03018 | 8.437463158 | 8.463539706 | 8.512445455 |
| ILMN_2777696 | GCAT | 8.453923333 | 9.046523684 | 8.955386765 | 8.900027273 |
| ILMN_2591156 | IL27RA | 9.22076 | 8.628213158 | 8.582977941 | 8.52225 |
| ILMN_2676543 | GRIFIN | 6.82902 | 6.237215789 | 6.233313235 | 6.192986364 |
| ILMN_2589401 | PLTP | 6.6056 | 7.196813158 | 7.226439706 | 7.17085 |
| ILMN_2641360 | AK2 | 9.80652 | 10.39720263 | 10.39346912 | 10.44981818 |
| ILMN_2678431 | MNS1 | 7.623163333 | 8.211194737 | 8.219722059 | 8.216736364 |
| ILMN_1252496 | WDR9 | 8.256553333 | 7.668823684 | 7.624570588 | 7.593372727 |
| ILMN_2919259 | SCD2 | 6.989606667 | 7.577144737 | 7.493097059 | 7.432013636 |
| ILMN_2960114 | CYP27A1 | 7.016623333 | 6.429744737 | 6.441141176 | 6.38235 |
| ILMN_2628178 | SOCS2 | 9.017703333 | 9.604081579 | 9.548186765 | 9.538022727 |
| ILMN_2716511 | SLC12A6 | 10.52652333 | 9.940578947 | 9.952173529 | 9.941263636 |
| ILMN_2609813 | CHI3L1 | 9.720116667 | 9.134286842 | 9.338616176 | 9.407072727 |
| ILMN_1226514 | GAB3 | 8.101403333 | 7.515847368 | 7.562842647 | 7.41685 |
| ILMN_2699531 | RGS10 | 10.51234667 | 9.926992105 | 9.937233824 | 9.797122727 |
| ILMN_1217899 | ITK | 6.49019 | 7.075144737 | 7.055417647 | 7.021372727 |
| ILMN_2964986 | CREM | 6.63287 | 7.216378947 | 7.199739706 | 7.17265 |
| ILMN_1245300 | F730045P10RIK | 7.17087 | 7.754107895 | 7.741594118 | 7.777690909 |
| ILMN_3007862 | ABHD14B | 8.3503 | 7.767239474 | 7.744358824 | 7.750127273 |
| ILMN_1222036 | PAQR7 | 7.079483333 | 6.496497368 | 6.448283824 | 6.485727273 |
| ILMN_1214998 | HSD11B1 | 8.95546 | 8.372778947 | 8.421463235 | 8.356695455 |
| ILMN_2645275 | MVD | 8.810396667 | 9.392594737 | 9.422547059 | 9.527990909 |
| ILMN_1217102 | TPCN1 | 9.111683333 | 8.529544737 | 8.543391176 | 8.474540909 |
| ILMN_2692554 | 9330186A19RIK | 7.414916667 | 6.833423684 | 6.748882353 | 6.802618182 |
| ILMN_2605858 | AI646023 | 6.66345 | 6.082005263 | 6.04915 | 6.055072727 |
| ILMN_2652857 | IFI47 | 11.22192333 | 11.80331316 | 11.72213676 | 11.57583636 |
| ILMN_1257444 | SLC37A1 | 7.32784 | 7.907944737 | 7.907548529 | 7.987254545 |
| ILMN_2450767 | DUSP4 | 8.0951 | 8.675071053 | 8.696419118 | 8.827554545 |
| ILMN_2611450 | IFT172 | 9.435683333 | 8.855721053 | 8.856341176 | 8.830854545 |
| ILMN_1242769 | AKAP8L | 10.58592 | 10.00616053 | 9.970663235 | 9.833063636 |
| ILMN_2756438 | D7BWG0611E | 7.023296667 | 6.444452632 | 6.424191176 | 6.389677273 |
| ILMN_1219017 | 5031436O03RIK | 8.368066667 | 7.789578947 | 7.810842647 | 7.816740909 |
| ILMN_2493030 | 2310043N10RIK | 9.993046667 | 9.414568421 | 9.383005882 | 9.301872727 |
| ILMN_1217913 | D230007K08RIK | 9.815053333 | 9.236857895 | 9.225373529 | 8.991240909 |
| ILMN_1256430 | MCOLN3 | 7.33355 | 6.755771053 | 6.748322059 | 6.62405 |
| ILMN_2650255 | MAN2A2 | 7.403953333 | 6.826876316 | 6.818861765 | 6.736995455 |
| ILMN_1248714 | CD55 | 8.813533333 | 8.236618421 | 8.2139 | 8.190377273 |
| ILMN_2740628 | NDRG3 | 7.83159 | 7.255418421 | 7.260383824 | 7.269290909 |
| ILMN_2761109 | CLIC4 | 11.06524333 | 11.64044474 | 11.625275 | 11.63201364 |
| ILMN_1230605 | GM336 | 7.11325 | 7.687934211 | 7.6587 | 7.673177273 |
| ILMN_1219860 | LTK | 8.006776667 | 7.432202632 | 7.398382353 | 7.413186364 |
| ILMN_2810882 | PPIC | 9.265503333 | 8.691218421 | 8.651352941 | 8.542872727 |
| ILMN_2807335 | 3110001A13RIK | 9.544203333 | 8.970439474 | 8.958279412 | 8.976627273 |
| ILMN_2504447 | EG330070 | 7.050706667 | 6.477302632 | 6.498975 | 6.470545455 |
| ILMN_2864309 | OTTMUSG00000000971 | 8.49457 | 7.921613158 | 7.999452941 | 8.111331818 |
| ILMN_1234020 | SCL0002007.1_97 | 9.443223333 | 8.870315789 | 8.903952941 | 8.853154545 |
| ILMN_1228213 | IFI30 | 11.57212333 | 12.14438947 | 12.18978676 | 12.1859 |
| ILMN_1247199 | LOC100046770 | 8.874063333 | 8.301918421 | 8.328780882 | 8.228481818 |
| ILMN_3077377 | WARS | 7.11178 | 7.6835 | 7.700954412 | 7.649859091 |
| ILMN_1252295 | LOC100038894 | 6.395856667 | 6.966326316 | 6.988614706 | 6.970359091 |
| ILMN_2644350 | THY1 | 10.65563333 | 11.22597105 | 11.23514412 | 11.07841364 |
| ILMN_1253008 | ACAT2 | 8.73794 | 9.308271053 | 9.341198529 | 9.285022727 |
| ILMN_2629112 | ASAH3L | 6.420043333 | 6.989957895 | 6.995489706 | 6.845595455 |
| ILMN_2693679 | CCDC125 | 7.619536667 | 7.050602632 | 7.065383824 | 7.088504545 |
| ILMN_3149776 | B3GNT8 | 7.61935 | 7.050542105 | 7.034622059 | 7.055154545 |
| ILMN_2823778 | SC4MOL | 9.339616667 | 9.908247368 | 9.800502941 | 9.942090909 |
| ILMN_2733185 | CDC42EP3 | 8.85545 | 8.286834211 | 8.258167647 | 8.201140909 |
| ILMN_2725402 | NSDHL | 7.48332 | 8.050342105 | 8.035323529 | 8.0115 |
| ILMN_2833248 | C330023M02RIK | 8.10134 | 8.667568421 | 8.672432353 | 8.655227273 |
| ILMN_2774160 | HSD11B1 | 8.706833333 | 8.140992105 | 8.175951471 | 8.124913636 |
| ILMN_2711948 | LOC100047619 | 10.43146667 | 10.99716842 | 10.94898088 | 11.03114545 |
| ILMN_2763739 | BC032203 | 10.80728 | 10.24174474 | 10.17495441 | 10.13608636 |
| ILMN_2593554 | IGTP | 12.18893 | 12.75399474 | 12.77557059 | 12.59678636 |
| ILMN_2733733 | TLR2 | 8.649903333 | 8.085921053 | 8.190616176 | 8.354554545 |
| ILMN_2605453 | RANBP10 | 10.22959667 | 9.666002632 | 9.65415 | 9.647813636 |
| ILMN_2919263 | SLC25A1 | 9.166143333 | 9.728544737 | 9.787044118 | 9.847804545 |
| ILMN_1220397 | TTC28 | 7.667103333 | 7.1052 | 7.127416176 | 7.132168182 |
| ILMN_1249740 | TSSC6 | 7.490173333 | 6.929126316 | 6.912072059 | 6.813518182 |
| ILMN_1245754 | CD84 | 7.52019 | 6.959386842 | 6.929688235 | 6.729927273 |
| ILMN_2930552 | 9130211I03RIK | 6.768166667 | 7.327544737 | 7.359879412 | 7.235159091 |
| ILMN_2698519 | HIST1H3H | 7.513023333 | 8.071597368 | 8.014075 | 8.023959091 |
| ILMN_2721571 | SLAMF1 | 8.52409 | 9.082347368 | 9.069436765 | 8.967859091 |
| ILMN_2900462 | ATP2A3 | 10.93327 | 10.37553947 | 10.34407206 | 10.38361364 |
| ILMN_2883267 | LRRK2 | 9.364593333 | 9.921984211 | 9.942058824 | 9.879545455 |
| ILMN_1248651 | C330023M02RIK | 8.314133333 | 8.871386842 | 8.864563235 | 8.889795455 |
| ILMN_2531737 | LOC240672 | 7.56991 | 8.125805263 | 8.094517647 | 8.219213636 |
| ILMN_1215796 | LOC100046608 | 6.529113333 | 7.084997368 | 7.035101471 | 6.976813636 |
| ILMN_2703563 | STAC2 | 7.38922 | 6.834084211 | 6.875988235 | 6.836763636 |
| ILMN_2599719 | SLC44A2 | 11.92352 | 11.36847105 | 11.35597647 | 11.30884091 |
| ILMN_1227570 | LOC386545 | 8.743896667 | 8.189294737 | 8.230036765 | 7.990959091 |
| ILMN_1222821 | ROGDI | 8.786446667 | 8.232284211 | 8.291411765 | 8.395709091 |
| ILMN_2493826 | UGT1A10 | 7.023866667 | 6.470215789 | 6.490170588 | 6.716004545 |
| ILMN_1230143 | BATF2 | 5.85682 | 6.409157895 | 6.445595588 | 6.382068182 |
| ILMN_1229193 | MSN | 10.28875 | 9.737642105 | 9.735795588 | 9.645190909 |
| ILMN_2483811 | 2210408F11RIK | 11.01110667 | 10.46008421 | 10.42315588 | 10.34929091 |
| ILMN_2660837 | ST6GALNAC4 | 7.92546 | 8.476447368 | 8.480754412 | 8.539186364 |
| ILMN_2767918 | IFI30 | 10.28789667 | 10.83849737 | 10.96806324 | 11.00708182 |
| ILMN_2725414 | CD9 | 9.422196667 | 8.872436842 | 8.904213235 | 8.970577273 |
| ILMN_1231447 | TPI1 | 7.384893333 | 7.934492105 | 7.937911765 | 7.839004545 |
| ILMN_2700354 | DENND5B | 8.391203333 | 7.842021053 | 7.911129412 | 7.785286364 |
| ILMN_1224945 | E130113K22RIK | 8.219303333 | 7.670147368 | 7.685198529 | 7.493963636 |
| ILMN_1226755 | E030007N04RIK | 9.077833333 | 8.528928947 | 8.625920588 | 8.525272727 |
| ILMN_1225214 | CDKN2D | 8.793143333 | 8.244407895 | 8.240169118 | 8.105931818 |
| ILMN_1212982 | ZFP318 | 10.27203 | 9.723307895 | 9.703748529 | 9.6047 |
| ILMN_1254218 | NISCH | 10.79873 | 10.25001842 | 10.28934265 | 10.102 |
| ILMN_2474052 | 5830411I20 | 9.479836667 | 10.02834474 | 10.04819559 | 9.973677273 |
| ILMN_1226901 | MAN2A2 | 7.381073333 | 6.832571053 | 6.870908824 | 6.774418182 |
| ILMN_2684600 | NALP6 | 5.683113333 | 6.230942105 | 6.205075 | 6.286981818 |
| ILMN_1217408 | PRKACB | 12.42386 | 11.87606842 | 11.88194706 | 11.79191818 |
| ILMN_1241610 | ADRB2 | 9.541823333 | 8.994415789 | 8.973683824 | 8.949086364 |
| ILMN_2928599 | SIGLECH | 7.47144 | 6.924655263 | 6.936369118 | 6.803009091 |
| ILMN_2712867 | TIMP2 | 7.230993333 | 6.684518421 | 6.748505882 | 6.6003 |
| ILMN_2593787 | KCNK13 | 5.92443 | 6.470689474 | 6.479725 | 6.553636364 |
| ILMN_1243150 | A130010J15RIK | 9.42285 | 8.876610526 | 8.875494118 | 8.746018182 |
| ILMN_2957862 | NOC4L | 9.10952 | 9.6555 | 9.677673529 | 9.723059091 |
| ILMN_2846865 | ACTB | 10.15901333 | 9.613102632 | 9.645202941 | 9.835927273 |
| ILMN_1218799 | EMB | 10.10022333 | 9.554318421 | 9.501319118 | 9.343713636 |
| ILMN_2054914 | USP18 | 7.713743333 | 8.259573684 | 8.224657353 | 8.138522727 |
| ILMN_1238547 | AREG | 6.01426 | 6.559955263 | 6.514355882 | 6.508963636 |
| ILMN_2711172 | IRGM1 | 6.705733333 | 7.251423684 | 7.156748529 | 7.172445455 |
| ILMN_2845080 | PSAP | 12.3915 | 11.84617895 | 11.88253382 | 11.91787727 |
| ILMN_1239411 | 6430510M02RIK | 9.146923333 | 8.603228947 | 8.535841176 | 8.503222727 |
| ILMN_3079919 | AHNAK | 7.987826667 | 7.444255263 | 7.509125 | 7.546490909 |
| ILMN_1251984 | C730026J16 | 8.910436667 | 9.453307895 | 9.458835294 | 9.488 |
| ILMN_1242794 | D630014A15RIK | 8.913583333 | 8.370760526 | 8.378547059 | 8.273154545 |
| ILMN_2163572 | ADCY7 | 8.611366667 | 8.068673684 | 8.002292647 | 7.965472727 |
| ILMN_2537948 | LOC624610 | 8.479553333 | 9.02185 | 8.960245588 | 8.972936364 |
| ILMN_1246895 | C78339 | 10.46701333 | 9.924855263 | 9.913516176 | 9.792804545 |
| ILMN_1249014 | TBX6 | 7.509416667 | 6.967384211 | 6.934380882 | 6.88865 |
| ILMN_2988931 | STFA1 | 8.375453333 | 7.83345 | 7.962795588 | 8.1119 |
| ILMN_2863390 | FCER1A | 6.565336667 | 7.107023684 | 7.149798529 | 7.251063636 |
| ILMN_1257107 | LOC100043821 | 11.29150333 | 10.74987368 | 10.77962353 | 10.63337727 |
| ILMN_1228937 | CYHR1 | 8.615836667 | 8.074321053 | 8.140413235 | 8.088863636 |
| ILMN_2729958 | HIST1H3D | 9.276753333 | 9.817342105 | 9.843720588 | 9.779963636 |
| ILMN_2664726 | 3110013H01RIK | 7.524233333 | 8.064392105 | 8.045011765 | 8.076427273 |
| ILMN_2662387 | LMNA | 7.710816667 | 8.250452632 | 8.270158824 | 8.388381818 |
| ILMN_2944601 | 4933439C20RIK | 10.40494 | 9.865452632 | 9.853114706 | 9.903972727 |
| ILMN_1224473 | LOC380797 | 8.568663333 | 8.029181579 | 8.096419118 | 7.870022727 |
| ILMN_1225085 | PRKCB | 11.67589 | 11.13770789 | 11.13858382 | 11.04082273 |
| ILMN_2604029 | KLF2 | 11.83327 | 11.29523421 | 11.33738088 | 11.20542727 |
| ILMN_2443164 | SCL0002069.1_48 | 7.571263333 | 8.109255263 | 8.113289706 | 8.152909091 |
| ILMN_1227907 | GMFG | 11.43036667 | 10.89250526 | 10.94621176 | 10.78452727 |
| ILMN_1225764 | MFSD2 | 7.970076667 | 8.507844737 | 8.490097059 | 8.5117 |
| ILMN_2967266 | FXYD5 | 12.00867333 | 11.47132632 | 11.60679559 | 11.49645 |
| ILMN_1236354 | E130302P19RIK | 7.808183333 | 7.271594737 | 7.243542647 | 7.254131818 |
| ILMN_3115796 | CD40 | 11.16950667 | 11.70592105 | 11.66586912 | 11.74807727 |
| ILMN_2597710 | LMNA | 6.945293333 | 7.481457895 | 7.527360294 | 7.643618182 |
| ILMN_1236256 | ARHGEF1 | 10.30117333 | 9.765702632 | 9.777082353 | 9.565368182 |
| ILMN_1231146 | AGFG1 | 10.03638 | 10.57182895 | 10.54505441 | 10.58565 |
| ILMN_2689731 | DUSP4 | 7.44453 | 7.979928947 | 7.960576471 | 8.077036364 |
| ILMN_2476329 | WHRN | 8.198216667 | 7.662876316 | 7.624944118 | 7.688081818 |
| ILMN_3149680 | FBXL12 | 9.55774 | 9.022781579 | 8.979083824 | 8.911004545 |
| ILMN_2891245 | RHBDL2 | 11.17166667 | 10.63687632 | 10.66231765 | 10.44623636 |
| ILMN_3109491 | 6430527G18RIK | 10.69194667 | 10.15745 | 10.05446765 | 9.996531818 |
| ILMN_1251713 | CAR12 | 6.303143333 | 6.837231579 | 6.808888235 | 6.762877273 |
| ILMN_2516221 | TCRB-V8.2 | 8.713246667 | 8.179173684 | 8.227629412 | 7.963136364 |
| ILMN_1234988 | GAD1 | 6.138176667 | 6.672023684 | 6.655942647 | 6.64945 |
| ILMN_2737713 | EDN1 | 6.211046667 | 6.744673684 | 6.792708824 | 6.801963636 |
| ILMN_1220739 | NSG2 | 6.515793333 | 5.982639474 | 5.980491176 | 5.877881818 |
| ILMN_2615739 | GM459 | 10.43873667 | 9.905744737 | 9.871022059 | 10.01163636 |
| ILMN_1228330 | PLCL2 | 9.758736667 | 9.225823684 | 9.202452941 | 9.242722727 |
| ILMN_3160842 | BC087945 | 11.48216667 | 12.01505526 | 11.98382206 | 11.96960909 |
| ILMN_1249637 | PEG13 | 7.152553333 | 6.619871053 | 6.594275 | 6.661804545 |
| ILMN_2620326 | CYP27A1 | 6.618803333 | 6.086228947 | 6.130180882 | 6.038081818 |
| ILMN_1247823 | LRP8 | 6.931496667 | 7.464068421 | 7.458729412 | 7.48775 |
| ILMN_2722996 | SIRPA | 8.616523333 | 8.084239474 | 8.143004412 | 8.417745455 |
| ILMN_2914010 | DMWD | 6.59818 | 7.130428947 | 7.099555882 | 7.121695455 |
| ILMN_1231858 | FCRL1 | 9.11691 | 8.584847368 | 8.649417647 | 8.543518182 |
| ILMN_2684515 | SRPK3 | 11.26210333 | 10.73063158 | 10.67311765 | 10.63067727 |
| ILMN_3160218 | AMICA1 | 7.86067 | 8.392005263 | 8.426604412 | 8.322131818 |
| ILMN_2469253 | VPREB3 | 8.61418 | 8.083365789 | 8.268382353 | 8.165345455 |
| ILMN_2616989 | KCTD17 | 7.071666667 | 7.602447368 | 7.611333824 | 7.592340909 |
| ILMN_2725595 | THRA | 6.775176667 | 6.244463158 | 6.252422059 | 6.26795 |
| ILMN_1217406 | 1110013L07RIK | 8.25656 | 7.726518421 | 7.709145588 | 7.483686364 |
| ILMN_1233545 | LBH | 9.173483333 | 8.643457895 | 8.585191176 | 8.667568182 |
| ILMN_2502346 | SAMSN1 | 6.83519 | 7.365021053 | 7.424139706 | 7.413554545 |
| ILMN_1245924 | MAP3K8 | 8.41872 | 8.948184211 | 8.970817647 | 8.893895455 |
| ILMN_2760765 | BC021381 | 8.589116667 | 8.059802632 | 8.060226471 | 8.142354545 |
| ILMN_2664686 | CHAF1B | 8.82029 | 9.349602632 | 9.297583824 | 9.275677273 |
| ILMN_2424721 | PDGFA | 6.399903333 | 6.928989474 | 6.853522059 | 6.927486364 |
| ILMN_2502136 | CD40 | 10.06332667 | 10.59213684 | 10.70311912 | 10.74522273 |
| ILMN_3116935 | BTLA | 10.49572 | 9.967563158 | 9.945167647 | 9.868113636 |
| ILMN_2925711 | DUSP6 | 9.465566667 | 8.938113158 | 8.877455882 | 9.070204545 |
| ILMN_2770119 | ZBTB32 | 10.86225333 | 11.38918947 | 11.30878824 | 11.42603182 |
| ILMN_1256702 | S100A10 | 9.221003333 | 8.694873684 | 8.738216176 | 8.63095 |
| ILMN_2833441 | TRAFD1 | 10.66275667 | 11.18868947 | 11.17483971 | 11.16915455 |
| ILMN_2522571 | SETD7 | 8.49144 | 7.965744737 | 7.900425 | 7.745868182 |
| ILMN_2448404 | SCL0002975.1_346 | 8.533736667 | 8.008318421 | 8.116742647 | 7.919968182 |
| ILMN_2561749 | A630072I12RIK | 6.90215 | 6.376881579 | 6.363104412 | 6.256609091 |
| ILMN_2992653 | MSH5 | 7.365693333 | 6.840486842 | 6.806542647 | 6.832295455 |
| ILMN_3068231 | MAX | 7.004783333 | 7.529568421 | 7.488708824 | 7.388690909 |
| ILMN_2775202 | RRAGD | 7.19449 | 7.719044737 | 7.690863235 | 7.569304545 |
| ILMN_1253601 | AACS | 9.945826667 | 10.47036053 | 10.46929412 | 10.52398636 |
| ILMN_2694170 | CD97 | 9.80694 | 9.283168421 | 9.352319118 | 9.337640909 |
| ILMN_1222471 | GMFG | 11.24841667 | 10.72540789 | 10.77340735 | 10.59015 |
| ILMN_2923864 | CASP6 | 8.035723333 | 8.557984211 | 8.620341176 | 8.727013636 |
| ILMN_2497190 | BMF | 6.99474 | 6.472515789 | 6.411789706 | 6.365568182 |
| ILMN_2750062 | ARHGAP24 | 7.191523333 | 6.669510526 | 6.721417647 | 6.661459091 |
| ILMN_2776952 | TMEM55B | 8.84177 | 9.363155263 | 9.356698529 | 9.371772727 |
| ILMN_2880536 | UCK2 | 10.53304667 | 11.05398684 | 11.00229706 | 11.02895909 |
| ILMN_2694175 | CD97 | 9.902043333 | 9.381402632 | 9.428180882 | 9.414463636 |
| ILMN_1253851 | HIST2H2BE | 8.37224 | 8.892865789 | 8.900198529 | 8.836145455 |
| ILMN_2756439 | GRAMD1A | 7.12322 | 6.602613158 | 6.640825 | 6.581772727 |
| ILMN_1214703 | NME7 | 6.773053333 | 6.252663158 | 6.253341176 | 6.174459091 |
| ILMN_3151503 | C130032J12RIK | 8.786306667 | 9.305681579 | 9.304608824 | 9.324440909 |
| ILMN_2621901 | BC004022 | 9.374146667 | 9.893202632 | 9.914832353 | 9.842027273 |
| ILMN_2657685 | AASS | 6.827346667 | 7.346360526 | 7.374670588 | 7.304227273 |
| ILMN_1221568 | CDCA7 | 10.56978333 | 11.08840526 | 11.05373088 | 11.14640455 |
| ILMN_1246270 | GINS2 | 7.822786667 | 8.340892105 | 8.295725 | 8.257509091 |
| ILMN_2688236 | ATP2A3 | 12.58813 | 12.07048684 | 12.02009118 | 11.99795455 |
| ILMN_2792601 | P2RY5 | 10.28267667 | 9.765228947 | 9.736329412 | 9.777036364 |
| ILMN_2760979 | TGFBR2 | 12.03852 | 11.52211316 | 11.50930147 | 11.44098182 |
| ILMN_1213483 | FLCN | 10.64120333 | 10.12503947 | 10.09291765 | 10.15793636 |
| ILMN_2705628 | CLEC4D | 8.82084 | 8.305036842 | 8.482082353 | 8.779268182 |
| ILMN_1214318 | RASGRP1 | 6.667076667 | 7.182584211 | 7.145963235 | 6.966668182 |
| ILMN_2771349 | 0610007P14RIK | 9.340376667 | 9.855694737 | 9.816160294 | 9.903672727 |
| ILMN_2681601 | SLC44A2 | 8.348826667 | 7.834094737 | 7.856692647 | 7.706281818 |
| ILMN_1246609 | RASGRP1 | 11.33855 | 11.85307105 | 11.85594265 | 11.60770455 |
| ILMN_2508626 | PEX11C | 6.693453333 | 6.179115789 | 6.188005882 | 6.216986364 |
| ILMN_2617820 | PPP3CC | 10.23106667 | 10.74528684 | 10.718925 | 10.57838182 |
| ILMN_2433964 | GIGYF1 | 9.174583333 | 8.660778947 | 8.685676471 | 8.544772727 |
| ILMN_1229458 | PRKCB | 9.352536667 | 8.83875 | 8.763423529 | 8.807190909 |
| ILMN_2945694 | HIST1H3A | 8.371906667 | 8.885407895 | 8.898982353 | 8.834895455 |
| ILMN_1231309 | LY9 | 10.57010333 | 10.05668158 | 10.06185147 | 10.05565 |
| ILMN_2510383 | TNFRSF25 | 8.859573333 | 8.346544737 | 8.361252941 | 8.340177273 |
| ILMN_2711075 | MMP9 | 7.191016667 | 6.678302632 | 6.702389706 | 6.812577273 |
| ILMN_1249378 | BHLHB2 | 9.07604 | 9.588647368 | 9.570416176 | 9.578259091 |
| ILMN_2506012 | TRP53INP1 | 10.08522 | 9.572681579 | 9.5451 | 9.469318182 |
| ILMN_2509340 | FOXO1 | 8.654366667 | 8.141878947 | 8.104170588 | 8.069745455 |
| ILMN_1221703 | ABCA7 | 10.62202667 | 10.11 | 10.13834265 | 10.13358636 |
| ILMN_2665087 | KLF1 | 6.577006667 | 7.0889 | 7.095722059 | 7.126468182 |
| ILMN_2689307 | SPNB2 | 8.52137 | 8.010315789 | 8.029977941 | 7.859663636 |
| ILMN_1226261 | FAM158A | 7.96853 | 7.457605263 | 7.488210294 | 7.446190909 |
| ILMN_1245354 | TLR7 | 9.301983333 | 9.812560526 | 9.875805882 | 9.876613636 |
| ILMN_2615672 | PRELID2 | 6.108273333 | 6.618676316 | 6.652044118 | 6.542881818 |
| ILMN_2418957 | 5930418K15RIK | 9.004963333 | 8.495173684 | 8.470373529 | 8.374686364 |
| ILMN_2639012 | CCR6 | 10.53305333 | 10.02367368 | 10.09640441 | 10.09141818 |
| ILMN_1233917 | LOC100041463 | 6.175063333 | 6.684076316 | 6.631039706 | 6.519009091 |
| ILMN_2645208 | ARHGEF3 | 12.13058667 | 11.62189474 | 11.58621324 | 11.50325909 |
| ILMN_1252488 | CLEC2I | 7.829716667 | 7.321136842 | 7.3058 | 7.242727273 |
| ILMN_2925008 | SFT2D2 | 6.48672 | 6.995207895 | 6.920232353 | 6.906186364 |
| ILMN_2421246 | TCRB-V8.3 | 8.367016667 | 7.858584211 | 7.941542647 | 7.684159091 |
| ILMN_1742928 | FXYD5 | 10.26460667 | 9.756181579 | 9.868969118 | 9.726668182 |
| ILMN_2604556 | PDE7A | 7.412516667 | 6.904297368 | 6.839942647 | 6.878745455 |
| ILMN_1260323 | AKR1C18 | 5.69947 | 6.207665789 | 6.194161765 | 6.198459091 |
| ILMN_2883268 | LRRK2 | 9.31324 | 9.821171053 | 9.822963235 | 9.761345455 |
| ILMN_2480682 | TMEM23 | 8.860773333 | 8.353092105 | 8.289407353 | 8.190659091 |
| ILMN_2748164 | HIST1H3F | 8.912953333 | 9.420621053 | 9.416923529 | 9.431840909 |
| ILMN_1224034 | PDE1B | 9.389363333 | 8.881973684 | 8.900580882 | 8.945822727 |
| ILMN_2712075 | LCN2 | 11.52582333 | 11.01917105 | 11.18763088 | 11.47061818 |
| ILMN_3125814 | ACSL6 | 5.60918 | 6.115702632 | 6.077122059 | 6.127881818 |
| ILMN_2790357 | SEMA7A | 8.332696667 | 8.839102632 | 8.841805882 | 8.810909091 |
| ILMN_2416460 | TRBV8_AE000663_T_CELL_RECEPTOR_{—} | 8.769396667 | 8.263507895 | 8.351523529 | 8.056295455 |
| | BETA_VARIABLE_8_270 | | | | |
| ILMN_2568028 | IL2RG | 9.496336667 | 8.990715789 | 9.037976471 | 8.851422727 |
| ILMN_1247377 | MPEG1 | 8.820523333 | 9.325578947 | 9.300376471 | 9.23945 |
| ILMN_3153753 | CACNB3 | 7.98161 | 7.476615789 | 7.583061765 | 7.606531818 |
| ILMN_2588055 | ACTB | 9.934683333 | 9.429810526 | 9.439613235 | 9.614481818 |
| ILMN_2880346 | RRP1B | 8.254853333 | 8.759510526 | 8.699454412 | 8.764309091 |
| ILMN_2540344 | LOC381889 | 7.8252 | 8.329739474 | 8.275372059 | 8.279772727 |
| ILMN_1237625 | FAM113B | 8.187733333 | 7.683394737 | 7.662777941 | 7.734872727 |
| ILMN_1214800 | RNF167 | 9.872936667 | 9.368607895 | 9.405135294 | 9.303831818 |
| ILMN_2519313 | TMOD4 | 8.734043333 | 8.229944737 | 8.188375 | 8.138731818 |
| ILMN_2724545 | SBK | 11.35983333 | 10.85581316 | 10.80280588 | 10.82701818 |
| ILMN_2874816 | CDC14B | 7.573256667 | 7.069355263 | 7.069520588 | 7.048240909 |
| ILMN_1224770 | GPRASP1 | 9.77645 | 9.2726 | 9.234861765 | 9.250504545 |
| ILMN_2681516 | SLC39A6 | 8.814913333 | 9.318736842 | 9.304892647 | 9.30945 |
| ILMN_1237990 | GALE | 6.785393333 | 7.288428947 | 7.329561765 | 7.342213636 |
| ILMN_1228783 | LOC100043986 | 6.91082 | 6.407823684 | 6.473876471 | 6.429927273 |
| ILMN_1237114 | TMEM154 | 8.138643333 | 7.636152632 | 7.585944118 | 7.646022727 |
| ILMN_2828916 | FRMD6 | 8.007456667 | 8.509536842 | 8.570277941 | 8.408954545 |
| ILMN_2824002 | FRAT2 | 7.71096 | 7.208939474 | 7.178638235 | 7.178854545 |
| ILMN_2687744 | RNF167 | 9.199576667 | 8.697613158 | 8.739005882 | 8.606045455 |
| ILMN_2920849 | PIRA4 | 7.654663333 | 7.1527 | 7.232370588 | 7.233395455 |
| ILMN_2718453 | ARID5A | 7.283676667 | 7.785302632 | 7.793682353 | 7.815790909 |
| ILMN_2970532 | MCM10 | 9.424043333 | 9.925539474 | 9.829147059 | 9.9184 |
| ILMN_1235795 | LOC675594 | 7.448003333 | 7.948989474 | 7.910132353 | 7.82655 |
| ILMN_2524667 | 4833427B12RIK | 7.739353333 | 8.240297368 | 8.256905882 | 8.22985 |
| ILMN_2673889 | MCM7 | 8.074 | 8.574697368 | 8.487373529 | 8.401995455 |
| ILMN_1259566 | ST6GALNAC4 | 7.177983333 | 7.678428947 | 7.679841176 | 7.755468182 |
| ILMN_1246473 | CCND3 | 7.324866667 | 7.825297368 | 7.841207353 | 7.725622727 |
| ILMN_2830898 | CDC25B | 7.01245 | 6.512081579 | 6.527039706 | 6.583763636 |
| ILMN_3071764 | 1200015F23RIK | 7.43188 | 7.931826316 | 7.960994118 | 7.896445455 |
| ILMN_2628900 | ITGAE | 6.657026667 | 6.157218421 | 6.219135294 | 6.113486364 |
| ILMN_1224472 | CCL4 | 8.713336667 | 9.212842105 | 9.305651471 | 9.529104545 |
| ILMN_2552490 | 6720463L11RIK | 10.58386333 | 10.08456579 | 10.07292353 | 9.899459091 |
| ILMN_2635132 | FOXP3 | 8.401456667 | 8.900668421 | 8.910723529 | 8.765727273 |
| ILMN_1260585 | STFA2 | 7.616773333 | 7.117684211 | 7.219723529 | 7.385345455 |
| ILMN_2625035 | SFT2D2 | 6.582473333 | 7.08145 | 7.109589706 | 7.160159091 |
| ILMN_1237580 | KLRA3 | 6.114223333 | 6.612936842 | 6.616825 | 6.557209091 |
| ILMN_1244211 | LOC238943 | 7.880973333 | 8.379628947 | 8.359485294 | 8.234081818 |
| ILMN_2811263 | ZXDA | 10.18303333 | 9.684407895 | 9.719638235 | 9.468354545 |
| ILMN_2661971 | GM2A | 11.28908333 | 10.79073421 | 10.82771912 | 10.73904545 |
| ILMN_2730926 | LSM12 | 8.963933333 | 9.462136842 | 9.368045588 | 9.434645455 |
| ILMN_1247942 | MGEA5 | 7.989676667 | 7.492289474 | 7.497663235 | 7.336986364 |
| ILMN_1242024 | SETD4 | 7.181716667 | 6.684497368 | 6.698276471 | 6.780113636 |
| ILMN_2929896 | PBK | 8.14347 | 8.640523684 | 8.593113235 | 8.659018182 |
| ILMN_2476733 | 1200016E24RIK | 8.391553333 | 7.895302632 | 7.857470588 | 7.77645 |
| ILMN_2543929 | 2610036L11RIK | 9.511626667 | 10.00778421 | 10.03502353 | 10.05321364 |
| ILMN_2456216 | 5330403D14RIK | 8.630133333 | 8.134192105 | 8.131176471 | 7.9148 |
| ILMN_2727546 | D930048N14RIK | 8.084543333 | 7.5889 | 7.561372059 | 7.503077273 |
| ILMN_3059476 | SESN1 | 7.46764 | 6.972268421 | 7.001925 | 6.872913636 |
| ILMN_2834379 | TGFBI | 7.914363333 | 7.419384211 | 7.540954412 | 7.770859091 |
| ILMN_1247704 | HMGN3 | 11.16247667 | 11.65745263 | 11.61625 | 11.68214091 |
| ILMN_1249888 | ADCY6 | 7.810496667 | 7.315673684 | 7.367497059 | 7.301754545 |
| ILMN_1229534 | RNF167 | 9.146563333 | 8.651771053 | 8.652933824 | 8.584977273 |
| ILMN_1244123 | SLC38A2 | 10.97275 | 10.47811316 | 10.44468824 | 10.36232273 |
| ILMN_1229957 | IL11RA1 | 8.82848 | 8.334539474 | 8.307539706 | 8.341713636 |
| ILMN_2757966 | CXCL4 | 7.484453333 | 6.99065 | 7.080525 | 7.048609091 |
| ILMN_2915951 | D13ERTD608E | 7.322256667 | 6.828815789 | 6.840588235 | 6.650377273 |
| ILMN_2986605 | PDSS1 | 7.05284 | 7.54625 | 7.506525 | 7.626577273 |
| ILMN_1257631 | APOBEC1 | 8.986086667 | 8.492778947 | 8.517795588 | 8.435559091 |
| ILMN_1256025 | LOC100041137 | 6.884853333 | 6.391676316 | 6.44335 | 6.474904545 |
| ILMN_2750725 | PIGZ | 5.767723333 | 6.260307895 | 6.21195 | 6.156859091 |
| ILMN_2848828 | CEP97 | 7.650426667 | 7.157897368 | 7.149170588 | 7.033681818 |
| ILMN_2730293 | PDE1B | 9.44438 | 8.951897368 | 8.937267647 | 9.015109091 |
| ILMN_2918002 | GBP3 | 10.33665333 | 10.82889474 | 10.80859559 | 10.65228636 |
| ILMN_2428252 | RASA2 | 7.455803333 | 7.947894737 | 7.939733824 | 8.004595455 |
| ILMN_1248837 | TBXA2R | 7.84156 | 7.349494737 | 7.354123529 | 7.182513636 |
| ILMN_2866856 | H2-DMA | 12.52671 | 13.01846053 | 13.07551324 | 13.17113636 |
| ILMN_1226555 | 5430417L22RIK | 9.897846667 | 9.406210526 | 9.400447059 | 9.308795455 |
| ILMN_1212938 | AIF1 | 6.576943333 | 7.068328947 | 7.123913235 | 7.142572727 |
| ILMN_2484707 | TYMS | 9.133346667 | 9.624002632 | 9.627598529 | 9.622622727 |
| ILMN_3148489 | FURIN | 10.46270667 | 10.95335 | 10.90541324 | 10.89725909 |
| ILMN_2808485 | GBP10 | 6.781303333 | 7.271921053 | 7.213405882 | 7.041513636 |
| ILMN_1226143 | RABGEF1 | 10.34694667 | 9.856457895 | 9.845116176 | 9.860609091 |
| ILMN_2684563 | CLDND1 | 8.172773333 | 8.663239474 | 8.625180882 | 8.674740909 |
| ILMN_2670398 | EIF4EBP1 | 9.293236667 | 9.783623684 | 9.711872059 | 9.773036364 |
| ILMN_2977535 | D930015E06RIK | 9.695646667 | 9.205305263 | 9.291123529 | 9.344186364 |
| ILMN_1256257 | LOC100038882 | 10.10456 | 10.59481842 | 10.55595588 | 10.57585 |
| ILMN_2613832 | MGST2 | 9.044613333 | 8.554489474 | 8.546391176 | 8.333404545 |
| ILMN_2646834 | D11LGP2E | 8.081966667 | 8.571726316 | 8.579542647 | 8.546236364 |
| ILMN_1242399 | HIST1H2BC | 6.873676667 | 6.384471053 | 6.383308824 | 6.379063636 |
| ILMN_2428798 | 5031439G07RIK | 8.838863333 | 8.350186842 | 8.323948529 | 8.293472727 |
| ILMN_2644140 | PANK4 | 10.61254 | 10.12393684 | 10.18636176 | 10.05719545 |
| ILMN_3163044 | OLA1 | 8.91342 | 9.401918421 | 9.352347059 | 9.322536364 |
| ILMN_1227814 | SRR | 11.62509 | 11.13730526 | 11.21078088 | 11.00605909 |
| ILMN_2644719 | HMGN3 | 10.94508667 | 11.43285263 | 11.41632206 | 11.48374545 |
| ILMN_2600348 | SQLE | 8.251303333 | 8.738868421 | 8.748764706 | 8.667427273 |
| ILMN_2769971 | LINCR | 7.524023333 | 7.036918421 | 7.068355882 | 6.961972727 |
| ILMN_2630328 | PSMA7 | 11.52430333 | 12.01132105 | 12.01410294 | 12.02497727 |
| ILMN_3006990 | EG622339 | 12.98081 | 13.46777632 | 13.46261471 | 13.36065455 |
| ILMN_2915060 | DGKZ | 9.53046 | 9.043510526 | 9.038536765 | 9.060131818 |
| ILMN_2606693 | INSIG1 | 7.262293333 | 7.748865789 | 7.678260294 | 7.641254545 |
| ILMN_2728118 | RRP12 | 9.71417 | 10.20048684 | 10.15763088 | 10.29602273 |
| ILMN_2683811 | GALE | 6.75883 | 7.245031579 | 7.267663235 | 7.272968182 |
| ILMN_2733524 | NRM | 11.53344 | 11.04740263 | 11.00323971 | 10.99424091 |
| ILMN_2660551 | LAT | 9.573943333 | 9.088315789 | 9.004679412 | 8.757095455 |
| ILMN_1241225 | DCTD | 7.73365 | 8.219268421 | 8.183508824 | 8.209872727 |
| ILMN_1259564 | IIGP2 | 10.87997667 | 11.36546316 | 11.33019118 | 11.20709545 |
| ILMN_1214498 | CYP2D22 | 8.11384 | 7.6284 | 7.634107353 | 7.595440909 |
| ILMN_3148550 | GOLM1 | 8.432583333 | 7.947163158 | 7.913177941 | 7.917890909 |
| ILMN_1260512 | MIF | 11.02496667 | 11.51028421 | 11.51826471 | 11.4299 |
| ILMN_1248389 | INPP5K | 12.04935667 | 11.56430789 | 11.54765294 | 11.48464091 |
| ILMN_2547942 | GINS2 | 7.60201 | 8.087055263 | 8.042544118 | 8.018609091 |
| ILMN_1223257 | CCL4 | 11.14475 | 11.62975263 | 11.75993824 | 11.86287727 |
| ILMN_2622780 | 5530601\|19RIK | 7.999706667 | 7.515147368 | 7.602066176 | 7.569886364 |
| ILMN_2974064 | OSBPL3 | 7.03954 | 7.523976316 | 7.509626471 | 7.605709091 |
| ILMN_1228333 | PRF1 | 6.592786667 | 7.077028947 | 7.063494118 | 6.897177273 |
| ILMN_2915059 | DGKZ | 10.26964333 | 9.785439474 | 9.7872 | 9.816468182 |
| ILMN_3721360 | GALK1 | 9.165806667 | 9.649981579 | 9.677827941 | 9.563363636 |
| ILMN_2608151 | FCRL1 | 9.303546667 | 8.819431579 | 8.865113235 | 8.792713636 |
| ILMN_2417174 | UBE2H | 7.774013333 | 7.291031579 | 7.304219118 | 7.268663636 |
| ILMN_2901283 | ADD3 | 10.85499 | 10.37201579 | 10.37048824 | 10.28376818 |
| ILMN_2508001 | CLTC | 7.302493333 | 7.785318421 | 7.737447059 | 7.817286364 |
| ILMN_2697615 | MAPK12 | 7.052833333 | 6.570236842 | 6.618429412 | 6.654509091 |
| ILMN_1230696 | IGL-V1 | 12.40741667 | 11.92506842 | 12.00315735 | 12.03508182 |
| ILMN_1237978 | 9930005O13RIK | 7.786423333 | 7.304186842 | 7.242588235 | 7.192336364 |
| ILMN_1233141 | KLRA22 | 5.737783333 | 6.220013158 | 6.217479412 | 6.088368182 |
| ILMN_2597255 | CDC6 | 7.808103333 | 8.289960526 | 8.186636765 | 8.23955 |
| ILMN_1214578 | CRTC3 | 8.313713333 | 7.832310526 | 7.893926471 | 7.840259091 |
| ILMN_2878548 | MTHFD2 | 8.144953333 | 8.626257895 | 8.628497059 | 8.6541 |
| ILMN_2671738 | IL16 | 10.34373 | 9.862731579 | 9.813888235 | 9.759777273 |
| ILMN_2755021 | CHKB | 9.912793333 | 9.432428947 | 9.525319118 | 9.472736364 |
| ILMN_1249217 | LOC380763 | 8.500983333 | 8.020868421 | 8.019075 | 7.913895455 |
| ILMN_2623536 | GOLM1 | 8.876253333 | 8.396268421 | 8.368392647 | 8.371622727 |
| ILMN_1257097 | CNP | 8.4583 | 8.937923684 | 8.996055882 | 8.950890909 |
| ILMN_3142573 | MAPK1IP1 | 8.969603333 | 8.490186842 | 8.481775 | 8.445359091 |
| ILMN_1220284 | PSMD7 | 9.257483333 | 9.736878947 | 9.752014706 | 9.74925 |
| ILMN_3067831 | ZFP187 | 9.802603333 | 9.323465789 | 9.313083824 | 9.315436364 |
| ILMN_2665131 | IHPK1 | 10.7729 | 10.29377105 | 10.30605147 | 10.30897273 |
| ILMN_2600744 | RGS16 | 6.451433333 | 6.930547368 | 6.940605882 | 7.104027273 |
| ILMN_1220595 | IL17A | 6.454306667 | 6.933336842 | 6.982905882 | 6.86355 |
| ILMN_2641793 | DTX1 | 9.060943333 | 8.582018421 | 8.668863235 | 8.689840909 |
| ILMN_2880529 | UCK2 | 7.70306 | 8.181926316 | 8.224991176 | 8.167672727 |
| ILMN_2819380 | BC030476 | 11.90582667 | 11.42737895 | 11.43873382 | 11.16817727 |
| ILMN_1232093 | LOC386330 | 10.13935 | 9.660942105 | 9.652973529 | 9.685572727 |
| ILMN_1242013 | UCK2 | 9.413536667 | 9.891402632 | 9.895961765 | 9.882868182 |
| ILMN_2899599 | DDX3Y | 9.722653333 | 9.245560526 | 9.202622059 | 9.122063636 |
| ILMN_1242466 | PSMB9 | 8.003013333 | 8.479313158 | 8.365239706 | 8.325395455 |
| ILMN_2901284 | ADD3 | 11.49975333 | 11.02383684 | 10.98140147 | 10.84233636 |
| ILMN_2589477 | DARS | 8.916623333 | 9.392265789 | 9.302201471 | 9.310081818 |
| ILMN_1255416 | LY6A | 14.19523333 | 14.67078421 | 14.64110735 | 14.66443636 |
| ILMN_1236517 | IL18 | 6.902506667 | 6.427465789 | 6.373588235 | 6.419559091 |
| ILMN_2470564 | IGHV1S120_AF025443_IG_HEAVY_{—} | 9.692856667 | 9.217965789 | 9.226329412 | 9.119081818 |
| | VARIABLE_1S120_8 | | | | |
| ILMN_1240702 | ATP183 | 12.44345333 | 11.96903947 | 11.89987059 | 11.84145455 |
| ILMN_1242046 | AQR | 8.068703333 | 7.594457895 | 7.619404412 | 7.522959091 |
| ILMN_1220972 | IL16 | 7.42725 | 6.953173684 | 6.940511765 | 6.881495455 |
| ILMN_3154810 | SMARCA2 | 11.02438 | 10.55043684 | 10.50354265 | 10.53565909 |
| ILMN_1255664 | LOC100046905 | 7.896743333 | 7.422971053 | 7.463869118 | 7.464240909 |
| ILMN_2951691 | HIST1H3E | 9.41995 | 9.893715789 | 9.930129412 | 9.921827273 |
| ILMN_1247832 | CD74 | 10.73217667 | 11.20577895 | 11.29666471 | 11.35442273 |
| ILMN_2918114 | APEX1 | 8.05855 | 8.53215 | 8.552566176 | 8.5933 |
| ILMN_1216386 | CD86 | 8.408796667 | 8.882105263 | 8.877667647 | 8.874636364 |
| ILMN_2663604 | FIGNL1 | 8.27945 | 8.752171053 | 8.647223529 | 8.705381818 |
| ILMN_2778122 | PDCD4 | 11.32195333 | 10.84939211 | 10.88932794 | 10.78596818 |
| ILMN_2565835 | 8930008G03RIK | 11.46631667 | 10.99378421 | 10.96833235 | 10.91645 |
| ILMN_3020240 | HBP1 | 10.99606333 | 10.52378947 | 10.556525 | 10.49137273 |
| ILMN_1246339 | 6330403E01RIK | 9.15174 | 8.679492105 | 8.702944118 | 8.593131818 |
| ILMN_2467429 | D6MIT97 | 11.45000333 | 10.97782632 | 10.98452206 | 11.02852727 |
| ILMN_1258394 | AARS | 10.25052333 | 10.72222632 | 10.64604118 | 10.8079 |
| ILMN_2804166 | IGSF9 | 7.72168 | 7.250102632 | 7.209426471 | 7.318354545 |
| ILMN_1255750 | IL7R | 6.3964 | 5.925234211 | 5.865013235 | 5.894859091 |
| ILMN_2737710 | TIAM1 | 8.273783333 | 8.744307895 | 8.661008824 | 8.648690909 |
| ILMN_2595814 | A630082K20RIK | 10.71696667 | 10.24649474 | 10.22477353 | 10.16600909 |
| ILMN_1214486 | TK2 | 10.35887333 | 9.888563158 | 9.842289706 | 9.781713636 |
| ILMN_2432255 | LOC630337 | 9.698646667 | 9.228815789 | 9.138663235 | 9.038581818 |
| ILMN_1248439 | ST6GALNAC4 | 8.2337 | 8.703436842 | 8.618572059 | 8.675536364 |
| ILMN_2623056 | CLSPN | 8.064613333 | 8.534094737 | 8.475572059 | 8.56605 |
| ILMN_2979639 | H2-DMB2 | 11.28671667 | 11.75598684 | 11.78886176 | 11.93475455 |
| ILMN_2869312 | FBXO4 | 10.21257 | 10.68178421 | 10.61175 | 10.58898182 |
| ILMN_2513781 | A130038J17RIK | 7.070466667 | 6.601365789 | 6.61995 | 6.578772727 |
| ILMN_1247762 | RASAL1 | 7.21572 | 6.746647368 | 6.816261765 | 6.937468182 |
| ILMN_1232667 | IFITM2 | 10.14822 | 10.6172 | 10.71305735 | 10.73515909 |
| ILMN_2918479 | HSPA8 | 12.4956 | 12.71828947 | 12.60738235 | 12.47838636 |
| ILMN_1251178 | HS3ST3B1 | 7.42913 | 6.960407895 | 6.955142647 | 6.921104545 |
| ILMN_3000080 | 1110038D17RIK | 10.41259667 | 9.943955263 | 9.934307353 | 9.923922727 |
| ILMN_1230599 | ADAM23 | 6.62476 | 6.156160526 | 6.138545588 | 6.24995 |
| ILMN_2644092 | AASS | 7.530816667 | 7.999257895 | 7.906854412 | 8.041690909 |
| ILMN_2668319 | HSP90AA1 | 8.8777 | 9.345960526 | 9.299935294 | 9.217740909 |
| ILMN_2723860 | PKM2 | 8.854423333 | 9.322594737 | 9.358717647 | 9.247309091 |
| ILMN_1214634 | AQP9 | 6.903756667 | 7.371810526 | 7.386388235 | 7.409309091 |
| ILMN_2866185 | BTG1 | 13.71557333 | 13.24755263 | 13.26201324 | 13.26215 |
| ILMN_2660466 | EG433229 | 7.618856667 | 7.150876316 | 7.158276471 | 7.124095455 |
| ILMN_2431398 | 2810001G20RIK | 7.20617 | 6.738457895 | 6.759751471 | 6.705422727 |
| ILMN_2859847 | PYGL | 7.790936667 | 7.3233 | 7.422495588 | 7.578627273 |
| ILMN_2883952 | 1810015A11RIK | 8.104256667 | 8.571594737 | 8.509154412 | 8.564577273 |
| ILMN_1232989 | EHD3 | 8.013176667 | 7.546005263 | 7.554476471 | 7.601840909 |
| ILMN_2499056 | IGKV2-137_AJ231263_IG_KAPPA_{—} | 11.41894333 | 10.95204211 | 10.96605441 | 10.97854091 |
| | VARIABLE_2-137_15 | | | | |
| ILMN_1233116 | MAP4K4 | 7.267023333 | 6.800147368 | 6.785232353 | 6.852222727 |
| ILMN_2477221 | CITED2 | 9.57618 | 10.04302895 | 10.01740882 | 10.04663636 |
| ILMN_2933431 | INPP5K | 9.374156667 | 8.907313158 | 8.987426471 | 8.93495 |
| ILMN_1224942 | SHMT2 | 8.329433333 | 8.795918421 | 8.750304412 | 8.834072727 |
| ILMN_2464474 | 9430029L20RIK | 9.4229 | 8.95645 | 8.990469118 | 8.856822727 |
| ILMN_3130350 | GLIPR2 | 6.38496 | 6.850863158 | 6.813057353 | 6.812659091 |
| ILMN_1225733 | LOC677643 | 10.39900333 | 9.9334 | 9.931026471 | 9.857759091 |
| ILMN_1252202 | TNFAIP3 | 8.73625 | 9.200576316 | 9.205004412 | 9.258704545 |
| ILMN_1252481 | FOSL2 | 7.555466667 | 8.019776316 | 8.056545588 | 8.1423 |
| ILMN_1228653 | ZBP1 | 6.948423333 | 7.412642105 | 7.444173529 | 7.299622727 |
| ILMN_1231168 | H3F3B | 9.362493333 | 8.898360526 | 8.812038235 | 8.781254545 |
| ILMN_2858359 | CLSPN | 8.452636667 | 8.916521053 | 8.857144118 | 8.940954545 |
| ILMN_2574982 | SPN | 7.25949 | 6.7957 | 6.822661765 | 6.698304545 |
| ILMN_2600022 | HEG1 | 8.128633333 | 7.664915789 | 7.681570588 | 7.608509091 |
| ILMN_2593774 | 1190002H23RIK | 8.001166667 | 7.537692105 | 7.583389706 | 7.431922727 |
| ILMN_2892856 | RNF167 | 9.68891 | 9.225528947 | 9.181063235 | 9.116222727 |
| ILMN_2899863 | TNF | 8.296036667 | 8.759357895 | 8.846445588 | 8.912131818 |
| ILMN_2619846 | SLC25A1 | 7.50881 | 7.971526316 | 8.031520588 | 8.062972727 |
| ILMN_2804103 | BC038822 | 7.910843333 | 7.448302632 | 7.379916176 | 7.437740909 |
| ILMN_2469743 | LOC100046793 | 12.45598333 | 11.99356316 | 11.99824265 | 12.05897727 |
| ILMN_2660596 | RUNDC3B | 6.71609 | 7.178071053 | 7.1822 | 7.1.21272727 |
| ILMN_2431619 | UBE2L6 | 8.088856667 | 8.550692105 | 8.561057353 | 8.579322727 |
| ILMN_2688075 | CYP51 | 9.241013333 | 9.702565789 | 9.707444118 | 9.776145455 |
| ILMN_1255256 | SGCB | 6.903226667 | 7.364521053 | 7.292351471 | 7.317954545 |
| ILMN_3133748 | GAB3 | 7.71051 | 7.249381579 | 7.207739706 | 7.064881818 |
| ILMN_2769567 | F2RL1 | 6.789253333 | 6.328434211 | 6.311585294 | 6.318131818 |
| ILMN_2927131 | IL13 | 6.278206667 | 6.738778947 | 6.699602941 | 6.664881818 |
| ILMN_2889832 | SERPINA3H | 7.487796667 | 7.948363158 | 7.94995 | 8.069 |
| ILMN_2793946 | CSNK1G3 | 10.25665333 | 9.796518421 | 9.729845588 | 9.704345455 |
| ILMN_2770917 | BLVRB | 8.54669 | 9.006694737 | 9.003377941 | 8.950313636 |
| ILMN_2631259 | IGK-V5 | 11.67123 | 11.21130789 | 11.21311029 | 11.28002727 |
| ILMN_2784272 | IFNGR2 | 10.17693333 | 9.717152632 | 9.806033824 | 9.636736364 |
| ILMN_2467365 | PEX11C | 6.63978 | 6.1805 | 6.161683824 | 6.187445455 |
| ILMN_3093089 | TARDBP | 8.83852 | 8.379476316 | 8.366308824 | 8.351659091 |
| ILMN_2599018 | CLIC4 | 6.676103333 | 7.134968421 | 7.206091176 | 7.233940909 |
| ILMN_2772920 | FBXO4 | 9.511573333 | 9.970065789 | 9.933588235 | 9.823972727 |
| ILMN_2814847 | SCN4A | 6.732753333 | 7.191044737 | 7.299491176 | 7.273713636 |
| ILMN_1240153 | UCHL5 | 8.30777 | 8.765928947 | 8.739652941 | 8.759190909 |
| ILMN_1250135 | A930005H10RIK | 10.44449 | 9.986555263 | 10.03557794 | 9.894318182 |
| ILMN_2587761 | KIF1B | 6.898756667 | 6.440939474 | 6.393510294 | 6.337227273 |
| ILMN_3026397 | CHKA | 12.51251667 | 12.05482368 | 12.07835147 | 11.87520909 |
| ILMN_2888448 | CCR6 | 11.8719 | 11.41442368 | 11.42346618 | 11.433 |
| ILMN_1258600 | LOC100043671 | 9.537613333 | 9.99505 | 9.958407353 | 10.02151818 |
| ILMN_2917180 | FOXP3 | 7.212343333 | 7.669513158 | 7.709433824 | 7.612072727 |
| ILMN_1248891 | CRTC3 | 8.721226667 | 8.2641 | 8.262083824 | 8.305422727 |
| ILMN_2612448 | NFAT5 | 10.1261 | 9.669371053 | 9.617407353 | 9.650840909 |
| ILMN_3149143 | ENTPD5 | 7.866756667 | 7.41035 | 7.433564706 | 7.419763636 |
| ILMN_1248824 | C230082I21RIK | 9.24562 | 8.789247368 | 8.743510294 | 8.73275 |
| ILMN_1229263 | LOC100046496 | 10.91076 | 10.45446316 | 10.498725 | 10.60142727 |
| ILMN_2677876 | ARHGAP4 | 11.83005333 | 11.37396842 | 11.37664706 | 11.29995909 |
| ILMN_3118584 | BEX4 | 6.290316667 | 6.745934211 | 6.717019118 | 6.743859091 |
| ILMN_2469190 | B230345P09RIK | 9.48775 | 9.032318421 | 8.997217647 | 8.814136364 |
| ILMN_2753149 | AVIL | 5.968846667 | 6.424028947 | 6.406513235 | 6.338922727 |
| ILMN_2514723 | 1110067B18RIK | 8.664656667 | 8.209513158 | 8.227560294 | 8.173459091 |
| ILMN_1239601 | LOC637711 | 7.591996667 | 8.047021053 | 8.053213235 | 8.032568182 |
| ILMN_2686132 | ADA | 7.039233333 | 7.494160526 | 7.524789706 | 7.525754545 |
| ILMN_1235493 | FXYD5 | 10.50235333 | 10.04759737 | 10.05027206 | 9.985063636 |
| ILMN_1244857 | RABAC1 | 11.18026333 | 10.72565526 | 10.79111324 | 10.75775455 |
| ILMN_1257965 | LDHA | 12.56264333 | 13.01716842 | 12.98936765 | 12.88280455 |
| ILMN_2597030 | SMOX | 7.755666667 | 8.210173684 | 8.189514706 | 8.309086364 |
| ILMN_1241923 | MSH5 | 7.286276667 | 6.83185 | 6.818532353 | 6.777359091 |
| ILMN_1239607 | DGKZ | 8.896886667 | 8.442460526 | 8.459808824 | 8.481786364 |
| ILMN_2522495 | C130078N17RIK | 9.92795 | 10.38224737 | 10.34155441 | 10.21982727 |
| ILMN_1232144 | A130087I02RIK | 6.94611 | 6.491957895 | 6.482135294 | 6.369822727 |
| ILMN_2695217 | 2810485I05RIK | 10.85201 | 10.39787632 | 10.37109853 | 10.27317273 |
| ILMN_1249864 | A630077B13RIK | 8.645473333 | 9.099492105 | 9.099207353 | 8.909022727 |
| ILMN_1221943 | SDF2L1 | 9.424183333 | 9.878152632 | 9.842982353 | 10.00285909 |
| ILMN_1218934 | RDM1 | 9.691513333 | 9.237818421 | 9.296355882 | 9.263672727 |
| ILMN_1225932 | CCND2 | 7.983533333 | 8.437152632 | 8.424633824 | 8.339681.818 |
| ILMN_2538029 | LOC386005 | 11.26105 | 10.80766316 | 10.76714265 | 10.68383636 |
| ILMN_2571616 | C430002D13RIK | 8.354466667 | 7.901107895 | 7.951077941 | 7.801709091 |
| ILMN_2891506 | TBCD | 9.07411 | 9.527063158 | 9.515958824 | 9.499972727 |
| ILMN_2516348 | VAMP4 | 8.802203333 | 8.349344737 | 8.311704412 | 8.185272727 |
| ILMN_2624938 | PEA15 | 8.426816667 | 7.973960526 | 8.031760294 | 7.905472727 |
| ILMN_2484838 | UBAC2 | 9.095233333 | 8.642694737 | 8.613807353 | 8.500886364 |
| ILMN_2673369 | IRF8 | 10.46650667 | 10.91894737 | 10.92003235 | 10.8755 |
| ILMN_2663230 | SLCO3A1 | 7.528683333 | 7.980968421 | 8.006858824 | 7.821268182 |
| ILMN_1213645 | AI467606 | 10.8157 | 10.36356316 | 10.381575 | 10.31484545 |
| ILMN_1224390 | 1700129I04RIK | 6.254066667 | 6.706015789 | 6.752007353 | 6.905109091 |
| ILMN_2672091 | HTT | 7.075923333 | 7.527834211 | 7.447980882 | 7.573559091 |
| ILMN_2705097 | DEADC1 | 8.784596667 | 9.236284211 | 9.246120588 | 9.286304545 |
| ILMN_2417863 | TNIP1 | 10.67977667 | 10.22862895 | 10.33654118 | 10.3292 |
| ILMN_2630605 | FSCN1 | 9.264746667 | 8.813610526 | 8.879035294 | 9.026695455 |
| ILMN_2710229 | CCNG1 | 8.237643333 | 8.688307895 | 8.719710294 | 8.740531818 |
| ILMN_2602597 | SH3RF1 | 6.97383 | 7.424310526 | 7.302129412 | 7.306981818 |
| ILMN_1227018 | IL1A | 7.508863333 | 7.959210526 | 8.102714706 | 8.221045455 |
| ILMN_2727235 | ANKRD11 | 10.3901 | 9.939768421 | 9.97785 | 9.793654545 |
| ILMN_2759484 | C3 | 8.713386667 | 8.263144737 | 8.351541176 | 8.556345455 |
| ILMN_2814484 | IFITM1 | 6.603923333 | 7.054015789 | 7.066145588 | 7.122472727 |
| ILMN_2526739 | BATF3 | 6.226883333 | 6.676947368 | 6.730197059 | 6.660731818 |
| ILMN_1215825 | LOC100047316 | 9.10279 | 8.652736842 | 8.764692647 | 8.732336364 |
| ILMN_1219978 | APPL2 | 8.213966667 | 7.764534211 | 7.741592647 | 7.672322727 |
| ILMN_2751948 | HIST2H2AA1 | 7.52977 | 7.080352632 | 7.127307353 | 7.225190909 |
| ILMN_1225769 | CLASP1 | 8.361373333 | 7.912028947 | 7.920219118 | 7.866618182 |
| ILMN_1218547 | DLM1-PENDING | 6.341386667 | 6.79065 | 6.869138235 | 6.909118182 |
| ILMN_2974737 | BC065085 | 7.958643333 | 7.509571053 | 7.581791176 | 7.586068182 |
| ILMN_2655571 | B3GNT7 | 6.033913333 | 6.482823684 | 6.563373529 | 6.687959091 |
| ILMN_2585533 | LY78 | 8.423283333 | 7.974515789 | 7.950780882 | 7.901995455 |
| ILMN_3138439 | DYRK2 | 7.370266667 | 6.921794737 | 6.905360294 | 6.828077273 |
| ILMN_2524861 | CHD3 | 8.387586667 | 7.939173684 | 7.977804412 | 7.910968182 |
| ILMN_2995688 | EG433016 | 9.116706667 | 8.66845 | 8.852829412 | 9.032322727 |
| ILMN_3004142 | STK4 | 11.51383 | 11.06564474 | 11.06916765 | 11.10165 |
| ILMN_1217855 | NKG7 | 9.221053333 | 9.669168421 | 9.689545588 | 9.423554545 |
| ILMN_2543834 | RFC3 | 6.905386667 | 7.353431579 | 7.309782353 | 7.283140909 |
| ILMN_1236304 | HAMP | 5.92571 | 6.373726316 | 6.455057353 | 6.366813636 |
| ILMN_1223697 | CD44 | 9.625953333 | 10.07376842 | 10.11732941 | 10.10371364 |
| ILMN_2700848 | ARRB2 | 9.449236667 | 9.001426316 | 9.048977941 | 9.081027273 |
| ILMN_2949605 | UBAC2 | 10.55435 | 10.10654737 | 10.07797647 | 9.933981818 |
| ILMN_2743503 | E2F3 | 7.260286667 | 7.707805263 | 7.726333824 | 7.720931818 |
| ILMN_2546073 | WDR68 | 7.13092 | 7.577952632 | 7.545673529 | 7.717172727 |
| ILMN_2595973 | GRN | 11.08555667 | 10.63854737 | 10.66243382 | 10.77950909 |

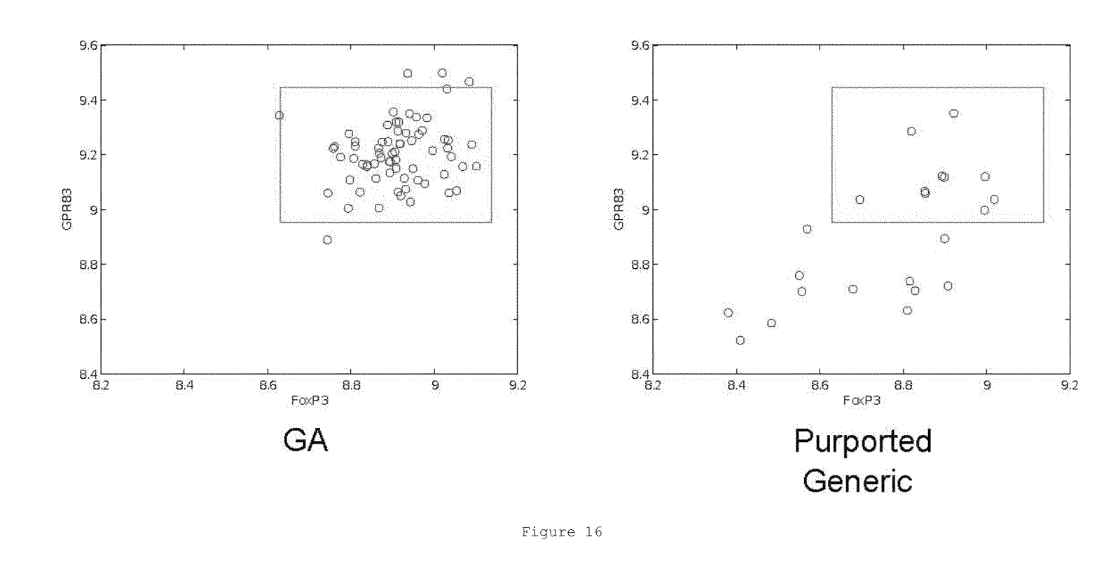

For each of the 1000 probes, the maximum and minimum expression observed by any reference standard sample was recorded. The range between the maximum and minimum expression observed for the reference standard served as the acceptable tolerance range for each probe. We then counted the number of samples for GA and PG with expression that fell within the acceptable tolerance range and converted the results to a percentage of samples within range for all 1000 probes. These percentages were then sorted from smallest to largest separately for PG and GA and plotted against the integers, 1-1000. The net result was a plot that allowed for the determination for either drug of how many probes would fail to meet a given processing specification on the number of samples required to fall within the acceptable tolerance range for each probe. A visual representation of the method can be observed in FIG. 16, which depicts scatter plots for Gpr83 vs. FoxP3 for both GA and PG. The red square in both plots is the acceptable tolerance range defined by the maximum and minimum reference standard expression for both probes. Five probes fall outside the acceptable tolerance range for GA, while twelve probes fall outside the range for PG.

Variance Ratio Method

To measure relative differences in the variability among PG and GA samples, we utilized the sample variance as an unbiased estimate for the population variance for each set of treatments (GA and PG). Briefly, we computed the ratio of the variance in PG samples divided by the variance in the GA samples for each probe in the Illumine microarray. The measure provided an intuitive comparison of the variance in each probe between treatments and this ratio is basic statistic computed by the F-test (63). We then sorted the probes from higher than 1 ratio (more variability in PG compared to GA) to lower than 1 (more variable in GA compared to PG).

Variability Analysis Over Time Plots

To analyze and compare the variability of PG and GA treated samples, we narrowed the list of probes to those with high expression (top 10% highest expression by rank) and a high coefficient of variation (CV, top 10% highest CV by rank). The combined criteria yield a set of 315 probes that are both highly expressed (mean log 2 intensity>=9) and highly variable (mean CV>=2%).

To measure the distance between any two categories/batches, we simply counted the number of differentially variable probes by F-test with FDR-adjusted P<0.05 between those categories, collapsing technical replicates using the mean for each probe to minimize the effects of technical variation (64). We plotted the number of differentially variable probes between each PG batch and the reference standard (RS) and GA. We also noted the percent of probes (out of the total 315) that were differentially variable between GA and RS in the horizontal green line for comparison.

Coefficient of Variation Plots

To measure the relationship between probe intensity and probe variation, we calculated the log 2 intensity of each probe after normalization/batch correction (averaging technical replicates to focus on biological variability rather than variability introduced by technical issues (64). We also calculated the coefficient of variation, denoted CV, by dividing the standard deviation of each probe by its mean log 2 intensity. We then plotted the relationship for all probes using the log 2 intensity of the probe on the X-axis and the CV on the y-axis. The plots show a bias of probes with higher intensity to have lower CV and vice versa. Despite this bias, we still see that, as a class, PG-treated samples exhibit overall significantly higher variability (measure by F-test) compared to GA treated samples based on the number of probes that were highly variable between the two treatments and medium.

Identification of Differentially Expressed Genes Using Multiple Parametric Tests (ANOVA, LIMMA with Background Subtraction, Comparative Marker Selection with SNR and T-Test)

To find differentially expressed probes between PG and GA, we utilized various statistical tests at the probe level and merged the results across the different methods. First, we computed the statistical significance of differential expression between treatments using the Analysis of Variance (ANOVA) method for each probe (65), adjusting for multiple hypothesis testing using the Benjamini-Hochberg False Discovery Rate (FDR) correction (66). Next, we utilized Linear Models for Microarray (LIMMA) data analysis (67, 68) R package, part of the Bioconductor framework (69), to compare PG and GA samples, fitting a linear model that adjusts for fixed effect from medium (Effect=(GA-PG)−(PG-Medium)). The coefficients for the linear model were tested for significance using a modified t-test (taking into account standard deviation) and the p-values for each probe were adjusted using FDR. (66) In parallel, we used Comparative Marker Selection as implemented in GenePattern (70) to directly compare probes between PG and GA. We applied two separate techniques within this framework; a traditional T-test and a Signal-to-Noise Ratio test (SNR). For each of these two tests, we adjusted the nominal p-values via FDR. For all four tests described, we used an adjusted threshold that was less than or equal to 0.05.

Non-Parametric/Wilcoxon

Because of natural variations of the distributions of probe expression in samples of PG and GA, we sought to identify differences via a non-parametric approach. For this, we used the Wilcoxon Rank Sum Test (71) as implemented in R (R version 2.15.1 (2012-06-22)) for each probe.

Nominal p-values were FDR adjusted and only probes that were less than or equal to 0.05 were considered.

GSEA with FoxP3 Target Gene Lists

In order to examine how genes downstream of the FoxP3 transcription factor were modulated by the two treatments, we utilized gene sets constructed by Zheng et al. (72) from FoxP3+ T-cells isolated from human thymus and periphery comprising genes that had FoxP3 binding sites by ChIP (Chromatin-Immuniprecipitation) and are differentially expressed relative to FoxP3-T-cells. Orthology mapping from human to mouse was conducted using a map provided by the Mouse Genome Database. (73) We then utilized the Gene Set Enrichment Algorithm (GSEA) (48) to measure the enrichment of FoxP3 targets in genes upregulated in GA relative to Medium, and PG relative to Medium. Briefly, GSEA takes as input a gene set (in this case, the set of FoxP3 targets) and an expression matrix (the set of samples treated with either PG or GA or untreated/Medium), then it ranks genes based on their expression in the expression matrix for each class/treatment. GSEA then calculates an enrichment score for each geneset based on how overrepresented each geneset is at the extremes of expression (high or low expression) for each treatment.

ANOVA-Based Pattern Identification Method:

We sought to identify probes matching specific patterns of expression across experimental conditions using a technique that referred to as the ANOVA-based Pattern Identification Method.

For instance, one pattern of interest was where probes are only significantly affected by PG. In this pattern, the expression for a given probe should not be statistically different among cells treated by GA, reference standard, or Medium. In statistical terms, probes matching this pattern should have p-values for the comparisons between PG and GA (pGA-PG), PG and reference standard (pPG-reference standard), and PG and Medium (pPG-Medium) less than 0.05 and p-values for the comparisons between GA and Medium (pGA-Medium), GA and reference standard (pGA-reference standard), and reference standard and Medium (preference standard-Medium) greater than 0.05.

To carry out the analysis in a general manner, we computed the 6 p-values required to do pairwise comparisons of all 4 conditions for all probes using the ANOVA1 function in MATLAB. We then identified sets of probes matching the desired pattern. In the example above, probes were identified as being only affected by PG if their 6 pairwise comparison pvalues matched the following pattern (pGA-PG<0.05, pPG-reference standard<0.05, pPG-Medium<0.05, pGAMedium>0.05, pGA-reference standard>0.05, preference standard-Medium>0.05).

Cell Type Enrichment

To measure cell-type specificity in gene sets, we utilized Benita et al.'s enrichment tool (74) to calculate specificity scores relating each gene to each cell type in IMMGEN (Immunological Genome Project). (75) We then utilize a hyper-geometric test to calculate the significance of the summed specificity scores for the geneset across each cell type. Finally, we adjusted each pvalue output by the hypergeometric enrichment using the Benjamini-Hochberg False Discovery Rate correction for multiple hypothesis testing. (66)

Functional Enrichment with MSigDB

To assess the functional significance of lists of genes (test set), we used version 3.1 of the database of MSigDB (17) as our reference set. We implemented a standard hypergeometic enrichment test with the additional criterion that at least three genes from our test set be in the reference set. We then applied the Benjamini-Hochberg correction procedure and used a significance threshold of 0.05.

### Discussion

### EXAMPLES 5-11

We have developed a set of computational methods for comparing the immunological impact of an innovative medicine with that of a PG.

The first set of methods involves comparing the variability of samples in expression of certain genes. We applied a broad range of computational methods including a variance ratio analysis that identifies specific genes for which one medicine is very consistent and the other is highly variable. We compared variability for individual genes directly using an F-test, plotted the coefficient of variation as a function of intensity to determine the relationship between variability and probe intensity, and investigated variability across batches. We also developed a new method using principles from chemical/process engineering to determine variability using acceptable ranges defined by a reference standard.

These methods produced multiple lines of evidence suggesting that PG has a significantly more variable biological impact than either GA reference standard or GA. For instance, different GA batches were found to be highly consistent and similar to GA reference standard.

In contrast, more probes have higher variability in expression following stimulation with different PG batches. This variability itself is cause for concern among physicians and regulators, since the batch-to-batch variability of the product could manifest itself in ways that are harmful to patients. One possibility is that a patient could experience benefit from a particular batch of PG but not from a subsequent batch, preventing the patient from achieving the maximum benefit possible. Another, more disconcerting, possibility is that the variability could lead to a particular batch of PG causing adverse events. Due to PG's heterogeneity, such adverse events could be intermittent and therefore difficult to detect, monitor, and report.

The next set of methods involved identifying immunological impacts that differ between two medicines. We identified differentially expressed genes using a variety of methods (multiple parametric tests, non-parametric tests, and an ANOVA-based pattern matching method). We then explored the immunological relevance of these differentially expressed genes using a newly developed method for determining enrichment in genes specific to particular immune cell types. We further investigated the resulting hypotheses using established methods including hypergeometric enrichment with MSigDB (17) and GSEA (17) on lists of cell-type specific genes and transcription-factor target genes.

These methods identified specific genes and immune cell types that are upregulated significantly more by the GA than by PG. In this case, there is a preponderance of evidence suggesting that GA upregulates FoxP3+ Tregs more consistently and more effectively than PG. We have shown that the expression of FoxP3 itself, genes downstream of FoxP3, other known Treg markers, and Treg specific genes are all enriched from activation by GA relative to PG. This dramatic difference in biological impact on Tregs is certainly of note to physicians and regulators. It is well established that FoxP3+ Tregs induce beneficial tolerance in MS patients by suppressing harmful myelin reactive T cells, (54) so the more variable and reduced Treg induction raises questions about the potential efficacy of PG especially given recent findings demonstrating Copaxone's impact on Tregs (51) and linking Tregs to clinical response in MS patients. (55) These methods also identified specific genes and immune cell types that are upregulated significantly more by PG than by GA. In this case, PG had a significantly higher impact on cells of the myeloid lineage such as monocytes and macrophages than GA did. Genes with significantly higher expression in PG than in GA include key monocyte markers such as CD14, enrich to macrophage and monocyte cell types, and are enriched in related pathways such as TLR signaling. The stronger upregulation of monocyte-specific genes warrants further investigation by physicians and regulators, especially given that monocytes are “prominent contributors” to neuroinflammation in MS (56) and given recent reports that one of GA's mechanisms of action involves its impact on monocytes. (52)

The potential safety concerns stemming from PG's impact on monocytes were heightened by our GSEA analysis finding that the gene expression patterns following activation by PG more closely resemble the gene expression patterns of CD16dim monocytes, while the expression patterns following GA activation more closely resemble the gene expression patterns associated with CD16+ monocytes. This is consistent with literature reports showing that Copaxone positively impacts CD16+ monocytes, (57) and is particularly concerning from a safety perspective because the CD16dim monocytes favored by PG are known to play a different biological role.

The difference in impact on monocytes could also help explain the observed differences in Treg upregulation, since GA-treated monocytes are known to upregulate FoxP3 expression. (49) GA had an opposite impact on monocytes stimulated by LPS (resulting in increased IL1B production) as opposed to monocytes stimulated by T cell contact (resulting in decreased IL1B production). (52) The same PG samples that have unusually high levels of IL1B also have unusually low FoxP3. PG also shows upregulation in LPS response pathways. Together, these findings suggest that some component of PG, either deliberate or due to contamination, may trigger an LPS response pathway in monocytes leading to excessive IL1B production and unusually low induction of FoxP3+ Tregs. This possibility warrants further investigation with regard to safety.

One clear caveat to any gene expression study in mice lies in the inherent differences between healthy mouse models and human MS patients. Yet, there are clear differences in biological impact of GA and PG. One step to further address this lies in linking the differentially impact genes to markers and processes known to be linked to Copaxone response in humans with MS. (27, 28)

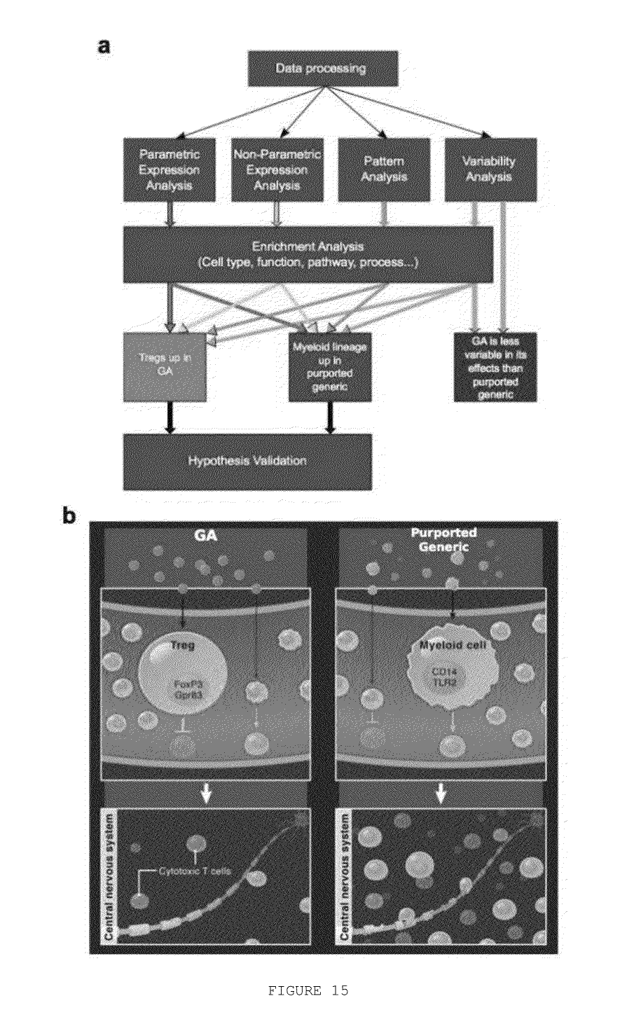

In these studies, we have sought to develop a broadly applicable set of computational methods for comparing innovative medicines to PGs (FIG. 15A). We found higher variability in gene expression following activation by PG compared to GA, and the significant differences in impact on key biological processes including Tregs and monocytes (FIG. 15B). These differences raise questions for physicians and regulators seeking safe and effective treatments for MS patients, and suggest that clinical studies are warranted, using appropriate safety and efficacy endpoints to comparing PG to GA. More generally, the data analysis methods described here can be utilized in a variety of situations to compare the immunological impact of two purportedly similar therapies, in order to ensure that patients receive the best possible medicines.

### REFERENCES

1. Noseworthy J H, Lucchinetti C, Rodriguez M, Weinshenker B G. Multiple sclerosis. N Engl J Med 2000; 343:938-52.

2. Guideline on clinical investigation of medicinal products for the treatment of multiple sclerosis EMEA, London 16 Sep. 2006.

3. Bjartmar C, Fox R J. Pathological mechanisms and disease progression of multiple sclerosis: therapeutic implications. Drugs of Today 2002; 38:17-29.

4. Fleming J O. Diagnosis and management of multiple sclerosis. 1st ed. New York: Professional communications, Inc., 2002.

5. Anderson D W, Ellenberg J H, Leventhal C M et al. Revised estimate of the prevalence of multiple sclerosis in the United States. Ann Neurol 1992; 31:333-36.

6. Compston A, Lassmann H, McDonald I. The story of multiple sclerosis. In: Compston A, Confavreux C, Lassman H, Mcdonald I, Miller D, Noseworthy J H, Smith K, Wekerle H, editors. McAlpine's Multiple Sclerosis. London: Churchill Livingstone; 2006. p. 3-68.

7. Revel M., Pharmacol. Ther., 100(1):49-62 (2003). 8. Copaxone® (glatiramer acetate injection) (2012) Teva Pharmaceutical Industries, Ltd Petach-Tikva, Israel:Rev-2/209.

9. Varkony H, et al. (2009) The glatiramoid class of immunomodulator drugs. Expert Opin Pharmacother 10:657-668.

10. Filippi M, et al. (2001) Glatiramer acetate reduces the proportion of new MS lesions evolving into “black holes”. Neurology 57:731-733.

11. Kipnis J, Schwartz M (2002) Dual action of glatiramer acetate (Cop-1) in the treatment of CNS autoimmune and neurodegenerative disorders. TRENDS in Molecular Medicine 8:319-323.

12. Teitelbaum D, Aharoni R, Arnon R, Sela M (1988) Specific inhibition of the T-cell response to myelin basic protein by the synthetic copolymer Cop 1. Proc Natl Acad Sci USA 85:9724-9728.

13. Putheti P, Soderstrom M, Link H, Huang Y M (2003) Effect of glatiramer acetate (Copaxone) on CD4+ CD25high T regulatory cells and their IL-10 production in multiple sclerosis. J Neuroimmunol 144:125-131.

14. Stadelmann C, et al. (2002) BDNF and gp145trkB in multiple sclerosis brain lesions: neuroprotective interactions between immune and neuronal cells? Brain 125:75-85.

15. Ford C, et al. (2010) Continuous long-term immunomodulatory therapy in relapsing multiple sclerosis: results from the 15-year analysis of the US prospective open-label study of glatiramer acetate. Mult Scler 16:342-350.

16. Aharoni R, Teitelbaum D, Arnon R, Sela M (1999) Copolymer 1 acts against the immunodominant epitope 82-100 of myelin basic protein by T cell receptor antagonism in addition to major histocompatibility complex blocking. Proc Natl Acad Sci USA 96:634-639.

17. Arnon R, Aharoni R (2004) Mechanism of action of glatiramer acetate in multiple sclerosis and its potential for the development of new applications. Proc Natl Acad Sci USA 101 Suppl 2:14593-14598.

18. Teitelbaum D, Aharoni R, Sela M, Arnon R (1991) Cross-reactions and specificities of monoclonal antibodies against myelin basic protein and against the synthetic copolymer 1. Proc Natl Acad Sci USA 88:9528-9532.

19. Webb C, Teitelbaum D, Arnon R, Sela M (1973) In vivo and in vitro immunological cross-reactions between basic encephalitogen and synthetic basic polypeptides capable of suppressing experimental allergic encephalomyelitis. Eur J Immunol 3:279-286.

20. Duda P W, et al. (2000) Human and murine CD4 T cell reactivity to a complex antigen: recognition of the synthetic random polypeptide glatiramer acetate. J Immunol 165:7300-7307.

21. Duda P W, et al. (2000) Glatiramer acetate (Copaxone) induces degenerate, Th2-polarized immune responses in patients with multiple sclerosis. J Clin Invest 105:967-976.

22. Brenner T, et al. (2001) Humoral and cellular immune responses to Copolymer 1 in multiple sclerosis patients treated with Copaxone. J Neuroimmunol 115:152-160.

23. Aharoni R, Teitelbaum D, Sela M, Arnon R (1997) Copolymer 1 induces T cells of the T helper type 2 that crossreact with myelin basic protein and suppress experimental autoimmune encephalomyelitis. Proc Natl Acad Sci USA 94:10821-10826.

24. Wiesemann E, et al. (2003) Correlation of serum IL-13 and IL-5 levels with clinical response to Glatiramer acetate in patients with multiple sclerosis. Clin Exp Immunol 133:454-460.

25. Aharoni R, Teitelbaum D, Sela M, Arnon R (1998) Bystander suppression of experimental autoimmune encephalomyelitis by T cell lines and clones of the Th2 type induced by copolymer 1. J Neuroimmunol 91:135-146.

26. Aharoni R, et al. (2003) Glatiramer acetate-specific T cells in the brain express T helper 2/3 cytokines and brain-derived neurotrophic factor in situ. Proc Natl Acad Sci USA 100:14157-14162.

27. Miller A, et al. (1998) Treatment of multiple sclerosis with copolymer-1 (Copaxone): implicating mechanisms of Th1 to Th2/Th3 immune-deviation. J Neuroimmunol 92:113-121.

28. Neuhaus O, et al. (2000) Multiple Sclerosis: Comparison of copolymer-1-reactive T cell Lines from treated and untreated subjects reveals cytokine shift From T helper 1 to T helper 2 cells. Proceedings of the National Academy of Sciences 97:7452-7457.

29. Venken K, et al. (2008) Natural naïve CD4+CD25+CD127low regulatory T cell (Treg) development and function are disturbed in multiple sclerosis patients: recovery of memory Treg homeostasis during disease progression. J Immunol 180:6411-6420.

30. Haas J, et al. (2009) Glatiramer acetate improves regulatory T-cell function by expansion of naive CD4(+)CD25(+)FOXP3(+)CD31(+) T-cells in patients with multiple sclerosis. J Neuroimmunol 216:113-117.

31. Hong J, et al. (2005) Induction of CD4+CD25+ regulatory T cells by copolymer-I through activation of transcription factor Foxp3. Proc Natl Acad Sci USA 102:6449-6454.

32. Weber M S, et al. (2007) Type II monocytes modulate T cell-mediated central nervous system autoimmune disease. Nat Med 13:935-943.

33. Xu D, et al. (1998) Selective expression and functions of interleukin 18 receptor on T helper (Th) type 1 but not Th2 cells. J Exp Med 188:1485-1492.

34. Jee Y, et al. (2007) CD4(+)CD25(+) regulatory T cells contribute to the therapeutic effects of glatiramer acetate in experimental autoimmune encephalomyelitis. Clin Immunol 125:34-42.

35. Windhagen A, et al. (1998) Cytokine secretion of myelin basic protein reactive T cells in patients with multiple sclerosis. J Neuroimmunol 91:1-9.

36. Neuhaus O, et al. (2000) Multiple sclerosis: comparison of copolymer-1-reactive T cell lines from treated and untreated subjects reveals cytokine shift from T helper 1 to T helper 2 cells. Proc Natl Acad Sci USA 97:7452-7457.

37. Chen M, et al. (2001) Glatiramer acetate induces a Th2-biased response and crossreactivity with myelin basic protein in patients with MS. Mult Scler 7:209-219.

38. Franciotta D, et al. (2003) Interferon gamma and interleukin 4 producing T cells in peripheral blood of multiple sclerosis patients undergoing immunomodulatory treatment. J Neurol Neurosurg Psychiatry 74:123-126.

39. Weder C, et al. (2005) Clinical and immune responses correlate in glatiramer acetate therapy of multiple sclerosis. Eur J Neurol 12:869-878.

40. Sanna A, et al. (2006) Glatiramer acetate reduces lymphocyte proliferation and enhances IL-5 and IL-13 production through modulation of monocyte-derived dendritic cells in multiple sclerosis. Clin Exp Immunol 143:357-362.

41. Kipnis J, Avidan H, Caspi R R, Schwartz M (2004) Dual effect of CD4+CD25+ regulatory T cells in neurodegeneration: a dialogue with microglia. Proc Natl Acad Sci USA 101 Suppl 2:14663-14669.

42. Tennakoon D K, et al. (2006) Therapeutic induction of regulatory, cytotoxic CD8+ T cells in multiple sclerosis. J Immunol 176:7119-7129.

43. Praksova P, et al. (2012) Immunoregulatory T cells in multiple sclerosis and the effect of interferon beta and glatiramer acetate treatment on T cell subpopulations. J Neurol Sci 319:18-23.

44. Achiron A, et al. (2009) Molecular profiling of glatiramer acetate early treatment effects in multiple sclerosis. Dis Markers. 27(2):63-73.

45. Dunning M J, Smith M L, Ritchie M E, Tavare S (2007) beadarray: R classes and methods for Illumina bead-based data. Bioinformatics 23:2183-2184.

46. Sharan R, Maron-Katz A, Shamir R (2003) CLICK and EXPANDER: a system for clustering and visualizing gene expression data. Bioinformatics 19:1787-1799.

47. Sugimoto N (2006) Foxp3-dependent and -independent molecules specific for CD25+CD4+ natural regulatory T cells revealed by DNA microarray analysis. International Immunology 18:1197-1209.

48. Subramanian N, et al. (2005) Gene set enrichment analysis: A knowledge-based approach for interpreting genome-wide expression profiles. Proc Natl Acad Sci USA 102:15545-15550.

49. Weber M. S. et al. (2007) Type II monocytes modulate T cell-mediated central nervous system autoimmune disease. Nature Medicine 13:935-943.

50, Wang Z. (2006) Role of IFN-g in induction of Foxp3 and conversion of CD4+CD25− f cells to CD4+ Tregs. Journal of Clinical Investigation. doi:10.1172/JCI25826.

51. Hong J. et al. (2005) Induction of CD4+CD25+ regulatory T cells by copolymer-I through activation of transcription factor Foxp3. Proc Natl Aced Sci USA 102:6449-6454.

52. Burger D. et al. (2009) Glatiramer acetate increases IL-1 receptor antagonist but decreases T cell-induced IL-lp in human monocytes and multiple sclerosis. Proceedings of the National Academy of Sciences 106:4355-4359.

53. Ingersoll M. A. et al. (2010) Comparison of gene expression profiles between human and mouse monocyte subsets. Blood 115, e10-e19.

54. Nylander A. and Hafler D. A. (2012) Multiple sclerosis. Journal of Clinical Investigation 122:1180-1188.

55. Dhib-Jalbut S. et al. (2012) in ECTRIMS.

56. Bar-Or A. et al. (2003) Analyses of all matrix metalloproteinase members in leukocytes emphasize monocytes as major inflammatory mediators in multiple sclerosis. Brain 126:2738-2749.

57. Pul R. et al. (2012) Glatiramer Acetate Increases Phagocytic

Activity of Human Monocytes In Vitro and in Multiple Sclerosis Patients. PLoS ONE 7, e51867.

58. Anis S. et al. (2013) in AAN.

59. Anis S. et al. (2010) in ECTRIMS.

60. Bolstad B. M., preprocessCore: A collection of pre-processing functions.

61. Johnson W. E. et al. (2007) Adjusting batch effects in microarray expression data using empirical Bayes methods. BiostatS 118-127.

62. Leek J. T. et al., sva: Surrogate Variable Analysis.

63. Ho J. W. K. et al. (2008) Differential variability analysis of gene expression and its application to human diseases. Bioinformatics 24:i390-398.

64. Quackenbush J. (2002) Microarray data normalization and transformation. Nature Genetics 32:496-501.

65. Pavlidis P. (2003) Using ANOVA for gene selection from microarray studies of the nervous system. Methods 31:282-289.

66. Benjamini Y. and Hochberg Y. (1995) Controlling the false discovery rate: a practical and powerful approach to multiple testing. Journal of the Royal Statistical Society. Series B (Methodological) 289-300.

67. Smyth G. K. (2004) Linear models and empirical bayes methods for assessing differential expression in microarray experiments. StatAppl Genet Mol Biol 3, Article3.

68. Smyth G. K. (2005) In Bioinformatics and Computational Biology Solutions Using R and Bioconductor, R. Gentleman, V. Carey, S. Dudoit, R. Irizarry, W. Huber, Eds. (Springer, New York) 397-420.

69. Gentleman R. C. et al. (2004) Bioconductor: Open software development for computational biology and bioinformatics. Genome Biology 5, R80.

70. Reich M. et al. (2006) GenePattern 2.0, Nature Genetics 38:500-501.

71. Saeys Y. et al. (2007) A review of feature selection techniques in bioinformatics. Bioinformatics 23:2507-2517.

72. Zheng Y. et al. (2007) Genome-wide analysis of Foxp3 target genes in developing and mature regulatory T cells. Nature 445:936-940.

73. Eppig J. T. et al. (2012) The Mouse Genome Database (MGD):

comprehensive resource for genetics and genomics of the laboratory mouse. Nucleic Acids Res 40:D881-D886.

74. Benita Y. et al. (2010) Gene enrichment profiles reveal T-cell development, differentiation, and lineage-specific transcription factors including ZBTB25 as a novel NF-AT repressor. Blood 115:5376-5384.

75. Painter M. W. et al. (2011) Transcriptomes of the B and T lineages compared by multi-platform microarray profiling. Immunol 186:3047-3057.

1. A process for characterizing a glatiramer acetate related drug substance or drug product comprising the steps of:
a) obtaining a batch of the glatiramer acetate related drug substance or drug product;
b) immunizing a mammal with a predetermined amount of a glatiramer acetate related drug substance or drug product;
c) preparing a culture of cells from the mammal of step b) at a predetermined time after immunization;
d) incubating cells from the culture of step c) with a predetermined amount of the glatiramer acetate drug related substance or drug product of step a); and
e) determining the level of expression of at least one gene selected from the group consisting of genes regulated by glatiramer acetate drug substance or drug product in Gene Expression Omnibus accession number GSE40566; determining the level of expression of at least one gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slcllal, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4;
determining the level of expression of at least one gene selected from the group consisting of CD40, CD86, GATA3, HLA-DMA, HLA-DMB, ICOS, IFNG, IFNGR2, IL2, IL13, IL4, IL18, IL12RB1, IL17A, IL17F, IL18R1, IL2RA, IL2RG, IL4R, IL6R, TBX21, TGFBR2, TNF, FOXP3, IL10RB, KLRD1, CD69, LTB, CD83, PRF1, CAMK2D, LTA, FSCN1, TLR7, CSF2, CCR7, FASLG, IL1A, CCL5, CD8B, CXCL10, TLR2, CCL4, TLR7, IGHA1, IL24, SOCS1, OAS1, JAK1, PTPN2, IFITM1, IF135, STAT2, BCL2, MVD, FDPS, SQLE, NSDHL, DHCR24, Acat2/Acat3, MSMO1, LSS, CYP51A1, NFKBIE, PIK3R1, PPP3CC, CD3D, IL2RB, PTEN, CD3G, ICOS, CAMK2D, NFAT5, LAT, ITK, H2-M2, FASLG, LIF, IGRA1, PRKACB, SGK1, MAPK11, TSC22D3, JUN, FKBP5, ADRB2, MAP3K1, MAPK12, POU2F1, SMARCA2, CDKN1A, TGFB3, HSP90AA1, DHCR24, CCR5, and CXCL9; determining the level of expression of at least one gene selected from the group consisting of Foxp3, Il2, Il1a, Il1b, C3, S100a8, S100a9, Cxcl2, Cxcl3, Ccl4, Ccl3 and Cd14; determining the level of expression of at least one gene selected from the group consisting of the genes presented in Table 8; determining the level of expression of at least one gene selected from the group consisting of the genes presented in Table 10; determining the level of expression of at least one gene selected from the group consisting of FoxP3, GPR83, CD14, TLR2, IFNG, CD40 and IL1B; determining the level of expression of at least one gene selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes,
or determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever,
thereby characterizing the glatiramer acetate related drug substance or drug product of step a).

2. (canceled)

3. A process for discriminating between glatiramer acetate related drug substances or drug products comprising the steps of:
i) characterizing two or more glatiramer acetate related drug substances or drug products according to the process of claim 1 to obtain characteristics of each of the glatiramer acetate related drug substances or drug products; and
ii) comparing the characteristics of the glatiramer acetate related drug substances or drug products obtained in step i),
thereby discriminating between the glatiramer acetate related drug substances or drug products.

4. The process of claim 1, wherein the mammal is a rodent.

5. The process of claim 1, wherein the culture of step c) is a primary culture.

6. The process of claim 1, wherein the glatiramer acetate related drug substance or drug product of step a) is glatiramer acetate drug substance or drug product, or a glatiramer acetate related drug substance or drug product other than glatiramer acetate drug substance or drug product.

7. (canceled)

8. The process of claim 1, wherein the glatiramer acetate related drug substance or drug product of step b) is glatiramer acetate drug substance or drug product, or a glatiramer acetate related drug substance or drug product other than glatiramer acetate drug substance or drug product.

9. (canceled)

10. The process of claim 1, wherein the glatiramer acetate related drug substance or drug product of step b) is the same glatiramer acetate related drug substance or drug product of step a), or wherein the glatiramer acetate related drug substance or drug product of step b) is a different glatiramer acetate related drug substance or drug product than the glatiramer acetate related drug substance or drug product of step a).

11. (canceled)

12. In a process for producing a drug product comprising a glatiramer acetate related drug substance or a process for releasing a drug product comprising a glatiramer acetate related drug substance, the improvement comprising the steps of:
i) characterizing the glatiramer acetate related drug substance or drug product according to the process of claim 1, wherein step e) comprises determining the level of expression of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and;
ii) discarding the batch of the glatiramer acetate related drug substance as unacceptable for inclusion in the drug product or discarding the batch of the glatiramer acetate related drug product as unacceptable for release if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l 3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard; discarding the batch of the glatiramer acetate related drug substance as unacceptable for inclusion in the drug product if the level of expression of a gene selected from the group consisting of the genes presented in Table 8 is not substantially identical to the level of expression of a reference standard; discarding the batch of the glatiramer acetate related drug substance as unacceptable for inclusion in the drug product if the level of expression of a gene selected from the group consisting of the genes presented in Table 10 is not substantially identical to the level of expression of a reference standard; discarding the batch of the glatiramer acetate related drug substance as unacceptable for inclusion in the drug product if the level of expression of a gene selected from the group consisting of GPR83, IFNG and Foxp3 is decreased or if the level of expression of a gene selected from the group consisting of CD14, CD40, TLR2 and IL1B is increased; discarding the batch of the glatiramer acetate related drug substance as unacceptable for inclusion in the drug product if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or discarding the batch of the glatiramer acetate related drug substance as unacceptable for inclusion in the drug product if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes.

13. In a process for producing a drug product comprising a glatiramer acetate related drug substance substance or a process for releasing a drug product comprising a glatiramer acetate related drug substance, the improvement comprising the steps of:
i) characterizing the glatiramer acetate related drug substance or drug product according to the process of claim 1, wherein step e) comprises determining the level of expression of at least one gene selected from the group consisting of Foxp3, Il2, Il1a, Il1b, C3, S100a8, S100a9, Cxcl2, Cxcl3, Ccl4, Ccl3 and Cd14;
ii) discarding the batch of the glatiramer acetate related drug substance as unacceptable for inclusion in the drug product or discarding the batch of the glatiramer acetate related drug product as unacceptable for release if the level of expression of FoxP3 is decreased relative to a reference standard or if the level of expression of at least one gene selected from the group consisting of Il2, Il1a, Il1b, C3, S100a8, S100a9, Cxcl2, Cxcl3, Ccl4, Ccl3 and Cd14 is increased relative to a reference standard.

14. In a process for producing a drug product comprising a glatiramer acetate related drug substance substance or a process for releasing a drug product comprising a glatiramer acetate related drug substance, the improvement comprising the steps of:
i) characterizing the glatiramer acetate related drug substance or drug product according to the process of claim 2, wherein step e) comprises determining the level of biological activity of the cells of step c) selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever;
ii) discarding the batch of the glatiramer acetate related drug substance as unacceptable for inclusion in the drug product or discarding the batch of the glatiramer acetate related drug product as unacceptable for release if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard.

15-17. (canceled)

18. A method of identifying suboptimal activity of a glatiramer acetate related drug substance or drug product comprising the steps of:
a) administering a glatiramer acetate related drug substance or drug product to a rodent;
b) determining the level of biological activity of the rodent selected from the group consisting of, immune response to antigen presenting cells, differentiation of effector lymphocytes, suppression of T lymphocytes, activation of Foxp3 positive regulatory T cells, expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever,
or determining the level of expression in the rodent of one or more genes selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpil, 4930583H14Rik, Ifit3, LOC667370, Klhdc1, Cd247, Igfbp4, Oas2, Bcl11b, Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slcllal, C3, Gpr84, Acta2, Lcn2, Hmox1, Tsab1, Ccl14, Il2, Inhba, Cxcl1, Serpinb2, Upp1, Gpr109a, Gp38, Il1b, Cxcl2, Il1a, Ccl3, 6720418B01Rik, 5830496L11Rik, Cd8b1, Fcgrt, LOC385615 and Scml4; determining the level of expression of one or more genes selected from the group consisting of the genes presented in Table 8; determining the level of expression of one or more genes selected from the group consisting of the genes presented in Table 10; determining the level of expression of one or more genes selected from the group consisting of FoxP3, GPR83, CD14, TLR2, IFNG, CD40 and IL1B; determining the level of expression of one or more genes selected from the group consisting of the genes presented in Table 12; or determining gene set enrichment analysis for genes associated with at least one cell type selected from the group consisting of FoxP3+ CD4+ T cells, CD4+ T cells CD8+ T cells, gamma delta T cells, natural killer T cells, CD4+ CD8+ T cells, macrophage cells, monocyte cells stromal cells, multi-lineage progenitor cells, dendritic cells, fibroblastic reticular cells, fibroblasts and granulocytes; and
c) identifying the glatiramer acetate related drug substance or drug product as causing a suboptimal activity if the level of biological activity selected from the group consisting of immune response to antigen presenting cells, differentiation of effector lymphocytes, suppression of T lymphocytes and activation of Foxp3 positive regulatory T cells is decreased relative to a reference standard or if the level of biological activity selected from the group consisting of expansion of mononuclear leukocytes, proliferation of T lymphocytes, expansion of lymphocytes, differentiation of naïve lymphocytes, inflammatory response, adhesion of immune cells, cell movement, migration of cells, chemotaxis of cells, cell movement of phagocytes, chemotaxis of monocytes, cell movement of monocytes and fever is increased relative to a reference standard,
or identifying the glatiramer acetate related drug substance or drug product as causing a suboptimal activity if the level of expression of a gene selected from the group consisting of Ecm1, Pres1, Pdlim4, Gpr83, Ifng, Il24, LOC100046608, Gm590, Gpr114, Tmie, Rasgrp1, Myo6, Pfkp, Usp18, Arl4c, Als2cl, 2810410P22Rik, Arl5a, Gbp2, Rasgrp1, Ankrd37, Tpi1, 4930583H14Rik, Ifit3, LOC667370, Klhdc1, Cd247, Igfbp4, Oas2 Bcl11b, 6720418B01Rik, 5830496L11Rik, Cd8b1, Fcgrt, LOC385615 and Scml4 is decreased relative to a reference standard or if the level of expression of a gene selected from the group consisting of Fscn1, Ctsg, Mpo, Prtn3, Lyzs, Emr1, Chi3l1, Anxa3, Hp, Lyz2, Lyz, Fer1l3, Sirpa, Cd63, Clec4n, Clec4d, EG433016, Stfa1, Chi3l3 Ngp, S100a8, S100a9, Clecsf9, Saa3, 5033414K04Rik, Slc7a11, Slpi, Cd14, Fpr2, Fcgr3, F10, Gpnmb, Tgfbi, Mmp14, Slc11a1, C3, Gpr84, Acta2, Lcn2, Hmox1, Tsab1, Ccl4, Il2, Inhba, Cxcl1, Serpinb2, Upp1, Gpr109a, Gp38, Il1b, Cxcl2, Il1a, and Ccl3, is increased relative to a reference standard;
identifying the glatiramer acetate related drug substance or drug product as causing a suboptimal activity if the level of expression of a gene selected from the group consisting of the genes presented in Table 8 is not substantially identical to the level of expression of a reference standard; identifying the glatiramer acetate related drug substance or drug product as causing a suboptimal activity if the level of expression of a gene selected from the group consisting of the genes presented in Table 10 is not substantially identical to the level of expression of a reference standard; identifying the glatiramer acetate related drug substance or drug product as causing a suboptimal activity if the level of expression of a gene selected from the group consisting of GPR83, IFNG and Foxp3 is decreased or if the level of expression of a gene selected from the group consisting of CD14, CD40, TLR2 and IL1B is increased; identifying the glatiramer acetate related drug substance or drug product as causing a suboptimal activity if the level of expression of a gene selected from the group consisting of the genes identified in Table 12 as FoxP3+ T cell genes is decreased or if the level of expression of a gene selected from the group consisting of the genes identified in Table 12 as macrophage genes and the genes identified in Table 12 as monocyte genes is increased; or identifying the glatiramer acetate related drug substance or drug product as causing a suboptimal activity if gene set enrichment analysis indicates downregulation or a lack of upregulation for genes associated with at least one cell type selected from the group consisting of FoxP3+ CD4+ T cells, CD4+ T cells CD8+ T cells, gamma delta T cells, natural killer T cells and CD4+ CD8+ T cells or if gene set enrichment analysis indicates upregulation or a lack of downregulation for genes associated with at least one cell type selected from the group consisting of macrophage cells, monocyte cells stromal cells, multi-lineage progenitor cells, dendritic cells, fibroblastic reticular cells, fibroblasts and granulocytes
thereby identifying suboptimal activity of a glatiramer acetate related drug substance or drug product.

19. (canceled)

20. The method of claim 18, wherein the level of expression is determined in the blood.

21. The method of claim 20, wherein the level of expression is determined in PBMCs.

22. The method of claim 18, wherein the reference standard is the level of expression prior to administration of the glatiramer acetate related drug substance or drug product or the level of expression after administration of glatiramer acetate drug substance or drug product.

23. (canceled)

24. The process of claim 4, wherein the rodent is a mouse, wherein the rodent is a mouse and the mouse is a female (SJL×BALB/C) F1 mouse and/or wherein the rodent is a mouse and the mouse is about 8 to about 12 weeks old.

25-26. (canceled)

27. The process of claim 1, wherein the primary culture is a culture of spleen cells or a culture of lymph node cells.

28. (canceled)

29. The process of claim 27, wherein the primary culture is a culture of spleen cells and wherein the culture of spleen cells is prepared about 3 days after immunization.

30. The process of claim 1, wherein the incubation of step d) is for about 24 hours.

31. The process or method of any one of claims 1-30, wherein the glatiramer acetate related drug substance is a glatiramoid or wherein the glatiramer acetate related drug product comprises a glatiramoid, or wherein the glatiramer acetate related drug substance is a glatiramoid other than glatiramer acetate drug substance or wherein the glatiramer acetate related drug product comprises a glatiramoid other than glatiramer acetate drug substance.

32-42. (canceled)

43. The process of claim 12, wherein the reference standard is medium.
